(19) United States
(12) Patent Application Publication
(10) Pub. No.: US 2026/0077310 A1
(43) Pub. Date: Mar. 19, 2026

(54) BACTERIAL COMPOSITIONS FOR CONSUMING METHANE AND FIXING NITROGEN

(71) Applicant: Windfall Bio, Inc., San Mateo, CA (US)

(72) Inventors: Joshua A. SILVERMAN, Los Altos Hills, CA (US); Judy S. SU, Newark, CA (US); Carla RISSO, San Carlos, CA (US); Jordan W. Genz SMITH, Sacramento, CA (US)

(21) Appl. No.: 19/332,805

(22) Filed: Sep. 18, 2025

Related U.S. Application Data
(63) Continuation of application No. PCT/US2024/020785, filed on Mar. 20, 2024.
(60) Provisional application No. 63/453,739, filed on Mar. 21, 2023.
(60) Provisional application No. 63/578,591, filed on Aug. 24, 2023.
(60) Provisional application No. 63/605,960, filed on Dec. 4, 2023.
(60) Provisional application No. 63/563,784, filed on Mar. 11, 2024.

(51) Int. Cl. B01D 53/85; B01D 53/54; B01D 53/72; B01D 53/78; B01D 53/81; C12N 1/04; C12N 1/20; C12R 1/01

(52) U.S. Cl. CPC B01D 53/85; B01D 53/54; B01D 53/72; B01D 53/78; B01D 53/81; C12N 1/04; C12N 1/20; B01D 2257/102; B01D 2257/7025; B01D 2258/02; B01D 2258/05; C12R 2001/01

(57) ABSTRACT

The disclosure provided herein describes a composition comprising a consortium of bacteria that is capable of consuming methane (CH₄) and/or fixing nitrogen (N₂) at a high rate.

### CROSS-REFERENCE

This application is a continuation of International Patent Application No. PCT/US2024/020785, filed Mar. 20, 2024, which claims the benefit of U.S. Provisional Patent Application No. 63/453,739 filed Mar. 21, 2023, U.S. Provisional Application No. 63/578,591 filed Aug. 24, 2023, U.S. Provisional Application No. 63/605,960 filed Dec. 4, 2023, and U.S. Provisional Application No. 63/563,784 filed Mar. 11, 2024, each of which is incorporated by reference herein in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

The climate crisis is a great challenge. Over the past two decades, the world has mobilized around reducing and sequestering CO₂. Methane (CH₄) is a potent and increasingly prevalent greenhouse gas that warms the planet faster and sooner than CO₂. By 2050, methane is expected to be responsible for 0.5 degrees of warming. Decreasing methane in the atmosphere is expected to be one of the fastest and biggest mitigation strategies to combat the climate crisis. Further, climate change and farming practices have resulted in the loss of topsoil and soil-web diversity. Consequently, soil health has critically degraded and the soil itself is eroding 10-1000 times faster than it forms.

Given these challenges, there is a need to remove methane from the atmosphere and to convert it into a resource that can nourish soil and the plants grown in the soil. Disclosed herein are bacterial consortiums that are capable of consuming methane from natural and industrial sources while also fixing nitrogen to provide nutrients to soils and plants.

### SUMMARY

Disclosed herein is a composition comprising a consortium of bacteria. In some embodiments, the consortium of bacteria is capable of consuming methane (CH₄) at a rate greater than 13.5 umol/gDW per day and fixing nitrogen (N₂) at a rate greater than 2.0 umol/gDW per day. In some embodiments, the consortium of bacteria is capable of consuming methane (CH₄) at a rate greater than 5 umol/gDW per day and fixing nitrogen (N₂) at a rate greater than 2.5 umol/gDW per day.

In some embodiments, a first bacterium in the consortium of bacteria is unaffected by a second bacterium in the consortium of bacteria in respect to the first bacterium's ability to fix nitrogen. In some embodiments, a first bacterium in the consortium of bacteria is unaffected by a second bacterium in the consortium of bacteria in respect to the first bacterium's ability to consume CH₄.

In some embodiments, the consortium of bacteria comprises at least 1, 2, 3, 4, or more different bacterial phyla. In some embodiments, the consortium of bacteria comprises Micrarchaeota, Actinobacteria, Bacteroidetes, Firmicutes, Proteobacteria, or a combination thereof. In some embodiments, the consortium of bacteria comprises at least 1, 2, 3, 4, 5, 6, 7, 8, 9, 10, 15, 20, 30, or more different bacterial order. In some embodiments, the consortium of bacteria comprises Rhizobiales, Methylococcales, Methanobacteriales, Methanosarcinales, Nitrosomonadales, Pseudomonadale, or a combination thereof. In some embodiments, the consortium of bacteria comprises bacteria from at least 1, 2, 3, 4, 5, 6, 7, 8, 9, 10, 15, 20, 30, 40, 50, 60 or more different bacterial family. In some embodiments, the consortium of bacteria comprises Methylocystaceae, Methylococcaceae, Methanobacteriaceae, Methanosarcinaceae, Methylophilaceae or Pseudonocardiaceae, or a combination thereof. In some embodiments, the consortium of bacteria comprises bacteria from at least 1, 2, 3, 4, 5, 6, 7, 8, 9, 10, 15, 20, 30, 40, 50 or more different bacterial genera. In some embodiments, the consortium of bacteria comprises bacteria from at least 1, 2, 3, 4, 5, 6, 7, 8, 9, 10, 15, 20, 30, 40, 50 or more different species.

In some embodiments, the consortium of bacteria comprises species that are methanotrophs, nitrogen fixing bacteria, or both. In some embodiments, the consortium of bacteria comprises species from Tables 1, 3, or 4. In some embodiments, the consortium of bacteria comprises at least one or more species of bacteria listed in Table 5. In some embodiments, the consortium of bacteria comprises the species listed in Table 5. In some embodiments, the consortium of bacteria comprises at least one bacterial species that consumes CH₄ and fixes N₂. In some embodiments, the consortium of bacteria comprises Sphingopyxis terrae subsp terrae YC-JH3. In some embodiments, the consortium of bacteria comprises Methylomonas methanica R-45371. In some embodiments, the consortium of bacteria comprises Pseudoxanthomonas mexicana GTZY. In some embodiments, the consortium of bacteria comprises Sphingomonadaceae bacterium. In some embodiments, the consortium of bacteria comprises Shinella sp. HZN7. In some embodiments, the consortium of bacteria comprises Methylococcus capsulatus Bath. In some embodiments, the consortium of bacteria comprises Sphingopyxis sp. A083. In some embodiments, the consortium of bacteria comprises Methylobacter marinus A45. In some embodiments, the consortium of bacteria comprises Methylobacter whittenburyi UCM-B-3033. In some embodiments, the consortium of bacteria comprises Methylobacter sp. BBA5.1. In some embodiments, the consortium of bacteria comprises Methylosinus trichosporium OB3b. In some embodiments, the consortium of bacteria comprises Hyphomicrobium zavarzinii ATCC 27496 ZV-622. In some embodiments, the consortium of bacteria comprises Methylomonas methanica R-45371. In some embodiments, the consortium of bacteria comprises Pseudoxanthomonas. In some embodiments, the consortium of bacteria comprises Flavobacterium. In some embodiments, the consortium of bacteria comprises Sphingopyxis. In some embodiments, the consortium of bacteria comprises Methylomonas. In some embodiments, the consortium of bacteria comprises Chryseobacterium.

In some embodiments, the consortium of bacteria comprises a distribution of bacterial species, strains, families, phyla, or combination thereof and wherein the distribution changes over time. In some embodiments, the consortium of bacteria comprises a distribution of bacterial species, strains, families, phyla, or combination thereof and wherein the distribution does not change over time.

In some embodiments, the consortium of bacteria further comprises one or more additives. In some embodiments, the one or more additives improves viability of the consortium of bacteria. In some embodiments, the one or more additives are in a sufficient amount to keep at least one bacterial species of the consortium of bacteria capable of consuming CH₄ at a population frequency of at least 0.01%, 0.1%, or 1% for at least about 4 weeks. In some embodiments, the one or more additives increase CH₄ consumption or N₂ fixation as compared to a reference composition comprising the consortium of bacteria and lacking the one or more additives. In some embodiments, the one or more additives are present in an amount sufficient to favor consumption of the CH₄ by the consortium of bacteria over consumption of another organic molecule. In some embodiments, the one or more additive is present in the consortium of bacteria at a concentration of at least about 1 ug/L, about 10 ug/L, about 100 ug/L, or about 1 mg/L. In some embodiments, the one or more additives comprises at least one non-metal additive. In some embodiments, the at least one non-metal additive comprises phosphorus, ethylenediaminetetraacetic acid, or a combination thereof. In some embodiments, the one or more additives comprises at least one metal additive. In some embodiments, the one or more additives comprises at least one metallic salt additive. In some embodiments, the at least one metal additive comprises copper, tungsten, iron, nickel, molybdenum, cerium, manganese, lanthanum, zinc, cobalt, boron, or a combination thereof. In some embodiments, the one or more additives comprises copper.

In some embodiments, the consortium of bacteria comprises a bacterial species that expresses methane monooxygenase. In some embodiments, the consortium of bacteria comprises a bacterial species that expresses methyl coenzyme M reductase. In some embodiments, CH₄ consumption is determined by measuring a concentration of CH₄. In some embodiments, the concentration of CH₄ is determined using labeled CH₄. In some embodiments, the concentration of CH₄ is determined using radiolabeled CH₄. In some embodiments, the radiolabeled CH₄ comprises ³H-CH₄. In some embodiments, the radiolabeled CH₄ comprises ¹⁴C-CH₄. In some embodiments, the radiolabeled CH₄ comprises ¹³C-CH₄. In some embodiments, CH₄ consumption is determined by measuring a concentration of CO₂. In some embodiments, CH₄ consumption is determined by measuring a concentration of H₂O. In some embodiments, CH₄ consumption is determined by gas chromatography. In some embodiments, the gas chromatography comprises flame ionization detection gas chromatography. In some embodiments, CH₄ consumption is determined by a methane detector. In some embodiments, CH₄ consumption is determined by a portable methane detector.

In some embodiments, the consortium of bacteria increases N₂ fixation in low oxygen conditions. In some embodiments, the consortium of bacteria comprises at least one bacterial species that expresses nitrogenase. In some embodiments, N₂ fixation is determined by measuring a concentration of N₂. In some embodiments, the concentration of N₂ is determined using labeled N₂. In some embodiments, the concentration of N₂ is determined using radiolabeled N₂. In some embodiments, the radiolabeled N₂ comprises 13N-N₂. In some embodiments, the radiolabeled N₂ comprises ¹⁵N-N₂. In some embodiments, N₂ fixation is determined by measuring a concentration of NH₃. In some embodiments, N₂ fixation is determined by measuring a concentration of NH₄. In some embodiments, N₂ fixation is determined by measuring a concentration of CH₄. In some embodiments, N₂ fixation is determined by measuring a concentration of C₂H₄. In some embodiments, N₂ fixation is determined by gas chromatography. In some embodiments, the gas chromatography comprises flame ionization detection gas chromatography. In some embodiments, N₂ fixation is determined by an acetylene reduction assay. In some embodiments, N₂ fixation is determined by measuring a concentration of total nitrogen. In some embodiments, N₂ fixation is determined by combustion. In some embodiments, N₂ fixation is determined using a Dumas combustion method.

In some embodiments, the consortium of bacteria has viability for at least about 4 months or at least about 6 months. In some embodiments, the consortium of bacteria has viability for at least about 12 months. In some embodiments, the consortium of bacteria is stored at or about room temperature. In some embodiments, the consortium of is stored at or about 4° C. In some embodiments, the consortium of bacteria is stored at or about −20° C. In some embodiments, the consortium of bacteria comprises a neutral pH. In some embodiments, the consortium of bacteria comprises a basic pH. In some embodiments, the consortium of bacteria comprises an acidic pH. In some embodiments, the consortium of bacteria is freeze dried. In some embodiments, the consortium of bacteria is spray dried. In some embodiments, the consortium of bacteria is vacuum sealed.

In some embodiments, the consortium of bacteria disclosed herein comprises a kit. In some embodiments, a kit comprises the consortium of bacteria disclosed herein and instructions for use.

In some embodiments, the consortium of bacteria disclosed herein comprises a compost. In some embodiments, the compost comprises the consortium of bacteria. In some embodiments, the compost further comprises biomass. In some embodiments, the compost is enhanced with nitrogen compared to a reference compost that is substantially identical to the compost except the reference compost does not comprise the consortium of bacteria. In some embodiments, the compost improves nitrogen availability of soil when applied to the soil.

In some embodiments, the consortium of bacteria disclosed herein comprises a method for treating a crop. In some embodiments, the method for treating a crop comprises applying to the crop, to soil in which the crop is grown, or to liquid medium used to grow the crop the composition of the consortium of bacteria disclosed herein. In some embodiments, the crop is an agricultural crop. In some embodiments, the crop comprises one or more plants.

In some embodiments, the consortium of bacteria disclosed herein comprises use in a bioreactor. In some embodiments, a bioreactor comprises the composition of bacteria as disclosed herein.

In some embodiments, the consortium of bacteria disclosed herein comprises use in a system. In some embodiments, the system comprises a reaction chamber, a first fluid input in fluid communication with the reaction chamber, wherein the first fluid input is configured to direct gas enriched in methane (CH₄) into the reaction chamber, a second fluid input in fluid communication with the reaction chamber, wherein the second fluid input is configured to direct the consortium of bacteria as disclosed herein into the reaction chamber, and an outlet in fluid communication with the reaction chamber, wherein the enriched CH₄ is at a concentration of at least 1.89 parts per million (ppm). In some embodiments, the enriched CH₄ is from a storage of concentrated CH₄. In some embodiments, the enriched CH₄ is from an animal housing or free-range ruminants. In some embodiments, the enriched CH₄ is from any industrial source. In some embodiments, the industrial source comprises a natural gas pipeline, a syngas reactor, a reactor, or a refinery. In some embodiments, the enriched CH₄ is from any natural source. In some embodiments, the first fluid input and the second fluid input are the same input. In some embodiments, the first fluid input and the second fluid input are different from one another.

Disclosed herein is a composition comprising a consortium of bacteria. In some embodiments, the consortium of bacteria is capable of fixing nitrogen (N₂). In some embodiments, the consortium of bacteria is capable of consuming methane (CH₄). In some embodiments, the consortium of bacteria is capable of fixing nitrogen (N₂) and consuming methane (CH₄). In some embodiments, the consortium of bacteria is capable of fixing nitrogen (N₂) and consuming methane (CH₄), wherein the consortium of bacteria has viability for at least about 3 months.

In some embodiments, the consortium of bacteria consumes methane (CH₄) at a rate greater than 5 umol/gDW per day. In some embodiments, the consortium of bacteria consumes methane (CH₄) at a rate greater than 13.5 umol/gDW per day. In some embodiments, the consortium of bacteria fixes nitrogen (N₂) at a rate greater than 2.0 umol/gDW per day. In some embodiments, the consortium of bacteria fixes nitrogen (N₂) at a rate greater than 2.5 umol/gDW per day.

In some embodiments, a first bacterium in the consortium of bacteria is unaffected by a second bacterium in the consortium of bacteria in respect to the first bacterium's ability to fix nitrogen. In some embodiments, a first bacterium in the consortium of bacteria is unaffected by a second bacterium in the consortium of bacteria in respect to the first bacterium's ability to consume CH₄.

In some embodiments, the consortium of bacteria comprises at least 1, 2, 3, 4, or more different bacterial phyla. In some embodiments, the consortium of bacteria comprises Micrarchaeota, Actinobacteria, Bacteroidetes, Firmicutes, Proteobacteria, or a combination thereof. In some embodiments, the consortium of bacteria comprises at least 1, 2, 3, 4, 5, 6, 7, 8, 9, 10, 15, 20, 30, or more different bacterial order. In some embodiments, the consortium of bacteria comprises Rhizobiales, Methylococcales, Methanobacteriales, Methanosarcinales, Nitrosomonadales, Pseudomonadale, or a combination thereof. In some embodiments, the consortium of bacteria comprises bacteria from at least 1, 2, 3, 4, 5, 6, 7, 8, 9, 10, 15, 20, 30, 40, 50, 60 or more different bacterial family. In some embodiments, the consortium of bacteria comprises Methylocystaceae, Methylococcaceae, Methanobacteriaceae, Methanosarcinaceae, Methylophilaceae, Pseudonocardiaceae, or a combination thereof. In some embodiments, the consortium of bacteria comprises bacteria from at least 1, 2, 3, 4, 5, 6, 7, 8, 9, 10, 15, 20, 30, 40, 50 or more different bacterial genera. In some embodiments, the consortium of bacteria comprises bacteria from at least 1, 2, 3, 4, 5, 6, 7, 8, 9, 10, 15, 20, 30, 40, 50 or more different species.

In some embodiments, the consortium of bacteria comprises species that are methanotrophs, nitrogen fixing bacteria, or both. In some embodiments, the consortium of bacteria comprises species from Tables 1, 3, or 4. In some embodiments, the consortium of bacteria comprises at least one or more species of bacteria listed in Table 5. In some embodiments, the consortium of bacteria comprises the species listed in Table 5. In some embodiments, the consortium of bacteria comprises at least one bacterial species that consumes CH₄ and fixes N₂.

In some embodiments, the consortium of bacteria further comprises one or more additives. In some embodiments, the one or more additives improve viability of the consortium of bacteria. In some embodiments, the one or more additives are in a sufficient amount to keep at least one bacterial species of the consortium of bacteria capable of consuming CH₄ at a population frequency of at least 0.01%, 0.1%, or 1% for at least about 4 weeks. In some embodiments, the one or more additives increase CH₄ consumption or N₂ fixation as compared to a reference composition comprising the consortium of bacteria and lacking the one or more additives. In some embodiments, the one or more additives are present in an amount sufficient to favor consumption of the CH₄ by the consortium of bacteria over consumption of another organic molecule. In some embodiments, the one or more additives are present in the consortium of bacteria at a concentration of at least about 1 ug/L, about 10 ug/L, about 100 ug/L, or about 1 mg/L. In some embodiments, the one or more additives comprises at least one non-metal additive. In some embodiments, the at least one non-metal additive comprises phosphorus, ethylenediaminetetraacetic acid, or a combination thereof. In some embodiments, the one or more additives comprises at least one metal additive. In some embodiments, the one or more additives comprises at least one metallic salt additive. In some embodiments, the at least one metal additive comprises copper, tungsten, iron, nickel, molybdenum, cerium, manganese, lanthanum, zinc, cobalt, boron, or a combination thereof. In some embodiments, the one or more additives comprises copper.

In some embodiments, the consortium of bacteria comprises a bacterial species that expresses methane monooxygenase. In some embodiments, the consortium of bacteria comprises a bacterial species that expresses methyl coenzyme M reductase. In some embodiments, CH₄ consumption is determined by measuring a concentration of CH₄. In some embodiments, the concentration of CH₄ is determined using labeled CH₄. In some embodiments, the concentration of CH₄ is determined using radiolabeled CH₄. In some embodiments, the radiolabeled CH₄ comprises ³H-CH₄. In some embodiments, the radiolabeled CH₄ comprises ¹⁴C-CH₄. In some embodiments, the radiolabeled CH₄ comprises ¹³C-CH₄. In some embodiments, CH₄ consumption is determined by measuring a concentration of CO₂. In some embodiments, CH₄ consumption is determined by measuring a concentration of H₂O. In some embodiments, CH₄ consumption is determined by gas chromatography. In some embodiments, the gas chromatography comprises flame ionization detection gas chromatography. In some embodiments, CH₄ consumption is determined by a methane detector. In some embodiments, CH₄ consumption is determined by a portable methane detector.

In some embodiments, the consortium of bacteria increases N₂ fixation in low oxygen conditions. In some embodiments, the consortium of bacteria comprises at least one bacterial species that expresses nitrogenase. In some embodiments, N₂ fixation is determined by measuring a concentration of N₂. In some embodiments, the concentration of N₂ is determined using labeled N₂. In some embodiments, the concentration of N₂ is determined using radiolabeled N₂. In some embodiments, the radiolabeled N₂ comprises 13N-N₂. In some embodiments, the radiolabeled N₂ comprises ¹⁵N-N₂. In some embodiments, N₂ fixation is determined by measuring a concentration of NH₃. In some embodiments, N₂ fixation is determined by measuring a concentration of NH₄. In some embodiments, N₂ fixation is determined by measuring a concentration of CH₄. In some embodiments, N₂ fixation is determined by measuring a concentration of C₂H₄. In some embodiments, N₂ fixation is determined by gas chromatography. In some embodiments, the gas chromatography comprises flame ionization detection gas chromatography. In some embodiments, N₂ fixation is determined by an acetylene reduction assay. In some embodiments, N₂ fixation is determined by measuring a concentration of total nitrogen. In some embodiments, N₂ fixation is determined by combustion. In some embodiments, N₂ fixation is determined using a Dumas combustion method.

In some embodiments, the consortium of bacteria has viability for at least about 4 months or at least about 6 months. In some embodiments, the consortium of bacteria has viability for at least about 12 months. In some embodiments, the consortium of bacteria is stored at or about room temperature. In some embodiments, the consortium of is stored at or about 4° C. In some embodiments, the consortium of bacteria is stored at or about −20° C. In some embodiments, the consortium of bacteria comprises a neutral pH. In some embodiments, the consortium of bacteria comprises a basic pH. In some embodiments, the consortium of bacteria comprises an acidic pH. In some embodiments, the consortium of bacteria is freeze dried. In some embodiments, the consortium of bacteria is spray dried. In some embodiments, the consortium of bacteria is vacuum sealed.

In some embodiments, the consortium of bacteria disclosed herein comprises a kit. In some embodiments, a kit comprises the consortium of bacteria disclosed herein and instructions for use.

In some embodiments, the consortium of bacteria disclosed herein comprises a compost. In some embodiments, the compost comprises the consortium of bacteria. In some embodiments, the compost further comprises biomass. In some embodiments, the compost is enhanced with nitrogen compared to a reference compost that is substantially identical to the compost except the reference compost does not comprise the consortium of bacteria. In some embodiments, the compost improves nitrogen availability of soil when applied to the soil.

In some embodiments, the consortium of bacteria disclosed herein comprises a method for treating a crop. In some embodiments, the method for treating a crop comprises applying to the crop, to soil in which the crop is grown, or to liquid medium used to grow the crop the composition of the consortium of bacteria disclosed herein. In some embodiments, the crop is an agricultural crop. In some embodiments, the crop comprises one or more plants.

In some embodiments, the consortium of bacteria disclosed herein comprises use in a bioreactor. In some embodiments, a bioreactor comprises the composition of bacteria as disclosed herein.

In some embodiments, the consortium of bacteria disclosed herein comprises use in a system. In some embodiments, a system comprises a reaction chamber, a first fluid input in fluid communication with the reaction chamber, wherein the first fluid input is configured to direct gas enriched in methane (CH₄) into the reaction chamber, a second fluid input in fluid communication with the reaction chamber, wherein the second fluid input is configured to direct the consortium of bacteria as disclosed herein into the reaction chamber, and an outlet in fluid communication with the reaction chamber, wherein the enriched CH₄ is at a concentration of at least 1.89 parts per million (ppm). In some embodiments, the enriched CH₄ is from a storage of concentrated CH₄. In some embodiments, the enriched CH₄ is from an animal housing or free-range ruminants. In some embodiments, the enriched CH₄ is from any industrial source. In some embodiments, the industrial source comprises a natural gas pipeline, a syngas reactor, a reactor, or a refinery. In some embodiments, the enriched CH₄ is from any natural source. In some embodiments, the first fluid input and the second fluid input are the same input. In some embodiments, the first fluid input and the second fluid input are different from one another.

Disclosed herein is a composition comprising a consortium of bacteria. In some embodiments, the consortium of bacteria is capable of fixing nitrogen (N₂) and consuming methane (CH₄). In some embodiments, the consortium of bacteria is capable of fixing nitrogen (N₂) and consuming methane (CH₄), wherein the consortium has at least one bacterial species from Table 4.

In some embodiments, the consortium of bacteria consumes methane (CH₄) at a rate greater than 5 umol/gDW per day. In some embodiments, the consortium of bacteria consumes methane (CH₄) at a rate greater than 13.5 umol/gDW per day. In some embodiments, the consortium of bacteria fixes nitrogen (N₂) at a rate greater than 2.0 umol/gDW per day. In some embodiments, the consortium of bacteria fixes nitrogen (N₂) at a rate greater than 2.5 umol/gDW per day.

In some embodiments, a first bacterium in the consortium of bacteria is unaffected by a second bacterium in the consortium of bacteria in respect to the first bacterium's ability to fix nitrogen. In some embodiments, a first bacterium in the consortium of bacteria is unaffected by a second bacterium in the consortium of bacteria in respect to the first bacterium's ability to consume CH₄.

In some embodiments, the consortium of bacteria comprises at least 1, 2, 3, 4, or more different bacterial phyla. In some embodiments, the consortium of bacteria comprises Micrarchaeota, Actinobacteria, Bacteroidetes, Firmicutes, Proteobacteria, or a combination thereof. In some embodiments, the consortium of bacteria comprises at least 1, 2, 3, 4, 5, 6, 7, 8, 9, 10, 15, 20, 30, or more different bacterial order. In some embodiments, the consortium of bacteria comprises Rhizobiales, Methylococcales, Methanobacteriales, Methanosarcinales, Nitrosomonadales, Pseudomonadale, or a combination thereof. In some embodiments, the consortium of bacteria comprises bacteria from at least 1, 2, 3, 4, 5, 6, 7, 8, 9, 10, 15, 20, 30, 40, 50, 60 or more different bacterial family. In some embodiments, the consortium of bacteria comprises Methylocystaceae, Methylococcaceae, Methanobacteriaceae, Methanosarcinaceae, Methylophilaceae, Pseudonocardiaceae, or a combination thereof. In some embodiments, the consortium of bacteria comprises bacteria from at least 1, 2, 3, 4, 5, 6, 7, 8, 9, 10, 15, 20, 30, 40, 50 or more different bacterial genera. In some embodiments, the consortium of bacteria comprises bacteria from at least 1, 2, 3, 4, 5, 6, 7, 8, 9, 10, 15, 20, 30, 40, 50 or more different species.

In some embodiments, the consortium of bacteria comprises species that are methanotrophs, nitrogen fixing bacteria, or both. In some embodiments, the consortium of bacteria comprises species from Tables 1, 3, or 4. In some embodiments, the consortium of bacteria comprises at least one or more species of bacteria listed in Table 5. In some embodiments, the consortium of bacteria comprises the species listed in Table 5. In some embodiments, the consortium of bacteria comprises at least one bacterial species that consumes CH₄ and fixes N₂.

In some embodiments, the consortium of bacteria further comprises one or more additives. In some embodiments, the one or more additives improves viability of the consortium of bacteria. In some embodiments, the one or more additives are in a sufficient amount to keep at least one bacterial species of the consortium of bacteria capable of consuming CH₄ at a population frequency of at least 0.01%, 0.1%, or 1% for at least about 4 weeks. In some embodiments, the one or more additives increase CH₄ consumption or N₂ fixation as compared to a reference composition comprising the consortium of bacteria and lacking the one or more additives. In some embodiments, the one or more additives are present in an amount sufficient to favor consumption of the CH₄ by the consortium of bacteria over consumption of another organic molecule. In some embodiments, the one or more additive is present in the consortium of bacteria at a concentration of at least about 1 ug/L, about 10 ug/L, about 100 ug/L, or about 1 mg/L. In some embodiments, the one or more additives comprises at least one non-metal additive. In some embodiments, the at least one non-metal additive comprises phosphorus, ethylenediaminetetraacetic acid, or a combination thereof. In some embodiments, the one or more additives comprises at least one metal additive. In some embodiments, the one or more additives comprises at least one metallic salt additive. In some embodiments, the at least one metal additive comprises copper, tungsten, iron, nickel, molybdenum, cerium, manganese, lanthanum, zinc, cobalt, boron, or a combination thereof. In some embodiments, the one or more additives comprises copper.

In some embodiments, the consortium of bacteria comprises a bacterial species that expresses methane monooxygenase. In some embodiments, the consortium of bacteria comprises a bacterial species that expresses methyl coenzyme M reductase. In some embodiments, CH₄ consumption is determined by measuring a concentration of CH₄. In some embodiments, the concentration of CH₄ is determined using labeled CH₄. In some embodiments, the concentration of CH₄ is determined using radiolabeled CH₄. In some embodiments, the radiolabeled CH₄ comprises ³H-CH₄. In some embodiments, the radiolabeled CH₄ comprises ¹⁴C-CH₄. In some embodiments, the radiolabeled CH₄ comprises ¹³C-CH₄. In some embodiments, CH₄ consumption is determined by measuring a concentration of CO₂. In some embodiments, CH₄ consumption is determined by measuring a concentration of H₂O. In some embodiments, CH₄ consumption is determined by gas chromatography. In some embodiments, the gas chromatography comprises flame ionization detection gas chromatography. In some embodiments, CH₄ consumption is determined by a methane detector. In some embodiments, CH₄ consumption is determined by a portable methane detector.

In some embodiments, the consortium of bacteria increases N₂ fixation in low oxygen conditions. In some embodiments, the consortium of bacteria comprises at least one bacterial species that expresses nitrogenase. In some embodiments, N₂ fixation is determined by measuring a concentration of N₂. In some embodiments, the concentration of N₂ is determined using labeled N₂. In some embodiments, the concentration of N₂ is determined using radiolabeled N₂. In some embodiments, the radiolabeled N₂ comprises ¹³N-N₂. In some embodiments, the radiolabeled N₂ comprises ¹⁵N-N₂. In some embodiments, N₂ fixation is determined by measuring a concentration of NH₃. In some embodiments, N₂ fixation is determined by measuring a concentration of NH₄. In some embodiments, N₂ fixation is determined by measuring a concentration of CH₄. In some embodiments, N₂ fixation is determined by measuring a concentration of C₂H₄. In some embodiments, N₂ fixation is determined by gas chromatography. In some embodiments, the gas chromatography comprises flame ionization detection gas chromatography. In some embodiments, N₂ fixation is determined by an acetylene reduction assay. In some embodiments, N₂ fixation is determined by measuring a concentration of total nitrogen. In some embodiments, N₂ fixation is determined by combustion. In some embodiments, N₂ fixation is determined using a Dumas combustion method.

In some embodiments, the consortium of bacteria has viability for at least about 4 months or at least about 6 months. In some embodiments, the consortium of bacteria has viability for at least about 12 months. In some embodiments, the consortium of bacteria is stored at or about room temperature. In some embodiments, the consortium of is stored at or about 4° C. In some embodiments, the consortium of bacteria is stored at or about −20° C. In some embodiments, the consortium of bacteria comprises a neutral pH. In some embodiments, the consortium of bacteria comprises a basic pH. In some embodiments, the consortium of bacteria comprises an acidic pH. In some embodiments, the consortium of bacteria is freeze dried. In some embodiments, the consortium of bacteria is spray dried. In some embodiments, the consortium of bacteria is vacuum sealed.

In some embodiments, the consortium of bacteria disclosed herein comprises a kit. In some embodiments, a kit comprises the consortium of bacteria disclosed herein and instructions for use.

In some embodiments, the consortium of bacteria disclosed herein comprises a compost. In some embodiments, a compost comprises the consortium of bacteria. In some embodiments, the compost further comprises biomass. In some embodiments, the compost is enhanced with nitrogen compared to a reference compost that is substantially identical to the compost except the reference compost does not comprise the consortium of bacteria. In some embodiments, the compost improves nitrogen availability of soil when applied to the soil.

In some embodiments, the consortium of bacteria disclosed herein comprises a method for treating a crop. In some embodiments, the method for treating a crop comprises applying to the crop, to soil in which the crop is grown, or to liquid medium used to grow the crop the composition of the consortium of bacteria disclosed herein. In some embodiments, the crop is an agricultural crop. In some embodiments, the crop comprises one or more plants.

In some embodiments, the consortium of bacteria disclosed herein comprises use in a bioreactor. In some embodiments, a bioreactor comprises the composition of bacteria as disclosed herein.

In some embodiments, the consortium of bacteria disclosed herein comprises use in a system. In some embodiments, a system comprises a reaction chamber, a first fluid input in fluid communication with the reaction chamber, wherein the first fluid input is configured to direct gas enriched in methane (CH₄) into the reaction chamber, a second fluid input in fluid communication with the reaction chamber, wherein the second fluid input is configured to direct the consortium of bacteria as disclosed herein into the reaction chamber, and an outlet in fluid communication with the reaction chamber, wherein the enriched CH₄ is at a concentration of at least 1.89 parts per million (ppm). In some embodiments, the enriched CH₄ is from a storage of concentrated CH₄. In some embodiments, the enriched CH₄ is from an animal housing or free-range ruminants. In some embodiments, the enriched CH₄ is from any industrial source. In some embodiments, the industrial source comprises a natural gas pipeline, a syngas reactor, a reactor, or a refinery. In some embodiments, the enriched CH₄ is from any natural source. In some embodiments, the first fluid input and the second fluid input are the same input. In some embodiments, the first fluid input and the second fluid input are different from one another.

Disclosed herein is a composition comprising a consortium of bacteria. In some embodiments, the consortium of bacteria is capable of fixing nitrogen (N₂) and consuming methane (CH₄), wherein the consortium has at least one bacterial species from Table 1A or Table 3.

In some embodiments, the consortium of bacteria consumes methane (CH₄) at a rate greater than 5 umol/gDW per day. In some embodiments, the consortium of bacteria consumes methane (CH₄) at a rate greater than 13.5 umol/gDW per day. In some embodiments, the consortium of bacteria fixes nitrogen (N₂) at a rate greater than 2.0 umol/gDW per day. In some embodiments, the consortium of bacteria fixes nitrogen (N₂) at a rate greater than 2.5 umol/gDW per day.

In some embodiments, a first bacterium in the consortium of bacteria is unaffected by a second bacterium in the consortium of bacteria in respect to the first bacterium's ability to fix nitrogen. In some embodiments, a first bacterium in the consortium of bacteria is unaffected by a second bacterium in the consortium of bacteria in respect to the first bacterium's ability to consume CH₄.

In some embodiments, the consortium of bacteria comprises at least 1, 2, 3, 4, or more different bacterial phyla. In some embodiments, the consortium of bacteria comprises Micrarchaeota, Actinobacteria, Bacteroidetes, Firmicutes, Proteobacteria, or a combination thereof. In some embodiments, the consortium of bacteria comprises at least 1, 2, 3, 4, 5, 6, 7, 8, 9, 10, 15, 20, 30, or more different bacterial order. In some embodiments, the consortium of bacteria comprises Rhizobiales, Methylococcales, Methanobacteriales, Methanosarcinales, Nitrosomonadales, Pseudomonadale, or a combination thereof. In some embodiments, the consortium of bacteria comprises bacteria from at least 1, 2, 3, 4, 5, 6, 7, 8, 9, 10, 15, 20, 30, 40, 50, 60 or more different bacterial family. In some embodiments, the consortium of bacteria comprises Methylocystaceae, Methylococcaceae, Methanobacteriaceae, Methanosarcinaceae, Methylophilaceae, Pseudonocardiaceae, or a combination thereof. In some embodiments, the consortium of bacteria comprises bacteria from at least 1, 2, 3, 4, 5, 6, 7, 8, 9, 10, 15, 20, 30, 40, 50 or more different bacterial genera. In some embodiments, the consortium of bacteria comprises bacteria from at least 1, 2, 3, 4, 5, 6, 7, 8, 9, 10, 15, 20, 30, 40, 50 or more different species.

In some embodiments, the consortium of bacteria comprises species that are methanotrophs, nitrogen fixing bacteria, or both. In some embodiments, the consortium of bacteria comprises species from Tables 1, 3, or 4. In some embodiments, the consortium of bacteria comprises at least one or more species of bacteria listed in Table 5. In some embodiments, the consortium of bacteria comprises the species listed in Table 5. In some embodiments, the consortium of bacteria comprises at least one bacterial species that consumes CH₄ and fixes N₂.

In some embodiments, the consortium of bacteria further comprises one or more additives. In some embodiments, the one or more additives improves viability of the consortium of bacteria. In some embodiments, the one or more additives are in a sufficient amount to keep at least one bacterial species of the consortium of bacteria capable of consuming CH₄ at a population frequency of at least 0.01%, 0.1%, or 1% for at least about 4 weeks. In some embodiments, the one or more additives increase CH₄ consumption or N₂ fixation as compared to a reference composition comprising the consortium of bacteria and lacking the one or more additives. In some embodiments, the one or more additives are present in an amount sufficient to favor consumption of the CH₄ by the consortium of bacteria over consumption of another organic molecule. In some embodiments, the one or more additive is present in the consortium of bacteria at a concentration of at least about 1 ug/L, about 10 ug/L, about 100 ug/L, or about 1 mg/L. In some embodiments, the one or more additives comprises at least one non-metal additive. In some embodiments, the at least one non-metal additive comprises phosphorus, ethylenediaminetetraacetic acid, or a combination thereof. In some embodiments, the one or more additives comprises at least one metal additive. In some embodiments, the one or more additives comprises at least one metallic salt additive. In some embodiments, the at least one metal additive comprises copper, tungsten, iron, nickel, molybdenum, cerium, manganese, lanthanum, io zinc, cobalt, boron, or a combination thereof. In some embodiments, the one or more additives comprises copper.

In some embodiments, the consortium of bacteria comprises a bacterial species that expresses methane monooxygenase. In some embodiments, the consortium of bacteria comprises a bacterial species that expresses methyl coenzyme M reductase. In some embodiments, CH₄ consumption is determined by measuring a concentration of CH₄. In some embodiments, the concentration of CH₄ is determined using labeled CH₄. In some embodiments, the concentration of CH₄ is determined using radiolabeled CH₄. In some embodiments, the radiolabeled CH₄ comprises ³H-CH₄. In some embodiments, the radiolabeled CH₄ comprises ¹⁴C-CH₄. In some embodiments, the radiolabeled CH₄ comprises ¹³C-CH₄. In some embodiments, CH₄ consumption is determined by measuring a concentration of CO₂. In some embodiments, CH₄ consumption is determined by measuring a concentration of H₂O. In some embodiments, CH₄ consumption is determined by gas chromatography. In some embodiments, the gas chromatography comprises flame ionization detection gas chromatography. In some embodiments, CH₄ consumption is determined by a methane detector. In some embodiments, CH₄ consumption is determined by a portable methane detector.

In some embodiments, the consortium of bacteria increases N₂ fixation in low oxygen conditions. In some embodiments, the consortium of bacteria comprises at least one bacterial species that expresses nitrogenase. In some embodiments, N₂ fixation is determined by measuring a concentration of N₂. In some embodiments, the concentration of N₂ is determined using labeled N₂. In some embodiments, the concentration of N₂ is determined using radiolabeled N₂. In some embodiments, the radiolabeled N₂ comprises ¹³N-N₂. In some embodiments, the radiolabeled N₂ comprises ¹⁵N-N₂. In some embodiments, N₂ fixation is determined by measuring a concentration of NH₃. In some embodiments, N₂ fixation is determined by measuring a concentration of NH₄. In some embodiments, N₂ fixation is determined by measuring a concentration of CH₄. In some embodiments, N₂ fixation is determined by measuring a concentration of C₂H₄. In some embodiments, N₂ fixation is determined by gas chromatography. In some embodiments, the gas chromatography comprises flame ionization detection gas chromatography. In some embodiments, N₂ fixation is determined by an acetylene reduction assay. In some embodiments, N₂ fixation is determined by measuring a concentration of total nitrogen. In some embodiments, N₂ fixation is determined by combustion. In some embodiments, N₂ fixation is determined using a Dumas combustion method.

In some embodiments, the consortium of bacteria has viability for at least about 4 months or at least about 6 months. In some embodiments, the consortium of bacteria has viability for at least about 12 months. In some embodiments, the consortium of bacteria is stored at or about room temperature. In some embodiments, the consortium of is stored at or about 4° C. In some embodiments, the consortium of bacteria is stored at or about −20° C. In some embodiments, the consortium of bacteria comprises a neutral pH. In some embodiments, the consortium of bacteria comprises a basic pH. In some embodiments, the consortium of bacteria comprises an acidic pH. In some embodiments, the consortium of bacteria is freeze dried. In some embodiments, the consortium of bacteria is spray dried. In some embodiments, the consortium of bacteria is vacuum sealed.

In some embodiments, the consortium of bacteria disclosed herein comprises a kit. In some embodiments, a kit comprises the consortium of bacteria disclosed herein and instructions for use.

In some embodiments, the consortium of bacteria disclosed herein comprises a compost. In some embodiments, a compost comprises the consortium of bacteria. In some embodiments, the compost further comprises biomass. In some embodiments, the compost is enhanced with nitrogen compared to a reference compost that is substantially identical to the compost except the reference compost does not comprise the consortium of bacteria. In some embodiments, the compost improves nitrogen availability of soil when applied to the soil.

In some embodiments, the consortium of bacteria disclosed herein comprises a method for treating a crop. In some embodiments, the method for treating a crop comprises applying to the crop, to soil in which the crop is grown, or to liquid medium used to grow the crop the composition of the consortium of bacteria disclosed herein. In some embodiments, the crop is an agricultural crop. In some embodiments, the crop comprises one or more plants.

In some embodiments, the consortium of bacteria disclosed herein comprises use in a bioreactor. In some embodiments, a bioreactor comprises the composition of bacteria as disclosed herein.

In some embodiments, the consortium of bacteria disclosed herein comprises use in a system. In some embodiments, a system comprises a reaction chamber, a first fluid input in fluid communication with the reaction chamber, wherein the first fluid input is configured to direct gas enriched in methane (CH₄) into the reaction chamber, a second fluid input in fluid communication with the reaction chamber, wherein the second fluid input is configured to direct the consortium of bacteria as disclosed herein into the reaction chamber, and an outlet in fluid communication with the reaction chamber, wherein the enriched CH₄ is at a concentration of at least 1.89 parts per million (ppm). In some embodiments, the enriched CH₄ is from a storage of concentrated CH₄. In some embodiments, the enriched CH₄ is from an animal housing or free-range ruminants. In some embodiments, the enriched CH₄ is from any industrial source. In some embodiments, the industrial source comprises a natural gas pipeline, a syngas reactor, a reactor, or a refinery. In some embodiments, the enriched CH₄ is from any natural source. In some embodiments, the first fluid input and the second fluid input are the same input. In some embodiments, the first fluid input and the second fluid input are different from one another.

Disclosed herein is a method of enriching for one or more bacteria. In some embodiments, the method comprises introducing to a composition a consortium of bacteria comprising the one or more bacteria and an additive comprising a metal, wherein the consortium of bacteria consumes methane (CH₄) at a rate of at least 5 umol/gDW per day.

In some embodiments, the method enriches for a consortium of bacteria that is capable of consuming methane (CH₄) at a rate greater than 13.5 umol/gDW per day and fixing nitrogen (N₂) at a rate greater than 2.0 umol/gDW per day. In some embodiments, the consortium of bacteria is capable of consuming methane (CH₄) at a rate greater than 5 umol/gDW per day and fixing nitrogen (N₂) at a rate greater than 2.5 umol/gDW per day. In some embodiments, the method enriches for a consortium of bacteria that is capable of fixing nitrogen (N₂) and consuming methane (CH₄), wherein the consortium of bacteria has viability for at least about 3 months. In some embodiments, the method enriches for a consortium of bacteria that is capable of fixing nitrogen (N₂) and consuming methane (CH₄), wherein the consortium has at least one bacterial species from Table 4. In some embodiments, the method enriches for a consortium of bacteria that is capable of fixing nitrogen (N₂) and consuming methane (CH₄), wherein the consortium has at least one bacterial species from Table 1A, Table 1C, or Table 3.

In some embodiments, the metal comprises molybdenum, copper, tungsten, zinc, manganese, cobalt, nickel, boron, iron, cerium, lanthanum, or a combination thereof. In some embodiments, the metal comprises copper. In some embodiments, the metal is present in the consortium of bacteria at a concentration of at least about 1 ug/L, about 10 ug/L, about 100 ug/L, or about 1 mg/L. In some embodiments, the additive further comprises magnesium sulfate heptahydrate (MgSO₄·7H₂O), potassium nitrate (KNO₃), calcium chloride dihydrate (CaCl₂·2H₂O), monopotassium phosphate (KH₂PO₄), dibasic potassium phosphate (K₂HPO₄), sodium molybdate dihydrate (Na₂MoO₄ 2H₂O), copper(II) sulfate pentahydrate (CuSO₄·5H₂O), zinc sulfate heptahydrate (ZnSO₄·7H₂O), manganese(II) chloride tetrahydrate (MnCl₂·4H₂O), cobalt(II) chloride hexahydrate (CoCl₂·6H₂O), nickel(II) chloride hexahydrate (NiCl₂·6H₂O), boric acid (H₃BO₃), ethylenediaminetetraacetic acid (EDTA), ethylenediaminetetraacetic acid ferric sodium salt, or a combination thereof.

In some embodiments, the one or more bacteria are increased by at least about 10%, 20%, 30%, 40% or 50% as compared to a reference composition that comprises the consortium of bacteria and lacking the additive, one month, two months, four months, or six months after the introducing. In some embodiments, the one or more bacteria comprises a methanotroph. In some embodiments, the one or more bacteria comprises a species listed in Table 1A. In some embodiments, the one or more bacteria comprises a nitrogen fixing bacteria. In some embodiments, the one or more bacteria comprises a species listed in Table 3. In some embodiments, the one or more bacteria comprises a bacteria that is capable of consuming methane and fixing nitrogen. In some embodiments, the one or more bacteria comprises a species listed in Table 4.

In some embodiments, the composition is a liquid composition. In some embodiments, the composition comprises soil. In some embodiments, the composition comprises compost. In some embodiments, an undesirable bacterium that is present in the composition prior to the introducing is undetectable about 3 months following the introducing. In some embodiments, an undesirable bacterium that is present in the composition prior to the introducing is undetectable about 5 months following the introducing. In some embodiments, an undesirable virus that is present in the composition prior to the introducing is undetectable about 3 months following the introducing. In some embodiments, following the introducing, the composition is exposed to ambient air.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth nonlimiting illustrative embodiments, in which the principles of the disclosure are utilized, and the accompanying drawings of which:

FIG. 1 depicts schematic of a compost reactor.

FIGS. 2A-2B depict schematic of a trickling biofilm reactor. FIG. 2A depicts a schematic of a trickling biofilm reactor with venturi. FIG. 2B depicts a schematic of a trickling biofilm reactor with recycle.

FIG. 3 depicts species distribution in control compost and outdoor compost at various time points.

FIGS. 4A-4D depict the distribution of bacteria in liquid cultures and biofilm at various time points from metagenomic analyses. FIG. 4A depicts phylum distribution of bacteria present in liquid cultures and biofilm at various time points. FIG. 4B depicts order distribution of bacteria present in liquid cultures and biofilm at various time points. FIG. 4C depicts family distribution of bacteria present in liquid cultures and biofilm at various time points. FIG. 4D depicts species distribution of bacteria present in liquid cultures and biofilm at various time points.

FIG. 5 depicts an image of a fermentation tank.

FIG. 6 depicts a graph of the optical density (OD) over time (hours) for a sample containing a bacterial consortium. The first line of the double lines indicates time points where samples were collected for sample processing. The second line indicates the end of sample processing for a given time point.

FIG. 7 depicts the traces of material added (NaNO₃ (mg/ml), total acid (ml)), optical density (OD), and dissolved oxygen (pO2) during the course of a bacterial growth experiment.

FIG. 8 depicts concentration traces for different elements during the course of a bacterial growth experiment.

FIG. 9 depicts the genus distribution of a bacteria sample in liquid cultures grown in outdoor ambient conditions and indoor laboratory conditions in serum bottles across different time points.

FIG. 10 depicts the species relative abundance of two batches of a bacterial consortium grown from different aliquots.

FIG. 11 depicts the genus distribution of a bacteria sample in liquid cultures grown in outdoor ambient conditions and indoor laboratory conditions in serum bottles across different time points.

FIG. 12 depicts the species relative abundance of two batches of a bacterial consortium grown from different aliquots.

FIG. 13 depicts a schematic of a solid-state bioreactor.

FIG. 14 depicts an image of a physical property testing apparatus.

FIG. 15 depicts the results of a bulk density, total porosity and field capacity analysis of columns made with biochar, perlite and fine biochar.

FIG. 16 depicts moisture content analysis over time of columns made with biochar, perlite and fine biochar.

FIG. 17 depicts the size distribution of the matrices used in columns generated with biochar and perlite.

FIG. 18 depicts water loss and saturation level measurements taken at different temperatures and with different flowrates for a moisture saturator attached to a feed gas port as part of a bioreactor.

FIG. 19 depicts the methane consumption rate as a function of time of bacterial consortium samples housed within columns containing Perlite at different concentrations.

FIG. 20 depicts the methane consumption rate as a function of time of bacterial consortium samples housed within columns containing Biochar at different concentrations.

FIG. 21 depicts the methane consumption rate as a function of time of bacterial consortium samples housed within columns containing Perlite at different concentrations.

FIG. 22 depicts the methane consumption rate as a function of time of bacterial consortium samples housed within columns containing Biochar at different concentrations.

FIG. 23 depicts experimental conditions for testing bacterial consortium methane consumption rates within columns containing a matrix material.

FIG. 24 depicts experimental conditions for testing bacterial consortium methane consumption rates within columns containing a matrix material.

FIG. 25 depicts genus distribution of bacteria consortium samples grown with Biochar within a bioreactor.

FIG. 26 depicts genus distribution of bacteria consortium samples grown with Perlite within a bioreactor.

### DETAILED DESCRIPTION

Provided herein is disclosure related to a consortium of bacteria. More specifically, the disclosure provides consortiums of bacteria that are capable of consuming methane and fixing nitrogen. In some aspects, the consortium of bacteria is capable of consuming methane or fixing nitrogen.

### I. Methanotrophs

Methanotrophs metabolize methane as a sole carbon and energy source or as one of different carbon and energy sources. Methanotrophs can be found in a wide range of environments and are characterized by an ability to consume methane, for example oxidizing methane into methanol and formaldehyde, followed by either cellular anabolism or further oxidation to formic acid and carbon dioxide for energy metabolism. The methane oxidation of methanotrophs is often mediated by methane monooxygenases, a class of enzymes capable of oxidizing the C-H bonds in methane. Methanotrophs play a large role in consuming atmospheric methane and participate in ecosystems by acting as soil sinks for atmospheric carbon as well as consuming methane emissions from soils and other natural sources before they can reach the atmosphere.

In some embodiments, a consortium of bacteria described herein is capable of consuming methane. In some embodiments, the consortium of bacteria comprises at least one bacterium that is capable of consuming methane. In some embodiments, a bacterium that is capable of consuming methane may be capable of using single-carbon compounds, such as methanol or formate. In some embodiments, a species that is capable of consuming methane may be capable of using multi-carbon compounds, such as a range of hydrocarbons or chlorinated hydrocarbons. In some embodiments, methanotrophs are listed in Table 1A, Table 1B, Table 1C, Table 1D, or a combination thereof.

In certain embodiments, a bacteria that consumes methane may oxidize the methane. In aerobic conditions, a bacterium capable of oxidizing methane may oxidize CH₄ with 02 into methanol and formaldehyde. The formaldehyde may be incorporated into organic compounds via a RuMP pathway (type I methanotroph) or a serine pathway (type II methanotroph). In aerobic conditions, the complete methane oxidation pathway may oxidize CH₄ with 02 into CO₂ and H₂O. In some embodiments, the oxidation of CH₄ is catalyzed by a methane monooxygenase (MMO) enzyme. In certain embodiments, the MMO may comprise particulate methane monooxygenase (pMMO) or soluble methane monooxygenase (sMMO). In various embodiments, the methane monooxygenase may comprise variants of MMO, nonlimiting examples of which include PmoCBA or MmoXYZCBG.

In certain embodiments, a bacteria may oxidize methane in anaerobic conditions. In combination embodiments, a bacteria may oxidize methane in anaerobic conditions when associated with archaea. In some embodiments, a bacteria may express methyl coenzyme A reductase. In some embodiments, the expression of methyl coenzyme A reductase may enable a bacteria to oxidize methane. In some embodiments, methyl coenzyme A reductase may oxidize methane in anaerobic conditions. In some embodiments a bacteria may oxidize methane using an intra-aerobic pathway. In some embodiments, a bacteria may oxidize methane by dismutating nitrite into oxide and free oxygen. In some embodiments, the oxygen may oxidize methane. A combination of archaea and bacteria can carry out nitrate- and nitrite-dependent methane oxidation. In some embodiments an archaea or bacteria can respire methane with nitrite via the dismutation of nitric oxide into nitrogen and free oxygen. In some embodiments, said oxygen may oxidize methane. In some embodiments, archaea or bacteria can respire methane with nitrate by reverse methanogenesis via the Wood-Ljungdhal pathway.

In some embodiments, the consortium of bacteria comprises at least one or more species that is capable of consuming methane. In some embodiments, the bacteria that express methane monooxygenase are phylogenetically diverse. In some embodiments, the consortium of bacteria comprises at least 1, 2, 3, 4, or more different bacterial phyla. In certain embodiments, a bacteria capable of consuming methane may belong to the phyla Micrarchaeota, Actinobacteria, Bacteroidetes, Firmicutes, Pseudomonadota, or Proteobacteria. In certain embodiments, the consortium of bacteria may comprise at least 1, 2, 3, 4, 5, 6, 7, 8, 9, 10, 15, 20, 30, or more different bacterial order. In certain embodiments, a bacteria capable of consuming methane may belong to the order Rhizobiales, Methylococcales, Methanobacteriales, Methanosarcinales, Nitrosomonadales, or Pseudomonadale. In certain embodiments, the consortium of bacteria may comprise at least 1, 2, 3, 4, 5, 6, 7, 8, 9, 10, 15, 20, 30, 40, 50, or more different bacterial family. In certain embodiments, a bacteria capable of consuming methane may belong to the family Methylocystaceae, Methylococcaceae, Methanobacteriaceae, Methanosarcinaceae, Methylophilaceae or Pseudonocardiaceae. In certain embodiments, the consortium of bacteria may comprise at least 1, 2, 3, 4, 5, 6, 7, 8, 9, 10, 15, 20, 30, 40, 50, or more different bacterial genera. In certain embodiments, a bacteria capable of consuming methane may belong to the genera Methylococcus, Methylomonas, Methylomicrobium, Methylobacter, Methylocaldum, Methylovulum, Methylomarinum, Methylomarinovum, Methylothermus, Methylocystis, Methylosinus, or Pseudomonas. In some embodiments, the consortium of bacteria may comprise 1, 2, 3, 4, 5, 6, 7, 8, 9, 10, 11, 12, 13, 14, 15, 16, 17, 18, 19, 20, 25, 30, 35, 40, 45, 50, 55, 60, or more species that are capable of consuming methane. In some embodiments, the consortium of bacteria may comprise 1, 2, 3, 4, 5, 6, 7, 8, 9, 10, 11, 12, 13, 14, 15, 16, 17, 18, 19, 20, 25, 30, 35, 40, 45, 50, 55, 60, or more strains that are capable of consuming methane.

In some embodiments, the consortium of bacteria may change over time. In some embodiments, the phyla distribution, family distribution, genus distribution, species distribution, strain distribution, or a combination thereof of the bacteria consortium may change over time. In some embodiments, the consortium of bacteria may not change over time. In some embodiments, the phyla distribution, family distribution, genus distribution, species distribution, strain distribution, or a combination thereof of the bacteria consortium may not change over time.

In some embodiments, the consortium of bacteria may comprise a species from Table 1A, Table 1C, or a combination thereof. In some embodiments, the consortium of bacteria may comprise 1, 2, 3, 4, 5, 6, 7, 8, 9, 10, or more species from Table 1A, Table 1C, or a combination thereof. In certain embodiments, the consortium of bacteria may comprise one or more species from Table 1A, Table 1C, or a combination thereof in combination with other bacteria that may consume methane only, fix nitrogen only, consume methane and fix nitrogen, or do neither consume methane nor fix nitrogen. In various embodiments, the consortium of bacteria may comprise only bacteria listed in Table 1A. In some embodiments, the consortium of bacteria may comprise only bacteria listed in Table 1C.

In some embodiments, the consortium of bacteria may comprise a genus from Table 1B. In some embodiments, the consortium of bacteria may comprise 1, 2, 3, 4, 5, 6, 7, 8, 9, 10, or more genera from Table 1B. In certain embodiments, the consortium of bacteria may comprise one or more species from Table 1B in combination with other bacteria that may consume methane only, fix nitrogen only, consume methane and fix nitrogen, or do neither consume methane nor fix nitrogen. In various embodiments, the consortium of bacteria may comprise only bacteria listed in Table 1B.

In some embodiments, the consortium of bacteria may comprise a strain from Table 1D. In some embodiments, the consortium of bacteria may comprise 1, 2, 3, 4, 5, 6, 7, 8, 9, 10, or more strains from Table 1D. In certain embodiments, the consortium of bacteria may comprise one or more strains from Table 1D in combination with other bacteria that may consume methane only, fix nitrogen only, consume methane and fix nitrogen, or do neither consume methane nor fix nitrogen. In various embodiments, the consortium of bacteria may comprise only bacteria listed in Table 1D.

In some embodiments, one bacteria phyla of the consortium of bacteria may comprise 0.000001-99.999%, about 0.00001-99.99%, about 0.0001-99.9%, about 0.001-99%, about 0.01-90%, about 0.1-80%, about 1-70%, about 2-60%, about 3-50%, about 4-40%, about 5-30%, about 6-25%, about 7-20%, about 8-15%, about 9-10%, about 0.000001%, about 0.00001%, about 0.0001%, about 0.001%, about 0.01%, about 0.1%, about 1%, about 2%, about 3%, about 4%, about 5%, about 6%, about 7%, about 8%, about 9%, about 10%, about 15%, about 20%, about 25%, about 30%, about 40%, about 50%, about 60%, about 70%, about 80%, about 90%, about 99%, about 99.9%, about 99.99%, or about 99.999% of the total consortium of bacteria.

In some embodiments, one bacteria family of the consortium of bacteria may comprise 0.000001-99.999%, about 0.00001-99.99%, about 0.0001-99.9%, about 0.001-99%, about 0.01-90%, about 0.1-80%, about 1-70%, about 2-60%, about 3-50%, about 4-40%, about 5-30%, about 6-25%, about 7-20%, about 8-15%, about 9-10%, about 0.000001%, about 0.00001%, about 0.0001%, about 0.001%, about 0.01%, about 0.1%, about 1%, about 2%, about 3%, about 4%, about 5%, about 6%, about 7%, about 8%, about 9%, about 10%, about 15%, about 20%, about 25%, about 30%, about 40%, about 50%, about 60%, about 70%, about 80%, about 90%, about 99%, about 99.9%, about 99.99%, or about 99.999% of the total consortium of bacteria.

In some embodiments, one bacteria genus of the consortium of bacteria may comprise 0.000001-99.999%, about 0.00001-99.99%, about 0.0001-99.9%, about 0.001-99%, about 0.01-90%, about 0.1-80%, about 1-70%, about 2-60%, about 3-50%, about 4-40%, about 5-30%, about 6-25%, about 7-20%, about 8-15%, about 9-10%, about 0.000001%, about 0.00001%, about 0.0001%, about 0.001%, about 0.01%, about 0.1%, about 1%, about 2%, about 3%, about 4%, about 5%, about 6%, about 7%, about 8%, about 9%, about 10%, about 15%, about 20%, about 25%, about 30%, about 40%, about 50%, about 60%, about 70%, about 80%, about 90%, about 99%, about 99.9%, about 99.99%, or about 99.999% of the total consortium of bacteria.

In some embodiments, one bacteria species of the consortium of bacteria may comprise 0.000001-99.999%, about 0.00001-99.99%, about 0.0001-99.9%, about 0.001-99%, about 0.01-90%, about 0.1-80%, about 1-70%, about 2-60%, about 3-50%, about 4-40%, about 5-30%, about 6-25%, about 7-20%, about 8-15%, about 9-10%, about 0.000001%, about 0.00001%, about 0.0001%, about 0.001%, about 0.01%, about 0.1%, about 1%, about 2%, about 3%, about 4%, about 5%, about 6%, about 7%, about 8%, about 9%, about 10%, about 15%, about 20%, about 25%, about 30%, about 40%, about 50%, about 60%, about 70%, about 80%, about 90%, about 99%, about 99.9%, about 99.99%, or about 99.999% of the total consortium of bacteria.

In some embodiments, one bacteria strain of the consortium of bacteria may comprise 0.000001-99.999%, about 0.00001-99.99%, about 0.0001-99.9%, about 0.001-99%, about 0.01-90%, about 0.1-80%, about 1-70%, about 2-60%, about 3-50%, about 4-40%, about 5-30%, about 6-25%, about 7-20%, about 8-15%, about 9-10%, about 0.000001%, about 0.00001%, about 0.0001%, about 0.001%, about 0.01%, about 0.1%, about 1%, about 2%, about 3%, about 4%, about 5%, about 6%, about 7%, about 8%, about 9%, about 10%, about 15%, about 20%, about 25%, about 30%, about 40%, about 50%, about 60%, about 70%, about 80%, about 90%, about 99%, about 99.9%, about 99.99%, or about 99.999% of the total consortium of bacteria.

TABLE 1A
Species containing MMO gene homologs

Species
Acetanaerobacterium elongatum
Acetatifactor muris
Acetivibrio cellulolyticus
Acetivibrio clariflavus
Acetivibrio ethanolgignens
Acetivibrio saccincola
Acetoanaerobium noterae
Acetoanaerobium pronyense
Acetoanaerobium sticklandii
Acetobacterium bakii
Acetobacterium dehalogenans
Acetobacterium fimetarium
Acetobacterium malicum
Acetobacterium paludosum
Acetobacterium sp. KB-1
Acetobacterium tundrae
Acetobacterium wieringae
Acetobacterium woodii
Acetobacterium
Acetobacteroides hydrogenigenes
Acetohalobium arabaticum
Acetonema longum
Achromobacter aegrifaciens
Achromobacter agilis
Achromobacter denitrificans
Achromobacter insolitus
Acidaminobacter sp. JC074
Acidianus brierleyi
Acidicapsa acidisoli
Acidicapsa dinghuensis
Acidiferrobacter sp. SPIII_3
Acidiferrobacter thiooxydans
Acidihalobacter aeolianus
Acidihalobacter prosperus
Acidiphilium cryptum JF-5
Acidiphilium cryptum
Acidiphilium multivorum AIU301
Acidiphilium multivorum
Acidiphilium sp. 20-67-58
Acidiphilium sp. 21-68-69
Acidiphilium sp. 34-64-41
Acidiphilium sp. 37-64-53
Acidiphilium sp. 37-67-22
Acidiphilium sp. JA12-A1
Acidiphilium sp. PM
Acidiphilium
Acidisphaera rubrifaciens HS-AP3
Acidithiobacillus ferridurans
Acidithiobacillus ferriphilus
Acidithiobacillus ferrivorans SS3
Acidithiobacillus ferrivorans
Acidithiobacillus ferrooxidans ATCC 23270
Acidithiobacillus ferrooxidans ATCC 53993
Acidithiobacillus ferrooxidans
Acidithiobacillus sp. ‘AMD consortium’
Acidithiobacillus
Acidithrix ferrooxidans
Acidithrix sp. C25
Acidobacteriota bacterium
Acidovorax caeni
Acidovorax delafieldii
Acidovorax sp. JHL-3
Acidovorax sp. YS12
Acinetobacter baumannii
Acinetobacter haemolyticus
Acinetobacter pittii
Acinetobacter soli
Acinetobacter sp. 25977_3
Acinetobacter sp. ANC 4169
Acinetobacter sp. ANC 4779
Acinetobacter sp. PaPiEm2-1
Acinetobacter sp. PaPiHh2-2
Acinetobacter sp. V2
Actinacidiphila alni
Actinacidiphila bryophytorum
Actinacidiphila guanduensis
Actinacidiphila soli
Actinoallomurus bryophytorum
Actinobacteria bacterium 13_1_20CM_3_71_11
Actinocrispum wychmicini
Actinomadura viridilutea
Actinomycetospora callitridis
Actinomycetospora chiangmaiensis
Actinomycetospora chibensis
Actinomycetospora cinnamomea
Actinomycetospora corticicola
Actinomycetospora endophytica
Actinomycetospora lutea
Actinomycetospora soli
Actinomycetospora sp. DW7H6
Actinomycetospora sp. TBRC 11914
Actinomycetospora straminea
Actinomycetospora succinea
Actinomycetota bacterium
Actinophytocola oryzae
Actinoplanes atraurantiacus
Actinoplanes bogorensis
Actinoplanes brasiliensis
Actinoplanes campanulatus
Actinoplanes deccanensis
Actinoplanes derwentensis
Actinoplanes globisporus
Actinoplanes ianthinogenes
Actinoplanes italicus
Actinoplanes lichenicola
Actinoplanes lobatus
Actinoplanes lutulentus
Actinoplanes ovalisporus
Actinoplanes regularis
Actinoplanes sp. ATCC 53533
Actinoplanes sp. KI2
Actinoplanes sp. LAM7112
Actinoplanes sp. Pm04-4
Actinoplanes sp. TBRC 11911
Actinoplanes subtropicus
Actinoplanes tereljensis
Actinoplanes utahensis
Actinoplanes xinjiangensis
Actinorugispora endophytica
Acuticoccus sediminis
Aequitasia blattaphilus
Aerococcus urinae
Aeromonas allosaccharophila
Aeromonas caviae
Aeromonas dhakensis
Aeromonas finlandensis
Aeromonas hydrophila
Aeromonas jandaei
Aeromonas lacus
Aeromonas piscicola
Aeromonas simiae
Aeromonas sobria
Aeromonas sp. 1805
Aeromonas sp. A35_P
Aeromonas sp. BC14
Aeromonas sp. CU5
Aeromonas sp. FDAARGOS 1403
Aeromonas sp. FDAARGOS 1410
Aeromonas sp. HMWF016
Aeromonas sp. L_1B5_3
Aeromonas sp. QDB09
Aeromonas sp. QDB11
Aeromonas sp. RU39B
Aeromonas sp. S41-2
Aeromonas sp. SCS5
Aeromonas sp. SG16
Aeromonas sp. Y318-1
Aeromonas sp. YN13HZO-058
Aeromonas veronii
Aeromonas
Aerosticca soli
Aestuariicella hydrocarbonica
Afipia felis
Agreia sp. COWG
Agromyces ramosus
Albibacterium bauzanense
Alginatibacterium sediminis
Algoriphagus antarcticus
Algoriphagus aquaeductus
Algoriphagus boseongensis
Algoriphagus chordae
Algoriphagus marincola HL-49
Algoriphagus ratkowskyi
Algoriphagus yeomjeoni
Algoriphagus zhangzhouensis
Aliarcobacter butzleri
Aliarcobacter cibarius
Alicyclobacillus herbarius
Alicyclobacillus shizuokensis
Aliidiomarina quisquiliarum
Aliikangiella coralliicola
Aliiroseovarius pelagivivens
Alishewanella sp.
Alistipes sp. ZOR0009
Alkaliflexus imshenetskii
Alkaliflexus sp. Ai-910
Alkalilimnicola ehrlichii
Alkalimarinus sediminis
Alkalimarinus sp. A2M4
Alkaliphilus hydrothermalis
Alkaliphilus metalliredigens
Alkaliphilus oremlandii
Alkaliphilus peptidifermentans
Alkaliphilus pronyensis
Alkaliphilus serpentinus
Alkaliphilus sp. B6464
Alkaliphilus transvaalensis
Alkalispirochaeta alkalica
Alkalispirochaeta americana
Alkalispirochaeta sphaeroplastigenens
Alkalitalea saponilacus
Alloacidobacterium dinghuense
Allonocardiopsis opalescens
Alphaproteobacteria bacterium ADurb.BinA305
Altererythrobacter sp. XM-24bin4
Amaricoccus solimangrovi
Aminiphilus circumscriptus
Aminipila butyrica
Aminipila terrae
Aminivibrio pyruvatiphilus
Aminobacter sp. DSM 101952
Aminobacter sp. MSH 1
Ammoniphilus sp. YIM 78166
Amphritea atlantica
Amphritea balenae
Amphritea japonica
Amphritea pacifica
Amycolatopsis acidicola
Amycolatopsis acididurans
Amycolatopsis acidiphila
Amycolatopsis alba DSM 44262
Amycolatopsis alba
Amycolatopsis alkalitolerans
Amycolatopsis antarctica
Amycolatopsis australiensis
Amycolatopsis balhimycina DSM 5908
Amycolatopsis balhimycina
Amycolatopsis bartoniae
Amycolatopsis benzoatilytica
Amycolatopsis bullii
Amycolatopsis camponoti
Amycolatopsis circi
Amycolatopsis deserti
Amycolatopsis eburnea
Amycolatopsis echigonensis
Amycolatopsis jejuensis
Amycolatopsis kentuckyensis
Amycolatopsis lexingtonensis
Amycolatopsis mediterranei RB
Amycolatopsis mediterranei S699
Amycolatopsis mediterranei U32
Amycolatopsis mediterranei
Amycolatopsis methanolica 239
Amycolatopsis methanolica group
Amycolatopsis methanolica
Amycolatopsis niigatensis
Amycolatopsis orientalis
Amycolatopsis panacis
Amycolatopsis pithecellobii
Amycolatopsis pittospori
Amycolatopsis pretoriensis
Amycolatopsis rhizosphaerae
Amycolatopsis rifamycinica
Amycolatopsis rubida
Amycolatopsis saalfeldensis
Amycolatopsis sacchari
Amycolatopsis sp. AA4
Amycolatopsis sp. ATCC 39116
Amycolatopsis sp. BJA-103
Amycolatopsis sp. CA-126428
Amycolatopsis sp. CA-128772
Amycolatopsis sp. DSM 110486
Amycolatopsis sp. FDAARGOS 1241
Amycolatopsis sp. FU40
Amycolatopsis sp. GM8
Amycolatopsis sp. HUAS 11-8
Amycolatopsis sp. Hca4
Amycolatopsis sp. M39
Amycolatopsis sp. OK19-0408
Amycolatopsis sp. Poz14
Amycolatopsis sp. SID8362
Amycolatopsis sp. WAC 01375
Amycolatopsis sp. WAC 01416
Amycolatopsis sp. WQ 127309
Amycolatopsis sulphurea
Amycolatopsis taiwanensis
Amycolatopsis thermoflava
Amycolatopsis tolypomycina
Amycolatopsis vancoresmycina DSM 44592
Amycolatopsis vancoresmycina
Amycolatopsis vastitatis
Amycolatopsis xylanica
Amycolatopsis
Anabaena sp. UHCC 0204
Anabaena sp. UHCC 0253
Anaeroarcus burkinensis
Anaerobacterium chartisolvens
Anaerocolumna chitinilytica
Anaerocolumna jejuensis
Anaerocolumna xylanovorans
Anaerolinea thermolimosa
Anaeromicrobium sediminis
Anaeromyxobacter dehalogenans
Anaeromyxobacter diazotrophicus
Anaeromyxobacter oryzae
Anaeromyxobacter paludicola
Anaeromyxobacter sp. Fw109-5
Anaeromyxobacter sp. K
Anaeromyxobacter sp. PSR-1
Anaeromyxobacter sp. SG17
Anaeromyxobacter sp. SG22
Anaeromyxobacter sp. SG26
Anaeromyxobacter sp. SG66
Anaeromyxobacter
Anaeropeptidivorans aminofermentans
Anaerophaga thermohalophila
Anaerophilus nitritogenes
Anaerosalibacter massiliensis
Anaerospora hongkongensis
Anaerosporobacter mobilis
Anaerosporomusa subterranea
Anaerotignum neopropionicum
Anaerotignum propionicum
Anaerotruncus colihominis
Anaerovorax sp. IOR16
Ancylobacter aquaticus
Ancylobacter polymorphus
Ancylomarina sp. 16SWW S1-10-2
Aneurinibacillus danicus
Aneurinibacillus soli
Aneurinibacillus sp. B1
Aneurinibacillus tyrosinisolvens
Anseongella ginsenosidimutans
Antarctobacter heliothermus
Aquabacterium sp. CECT 9606
Aquabacterium sp. NJ1
Aquabacterium sp. OR-4
Aquicella lusitana
Archaeoglobus neptunius
Arcicella aurantiaca
Arcobacter acticola
Arcobacter aquimarinus
Arcobacter caeni
Arcobacter cloacae
Arcobacter defluvii
Arcobacter ellisii
Arcobacter peruensis
Arcobacter sp. ARW1-2F2
Arcobacter sp. CECT 9188
Arcobacter sp. F155
Arcobacter sp. L
Arcobacter suis
Arcobacter venerupis
Arcobacteraceae
Arcticibacter pallidicorallinus
Arcticibacter tournemirensis
Arenibacter algicola
Arenibacter echinorum
Arenibacter palladensis
Arenibacter sp. ARW7G5Y1
Arenibacter sp. NBRC 103722
Arenibaculum pallidiluteum
Aromatoleum diolicum
Aromatoleum evansii
Aromatoleum petrolei
Aromatoleum toluclasticum
Aromatoleum tolulyticum
Aromatoleum toluolicum
Aromatoleum toluvorans
Arthrobacter sp. AG258
Arthrobacter sp. AG367
Arthrobacter sp. Bi26
Arthrobacter sp. Hiyo4
Arthrobacter sp. MMS18-M83
Arthrobacter sp. SLBN-112
Arthrobacter sp. SLBN-122
Arthrobacter sp. zg-Y820
Asaccharospora irregularis
Asanoa ferruginea
Asanoa hainanensis
Asanoa iriomotensis
Asanoa ishikariensis
Asanoa siamensis
Aspergillus calidoustus
Aspergillus candidus
Aspergillus fischeri NRRL 181
Aspergillus fumigatus A1163
Aspergillus fumigatus var. RP-2014
Aspergillus fumigatus
Aspergillus luchuensis
Aspergillus novofumigatus IBT 16806
Aspergillus ochraceoroseus IBT 24754
Aspergillus ochraceoroseus
Aspergillus piperis CBS 112811
Aspergillus rambellii
Aurantimonas marina
Aurantimonas sp. LRZ36
Azoarcus communis
Azoarcus indigens
Azoarcus olearius
Azoarcus sp. Aa7
Azoarcus sp. DD4
Azoarcus sp. KH32C
Azoarcus sp. TTM-91
Azoarcus sp.
Azoarcus taiwanensis
Azonexus fungiphilus
Azonexus hydrophilus
Azonexus sp. R2A-61
Azospira inquinata
Azospira oryzae
Azospira restricta
Azospira sp. I09
Azospira sp. I13
Azospira
Azospirillum agricola
Azospirillum argentinense
Azospirillum baldaniorum
Azospirillum brasilense
Azospirillum canadense
Azospirillum cavernae
Azospirillum doebereinerae
Azospirillum fermentarium
Azospirillum formosense
Azospirillum griseum
Azospirillum halopraeferens
Azospirillum humicireducens
Azospirillum lipoferum
Azospirillum melinis
Azospirillum oleiclasticum
Azospirillum oryzae
Azospirillum palustre
Azospirillum picis
Azospirillum ramasamyi
Azospirillum rugosum
Azospirillum soli
Azospirillum sp. 412522
Azospirillum sp. A1-3
Azospirillum sp. B21
Azospirillum sp. B506
Azospirillum sp. B510
Azospirillum sp. INR13
Azospirillum sp. OGB3
Azospirillum sp. Sh1
Azospirillum sp. TSA2s
Azospirillum sp. TSA6c
Azospirillum sp. TSH100
Azospirillum sp. TSH20
Azospirillum sp. TSH58
Azospirillum sp. TSH64
Azospirillum sp. TSH7
Azospirillum sp. TSO22-1
Azospirillum sp. TSO35-2
Azospirillum sp. TSO5
Azospirillum sp. YIM B02556
Azospirillum sp. YIM DDC1
Azospirillum tabaci
Azospirillum thermophilum
Azospirillum thiophilum
Azospirillum
Azovibrio restrictus
Bacillus benzoevorans
Bacillus rubiinfantis
Bacillus smithii
Bacillus sp. 1NLA3E
Bacillus sp. EB600
Bacillus sp. PK3_68
Bacillus sp. V3B
Bacillus tuaregi
Bacteria
Bacteriovorax sp. BAL6_X
Bacteriovorax sp. BSW11_IV
Bacteriovorax sp. DB6_IX
Bacteriovorax sp. Seq25_V
Bacteriovorax stolpii
Baekduia sp. 0141_2
Bathymodiolus brooksi gill symbiont
Bathymodiolus heckerae gill symbiont
Bathymodiolus japonicus methanotrophic gill symbiont
Bathymodiolus platifrons methanotrophic gill symbiont
Bathymodiolus symbiont pMMOA-1
Batillaria attramentaria
Bdellovibrio bacteriovorus
Bdellovibrio reynosensis
Bdellovibrio sp. KM01
Bdellovibrio sp. NC01
Bdellovibrio sp. SKB1291214
Bdellovibrio sp. ZAP7
Beggiatoa leptomitoformis
Beijerinckia indica subsp. indica ATCC 9039
Beijerinckiaceae bacterium
Bellilinea caldifistulae
Betaproteobacteria bacterium HGW-Betaproteobacteria-11
Betaproteobacteria bacterium HGW-Betaproteobacteria-13
Betaproteobacteria bacterium HGW-Betaproteobacteria-19
Betaproteobacteria bacterium HGW-Betaproteobacteria-1
Betaproteobacteria bacterium
Bifidobacterium animalis subsp. lactis
Biomaibacter acetigenes
Blastococcus sp. TF02-9
Botryobasidium botryosum FD-172 SS1
Brachymonas petroleovorans
Brachyspira aalborgi
Brachyspira alvinipulli
Brachyspira catarrhinii
Brachyspira hampsonii
Brachyspira hyodysenteriae
Brachyspira innocens
Brachyspira intermedia
Brachyspira murdochii
Brachyspira pilosicoli
Brachyspira sp. G79
Brachyspira sp. SAP_772
Brachyspira suanatina
Brachyspira
Bradyrhizobiaceae bacterium
Bradyrhizobium aeschynomenes
Bradyrhizobium brasilense
Bradyrhizobium cajani
Bradyrhizobium diazoefficiens USDA 110
Bradyrhizobium diazoefficiens
Bradyrhizobium diversitatis
Bradyrhizobium forestalis
Bradyrhizobium frederickii
Bradyrhizobium genosp. L
Bradyrhizobium icense
Bradyrhizobium ivorense
Bradyrhizobium japonicum
Bradyrhizobium macuxiense
Bradyrhizobium manausense
Bradyrhizobium neotropicale
Bradyrhizobium niftali
Bradyrhizobium oligotrophicum
Bradyrhizobium ottawaense
Bradyrhizobium rifense
Bradyrhizobium sacchari
Bradyrhizobium shewense
Bradyrhizobium sp. 14AB
Bradyrhizobium sp. 6(2017)
Bradyrhizobium sp. A19
Bradyrhizobium sp. AC87j1
Bradyrhizobium sp. Ai1a-2
Bradyrhizobium sp. CCBAU 45389
Bradyrhizobium sp. CCBAU 51765
Bradyrhizobium sp. CCBAU 53340
Bradyrhizobium sp. CCBAU 53421
Bradyrhizobium sp. CCGB12
Bradyrhizobium sp. CIR18
Bradyrhizobium sp. CIR48
Bradyrhizobium sp. ERR14
Bradyrhizobium sp. I71
Bradyrhizobium sp. IAR9
Bradyrhizobium sp. LCT2
Bradyrhizobium sp. Leo121
Bradyrhizobium sp. Leo170
Bradyrhizobium sp. MOS001
Bradyrhizobium sp. NAS96.2
Bradyrhizobium sp. Oc8
Bradyrhizobium sp. RD5-C2
Bradyrhizobium sp. SEMIA
Bradyrhizobium sp. SSBR45G
Bradyrhizobium sp. SSBR45R
Bradyrhizobium sp. WD16
Bradyrhizobium sp. WSM2254
Bradyrhizobium sp. WSM3983
Bradyrhizobium sp. cir1
Bradyrhizobium sp.
Bradyrhizobium uaiense
Bradyrhizobium zhanjiangense
Bradyrhizobium
Breznakibacter xylanolyticus
Breznakiella homolactica
Brucepastera parasyntrophica
Burkholderia aenigmatica
Burkholderia ambifaria AMMD
Burkholderia ambifaria MC40-6
Burkholderia ambifaria
Burkholderia anthina
Burkholderia arboris
Burkholderia cepacia JBK9
Burkholderia cepacia complex
Burkholderia cepacia
Burkholderia contaminans
Burkholderia glumae LMG 2196 = ATCC 33617
Burkholderia glumae
Burkholderia lata
Burkholderia paludis
Burkholderia plantarii
Burkholderia pyrrocinia
Burkholderia sp. Ac-20345
Burkholderia sp. Ac-20349
Burkholderia sp. Ac-20384
Burkholderia sp. Ac-20392
Burkholderia sp. AgPiMm2-7
Burkholderia sp. BT03
Burkholderia sp. Bp9015
Burkholderia sp. D-99
Burkholderia sp. FL-7-2-10-S1-D7
Burkholderia sp. GbtcB21
Burkholderia sp. H160
Burkholderia sp. IDO3
Burkholderia sp. JKS000303
Burkholderia sp. JP2-270
Burkholderia sp. KBS0801
Burkholderia sp. LMG 13014
Burkholderia sp. MSHR3999
Burkholderia sp. PaNmSr1-4
Burkholderia sp. SJ98
Burkholderia sp. SRS-25
Burkholderia sp. Se-20378
Burkholderia sp. Z1
Burkholderia sp. lig30
Burkholderia stabilis
Burkholderia ubonensis MSMB22
Burkholderia vietnamiensis LMG 10929
Burkholderia vietnamiensis
Burkholderia
Burkholderiales bacterium PBB1
Burkholderiales bacterium RIFCSPHIGHO2_12_63_9
Caballeronia glebae
Caballeronia jiangsuensis
Caballeronia mineralivorans PML1(12)
Caballeronia novacaledonica
Caballeronia sordidicola
Caballeronia temeraria
Caballeronia udeis
Caballeronia zhejiangensis
Caenispirillum bisanense
Caenispirillum salinarum
Caldanaerovirga acetigignens
Caldicellulosiruptor acetigenus
Caldicellulosiruptor bescii
Caldicellulosiruptor danielii
Caldicellulosiruptor diazotrophicus
Caldicellulosiruptor kronotskyensis
Caldicellulosiruptor morganii
Caldicellulosiruptor naganoensis
Caldicellulosiruptor obsidiansis
Caldicellulosiruptor owensensis
Caldicellulosiruptor saccharolyticus
Caldicellulosiruptor
Caldicoprobacter algeriensis
Caldicoprobacter faecalis
Caldicoprobacter guelmensis
Caldinitratiruptor microaerophilus
Calditerrivibrio nitroreducens
Caloramator australicus
Caloramator fervidus
Caloramator proteoclasticus
Caloramator quimbayensis
Caloranaerobacter azorensis
Caloranaerobacter ferrireducens
Caloranaerobacter sp. TR13
Calorimonas adulescens
Caminicella sporogenes
Campylobacter armoricus
Campylobacter coli
Campylobacter fetus
Campylobacter helveticus
Campylobacter hyointestinalis
Campylobacter iguaniorum
Campylobacter insulaenigrae
Campylobacter jejuni
Campylobacter lari
Campylobacter ornithocola
Campylobacter peloridis
Campylobacter sp. 114
Campylobacter sp. 2014D-0216
Campylobacter sp. 46386-21
Campylobacter sp. CNRCH_2013_0855
Campylobacter sp. CNRCH_2014_0184 h
Campylobacter sp. CNRCH_2015_0338 h
Campylobacter sp. CNRCH_2015_0814
Campylobacter sp. CNRCH_2016_0050 h
Campylobacter sp. FU_520
Campylobacter sp. IFREMER_LSEM_CL1890
Campylobacter sp. IFREMER_LSEM_CL2101
Campylobacter sp. IFREMER_LSEM_CL2127
Campylobacter sp. IFREMER_LSEM_CL2151
Campylobacter sp. IFREMER_LSEM_CL2256
Campylobacter sp. P255
Campylobacter sp. RKI_CA19_01121
Campylobacter sp. RM11259
Campylobacter sp. RM13119
Campylobacter sp. RM16704
Campylobacter sputorum
Campylobacter subantarcticus
Campylobacter upsaliensis
Campylobacter volucris
Campylobacter
Candida albicans
Candidatus Accumulibacter aalborgensis
Candidatus Accumulibacter cognatus
Candidatus Accumulibacter contiguus
Candidatus Accumulibacter phosphatis
Candidatus Accumulibacter regalis
Candidatus Accumulibacter sp.
Candidatus Accumulibacter vicinus
Candidatus Brocadia sapporoensis
Candidatus Brocadia sinica
Candidatus Colwellia aromaticivorans
Candidatus Competibacter denitrificans
Candidatus Competibacter phosphatis
Candidatus Contendobacter odensis
Candidatus Dactylopiibacterium carminicum
Candidatus Dormibacteraeota bacterium
Candidatus Endoriftia persephone
Candidatus Formimonas warabiya
Candidatus Frankia alpina
Candidatus Frankia californiensis
Candidatus Koribacter versatilis
Candidatus Kryptonium thompsoni
Candidatus Kuenenia stuttgartiensis
Candidatus Macondimonas diazotrophica
Candidatus Magnetaquicoccus inordinatus
Candidatus Magnetobacterium casensis
Candidatus Marinarcus aquaticus
Candidatus Marithrix sp. Canyon 246
Candidatus Methylacidithermus pantelleriae
Candidatus Methylobacter favarea
Candidatus Methylobacter oryzae
Candidatus Methylomirabilis lanthanidiphila
Candidatus Methylomirabilis limnetica
Candidatus Methylomirabilis oxyfera
Candidatus Methylomirabilis sinica
Candidatus Methylospira mobilis
Candidatus Methylumidiphilus alinenensis
Candidatus Mycobacterium methanotrophicum
Candidatus Mycolicibacterium alkanivorans
Candidatus Nitrosacidococcus sp. I8
Candidatus Nitrosacidococcus tergens
Candidatus Nitrosarchaeum limnium
Candidatus Nitrosocaldus
Candidatus Nitrosocosmicus arcticus
Candidatus Nitrosocosmicus franklandus
Candidatus Nitrosocosmicus hydrocola
Candidatus Nitrosocosmicus oleophilus
Candidatus Nitrosocosmicus sp. SS
Candidatus Nitrosocosmicus
Candidatus Nitrosoglobus terrae
Candidatus Nitrosopelagicus brevis
Candidatus Nitrosopumilus salaria
Candidatus Nitrosopumilus sediminis
Candidatus Nitrosopumilus sp. SW
Candidatus Nitrososphaera evergladensis
Candidatus Nitrososphaera gargensis
Candidatus Nitrosotalea bavarica
Candidatus Nitrosotalea okcheonensis
Candidatus Nitrosotalea sinensis
Candidatus Nitrosotalea sp. FS
Candidatus Nitrosotalea sp. TS
Candidatus Nitrosotalea
Candidatus Nitrosotenuis aquarius
Candidatus Nitrosotenuis cloacae
Candidatus Nitrosotenuis uzonensis
Candidatus Nitrosotenuis
Candidatus Nitrospira inopinata
Candidatus Nitrospira kreftii
Candidatus Nitrospira nitrificans
Candidatus Nitrospira nitrosa
Candidatus Propionivibrio aalborgensis
Candidatus Reidiella endopervernicosa
Candidatus Rhodoblastus alkanivorans
Candidatus Rokubacteria bacterium
Candidatus Scalindua japonica
Candidatus Sulfurimonas baltica
Candidatus Sulfurimonas marisnigri
Candidatus Symbiobacter mobilis
Candidatus Syntrophocurvum alkaliphilum
Candidatus Terasakiella magnetica
Candidatus Thiodiazotropha endoloripes
Candidatus Thiodiazotropha endolucinida
Capillibacterium thermochitinicola
Capnocytophaga canis
Caproicibacterium amylolyticum
Caproiciproducens sp. AGMB10547
Carbonactinospora thermoautotrophica
Carboxydocella
Carboxylicivirga linearis
Carboxylicivirga marina
Carboxylicivirga mesophila
Carboxylicivirga sediminis
Carboxylicivirga sp. A043
Carboxylicivirga sp. M1479
Catellatospora citrea
Caulobacter sp. BK020
Cecembia calidifontis
Cecembia rubra
Celeribacter baekdonensis B30
Celeribacter baekdonensis
Cellulomonas sp. SLBN-39
Cellulosilyticum sp. I15G10I2
Cereibacter johrii
Cereibacter sphaeroides 2.4.1
Cereibacter sphaeroides KD131
Cereibacter sphaeroides WS8N
Cereibacter sphaeroides f. sp. denitrificans
Cereibacter sphaeroides
Chelativorans sp. Marseille-P2723
Chelativorans
Chitiniphilus shinanonensis
Chitinivibrio alkaliphilus
Chitinophaga dinghuensis
Chitinophaga ginsengisegetis
Chitinophaga ginsengisoli
Chitinophaga japonensis
Chitinophaga niastensis
Chitinophaga polysaccharea
Chitinophaga sp. S165
Chloroflexi bacterium ADurb.Bin120
Chloroflexi bacterium ADurb.Bin180
Chloroflexota bacterium
Chloroherpeton thalassium
Christensenella intestinihominis
Chromatiales bacterium USCg_Taylor
Chrysiogenes arsenatis
Chthoniobacter flavus
Ciceribacter selenitireducens ATCC BAA-1503
Citreicella sp. SE45
Citrifermentans bemidjiense
Citrifermentans bremense
Citrifermentans pelophilum
Citrifermentans
Clostridiisalibacter paucivorans
Clostridioides difficile
Clostridium aceticum
Clostridium acetireducens
Clostridium acetobutylicum
Clostridium aciditolerans
Clostridium aestuarii
Clostridium algidicarnis
Clostridium algifaecis
Clostridium amylolyticum
Clostridium argentinense
Clostridium autoethanogenum
Clostridium beijerinckii
Clostridium botulinum
Clostridium caldaquaticum
Clostridium carboxidivorans
Clostridium carnis
Clostridium cellulovorans
Clostridium chromiireducens
Clostridium fallax
Clostridium formicaceticum
Clostridium frigidicarnis
Clostridium frigoris
Clostridium fungisolvens
Clostridium ganghwense
Clostridium gelidum
Clostridium homopropionicum
Clostridium hydrogeniformans
Clostridium indicum
Clostridium intestinale
Clostridium ljungdahlii
Clostridium magnum
Clostridium manihotivorum
Clostridium muellerianum
Clostridium neonatale
Clostridium paridis
Clostridium peptidivorans
Clostridium polynesiense
Clostridium punense
Clostridium puniceum
Clostridium putrefaciens
Clostridium rectalis
Clostridium rhizosphaerae
Clostridium saccharobutylicum
Clostridium saccharoperbutylacetonicum
Clostridium sartagoforme
Clostridium sp. ‘White wine YQ’
Clostridium sp. ‘deep sea’
Clostridium sp. 1001275B_160808_H3
Clostridium sp. AF34-10BH
Clostridium sp. AM34-9AC
Clostridium sp. AWRP
Clostridium sp. BL-8
Clostridium sp. BNL1100
Clostridium sp. C2-6-12
Clostridium sp. C8-1-8
Clostridium sp. C8
Clostridium sp. CX1
Clostridium sp. Cult1
Clostridium sp. Cult2
Clostridium sp. Cult3
Clostridium sp. D1-1
Clostridium sp. DJ247
Clostridium sp. DL-VIII
Clostridium sp. E02
Clostridium sp. E14
Clostridium sp. HBUAS56010
Clostridium sp. JN-1
Clostridium sp. JN-9
Clostridium sp. KNHs216
Clostridium sp. LS
Clostridium sp. Marseille-P2415
Clostridium sp. Marseille-P299
Clostridium sp. Marseille-P3244
Clostridium sp. Marseille-Q2269
Clostridium sp. N3C
Clostridium sp. YIM B02506
Clostridium sp. YIM B02551
Clostridium sp. YIM B02555
Clostridium sp. ZC22-4
Clostridium swellfunianum
Clostridium thailandense
Clostridium weizhouense
Clostridium yunnanense
Clostridium zeae
Clostridium
Cognatazoarcus halotolerans
Cognaticolwellia aestuarii
Cognaticolwellia mytili
Colidextribacter sp. 210702-DFI.3.9
Collinsella bouchesdurhonensis
Collinsella sp. KGMB02528
Colwellia chukchiensis
Colwellia demingiae
Colwellia echini
Colwellia psychrerythraea
Colwellia sp. 12G3
Colwellia sp. 20A7
Colwellia sp. 75C3
Colwellia sp. Arc7-635
Colwellia sp. BRX8-9
Colwellia sp. Bg11-28
Colwellia sp. D2M02
Colwellia sp. E2M01
Colwellia sp. M166
Colwellia sp. MSW7
Colwellia sp. RSH04
Colwellia sp.
Colwellia
Comamonadaceae bacterium
Comamonas denitrificans
Comamonas granuli
Comamonas sp. CMM03
Comamonas terrigena
Comamonas testosteroni
Comamonas
Conexibacter sp. DBS9H8
Conexibacter sp. S30A1
Conexibacter sp. SYSU D00693
Conexibacter sp. W3-3-2
Conexibacter woesei
Congregibacter sp.
Coprothermobacter platensis
Coprothermobacter proteolyticus
Crassaminicella profunda
Crassaminicella sp. 143-21
Crassaminicella thermophila
Craterilacuibacter sp. RT1T
Crenothrix polyspora
Crenothrix sp. D3
Cryobacterium sp. SO1
Cryptosporangium arvum
Cryptosporangium aurantiacum
Cryptosporangium phraense
Cuneatibacter sp. NSJ-177
Cupidesulfovibrio liaohensis
Cupidesulfovibrio oxamicus
Cupidesulfovibrio sp. HK-II
Cupidesulfovibrio sp. SRB-5
Cupidesulfovibrio termitidis
Cupriavidus lacunae
Cupriavidus necator N-1
Cupriavidus necator
Cupriavidus pinatubonensis
Cupriavidus sp. IDO
Curtobacterium citreum
Curtobacterium sp. AG1037
Curtobacterium sp. JUb34
Curvibacter delicatus
Curvibacter gracilis
Curvibacter lanceolatus
Curvibacter sp. PAE-UM
Curvivirga aplysinae
Cuspidothrix issatschenkoi
Cyanobacterium aponinum
Cyanobacterium sp. IPPAS B-1200
Cyanobacterium stanieri
Dechloromonas agitata
Dechloromonas denitrificans
Dechloromonas hortensis
Dechloromonas sp. A34
Dechloromonas sp. HYN0024
Dechloromonas sp. TW-R-39-2
Deferribacter autotrophicus
Deferribacter desulfuricans
Deferrisoma camini
Deferrivibrio essentukiensis
Defluviicoccus vanus
Defluviimonas aquaemixtae
Defluviitalea phaphyphila
Defluviitalea raffinosedens
Deinococcus saudiensis
Deltaproteobacteria bacterium
Dendrosporobacter quercicolus
Denitratisoma oestradiolicum
Denitratisoma sp. DHT3
Denitromonas halophilus
Denitromonas sp. IR12
Denitrovibrio acetiphilus
Derxia gummosa
Derxia lacustris
Desnuesiella massiliensis
Desulfacinum hydrothermale
Desulfacinum infernum
Desulfamplus magnetovallimortis
Desulfatibacillum aliphaticivorans
Desulfatibacillum alkenivorans
Desulfitibacter alkalitolerans
Desulfobacter curvatus
Desulfobacter latus
Desulfobacter postgatei
Desulfobacter vibrioformis
Desulfobacula phenolica
Desulfobacula toluolica
Desulfobaculum xiamenense
Desulfobotulus alkaliphilus
Desulfobotulus mexicanus
Desulfobotulus sp. H1
Desulfobulbus elongatus
Desulfobulbus propionicus
Desulfobulbus rhabdoformis
Desulfocapsa sulfexigens
Desulfocicer vacuolatum
Desulfocurvibacter africanus
Desulfocurvus vexinensis
Desulfofalx alkaliphila
Desulfoglaeba alkanexedens
Desulfogranum japonicum
Desulfogranum mediterraneum
Desulfohalovibrio alkalitolerans
Desulfohalovibrio reitneri
Desulfolithobacter dissulfuricans
Desulfoluna butyratoxydans
Desulfoluna sp. ASN36
Desulfoluna spongiiphila
Desulfolutivibrio sulfoxidireducens
Desulfomarina profundi
Desulfomicrobium apsheronum
Desulfomicrobium baculatum
Desulfomicrobium escambiense
Desulfomicrobium macestii
Desulfomicrobium norvegicum
Desulfomicrobium sp. ZS1
Desulfomonile tiedjei
Desulfonatronum lacustre
Desulfonatronum thioautotrophicum
Desulfonatronum thiodismutans
Desulfonema ishimotonii
Desulfonema limicola
Desulfonema magnum
Desulfonispora thiosulfatigenes
Desulfopila aestuarii
Desulfopila sp. IMCC35006
Desulfopila sp. IMCC35008
Desulfoplanes formicivorans
Desulfoprunum benzoelyticum
Desulforapulum autotrophicum
Desulforegula conservatrix
Desulforhopalus singaporensis
Desulforhopalus sp. IMCC35007
Desulfosediminicola flagellatus
Desulfosoma caldarium
Desulfospira joergensenii
Desulfotignum balticum
Desulfotignum phosphitoxidans
Desulfovibrio aminophilus
Desulfovibrio desulfuricans
Desulfovibrio fairfieldensis
Desulfovibrio ferrophilus
De sulfovibrio gilichinskyi
Desulfovibrio inopinatus
Desulfovibrio intestinalis
Desulfovibrio legallii
Desulfovibrio litoralis
Desulfovibrio oxyclinae
Desulfovibrio porci
Desulfovibrio psychrotolerans
Desulfovibrio sp. 3_1_syn3
Desulfovibrio sp. 6_1_46AFAA
Desulfovibrio sp. 86
Desulfovibrio sp. A2
Desulfovibrio sp. DV
Desulfovibrio sp. Fe33
Desulfovibrio sp. JC010
Desulfovibrio sp. JC022
Desulfovibrio sp. TomC
Desulfovibrio sp. UIB00
Desulfovibrio sp. X2
Desulfovibrio sp. ZJ200
Desulfovibrio sp. ZJ369
Desulfovibrio subterraneus
Desulfovibrio sulfodismutans
Desulfovibrio vulgaris
Desulfovibrio
Desulfuribacillus alkaliarsenatis
Desulfuribacillus stibiiarsenatis
Desulfurispira natronophila
Desulfurispirillum indicum
Desulfurivibrio alkaliphilus
Desulfuromonas acetexigens
Desulfuromonas acetoxidans
Desulfuromonas soudanensis
Desulfuromonas sp. AOP6
Desulfuromonas sp. CSMB_57
Desulfuromonas sp. DDH964
Desulfuromonas sp. KJ2020
Desulfuromonas sp. TF
Desulfuromonas thiophila
Desulfuromonas versatilis
Desulfuromusa kysingii
Dethiosulfatarculus sandiegensis
Diaminobutyricimonas aerilata
Diaphorobacter sp. ED-3
Diaphorobacter sp. LR2014-1
Dietzia sp. ANT_WB102
Dioscorea alata
Dissulfurimicrobium hydrothermale
Dissulfurispira thermophila
Dongshaea marina
Dorea amylophila
Dorea longicatena
Dyadobacter jejuensis
Dyadobacter jiangsuensis
Dyella sp. NS14
Dyella sp. Sa
Edaphobacter aggregans
Edaphobacter modestus
Endozoicomonas ascidiicola
Endozoicomonas atrinae
Enemella dayhoffiae
Enemella evansiae
Ensifer sp. LCM 4579
Enterocloster bolteae
Enterococcus faecalis 02-MB-P-10
Enterovibrio norvegicus
Enterovibrio pacificus
Enterovibrio paralichthyis
Enterovibrio sp. ZSDZ42
Epulopiscium sp. ‘N.t. morphotype B’
Ethanoligenens harbinense
Eubacteriales
Eubacterium ramulus
Eubacterium sp. 14-2
Eubacterium sp. MSJ-13
Euhalothece natronophila
Faecalimonas umbilicata
Falcatimonas sp. MSJ-15
Ferribacterium limneticum
Ferriphaselus amnicola
Ferrithrix thermotolerans
Ferrovum myxofaciens
Ferrovum sp. PN-J185
Fervidicola ferrireducens
Fibrisoma limi BUZ 3
Flavobacteriaceae bacterium MAR_2009_75
Flavonifractor sp. An306
Flexistipes sinusarabici
Flexivirga oryzae
Fluviicoccus keumensis
Fodinicola acaciae
Formivibrio citricus
Francisella sp. SYW-9
Frankia casuarinae
Frankia discariae
Frankia sp. B2
Frankia sp. CcI156
Frankia sp. CgIM4
Frankia sp. CgIS1
Frankia sp. EUN1f
Frankia sp. KB5
Frankia sp. R43
Frankia
Frigoribacterium sp. Leaf44
Fuchsiella alkaliacetigena
Fulvimonas soli
Fundidesulfovibrio magnetotacticus
Fundidesulfovibrio putealis
Fundidesulfovibrio soli
Fundidesulfovibrio sp. SG106
Fundidesulfovibrio sp. SG127
Fusibacter ferrireducens
Fusibacter paucivorans
Fusibacter sp. 3D3
Fusibacter tunisiensis
Gallionella capsiferriformans
Gammaproteobacteria bacterium BFH2
Gammaproteobacteria bacterium MOLA455
Gammaproteobacteria bacterium
Geminocystis herdmanii
Geminocystis sp. NIES-3708
Gemmatimonas groenlandica
Gemmobacter nanjingensis
Geoalkalibacter ferrihydriticus
Geoalkalibacter halelectricus
Geoalkalibacter subterraneus
Geobacter anodireducens
Geobacter argillaceus
Geobacter benzoatilyticus
Geobacter chapellei
Geobacter grbiciae
Geobacter hydrogenophilus
Geobacter luticola
Geobacter metallireducens
Geobacter pickeringii
Geobacter soli
Geobacter sp. AOG1
Geobacter sp. AOG2
Geobacter sp. DSM 9736
Geobacter sp. FeAm09
Geobacter sp. OR-1
Geobacter sp. SVR
Geobacter sulfurreducens
Geobacter
Geodermatophilus dagingensis
Geodermatophilus ruber
Geofilum rhodophaeum
Geofilum rubicundum
Geomesophilobacter sediminis
Geomonas agri
Geomonas anaerohicana
Geomonas azotofigens
Geomonas diazotrophica
Geomonas edaphica
Geomonas ferrireducens
Geomonas fuzhouensis
Geomonas limicola
Geomonas nitrogeniifigens
Geomonas oryzae
Geomonas oryzisoli
Geomonas paludis
Geomonas propionica
Geomonas silvestris
Geomonas sp. RF6
Geomonas sp. Red32
Geomonas subterranea
Geomonas terrae
Geomonas
Geopsychrobacter electrodiphilus
Georgfuchsia toluolica
Geosporobacter ferrireducens
Geosporobacter subterraneus
Geotalea daltonii
Geotalea sp. SG265
Geotalea toluenoxydans
Geotalea uraniireducens
Geothermobacter ehrlichii
Geothermobacter hydrogeniphilus
Geothrix fermentans
Geothrix sp. SG10
Geothrix sp. SG12
Geothrix sp. SG164
Geothrix sp. SG178
Geothrix sp. SG184
Geothrix sp. SG263
Geovibrio ferrireducens
Geovibrio thiophilus
Giesbergeria anulus
Glycomyces artemisiae
Gordonia alkanivorans CGMCC 6845
Gordonia alkanivorans
Gordonia amarae
Gordonia asplenii
Gordonia bronchialis DSM 43247
Gordonia bronchialis
Gordonia humi
Gordonia hydrophobica
Gordonia insulae
Gordonia jacobaea
Gordonia jinghuaigii
Gordonia lacunae
Gordonia liuliyuniae
Gordonia mangrovi
Gordonia oryzae
Gordonia polyisoprenivorans
Gordonia rhizosphera
Gordonia rubripertincta
Gordonia sp. ‘Campus'
Gordonia sp. 135
Gordonia sp. 1D
Gordonia sp. 852002-10350_SCH5691597
Gordonia sp. 852002-50816_SCH5313054-a
Gordonia sp. 852002-50816_SCH5313054-c
Gordonia sp. ABSL49_1
Gordonia sp. CNJ-863
Gordonia sp. GONU
Gordonia sp. KTR9
Gordonia sp. NB41Y
Gordonia sp. OPL2
Gordonia sp. PDNC005
Gordonia sp. SW 21
Gordonia sp. i37
Gordonia spumicola
Gordonia tangerina
Gordonia terrae C-6
Gordonia terrae
Gordonia westfalica
Gordonia zhenghanii
Gordonia
Gorillibacterium massiliense
Gorillibacterium timonense
Gossypium arboreum
Gottschalkia purinilytica
Gracilinema caldarium
Granulicella mallensis
Granulicoccus phenolivorans
Granulosicoccus antarcticus IMCC3135
Haematobacter missouriensis
Hahella ganghwensis
Hahella sp. CCB-MM4
Halalkalibacterium ligniniphilum
Halanaerobacter jeridensis
Halanaerobium praevalens
Halanaerobium saccharolyticum
Halanaerobium salsuginis
Halarcobacter anaerophilus
Halarcobacter bivalviorum
Halarcobacter mediterraneus
Haliea sp. ETY-M
Haliea sp. ETY-NAG
Haliea sp. SAOS-164
Haloactinopolyspora alba
Haloarcula amylolytica
Haloarcula argentinensis
Haloarcula californiae
Haloarcula hispanica
Haloarcula japonica
Haloarcula mannanilytica
Haloarcula marismortui
Haloarcula rubripromontorii
Haloarcula sebkhae
Haloarcula sinaiiensis
Haloarcula sp. Atlit-7R
Haloarcula sp. CBA1122
Haloarcula sp. CBA1127
Haloarcula sp. JP-Z28
Haloarcula vallismortis
Haloarcula
Halobacteriovorax marinus
Halobacteriovorax sp. BALOs_7
Halobacteriovorax sp. DA5
Halobacteriovorax sp. GB3
Halobacteriovorax sp. HLS
Halobacteriovorax
Halobacteroides halobius
Halobellus inordinatus
Halobellus litoreus
Halobellus rarus
Halobellus sp. Atlit-38R
Halobiforma lacisalsi
Halochromatium glycolicum
Halochromatium roseum
Halochromatium salexigens
Halodesulfovibrio aestuarii
Halodesulfovibrio marinisediminis
Halodesulfovibrio sp. MK-HDV
Halodesulfovibrio spirochaetisodalis
Haloechinothrix alba
Haloechinothrix halophila
Haloglomus sp. ZY41
Haloimpatiens massiliensis
Halomicroarcula salinisoli
Halomicrobium katesii
Halomicrobium mukohataei
Halomicrobium sp. IBSBa
Halomicrobium
Halonatronum saccharophilum
Halopolyspora algeriensis
Halorientalis brevis
Halorientalis litorea
Halorientalis marina
Halorientalis pallida
Halorientalis persicus
Halorientalis salina
Halosaccharopolyspora lacisalsi
Halosimplex pelagicum
Halosimplex rubrum
Halothece sp. PCC 7418
Halothermothrix orenii
Halovenus aranensis
Halovenus salina
Hartmannibacter diazotrophicus
Hathewaya histolytica
Hathewaya massiliensis
Hathewaya proteolytica
Helicobacter apodemus
Helicobacter burdigaliensis
Helicobacter canis
Helicobacter himalayensis
Helicobacter mesocricetorum
Helicobacter pullorum
Helicobacter sp. 15-1451
Helicobacter sp. MIT 05-5293
Helicobacter sp. MIT 99-5507
Heliobacterium chlorum
Heliobacterium mobile
Heliomicrobium gestii
Heliomicrobium modesticaldum
Heliomicrobium undosum
Heliorestis acidaminivorans
Heliorestis convoluta
Herbaspirillum sp. ST 5-3
Herbihabitans rhizosphaerae
Holophaga foetida
Hoyosella altamirensis
Hoyosella subflava
Humibacillus sp. DSM 29435
Humibacillus xanthopallidus
Humidesulfovibrio mexicanus
Hungatella effluvii
Hungatella xylanolytica
Hwanghaeella grinnelliae
Hydrocarboniphaga effusa
Hydrogenibacillus schlegelii
Hydrogenispora ethanolica
Hydrogenoanaerobacterium saccharovorans
Hydrogenobacter sp. T-8
Hydrogenophaga aromaticivorans
Hydrogenophaga crassostreae
Hydrogenophaga pseudoflava
Hydrogenophaga sp. PAMC20947
Hydrogenophaga sp. T4
Hydrogenophaga taeniospiralis
Hydrogenophilus thermoluteolus
Hydrogenophilus thiooxidans
Hyphomicrobium sp. CS1BSMeth3
Hyphomicrobium sp.
Ideonella sp. A 288
Idiomarina abyssalis
Idiomarina piscisalsi
Idiomarina seosinensis
Idiomarina sp. 29L
Idiomarina sp. M1R2S28
Idiomarina sp. X4
Idiomarina sp.
Idiomarina zobellii
Idiomarina
Ignavibacteria bacterium ADurb.Bin266
Imhoffiella purpurea
Inediibacterium massiliense
Inhella gelatinilytica
Inoviridae sp.
Inquilinus limosus
Insolitispirillum peregrinum
Intestinibacillus sp. Marseille-P6563
Intestinimonas sp. MSJ-38
Intrasporangium oryzae NRRL B-24470
Intrasporangium oryzae
Iocasia fonsfrigidae
Iodobacter ciconiae
Isoptericola halotolerans
Isoptericola sp. CG 20/1183
Isoptericola variabilis J7
Isosphaera pallida ATCC 43644
Janthinobacterium sp. 17J80-10
Jatrophihabitans endophyticus
Kaistella jeonii
Keratinibaculum paraultunense
Kibdelosporangium aridum
Kibdelosporangium banguiense
Kibdelosporangium philippinense
Kineococcus rhizosphaerae
Kineococcus xinjiangensis
Kineosporia babensis
Kineosporia rhizophila
Kineosporia sp. A_224
Kineosporia sp. R_H_3
Kistimonas asteriae
Knoellia sp. DB2414S
Kordiimonas marina
Kribbella aluminosa
Kribbella amoyensis
Kribbella antibiotica
Kribbella capetownensis
Kribbella italica
Kribbella jejuensis
Kribbella jiaozuonensis
Kribbella monticola
Kribbella pittospori
Kribbella gitaiheensis
Kribbella shirazensis
Kribbella sindirgiensis
Kribbella soli
Kribbella sp. VKM Ac-2500
Kribbella sp. VKM Ac-2527
Kribbella sp. VKM Ac-2538
Kribbella sp. VKM Ac-2541
Kribbella sp. VKM Ac-2566
Kribbella sp. VKM Ac-2568
Kribbella sp. VKM Ac-2569
Kribbella sp. VKM Ac-2570
Kribbella sp. VKM Ac-2571
Kribbella sp. VKM Ac-2572
Kribbella sp. VKM Ac-2573
Kribbella sp. VKM Ac-2575
Kribbella sp. VKM Ac-2574
Kribbella speibonae
Kribbella
Ktedonobacter sp. 13_1_20CM_3_54_15
Ktedonobacter sp. 13_2_20CM_53_11
Kutzneria buriramensis
Kutzneria chonburiensis
Kutzneria kofuensis
Kutzneria sp. 744
Kutzneria sp. CA-103260
Kyrpidia spormannii
Kyrpidia tusciae
Labedaea rhizosphaerae
Labilibacter marinus
Labilithrix luteola
Labrys miyagiensis
Labrys okinawensis
Labrys sp. KNU-23
Labrys sp. WJW
Lachnobacterium bovis
Lachnoclostridium sp. An138
Lachnoclostridium sp. An76
Lachnospira eligens
Lachnospira pectinoschiza
Lachnospiraceae
Lacrimispora aerotolerans
Lacrimispora algidixylanolytica
Lacrimispora amygdalina
Lacrimispora celerecrescens
Lacrimispora saccharolytica
Lacrimispora sphenoides
Lacrimispora xylanolytica
Lacrimispora
Lactonifactor longoviformis
Lactonifactor
Lamprobacter modestohalophilus
Larkinella arboricola
Leadbettera azotonutricia
Leekyejoonella antrihumi
Legionella massiliensis
Leifsonia shinshuensis
Leifsonia sp. C5G2
Leisingera daeponensis
Leisingera sp. ANG59
Leisingera
Lentibacillus sp. ZS110521
Lentilactobacillus buchneri DSM 20057
Lentilactobacillus buchneri
Lentilactobacillus parabuchneri DSM 5707 = NBRC 107865
Lentilactobacillus parabuchneri
Lentzea albidocapillata
Lentzea atacamensis
Lentzea flaviverrucosa
Leptolinea tardivitalis
Leptolyngbya sp. BL0902
Leptolyngbya sp. CCY15150
Leptospira abararensis
Leptospira bandrabouensis
Leptospira biflexa
Leptospira bourretii
Leptospira bouyouniensis
Leptospira brenneri
Leptospira chreensis
Leptospira congkakensis
Leptospira ellinghausenii
Leptospira harrisiae
Leptospira idonii
Leptospira jelokensis
Leptospira kanakyensis
Leptospira kemamanensis
Leptospira kobayashii
Leptospira levettii
Leptospira meyeri
Leptospira mtsangambouensis
Leptospira noumeaensis
Leptospira ognonensis
Leptospira perdikensis
Leptospira sp. 1 VSF14
Leptospira sp. 3 VSF25
Leptospira sp. GIMC2001
Leptospira sp. mixed culture ATI2-C-A1
Leptospira terpstrae
Leptospira vanthielii
Leptospira wolbachii
Leptospira yanagawae
Leptospira
Lichenicoccus roseus
Limihaloglobus sulfuriphilus
Limisalsivibrio acetivorans
Litorilituus lipolyticus
Litorilituus sediminis
Lucifera butyrica
Lutibacter sp. HS1-25
Lutispora thermophila
Luxibacter massiliensis
Magnetococcus marinus
Magnetofaba australis
Magnetospira sp. QH-2
Magnetospirillum aberrantis
Magnetospirillum caucaseum
Magnetospirillum fulvum
Magnetospirillum gryphiswaldense
Magnetospirillum kuznetsovii
Magnetospirillum magneticum
Magnetospirillum magnetotacticum
Magnetospirillum marisnigri
Magnetospirillum molischianum
Magnetospirillum moscoviense
Magnetospirillum sp. 15-1
Magnetospirillum sp. J10
Magnetospirillum sp. LM-5
Magnetospirillum sp. ME-1
Magnetospirillum sp. SS-4
Magnetospirillum sp. UT-4
Magnetospirillum sp. XM-1
Magnetospirillum
Magnetovibrio blakemorei
Mahella australiensis
Malaciobacter mytili
Maledivibacter halophilus
Malonomonas rubra
Mameliella alba
Mangrovibacterium diazotrophicum
Mangrovibacterium marinum
Maribacter polysiphoniae
Maribacter sp. MAR_2009_72
Maribellus comscasis
Maribellus maritimus
Maricaulis alexandrii
Maricaulis parjimensis
Maricaulis virginensis
Maridesulfovibrio bastinii
Maridesulfovibrio ferrireducens
Maridesulfovibrio frigidus
Maridesulfovibrio hydrothermalis
Maridesulfovibrio salexigens
Maridesulfovibrio zosterae
Marinilabilia rubra
Marinilabilia salmonicolor
Marinisporobacter balticus
Marinithermofilum abyssi
Marinobacter antarcticus
Marinobacter aromaticivorans
Marinobacter bryozoorum
Marinobacter dagiaonensis
Marinobacter litoralis
Marinobacter nauticus
Marinobacter pelagius
Marinobacter persicus
Marinobacter profundi
Marinobacter salexigens
Marinobacter sp. 1-4A
Marinobacter sp. DY40_1A1
Marinobacter sp. EVN1
Marinobacter sp. G11
Marinobacter sp. S6332
Marinobacter sp. bablab_jr008
Marinobacter sp.
Marinobacter vinifirmus
Marinobacter zhanjiangensis
Marinobacter zhejiangensis
Marinobacter
Marinobacterium jannaschii
Marinobacterium sp. A346
Marinomonas atlantica
Marinomonas fungiae
Marinomonas primoryensis
Marinovum algicola
Mariprofundus aestuarium
Mariprofundus erugo
Mariprofundus ferrooxydans
Mariprofundus micogutta
Mariprofundus sp. EBB-1
Mariprofundus sp. KV
Mariprofundus sp. NF
Marmoricola pocheonensis
Marortus luteolus
Martelella
Marvinbryantia formatexigens
Mediterraneibacter glycyrrhizinilyticus
Megalodesulfovibrio gigas
Meiothermus sp. CFH 77666
Melaminivora jejuensis
Melioribacter roseus
Mesorhizobium amorphae CCNWGS0123
Mesorhizobium amorphae
Mesorhizobium atlanticum
Mesorhizobium delmotii
Mesorhizobium hawassense
Mesorhizobium helmanticense
Mesorhizobium kowhaii
Mesorhizobium loti
Mesorhizobium mediterraneum
Mesorhizobium onobrychidis
Mesorhizobium prunaredense
Mesorhizobium sp. 113-1-2
Mesorhizobium sp. 113-3-9
Mesorhizobium sp. 65-26
Mesorhizobium sp. B2-1-3A
Mesorhizobium sp. B2-3-11
Mesorhizobium sp. B2-3-3
Mesorhizobium sp. B2-3-5
Mesorhizobium sp. B2-4-12
Mesorhizobium sp. B2-4-14
Mesorhizobium sp. B2-4-15
Mesorhizobium sp. B2-4-17
Mesorhizobium sp. B2-4-19
Mesorhizobium sp. B2-5-13
Mesorhizobium sp. B2-5-5
Mesorhizobium sp. B2-8-3
Mesorhizobium sp. B2-8-9
Mesorhizobium sp. B3-1-3
Mesorhizobium sp. B3-1-6
Mesorhizobium sp. B3-1-7
Mesorhizobium sp. B3-1-8
Mesorhizobium sp. B3-1-9
Mesorhizobium sp. B3-2-1
Mesorhizobium sp. DCY119
Mesorhizobium sp. L-2-11
Mesorhizobium sp. L-8-10
Mesorhizobium sp. L-8-3
Mesorhizobium sp. L48C026A00
Mesorhizobium sp. LNHC252B00
Mesorhizobium sp. LSJC280B00
Mesorhizobium sp. M2A.F.Ca.ET.043.02.1.1
Mesorhizobium sp. M2C.T.Ca.TU.002.02.1.1
Mesorhizobium sp. M2C.T.Ca.TU.009.01.2.1
Mesorhizobium sp. M2E.F.Ca.ET.154.01.1.1
Mesorhizobium sp. M2E.F.Ca.ET.166.01.1.1
Mesorhizobium sp. M2E.F.Ca.ET.219.01.1.1
Mesorhizobium sp. M4A.F.Ca.ET.020.02.1.1
Mesorhizobium sp. M4A.F.Ca.ET.022.05.2.1
Mesorhizobium sp. M4A.F.Ca.ET.029.04.2.1
Mesorhizobium sp. M4A.F.Ca.ET.050.02.1.1
Mesorhizobium sp. M4A.F.Ca.ET.090.04.2.1
Mesorhizobium sp. M4B.F.Ca.ET.058.02.1.1
Mesorhizobium sp. M5C.F.Ca.ET.164.01.1.1
Mesorhizobium sp. M5C.F.Cr.IN.023.01.1.1
Mesorhizobium sp. M6A.T.Ca.TU.002.02.2.1
Mesorhizobium sp. M6A.T.Ce.TU.002.03.1.1
Mesorhizobium sp. M6A.T.Ce.TU.016.01.1.1
Mesorhizobium sp. M6A.T.Cr.TU.014.01.1.1
Mesorhizobium sp. M6A.T.Cr.TU.016.01.1.1
Mesorhizobium sp. M6A.T.Cr.TU.017.01.1.1
Mesorhizobium sp. M7A.F.Ca.US.010.02.1.1
Mesorhizobium sp. M7A.F.Ca.US.011.01.1.1
Mesorhizobium sp. M7D.F.Ca.US.004.01.2.1
Mesorhizobium sp. M7D.F.Ca.US.004.03.1.1
Mesorhizobium sp. M7D.F.Ca.US.005.01.1.1
Mesorhizobium sp. M9A.F.Ca.ET.002.03.1.2
Mesorhizobium sp. NZP2234
Mesorhizobium sp. NZP2298
Mesorhizobium sp. ORS 3428
Mesorhizobium sp. Root172
Mesorhizobium sp. Root552
Mesorhizobium sp. SARCC-RB16n
Mesorhizobium sp. SCN 65-12
Mesorhizobium sp. WSM3860
Mesorhizobium sp. WSM4303
Mesorhizobium sp. WSM4306
Mesorhizobium sp.
Mesorhizobium tamadayense
Mesorhizobium temperatum
Mesorhizobium tianshanense
Mesorhizobium waimense
Mesorhizobium wenxiniae
Metallosphaera javensis (ex Hofmann et al. 2022)
Metallosphaera sedula
Methanobacterium formicicum
Methanocaldococcus infernus
Methanofollis ethanolicus
Methanohalophilus levihalophilus
Methanolacinia paynteri
Methanolacinia petrolearia
Methanolobus bombayensis
Methanolobus profundi
Methanolobus psychrotolerans
Methanolobus tindarius
Methanolobus vulcani
Methanolobus zinderi
Methanomicrobium sp. W14
Methanonatronarchaeum thermophilum
Methanoregula boonei
Methanosarcina horonobensis
Methanosphaerula palustris
Methanospirillum hungatei
Methanospirillum lacunae
Methanospirillum sp. J.3.6.1-F.2.7.3
Methanospirillum stamsii
Methanospirillum
Methylacidimicrobium cyclopophantes
Methylacidimicrobium fagopyrum
Methylacidimicrobium sp. AP8
Methylacidimicrobium sp. B4
Methylacidimicrobium tartarophylax
Methylacidiphilum fumariolicum SolV
Methylacidiphilum fumariolicum
Methylacidiphilum infernorum V4
Methylacidiphilum infernorum
Methylacidiphilum kamchatkense Kam1
Methylacidiphilum kamchatkense
Methylacidiphilum sp. IT5
Methylacidiphilum sp. IT6
Methylacidiphilum sp. Phi
Methylacidiphilum sp. RTK17.1
Methylacidiphilum sp. Yel
Methylacidiphilum
Methylibium sp. Pch-M
Methylibium sp. Root1272
Methylicorpusculum oleiharenae
Methylobacter luteus
Methylobacter marinus
Methylobacter psychrophilus
Methylobacter sp. 5FB
Methylobacter sp. BB5.1
Methylobacter sp. BBA5.1
Methylobacter sp. BlB1
Methylobacter sp. CMS7
Methylobacter sp. LW1
Methylobacter sp. S3L5C
Methylobacter sp. YRD-M1
Methylobacter sp. clone FIN1
Methylobacter sp. clone FIN2
Methylobacter sp. clone FIN3
Methylobacter sp. clone FIN4
Methylobacter sp. clone FIN5
Methylobacter sp. clone PP39C
Methylobacter sp. clone PP39D
Methylobacter sp.
Methylobacter tundripaludum SV96
Methylobacter tundripaludum
Methylobacter
Methylobacterium brachiatum
Methylobacterium bullatum
Methylobacterium dankookense
Methylobacterium fujisawaense
Methylobacterium longum
Methylobacterium oryzae CBMB20
Methylobacterium oryzae
Methylobacterium persicinum
Methylobacterium phyllosphaerae
Methylobacterium radiotolerans
Methylobacterium sp.
Methylobacterium symbioticum
Methylocaldum gracile
Methylocaldum marinum
Methylocaldum sp. 14B
Methylocaldum sp. 5FB
Methylocaldum sp. BFH1
Methylocaldum sp. E10_A
Methylocaldum sp. ML100
Methylocaldum sp. ML184
Methylocaldum sp. ML39
Methylocaldum sp. ML65
Methylocaldum sp. RMAD-M
Methylocaldum sp. T-025
Methylocaldum sp. enrichment culture clone Lpl2-123
Methylocaldum sp. enrichment culture clone Lpl2-128
Methylocaldum sp.
Methylocaldum szegediense
Methylocaldum tepidum
Methylocapsa acidiphila B2
Methylocapsa acidiphila
Methylocapsa aurea
Methylocapsa palsarum
Methyloceanibacter marginalis
Methyloceanibacter methanicus
Methyloceanibacter stevinii
Methyloceanibacter superfactus
Methylocella palustris
Methylocella silvestris BL2
Methylocella silvestris
Methylocella tundrae
Methylococcaceae bacterium AK-K6
Methylococcaceae bacterium BRS-K6
Methylococcaceae bacterium ET-HIRO
Methylococcaceae bacterium ET-SHO
Methylococcaceae bacterium M200
Methylococcaceae bacterium SF-BR
Methylococcaceae bacterium
Methylococcales bacterium
Methylococcus capsulatus str. Bath
Methylococcus capsulatus
Methylococcus geothermalis
Methylococcus sp. 16-5
Methylococcus sp. BF19-07
Methylococcus sp. EFPC2
Methylococcus sp. LS7-MC
Methylococcus sp. Mc7
Methylococcus sp.
Methylocucumis oryzae
Methylocystaceae bacterium
Methylocystaceae
Methylocystis bryophila
Methylocystis echinoides
Methylocystis heyeri
Methylocystis hirsuta
Methylocystis parvus OBBP
Methylocystis parvus
Methylocystis rosea SV97
Methylocystis rosea
Methylocystis silviterrae
Methylocystis sp. 10
Methylocystis sp. 12.2
Methylocystis sp. 16
Methylocystis sp. 2-19
Methylocystis sp. 21/1
Methylocystis sp. 2B
Methylocystis sp. 33/26
Methylocystis sp. 39
Methylocystis sp. 41
Methylocystis sp. 42/22
Methylocystis sp. 45/7a
Methylocystis sp. 48
Methylocystis sp. 494
Methylocystis sp. 50/42a
Methylocystis sp. 50/54
Methylocystis sp. 51
Methylocystis sp. 56
Methylocystis sp. 5FB1
Methylocystis sp. 5FB2
Methylocystis sp. 62/12
Methylocystis sp. 75
Methylocystis sp. 9.1
Methylocystis sp. 92.1
Methylocystis sp. 92.2
Methylocystis sp. ATCC 49242
Methylocystis sp. B16
Methylocystis sp. B2/7
Methylocystis sp. B29
Methylocystis sp. B8
Methylocystis sp. DWT
Methylocystis sp. F10V2a
Methylocystis sp. H15
Methylocystis sp. H4A
Methylocystis sp. H62
Methylocystis sp. H9a
Methylocystis sp. IMET 10484
Methylocystis sp. IMET 10486
Methylocystis sp. IMET 10489
Methylocystis sp. JTC3
Methylocystis sp. KS12
Methylocystis sp. KS30
Methylocystis sp. KS33
Methylocystis sp. KS3
Methylocystis sp. KS7
Methylocystis sp. KS8a
Methylocystis sp. KS9
Methylocystis sp. Kb7
Methylocystis sp. L43
Methylocystis sp. L6
Methylocystis sp. LR1
Methylocystis sp. LW5
Methylocystis sp. M162
Methylocystis sp. M167
Methylocystis sp. M169
Methylocystis sp. M175
Methylocystis sp. M212
Methylocystis sp. M42/1
Methylocystis sp. M6
Methylocystis sp. MJC1
Methylocystis sp. M
Methylocystis sp. NLS-7
Methylocystis sp. O14c
Methylocystis sp. Pi5/4
Methylocystis sp. SB12
Methylocystis sp. SB2
Methylocystis sp. SC2
Methylocystis sp. SD3-5
Methylocystis sp. SD5
Methylocystis sp. SE12
Methylocystis sp. SH31p
Methylocystis sp. SK28
Methylocystis sp. SM16
Methylocystis sp. SS2C
Methylocystis sp. SS37A-Re
Methylocystis sp. Sn-Cys
Methylocystis sp. WI14
Methylocystis sp. clone PVP 20P
Methylocystis sp. clone ProtK2
Methylocystis sp. enrichment culture clone Lpl4-90
Methylocystis sp. enrichment culture clone R2.5-164
Methylocystis sp. enrichment culture clone R2.F-36
Methylocystis sp. enrichment culture clone
Methylocystis sp. m1511
Methylocystis sp. m231
Methylocystis sp. m261
Methylocystis sp. ok-BEg7
Methylocystis sp. pan1
Methylocystis sp.
Methylocystis
Methyloferula stellata AR4
Methyloferula stellata
Methylogaea oryzae JCM 16910
Methylogaea oryzae
Methyloglobulus morosus KoM1
Methyloglobulus morosus
Methylohalobius crimeensis 10Ki
Methylohalobius crimeensis
Methyloligella sp.
Methylomagnum ishizawai
Methylomarinovum caldicuralii
Methylomarinum sp. SSMP1
Methylomarinum vadi
Methylomicrobium album BG8
Methylomicrobium album
Methylomicrobium lacus
Methylomicrobium sp. RS1
Methylomicrobium sp. clone ProtF04
Methylomicrobium sp. wino1
Methylomicrobium sp.
Methylomicrobium
Methylomonas albis
Methylomonas fluvii
Methylomonas koyamae JCM 16701
Methylomonas koyamae
Methylomonas lenta
Methylomonas methanica MC09
Methylomonas methanica
Methylomonas paludis
Methylomonas rapida
Methylomonas rhizoryzae
Methylomonas sp. 11b
Methylomonas sp. 83
Methylomonas sp. B2Z
Methylomonas sp. D1a
Methylomonas sp. DH-1
Methylomonas sp. EFPC1
Methylomonas sp. GYJ3
Methylomonas sp. KSPIII
Methylomonas sp. KSWIII
Methylomonas sp. Kb3
Methylomonas sp. LC 1
Methylomonas sp. LL1
Methylomonas sp. LW13
Methylomonas sp. LW15
Methylomonas sp. LW16
Methylomonas sp. LW19
Methylomonas sp. LW21
Methylomonas sp. LWB
Methylomonas sp. M5
Methylomonas sp. MK1
Methylomonas sp. ML04
Methylomonas sp. ML64
Methylomonas sp. R-45362
Methylomonas sp. R-45363
Methylomonas sp. R-45364
Methylomonas sp. R-45371
Methylomonas sp. R-45372
Methylomonas sp. R-45374
Methylomonas sp. R-45378
Methylomonas sp. R-45383
Methylomonas sp. R-49799
Methylomonas sp. R-49807
Methylomonas sp. R4F
Methylomonas sp. SM-2017
Methylomonas sp. SURF-1
Methylomonas sp. SURF-2
Methylomonas sp. Sn4
Methylomonas sp. WSC-6
Methylomonas sp. WSC-7
Methylomonas sp. ZR1
Methylomonas sp. clone PP39A
Methylomonas sp. clone PP39B
Methylomonas sp. clone PP39E
Methylomonas sp. clone PP39F
Methylomonas sp. clone PP39G
Methylomonas sp. clone PP39H
Methylomonas sp. clone PP39I
Methylomonas sp. clone ProtF01
Methylomonas sp. clone ProtK8
Methylomonas sp.
Methylomonas
Methyloparacoccus murrellii
Methylophaga thiooxydans
Methylophilus leisingeri
Methyloprofundus sedimenti
Methyloprofundus sp.
Methylorubrum populi
Methylosarcina fibrata AML-C10
Methylosarcina fibrata
Methylosarcina quisquiliarum
Methylosinus acidophilus
Methylosinus sp. 3S-1
Methylosinus sp. 69
Methylosinus sp. 8.1
Methylosinus sp. 8.2
Methylosinus sp. B3R
Methylosinus sp. B4S
Methylosinus sp. C49
Methylosinus sp. Ce-a6
Methylosinus sp. D15a
Methylosinus sp. D28
Methylosinus sp. H3A
Methylosinus sp. KRF6
Methylosinus sp. LW2
Methylosinus sp. LW3
Methylosinus sp. LW4
Methylosinus sp. M1
Methylosinus sp. ML116
Methylosinus sp. ML19
Methylosinus sp. PPN CR1
Methylosinus sp. PPN CR2
Methylosinus sp. PPN CS2
Methylosinus sp. PPN ER1
Methylosinus sp. PPN ER2
Methylosinus sp. PW1
Methylosinus sp. R-45379
Methylosinus sp. R62
Methylosinus sp. RM1
Methylosinus sp. SM-2017
Methylosinus sp. Sm6
Methylosinus sp. W3-6
Methylosinus sp. bd-1A10
Methylosinus sp. bd-1A11
Methylosinus sp. bd-1A12
Methylosinus sp. bd-1A4
Methylosinus sp. bd-1A8
Methylosinus sp. bd-1F12
Methylosinus sp. bd-1NA6
Methylosinus sp. bd-3FA2
Methylosinus sp. bd-3FA3
Methylosinus sp. bq-1A1
Methylosinus sp. bq-1A2
Methylosinus sp. eb-BF2A11
Methylosinus sp. eb-BF2A8
Methylosinus sp. eb-BF2F9
Methylosinus sp. eb-BF4F1
Methylosinus sp. enrichment culture clone Lpl2-134
Methylosinus sp. enrichment culture clone R2.3-140
Methylosinus sp. hs-BK1F13
Methylosinus sp. hs-BK1F20
Methylosinus sp. hs-BK1F21
Methylosinus sp. hs-BK1F29
Methylosinus sp. hs-BK1F6
Methylosinus sp. ht-BGNA3
Methylosinus sp. ht-BGNA6
Methylosinus sp. sav-2
Methylosinus sporium
Methylosinus trichosporium OB3b
Methylosinus trichosporium
Methylosinus
Methylosoma difficile
Methylosoma sp. TFB
Methylotenera mobilis
Methylotenera sp. GFS-K6
Methyloterricola oryzae
Methylotetracoccus oryzae
Methylothermus subterraneus
Methylothermus thermalis
Methylotuvimicrobium alcaliphilum 20Z
Methylotuvimicrobium alcaliphilum
Methylotuvimicrobium buryatense 5G
Methylotuvimicrobium buryatense
Methylotuvimicrobium japanense
Methylotuvimicrobium pelagicum
Methylotuvimicrobium
Methylovirgula sp. 4M-Z18
Methylovirgula sp. HY1
Methylovulum miyakonense HT12
Methylovulum miyakonense
Methylovulum psychrotolerans
Microbacterium hominis
Microbacterium kyungheense
Microbacterium lemovicicum
Microbacterium maritypicum
Microbacterium oxydans
Microbacterium sp. JAI119
Microbacterium sp. Leaf351
Microbacterium sp. PAMC21962
Microbacterium sp. SA39
Microbacterium sp. SLBN-146
Microbacterium trichothecenolyticum
Microbacterium
Microcella alkaliphila
Micrococcus luteus
Microcoleus asticus
Microgenomates bacterium OLB22
Microlunatus speluncae
Micromonospora endolithica
Micromonospora phaseoli
Micromonospora pisi
Micromonospora sagamiensis
Micromonospora sp. A202
Micromonospora sp. M71_S20
Micromonospora sp. MH33
Micromonospora taraxaci
Micromonospora violae
Millisia brevis
Mongoliibacter ruber
Moritella dasanensis
Moritella marina
Moritella sp. 24
Moritella sp. JT01
Moritella sp. PE36
Moritella sp. Urea-trap-13
Moritella viscosa
Moritella yayanosii
Motiliproteus coralliicola
Motiliproteus sediminis
Motiliproteus sp. MSK22-1
Motiliproteus sp. SC1-56
Mucilaginibacter auburnensis
Mucilaginibacter gotjawali
Mucilaginibacter gracilis
Mucilaginibacter oryzae
Mucilaginibacter yixingensis
Mucispirillum schaedleri
Murinocardiopsis flavida
Myceligenerans xiligouense
Mycobacteriaceae
Mycobacterium alsense
Mycobacterium aquaticum
Mycobacterium asiaticum
Mycobacterium avium
Mycobacterium botniense
Mycobacterium bourgelatii
Mycobacterium colombiense
Mycobacterium crocinum
Mycobacterium florentinum
Mycobacterium genavense
Mycobacterium goodii
Mycobacterium gordonae
Mycobacterium heraklionense
Mycobacterium hodleri
Mycobacterium holsaticum DSM 44478 = JCM 12374
Mycobacterium holsaticum
Mycobacterium intermedium
Mycobacterium intracellulare
Mycobacterium lentiflavum
Mycobacterium marinum E11
Mycobacterium marinum MB2
Mycobacterium marinum M
Mycobacterium marinum str. Europe
Mycobacterium marinum
Mycobacterium numidiamassiliense
Mycobacterium palustre
Mycobacterium paraffinicum
Mycobacterium paragordonae
Mycobacterium paraintracellulare
Mycobacterium parmense
Mycobacterium pseudoshottsii JCM 15466
Mycobacterium pseudoshottsii
Mycobacterium saskatchewanense
Mycobacterium sherrisii
Mycobacterium shigaense
Mycobacterium shimoidei
Mycobacterium shottsii
Mycobacterium simiae
Mycobacterium sp. 012931
Mycobacterium sp. 852013-50091_SCH5140682
Mycobacterium sp. AT1
Mycobacterium sp. AZCC_0083
Mycobacterium sp. Aquia_216
Mycobacterium sp. DL592
Mycobacterium sp. E1747
Mycobacterium sp. E2327
Mycobacterium sp. E2479
Mycobacterium sp. ENV421
Mycobacterium sp. ITM-2016-00317
Mycobacterium sp. JS623
Mycobacterium sp. KBS0706
Mycobacterium sp. MS1601
Mycobacterium sp. Root135
Mycobacterium sp. SM1
Mycobacterium sp. SWH-M5
Mycobacterium sp. URHB0044
Mycobacterium sp. djl-10
Mycobacterium sp. shizuoka-1
Mycobacterium sp.
Mycobacterium stomatepiae
Mycobacterium szulgai
Mycobacterium triplex
Mycobacterium tuberculosis H37RvSiena
Mycobacterium tuberculosis H37Rv
Mycobacterium tuberculosis variant bovis AF2122/97
Mycobacterium tuberculosis variant bovis BCG
Mycobacterium tuberculosis variant microti OV254
Mycobacterium tuberculosis
Mycobacterium yunnanensis
Mycobacterium
Mycolicibacter engbaekii
Mycolicibacter hiberniae
Mycolicibacter kumamotonensis
Mycolicibacter nonchromogenicus
Mycolicibacter sp. MYC017
Mycolicibacter sp. MYC098
Mycolicibacter sp. MYC340
Mycolicibacter terrae
Mycolicibacterium agri
Mycolicibacterium anyangense
Mycolicibacterium aubagnense
Mycolicibacterium austroafricanum
Mycolicibacterium brisbanense
Mycolicibacterium chubuense NBB4
Mycolicibacterium chubuense
Mycolicibacterium cosmeticum
Mycolicibacterium elephantis
Mycolicibacterium fluoranthenivorans
Mycolicibacterium hassiacum
Mycolicibacterium helvum
Mycolicibacterium hippocampi
Mycolicibacterium iranicum
Mycolicibacterium komossense
Mycolicibacterium litorale
Mycolicibacterium madagascariense
Mycolicibacterium mageritense
Mycolicibacterium mengxianglii
Mycolicibacterium moriokaense
Mycolicibacterium mucogenicum
Mycolicibacterium murale
Mycolicibacterium phlei
Mycolicibacterium pulveris
Mycolicibacterium rhodesiae JS60
Mycolicibacterium rhodesiae NBB3
Mycolicibacterium rhodesiae
Mycolicibacterium smegmatis MC2 155
Mycolicibacterium smegmatis MKD8
Mycolicibacterium smegmatis
Mycolicibacterium sp. CH28
Mycolicibacterium sp. P9-64
Mycolicibacterium sp.
Mycolicibacterium sphagni
Mycolicibacterium stellerae
Mycolicibacterium tokaiense
Mycolicibacterium tusciae
Mycolicibacterium vinylchloridicum
Mycolicibacterium xanthum
Mycolicibacterium
Myoviridae sp.
NADPH flavoprotein alpha-component Methyloceanibacter stevinii
NC10 bacterium enrichment culture clone ‘WWTP a Lieshout clone 18’
NC10 bacterium enrichment culture clone ‘WWTP a Lieshout clone 4’
NC10 bacterium enrichment culture clone ‘WWTP a Lieshout clone 6’
NC10 bacterium enrichment culture clone ‘WWTP b Lieshout clone 16’
NC10 bacterium enrichment culture clone ‘WWTP b Lieshout clone 3’
NC10 bacterium enrichment culture clone ‘WWTP b Lieshout clone 5’
NC10 bacterium enrichment culture clone ‘WWTP b Lieshout clone 6’
NC10 bacterium enrichment culture clone ‘enr a Lieshout clone 2’
NC10 bacterium enrichment culture clone ‘enr a Lieshout clone 5’
NC10 bacterium enrichment culture clone ‘enr b 332 d Lieshout clone 2’
NC10 bacterium enrichment culture clone ‘enr b Lieshout clone 10’
NC10 bacterium enrichment culture clone ‘enr b Lieshout clone 3’
NC10 bacterium enrichment culture clone ‘enr c 332 d Lieshout clone 12’
NC10 bacterium enrichment culture clone ‘enr c 332 d Lieshout clone 18’
NC10 bacterium enrichment culture clone ‘enr c Lieshout clone 5’
NC10 bacterium enrichment culture clone ‘enr d 332 d Lieshout clone 1’
NC10 bacterium enrichment culture clone ‘inoc a Lieshout clone 12’
NC10 bacterium enrichment culture clone ‘inoc a Lieshout clone 5’
NC10 bacterium enrichment culture clone ‘inoc b Lieshout clone 8’
NC10 bacterium enrichment culture clone 1 coculture 22 d
NC10 bacterium enrichment culture clone 20 coculture 87 d
NC10 bacterium enrichment culture clone 23 coculture 87 d
NC10 bacterium enrichment culture clone 32 coculture 87 d
NC10 bacterium enrichment culture clone 35 coculture 139 d
NC10 bacterium enrichment culture clone 42 coculture 139 d
NC10 bacterium enrichment culture clone 45 coculture 139 d
NC10 bacterium enrichment culture clone 9
NC10 bacterium enrichment culture clone CSTR 2-10
NC10 bacterium enrichment culture clone CSTR 2-11
NC10 bacterium enrichment culture clone CSTR 2-13
NC10 bacterium enrichment culture clone CSTR 2-15
NC10 bacterium enrichment culture clone CSTR 2-16
NC10 bacterium enrichment culture clone CSTR 2-18
NC10 bacterium enrichment culture clone CSTR 2-2
NC10 bacterium enrichment culture clone CSTR 2-3
NC10 bacterium enrichment culture clone CSTR 2-4
NC10 bacterium enrichment culture clone CSTR 2-8
NC10 bacterium enrichment culture clone FRS01
NC10 bacterium enrichment culture clone FRS02
NC10 bacterium enrichment culture clone FRS03
NC10 bacterium enrichment culture clone MGS01
NC10 bacterium enrichment culture clone MGS02
NC10 bacterium enrichment culture clone MGS03
NC10 bacterium enrichment culture clone MGS04
NC10 bacterium enrichment culture clone MGS05
NC10 bacterium enrichment culture clone MSGLR 1-10
NC10 bacterium enrichment culture clone MSGLR 1-12
NC10 bacterium enrichment culture clone MSGLR 1-14
NC10 bacterium enrichment culture clone MSGLR 1-15
NC10 bacterium enrichment culture clone MSGLR 1-16
NC10 bacterium enrichment culture clone MSGLR 1-17
NC10 bacterium enrichment culture clone MSGLR 1-18
NC10 bacterium enrichment culture clone MSGLR 1-19
NC10 bacterium enrichment culture clone MSGLR 1-2
NC10 bacterium enrichment culture clone MSGLR 1-3
NC10 bacterium enrichment culture clone MSGLR 1-4
NC10 bacterium enrichment culture clone MSGLR 1-7
NC10 bacterium enrichment culture clone MSGLR 1-8
NC10 bacterium enrichment culture clone MSGLR 1-9
NC10 bacterium enrichment culture clone MSGLR 2-1
NC10 bacterium enrichment culture clone PS01
NC10 bacterium enrichment culture clone PS02
NC10 bacterium enrichment culture
Nakamurella leprariae
Nakamurella panacisegetis
Nakamurella sp. PAMC28650
Natranaerovirga hydrolytica
Natranaerovirga pectinivora
Natrarchaeobaculum aegyptiacum
Natroniella acetigena
Natroniella sulfidigena
Natronincola peptidivorans
Natronoflexus pectinivorans
Neiella marina
Neisseria gonorrhoeae
Neolewinella xylanilytica
Neorhizobium sp. P12A
Neptunomonas antarctica
Nesterenkonia aurantiaca
Nesterenkonia sandarakina
Nevskia ramosa
Niallia endozanthoxylica
Niameybacter massiliensis
Nitratiruptor sp. SB155-2
Nitrincola sp. A-D6
Nitrincola tapanii
Nitrogeniibacter aestuarii
Nitrogeniibacter mangrovi
Nitrosarchaeum
Nitrosoarchaeum
Nitrosococcus halophilus Nc 4
Nitrosococcus halophilus
Nitrosococcus oceani AFC27
Nitrosococcus oceani ATCC 19707
Nitrosococcus oceani C-27
Nitrosococcus oceani
Nitrosococcus wardiae
Nitrosococcus watsonii C-113
Nitrosococcus watsonii
Nitrosomonas aestuarii
Nitrosomonas communis
Nitrosomonas cryotolerans ATCC 49181
Nitrosomonas cryotolerans
Nitrosomonas europaea ATCC 19718
Nitrosomonas europaea
Nitrosomonas eutropha C91
Nitrosomonas eutropha
Nitrosomonas halophila
Nitrosomonas marina
Nitrosomonas mobilis
Nitrosomonas nitrosa
Nitrosomonas oligotropha
Nitrosomonas sp. AL212
Nitrosomonas sp. ENI-11
Nitrosomonas sp. GH22
Nitrosomonas sp. H1_AOB3
Nitrosomonas sp. HPC101
Nitrosomonas sp. JL21
Nitrosomonas sp. Nm132
Nitrosomonas sp. Nm166
Nitrosomonas sp. Nm33
Nitrosomonas sp. Nm34
Nitrosomonas sp. Nm51
Nitrosomonas sp. Nm58
Nitrosomonas sp. Nm84
Nitrosomonas sp. PY1
Nitrosomonas sp. TK794
Nitrosomonas sp.
Nitrosomonas stercoris
Nitrosomonas supralitoralis
Nitrosomonas ureae
Nitrosomonas
Nitrosopumilaceae
Nitrosopumilales archaeon
Nitrosopumilus adriaticus
Nitrosopumilus cobalaminigenes
Nitrosopumilus maritimus SCM1
Nitrosopumilus maritimus
Nitrosopumilus oxyclinae
Nitrosopumilus piranensis
Nitrosopumilus sp. K4
Nitrosopumilus sp. Nsub
Nitrosopumilus sp. b1
Nitrosopumilus sp. b3
Nitrosopumilus sp.
Nitrosopumilus ureiphilus
Nitrosopumilus zosterae
Nitrosopumilus
Nitrososphaera sp. AFS
Nitrososphaera viennensis
Nitrosospira briensis C-128
Nitrosospira briensis
Nitrosospira lacus
Nitrosospira multiformis ATCC 25196
Nitrosospira multiformis
Nitrosospira sp. NRS527
Nitrosospira sp. Nl5
Nitrosospira sp. Np39-19
Nitrosospira sp. NpAV
Nitrosospira sp. Nsp11
Nitrosospira sp. Nsp13
Nitrosospira sp. Nsp14
Nitrosospira sp. Nsp18
Nitrosospira sp. Nsp1
Nitrosospira sp. Nsp2
Nitrosospira sp. Nsp5
Nitrosospira sp.
Nitrosospira
Nitrosovibrio sp. Nv17
Nitrosovibrio sp. Nv4
Nitrosovibrio sp. Nv6
Nitrosovibrio tenuis
Nitrospira cf. moscoviensis SBR1015
Niveibacterium microcysteis
Niveibacterium sp. COAC-50
Niveibacterium
Nocardia africana
Nocardia altamirensis
Nocardia aobensis
Nocardia coffeae
Nocardia colli
Nocardia coubleae
Nocardia donostiensis
Nocardia farcinica
Nocardia jiangxiensis
Nocardia miyunensis
Nocardia niigatensis
Nocardia nova
Nocardia paucivorans
Nocardia sienata
Nocardia sp. BSTN01
Nocardia sp. DSM 111727
Nocardia sp. ncl2
Nocardia transvalensis
Nocardia vaccinii
Nocardia
Nocardioidaceae bacterium Broad-1
Nocardioides dokdonensis FR1436
Nocardioides dokdonensis
Nocardioides donggukensis
Nocardioides immobilis
Nocardioides jensenii
Nocardioides luteus
Nocardioides sp. CF8
Nocardioides sp. JS614
Nocardioides sp. Root140
Nocardioides sp. Root151
Nocardioides sp. Root614
Nocardioides sp. Root682
Nocardioides sp. Soil796
Nocardioides sp. dk884
Nocardioides sp. zg-DK7169
Nocardiopsis sp. Huas11
Nodosilinea nodulosa
Nonomuraea basaltis
Nonomuraea fuscirosea
Nonomuraea guangzhouensis
Nonomuraea polychroma
Nonomuraea rhizosphaerae
Nonomuraea solani
Nonomuraea sp. NEAU-A123
Nonomuraea sp. PA05
Nonomuraea thailandensis
Nonomuraea zeae
Noviherbaspirillum aridicola
Noviherbaspirillum autotrophicum
Noviherbaspirillum denitrificans
Noviherbaspirillum sp. UKPF54
Novispirillum itersonii
Novisyntrophococcus fermenticellae
Novosphingobium sp. 63-713
Novosphingobium sp. Fuku2-ISO-50
Novosphingobium sp. PASSN1
Novosphingobium sp. SCN 63-17
Novosphingobium sp. SG707
Occallatibacter riparius
Occallatibacter savannae
Oceanidesulfovibrio indonesiensis
Oceanidesulfovibrio marinus
Oceanispirochaeta crateris
Oceanococcus atlanticus
Oharaeibacter diazotrophicus
Oleidesulfovibrio alaskensis
Oleiphilus messinensis
Oleomonas cavernae
Orenia marismortui
Orenia metallireducens
Orrella marina
Oryzomicrobium terrae
Oryzomonas japonica
Oryzomonas rubra
Oryzomonas sagensis
Oryzomonas
Oscillatoria sp. FACHB-1407
Otoolea sp. DSM 111138
Ottowia oryzae
Oxalobacter formigenes
Oxobacter pfennigii
Paenibacillus azotifigens
Paenibacillus borealis
Paenibacillus durus
Paenibacillus forsythiae
Paenibacillus jilunlii
Paenibacillus odorifer
Paenibacillus piscarius
Paenibacillus rhizophilus
Paenibacillus silagei
Paenibacillus sonchi group
Paenibacillus sonchi
Paenibacillus sophorae
Paenibacillus sp. FSL H7-0357
Paenibacillus sp. FSL R5-0912
Paenibacillus sp. FSL R7-0337
Paenibacillus sp. FSL R7-269
Paenibacillus sp. FSL R7-277
Paenibacillus sp. JJ-60
Paenibacillus sp. MMS18-CY102
Paenibacillus sp. NFR01
Paenibacillus sp. P46E
Paenibacillus sp. PK3_47
Paenibacillus sp. S150
Paenibacillus sp. VTT E-133280
Paenibacillus sp. VTT E-133291
Paenibacillus sp. YN15
Paenibacillus stellifer
Paenibacillus taihuensis
Paenibacillus tengchongensis
Paenibacillus tepidiphilus
Paenibacillus zanthoxyli
Paenibacillus
Paeniclostridium hominis
Paeniclostridium sordellii
Paenisporosarcina antarctica
Paludibacterium purpuratum
Paludibaculum fermentans
Paludisphaera rhizosphaereae
Panacagrimonas perspica
Pandoraea terrae
Paraburkholderia acidiphila
Paraburkholderia caffeinitolerans
Paraburkholderia caribensis MBA4
Paraburkholderia caribensis
Paraburkholderia dinghuensis
Paraburkholderia hiiakae
Paraburkholderia hospita
Paraburkholderia madseniana
Paraburkholderia monticola
Paraburkholderia piptadeniae
Paraburkholderia tropica
Paraburkholderia ultramafica
Paraburkholderia unamae
Paraclostridium benzoelyticum
Paraclostridium bifermentans
Paraclostridium dentum
Paraclostridium sp. AKS73
Paracoccaceae bacterium
Paracoccus haematequi
Paracoccus sp. IB05
Paraconexibacter algicola
Paraconexibacter antarcticus
Paradesulfovibrio bizertensis
Paramaledivibacter caminithermalis
Parapedobacter indicus
Pararhodobacter aggregans
Pararhodospirillum oryzae
Pararhodospirillum photometricum
Parasulfuritortus cantonensis
Parazoarcus communis
Parcubacteria bacterium OLB19
Parvivirga hydrogeniphila
Patescibacteria group bacterium
Patulibacter medicamentivorans
Paucibacter sp. KBW04
Paucibacter sp. KCTC 42545
Paucibacter sp. hw8
Paucidesulfovibrio gracilis
Paucidesulfovibrio longus
Pectinatus brassicae
Pectinatus frisingensis
Pedobacter metabolipauper
Pelagibaculum spongiae
Pelagicoccus albus
Pelagicoccus mobilis
Pelagimonas varians
Pelobacter propionicus
Pelobacter seleniigenes
Pelorhabdus rhamnosifermentans
Pelosinus fermentans
Pelosinus propionicus
Pelosinus sp. Bkl1
Pelosinus sp. UFO1
Pelosinus
Pelovirga terrestris
Peptoclostridium acidaminophilum
Peptoclostridium litorale
Perlabentimonas gracilis
Permianibacter aggregans
Permianibacter fluminis
Persephonella atlantica
Persephonella hydrogeniphila
Persephonella marina
Persephonella sp. KM09-Lau-8
Petrocella atlantisensis
Petroclostridium xylanilyticum
Phaeobacter sp. CECT 5382
Phenylobacterium sp. Root700
Phormidium sp. FACHB-1136
Photobacterium frigidiphilum
Phycicoccus sp. HDW14
Pinisolibacter aquiterrae
Piscicoccus intestinalis
Piscirickettsia salmonis
Planktothrix mougeotii
Planktothrix sp. FACHB-1365
Planktothrix
Planosporangium flavigriseum
Planosporangium mesophilum
Plantibacter flavus
Plasmodium yoelii yoelii
Plasticicumulans acidivorans
Plasticicumulans lactativorans
Pleomorphomonas diazotrophica
Pleomorphomonas koreensis
Pleomorphomonas oryzae
Pleomorphomonas sp. JP5
Pleomorphomonas sp. NRK KF1
Pleomorphomonas sp. SM30
Polaromonas sp. 24-62-144
Polaromonas sp. 28-63-22
Polaromonas sp. 35-63-240
Polaromonas sp. 39-63-203
Polaromonas sp. AER18D-145
Polaromonas sp. AET17H-212
Polynucleobacter cosmopolitanus
Pontibacterium sp. N1Y112
Ponticaulis koreensis
Ponticoccus alexandrii
Pontiella desulfatans
Pontiella sulfatireligans
Poseidonibacter antarcticus
Poseidonibacter lekithochrous
Poseidonibacter parvus
Poseidonibacter sp. JOD-M-6
Poseidonibacter sp. SJOD-M-5
Prauserella aidingensis
Prauserella alba
Prauserella cavernicola
Prauserella coralliicola
Prauserella endophytica
Prauserella flavalba
Prauserella muralis
Prauserella sediminis
Prauserella sp. PE36
Prauserella
Prochlorothrix hollandica
Promicromonospora sp. AC04
Propionispira arboris
Propionispira raffinosivorans
Propionispira
Propionispora hippei
Propionispora sp. 2/2-37
Propionispora vibrioides
Propionivibrio dicarboxylicus
Propionivibrio limicola
Propionivibrio sp. SG131
Prosthecobacter fusiformis
Proteobacteria bacterium SG_bin4
Pseudaminobacter arsenicus
Pseudanabaena biceps
Pseudanabaena sp. ‘Roaring Creek’
Pseudanabaena sp. ABRG5-3
Pseudanabaena sp. BC1403
Pseudanabaena sp. Chao 1811
Pseudanabaena sp. FACHB-1050
Pseudanabaena sp. FACHB-1998
Pseudanabaena sp. FACHB-723
Pseudanabaena sp. SR411
Pseudanabaena sp. UWO310
Pseudanabaena sp. UWO311
Pseudanabaena sp. lw0831
Pseudanabaena yagii
Pseudemcibacter aquimaris
Pseudoalteromonas denitrificans
Pseudoalteromonas sp. C2R02
Pseudobacter ginsenosidimutans
Pseudobacteriovorax antillogorgiicola
Pseudoclostridium thermosuccinogenes
Pseudodesulfovibrio aespoeensis
Pseudodesulfovibrio alkaliphilus
Pseudodesulfovibrio cashew
Pseudodesulfovibrio hydrargyri
Pseudodesulfovibrio indicus
Pseudodesulfovibrio mercurii
Pseudodesulfovibrio piezophilus
Pseudodesulfovibrio portus
Pseudodesulfovibrio profundus
Pseudodesulfovibrio sediminis
Pseudodesulfovibrio senegalensis
Pseudodesulfovibrio sp. JC047
Pseudodesulfovibrio sp. MCM B-1480
Pseudodesulfovibrio sp. zrk46
Pseudodesulfovibrio tunisiensis
Pseudoflavonifractor phocaeensis
Pseudolysinimonas yzui
Pseudomaricurvus alkylphenolicus
Pseudomonadota bacterium
Pseudomonas aeruginosa DK1
Pseudomonas aeruginosa
Pseudomonas alcaligenes
Pseudomonas alkylphenolica
Pseudomonas anguilliseptica
Pseudomonas benzenivorans
Pseudomonas borbori
Pseudomonas cavernae
Pseudomonas cavernicola
Pseudomonas citronellolis
Pseudomonas fluorescens
Pseudomonas fluvialis
Pseudomonas kurunegalensis
Pseudomonas pharmacofabricae
Pseudomonas resinovorans
Pseudomonas saudiphocaensis
Pseudomonas sp. 1-7
Pseudomonas sp. C27(2019)
Pseudomonas sp. GW704-F2
Pseudomonas sp. GW704-F3
Pseudomonas sp. GW704-F5
Pseudomonas sp. MIL19
Pseudomonas sp. MPBD4-3
Pseudomonas sp. P42
Pseudomonas sp. PDNC002
Pseudomonas sp. TTU2014-080ASC
Pseudomonas sp. phDV1
Pseudomonas yangmingensis
Pseudonocardia abyssalis
Pseudonocardia acaciae
Pseudonocardia acidicola
Pseudonocardia alaniniphila
Pseudonocardia alni
Pseudonocardia ammonioxydans
Pseudonocardia asaccharolytica DSM 44247 = NBRC 16224
Pseudonocardia asaccharolytica
Pseudonocardia autotrophica
Pseudonocardia bannensis
Pseudonocardia broussonetiae
Pseudonocardia cypriaca
Pseudonocardia dioxanivorans CB1190
Pseudonocardia dioxanivorans
Pseudonocardia endophytica
Pseudonocardia halophobica
Pseudonocardia hierapolitana
Pseudonocardia hydrocarbonoxydans
Pseudonocardia kujensis
Pseudonocardia kunmingensis
Pseudonocardia nigra
Pseudonocardia oceani
Pseudonocardia oroxyli
Pseudonocardia parietis
Pseudonocardia petroleophila
Pseudonocardia pini
Pseudonocardia saturnea
Pseudonocardia sediminis
Pseudonocardia sp. AL041005-10
Pseudonocardia sp. Ae150A_Ps1
Pseudonocardia sp. Ae168_Ps1
Pseudonocardia sp. Ae263_Ps1
Pseudonocardia sp. Ae331_Ps2
Pseudonocardia sp. Ae356_Ps1
Pseudonocardia sp. Ae406_Ps2
Pseudonocardia sp. Ae505_Ps2
Pseudonocardia sp. Ae706_Ps2
Pseudonocardia sp. Ae707_Ps1
Pseudonocardia sp. Ae717_Ps2
Pseudonocardia sp. C8
Pseudonocardia sp. CNS-139
Pseudonocardia sp. DSM 110487
Pseudonocardia sp. EC080610-09
Pseudonocardia sp. EC080619-01
Pseudonocardia sp. EC080625-04
Pseudonocardia sp. EV170527-09
Pseudonocardia sp. H11422
Pseudonocardia sp. H11425
Pseudonocardia sp. HH130629-09
Pseudonocardia sp. HH130630-07
Pseudonocardia sp. ICBG1293
Pseudonocardia sp. ICBG162
Pseudonocardia sp. KRD291
Pseudonocardia sp. MH-G8
Pseudonocardia sp. N23
Pseudonocardia sp. S2-4
Pseudonocardia sp. SCN 73-27
Pseudonocardia sp. SID8383
Pseudonocardia sp. TRM90224
Pseudonocardia sp. WMMC193
Pseudonocardia spinosispora
Pseudonocardia sulfidoxydans NBRC 16205
Pseudonocardia sulfidoxydans
Pseudonocardia terrae
Pseudonocardia thermophila
Pseudonocardia xinjiangensis
Pseudonocardia
Pseudonocardiaceae bacterium YIM PH 21723
Pseudonocardiaceae bacterium
Pseudonocardiaceae
Pseudooceanicola lipolyticus
Pseudothauera hydrothermalis
Pseudothauera lacus
Pseudothauera nasutitermitis
Pseudothauera rhizosphaerae
Pseudovibrio exalbescens
Psychrobacter sanguinis
Psychrobacter sp. SC65A.3
Psychrodesulfovibrio sp. FT415
Psychromonas antarctica
Psychromonas aquimarina
Psychromonas ossibalaenae
Pusillimonas caeni
Pygmaiobacter massiliensis
Pyrobaculum
Pyrodictium delaneyi
Pyrodictium occultum
Pyrolobus fumarii
Qaidamihabitans albus
Quatrionicoccus australiensis
Raineyella antarctica
Raineyella fluvialis
Ralstonia pickettii DTP0602
Ralstonia sp. PaPiEm3-17
Ralstonia sp. PaPiMm3-1
Ralstonia sp. PaPiMm3-6
Ramlibacter alkalitolerans
Ramlibacter lithotrophicus
Ramlibacter monticola
Ramlibacter sp. B156
Ramlibacter sp. GTP1
Ramlibacter sp. WS9
Rathayibacter sp. Leaf294
Rathayibacter sp. PhB127
Rathayibacter sp. PhB151
Rathayibacter sp. PhB152
Rathayibacter sp. PhB179
Rathayibacter sp. PhB185
Rathayibacter sp. PhB186
Rathayibacter sp. PhB192
Rathayibacter sp. PhB1
Rathayibacter sp. PhB93
Reichenbachiella sp. ABR2-5
Reyranella sp. SM-2017
Rhabdochromatium marinum
Rhizobium altiplani
Rhizobium jaguaris
Rhizobium sp. AgNmSr5-1
Rhizobium sp. AgNmUh5-1
Rhizobium sp. C1
Rhizobium sp. PaPiEm3-1
Rhizobium sp.
Rhizocola hellebori
Rhizomicrobium electricum
Rhizomicrobium palustre
Rhodobacter sp. AKP1
Rhodobacteraceae bacterium PD-2
Rhodobacterales bacterium 65-51
Rhodobacterales bacterium RIFCSPHIGHO2_02_FULL_62_130
Rhodobacterales bacterium RIFCSPHIGHO2_12_FULL_62_75
Rhodobium gokarnense
Rhodobium orientis
Rhodoblastus acidophilus
Rhodococcus aetherivorans
Rhodococcus coprophilus
Rhodococcus erythropolis
Rhodococcus fascians D188
Rhodococcus fascians
Rhodococcus jostii
Rhodococcus koreensis
Rhodococcus kyotonensis
Rhodococcus marinonascens
Rhodococcus opacus M213
Rhodococcus opacus PD630
Rhodococcus opacus RKJ300 = JCM 13270
Rhodococcus opacus
Rhodococcus oxybenzonivorans
Rhodococcus pseudokoreensis
Rhodococcus pyridinivorans
Rhodococcus qingshengii
Rhodococcus rhodochrous ATCC 21198
Rhodococcus rhodochrous KG-21
Rhodococcus rhodochrous
Rhodococcus ruber BKS 20-38
Rhodococcus ruber Chol-4
Rhodococcus ruber
Rhodococcus sp. 008
Rhodococcus sp. 05-2254-1
Rhodococcus sp. 05-2254-5
Rhodococcus sp. 05-2255-1e
Rhodococcus sp. 05-2256-B1
Rhodococcus sp. 05-2256-B2
Rhodococcus sp. 05-2256-B3
Rhodococcus sp. 05-2256-B4
Rhodococcus sp. 06-221-2
Rhodococcus sp. 11-3
Rhodococcus sp. 14C212
Rhodococcus sp. 21391
Rhodococcus sp. ACPA1
Rhodococcus sp. ACS1
Rhodococcus sp. B50
Rhodococcus sp. CH91
Rhodococcus sp. DK17
Rhodococcus sp. DMF-1
Rhodococcus sp. DMU1
Rhodococcus sp. ENV425
Rhodococcus sp. EPR-157
Rhodococcus sp. Eu-32
Rhodococcus sp. FXJ9.536
Rhodococcus sp. JS3073
Rhodococcus sp. JVH1
Rhodococcus sp. KBS0724
Rhodococcus sp. KRD197
Rhodococcus sp. LB1
Rhodococcus sp. Leaf233
Rhodococcus sp. M8
Rhodococcus sp. MS16
Rhodococcus sp. MSC1_016
Rhodococcus sp. NCIMB 12038
Rhodococcus sp. NKCM2511
Rhodococcus sp. OK302
Rhodococcus sp. PSBB049
Rhodococcus sp. PSBB066
Rhodococcus sp. SC4
Rhodococcus sp. T7
Rhodococcus sp. USK10
Rhodococcus sp. USK13
Rhodococcus sp. W8901
Rhodococcus sp. WAY2
Rhodococcus sp. WB1
Rhodococcus sp. WB9
Rhodococcus sp. WMMA185
Rhodococcus sp. WS4
Rhodococcus sp. ZPP
Rhodococcus sp.
Rhodococcus wratislaviensis IFP 2016
Rhodococcus wratislaviensis
Rhodococcus yunnanensis
Rhodococcus zopfii
Rhodococcus
Rhodocyclaceae bacterium
Rhodocyclus gracilis
Rhodocyclus tenuis
Rhodoferax antarcticus
Rhodoferax fermentans
Rhodoferax ferrireducens
Rhodoferax lacus
Rhodoferax sp. BAB1
Rhodoferax sp. BLA1
Rhodoferax sp. MIZ03
Rhodoferax sp. PAMC 29310
Rhodoferax sp. U11-2br
Rhodoferax sp. U2-2l
Rhodomicrobium lacus
Rhodopseudomonas palustris
Rhodopseudomonas rhenobacensis
Rhodopseudomonas sp. AAP120
Rhodopseudomonas sp. AgNmSr5-2
Rhodopseudomonas sp. BR0M22
Rhodopseudomonas
Rhodospira trueperi
Rhodospirillaceae bacterium
Rhodospirillales bacterium 20-60-12
Rhodospirillum rubrum
Rhodovastum atsumiense
Rhodovulum sulfidophilum
Richelia sinica
Robinsoniella peoriensis
Robinsoniella
Romboutsia lituseburensis
Romboutsia maritimum
Roseburia intestinalis
Roseburia inulinivorans
Roseburia lenta
Roseburia sp. 1XD42-69
Roseburia sp. 499
Roseburia sp. AF12-17LB
Roseburia sp. AF15-21
Roseburia sp. AM16-25
Roseburia sp. AM23-20
Roseburia sp. BX1005
Roseburia sp. MSJ-14
Roseburia sp. NSJ-69
Roseburia sp. OF03-24
Roseburia
Roseibium album
Roseimicrobium gellanilyticum
Roseobacter sp. TSBP12
Roseospira goensis
Roseospira marina
Roseospira navarrensis
Roseospira visakhapatnamensis
Roseospirillum parvum
Roseovarius aestuarii
Roseovarius litorisediminis
Rubidibacter lacunae
Rubrivivax sp. A210
Rubrobacter aplysinae
Rubrobacter indicoceani
Rubrobacter tropicus
Ruegeria atlantica
Ruegeria sp. PBVC088
Ruegeria sp. PrR005
Rugosimonospora africana
Ruminiclostridium cellobioparum
Ruminiclostridium cellulolyticum
Ruminiclostridium hungatei
Ruminiclostridium josui
Ruminiclostridium papyrosolvens
SAR324 cluster bacterium
Saccharicrinis fermentans
Saccharobesus litoralis
Saccharomonospora amisosensis
Saccharomonospora halophila
Saccharomonospora iraqiensis
Saccharomonospora marina
Saccharomonospora
Saccharopolyspora aridisoli
Saccharopolyspora endophytica
Saccharopolyspora erythraea NRRL 2338
Saccharopolyspora gloriosae
Saccharopolyspora pogona
Saccharopolyspora shandongensis
Saccharopolyspora sp. ASAGF58
Saccharopolyspora sp. HNM0983
Saccharopolyspora sp. K220
Saccharopolyspora sp. NFXS83
Saccharopolyspora sp. WRP15-2
Saccharopolyspora terrae
Saccharothrix carnea
Saccharothrix saharensis
Saccharothrix texasensis
Salegentibacter salegens
Salidesulfovibrio brasiliensis
Salidesulfovibrio onnuriiensis
Salinispira pacifica
Salinispora arenicola
Salinivibrio costicola
Salinivibrio kushneri
Salinivibrio proteolyticus
Salinivibrio sharmensis
Salinivibrio siamensis
Salinivibrio socompensis
Salinivibrio sp. BNH
Salinivibrio sp. DV
Salinivibrio sp. ES.052
Salinivibrio sp. IB574
Salinivibrio sp. IB872
Salinivibrio sp. MA607
Salinivibrio sp. PR5
Salinivibrio
Salinivirga cyanobacteriivorans
Scatolibacter rhodanostii
Schinkia azotoformans
Schnuerera sp. xch1
Schnuerera ultunensis
Sciscionella marina
Sedimentibacter acidaminivorans
Sedimentibacter hydroxybenzoicus
Sedimentibacter saalensis
Sedimentibacter sp. B4
Sedimenticola selenatireducens
Sedimenticola sp. SB48
Sedimenticola thiotaurini
Sediminibacterium goheungense
Sediminibacterium magnilacihabitans
Sediminihabitans luteus
Sediminispirochaeta bajacaliforniensis
Sediminispirochaeta smaragdinae
Selenihalanaerobacter shriftii
Seleniivibrio woodruffii
Selenomonas bovis
Selenomonas noxia
Selenomonas sp. oral taxon 478
Selenomonas sputigena ATCC 35185
Selenomonas sputigena
Sellimonas intestinalis
Senegalia massiliensis
Seramator thermalis
Serpentinicella alkaliphila
Serratia fonticola
Shewanella aestuarii
Shewanella benthica
Shewanella gaetbuli
Shewanella glacialimarina
Shewanella glacialipiscicola
Shewanella psychropiezotolerans
Shewanella sp. FJAT-53870
Shewanella sp. FYR11-62
Shewanella sp. SM32
Shewanella sp. SP1S2-7
Shewanella sp. UCD-KL12
Shewanella sp. YLB-07
Shewanella
Siculibacillus lacustris
Sideroxyarcus emersonii
Sideroxydans lithotrophicus
Silanimonas lenta
Simiaoa sunii
Simplicispira hankyongi
Simplicispira metamorpha
Simplicispira psychrophila
Sinanaerobacter chloroacetimidivorans
Sinimarinibacterium sp. CAU 1509
Sinirhodobacter huangdaonensis
Siphoviridae sp.
Skermanella aerolata KACC 11604
Skermanella aerolata
Skermanella mucosa
Skermanella rosea
Skermanella sp. TT6
Skermanella stibiiresistens SB22
Skermanella stibiiresistens
Smaragdicoccus niigatensis
Sneathiella limimaris
Sneathiella litorea
Snodgrassella sp. CFCC 13594
Solemya elarraichensis gill symbiont
Solemya pervernicosa gill symbiont
Solemya velesiana gill symbiont
Solemya velum gill symbiont
Solidesulfovibrio aerotolerans
Solidesulfovibrio alcoholivorans
Solidesulfovibrio carbinolicus
Solidesulfovibrio carbinoliphilus
Solidesulfovibrio fructosivorans
Solidesulfovibrio magneticus
Solimonas aquatica
Solimonas soli
Solirubrobacter ginsenosidimutans
Solirubrobacter pauli
Solirubrobacter phytolaccae
Solirubrobacter sp. CPCC 204708
Solirubrobacter sp. URHD0082
Solirubrobacter taibaiensis
Sorangium sp. Soce836
Sphaerospermopsis aphanizomenoides
Sphaerospermopsis sp. LEGE 00249
Sphaerospermopsis sp. LEGE 08334
Sphaerotilus hippei
Sphaerotilus sp. FB-5
Sphingobacterium detergens
Sphingobium sp. SCG-1
Sphingobium sp. TKS
Sphingobium yanoikuyae
Sphingomonadales bacterium
Sphingomonas sp. AAP5
Sphingomonas
Sphingopyxis sp. OPL5
Sphingosinithalassobacter tenebrarum
Spirochaeta africana
Spirochaeta cellobiosiphila
Spirochaeta isovalerica
Spirochaeta lutea
Spirosoma oryzae
Spongiibacter thalassae
Sporanaerobacter acetigenes
Sporobacter termitidis
Sporohalobacter salinus
Sporolituus thermophilus
Sporomusa ovata
Sporomusa silvacetica
Sporomusaceae
Sporosalibacterium faouarense
Staphylococcus aureus
Staphylococcus lugdunensis
Starkeya novella
Starkeya sp. ORNL1
Stenotrophomonas maltophilia
Stenotrophomonas sp. PaPiEm3-16
Stenotrophomonas sp. PaPiEm3-7
Stenotrophomonas sp. PaPiEm3-8
Steroidobacter agaridevorans
Steroidobacter cummioxidans
Steroidobacter gossypii
Stieleria neptunia
Streptococcus agalactiae
Streptococcus dysgalactiae
Streptococcus oralis
Streptomyces acidiscabies
Streptomyces avidinii
Streptomyces blattellae
Streptomyces brasiliensis
Streptomyces brevispora
Streptomyces canus
Streptomyces capillispiralis
Streptomyces carpinensis
Streptomyces cellulosae
Streptomyces cocklensis
Streptomyces coelicolor A3(2)
Streptomyces coelicolor
Streptomyces dioscori
Streptomyces endocoffeicus
Streptomyces humicola
Streptomyces ipomoeae
Streptomyces ochraceiscleroticus
Streptomyces plumbidurans
Streptomyces puniciscabiei
Streptomyces scabiei
Streptomyces sp. 2221.1
Streptomyces sp. 7R016
Streptomyces sp. 840.1
Streptomyces sp. 846.5
Streptomyces sp. 8L
Streptomyces sp. ADI95-17
Streptomyces sp. Ag109_O5-1
Streptomyces sp. Ag82_O1-15
Streptomyces sp. Amel2xB2
Streptomyces sp. BK022
Streptomyces sp. BK205
Streptomyces sp. BK208
Streptomyces sp. BK239
Streptomyces sp. BK340
Streptomyces sp. CEV 2-1
Streptomyces sp. CRSS-Y-16
Streptomyces sp. DSM 40750
Streptomyces sp. DpondAA-A50
Streptomyces sp. DpondAA-E10
Streptomyces sp. DvalAA-21
Streptomyces sp. GMR22
Streptomyces sp. HB202
Streptomyces sp. KS 21
Streptomyces sp. KhCrAH-43
Streptomyces sp. NEAU-383
Streptomyces sp. NRRL B-24720
Streptomyces sp. NRRL F-525
Streptomyces sp. NRRL S-813
Streptomyces sp. PanSC19
Streptomyces sp. PsTaAH-137
Streptomyces sp. RB17
Streptomyces sp. RCU064
Streptomyces sp. Ru73
Streptomyces sp. S4.7
Streptomyces sp. SID13031
Streptomyces sp. SID14478
Streptomyces sp. SLBN-115
Streptomyces sp. SLBN-134
Streptomyces sp. SLBN-31
Streptomyces sp. T12
Streptomyces sp. TLI_185
Streptomyces sp. TLI_55
Streptomyces sp. TP-A0356
Streptomyces sp. TRM68367
Streptomyces sp. VMFN-G11Ma
Streptomyces sp. WM6386
Streptomyces violens
Streptomyces xylophagus
Stutzerimonas stutzeri
Sulfobacillus acidophilus DSM 10332
Sulfobacillus acidophilus TPY
Sulfobacillus benefaciens
Sulfuricaulis limicola
Sulfuricella denitrificans
Sulfuricella sp. T08
Sulfurimicrobium lacus
Sulfurimonas autotrophica
Sulfurimonas denitrificans
Sulfurimonas gotlandica
Sulfurimonas hongkongensis
Sulfurimonas lithotrophica
Sulfurimonas marina
Sulfurisoma sediminicola
Sulfuritalea hydrogenivorans
Sulfurivermis fontis
Sulfurospirillum arcachonense
Sulfurospirillum arsenophilum
Sulfurospirillum sp. DNRA8
Sulfurospirillum sp. UCH001
Sulfurovum indicum
Sulfurovum sp. NBC37-1
Sunxiuginia elliptica
Suttonella ornithocola
Suttonella sp. R2A3
Synechocystis
Synechocystis salina
Syntrophobotulus glycolicus
Syntrophomonas palmitatica
Syntrophomonas wolfei
Syntrophomonas zehnderi
Syntrophotalea carbinolica
Tardiphaga sp. vice154
Tardiphaga sp. vice278
Tardiphaga sp. vice304
Tardiphaga sp. vice352
Telmatospirillum siberiense
Telmatospirillum sp. J64-1
Tenuifilum thalassicum
Tepidanaerobacter acetatoxydans
Tepidibacillus fermentans
Tepidimicrobium xylanilyticum
Tepidimonas sediminis
Tepidiphilus succinatimandens
Terasakiella brassicae
Terasakiella pusilla
Terasakiella sp. SH-1
Teredinibacter haidensis
Terrabacter sp. MAHUQ-38
Terrisporobacter mayombei
Thalassobaculum fulvum
Thalassobius mangrovi
Thalassolituus marinus
Thalassomonas actiniarum
Thalassomonas haliotis
Thalassomonas viridans
Thalassotalea algicola
Thalassotalea piscium
Thalassotalea profundi
Thalassotalea sp. G2M2-11
Thalassotalea sp. ND16A
Thauera aminoaromatica
Thauera butanivorans
Thauera linaloolentis
Thauera phenylacetica
Thauera propionica
Thauera sp. CAU 1555
Thauera sp. K11
Thauera sp. MZ1T
Thauera sp. SWB20
Thauera sp. Sel9
Thauera terpenica
Thauera
Thaumarchaeota archaeon
Thermanaerothrix daxensis
Thermasporomyces composti
Thermincola ferriacetica
Thermincola potens
Thermincola
Thermoactinomyces mirandus
Thermoanaerobacter italicus
Thermoanaerobacter kivui
Thermoanaerobacter mathranii
Thermoanaerobacter siderophilus
Thermoanaerobacter sp. A7A
Thermoanaerobacter sp. CM-CNRG TB177
Thermoanaerobacter sp. RKWS2
Thermoanaerobacter sp. YS13
Thermoanaerobacter thermocopriae
Thermoanaerobacter thermohydrosulfuricus
Thermoanaerobacter uzonensis
Thermoanaerobacter
Thermoanaerobacterium butyriciformans
Thermoanaerobacterium saccharolyticum
Thermoanaerobacterium sp. PSU-2
Thermoanaerobacterium sp. RBIITD
Thermoanaerobacterium thermosaccharolyticum
Thermoanaerobacterium xylanolyticum
Thermoanaerobacterium
Thermobrachium celere
Thermoclostridium caenicola
Thermoclostridium stercorarium
Thermodesulfatator atlanticus
Thermodesulfatator autotrophicus
Thermodesulfatator indicus
Thermodesulfobacterium commune
Thermodesulfobacterium geofontis
Thermodesulfobacterium hveragerdense
Thermodesulfobacterium hydrogeniphilum
Thermodesulfobacterium sp. TA1
Thermodesulfobacterium thermophilum
Thermodesulfobium acidiphilum
Thermodesulfobium narugense
Thermodesulforhabdus norvegica
Thermogemmatispora tikiterensis
Thermohalobacter berrensis
Thermomicrobium roseum
Thermophagus xiamenensis
Thermosediminibacter litoriperuensis
Thermosediminibacter oceani
Thermospira aquatica
Thermosulfurimonas marina
Thermosulfuriphilus ammonigenes
Thermosyntropha lipolytica
Thermotalea metallivorans
Thioalbus denitrificans
Thioalkalivibrio sp. ALJ15
Thioalkalivibrio sp. ALJ2
Thioalkalivibrio sp. ALJ3
Thiobaca trueperi
Thiocapsa bogorovii
Thiocapsa imhoffii
Thiocapsa marina
Thiocapsa rosea
Thiocapsa roseopersicina
Thiocapsa sp. KS1
Thiocystis violacea
Thioflexithrix psekupsensis
Thiogranum longum
Thiohalocapsa halophila
Thiohalocapsa sp. ML1
Thiohalocapsa sp. PB-PSB1
Thiohalophilus thiocyanatoxydans
Thiolapillus brandeum
Thiolinea disciformis
Thiomonas sp. FB-Cd
Thiorhodococcus drewsii
Thiorhodococcus mannitoliphagus
Thiorhodococcus minor
Thiospirillum jenense
Thiospirochaeta perfilievii
Thiothrix eikelboomii
Tichowtungia aerotolerans
Tissierella carlieri
Tissierella praeacuta
Tissierella simiarum
Tissierella sp. P1
Tolumonas auensis
Tolumonas lignilytica
Tolumonas osonensis
Trebonia kvetii
Treponema berlinense
Treponema brennaborense
Treponema denticola
Treponema medium
Treponema parvum
Treponema pedis
Treponema peruense
Treponema phagedenis
Treponema porcinum
Treponema primitia
Treponema sp. J25
Treponema sp. JC4
Treponema sp. Marseille-Q3903
Treponema sp. Marseille-Q4132
Treponema sp. OMZ 305
Treponema sp. OMZ 803
Treponema sp. OMZ 855
Treponema sp. OMZ 857
Treponema succinifaciens
Treponema vincentii
Treponema zioleckii
Treponema zuelzerae
Trichlorobacter lovleyi
Trichlorobacter thiogenes
Tulosesus angulatus
Turneriella parva
Uliginosibacterium aquaticum
Uliginosibacterium gangwonense
Uliginosibacterium sp. TH139
Uliginosibacterium
Undibacterium amnicola
Undibacterium aquatile
Undibacterium baiyunense
Undibacterium fentianense
Undibacterium flavidum
Undibacterium macrobrachii
Undibacterium rivi
Undibacterium seohonense
Undibacterium sp. FT79W
Undibacterium umbellatum
Undibacterium
Vallitalea guaymasensis
Vallitalea pronyensis
Variimorphobacter saccharofermentans
Variovorax sp. E3
Variovorax sp. PBL-H6
Varunaivibrio sulfuroxidans
Verminephrobacter eiseniae EF01-2
Verminephrobacter sp. Larva24
Verrucomicrobia bacterium ADurb.Bin018
Verrucomicrobium sp. 3C
Vibrio aerogenes
Vibrio aestuarianus
Vibrio albus
Vibrio barjaei
Vibrio campbellii
Vibrio cholerae
Vibrio fortis
Vibrio gazogenes
Vibrio hyugaensis
Vibrio maerlii
Vibrio mangrovi
Vibrio mediterranei
Vibrio ponticus
Vibrio quintilis
Vibrio rhizosphaerae
Vibrio sinensis
Vibrio sp. 99-8-1
Vibrio sp. A621
Vibrio sp. B1ASS3
Vibrio sp. ER1A
Vibrio sp. HENC-03
Vibrio sp. Isolate30
Vibrio sp. La 4.2.2
Vibrio sp. LaRot3
Vibrio sp. MEBiC08052
Vibrio sp. McD22-P3
Vibrio sp. SCSIO 43137
Vibrio sp. THAF190c
Vibrio sp. kj40-1
Vibrio spartinae
Vibrio taketomensis
Vibrio thalassae
Vibrio tubiashii
Vibrio vulnificus
Virgisporangium aurantiacum
Vitreimonas flagellata
Vitreoscilla filiformis
Vulcanibacillus modesticaldus
Waltera intestinalis
Wenzhouxiangella sp. XN79A
Williamsia limnetica
Williamsia soli
Williamsia sp. 1135
Williamsia sp. 1138
Williamwhitmania taraxaci
Xanthobacter dioxanivorans
Xanthomonas citri
Xinfangfangia humi
Xylanivirga thermophila
Xylophilus rhododendri
Xylophilus sp. ASV27
Yoonia vestfoldensis
Zavarzinia aquatilis
Zhihengliuella halotolerans
Zoogloea dura
Zoogloea oryzae
Zoogloea ramigera
Zoogloea sp. 1C4
Zoogloea sp. LCSB751
Zooshikella ganghwensis
Clostridium nexile
Eubacterium tenue
Halarcobacter arenosus
Phormidium sp. ETS-05
Ruminococcus gnavus
Ruminococcus torques
Clostridium nexile
Eubacterium tenue
Halarcobacter arenosus
Phormidium sp. ETS-05
Ruminococcus gnavus
Ruminococcus torques
acyl-carrier protein
acyl-carrier protein reductase Haliea sp. ETY-M
acyl-carrier-protein dehydratase FabA Methyloceanibacter methanicus
acyl-carrier-protein dehydratase FabA Methyloceanibacter stevinii
acyl-carrier-protein dehydratase FabA Methyloceanibacter superfactus
acyl-carrier-protein dehydratase FabZ Methyloceanibacter marginalis
acyl-carrier-protein dehydratase FabZ Methyloceanibacter methanicus
acyl-carrier-protein dehydratase FabZ Methyloceanibacter stevinii
acyl-carrier-protein dehydratase FabZ Methyloceanibacter superfactus
acyl-carrier-protein synthase II Methyloceanibacter methanicus
acyl-carrier-protein synthase II Methyloceanibacter stevinii
acyl-carrier-protein synthase II Methyloceanibacter superfactus
acyl-carrier-protein--UDP-N-acetylglucosamine O-acyltransferase
Methyloceanibacter marginalis
acyl-carrier-protein--UDP-N-acetylglucosamine O-acyltransferase
Methyloceanibacter methanicus
acyl-carrier-protein--UDP-N-acetylglucosamine O-acyltransferase
Methyloceanibacter stevinii
acyl-carrier-protein--UDP-N-acetylglucosamine O-acyltransferase
Methyloceanibacter superfactus
bacteria endosymbiont of Bathymodiolus brooksi
bacteria endosymbiont of Bathymodiolus childressi
bacteria endosymbiont of Bathymodiolus heckerae
bacterial endosymbiont of Idas sp.
bacterium BMS3Abin04
bacterium HR30
bacterium M00.F.Ca.ET.159.01.1.1
bacterium enrichment culture clone K11-1
bacterium enrichment culture clone K14
bacterium enrichment culture clone K7-3 + K8 + K12
bacterium enrichment culture clone K8-1
bacterium symbiont of Siboglinum cf. poseidoni
bacterium
biofilm metagenome
candidate division NC10 bacterium
candidate division SRI bacterium Aalborg_AAW-1
candidate division WWE3 bacterium GW2011_GWA1_41_8
candidate division WWE3 bacterium GW2011_GWC1_41_7
candidate division Zixibacteria bacterium
endosymbiont of Bathymodiolus azoricus
endosymbiont of Bathymodiolus childressi sp. M34.1
endosymbiont of Bathymodiolus childressi sp. M34.38
endosymbiont of Bathymodiolus childressi sp. M34.3
endosymbiont of Bathymodiolus puteoserpentis
endosymbiont of Lamellibrachia barhami
endosymbiont of Ridgeia piscesae
endosymbiont of Tevnia jerichonana
marine metagenome
methane-oxidizing endosymbiont of Gigantopelta aegis
methanotroph ‘isolate 1’
methanotroph ‘isolate 2’
methanotroph B8
methanotroph C9
methanotroph FL-DIKO
methanotroph FL20
methanotroph H8
methanotroph K2-10
methanotroph K2-14
methanotroph K3-16
methanotroph K3-21
methanotroph M11
methanotroph M13
methanotroph M5
methanotroph M8
methanotroph RSKB-FBF11
methanotroph RSKB-FBF1
methanotroph RSKB-FBF5
methanotroph RSKB-FBF6
methanotroph RSKB-FBF8
methanotroph WI-11
methanotroph WI-13
methanotroph WI-14
methanotroph WI-15
methanotrophic alpha proteobacterium 2-8e
methanotrophic alpha proteobacterium RG
methanotrophic alpha proteobacterium Wd-C12
methanotrophic alpha proteobacterium arc2
methanotrophic alpha proteobacterium sakb1
methanotrophic bacterial endosymbiont of Bathymodiolus sp.
methanotrophic bacterium LK5
methanotrophic bacterium Y
methanotrophic endosymbiont of Bathymodiolus azoricus (Menez Gwen)
methanotrophic endosymbiont of Bathymodiolus japonicus
methanotrophic endosymbiont of Bathymodiolus platifrons
methanotrophic endosymbiont of Bathymodiolus puteoserpentis
(Logatchev)
mine drainage metagenome
similarity to soluble methane monooxygenase hydroxylase component
alpha-subunit methanotrophic bacterial endosymbiont of Bathymodiolus
sp.
sulfur-oxidizing endosymbiont of Gigantopelta aegis
thermophilic methanotroph HB
type I methanotrophic bacterium
type II methanotroph AML-A3
type II methanotroph AML-A6
type II methanotroph CSC-1
type II methanotrophic bacterium
unclassified Aeromonas
unclassified Alkalimarinus
unclassified Amycolatopsis
unclassified Anaeromyxobacter
unclassified Anaerotruncus
unclassified Azospirillum
unclassified Bradyrhizobium
unclassified Burkholderia
unclassified Caballeronia
unclassified Caldicellulosiruptor
unclassified Campylobacter
unclassified Candidatus Frackibacter
unclassified Clostridium
unclassified Colwellia
unclassified Crossiella
unclassified Desulfovibrio
unclassified Fusibacter
unclassified Geothrix
unclassified Haematospirillum
unclassified Haloarcula
unclassified Halomicroarcula
unclassified Helicobacter
unclassified Kribbella
unclassified Leptolyngbya
unclassified Leptospira
unclassified Limnothrix
unclassified Magnetospirillum
unclassified Methylacidiphilum
unclassified Methylocaldum
unclassified Methylocystis
unclassified Methylomonas
unclassified Methylosinus
unclassified Moritella
unclassified Mycobacterium
unclassified Mycolicibacter
unclassified Nitrosomonas
unclassified Nitrosospira
unclassified Nocardioides
unclassified Oceanispirochaeta
unclassified Poseidonibacter
unclassified Pseudodesulfovibrio
unclassified Pseudonocardia
unclassified Rhodococcus
unclassified Roseburia
unclassified Salinivibrio
unclassified Simplicispira
unclassified Streptomyces
unclassified Sulfolobus
unclassified Synechocystis
unclassified Thauera
unclassified Thioalkalivibrio
unclassified Treponema
unclassified Undibacterium
unclassified Wenzhouxiangella
uncultured Alphaproteobacteria bacterium
uncultured Candidatus Methanogranum sp.
uncultured Candidatus Methylomirabilis sp.
uncultured Gammaproteobacteria bacterium
uncultured Methylobacter sp.
uncultured Methylocaldum sp.
uncultured Methyloceanibacter sp.
uncultured Methylocella sp.
uncultured Methylococcaceae bacterium
uncultured Methylococcales bacterium
uncultured Methylococcus sp.
uncultured Methylocystaceae bacterium
uncultured Methylocystis sp. GSC357
uncultured Methylocystis sp.
uncultured Methylomicrobium sp.
uncultured Methylomonas sp.
uncultured Methyloprofundus sp.
uncultured Methylosarcina sp.
uncultured Methylosinus sp.
uncultured Nitrosospira sp.
uncultured Rubrobacteraceae bacterium
uncultured Solirubrobacteraceae bacterium
uncultured ammonia-oxidizing bacterium
uncultured ammonia-oxidizing beta proteobacterium
uncultured archaeon
uncultured bacterium Bakchar31
uncultured bacterium Bakchar7
uncultured bacterium FW-18
uncultured bacterium FW-1
uncultured bacterium FW-36
uncultured bacterium FW-47
uncultured bacterium FW-50
uncultured bacterium M84-P36
uncultured bacterium M84-P3
uncultured bacterium MR1
uncultured bacterium MR2
uncultured bacterium MR4
uncultured bacterium MR5
uncultured bacterium PS-45
uncultured bacterium PS-49
uncultured bacterium PS-80
uncultured bacterium UPO45
uncultured bacterium WC301-37
uncultured bacterium WC306-17
uncultured bacterium WC306-5
uncultured bacterium gp12
uncultured bacterium gp13
uncultured bacterium gp3
uncultured bacterium
uncultured beta proteobacterium
uncultured candidate division NC10 bacterium
uncultured eubacterium pAMC501
uncultured eubacterium pAMC503
uncultured eubacterium pAMC507
uncultured eubacterium pAMC507s
uncultured eubacterium pAMC509
uncultured eubacterium pAMC510
uncultured eubacterium pAMC511
uncultured eubacterium pAMC512
uncultured eubacterium pAMC514
uncultured eubacterium pAMC519
uncultured eubacterium pAMC521
uncultured eubacterium pAMC523
uncultured eubacterium pAMC524
uncultured eubacterium pAMC526
uncultured eubacterium pAMC528
uncultured groundwater bacterium 1
uncultured groundwater bacterium 2
uncultured methane-oxidizing alpha proteobacterium
uncultured methane-oxidizing bacterium
uncultured methane-oxidizing gamma proteobacterium
uncultured methane-oxidizing proteobacterium
uncultured methanotroph ‘Maine 6’
uncultured methanotroph ‘Maine 7’
uncultured methanotroph ‘Maine 8’
uncultured methanotroph ‘Maine 9’
uncultured methanotroph ‘Rold 1’
uncultured methanotroph ‘Rold 2’
uncultured methanotroph ‘Rold 3’
uncultured methanotroph ‘Rold 4’
uncultured methanotroph ‘Rold 5’
uncultured methanotroph ‘type 1’
uncultured methanotroph ‘type 10’
uncultured methanotroph ‘type 11’
uncultured methanotroph ‘type 12’
uncultured methanotroph ‘type 13’
uncultured methanotroph ‘type 2’
uncultured methanotroph ‘type 3’
uncultured methanotroph ‘type 4’
uncultured methanotroph ‘type 5’
uncultured methanotroph ‘type 6’
uncultured methanotroph ‘type 7’
uncultured methanotroph ‘type 8’
uncultured methanotroph ‘type 9’
uncultured methanotroph RA14
uncultured methanotroph RA21
uncultured methanotrophic alpha proteobacterium
uncultured methanotrophic bacterium M84-P38
uncultured methanotrophic bacterium M84-S21
uncultured methanotrophic bacterium M84-S28
uncultured methanotrophic bacterium M84-S36
uncultured methanotrophic bacterium M84-S38
uncultured methanotrophic bacterium M84-S3
uncultured methanotrophic bacterium M84-S42
uncultured methanotrophic bacterium M84-S49
uncultured methanotrophic bacterium M84-S6
uncultured methanotrophic bacterium M84-S7
uncultured methanotrophic bacterium M90-P36
uncultured methanotrophic bacterium
uncultured methanotrophic gamma proteobacterium
uncultured methanotrophic proteobacterium
uncultured microorganism
uncultured peat bog bacterium 24
uncultured prokaryote
uncultured proteobacterium pmoA-MO2
uncultured proteobacterium pmoA-MO3
uncultured proteobacterium
uncultured putative methanotroph pmoA-RC1
uncultured putative methanotroph pmoA-RC2
uncultured putative methanotroph pmoA-RC4
uncultured putative methanotroph
uncultured soil bacterium
uncultured type I methanotroph
uncultured type II methanotroph

TABLE 1B
Genera of methanotrophs and/or nitrogen fixing bacteria

Genus
Sphingopyxis
Methylomonas
Pseudoxanthomonas
Shinella
Devosia
Planobacterium
Ochrobactrum
Methylosinus
Hyphomicrobium
Genus
Bosea
Kaistia
Ancylobacter
Achromobacter
Allorhizobium
Mesorhizobium
Hydrogenophaga
Acidovorax
Agrobacterium
Genus
Reyranella
Enterococcus
Flavobacterium
Kaistella
Methylococcus
Stenotrophomonas
Variovorax

TABLE 1C
Species of methanotrophs and/or nitrogen fixing bacteria

Species
Achromobacter spanius
Acidovorax sp
Agrobacterium pusense
Allorhizobium sp
Ancylobacter rudongensis
Bosea robiniae
Chryseobacterium faecale
Chryseobacterium sp
Devosia sp
Enterococcus gallinarum
Flavobacteriales bacterium UBA6726

Species
Flavobacteriia bacterium
Flavobacterium lindanitolerans
Hydrogenophaga sp
Hyphomicrobium zavarzinii
Kaistia sp
Marnyiella aurantia
Marnyiella aurantia
Mesorhizobium australicum
Methylococcus capsulatus
Methylomonas methanica
Methylomonas sp
Species
Methylosinus trichosporium
Ochrobactrum teleogrylli
Pseudoxanthomonas jiangsuensis
Pseudoxanthomonas mexicana
Reyranella sp
Shinella granuli
Sphingopyxis sp
Sphingopyxis terrae
Stenotrophomonas maltophilia
Variovorax sp

TABLE 1D
Strains of methanotrophs and/or nitrogen fixing bacteria

| Accession number | Strain |
|---|---|
| GCF_019078745.1 | Sphingopyxis terrae subsp. terrae YC-JH3 |
| GCF_001644115.1 | Methylomonas methanica R-45371 |
| GCF_014211895.1 | Pseudoxanthomonas mexicana GTZY |
| GCF_001644035.1 | Methylomonas methanica R-45363 |
| GCF_010093385.1 | Pseudoxanthomonas mexicana DSM 17121 |
| GCF_002277025.1 | Sphingopyxis sp. GW247-27LB |

| Accession number | Strain |
|---|---|
| GCF_000371385.1 | Sphingopyxis sp. MC1 |
| GCF_014397415.1 | Pseudoxanthomonas mexicana GTZY2 |
| GCF_001610975.1 | Sphingopyxis terrae subsp. terrae NBRC |
| | 15098 203-1 |
| GCF_000365425.1 | Methylomonas sp. MK1 |
| GCF_001652565.1 | Shinella sp. HZN7 |
| GCF_000515215.1 | Methylomonas sp. 11b |
| GCF_001468495.1 | Sphingopyxis sp. A083 |

| Accession number | Strain |
|---|---|
| GCF_017815095.1 | Marnyiella aurantia cx-311 |
| GCF_002752655.1 | Methylosinus trichosporium OB3b |
| GCF_013087535.1 | Ochrobactrum soli MTP-C0764 |
| GCF_004341885.1 | Shinella granuli DSM 18401 |
| GCF_010093185.1 | Pseudoxanthomonas jiangsuensis DSM 22398 |
| GCF_000383415.1 | Hyphomicrobium zavarzinii ATCC 27496 ZV-622 |
| GCF_008831505.1 | Chryseobacterium sp. SNU WT7 |

| Accession number | Strain |
|---|---|
| GCF_900243435.1 | Ochrobactrum haematophilum FI11154 |
| GCF_900177325.1 | Mesorhizobium australicum B5P |
| GCF_003667905.1 | Agrobacterium tumefaciens 12D1 |
| GCF_006714945.1 | Bosea sp. AK1 |
| GCF_003634645.1 | Acidovorax sp. 94 |
| GCF_006376685.1 | [Ochrobactrum] teleogrylli LCB8 |
| GCA_003963445.1 | Ancylobacter aquaticus UV5 |
| GCF_012272675.1 | Agrobacterium pusense FDAARGOS 633 |
| GCF_019218805.1 | Acidovorax sp. sif0632 |
| GCF_003664555.1 | Ochrobactrum soli BO-7 |
| Accession number | Strain |
| GCA_014190925.1 | Rhizobium sp. RAS22 |
| GCF_000008325.1 | Methylococcus capsulatus Bath |
| GCF_019195395.1 | Chryseobacterium faecale F4 |
| GCF_001426505.1 | Variovorax sp. Root473 |
| GCF_003053405.1 | Agrobacterium pusense KCJK7997 |
| GCF_001189595.1 | Achromobacter spanius CGMCC9173 |
| GCF_001411535.1 | Acidovorax sp. SD340 |
| GCF_014138475.1 | Agrobacterium sp. RC10-4-1 |
| GCF_002846575.1 | Flavobacterium lindanitolerans IP-10 |
| GCF_003633835.1 | Acidovorax sp. 93 |
| Accession number | Strain |
| GCF_003031575.1 | Agrobacterium pusense S2 |
| GCA_002799235.1 | Stenotrophomonas maltophilia E729 |
| GCF_003994415.1 | Achromobacter spanius strain UQ283 |
| GCA_017357485.1 | Rhizobium sp. ZX09 |
| GCF_001429415.1 | Bosea sp. Root670 |
| GCF_013267155.1 | Agrobacterium pusense FDAARGOS 618 |
| GCF_000304595.1 | Bradyrhizobium lupini HPC(L) |
| GCF_900102525.1 | Bosea robiniae DSM 26672 |
| GCF_013376875.1 | Acidovorax sp. JMULE5 |

In certain embodiments, a species that is capable of consuming methane comprises a methanotroph or a methanophile. In some embodiments, a methanotroph or methanophile may express methane monooxygenase or methyl coenzyme A reductase (MCAR). In some embodiments, a methanotroph or methanophile may express other enzymes that aide in the oxidation of methane or consumption of methane.

In some embodiments, an archaea can consume methane. In certain embodiments, the consortium of bacteria may further comprise an archaea. In certain embodiments, the consortium of bacteria further comprises an archaea that consumes methane. In certain embodiments, a bacterial or archaeal species that is capable of consuming methane expresses MMO. In certain embodiments, a bacterial or archaeal species that is capable of consuming methane expresses MCAR.

In some embodiments, one or more bacterial species of the bacterial consortium may be capable of consuming methane. In some cases, one or more species of the bacterial consortia may facilitate or enhance the ability of other bacteria in the consortium to consume methane. In some examples, a bacterial strain may be able to consume methane when in combination with a different bacterial strain, or in a certain bacterial consortium, but may be unable to consume methane in a monoculture. In some examples, a bacterial strain may not be able to consume methane when in combination with a different bacterial strain, or in a certain bacterial consortium, but may be unable to consume methane in a monoculture.

### Growth and Survival

The methane-consuming organisms described herein may use a carbon source and a source of oxygen to grow and live. Carbon sources include without limitation methane, methanol, formate, methylamine, trimethylamine, and mixtures thereof. Methane can be obtained from a natural or an industrial source. In some embodiments, nonlimiting examples of natural sources of methane may include biogas, agriculturally-generated methane, dairy farms, ambient air, compost gas, livestock emissions, crop gas, or any combination thereof. In some embodiments, the industrial source of methane may be selected from the group consisting of, but not limited to, natural gas, synthetic natural gas, natural gas hydrates, stranded natural gas, shale gas, flared gas, coal mine methane, coal bed methane, methane produced from catalytic cracking of olefins or organic matter, landfill gas, associated petroleum gas, oil refineries, municipal compost, industrial compost, agricultural harvesting or processing, food processing, shipping, or methane produced as an unwanted by product from CO hydrogenation and hydrogenolysis reactions. Oxygen sources may include but are not limited to air, enriched air, 02 from fractional distillation, pressure swing adsorption, an oxygen concentrator, or electrolysis of water and liquid 02.

In some embodiments, the carbon source may comprise CH₄ at atmospheric concentrations. In some embodiments, the carbon source may comprise CH₄ at about 1.4, 1.5, 1.6, 1.7, 1.8, or 1.9 parts per million (ppm). In some embodiments, the carbon source may comprise CH₄ at a concentration of at least about 1.0, 1.1, 1.2, 1.3, 1.4, 1.5, 1.6, 1.7, 1.8, 1.9, 2.0, 2.1, 2.2, 2.3, 2.4, or 2.5 ppm. In some embodiments, the carbon source may comprise CH₄ at a concentration of more than about 1.0, 1.1, 1.2, 1.3, 1.4, 1.5, 1.6, 1.7, 1.8, 1.9, 2.0, 2.1, 2.2, 2.3, 2.4, or 2.5 ppm. In some embodiments, the carbon source may be enriched for CH₄. In some embodiments, the CH₄ may be present at a concentration of about 5, 10, 50, 100, 500, 1,000, 5,000, 10,000, 50,000, 100,000, or more ppm. In some embodiments, the methane source may comprise CH₄ at about 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, 30,000, 35,000, 40,000, 45,000, or 50,000 parts per million (ppm).

In some embodiments, the oxygen source may comprise 02 at atmospheric concentration. In some embodiments, the oxygen source may comprise 02 at about 206,000, 207,000, 208,000, 209,000, or 210,000 ppm. In some embodiments, the oxygen source may comprise 02 at a concentration between about 180,000 ppm and 210,000 ppm. In some embodiments, the oxygen source may comprise 02 at a concentration of at least about 180,000, 190,000, 200,000, 201,000, 202,000, 203,000, 204,000, 205,000, 206,000, 207,000, 208,000, 209,000, or 210,000 ppm.

The ratio of oxygen to carbon source can be varied in order to improve production of the desired products or to optimize the efficiency of the microbial reaction and ultimately improve production or to improve growth. In some embodiments, the ratio of 02 to CH₄ in the gaseous substrate can range from about 0.1:1 to about 15:1. In some embodiments, the ratio of 02 to CH₄ in the gaseous substrate can range from about 15:1 to about 12.5:1, about 15:1 to about 10:1, about 15:1 to about 7.5:1, about 15:1 to about 5:1, about 15:1 to about 4:1, about 15:1 to about 3:1, about 15:1 to about 2:1, about 15:1 to about 1:1, about 15:1 to about 0.5:1, about 15:1 to about 0.25:1, about 15:1 to about 0.1:1, about 12.5:1 to about 10:1, about 12.5:1 to about 7.5:1, about 12.5:1 to about 5:1, about 12.5:1 to about 4:1, about 12.5:1 to about 3:1, about 12.5:1 to about 2:1, about 12.5:1 to about 1:1, about 12.5:1 to about 0.5:1, about 12.5:1 to about 0.25:1, about 12.5:1 to about 0.1:1, about 10:1 to about 7.5:1, about 10:1 to about 5:1, about 10:1 to about 4:1, about 10:1 to about 3:1, about 10:1 to about 2:1, about 10:1 to about 1:1, about 10:1 to about 0.5:1, about 10:1 to about 0.25:1, about 10:1 to about 0.1:1, about 7.5:1 to about 5:1, about 7.5:1 to about 4:1, about 7.5:1 to about 3:1, about 7.5:1 to about 2:1, about 7.5:1 to about 1:1, about 7.5:1 to about 0.5:1, about 7.5:1 to about 0.25:1, about 7.5:1 to about 0.1:1, about 5:1 to about 4:1, about 5:1 to about 3:1, about 5:1 to about 2:1, about 5:1 to about 1:1, about 5:1 to about 0.5:1, about 5:1 to about 0.25:1, about 5:1 to about 0.1:1, about 4:1 to about 3:1, about 4:1 to about 2:1, about 4:1 to about 1:1, about 4:1 to about 0.5:1, about 4:1 to about 0.25:1, about 4:1 to about 0.1:1, about 3:1 to about 2:1, about 3:1 to about 1:1, about 3:1 to about 0.5:1, about 3:1 to about 0.25:1, about 3:1 to about 0.1:1, about 2:1 to about 1:1, about 2:1 to about 0.5:1, about 2:1 to about 0.25:1, about 2:1 to about 0.1:1, about 1:1 to about 0.5:1, about 1:1 to about 0.25:1, about 1:1 to about 0.1:1, about 0.5:1 to about 0.25:1, about 0.5:1 to about 0.1:1, or about 0.25:1 to about 0.1:1. In some embodiments, the ratio of O₂ to CH₄ in the gaseous substrate can range from about 15:1, about 12.5:1, about 10:1, about 7.5:1, about 5:1, about 4:1, about 3:1, about 2:1, about 1:1, about 0.5:1, about 0.25:1, or about 0.1:1. In some embodiments, the ratio of O₂ to CH₄ in the gaseous substrate can range from at least about 15:1, about 12.5:1, about 10:1, about 7.5:1, about 5:1, about 4:1, about 3:1, about 2:1, about 1:1, about 0.5:1, or about 0.25:1. In some embodiments, the ratio of O₂ to CH₄ in the gaseous substrate can range from at most about 12.5:1, about 10:1, about 7.5:1, about 5:1, about 4:1, about 3:1, about 2:1, about 1:1, about 0.5:1, about 0.25:1, or about 0.1:1. The reaction stoichiometry between CH₄ and O₂ may vary depending on the pathway utilized by the microorganism(s). In certain embodiments, the reaction stoichiometry between CH₄ and O₂ may change over time. In some embodiments, ambient air provides an alternative to O₂. In varied embodiments, the carbon source and oxygen source can be introduced to the consortium of bacteria as one stream or as separate streams. A non-limiting example comprises a natural gas stream that may be blended with an oxygen containing stream, e.g. air, to provide a desired O₂:CH₄ ratio.

In some embodiments, additional components may improve the survival of methanotrophs. In certain embodiments, a liquid or solid nutrient media may contain nutrients suitable to sustain viability of methanotrophs over time. Media suitable for culturing methane consuming bacteria are known in the art. For example, suitable media are described in Kaluzhnaya et al., 2001, Dedysh et al., 2002, Whittenbury et al., 1970, and Ojala et al., 2011. In particular embodiments, the media is a minimal mixture of salts. The composition may vary in salt content. Exemplary nutrient medium compositions are set forth in Table 2.

TABLE 2
Nutrient medium composition for methanotrophic bacteria

| Compound | Range (g/L) |
|---|---|
| KNO₃ | 0-1 |
| MgSO₄ | 0.04-1 |
| CaCl₂ | 0.01-0.2 |
| Na₂HPO₄ | 0.2-1 |
| KH₂PO₄ | 0.1-1 |
| NaCl | 0-10 |
| Na₂CO₃ | 0-1 |
| NaHCO₃ | 0-8.4 |
| Na₂EDTA | 0-5 |
| FeSO₄•7H₂O | 0.002-0.2 |
| ZnSO₄•7H₂O | 0.0001-0.8 |
| MnCl₂•4H₂O | 0.00003-0.04 |
| CoCl₂•6H₂O | 0.0002-0.2 |
| CuSO₄•5H2O | 0.0001-0.008 |
| NiCl₂•6H₂O | 0.00002-0.02 |
| Na₂MoO₄•2H₂O | 0.00003-0.05 |
| H₃BO₃ | 0-0.06 |

In some embodiments of the in the present disclosure, the media used to grow methane-consuming bacteria may comprise nitrate mineral salts (NMS) culture media. In further embodiments of the present disclosure, the media used to grow methane-consuming bacteria may comprise MgSO₄·7H₂O ranging from about 0.04 g/L to about 1 g/L, CaCl₂·6H₂O ranging from about 0.007 g/L to about 0.2 g/L, NaCl, KH₂PO₄, Na₂CO₃, Na₂-EDTA, FeSO₄·7H₂O, ZnSO₄·7H₂O, MnCl₂·4H₂O, H₃BO₃ ranging from about 0.02 g/L to about 0.03 g/L, CoCl₂·6H₂O ranging from about 0.02 g/L to about 0.2 g/L, CuCl₂·2H₂O, NiCl₂·6H₂O, and Na₂MoO₄ 2H₂O ranging from about 0.003 g/L to about 0.05 g/L. In some embodiments, the media used to grow methane-consuming bacteria may comprise mineral medium, basic mineral salts medium, or nitrate-free mineral salts medium.

In some embodiments of the present disclosure, the media used to grow methane-consuming bacteria comprises at least one nitrogen source such as KNO₃, NaNO₃, NH₄Cl, (NH₄)₂SO₄, urea, or mixtures thereof. In further embodiments of the present disclosure, the media used to grow methane-consuming bacteria comprises at least one nitrogen source comprising KNO₃, NaNO₃, or mixtures thereof. In some embodiments, the media used to grow methane-consuming bacteria is nitrogen-free.

In some embodiments, some nutrients being added affect the growth rate and the amount of lipids produced by the particular bacterium. In some embodiments, it can be desirable to have a concentration of Cu⁺² in the media or environment of at least 7.5 mol/gram dry weight (gDW). In some embodiments, gDW is the measure of mass of a completely dried substance. In some embodiments, the amount of copper may eventually become toxic to the bacterium. In some embodiments, a concentration of Cu⁺² in the media or environment may be within a range of about 7 μM to about 20 μM. In some embodiments, a concentration of Fe⁺² in the media or environment may be within a range of about 5 μM to about 15 μM (Table 2). In some embodiments, a concentration of NO₃⁻ in the media or environment may vary from about 5 mmol/gDW to about 8 mmol/gDW. Additionally, in some embodiments, a concentration of PO₄⁻³ in the media or environment could range from about 1.4 mmol/gDW to about 2 mmol/gDW.

In some embodiments, additives may be used to support the growth or viability of methanotrophs. In some embodiments, additives may be used to increase the methane consumption of one or more bacteria. In some embodiments, an additive may comprise copper, tungsten, iron, nickel, molybdenum, cerium, manganese, zinc, cobalt, boron, one or more lanthanides, or a combination thereof. In some embodiments, a lanthanide may comprise lanthanum, cerium, praseodymium, neodymium, promethium, samarium, europium, gadolinium, terbium, dysprosium, holmium, erbium, thulium, ytterbium, lutetium, or any combination thereof.

### Measuring CH₄

Methods for measuring CH₄ consumption are known in the art, for example, as disclosed by Myung et al. (2015. Environ Sci Tech. 49(18):10969-75), which is incorporated herein by reference in its entirety. In some embodiments, measuring CH₄ consumption may comprise measuring the concentration of CH₄ consumed by the consortium. In some embodiments, measuring CH₄ consumption may comprise measuring the concentration of CH₄ in a closed system by determining the change in concentration of CH₄ at an influx as compared to the concentration of CH₄ at an outflux. In some embodiments, measuring CH₄ consumption may comprise measuring the concentration of CH₄ in a closed system by determining the change in concentration of CH₄ at one earlier time point as compared to the concentration of CH₄ at one or more later time points. In some embodiments, measuring CH₄ concentration in air may comprise using gas chromatography. In some embodiments, measuring CH₄ concentration in the air may comprise using flame ionization detection gas chromatography. In some embodiments, CH₄ concentration is measured by detecting the ions formed by combustion of a sample in a hydrogen or air flame. In some embodiments, an instrument capable of gas chromatography can be modified to suit various environments. In certain embodiments, features such as column length of the gas chromatography instrument, analysis cycle time, sample size can be modified. In some embodiments, CH₄ consumption may be determined by a methane detector. In certain embodiments, CH₄ consumption may be determined by a portable methane detector.

In some embodiments, CH₄ consumption may be measured using a bio-assay. In certain embodiments, bacteria may be grown on or in media containing methane as the sole carbon source. In some embodiments, measuring bacteria grown on methane-containing media by methods such as weight or optical density (for example, OD₆₀₀) of bacteria suspended in culture may provide a quantitative determination of methane consumption.

In some embodiments, measuring CH₄ consumption may comprise using labeled CH₄. In some embodiments, CH₄ consumption measurements may comprise using radiolabeled elements. In some embodiments, radiolabeled elements may comprise using ³H, ¹⁴C, or ¹³C In some embodiments, the radiolabeled elements may be used to measure ³H₂O, C³H₄, ¹⁴CH₄, or ¹³CH₄, ¹³CO₂, ¹⁴CO₂. In some embodiments, bacteria can be incubated with radiolabeled ¹⁴CH₄. In some embodiments, bacteria can be incubated with radiolabeled ¹⁴CH₄ by introducing labeled ¹⁴CH₄ into the headspace of a reactor, incubator, or other closed system. In some embodiments, radiolabeled ¹⁴CH₄ may be introduced to soil samples comprising a consortium of bacteria. In some embodiments, radiolabeled ¹⁴CH₄ is measured in the consortium of bacteria. In some embodiments, radiolabeled ¹⁴CH₄ is measured in lipids or another cellular component of the consortium of bacteria. In some embodiments, measuring radiolabeled ¹⁴CH₄ may comprise using gas chromatography. In some embodiments, measuring radiolabeled ¹⁴CH₄ may comprise using flame ionization detection gas chromatography. In some embodiments, measuring CH₄ consumption may comprise using mass spectrometry. In some embodiments, determining methane consumption of a consortium of bacteria incubated with radiolabeled ¹⁴CH₄ may be compared to a similar sample that was not incubated with radiolabeled ¹⁴CH₄ or to a sample that lacks the capacity to consume CH₄. In some embodiments, measuring CH₄ consumption may comprise measuring gas using a reactor.

In some embodiments, CH₄ consumption may be measured by yield of the bacterial consortium or any species within the bacterial consortium. Yield is defined herein as the amount of cell mass produced per gram of carbon substrate metabolized. In some embodiments, CH₄ consumption may be measured by determining carbon conversion efficiency. In certain embodiments, carbon conversion efficiency is another measure of how much carbon is assimilated into cell mass. Carbon conversion efficiency is expressed in units of g/mol methane (1 g dry wt/g methane) per g/mol biomass. Carbon conversion efficiency may be calculated assuming a biomass composition of CH₂O_{0.5} N_{0.25}.

In some embodiments, the bacterial consortium disclosed herein consumes CH₄. In certain embodiments, the bacterial consortium disclosed herein consumes CH₄ at a rate of about 1 umol/gDW per day to about 9 umol/gDW per day. In certain embodiments, the bacterial consortium disclosed herein consumes CH₄ at a rate of about 1 umol/gDW per day to about 2 umol/gDW per day, about 1 umol/gDW per day to about 3 umol/gDW per day, about 1 umol/gDW per day to about 4 umol/gDW per day, about 1 umol/gDW per day to about 5 umol/gDW per day, about 1 umol/gDW per day to about 6 umol/gDW per day, about 1 umol/gDW per day to about 7 umol/gDW per day, about 1 umol/gDW per day to about 8 umol/gDW per day, about 1 umol/gDW per day to about 9 umol/gDW per day, about 2 umol/gDW per day to about 3 umol/gDW per day, about 2 umol/gDW per day to about 4 umol/gDW per day, about 2 umol/gDW per day to about 5 umol/gDW per day, about 2 umol/gDW per day to about 6 umol/gDW per day, about 2 umol/gDW per day to about 7 umol/gDW per day, about 2 umol/gDW per day to about 8 umol/gDW per day, about 2 umol/gDW per day to about 9 umol/gDW per day, about 3 umol/gDW per day to about 4 umol/gDW per day, about 3 umol/gDW per day to about 5 umol/gDW per day, about 3 umol/gDW per day to about 6 umol/gDW per day, about 3 umol/gDW per day to about 7 umol/gDW per day, about 3 umol/gDW per day to about 8 umol/gDW per day, about 3 umol/gDW per day to about 9 umol/gDW per day, about 4 umol/gDW per day to about 5 umol/gDW per day, about 4 umol/gDW per day to about 6 umol/gDW per day, about 4 umol/gDW per day to about 7 umol/gDW per day, about 4 umol/gDW per day to about 8 umol/gDW per day, about 4 umol/gDW per day to about 9 umol/gDW per day, about 5 umol/gDW per day to about 6 umol/gDW per day, about 5 umol/gDW per day to about 7 umol/gDW per day, about 5 umol/gDW per day to about 8 umol/gDW per day, about 5 umol/gDW per day to about 9 umol/gDW per day, about 6 umol/gDW per day to about 7 umol/gDW per day, about 6 umol/gDW per day to about 8 umol/gDW per day, about 6 umol/gDW per day to about 9 umol/gDW per day, about 7 umol/gDW per day to about 8 umol/gDW per day, about 7 umol/gDW per day to about 9 umol/gDW per day, or about 8 umol/gDW per day to about 9 umol/gDW per day. In certain embodiments, the bacterial consortium disclosed herein consumes CH₄ at a rate of about 1 umol/gDW per day, about 2 umol/gDW per day, about 3 umol/gDW per day, about 4 umol/gDW per day, about 5 umol/gDW per day, about 6 umol/gDW per day, about 7 umol/gDW per day, about 8 umol/gDW per day, or about 9 umol/gDW per day. In certain embodiments, the bacterial consortium disclosed herein consumes CH₄ at a rate of at least about 1 umol/gDW per day, about 2 umol/gDW per day, about 3 umol/gDW per day, about 4 umol/gDW per day, about 5 umol/gDW per day, about 6 umol/gDW per day, about 7 umol/gDW per day, or about 8 umol/gDW per day. In certain embodiments, the bacterial consortium disclosed herein consumes CH₄ at a rate of at most about 2 umol/gDW per day, about 3 umol/gDW per day, about 4 umol/gDW per day, about 5 umol/gDW per day, about 6 umol/gDW per day, about 7 umol/gDW per day, about 8 umol/gDW per day, or about 9 umol/gDW per day.

In certain embodiments, the bacterial consortium disclosed herein consumes CH₄ at a rate of about 10 umol/gDW per day to about 15.5 umol/gDW per day. In certain embodiments, the bacterial consortium disclosed herein consumes CH₄ at a rate of about 10 umol/gDW per day to about 10.5 umol/gDW per day, about 10 umol/gDW per day to about 11 umol/gDW per day, about 10 umol/gDW per day to about 11.5 umol/gDW per day, about 10 umol/gDW per day to about 12 umol/gDW per day, about 10 umol/gDW per day to about 12.5 umol/gDW per day, about 10 umol/gDW per day to about 13 umol/gDW per day, about 10 umol/gDW per day to about 13.5 umol/gDW per day, about 10 umol/gDW per day to about 14 umol/gDW per day, about 10 umol/gDW per day to about 14.5 umol/gDW per day, about 10 umol/gDW per day to about 15 umol/gDW per day, about 10 umol/gDW per day to about 15.5 umol/gDW per day, about 10.5 umol/gDW per day to about 11 umol/gDW per day, about 10.5 umol/gDW per day to about 11.5 umol/gDW per day, about 10.5 umol/gDW per day to about 12 umol/gDW per day, about 10.5 umol/gDW per day to about 12.5 umol/gDW per day, about 10.5 umol/gDW per day to about 13 umol/gDW per day, about 10.5 umol/gDW per day to about 13.5 umol/gDW per day, about 10.5 umol/gDW per day to about 14 umol/gDW per day, about 10.5 umol/gDW per day to about 14.5 umol/gDW per day, about 10.5 umol/gDW per day to about 15 umol/gDW per day, about 10.5 umol/gDW per day to about 15.5 umol/gDW per day, about 11 umol/gDW per day to about 11.5 umol/gDW per day, about 11 umol/gDW per day to about 12 umol/gDW per day, about 11 umol/gDW per day to about 12.5 umol/gDW per day, about 11 umol/gDW per day to about 13 umol/gDW per day, about 11 umol/gDW per day to about 13.5 umol/gDW per day, about 11 umol/gDW per day to about 14 umol/gDW per day, about 11 umol/gDW per day to about 14.5 umol/gDW per day, about 11 umol/gDW per day to about 15 umol/gDW per day, about 11 umol/gDW per day to about 15.5 umol/gDW per day, about 11.5 umol/gDW per day to about 12 umol/gDW per day, about 11.5 umol/gDW per day to about 12.5 umol/gDW per day, about 11.5 umol/gDW per day to about 13 umol/gDW per day, about 11.5 umol/gDW per day to about 13.5 umol/gDW per day, about 11.5 umol/gDW per day to about 14 umol/gDW per day, about 11.5 umol/gDW per day to about 14.5 umol/gDW per day, about 11.5 umol/gDW per day to about 15 umol/gDW per day, about 11.5 umol/gDW per day to about 15.5 umol/gDW per day, about 12 umol/gDW per day to about 12.5 umol/gDW per day, about 12 umol/gDW per day to about 13 umol/gDW per day, about 12 umol/gDW per day to about 13.5 umol/gDW per day, about 12 umol/gDW per day to about 14 umol/gDW per day, about 12 umol/gDW per day to about 14.5 umol/gDW per day, about 12 umol/gDW per day to about 15 umol/gDW per day, about 12 umol/gDW per day to about 15.5 umol/gDW per day, about 12.5 umol/gDW per day to about 13 umol/gDW per day, about 12.5 umol/gDW per day to about 13.5 umol/gDW per day, about 12.5 umol/gDW per day to about 14 umol/gDW per day, about 12.5 umol/gDW per day to about 14.5 umol/gDW per day, about 12.5 umol/gDW per day to about 15 umol/gDW per day, about 12.5 umol/gDW per day to about 15.5 umol/gDW per day, about 13 umol/gDW per day to about 13.5 umol/gDW per day, about 13 umol/gDW per day to about 14 umol/gDW per day, about 13 umol/gDW per day to about 14.5 umol/gDW per day, about 13 umol/gDW per day to about 15 umol/gDW per day, about 13 umol/gDW per day to about 15.5 umol/gDW per day, about 13.5 umol/gDW per day to about 14 umol/gDW per day, about 13.5 umol/gDW per day to about 14.5 umol/gDW per day, about 13.5 umol/gDW per day to about 15 umol/gDW per day, about 13.5 umol/gDW per day to about 15.5 umol/gDW per day, about 14 umol/gDW per day to about 14.5 umol/gDW per day, about 14 umol/gDW per day to about 15 umol/gDW per day, about 14 umol/gDW per day to about 15.5 umol/gDW per day, about 14.5 umol/gDW per day to about 15 umol/gDW per day, about 14.5 umol/gDW per day to about 15.5 umol/gDW per day, or about 15 umol/gDW per day to about 15.5 umol/gDW per day. In certain embodiments, the bacterial consortium disclosed herein consumes CH₄ at a rate of about 10 umol/gDW per day, about 10.5 umol/gDW per day, about 11 umol/gDW per day, about 11.5 umol/gDW per day, about 12 umol/gDW per day, about 12.5 umol/gDW per day, about 13 umol/gDW per day, about 13.5 umol/gDW per day, about 14 umol/gDW per day, about 14.5 umol/gDW per day, about 15 umol/gDW per day, or about 15.5 umol/gDW per day. In certain embodiments, the bacterial consortium disclosed herein consumes CH₄ at a rate of at least about 10 umol/gDW per day, about 10.5 umol/gDW per day, about 11 umol/gDW per day, about 11.5 umol/gDW per day, about 12 umol/gDW per day, about 12.5 umol/gDW per day, about 13 umol/gDW per day, about 13.5 umol/gDW per day, about 14 umol/gDW per day, about 14.5 umol/gDW per day, or about 15 umol/gDW per day. In certain embodiments, the bacterial consortium disclosed herein consumes CH₄ at a rate of at most about 10.5 umol/gDW per day, about 11 umol/gDW per day, about 11.5 umol/gDW per day, about 12 umol/gDW per day, about 12.5 umol/gDW per day, about 13 umol/gDW per day, about 13.5 umol/gDW per day, about 14 umol/gDW per day, about 14.5 umol/gDW per day, about 15 umol/gDW per day, or about 15.5 umol/gDW per day.

### II. Nitrogen Fixation

Nitrogen is available as N₂ in the atmosphere, however, due to the nature of the bond between the two nitrogen atoms, its reactivity is nearly zero, which means that it is not assimilable by most living organisms. Nitrogen-fixing microorganisms, such as one or more bacteria in an embodiment of the consortium of bacteria disclosed herein, may fix nitrogen through the conversion of atmospheric nitrogen into metabolizable forms that can be assimilated by living beings, such as, for example, ammonium forms, nitrites and nitrates. In some embodiments, one or more species of the bacterial consortium disclosed herein may fix nitrogen from the atmosphere and convert it into biologically available forms for use by plants or other organisms.

In certain embodiments, one or more bacteria in the bacterial consortium disclosed herein is capable of fixing N₂. In certain embodiments, a bacteria that fixes nitrogen may reduce the nitrogen. In aerobic conditions, a bacteria capable of reducing nitrogen may reduce N₂ with H₂O and Adenosine triphosphate (ATP) into NH₃ and H₂. The ammonia (NH₃) may then be incorporated into organic compounds. In some embodiments, bacteria capable of fixing nitrogen are listed in Table 3.

In some embodiments, the fixation of N₂ is catalyzed by a nitrogenase enzyme. In certain embodiments, the nitrogenase enzyme may comprise a protein complex. In some embodiments, a nitrogenase protein complex comprises two distinct components. In various embodiments, the nitrogenase comprises a dinitrogenase reductase and a dinitrogenase. In some embodiments, dinitrogenase reductase donates two high potential electrons at a time to dinitrogenase and contains an Fe-S center that holds the electrons before donation. In some embodiments, dinitrogenase then catalyzes the reduction of N₂. In certain embodiments, fixed nitrogen can be oxidized to NO₂⁻/NO₃⁻ or assimilated by organisms.

In certain embodiments, a species that is capable of fixing nitrogen comprises a diazotroph. In order to utilize elemental nitrogen (N) for chemical synthesis, life forms combine nitrogen gas (N₂) available in the atmosphere with hydrogen in a process known as nitrogen fixation. Because of the energy-intensive nature of biological nitrogen fixation, diazotrophs have evolved sophisticated and tight regulation of the nif gene cluster in response to environmental oxygen and available nitrogen. Nif genes encode enzymes involved in nitrogen fixation (such as the nitrogenase complex) and proteins that regulate nitrogen fixation. In various embodiments, diazotrophs contain at least one of the three closely related sub-types of nitrogenase: Nif, Vnf, and Anf. In some embodiments, the catalytic components of nitrogenase include two distinct proteins: dinitrogenase and dinitrogenase reductase. In some embodiments, a nitrogenase may comprise a molybdenum-dependent (Mo-dependent) nitrogenase. In certain embodiments, the nitrogenase enzyme may be encoded for by a nif gene. In some embodiments, a nif gene may comprise nifH, nifD, nifK, nifE, nifN, and nifB. In certain embodiments, a nif gene may comprise nifH, nifD, nifK, nifE, nifN, nifB, or any combination thereof. In various embodiments, the structural components of nitrogenase may be encoded by nifH, nifD, and nifK. In some embodiments, the presence of one or more nif genes may classify a species as a diazotroph or capable of fixing nitrogen. In certain embodiments, a species that is capable of fixing nitrogen expresses nitrogenase.

In certain embodiments, the nitrogenase may comprise an alternative nitrogenase. In various embodiments, alternative nitrogenases may comprise enzyme homologs that further comprise an additional subunit in the dinitrogenase component and the absence of the heteroatom molybdenum. In some embodiments, a nitrogenase may comprise a vanadium-dependent nitrogenases. In various embodiments, a vanadium-dependent nitrogenase may be encoded by vnfH, vnfD, vnfG, and vnfK. In some embodiments, the nitrogenase may comprise an iron-only nitrogenases or be devoid of molybdenum and vandium. In certain embodiments, the nitrogenase components may be encoded by anfH, anfD, anfG, and anfK. In some embodiments, the nitrogenase may be encoded by NifH (similar to VnfH and AnfH) and/or NifD (similar to VnfD and AnfD). In some embodiments, the nitrogen-fixing bacterial consortium may comprise a superoxide-dependent nitrogenase from Streptomyces thermoautotrophicus. In some embodiments, at least one species in the consortium of bacteria comprises at least one component of the nitrogen fixation or assimilation genetic regulatory network selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

In certain embodiments, a nitrogen fixing bacteria may comprise a Gram-positive bacteria. In some cases, a Gram-positive bacteria may have a Molybdenum-Iron nitrogenase system comprising: nifH, nifD, nifK, nifB, nifE, nifN, nifX, hesA, MTV, nifW, nifS, nif, and nifI2. In some cases, a Gram positive bacteria may have a vanadium nitrogenase system comprising: vnfDG, vnfK, vnfE, vnfN, vupC, vupB, vupA, vnfV, vnfR1, vnfH, vnfR2, vnfA (transcriptional regulator). In some cases, a Gram bacteria may have an iron-only nitrogenase system comprising: anfK, anfG, anfD, anfH, anfA (transcriptional regulator). In some cases, a Gram-positive bacteria may have a nitrogenase system comprising glnB, and glnK (nitrogen signaling proteins). Some examples of enzymes involved in nitrogen metabolism in Gram-positive bacteria include glnA (glutamine synthetase), gdh (glutamate dehydrogenase), bdh (3-hydroxybutyrate dehydrogenase), glutaminase, gltAB/gltB/gltS (glutamate synthase), asnA/asnB (aspartate-ammonia ligase/asparagine synthetase), and ansA/ansZ (asparaginase). Some examples of proteins involved in nitrogen transport in Gram-positive bacteria include amtB (ammonium transporter), glnK (regulator of ammonium transport), glnPHQ/glnQHMP (ATP-dependent glutamine/glutamate transporters), glnT/alsT/yrbD/ylfA (glutamine-like proton symport transporters), and gltP/gltT/yhcl/nqt (glutamate-like proton symport transporters).

In some embodiments, expression of nitrogenase may be altered in the presence of oxygen. In some embodiments, expression of nitrogenase may be inhibited or reduced in the presence of oxygen. In certain embodiments, nitrogenase is expressed in ambient oxygen levels. In various embodiments, expression of nitrogenase may be altered in the presence of ammonium (NH₄). In various embodiments, expression of nitrogenase may be inhibited or reduced in the presence of ammonium (NH₄). In some embodiments, expression of nitrogenase may be altered in the presence of nitrate. In some embodiments, expression of nitrogenase may be inhibited or reduced in the presence of nitrate. In various embodiments, expression of nitrogenase may be altered in the presence of 2-oxoglutarate. In various embodiments, expression of nitrogenase may be inhibited or reduced in the presence of 2-oxoglutarate.

In some embodiments, the consortium of bacteria comprises at least one or more species that is capable of fixing nitrogen. In some embodiments, the consortium of bacteria comprises at least one or more diazotrophs. In certain embodiments, the bacteria that are capable of fixing nitrogen are phylogenetically diverse. In some embodiments, the consortium of bacteria comprises at least 1, 2, 3, 4, or more different bacterial phyla. In certain non-limiting embodiments, a bacteria capable of fixing nitrogen may belong to, for example, the phyla Bacteroidetes, Proteobacteria, Firmicutes, Actinobacteria, Pseudomonadota, Epsilonbacteraeota, Campylobacterota, Chlorobiota, Bacillota, Chloroflexota, Cyanobacteria, or Actinobacteria. In certain embodiments, the consortium of bacteria may comprise at least 1, 2, 3, 4, 5, 6, 7, 8, 9, 10, 15, 20, 30, or more different bacterial order. Non-limiting examples of orders for which bacteria in the consortium disclosed herein may belong include Bacteroidales, Campylobacterales, Brukholderiaceae, Chlorobiales, Dehalococcoidales, Hypomicrobiales, Bacillales, Rhodospirillales, or Enterobascterales. In certain embodiments, the consortium of bacteria may comprise at least 1, 2, 3, 4, 5, 6, 7, 8, 9, 10, 15, 20, 30, 40, 50, or more different bacterial family. In certain embodiments, a bacteria capable of fixing nitrogen may belong, in a non-limiting example, to the family Arcobacteraceae, Burkholderiaceae, Chlorobiaceae, Dehalococcoidaceae, Rhizobiaceae, Nitrobacteraceae, or Bacillaceae. In certain embodiments, the consortium of bacteria may comprise at least 1, 2, 3, 4, 5, 6, 7, 8, 9, 10, 15, 20, 30, 40, 50, or more different bacterial genera. In certain non-limiting embodiments, a bacteria capable of fixing nitrogen may belong, for example, to the genera Achromobacter, Acetobacter, Alcaligenes, Athrobacter, Azopirillum, Azotobacter, Azomonas, Beijerinckia, Clostridium, Corynebacterium, Derxia, Enterobacter, Herbaspirillum, Klebsiella, Pseudomonas, Rhodospirillum, Rhodopseudomonas, Xanthobacter. Arcobacter, Burkholderia, Chlorobaculum, Rhizobium, Bradyhizobium, Rahnella, Kosakonia, or Bacillus. In some embodiments, the consortium of bacteria may comprise 1, 2, 3, 4, 5, 6, 7, 8, 9, 10, 11, 12, 13, 14, 15, 16, 17, 18, 19, 20, 25, 30, 35, 40, 45, 50, 55, 60, or more species that are capable of fixing nitrogen. In some embodiments, the consortium of bacteria may comprise 1, 2, 3, 4, 5, 6, 7, 8, 9, 10, 11, 12, 13, 14, 15, 16, 17, 18, 19, 20, 25, 30, 35, 40, 45, 50, 55, 60, or more strains that are capable of fixing nitrogen.

In some embodiments, the consortium of bacteria may comprise a species from Table 3. In some embodiments, the consortium of bacteria may comprise 1, 2, 3, 4, 5, 6, 7, 8, 9, 10, or more species from Table 3. In certain embodiments, the consortium of bacteria may comprise one or more species from Table 3 in combination with other bacteria that may consume methane only, fix nitrogen only, or do neither consume methane nor fix nitrogen. In various embodiments, the consortium of bacteria may comprise only bacteria listed in Table 3.

TABLE 3
Species containing nifH gene homologs
Species

Clostridium citroniae WAL-17108
‘Nostoc azollae’ 0708
ANME-1 cluster archaeon ex4572_4
ANME-2 cluster archaeon HR1
ANME-2 cluster archaeon
Abditibacterium utsteinense
Absicoccus porci
Absidia repens
Abyssalbus ytuae
Acanthamoeba castellanii str. Neff
Acanthaster planci
Acanthocheilonema viteae
Acaryochloris marina
Acaryochloris sp. ‘Moss Beach’
Acaryochloris sp. CCMEE 5410
Acaryochloris sp. HICR111A
Acaryochloris thomasi
Acerihabitans arboris
Acetatifactor sp. DSM 110981
Acetitomaculum ruminis DSM 5522
Acetitomaculum ruminis
Acetivibrio alkalicellulosi
Acetivibrio cellulolyticus
Acetivibrio clariflavus DSM 19732
Acetivibrio clariflavus
Acetivibrio mesophilus
Acetivibrio straminisolvens JCM 21531
Acetivibrio straminisolvens
Acetivibrio thermocellus AD2
Acetivibrio thermocellus ATCC 27405
Acetivibrio thermocellus BC1
Acetivibrio thermocellus DSM 1313
Acetivibrio thermocellus DSM 2360
Acetivibrio thermocellus YS
Acetivibrio thermocellus
Acetoanaerobium sticklandii
Acetobacter cerevisiae
Acetobacter cibinongensis
Acetobacter estunensis
Acetobacter lambici
Acetobacter lovaniensis
Acetobacter malorum
Acetobacter oeni
Acetobacter orientalis
Acetobacter pasteurianus NBRC 3222
Acetobacter pasteurianus NBRC 3280
Acetobacter pasteurianus
Acetobacter peroxydans
Acetobacter persici
Acetobacter sp. CAG: 977
Acetobacter sp. P1H12_c
Acetobacter sp. P5B1
Acetobacterium bakii
Acetobacterium dehalogenans
Acetobacterium fimetarium
Acetobacterium paludosum
Acetobacterium sp. KB-1
Acetobacterium sp. MES1
Acetobacterium tundrae
Acetobacterium wieringae
Acetobacterium woodii DSM 1030
Acetobacterium woodii
Acetobacterium
Acetohalobium arabaticum DSM 5501
Acetohalobium arabaticum
Acetomicrobium hydrogeniformans ATCC BAA-1850
Acetomicrobium hydrogeniformans
Acetomicrobium mobile DSM 13181
Acetomicrobium mobile
Acetonema longum DSM 6540
Acetonema longum
Acetothermia bacterium 64_32
Acholeplasma laidlawii
Acholeplasma oculi
Acholeplasma sp.
Achromatium sp. WMS3
Achromobacter aestuarii
Achromobacter animicus
Achromobacter arsenitoxydans
Achromobacter deleyi
Achromobacter denitrificans
Achromobacter dolens
Achromobacter insolitus
Achromobacter insuavis
Achromobacter kerstersii
Achromobacter marplatensis
Achromobacter mucicolens
Achromobacter pestifer
Achromobacter piechaudii
Achromobacter pulmonis
Achromobacter ruhlandii
Achromobacter sp. 2789STDY5608621
Achromobacter sp. ACM05
Achromobacter sp. ACRQX
Achromobacter sp. ASV32
Achromobacter sp. B7
Achromobacter sp. DH1f
Achromobacter sp. DMS1
Achromobacter sp. ES-001
Achromobacter sp. GG226
Achromobacter sp. GbtcB20
Achromobacter sp. JUb104
Achromobacter sp. LC458
Achromobacter sp. MFA1 R4
Achromobacter sp. NFACC18-2
Achromobacter sp. Root565
Achromobacter sp. Root83
Achromobacter sp. SLBN-14
Achromobacter sp. UMC46
Achromobacter sp. UMC71
Achromobacter spanius
Achromobacter xylosoxidans
Achromobacter
Acidaminobacter sp. JC074
Acidianus brierleyi
Acidianus sulfidivorans JP7
Acidianus sulfidivorans
Acidibrevibacterium fodinaquatile
Acidicapsa ligni
Acididesulfobacillus acetoxydans
Acidiferrobacter sp. SPIII_3
Acidiferrobacter thiooxydans
Acidihalobacter aeolianus
Acidihalobacter prosperus
Acidimicrobiaceae bacterium
Acidimicrobiales bacterium
Acidiphilium multivorum AIU301
Acidiphilium multivorum
Acidiphilium rubrum
Acidiphilium sp. 21-60-14
Acidiphilium sp. 21-68-69
Acidiphilium sp. 34-60-192
Acidiphilium sp. 34-64-41
Acidiphilium sp. 37-60-79
Acidiphilium sp. 37-64-53
Acidiphilium sp. 37-67-22
Acidiphilium sp. PA
Acidiphilium sp. PM
Acidiphilium
Acidipila rosea
Acidipila sp. EB88
Acidisarcina polymorpha
Acidisoma silvae
Acidisoma sp. L85
Acidisoma sp. S159
Acidisphaera sp. L21
Acidisphaera sp. S103
Acidithiobacillales bacterium SM23_46
Acidithiobacillus ferrianus
Acidithiobacillus ferridurans
Acidithiobacillus ferriphilus
Acidithiobacillus ferrivorans SS3
Acidithiobacillus ferrivorans
Acidithiobacillus ferrooxidans ATCC 23270
Acidithiobacillus ferrooxidans ATCC 53993
Acidithiobacillus ferrooxidans
Acidithiobacillus sp. ‘AMD consortium’
Acidithiobacillus sp. GGI-221
Acidithiobacillus sp. SH
Acidithiobacillus sulfuriphilus
Acidithiobacillus thiooxidans
Acidithiobacillus
Acidobacteriota bacterium
Acidobacterium capsulatum
Acidocella aminolytica
Acidocella sp. 20-57-95
Acidocella sp. 20-58-15
Acidocella sp. 20-61-6
Acidocella sp. 20-63-7
Acidocella sp. 21-58-7
Acidocella sp. 35-58-6
Acidovorax avenae subsp. avenae ATCC 19860
Acidovorax avenae
Acidovorax caeni
Acidovorax cattleyae
Acidovorax cavernicola
Acidovorax citrulli
Acidovorax delafieldii 2AN
Acidovorax delafieldii
Acidovorax facilis
Acidovorax konjaci
Acidovorax oryzae
Acidovorax soli
Acidovorax sp. 16-64-162
Acidovorax sp. CF316
Acidovorax sp. D2M1
Acidovorax sp. GBBC 1281
Acidovorax sp. GBBC 3334
Acidovorax sp. HDW3
Acidovorax sp. JHL-3
Acidovorax sp. JS42
Acidovorax sp. KKS102
Acidovorax sp. Leaf160
Acidovorax sp. Leaf191
Acidovorax sp. Leaf76
Acidovorax sp. Leaf78
Acidovorax sp. Leaf84
Acidovorax sp. MR-S7
Acidovorax sp. NCPPB 3576
Acidovorax sp. NCPPB 4044
Acidovorax sp. Root217
Acidovorax sp. Root219
Acidovorax sp. Root267
Acidovorax sp. Root275
Acidovorax sp. Root568
Acidovorax sp. Root70
Acidovorax sp. SD340
Acidovorax sp. sic0104
Acidovorax temperans
Acidovorax valerianellae
Acidovorax wautersii
Acidovorax
Acinetobacter baumannii MDR-TJ
Acinetobacter baumannii
Acinetobacter baylyi
Acinetobacter calcoaceticus ANC 3680
Acinetobacter calcoaceticus DSM 30006 = CIP 81.8
Acinetobacter calcoaceticus NIPH 13
Acinetobacter calcoaceticus RUH 2202
Acinetobacter calcoaceticus
Acinetobacter haemolyticus
Acinetobacter nosocomialis
Acinetobacter pittii
Acinetobacter sp. 528
Acinetobacter sp. ANC 3789
Acinetobacter sp. CAG: 196
Acinetobacter sp. CCBAU 10931
Acinetobacter sp. KAM403
Acinetobacter sp. Leaf130
Acinetobacter sp. P7-22
Acinetobacter sp. P7-26
Acinetobacter sp. P7-28
Acinetobacter sp. P7-29
Acinetobacter sp. P7-6
Acinetobacter sp. P8-1
Acinetobacter sp. P8-9
Acinetobacter sp. RIT592
Acinetobacter sp. V2
Acinetobacter sp. YH12054
Acinetobacter sp. yr461
Acinetobacter
Acrocarpospora catenulata
Acrocarpospora corrugata
Acrocarpospora macrocephala
Acrocarpospora phusangensis
Acrocarpospora pleiomorpha
Actibacterium sp. 188UL27-1
Actibacterium sp. EMB200-NS6
Actibacterium ureilyticum
Actibacterium
Actinacidiphila acididurans
Actinacidiphila alni
Actinacidiphila bryophytorum
Actinacidiphila epipremni
Actinacidiphila glaucinigra
Actinacidiphila guanduensis
Actinacidiphila oryziradicis
Actinacidiphila paucisporea
Actinacidiphila rubida
Actinacidiphila soli
Actinacidiphila yeochonensis
Actinoallomurus bryophytorum
Actinoallomurus purpureus
Actinoallomurus sp. WRP6H-15
Actinoallomurus sp. WRP9H-5
Actinoalloteichus sp. AHMU CJ021
Actinobacteria bacterium 66 15
Actinobacteria bacterium HGW-Actinobacteria-10
Actinobacteria bacterium HGW-Actinobacteria-1
Actinobacteria bacterium HGW-Actinobacteria-2
Actinobacteria bacterium HGW-Actinobacteria-5
Actinobacteria bacterium OV450
Actinocatenispora rupis
Actinocatenispora sera
Actinocorallia herbida
Actinokineospora alba
Actinokineospora auranticolor
Actinokineospora bangkokensis
Actinokine ospora inagensis
Actinokineospora iranica
Actinokineospora sp. PR83
Actinokineospora xionganensis
Actinomadura alba
Actinomadura bangladeshensis
Actinomadura chibensis
Actinomadura coerulea
Actinomadura craniellae
Actinomadura fibrosa
Actinomadura flavalba
Actinomadura harenae
Actinomadura macra
Actinomadura madurae
Actinomadura mexicana
Actinomadura parvosata subsp. kistnae
Actinomadura parvosata
Actinomadura rubrisoli
Actinomadura rubrobrunea
Actinomadura rubteroloni
Actinomadura sp. 6K520
Actinomadura sp. 7K534
Actinomadura sp. CNU-125
Actinomadura sp. GC306
Actinomadura sp. KC216
Actinomadura sp. KC345
Actinomadura spongiicola
Actinomadura
Actinomyces dentalis
Actinomyces denticolens
Actinomyces gerencseriae
Actinomyces graevenitzii
Actinomyces howellii
Actinomyces massiliensis F0489
Actinomyces massiliensis
Actinomyces naeslundii
Actinomyces polynesiensis
Actinomyces provencensis
Actinomyces sp. ICM47
Actinomyces sp. S4-C9
Actinomyces sp. oral taxon 171
Actinomyces sp. oral taxon 172 str. F0311
Actinomyces sp. oral taxon 178 str. F0338
Actinomyces sp. oral taxon 877 str. F0543
Actinomyces sp. oral taxon 897
Actinomyces trachealis
Actinomyces urinae
Actinomyces
Actinomycetales bacterium JB111
Actinomycetospora callitridis
Actinomycetospora chiangmaiensis
Actinomycetospora chibensis
Actinomycetospora cinnamomea
Actinomycetospora corticicola
Actinomycetospora endophytica
Actinomycetospora lutea
Actinomycetospora soli
Actinomycetospora sp. DW7H6
Actinomycetospora sp. TBRC 11914
Actinomycetospora straminea
Actinomycetospora succinea
Actinomycetota bacterium
Actinophytocola oryzae
Actinoplanes abujensis
Actinoplanes aksuensis
Actinoplanes atraurantiacus
Actinoplanes auranticolor
Actinoplanes awajinensis
Actinoplanes bogorensis
Actinoplanes brasiliensis
Actinoplanes campanulatus
Actinoplanes cyaneus
Actinoplanes deccanensis
Actinoplanes digitatis
Actinoplanes friuliensis
Actinoplanes globisporus
Actinoplanes hotanensis
Actinoplanes hulinensis
Actinoplanes lobatus
Actinoplanes missouriensis
Actinoplanes nipponensis
Actinoplanes philippinensis
Actinoplanes polyasparticus
Actinoplanes rectilineatus
Actinoplanes regularis
Actinoplanes siamensis
Actinoplanes sp. ATCC 53533
Actinoplanes sp. DH11
Actinoplanes sp. KI2
Actinoplanes sp. L3-i22
Actinoplanes sp. LAM7112
Actinoplanes sp. M2I2
Actinoplanes sp. M4I6
Actinoplanes sp. N902-109
Actinoplanes sp. OR16
Actinoplanes sp. Pm04-4
Actinoplanes sp. SE50/110
Actinoplanes sp. TBRC 11911
Actinoplanes sp. TFC3
Actinoplanes subtropicus
Actinoplanes teichomyceticus
Actinoplanes utahensis
Actinoplanes xinjiangensis
Actinopolymorpha cephalotaxi
Actinopolymorpha pittospori
Actinopolymorpha rutila
Actinopolyspora alba
Actinopolyspora biskrensis
Actinopolyspora erythraea
Actinopolyspora halophila
Actinopolyspora righensis
Actinopolyspora saharensis
Actinosynnema mirum
Actinosynnema pretiosum
Actinosynnema sp. ALI-1.44
Actinosynnema
Actinotalea sp. K2
Actinotalea sp. M2MS4P-6
Actinotalea sp. Marseille-Q4924
Actinotalea subterranea
Acutalibacter sp. 1XD8-33
Acuticoccus kalidii
Acuticoccus kandeliae
Acuticoccus sediminis
Adhaeribacter aerolatus
Adhaeribacter aquaticus
Adhaeribacter arboris
Adhaeribacter pallidiroseus
Adhaeribacter radiodurans
Adhaeribacter rhizoryzae
Adhaeribacter swui
Adlercreutzia sp. ZJ242
Adlercreutzia sp. ZJ473
Adonisia turfae
Advenella kashmirensis
Aegicerativicinus sediminis
Aeromicrobium camelliae
Aeromicrobium chenweiae
Aeromicrobium choanae
Aeromicrobium endophyticum
Aeromicrobium fastidiosum
Aeromicrobium ginsengisoli
Aeromicrobium phragmitis
Aeromicrobium sp. 9AM
Aeromicrobium sp. A1-2
Aeromicrobium sp. CCBAU 10887
Aeromicrobium sp. CFBP 8757
Aeromicrobium sp. HA
Aeromicrobium sp. Leaf245
Aeromicrobium sp. Leaf289
Aeromicrobium sp. Leaf350
Aeromicrobium sp. PE09-221
Aeromicrobium sp. Root236
Aeromicrobium sp. Root344
Aeromicrobium sp. zg-Y869
Aeromicrobium stalagmiti
Aeromicrobium tamlense
Aeromicrobium
Aeromonas allosaccharophila
Aeromonas caviae
Aeromonas dhakensis
Aeromonas hydrophila
Aeromonas media
Aeromonas piscicola
Aeromonas salmonicida subsp. salmonicida A449
Aeromonas salmonicida
Aeromonas schubertii
Aeromonas simiae
Aeromonas sp. FDAARGOS 1405
Aeromonas sp. FDAARGOS 1407
Aeromonas sp. IPPW-29
Aeromonas sp. IPPW-33a
Aeromonas sp. JL9
Aeromonas sp. L_1B5_3
Aeromonas sp. P3-26
Aeromonas sp. QDB51
Aeromonas sp. QDB62
Aeromonas sp. ZOR0002
Aeromonas sp. gx-126
Aeromonas taiwanensis
Aeromonas veronii AER39
Aeromonas veronii AMC34
Aeromonas veronii
Aeromonas
Aestuariibacter salexigens
Aestuariibacter sp. GS-14
Aestuariirhabdus litorea
Aestuariispira insulae
Aestuariivirga litoralis
Aestuariivita boseongensis
Aestuariivivens marinum
Aestuariivivens sp. NBU2969
Aethina tumida
Aetokthonos hydrillicola
Affinibrenneria salicis
Afifella marina DSM 2698
Afifella pfennigii
Afifella sp. H1R
Afifella sp. IM 167
Afifella
Afipia carboxidovorans OM4
Afipia carboxidovorans OM5
Afipia felis ATCC 53690
Afipia felis
Afipia sp. STM 4800
Agaribacterium haliotis
Agaricus bisporus var. bisporus H97
Agaricus bisporus var. burnettii JB137-S8
Agarilytica rhodophyticola
Agarivorans aestuarii
Agarivorans albus MKT 106
Agarivorans albus
Agarivorans gilvus
Agarivorans litoreus
Agarivorans sp. Alg241-V36
Agarivorans sp. B2Z047
Agarivorans sp. TSD2052
Agarivorans sp. Toyoura001
Agarivorans
Agathobacter ruminis
Agathobaculum butyriciproducens
Agathobaculum desmolans
Aggregatilinea lenta
Agitococcus lubricus
Agreia bicolorata
Agreia sp. COWG
Agreia sp. VKM Ac-1783
Agrilactobacillus composti DSM 18527 = JCM 14202
Agrilactobacillus yilanensis
Agrobacterium albertimagni AOL15
Agrobacterium bohemicum
Agrobacterium cavarae
Agrobacterium deltaense
Agrobacterium fabacearum TT111
Agrobacterium fabacearum
Agrobacterium fabrum
Agrobacterium larrymoorei
Agrobacterium radiobacter DSM 30147
Agrobacterium rhizogenes
Agrobacterium rosae
Agrobacterium salinitolerans
Agrobacterium sp. 10MFCol1.1
Agrobacterium sp. 13-2099-1-2
Agrobacterium sp. 1579
Agrobacterium sp. ATCC 31749
Agrobacterium sp. CNPSo 3708
Agrobacterium sp. DSM 25558
Agrobacterium sp. ERR8
Agrobacterium sp. P7-7
Agrobacterium sp. RAC06
Agrobacterium sp. ST15.13.015
Agrobacterium sp. YIC 4121
Agrobacterium sp.
Agrobacterium tumefaciens complex
Agrobacterium tumefaciens
Agrobacterium vaccinii
Agrobacterium vitis
Agrobacterium
Agrococcus jejuensis
Agrococcus sp. ARC_14
Agrococcus sp. SGAir0287
Agromyces allii
Agromyces archimandritae
Agromyces badenianii
Agromyces flavus
Agromyces humatus
Agromyces lapidis
Agromyces seonyuensis
Agromyces sp. C10
Agromyces sp. CF514
Agromyces sp. CFWR-12
Agromyces sp. Leaf222
Agromyces sp. NDB4Y10
Agromyces sp. ORS 1437
Agromyces tardus
Agromyces terreus
Ahrensia sp. R2A130
Albimonas pacifica
Albugo laibachii Nc14
Alcaligenaceae bacterium
Alcaligenes faecalis
Alcanivoracaceae
Alcanivorax hongdengensis
Alcanivorax sp. 24
Alcanivorax sp. 6-D-6
Alcanivorax sp. ALC70
Alcanivorax sp. Alg238-V104
Alcanivorax sp. DG881
Alcanivorax sp. DSM 26293
Alcanivorax sp. DSM 26295
Alcanivorax sp. N3-2A
Alcanivorax sp. NBRC 102024
Alcanivorax sp. VBW004
Alcanivorax sp.
Alcanivorax
Aldersonia kunmingensis
Aldersonia sp. NBC_00410
Algibacter alginicilyticus
Algibacter sp. L1A34
Algicola sagamiensis
Algimonas arctica
Algiphilus aromaticivorans
Algoriphagus hitonicola
Algoriphagus lacus
Aliagarivorans marinus
Aliagarivorans taiwanensis
Aliagarivorans
Aliarcobacter butzleri
Aliarcobacter cibarius
Aliarcobacter cryaerophilus
Aliarcobacter faecis
Aliarcobacter lanthieri
Aliarcobacter skirrowii CCUG 10374
Aliarcobacter skirrowii
Aliarcobacter trophiarum
Alicycliphilus denitrificans BC
Alicycliphilus denitrificans K601
Alicycliphilus denitrificans
Alicycliphilus sp. B1
Alicyclobacillaceae bacterium I2511
Alicyclobacillus acidocaldarius subsp. acidocaldarius DSM 446
Alicyclobacillus acidocaldarius subsp. acidocaldarius Tc-4-1
Alicyclobacillus acidocaldarius
Alicyclobacillus hesperidum URH17-3-68
Alicyclobacillus macrosporangiidus
Alicyclobacillus pomorum
Alicyclobacillus tengchongensis
Alicyclobacillus vulcanalis
Alienimonas californiensis
Alienimonas chondri
Aliicoccus persicus
Aliidiomarina celeris
Aliidiomarina maris
Aliidiomarina taiwanensis
Aliidongia dinghuensis
Aliifodinibius roseus
Aliiglaciecola lipolytica E3
Aliiglaciecola lipolytica
Aliihoeflea sp. 2WW
Aliihoeflea sp. 40Bstr573
Aliikangiella marina
Aliikangiella sp. G2MR2-5
Aliinostoc sp. MN-3B
Aliinostoc sp. MN-6D
Aliinostoc sp. MN-7A
Aliinostoc sp. MN-7B
Aliinostoc sp. MN-7D
Aliinostoc sp. MN-9B
Aliiruegeria sabulilitoris
Aliivibrio salmonicida LFI1238
Aliivibrio
Alishewanella aestuarii B11
Alishewanella agri BL06
Alishewanella agri
Alishewanella jeotgali KCTC 22429
Alishewanella longhuensis
Alishewanella sp. HH-ZS
Alishewanella sp. WH16-1
Alishewanella
Aliterella atlantica
Alkalibacillus haloalkaliphilus
Alkalibacter saccharofermentans DSM 14828
Alkalibacter saccharofermentans
Alkalibaculum sporogenes
Alkalicoccus chagannorensis
Alkalicoccus urumqiensis
Alkaliflexus imshenetskii
Alkalihalobacillus akibai JCM 9157
Alkalihalobacillus akibai
Alkalihalobacillus bogoriensis
Alkalihalobacillus clausii
Alkalihalobacillus hemicellulosilyticus JCM 9152
Alkalihalobacillus hemicellulosilyticus
Alkalihalobacillus macyae
Alkalihalobacillus okhensis
Alkalihalobacillus patagoniensis
Alkalihalobacillus pseudalcaliphilus
Alkalihalobacillus trypoxylicola
Alkalihalobacillus wakoensis JCM 9140
Alkalihalobacillus wakoensis
Alkalihalophilus marmarensis DSM 21297
Alkalihalophilus marmarensis
Alkalihalophilus pseudofirmus OF4
Alkalihalophilus pseudofirmus
Alkalilimnicola ehrlichii MLHE-1
Alkalilimnicola ehrlichii
Alkalilimnicola halodurans
Alkalinema sp. FACHB-956
Alkaliphilus flagellatus
Alkaliphilus metalliredigens QYMF
Alkaliphilus metalliredigens
Alkaliphilus oremlandii OhILAs
Alkaliphilus oremlandii
Alkaliphilus peptidifermentans DSM 18978
Alkaliphilus peptidifermentans
Alkaliphilus pronyensis
Alkaliphilus serpentinus
Alkaliphilus sp.
Alkaliphilus transvaalensis
Alkalispirochaeta alkalica
Alkalispirochaeta americana
Alkalitalea saponilacus
Allisonella histaminiformans
Alloalcanivorax balearicus
Alloalcanivorax dieselolei
Alloalcanivorax gelatiniphagus
Alloalcanivorax marinus
Alloalcanivorax mobilis
Alloalcanivorax venustensis
Alloalcanivorax xenomutans
Allochromatium humboldtianum
Allochromatium minutissimum
Allochromatium palmeri
Allochromatium tepidum
Allochromatium vinosum DSM 180
Allochromatium vinosum
Allochromatium warmingii
Allocoleopsis franciscana PCC 7113
Allocoleopsis franciscana
Allokutzneria albata
Allokutzneria sp. A3M-2-11 16
Allokutzneria sp. NRRL B-24872
Allomeiothermus silvanus
Allonocardiopsis opalescens
Allorhizobium ampelinum S4
Allorhizobium ampelinum
Allorhizobium undicola
Allorhizocola rhizosphaerae
Allosaccharopolyspora coralli
Alphaproteobacteria bacterium BRH_c36
Alphaproteobacteria bacterium CG_4_10_14_0_2_um_filter_63_37
Alphaproteobacteria bacterium HGW-Alphaproteobacteria-12
Alphaproteobacteria bacterium HGW-Alphaproteobacteria-14
Alphaproteobacteria bacterium HGW-Alphaproteobacteria-15
Alphaproteobacteria bacterium HGW-Alphaproteobacteria-18
Alphaproteobacteria bacterium HGW-Alphaproteobacteria-1
Alphaproteobacteria bacterium HGW-Alphaproteobacteria-2
Alphaproteobacteria bacterium HGW-Alphaproteobacteria-4
Alphaproteobacteria bacterium MarineAlpha3_Bin1
Alphaproteobacteria bacterium MarineAlpha3_Bin2
Alphaproteobacteria bacterium MarineAlpha3_Bin6
Alphaproteobacteria bacterium MarineAlpha3_Bin7
Alphaproteobacteria bacterium PA1
Alphaproteobacteria bacterium PA3
Alphaproteobacteria bacterium PA4
Alphaproteobacteria bacterium
Alpinimonas psychrophila
Alteraurantiacibacter aquimixticola
Altererythrobacter epoxidivorans
Altererythrobacter insulae
Altererythrobacter sp. XM-24bin4
Altererythrobacter sp. ZODW24
Alteribacillus persepolensis
Alteribacter aurantiacus
Alteribacter lacisalsi
Alteribacter natronophilus
Altericroceibacterium endophyticum
Altericroceibacterium spongiae
Altericroceibacterium xinjiangense
Alteripontixanthobacter muriae
Alteriqipengyuania abyssalis
Alteriqipengyuania lutimaris
Alteromonadaceae bacterium 2753L.S.0a.02
Alteromonadaceae bacterium Bs31
Alteromonadaceae bacterium TMED101
Alteromonas antoniana
Alteromonas australica
Alteromonas confluentis
Alteromonas facilis
Alteromonas flava
Alteromonas gracilis
Alteromonas halophila
Alteromonas lipolytica
Alteromonas lipotrueae
Alteromonas lipotrueiana
Alteromonas macleodii str. ‘Black Sea 11’
Alteromonas macleodii
Alteromonas mediterranea DE1
Alteromonas mediterranea UM4b
Alteromonas mediterranea UM7
Alteromonas mediterranea
Alteromonas naphthalenivorans
Alteromonas pelagimontana
Alteromonas ponticola
Alteromonas portus
Alteromonas profundi
Alteromonas sediminis
Alteromonas sp. 76-1
Alteromonas sp. ALT199
Alteromonas sp. ASW11-19
Alteromonas sp. B31-7
Alteromonas sp. BL110
Alteromonas sp. K632G
Alteromonas sp. KS69
Alteromonas sp. KUL106
Alteromonas sp. MMG017
Alteromonas sp. Mac1
Alteromonas sp. Mac2
Alteromonas sp. PRIM-21
Alteromonas sp. V450
Alteromonas sp. chi3
Alteromonas stellipolaris LMG 21856
Alteromonas stellipolaris
Alteromonas
Amantichitinum ursilacus
Amaricoccus solimangrovi
Amazonocrinis nigriterrae
Amborella trichopoda
Ameyamaea chiangmaiensis
Aminiphilus circumscriptus
Aminipila butyrica
Aminipila luticellarii
Aminipila terrae
Aminobacter sp. BA135
Aminobacterium colombiense DSM 12261
Aminobacterium colombiense
Aminobacterium mobile
Aminobacterium sp.
Aminomonas paucivorans DSM 12260
Aminomonas paucivorans
Ammonifex degensii KC4
Ammonifex degensii
Ammonifex thiophilus
Amnibacterium flavum
Amnibacterium kyonggiense
Amnibacterium setariae
Amniculibacterium aquaticum
Amniculibacterium sp. G2-70
Amorphomonas oryzae
Amphibacillus cookii
Amphibacillus xylanus NBRC 15112
Amphibacillus xylanus
Amphritea atlantica
Amphritea japonica
Amphritea pacifica
Ampullimonas aquatilis
Amycolatopsis acidicola
Amycolatopsis acididurans
Amycolatopsis acidiphila
Amycolatopsis aidingensis
Amycolatopsis alba
Amycolatopsis albispora
Amycolatopsis arida
Amycolatopsis australiensis
Amycolatopsis azurea
Amycolatopsis benzoatilytica
Amycolatopsis bullii
Amycolatopsis camponoti
Amycolatopsis cihanbeyliensis
Amycolatopsis circi
Amycolatopsis coloradensis
Amycolatopsis dendrobii
Amycolatopsis deserti
Amycolatopsis eburnea
Amycolatopsis echigonensis
Amycolatopsis endophytica
Amycolatopsis granulosa
Amycolatopsis jejuensis
Amycolatopsis jiangsuensis
Amycolatopsis kentuckyensis
Amycolatopsis keratiniphila
Amycolatopsis lexingtonensis
Amycolatopsis lurida
Amycolatopsis marina
Amycolatopsis mediterranei
Amycolatopsis methanolica
Amycolatopsis niigatensis
Amycolatopsis nivea
Amycolatopsis oliviviridis
Amycolatopsis orientalis
Amycolatopsis palatopharyngis
Amycolatopsis pittospori
Amycolatopsis pretoriensis
Amycolatopsis regifaucium
Amycolatopsis rhizosphaerae
Amycolatopsis roodepoortensis
Amycolatopsis rubida
Amycolatopsis saalfeldensis
Amycolatopsis sacchari
Amycolatopsis sp. 195334CR
Amycolatopsis sp. AA4
Amycolatopsis sp. ATCC 39116
Amycolatopsis sp. BJA-103
Amycolatopsis sp. CA-126428
Amycolatopsis sp. CA-128772
Amycolatopsis sp. EV170708-02-1
Amycolatopsis sp. FDAARGOS 1241
Amycolatopsis sp. FU40
Amycolatopsis sp. HUAS 11-8
Amycolatopsis sp. Hca4
Amycolatopsis sp. M39
Amycolatopsis sp. MJM2582
Amycolatopsis sp. MtRt-6
Amycolatopsis sp. OK19-0408
Amycolatopsis sp. Poz14
Amycolatopsis sp. SID8362
Amycolatopsis sp. TNS106
Amycolatopsis sp. WAC 01376
Amycolatopsis sp. WAC 04169
Amycolatopsis sp. WAC 04182
Amycolatopsis sp. WAC 04197
Amycolatopsis sp. WQ 127309
Amycolatopsis sp. YIM 10
Amycolatopsis suaedae
Amycolatopsis sulphurea
Amycolatopsis thailandensis
Amycolatopsis thermalba
Amycolatopsis thermoflava
Amycolatopsis tolypomycina
Amycolatopsis tucumanensis
Amycolatopsis umgeniensis
Amycolatopsis vancoresmycina
Amycolatopsis vastitatis
Amycolatopsis viridis
Amycolatopsis xylanica
Amycolatopsis
Amylibacter sp. SFDW26
Anabaena azotica FACHB-118
Anabaena azotica
Anabaena catenula
Anabaena cf. pirinica ACSSI 153
Anabaena cylindrica PCC 7122
Anabaena cylindrica UTAD_A212
Anabaena cylindrica
Anabaena lutea
Anabaena minutissima
Anabaena oryzae CG14
Anabaena oryzae Ind3
Anabaena oryzae Ind4
Anabaena oscillarioides
Anabaena sp. 4-3
Anabaena sp. 90
Anabaena sp. A2
Anabaena sp. AL93
Anabaena sp. BC1
Anabaena sp. CA = ATCC 33047
Anabaena sp. CCAP 1446/1C
Anabaena sp. CH1
Anabaena sp. CRKS33
Anabaena sp. CS-542/02
Anabaena sp. FACHB-1250
Anabaena sp. FACHB-709
Anabaena sp. FACHB-83
Anabaena sp. I1
Anabaena sp. Ind5
Anabaena sp. Ind6
Anabaena sp. L-31
Anabaena sp. LCRNK_18
Anabaena sp. LCRNK_23
Anabaena sp. LCRNK_27
Anabaena sp. LCRNK_28
Anabaena sp. LCRNK_4
Anabaena sp. LCRNK_6
Anabaena sp. LCRNK_7
Anabaena sp. LCRNK_9
Anabaena sp. LG1
Anabaena sp. LG2
Anabaena sp. MDT14b
Anabaena sp. MN-12C
Anabaena sp. NQAIF321
Anabaena sp. P4-9
Anabaena sp. PA1
Anabaena sp. PCC 7108
Anabaena sp. PCC 9109
Anabaena sp. PO1
Anabaena sp. UHCC 0187
Anabaena sp. UHCC 0204
Anabaena sp. UHCC 0253
Anabaena sp. WA102
Anabaena sp. YBS01
Anabaena sphaerica CG13
Anabaena sphaerica var. tenuis PMC188.03
Anabaena sphaerica var. tenuis PMC229.04
Anabaena sphaerica var. tenuis PMC246.05
Anabaena sphaerica var. tenuis PMC266.06
Anabaena sphaerica
Anabaena subtropica
Anabaena
Anabaenopsis circularis
Anabaenopsis elenkinii CCIBt3563
Anabaenopsis elenkinii
Anabaenopsis sp. Ind8
Anabaenopsis sp. NRE1
Anabarilius grahami
Anaeroarcus burkinensis
Anaerobacillus alkalidiazotrophicus
Anaerobacillus alkalilacustris
Anaerobacillus arseniciselenatis
Anaerobacterium chartisolvens
Anaerobutyricum hallii DSM 3353
Anaerobutyricum hallii
Anaerobutyricum soehngenii
Anaerobutyricum
Anaerococcus prevotii
Anaerococcus tetradius
Anaerocolumna aminovalerica
Anaerocolumna cellulosilytica
Anaerocolumna chitinilytica
Anaerocolumna jejuensis DSM 15929
Anaerocolumna jejuensis
Anaerocolumna sedimenticola
Anaerocolumna xylanovorans DSM 12503
Anaerocolumna xylanovorans
Anaeroglobus geminatus
Anaeroglobus sp. AF13-6AC
Anaerolactibacter massiliensis
Anaerolineaceae bacterium 4572_5.2
Anaerolineaceae bacterium oral taxon 439
Anaerolineaceae bacterium
Anaerolineae bacterium CG03_land_8_20_14_0_80_58_20
Anaerolineae bacterium CG_4_9_14_0_8_um_filter_58_9
Anaerolineae bacterium SG8_19
Anaerolineae bacterium SM23_63
Anaerolineae bacterium SM23_84
Anaerolineae bacterium
Anaerolineales bacterium
Anaeromicrobium sediminis
Anaeromicropila herbilytica
Anaeromicropila populeti
Anaeromusa acidaminophila
Anaeromyces robustus
Anaeromyxobacter dehalogenans 2CP-1
Anaeromyxobacter dehalogenans 2CP-C
Anaeromyxobacter dehalogenans
Anaeromyxobacter diazotrophicus
Anaeromyxobacter oryzae
Anaeromyxobacter paludicola
Anaeromyxobacter sp. Fw109-5
Anaeromyxobacter sp. K
Anaeromyxobacter sp. PSR-1
Anaeromyxobacter sp. SG22
Anaeromyxobacter sp. SG63
Anaeropeptidivorans aminofermentans
Anaerophaga thermohalophila
Anaerosacchariphilus polymeriproducens
Anaerosalibacter massiliensis
Anaerosalibacter sp. Marseille-P3206
Anaerospora hongkongensis
Anaerosporobacter mobilis DSM 15930
Anaerosporobacter mobilis
Anaerosporomusa subterranea
Anaerostipes hadrus
Anaerostipes sp. MSJ-23
Anaerostipes sp. Marseille-Q3525
Anaerostipes
Anaerotalea alkaliphila
Anaerotignum lactatifermentans
Anaerotignum neopropionicum
Anaerotignum propionicum DSM 1682
Anaerotignum propionicum
Anaerotruncus colihominis DSM 17241
Anaerotruncus colihominis
Anaerotruncus sp. AF02-27
Anaerotruncus sp. G3(2012)
Anaerovibrio lipolyticus
Anaerovibrio sp. RM50
Anaerovirgula multivorans
Ananas comosus
Anaplasma marginale
Ancylobacter dichloromethanicus
Ancylobacter gelatini
Ancylomarina subtilis
Ancylothrix sp. D3o
Anditalea andensis
Andreprevotia chitinilytica
Andreprevotia sp. IGB-42
Aneurinibacillus aneurinilyticus ATCC 12856
Aneurinibacillus aneurinilyticus
Aneurinibacillus migulanus
Aneurinibacillus terranovensis
Aneurinibacillus tyrosinisolvens
Anoxybacillus ayderensis
Anoxybacillus flavithermus AK1
Anoxybacillus flavithermus NBRC 109594
Anoxybacillus flavithermus TNO-09.006
Anoxybacillus flavithermus WK1
Anoxybacillus flavithermus
Anoxybacillus gonensis
Anoxybacillus sp. KU2-6(11)
Anoxybacillus suryakundensis
Anoxybacillus tepidamans
Anoxybacillus thermarum
Anoxybacillus
Antarcticibacterium sp. 1MA-6-2
Antarcticibacterium
Antarctobacter heliothermus
Anthocerotibacter panamensis
Anthropogastromicrobium aceti
Antribacter gilvus
Antribacter sp. KLBMP9083
Aphanizomenon flos-aquae 2012/KM1/D3
Aphanizomenon flos-aquae KM1D3_PB
Aphanizomenon flos-aquae LD13
Aphanizomenon flos-aquae MDT14a
Aphanizomenon flos-aquae
Aphanizomenon gracile UADFA10
Aphanizomenon gracile UADFA11
Aphanizomenon gracile UADFA12
Aphanizomenon gracile UADFA16
Aphanizomenon gracile UADFA2
Aphanizomenon sp. CS-733/32
Aphanizomenon sp. KAC15
Aphanizomenon sp. UHCC 0183
Aphanomyces astaci
Aphanomyces invadans
Aphanothece cf. salina LEGE 06149
Aphanothece hegewaldii
Aphanothece sacrum
Aquabacter cavernae
Aquabacter sp. L1I39
Aquabacter spiritensis
Aquabacterium parvum
Aquabacterium pictum
Aquabacterium soli
Aquabacterium sp. A08
Aquabacterium sp. CECT 9606
Aquabacterium sp. J223
Aquabacterium sp. NJ1
Aquabacterium sp. OR-4
Aquabacterium sp.
Aquaspirillum sp. LM1
Aquidulcibacter paucihalophilus
Aquifex aeolicus VF5
Aquificae bacterium
Aquimarina agarivorans
Aquimarina amphilecti
Aquimarina brevivitae
Aquimarina intermedia
Aquimarina litoralis
Aquimarina mytili
Aquimarina sp. AD1
Aquimarina sp. AU474
Aquimarina sp. Aq107
Aquimarina sp. Aq135
Aquimarina sp. BL5
Aquimarina sp. ERC-38
Aquimarina sp. I32.4
Aquimarina sp. U1-2
Aquimarina spongiae
Aquimarina
Aquincola rivuli
Aquincola sp. J276
Aquincola tertiaricarbonis
Aquincola
Aquisphaera giovannonii
Aquisphaera sp. JC669
Aquitalea magnusonii
Aquitalea sp. ASV1
Aquitalea sp. MWU14-2217
Arabidopsis lyrata subsp. lyrata
Arabidopsis lyrata
Arabidopsis thaliana
Arabis alpina
Arachnia propionica F0230a
Arachnia propionica
Arboricoccus pini
Archaeoglobales archaeon
Archaeoglobus fulgidus DSM 4304
Archaeoglobus fulgidus
Archaeoglobus profundus DSM 5631
Archaeoglobus profundus
Archaeoglobus sp.
Archaeoglobus sulfaticallidus PM70-1
Archaeoglobus sulfaticallidus
Archaeoglobus veneficus SNP6
Archaeoglobus veneficus
Archangium gephyra
Archangium sp. CY-1
Archangium sp. Cb G35
Archangium sp. miwbw1
Archangium violaceum
Arcobacter acticola
Arcobacter caeni
Arcobacter cryaerophilus gv. crypticus
Arcobacter defluvii
Arcobacter ellisii
Arcobacter nitrofigilis DSM 7299
Arcobacter nitrofigilis
Arcobacter sp. ARW1-2F2
Arcobacter sp. CECT 8985
Arcobacter sp. CECT 8986
Arcobacter sp. F2176
Arcobacter sp. FWKO B
Arcobacter sp. LA11
Arcobacter sp.
Arcobacter suis CECT 7833
Arcobacter suis
Arcobacter venerupis
Arcobacter
Arcobacteraceae
Arcticibacter svalbardensis MN12-7
Arcticibacter svalbardensis
Arcticibacter tournemirensis
Arenibacter aquaticus
Arenibaculum pallidiluteum
Arenimonas composti
Argonema antarcticum
Argonema galeatum
Armatimonadetes bacterium OLB18
Aromatoleum diolicum
Aromatoleum evansii
Aromatoleum toluclasticum
Aromatoleum tolulyticum
Aromatoleum toluolicum
Aromatoleum toluvorans
Arsenicitalea aurantiaca
Arsukibacterium ikkense
Arsukibacterium sp. MJ3
Artemisia annua
Arthrobacter agilis
Arthrobacter alpinus
Arthrobacter antioxidans
Arthrobacter cavernae
Arthrobacter celericrescens
Arthrobacter cheniae
Arthrobacter citreus
Arthrobacter crusticola
Arthrobacter crystallopoietes
Arthrobacter gallicola
Arthrobacter gandavensis
Arthrobacter glacialis
Arthrobacter koreensis
Arthrobacter livingstonensis
Arthrobacter luteolus
Arthrobacter mobilis
Arthrobacter oryzae
Arthrobacter pigmenti
Arthrobacter pityocampae
Arthrobacter psychrochitiniphilus
Arthrobacter pullicola
Arthrobacter ramosus
Arthrobacter rhombi
Arthrobacter ruber
Arthrobacter silviterrae
Arthrobacter sp. 131MFCol6.1
Arthrobacter sp. 135MFCol5.1
Arthrobacter sp. 18/1
Arthrobacter sp. 18/4
Arthrobacter sp. 24S4-2
Arthrobacter sp. 4R501
Arthrobacter sp. 8AJ
Arthrobacter sp. AFG7.2
Arthrobacter sp. AG258
Arthrobacter sp. AK01
Arthrobacter sp. B0490
Arthrobacter sp. B1805
Arthrobacter sp. BF1
Arthrobacter sp. BL-252-APC-1A
Arthrobacter sp. Bi26
Arthrobacter sp. Br18
Arthrobacter sp. Bz4
Arthrobacter sp. CAL618
Arthrobacter sp. CAN_C5
Arthrobacter sp. CAU 1506
Arthrobacter sp. CCBAU 10866
Arthrobacter sp. CCBAU 10897
Arthrobacter sp. CCBAU 10948
Arthrobacter sp. CJ23
Arthrobacter sp. DWC3
Arthrobacter sp. E3
Arthrobacter sp. ERGS1: 01
Arthrobacter sp. Edens01
Arthrobacter sp. FW306-05-C
Arthrobacter sp. FW306-06-A
Arthrobacter sp. FW306-07-I
Arthrobacter sp. H14-L1
Arthrobacter sp. H20
Arthrobacter sp. H41
Arthrobacter sp. HMWF013
Arthrobacter sp. Helios
Arthrobacter sp. I2-34
Arthrobacter sp. ISL-5
Arthrobacter sp. ISL-69
Arthrobacter sp. KBS0703
Arthrobacter sp. L77
Arthrobacter sp. Leaf141
Arthrobacter sp. Leaf69
Arthrobacter sp. MA-N2
Arthrobacter sp. MSA 4-2
Arthrobacter sp. MYb214
Arthrobacter sp. MYb222
Arthrobacter sp. Marseille-P9274
Arthrobacter sp. N199823
Arthrobacter sp. NBRC 112813
Arthrobacter sp. NamB2
Arthrobacter sp. NtRootA1
Arthrobacter sp. P2b
Arthrobacter sp. PAMC25564
Arthrobacter sp. RIT-PI-e
Arthrobacter sp. RT-1
Arthrobacter sp. SF27
Arthrobacter sp. SLBN-100
Arthrobacter sp. SO3
Arthrobacter sp. SO5
Arthrobacter sp. SX1312
Arthrobacter sp. Soil762
Arthrobacter sp. StoSoilA2
Arthrobacter sp. StoSoilB13
Arthrobacter sp. StoSoilB5
Arthrobacter sp. TB 23
Arthrobacter sp. VKM Ac-2550
Arthrobacter sp. Y81
Arthrobacter sp. ok909
Arthrobacter sp. ov118
Arthrobacter sp. zg-Y324
Arthrobacter sp. zg-Y453
Arthrobacter sp. zg-Y462
Arthrobacter sp. zg-Y916
Arthrobacter terrae
Arthrobacter ulcerisalmonis
Arthrobacter wenxiniae
Arthrobacter zhaoguopingii
Arthrobacter
Asaia bogorensis NBRC 16594
Asaia bogorensis
Asaia platycodi
Asaia siamensis
Asaia sp. W19
Asanoa hainanensis
Asanoa iriomotensis
Ascidiaceihabitans donghaensis
Aspergillus arachidicola
Aspergillus niger
Aspergillus nomiae NRRL 13137
Aspergillus sclerotialis
Aspergillus sp. HF37
Aspergillus steynii IBT 23096
Asticcacaulis solisilvae
Athalassotoga saccharophila
Atlanticothrix silvestris CENA357
Atlanticothrix silvestris CENA368
Atlanticothrix silvestris
Atopomonas hussainii
Aulosira laxa NIES-50
Aulosira sp. FACHB-615
Aurantiacibacter atlanticus
Aurantiacibacter marinus
Aurantiacibacter odishensis
Aurantiacibacter spongiae
Aurantiacibacter suaedae
Aurantiacibacter xanthus
Aurantimicrobium minutum
Aurantimonas marina
Aurantimonas sp. 22II-16-19i
Aurantimonas sp. CCNWGS0021-2
Aureicoccus marinus
Aureimonas altamirensis
Aureimonas endophytica
Aureimonas flava
Aureimonas frigidaquae
Aureimonas leprariae
Aureimonas mangrovi
Aureimonas sp. AU12
Aureimonas sp. AU20
Aureimonas sp. AU40
Aureimonas sp. AU4
Aureimonas sp. D3
Aureimonas sp. Leaf454
Aureimonas sp. N4
Aureimonas sp. SA4125
Aureimonas ureilytica
Austwickia chelonae
Azoarcus communis
Azoarcus indigens
Azoarcus olearius
Azoarcus sp. Aa7
Azoarcus sp. CIB
Azoarcus sp. DD4
Azoarcus sp. DN11
Azoarcus sp. KH32C
Azoarcus sp. L1K30
Azoarcus sp. P3-4
Azoarcus sp. P3-6
Azoarcus sp. PHD
Azoarcus sp. TTM-91
Azoarcus sp.
Azohydromonas aeria
Azohydromonas australica
Azohydromonas caseinilytica
Azohydromonas lata
Azohydromonas sediminis
Azohydromonas sp. P4-12
Azomonas agilis
Azomonas macrocytogenes
Azonexus caeni
Azonexus fungiphilus
Azonexus hydrophilus DSM 23864
Azonexus hydrophilus
Azonexus sp. R2A-61
Azorhizobium caulinodans ORS 571
Azorhizobium caulinodans
Azorhizobium doebereinerae
Azorhizobium oxalatiphilum
Azorhizobium sp. 32-67-21
Azorhizobium sp. 35-67-15
Azorhizobium sp. AG788
Azorhizobium sp. KNUC169
Azorhizobium sp. MM5717
Azorhizobium sp. SA12
Azorhizobium
Azospira inquinata
Azospira oryzae PS
Azospira oryzae
Azospira restricta
Azospira sp. I09
Azospira sp. I13
Azospira
Azospirillum agricola
Azospirillum argentinense
Azospirillum baldaniorum
Azospirillum brasilense
Azospirillum canadense
Azospirillum cavernae
Azospirillum doebereinerae
Azospirillum fermentarium
Azospirillum formosense
Azospirillum halopraeferens
Azospirillum himalayense
Azospirillum humicireducens
Azospirillum lipoferum 4B
Azospirillum lipoferum
Azospirillum melinis
Azospirillum oleiclasticum
Azospirillum oryzae
Azospirillum palustre
Azospirillum picis
Azospirillum ramasamyi
Azospirillum rugosum
Azospirillum soli
Azospirillum sp. 412522
Azospirillum sp. 47_25
Azospirillum sp. A1-3
Azospirillum sp. B21
Azospirillum sp. B4
Azospirillum sp. B506
Azospirillum sp. B510
Azospirillum sp. CAG: 239
Azospirillum sp. CAG: 260
Azospirillum sp. CC-RW20-51
Azospirillum sp. P3-30
Azospirillum sp. P5-22
Azospirillum sp. P6-20
Azospirillum sp. RU37A
Azospirillum sp. RU38E
Azospirillum sp. SYSU D00513
Azospirillum sp. Sh1
Azospirillum sp. Sp 7
Azospirillum sp. TN01
Azospirillum sp. TN02
Azospirillum sp. TN03
Azospirillum sp. TN09
Azospirillum sp. TN12
Azospirillum sp. TN13
Azospirillum sp. TN15
Azospirillum sp. TN22
Azospirillum sp. TN30
Azospirillum sp. TN31
Azospirillum sp. TSA10
Azospirillum sp. TSA11
Azospirillum sp. TSA12
Azospirillum sp. TSA13
Azospirillum sp. TSA14w
Azospirillum sp. TSA15c
Azospirillum sp. TSA16
Azospirillum sp. TSA18
Azospirillum sp. TSA19
Azospirillum sp. TSA20c
Azospirillum sp. TSA2s
Azospirillum sp. TSA36t
Azospirillum sp. TSA6c
Azospirillum sp. TSH100
Azospirillum sp. TSH10
Azospirillum sp. TSH19
Azospirillum sp. TSH20
Azospirillum sp. TSH21
Azospirillum sp. TSH2
Azospirillum sp. TSH58
Azospirillum sp. TSH5
Azospirillum sp. TSH64
Azospirillum sp. TSH6
Azospirillum sp. TSH78
Azospirillum sp. TSH7
Azospirillum sp. TSH81w
Azospirillum sp. TSH96
Azospirillum sp. TSO22-1
Azospirillum sp. TSO35-2
Azospirillum sp. TSO5
Azospirillum sp.
Azospirillum thermophilum
Azospirillum thiophilum
Azospirillum zeae
Azospirillum
Azotobacter armeniacus
Azotobacter beijerinckii
Azotobacter bryophylli
Azotobacter chroococcum NCIMB 8003
Azotobacter chroococcum subsp. isscasi
Azotobacter chroococcum
Azotobacter nigricans subsp. nigricans
Azotobacter salinestris
Azotobacter sp. DVD-7
Azotobacter sp. DVD-8
Azotobacter sp. GVT-10
Azotobacter sp. GVT-6
Azotobacter sp. P6-14
Azotobacter sp. P7-20
Azotobacter sp. P8-14
Azotobacter sp. P8-26
Azotobacter sp. RCR-11
Azotobacter tropicalis
Azotobacter vinelandii CA6
Azotobacter vinelandii CA
Azotobacter vinelandii DJ
Azotobacter vinelandii
Azotobacter
Azovibrio restrictus
Baaleninema simplex
Bacillaceae bacterium SAS-127
Bacillaceae
Bacillales
Bacilli bacterium
Bacilliculturomica massiliensis
Bacillus aerophilus
Bacillus altitudinis
Bacillus alveayuensis
Bacillus amyloliquefaciens
Bacillus andreraoultii
Bacillus anthracis
Bacillus atrophaeus C89
Bacillus atrophaeus
Bacillus australimaris
Bacillus badius
Bacillus cereus ATCC 4342
Bacillus cereus VD118
Bacillus cereus VDM053
Bacillus cereus
Bacillus cihuensis
Bacillus coahuilensis m2-6
Bacillus coahuilensis p1.1.43
Bacillus coahuilensis
Bacillus dakarensis
Bacillus gobiensis
Bacillus halotolerans
Bacillus haynesii
Bacillus inaquosorum KCTC 13429
Bacillus infantis NRRL B-14911
Bacillus intestinalis
Bacillus licheniformis LMG 17339
Bacillus licheniformis LMG 6934
Bacillus licheniformis LMG 7559
Bacillus licheniformis WX-02
Bacillus licheniformis
Bacillus massiliigorillae
Bacillus massilionigeriensis
Bacillus methanolicus MGA3
Bacillus methanolicus PB1
Bacillus methanolicus
Bacillus mojavensis
Bacillus nakamurai
Bacillus ndiopicus
Bacillus niameyensis
Bacillus paralicheniformis
Bacillus phage Spock
Bacillus pseudomycoides
Bacillus pumilus ATCC 7061
Bacillus pumilus
Bacillus safensis FO-36b
Bacillus safensis
Bacillus siamensis
Bacillus smithii 7_3_47FAA
Bacillus smithii
Bacillus sp. (in: firmicutes)
Bacillus sp. 03113
Bacillus sp. 17376
Bacillus sp. 1NLA3E
Bacillus sp. A053
Bacillus sp. AFS006103
Bacillus sp. AFS037270
Bacillus sp. AFS076308
Bacillus sp. AM 13(2015)
Bacillus sp. BN4
Bacillus sp. BT97
Bacillus sp. CCBAU 15518
Bacillus sp. CCBAU 15524
Bacillus sp. CHD6a
Bacillus sp. CPSM8
Bacillus sp. EB600
Bacillus sp. FJAT-18019
Bacillus sp. FJAT-21945
Bacillus sp. FJAT-22090
Bacillus sp. FJAT-26652
Bacillus sp. FJAT-27231
Bacillus sp. FJAT-27245
Bacillus sp. FJAT-27445
Bacillus sp. FJAT-28004
Bacillus sp. G1(2015b)
Bacillus sp. GBSW2
Bacillus sp. I-2
Bacillus sp. ISA1808
Bacillus sp. J33
Bacillus sp. JCM 19034
Bacillus sp. JFL15
Bacillus sp. LK10
Bacillus sp. LL01
Bacillus sp. LLTC93
Bacillus sp. Leaf406
Bacillus sp. Leaf75
Bacillus sp. M 2-6
Bacillus sp. M4
Bacillus sp. M6-12
Bacillus sp. NN21
Bacillus sp. NN31(2014)
Bacillus sp. NRRL B-14911
Bacillus sp. Nf3
Bacillus sp. OV194
Bacillus sp. OxB-1
Bacillus sp. R2(2015)
Bacillus sp. Root147
Bacillus sp. Root239
Bacillus sp. SDLI1
Bacillus sp. SG-1
Bacillus sp. SJS
Bacillus sp. TH008
Bacillus sp. TS-2
Bacillus sp. UNC69MF
Bacillus sp. V3-13
Bacillus sp. VT-16-64
Bacillus sp. WP8
Bacillus sp. X1(2014)
Bacillus sp. Y1
Bacillus sp. YN15
Bacillus sp. ZN14
Bacillus sp. ZN17
Bacillus sp. ZN1
Bacillus sp. ZN20
Bacillus sp. ZN3
Bacillus sp. ZN5
Bacillus sp. ZN7
Bacillus sp. ZN8
Bacillus sp. ZN9
Bacillus sp. c6
Bacillus sp. w5
Bacillus stratosphericus LAMA 585
Bacillus stratosphericus
Bacillus subtilis XF-1
Bacillus subtilis subsp. subtilis str. RO-NN-1
Bacillus subtilis subsp. subtilis str. SC-8
Bacillus subtilis
Bacillus testis
Bacillus thermotolerans
Bacillus thuringiensis MC28
Bacillus thuringiensis
Bacillus timonensis
Bacillus toyonensis
Bacillus velezensis
Bacillus wiedmannii
Bacillus wudalianchiensis
Bacillus xiamenensis
Bacillus xiapuensis
Bacillus zhangzhouensis
Bacillus
Bacteriophage sp.
Bacteroidales bacterium KA00344
Bacteroidales bacterium WCE2008
Bacteroidales bacterium
Bacteroides faecis
Bacteroides fragilis
Bacteroides graminisolvens DSM 19988 = JCM 15093
Bacteroides graminisolvens
Bacteroides ihuae
Bacteroides intestinalis
Bacteroides luti
Bacteroides ovatus
Bacteroides pectinophilus CAG: 437
Bacteroides pyogenes F0041
Bacteroides reticulotermitis JCM 10512
Bacteroides reticulotermitis
Bacteroides sp. SM23_62
Bacteroides sp. SM23_62_1
Bacteroides thetaiotaomicron
Bacteroides uniformis
Bacteroides xylanisolvens
Bacteroidetes bacterium 4484_276
Bacteroidetes bacterium 4572_77
Bacteroidetes bacterium ADurb.Bin217
Bacteroidetes bacterium B1(2017)
Bacteroidetes bacterium CG18_big_fil_WC_8_21_14_2_50_41_14
Bacteroidetes bacterium CG_4_10_14_3_um_filter_42_6
Bacteroidetes bacterium CG_4_9_14_3_um_filter_41_19
Bacteroidetes bacterium GWE2_42_24
Bacteroidetes bacterium GWF2_43_11
Bacteroidetes bacterium HGW-Bacteroidetes-11
Bacteroidetes bacterium HGW-Bacteroidetes-16
Bacteroidetes bacterium HGW-Bacteroidetes-21
Bacteroidetes bacterium HGW-Bacteroidetes-22
Bacteroidetes bacterium HGW-Bacteroidetes-9
Bacteroidetes bacterium QH_7_62_13
Bacteroidetes bacterium QH_8_67_23
Bacteroidetes bacterium QH_9_67_14
Bacteroidetes bacterium QS_3_64_15
Bacteroidetes bacterium QS_7_67_15
Bacteroidetes bacterium QS_8_68_15
Bacteroidetes bacterium SW_4_67_19
Bacteroidia bacterium
Bacteroidota bacterium
Baekduia soli
Baekduia sp. 0141 2
Bahamian heterocystous cyanobacterium CIC5
Baileyella intestinalis
Balneatrix alpica
Balneola vulgaris
Barnesiella propionica
Barnesiella sp. GGCC_0306
Barrientosiimonas humi
Bartonella sp. 114
Bartonella sp. 11B
Bartonella sp. Coyote 22sub2
Batillaria attramentaria
Bdellovibrio bacteriovorus HD100
Bdellovibrio bacteriovorus W
Bdellovibrio bacteriovorus str. Tiberius
Bdellovibrio bacteriovorus
Bdellovibrio sp. ArHS
Bdellovibrio sp. CG10_big_fil_rev_8_21_14_0_10_47_8
Bdellovibrio sp. CG11_big_fil_rev_8_21_14_0_20_39_38
Bdellovibrio sp. CG12_big_fil_rev_8_21_14_0_65_39_13
Bdellovibrio sp. CG22_combo_CG10-13_8_21_14_all_39_27
Bdellovibrio sp. CG_4_9_14_3_um_filter_39_7
Bdellovibrio sp. SKB1291214
Bdellovibrio sp.
Bdellovibrionales bacterium CG11_big_fil_rev_8_21_14_0_20_38_13
Bdellovibrionales bacterium CG12_big_fil_rev_8_21_14_0_65_38_15
Bdellovibrionales bacterium CG22_combo_CG10-13_8_21_14_all_38_13
Beduini massiliensis
Beggiatoa alba B18LD
Beggiatoa alba
Beggiatoa leptomitoformis
Beggiatoa sp. 4572_84
Beijerinckia derxii subsp. derxii
Beijerinckia derxii subsp.
Beijerinckia indica subsp. indica ATCC 9039
Beijerinckia indica subsp. indica
Beijerinckia indica subsp. lacticogenes
Beijerinckia indica
Beijerinckia mobilis
Beijerinckia sp. L45
Beijerinckiaceae bacterium
Belnapia arida
Belnapia moabensis
Belnapia mucosa
Belnapia rosea
Bergeyella zoohelcum
Bermanella marisrubri
Berryella wangjianweii
Betaproteobacteria bacterium CG2_30_59_46
Betaproteobacteria bacterium HGW-Betaproteobacteria-11
Betaproteobacteria bacterium HGW-Betaproteobacteria-12
Betaproteobacteria bacterium HGW-Betaproteobacteria-13
Betaproteobacteria bacterium HGW-Betaproteobacteria-14
Betaproteobacteria bacterium HGW-Betaproteobacteria-15
Betaproteobacteria bacterium HGW-Betaproteobacteria-17
Betaproteobacteria bacterium HGW-Betaproteobacteria-1
Betaproteobacteria bacterium HGW-Betaproteobacteria-22
Betaproteobacteria bacterium HGW-Betaproteobacteria-3
Betaproteobacteria bacterium HGW-Betaproteobacteria-5
Betaproteobacteria bacterium HGW-Betaproteobacteria-7
Betaproteobacteria bacterium HGW-Betaproteobacteria-8
Betaproteobacteria bacterium HGW-Betaproteobacteria-9
Betaproteobacteria bacterium TMED100
Betaproteobacteria bacterium TMED156
Betaproteobacteria bacterium TMED41
Betaproteobacteria bacterium TMED82
Betaproteobacteria bacterium
Bhargavaea ginsengi
Bianquea renquensis
Bifidobacteriaceae bacterium NR015
Bifidobacterium adolescentis ATCC 15703
Bifidobacterium adolescentis
Bifidobacterium aemilianum
Bifidobacterium animalis subsp. animalis ATCC 25527
Bifidobacterium animalis subsp. animalis IM386
Bifidobacterium animalis subsp. animalis MCC 1489
Bifidobacterium animalis subsp. animalis
Bifidobacterium animalis
Bifidobacterium anseris
Bifidobacterium asteroides DSM 20089
Bifidobacterium asteroides PRL2011
Bifidobacterium asteroides
Bifidobacterium boum
Bifidobacterium breve
Bifidobacterium callitrichos
Bifidobacterium catenulatum
Bifidobacterium choerinum
Bifidobacterium commune
Bifidobacterium criceti
Bifidobacterium italicum
Bifidobacterium longum NCC2705
Bifidobacterium parmae
Bifidobacterium pseudocatenulatum CAG: 263
Bifidobacterium pseudocatenulatum DSM 20438 = JCM 1200 = LMG 10505
Bifidobacterium pseudolongum subsp. globosum
Bifidobacterium pseudolongum
Bifidobacterium sp. wkB338
Bifidobacterium sp. wkB344
Bifidobacterium thermophilum
Bifidobacterium xylocopae
Bifidobacterium
Bilifractor porci
Bilophila sp. 4_1_30
Bilophila wadsworthia 3_1_6
Bilophila wadsworthia
Bison bison bison
Bizionia
Blastochloris sulfoviridis
Blastochloris tepida
Blastochloris viridis
Blastococcus endophyticus
Blastococcus saxobsidens
Blastococcus sp. CPCC 205250
Blastococcus sp. CT_GayMR19
Blastococcus sp. CT_GayMR20
Blastococcus sp. DSM 46786
Blastococcus sp. DSM 46838
Blastococcus sp. KM273128
Blastococcus sp. KM273129
Blastococcus sp. TF02A-26
Blastococcus sp. URHD0036
Blastococcus sp. VKM Ac-2987
Blastococcus xanthinilyticus
Blastomonas fulva
Blastomonas natatoria
Blastomonas sp. AAP25
Blastomonas sp. RAC04
Blastomonas
Blastopirellula marina
Blastopirellula sp. J2-11
Blastopirellula sp.
Blautia faecis
Blautia glucerasea
Blautia hydrogenotrophica CAG: 147
Blautia hydrogenotrophica DSM 10507
Blautia hydrogenotrophica
Blautia intestinalis
Blautia luti
Blautia massiliensis
Blautia obeum A2-162
Blautia obeum ATCC 29174
Blautia obeum
Blautia schinkii
Blautia sp. AF19-10LB
Blautia sp. BCRC 81119
Blautia sp. BIOML-A1
Blautia sp. CAG: 237
Blautia sp. CLA-AA-H217
Blautia sp. CLA-AA-H275
Blautia sp. KLE 1732
Blautia sp. KLE_1732_HM_1032
Blautia sp. MCC270
Blautia sp. MSJ-19
Blautia sp. MSJ-36
Blautia sp. MSJ-9
Blautia sp. MSK.20.85
Blautia sp. MSK.21.1
Blautia sp. MSK20_18
Blautia sp. Marseille-P2398
Blautia sp. Marseille-P3087
Blautia sp. OF03-15BH
Blautia sp. OM07-19
Blautia sp. SC05B48
Blautia sp. SF-50
Blautia sp. aa_0143
Blautia wexlerae
Blautia
Blennothrix majus RU2007
Bodo saltans virus
Bordetella ansorpii
Bordetella bronchialis
Bordetella bronchiseptica
Bordetella flabilis
Bordetella genomosp. 10
Bordetella genomosp. 11
Bordetella genomosp. 13
Bordetella genomosp. 1
Bordetella genomosp. 4
Bordetella genomosp. 5
Bordetella genomosp. 8
Bordetella genomosp. 9
Bordetella hinzii
Bordetella pertussis
Bordetella petrii
Bordetella sp. H567
Bordetella sp. N
Borrelia anserina BA2
Borrelia coriaceae ATCC 43381
Borrelia coriaceae
Borrelia crocidurae DOU
Borrelia crocidurae str. Achema
Borrelia crocidurae
Borrelia duttonii CR2A
Borrelia duttonii Ly
Borrelia duttonii
Borrelia hermsii CC1
Borrelia hermsii DAH
Borrelia hermsii HS1
Borrelia hermsii MTW
Borrelia hermsii YBT
Borrelia hermsii YOR
Borrelia hermsii
Borrelia hispanica
Borrelia lonestari
Borrelia miyamotoi FR64b
Borrelia miyamotoi LB-2001
Borrelia parkeri HR1
Borrelia parkeri SLO
Borrelia parkeri
Borrelia persica
Borrelia recurrentis A1
Borrelia turicatae 91E135
Borrelia turicatae
Borrelia
Borreliella afzelii ACA-1
Borreliella afzelii HLJ01
Borreliella afzelii K78
Borreliella afzelii PKo
Borreliella afzelii Tom3107
Borreliella afzelii
Borreliella bissettiae DN127
Borreliella bissettiae
Borreliella burgdorferi
Borreliella chilensis
Borreliella garinii BgVir
Borreliella garinii Far04
Borreliella garinii NMJW1
Borreliella garinii PBr
Borreliella garinii SZ
Borreliella garinii
Borreliella spielmanii A14S
Borreliella spielmanii
Borreliella valaisiana Tom4006
Borreliella valaisiana VS116
Borreliella valaisiana
Bos indicus
Bosea eneae 34614
Bosea lathyri
Bosea lupini
Bosea massiliensis 63287
Bosea robiniae
Bosea sp. 12-68-7
Bosea sp. AK1
Bosea sp. ANAM02
Bosea sp. ASV33
Bosea sp. BH3
Bosea sp. BK604
Bosea sp. CRIB-10
Bosea sp. Leaf344
Bosea sp. RAC05
Bosea sp. Root670
Bosea sp. TND4EK4
Bosea sp. WAO
Bosea sp.
Bosea thiooxidans
Bosea vaviloviae
Bosea vestrisii 34635
Botrimarina colliarenosi
Botrimarina hoheduenensis
Botryobacter ruber
Botryococcus braunii Showa
Bowmanella dokdonensis
Brachybacterium halotolerans
Brachybacterium massiliense
Brachybacterium muris UCD-AY4
Brachybacterium muris
Brachybacterium phenoliresistens
Brachybacterium saurashtrense
Brachybacterium sp. ACRRE
Brachybacterium sp. P6-10-X1
Brachybacterium squillarum
Brachypodium distachyon
Brachyspira alvinipulli
Brachyspira hampsonii
Brachyspira hyodysenteriae
Brachyspira innocens
Brachyspira intermedia PWS/A
Brachyspira intermedia
Brachyspira pilosicoli 95/1000
Brachyspira pilosicoli B2904
Brachyspira pilosicoli SP16
Brachyspira pilosicoli
Brachyspira suanatina
Bradymonas sediminis
Bradyrhizobiaceae bacterium
Bradyrhizobium acaciae
Bradyrhizobium aeschynomenes
Bradyrhizobium agreste
Bradyrhizobium algeriense
Bradyrhizobium altum
Bradyrhizobium americanum
Bradyrhizobium amphicarpaeae
Bradyrhizobium arachidis
Bradyrhizobium archetypum
Bradyrhizobium australiense
Bradyrhizobium barranii subsp. apii
Bradyrhizobium barranii subsp. barranii
Bradyrhizobium barranii
Bradyrhizobium betae
Bradyrhizobium brasilense
Bradyrhizobium campsiandrae
Bradyrhizobium canariense bv. genistearum
Bradyrhizobium canariense
Bradyrhizobium cenepequi
Bradyrhizobium centrolobii
Bradyrhizobium centrosematis
Bradyrhizobium cosmicum
Bradyrhizobium cytisi
Bradyrhizobium dagingense
Bradyrhizobium denitrificans
Bradyrhizobium diazoefficiens SEMIA 5080
Bradyrhizobium diazoefficiens USDA 110
Bradyrhizobium diazoefficiens
Bradyrhizobium diversitatis
Bradyrhizobium elkanii CCBAU 05737
Bradyrhizobium elkanii USDA 61
Bradyrhizobium elkanii USDA 76
Bradyrhizobium elkanii
Bradyrhizobium embrapense
Bradyrhizobium erythrophlei
Bradyrhizobium ferriligni
Bradyrhizobium forestalis
Bradyrhizobium frederickii
Bradyrhizobium ganzhouense
Bradyrhizobium genomosp. III
Bradyrhizobium genomosp. II
Bradyrhizobium genosp. B
Bradyrhizobium genosp. ETH1
Bradyrhizobium genosp. ETH2
Bradyrhizobium genosp. SA-2
Bradyrhizobium genosp. SA-3
Bradyrhizobium genosp. TUXTLAS-10
Bradyrhizobium genosp. TUXTLAS-14
Bradyrhizobium genosp. TUXTLAS-15
Bradyrhizobium genosp. TUXTLAS-17
Bradyrhizobium genosp. TUXTLAS-18
Bradyrhizobium genosp. TUXTLAS-19
Bradyrhizobium genosp. TUXTLAS-1
Bradyrhizobium genosp. TUXTLAS-20
Bradyrhizobium genosp. TUXTLAS-21
Bradyrhizobium genosp. TUXTLAS-22
Bradyrhizobium genosp. TUXTLAS-23
Bradyrhizobium genosp. TUXTLAS-29
Bradyrhizobium genosp. TUXTLAS-2
Bradyrhizobium genosp. TUXTLAS-31
Bradyrhizobium genosp. TUXTLAS-4
Bradyrhizobium genosp. TUXTLAS-5
Bradyrhizobium genosp. TUXTLAS-6
Bradyrhizobium genosp. TUXTLAS-7
Bradyrhizobium genosp. alpha bv. genistearum
Bradyrhizobium glycinis
Bradyrhizobium guangdongense
Bradyrhizobium guangxiense
Bradyrhizobium guangzhouense
Bradyrhizobium hipponense
Bradyrhizobium huanghuaihaiense
Bradyrhizobium icense
Bradyrhizobium ingae
Bradyrhizobium iriomotense
Bradyrhizobium ivorense
Bradyrhizobium japonicum CCBAU 25435
Bradyrhizobium japonicum CCBAU 83623
Bradyrhizobium japonicum SEMIA 5079
Bradyrhizobium japonicum USDA 6
Bradyrhizobium japonicum bv. genistearum
Bradyrhizobium japonicum bv. glycinearum
Bradyrhizobium japonicum
Bradyrhizobium jicamae
Bradyrhizobium kavangense
Bradyrhizobium lablabi
Bradyrhizobium liaoningense CCBAU 83689
Bradyrhizobium liaoningense bv. glycinearum
Bradyrhizobium liaoningense
Bradyrhizobium lupini
Bradyrhizobium macuxiense
Bradyrhizobium manausense
Bradyrhizobium mercantei
Bradyrhizobium namibiense
Bradyrhizobium nanningense
Bradyrhizobium neotropicale
Bradyrhizobium niftali
Bradyrhizobium nitroreducens
Bradyrhizobium oligotrophicum S58
Bradyrhizobium oligotrophicum
Bradyrhizobium ottawaense
Bradyrhizobium pachyrhizi
Bradyrhizobium paxllaeri
Bradyrhizobium quebecense
Bradyrhizobium retamae
Bradyrhizobium rifense
Bradyrhizobium sacchari
Bradyrhizobium sediminis
Bradyrhizobium septentrionale
Bradyrhizobium shewense
Bradyrhizobium sp. 1-7
Bradyrhizobium sp. 105S4
Bradyrhizobium sp. 106S4
Bradyrhizobium sp. 108a1
Bradyrhizobium sp. 114d
Bradyrhizobium sp. 123b
Bradyrhizobium sp. 125e
Bradyrhizobium sp. 12S1MB
Bradyrhizobium sp. 131
Bradyrhizobium sp. 134S2
Bradyrhizobium sp. 141
Bradyrhizobium sp. 146S4
Bradyrhizobium sp. 148S4
Bradyrhizobium sp. 14AB
Bradyrhizobium sp. 153
Bradyrhizobium sp. 155
Bradyrhizobium sp. 157
Bradyrhizobium sp. 15LBIV
Bradyrhizobium sp. 165S4
Bradyrhizobium sp. 168
Bradyrhizobium sp. 16LBIV
Bradyrhizobium sp. 170
Bradyrhizobium sp. 172S4
Bradyrhizobium sp. 172
Bradyrhizobium sp. 183
Bradyrhizobium sp. 184
Bradyrhizobium sp. 186
Bradyrhizobium sp. 187
Bradyrhizobium sp. 18C-1-1
Bradyrhizobium sp. 18C-2-1
Bradyrhizobium sp. 190
Bradyrhizobium sp. 191
Bradyrhizobium sp. 192
Bradyrhizobium sp. 195
Bradyrhizobium sp. 1B1-1
Bradyrhizobium sp. 1S2
Bradyrhizobium sp. 1S4
Bradyrhizobium sp. 2-13
Bradyrhizobium sp. 200
Bradyrhizobium sp. 21B2-1
Bradyrhizobium sp. 21LBIV
Bradyrhizobium sp. 21
Bradyrhizobium sp. 22LBI
Bradyrhizobium sp. 2309
Bradyrhizobium sp. 24S4
Bradyrhizobium sp. 25S4
Bradyrhizobium sp. 26-1-1
Bradyrhizobium sp. 26-3-1
Bradyrhizobium sp. 27S4
Bradyrhizobium sp. 28S4
Bradyrhizobium sp. 29d
Bradyrhizobium sp. 2S1
Bradyrhizobium sp. 2S2
Bradyrhizobium sp. 2S4
Bradyrhizobium sp. 2
Bradyrhizobium sp. 3-2
Bradyrhizobium sp. 30-2-1
Bradyrhizobium sp. 31-1-1
Bradyrhizobium sp. 31S4
Bradyrhizobium sp. 334S2
Bradyrhizobium sp. 33S3
Bradyrhizobium sp. 342S2
Bradyrhizobium sp. 355S2
Bradyrhizobium sp. 359S2
Bradyrhizobium sp. 35S1
Bradyrhizobium sp. 36-1-1
Bradyrhizobium sp. 363S2
Bradyrhizobium sp. 36S2
Bradyrhizobium sp. 3LBC
Bradyrhizobium sp. 3LBIV
Bradyrhizobium sp. 4-8
Bradyrhizobium sp. 40-2-2
Bradyrhizobium sp. 40-3-1
Bradyrhizobium sp. 41-1-2
Bradyrhizobium sp. 41S5
Bradyrhizobium sp. 42-1-1
Bradyrhizobium sp. 43S1
Bradyrhizobium sp. 44-1-1
Bradyrhizobium sp. 45-1-4
Bradyrhizobium sp. 46-1-1
Bradyrhizobium sp. 47-1-1
Bradyrhizobium sp. 48a
Bradyrhizobium sp. 49-2-1
Bradyrhizobium sp. 49b
Bradyrhizobium sp. 4
Bradyrhizobium sp. 51-1-3
Bradyrhizobium sp. 55a
Bradyrhizobium sp. 57S1
Bradyrhizobium sp. 6(2017)
Bradyrhizobium sp. 6-8
Bradyrhizobium sp. 60S1
Bradyrhizobium sp. 60
Bradyrhizobium sp. 61A124
Bradyrhizobium sp. 61S1
Bradyrhizobium sp. 70b2
Bradyrhizobium sp. 98S1MB
Bradyrhizobium sp. 9LBC
Bradyrhizobium sp. 9S1MB
Bradyrhizobium sp. 953
Bradyrhizobium sp. A19
Bradyrhizobium sp. Al
Bradyrhizobium sp. AC87j1
Bradyrhizobium sp. ADIT2-3-1
Bradyrhizobium sp. AG14
Bradyrhizobium sp. AG48
Bradyrhizobium sp. ARR65
Bradyrhizobium sp. AS23.2
Bradyrhizobium sp. AT1
Bradyrhizobium sp. AUGA SZCCT0283
Bradyrhizobium sp. Ad261
Bradyrhizobium sp. Alb1-B-2
Bradyrhizobium sp. Alb1-C-1
Bradyrhizobium sp. Alb2-B-3
Bradyrhizobium sp. Alb3-2-1
Bradyrhizobium sp. Alb4-1-3
Bradyrhizobium sp. Alb4-1-4
Bradyrhizobium sp. Alb4-1-5
Bradyrhizobium sp. Alb5-2-6
Bradyrhizobium sp. ApB16
Bradyrhizobium sp. ApB8
Bradyrhizobium sp. ApCV2
Bradyrhizobium sp. ApE4.8
Bradyrhizobium sp. ApT12
Bradyrhizobium sp. ApT2
Bradyrhizobium sp. Aust11c
Bradyrhizobium sp. Aust13C
Bradyrhizobium sp. BR 10261
Bradyrhizobium sp. BR 10289
Bradyrhizobium sp. BR 3262
Bradyrhizobium sp. BR 3267
Bradyrhizobium sp. BRP22
Bradyrhizobium sp. BRUESC1014
Bradyrhizobium sp. BRUESC119
Bradyrhizobium sp. BRUESC22
Bradyrhizobium sp. BRUESC955
Bradyrhizobium sp. BRUESC960
Bradyrhizobium sp. BRUESC965
Bradyrhizobium sp. BRUESC967
Bradyrhizobium sp. BRUESC968
Bradyrhizobium sp. BTAi1
Bradyrhizobium sp. BtLT13
Bradyrhizobium sp. BtLT4
Bradyrhizobium sp. BtRI18
Bradyrhizobium sp. BtRI3
Bradyrhizobium sp. BtRI4
Bradyrhizobium sp. C-145
Bradyrhizobium sp. C2
Bradyrhizobium sp. C6
Bradyrhizobium sp. C8(2014)
Bradyrhizobium sp. C9
Bradyrhizobium sp. CB1015
Bradyrhizobium sp. CB1024
Bradyrhizobium sp. CB3035
Bradyrhizobium sp. CCBAU 03335
Bradyrhizobium sp. CCBAU 03416
Bradyrhizobium sp. CCBAU 051011
Bradyrhizobium sp. CCBAU 051143
Bradyrhizobium sp. CCBAU 051144
Bradyrhizobium sp. CCBAU 051155
Bradyrhizobium sp. CCBAU 051156
Bradyrhizobium sp. CCBAU 051157
Bradyrhizobium sp. CCBAU 051161
Bradyrhizobium sp. CCBAU 05449
Bradyrhizobium sp. CCBAU 05525
Bradyrhizobium sp. CCBAU 05585
Bradyrhizobium sp. CCBAU 05588
Bradyrhizobium sp. CCBAU 05623
Bradyrhizobium sp. CCBAU 05656
Bradyrhizobium sp. CCBAU 05716
Bradyrhizobium sp. CCBAU 05727
Bradyrhizobium sp. CCBAU 05737
Bradyrhizobium sp. CCBAU 101057
Bradyrhizobium sp. CCBAU 101058
Bradyrhizobium sp. CCBAU 101059
Bradyrhizobium sp. CCBAU 101060
Bradyrhizobium sp. CCBAU 101061
Bradyrhizobium sp. CCBAU 101062
Bradyrhizobium sp. CCBAU 101063
Bradyrhizobium sp. CCBAU 101064
Bradyrhizobium sp. CCBAU 101065
Bradyrhizobium sp. CCBAU 101066
Bradyrhizobium sp. CCBAU 101067
Bradyrhizobium sp. CCBAU 101068
Bradyrhizobium sp. CCBAU 101069
Bradyrhizobium sp. CCBAU 101070
Bradyrhizobium sp. CCBAU 101071
Bradyrhizobium sp. CCBAU 101072
Bradyrhizobium sp. CCBAU 101073
Bradyrhizobium sp. CCBAU 101074
Bradyrhizobium sp. CCBAU 101075
Bradyrhizobium sp. CCBAU 101076
Bradyrhizobium sp. CCBAU 101077
Bradyrhizobium sp. CCBAU 101078
Bradyrhizobium sp. CCBAU 11315
Bradyrhizobium sp. CCBAU 11345
Bradyrhizobium sp. CCBAU 11350
Bradyrhizobium sp. CCBAU 11355
Bradyrhizobium sp. CCBAU 11357
Bradyrhizobium sp. CCBAU 11360
Bradyrhizobium sp. CCBAU 11361
Bradyrhizobium sp. CCBAU 11379
Bradyrhizobium sp. CCBAU 11386
Bradyrhizobium sp. CCBAU 11408
Bradyrhizobium sp. CCBAU 11419
Bradyrhizobium sp. CCBAU 11423
Bradyrhizobium sp. CCBAU 11427
Bradyrhizobium sp. CCBAU 11430
Bradyrhizobium sp. CCBAU 11434
Bradyrhizobium sp. CCBAU 11440
Bradyrhizobium sp. CCBAU 11442
Bradyrhizobium sp. CCBAU 11445
Bradyrhizobium sp. CCBAU 11448
Bradyrhizobium sp. CCBAU 11450
Bradyrhizobium sp. CCBAU 11451
Bradyrhizobium sp. CCBAU 11456
Bradyrhizobium sp. CCBAU 11466
Bradyrhizobium sp. CCBAU 11472
Bradyrhizobium sp. CCBAU 15354
Bradyrhizobium sp. CCBAU 15362
Bradyrhizobium sp. CCBAU 15517
Bradyrhizobium sp. CCBAU 15540
Bradyrhizobium sp. CCBAU 15543
Bradyrhizobium sp. CCBAU 15544
Bradyrhizobium sp. CCBAU 15615
Bradyrhizobium sp. CCBAU 15618
Bradyrhizobium sp. CCBAU 15635
Bradyrhizobium sp. CCBAU 15640
Bradyrhizobium sp. CCBAU 15644
Bradyrhizobium sp. CCBAU 15647
Bradyrhizobium sp. CCBAU 15653
Bradyrhizobium sp. CCBAU 15658
Bradyrhizobium sp. CCBAU 15662
Bradyrhizobium sp. CCBAU 15732
Bradyrhizobium sp. CCBAU 15766
Bradyrhizobium sp. CCBAU 15773
Bradyrhizobium sp. CCBAU 15778
Bradyrhizobium sp. CCBAU 15796
Bradyrhizobium sp. CCBAU 15824
Bradyrhizobium sp. CCBAU 15839
Bradyrhizobium sp. CCBAU 15850
Bradyrhizobium sp. CCBAU 15868
Bradyrhizobium sp. CCBAU 15888
Bradyrhizobium sp. CCBAU 15891
Bradyrhizobium sp. CCBAU 15907
Bradyrhizobium sp. CCBAU 21338
Bradyrhizobium sp. CCBAU 21342
Bradyrhizobium sp. CCBAU 21348
Bradyrhizobium sp. CCBAU 21349
Bradyrhizobium sp. CCBAU 21355
Bradyrhizobium sp. CCBAU 21356
Bradyrhizobium sp. CCBAU 21358
Bradyrhizobium sp. CCBAU 21359
Bradyrhizobium sp. CCBAU 21360
Bradyrhizobium sp. CCBAU 21362
Bradyrhizobium sp. CCBAU 21365
Bradyrhizobium sp. CCBAU 21371
Bradyrhizobium sp. CCBAU 23005
Bradyrhizobium sp. CCBAU 23011
Bradyrhizobium sp. CCBAU 23136
Bradyrhizobium sp. CCBAU 23236
Bradyrhizobium sp. CCBAU 23309
Bradyrhizobium sp. CCBAU 23332
Bradyrhizobium sp. CCBAU 23371
Bradyrhizobium sp. CCBAU 23372
Bradyrhizobium sp. CCBAU 23373
Bradyrhizobium sp. CCBAU 23374
Bradyrhizobium sp. CCBAU 23375
Bradyrhizobium sp. CCBAU 23376
Bradyrhizobium sp. CCBAU 23377
Bradyrhizobium sp. CCBAU 25021
Bradyrhizobium sp. CCBAU 25325
Bradyrhizobium sp. CCBAU 25360
Bradyrhizobium sp. CCBAU 25374
Bradyrhizobium sp. CCBAU 25430
Bradyrhizobium sp. CCBAU 25435
Bradyrhizobium sp. CCBAU 25436
Bradyrhizobium sp. CCBAU 25437
Bradyrhizobium sp. CCBAU 25466
Bradyrhizobium sp. CCBAU 25527
Bradyrhizobium sp. CCBAU 25530
Bradyrhizobium sp. CCBAU 25533
Bradyrhizobium sp. CCBAU 25534
Bradyrhizobium sp. CCBAU 25536
Bradyrhizobium sp. CCBAU 25537
Bradyrhizobium sp. CCBAU 25541
Bradyrhizobium sp. CCBAU 25544
Bradyrhizobium sp. CCBAU 25548
Bradyrhizobium sp. CCBAU 25549
Bradyrhizobium sp. CCBAU 25573
Bradyrhizobium sp. CCBAU 25625
Bradyrhizobium sp. CCBAU 25626
Bradyrhizobium sp. CCBAU 25627
Bradyrhizobium sp. CCBAU 25628
Bradyrhizobium sp. CCBAU 25629
Bradyrhizobium sp. CCBAU 25630
Bradyrhizobium sp. CCBAU 25631
Bradyrhizobium sp. CCBAU 25632
Bradyrhizobium sp. CCBAU 25633
Bradyrhizobium sp. CCBAU 25634
Bradyrhizobium sp. CCBAU 25635
Bradyrhizobium sp. CCBAU 25636
Bradyrhizobium sp. CCBAU 25637
Bradyrhizobium sp. CCBAU 25638
Bradyrhizobium sp. CCBAU 25639
Bradyrhizobium sp. CCBAU 25640
Bradyrhizobium sp. CCBAU 25641
Bradyrhizobium sp. CCBAU 25642
Bradyrhizobium sp. CCBAU 25644
Bradyrhizobium sp. CCBAU 25645
Bradyrhizobium sp. CCBAU 25646
Bradyrhizobium sp. CCBAU 25647
Bradyrhizobium sp. CCBAU 25648
Bradyrhizobium sp. CCBAU 25649
Bradyrhizobium sp. CCBAU 25650
Bradyrhizobium sp. CCBAU 25651
Bradyrhizobium sp. CCBAU 25652
Bradyrhizobium sp. CCBAU 25653
Bradyrhizobium sp. CCBAU 25654
Bradyrhizobium sp. CCBAU 33006
Bradyrhizobium sp. CCBAU 33007
Bradyrhizobium sp. CCBAU 33011
Bradyrhizobium sp. CCBAU 33012-2
Bradyrhizobium sp. CCBAU 33037
Bradyrhizobium sp. CCBAU 33039
Bradyrhizobium sp. CCBAU 33062
Bradyrhizobium sp. CCBAU 33079
Bradyrhizobium sp. CCBAU 33098
Bradyrhizobium sp. CCBAU 33099
Bradyrhizobium sp. CCBAU 33100
Bradyrhizobium sp. CCBAU 33102
Bradyrhizobium sp. CCBAU 33109
Bradyrhizobium sp. CCBAU 33118
Bradyrhizobium sp. CCBAU 33128
Bradyrhizobium sp. CCBAU 33146
Bradyrhizobium sp. CCBAU 33158
Bradyrhizobium sp. CCBAU 35157
Bradyrhizobium sp. CCBAU 35186-1
Bradyrhizobium sp. CCBAU 35272
Bradyrhizobium sp. CCBAU 41003
Bradyrhizobium sp. CCBAU 41019
Bradyrhizobium sp. CCBAU 41032
Bradyrhizobium sp. CCBAU 41071
Bradyrhizobium sp. CCBAU 41136
Bradyrhizobium sp. CCBAU 41137
Bradyrhizobium sp. CCBAU 41152
Bradyrhizobium sp. CCBAU 41314
Bradyrhizobium sp. CCBAU 43005
Bradyrhizobium sp. CCBAU 43048
Bradyrhizobium sp. CCBAU 43058
Bradyrhizobium sp. CCBAU 43078
Bradyrhizobium sp. CCBAU 43135
Bradyrhizobium sp. CCBAU 43178
Bradyrhizobium sp. CCBAU 43224
Bradyrhizobium sp. CCBAU 43257
Bradyrhizobium sp. CCBAU 43261
Bradyrhizobium sp. CCBAU 43289
Bradyrhizobium sp. CCBAU 43292
Bradyrhizobium sp. CCBAU 43297
Bradyrhizobium sp. CCBAU 43298
Bradyrhizobium sp. CCBAU 45060
Bradyrhizobium sp. CCBAU 45066
Bradyrhizobium sp. CCBAU 45281
Bradyrhizobium sp. CCBAU 45334
Bradyrhizobium sp. CCBAU 45377
Bradyrhizobium sp. CCBAU 45382
Bradyrhizobium sp. CCBAU 45384
Bradyrhizobium sp. CCBAU 45389
Bradyrhizobium sp. CCBAU 45393
Bradyrhizobium sp. CCBAU 45501
Bradyrhizobium sp. CCBAU 45502
Bradyrhizobium sp. CCBAU 45505
Bradyrhizobium sp. CCBAU 45513
Bradyrhizobium sp. CCBAU 45515
Bradyrhizobium sp. CCBAU 45539
Bradyrhizobium sp. CCBAU 51012
Bradyrhizobium sp. CCBAU 51159
Bradyrhizobium sp. CCBAU 51172
Bradyrhizobium sp. CCBAU 51377
Bradyrhizobium sp. CCBAU 51495
Bradyrhizobium sp. CCBAU 51511
Bradyrhizobium sp. CCBAU 51515
Bradyrhizobium sp. CCBAU 51522
Bradyrhizobium sp. CCBAU 51523
Bradyrhizobium sp. CCBAU 51528
Bradyrhizobium sp. CCBAU 51529
Bradyrhizobium sp. CCBAU 51555
Bradyrhizobium sp. CCBAU 51559
Bradyrhizobium sp. CCBAU 51568
Bradyrhizobium sp. CCBAU 51569
Bradyrhizobium sp. CCBAU 51578
Bradyrhizobium sp. CCBAU 51595
Bradyrhizobium sp. CCBAU 51604
Bradyrhizobium sp. CCBAU 51607
Bradyrhizobium sp. CCBAU 51617
Bradyrhizobium sp. CCBAU 51627
Bradyrhizobium sp. CCBAU 51640
Bradyrhizobium sp. CCBAU 51660
Bradyrhizobium sp. CCBAU 51663
Bradyrhizobium sp. CCBAU 51688
Bradyrhizobium sp. CCBAU 51733
Bradyrhizobium sp. CCBAU 51736
Bradyrhizobium sp. CCBAU 51745
Bradyrhizobium sp. CCBAU 51753
Bradyrhizobium sp. CCBAU 51765
Bradyrhizobium sp. CCBAU 51768
Bradyrhizobium sp. CCBAU 51776
Bradyrhizobium sp. CCBAU 51789
Bradyrhizobium sp. CCBAU 53168
Bradyrhizobium sp. CCBAU 53273
Bradyrhizobium sp. CCBAU 53276
Bradyrhizobium sp. CCBAU 53278
Bradyrhizobium sp. CCBAU 53289
Bradyrhizobium sp. CCBAU 53298
Bradyrhizobium sp. CCBAU 53299
Bradyrhizobium sp. CCBAU 53326
Bradyrhizobium sp. CCBAU 53338
Bradyrhizobium sp. CCBAU 53340
Bradyrhizobium sp. CCBAU 53351
Bradyrhizobium sp. CCBAU 53373
Bradyrhizobium sp. CCBAU 53380
Bradyrhizobium sp. CCBAU 53415
Bradyrhizobium sp. CCBAU 53421
Bradyrhizobium sp. CCBAU 61015
Bradyrhizobium sp. CCBAU 61017
Bradyrhizobium sp. CCBAU 61018
Bradyrhizobium sp. CCBAU 61022
Bradyrhizobium sp. CCBAU 61025
Bradyrhizobium sp. CCBAU 61029
Bradyrhizobium sp. CCBAU 61035
Bradyrhizobium sp. CCBAU 61046
Bradyrhizobium sp. CCBAU 61051
Bradyrhizobium sp. CCBAU 61058
Bradyrhizobium sp. CCBAU 61069
Bradyrhizobium sp. CCBAU 61071
Bradyrhizobium sp. CCBAU 61073
Bradyrhizobium sp. CCBAU 65008
Bradyrhizobium sp. CCBAU 65066
Bradyrhizobium sp. CCBAU 65718
Bradyrhizobium sp. CCBAU 65788
Bradyrhizobium sp. CCBAU 65884
Bradyrhizobium sp. CCBAU 71325
Bradyrhizobium sp. CCBAU 71331
Bradyrhizobium sp. CCBAU 71342
Bradyrhizobium sp. CCBAU 83611
Bradyrhizobium sp. CCBAU 83614
Bradyrhizobium sp. CCBAU 83623
Bradyrhizobium sp. CCBAU 83678
Bradyrhizobium sp. CCBAU 83689
Bradyrhizobium sp. CCBAU 83699
Bradyrhizobium sp. CCGB12
Bradyrhizobium sp. CCGE-LA001
Bradyrhizobium sp. CCGUVB14
Bradyrhizobium sp. CCGUVB1N3
Bradyrhizobium sp. CCGUVB23
Bradyrhizobium sp. CCGUVB4N
Bradyrhizobium sp. CCH5-F6
Bradyrhizobium sp. CK3
Bradyrhizobium sp. CNG1
Bradyrhizobium sp. CNL1
Bradyrhizobium sp. CNL2
Bradyrhizobium sp. CNL7
Bradyrhizobium sp. CNL9
Bradyrhizobium sp. CRRI-SW13
Bradyrhizobium sp. CSR10A
Bradyrhizobium sp. CW1
Bradyrhizobium sp. Cf1
Bradyrhizobium sp. Cf3.6
Bradyrhizobium sp. Cf4
Bradyrhizobium sp. Cp5.3
Bradyrhizobium sp. CpR3
Bradyrhizobium sp. CzHD
Bradyrhizobium sp. D1(2014)
Bradyrhizobium sp. DASA 68056
Bradyrhizobium sp. DFCI-1
Bradyrhizobium sp. DOA1
Bradyrhizobium sp. DOA9
Bradyrhizobium sp. DW10.1
Bradyrhizobium sp. DW12.5
Bradyrhizobium sp. DcE2.8
Bradyrhizobium sp. DcE3.5
Bradyrhizobium sp. DcE3.6
Bradyrhizobium sp. DesT10
Bradyrhizobium sp. DesT1
Bradyrhizobium sp. Dp6.2
Bradyrhizobium sp. Dp6.5
Bradyrhizobium sp. DpLT2
Bradyrhizobium sp. DrLT5
Bradyrhizobium sp. DrLT6
Bradyrhizobium sp. DrLT7
Bradyrhizobium sp. EHJO10
Bradyrhizobium sp. EHJO1a
Bradyrhizobium sp. EHJO8
Bradyrhizobium sp. EHNEHU6
Bradyrhizobium sp. EHNS18
Bradyrhizobium sp. EHNS8
Bradyrhizobium sp. EHUS6
Bradyrhizobium sp. EPxn1
Bradyrhizobium sp. ERR10
Bradyrhizobium sp. ERR4
Bradyrhizobium sp. Ec3.3
Bradyrhizobium sp. F1-1-1
Bradyrhizobium sp. F82-1-1
Bradyrhizobium sp. F83-1-3
Bradyrhizobium sp. F85-2-3
Bradyrhizobium sp. F86-1-2
Bradyrhizobium sp. F86-1-3
Bradyrhizobium sp. FVJONG14
Bradyrhizobium sp. FVMY14
Bradyrhizobium sp. FVNS22
Bradyrhizobium sp. FVNS2
Bradyrhizobium sp. FVNS4
Bradyrhizobium sp. G127
Bradyrhizobium sp. G35
Bradyrhizobium sp. GER1
Bradyrhizobium sp. GER32
Bradyrhizobium sp. GM
Bradyrhizobium sp. GV135
Bradyrhizobium sp. GV137
Bradyrhizobium sp. GZL13-3
Bradyrhizobium sp. Gha
Bradyrhizobium sp. Ghvi
Bradyrhizobium sp. H11
Bradyrhizobium sp. HM155
Bradyrhizobium sp. HO172
Bradyrhizobium sp. HO186
Bradyrhizobium sp. HO196
Bradyrhizobium sp. HO199
Bradyrhizobium sp. I71
Bradyrhizobium sp. IAR18B
Bradyrhizobium sp. ICMP 10768
Bradyrhizobium sp. ICMP 10776
Bradyrhizobium sp. ICMP 11737
Bradyrhizobium sp. ICMP 11738
Bradyrhizobium sp. ICMP 12835
Bradyrhizobium sp. ICMP 14755
Bradyrhizobium sp. ICMP 14756
Bradyrhizobium sp. ICMP 14757
Bradyrhizobium sp. ICMP 14758
Bradyrhizobium sp. ICMP 19822
Bradyrhizobium sp. ICMP 19823
Bradyrhizobium sp. ICMP 19824
Bradyrhizobium sp. ICMP 19825
Bradyrhizobium sp. ICMP 19826
Bradyrhizobium sp. ICMP 19827
Bradyrhizobium sp. ICMP 19828
Bradyrhizobium sp. ICMP 19829
Bradyrhizobium sp. ICMP 19831
Bradyrhizobium sp. ICMP 19832
Bradyrhizobium sp. ICMP 19835
Bradyrhizobium sp. ICMP 19836
Bradyrhizobium sp. ICMP 19837
Bradyrhizobium sp. ICMP 19838
Bradyrhizobium sp. ICMP 19839
Bradyrhizobium sp. ICMP 19840
Bradyrhizobium sp. ICMP 19842
Bradyrhizobium sp. ICMP 19843
Bradyrhizobium sp. ICMP 19844
Bradyrhizobium sp. ICMP 19845
Bradyrhizobium sp. ICMP 19846
Bradyrhizobium sp. ICMP 19847
Bradyrhizobium sp. ICMP 19848
Bradyrhizobium sp. ICMP 19864
Bradyrhizobium sp. ICMP 20558
Bradyrhizobium sp. ICMP 20559
Bradyrhizobium sp. ICMP 20560
Bradyrhizobium sp. ICMP 20561
Bradyrhizobium sp. ICMP 20562
Bradyrhizobium sp. ICMP 20563
Bradyrhizobium sp. ICMP 20564
Bradyrhizobium sp. ICMP 20565
Bradyrhizobium sp. ICMP 20566
Bradyrhizobium sp. ICMP 3154
Bradyrhizobium sp. ICMP 5798
Bradyrhizobium sp. ICMP 7719
Bradyrhizobium sp. ICMP 8377
Bradyrhizobium sp. INPA01-91A
Bradyrhizobium sp. INPA104A
Bradyrhizobium sp. INPA237B
Bradyrhizobium sp. IRBG 228
Bradyrhizobium sp. IRBG 230
Bradyrhizobium sp. IRBG 2
Bradyrhizobium sp. ISA0203
Bradyrhizobium sp. ISA0206
Bradyrhizobium sp. ISA0304
Bradyrhizobium sp. ISA0403
Bradyrhizobium sp. ISA0406
Bradyrhizobium sp. ISA0408
Bradyrhizobium sp. ISA0506
Bradyrhizobium sp. ISA0508
Bradyrhizobium sp. ISA0601
Bradyrhizobium sp. ISA0901
Bradyrhizobium sp. ISA1101
Bradyrhizobium sp. ISA1301
Bradyrhizobium sp. ISA1501
Bradyrhizobium sp. ISA1601
Bradyrhizobium sp. ISA1704
Bradyrhizobium sp. ISA1901
Bradyrhizobium sp. ISA1904
Bradyrhizobium sp. ISA2206
Bradyrhizobium sp. ISA2402
Bradyrhizobium sp. ISA2507
Bradyrhizobium sp. Iri 5/11
Bradyrhizobium sp. Iri 5/12
Bradyrhizobium sp. Iri 5/14
Bradyrhizobium sp. Iri 5/4
Bradyrhizobium sp. Iri 5/5
Bradyrhizobium sp. Iri 5/6
Bradyrhizobium sp. Iri 5/7
Bradyrhizobium sp. Iri 5/8
Bradyrhizobium sp. Iri 5/9
Bradyrhizobium sp. Ishi 7/11
Bradyrhizobium sp. Ishi 7/12
Bradyrhizobium sp. Ishi 7/13
Bradyrhizobium sp. Ishi 7/2
Bradyrhizobium sp. JAN13
Bradyrhizobium sp. JAN6
Bradyrhizobium sp. JC1S4
Bradyrhizobium sp. JNVU AL39
Bradyrhizobium sp. JNVU AV34
Bradyrhizobium sp. JNVU AV46
Bradyrhizobium sp. JNVU AV48
Bradyrhizobium sp. JNVU CB4
Bradyrhizobium sp. JNVU CP19
Bradyrhizobium sp. JNVU CP24
Bradyrhizobium sp. JNVU CP30
Bradyrhizobium sp. JNVU CT11
Bradyrhizobium sp. JNVU DC11
Bradyrhizobium sp. JNVU RA25
Bradyrhizobium sp. JNVU RA63
Bradyrhizobium sp. JNVU TF10
Bradyrhizobium sp. JNVU TF4
Bradyrhizobium sp. JNVU TV10
Bradyrhizobium sp. JNVU TV11
Bradyrhizobium sp. JNVU TV4
Bradyrhizobium sp. JNVU TW14
Bradyrhizobium sp. JNVU TW7
Bradyrhizobium sp. JNVU VA7
Bradyrhizobium sp. Jan10
Bradyrhizobium sp. KO11
Bradyrhizobium sp. KO13
Bradyrhizobium sp. KO14
Bradyrhizobium sp. KO17
Bradyrhizobium sp. KO19A
Bradyrhizobium sp. KO20A
Bradyrhizobium sp. KO23
Bradyrhizobium sp. KO3A
Bradyrhizobium sp. KO3B
Bradyrhizobium sp. KO3C
Bradyrhizobium sp. KO3D
Bradyrhizobium sp. KO3E
Bradyrhizobium sp. KO3F
Bradyrhizobium sp. KO3G
Bradyrhizobium sp. KO3H
Bradyrhizobium sp. KO5
Bradyrhizobium sp. LCM 3682
Bradyrhizobium sp. LCM 3683
Bradyrhizobium sp. LCT2
Bradyrhizobium sp. LMTR 3
Bradyrhizobium sp. LUG11
Bradyrhizobium sp. LULIT23
Bradyrhizobium sp. LULIT25
Bradyrhizobium sp. LUP27
Bradyrhizobium sp. LUP3
Bradyrhizobium sp. LUP4
Bradyrhizobium sp. LVM 105
Bradyrhizobium sp. LcCT4
Bradyrhizobium sp. LcCT6
Bradyrhizobium sp. LcRI3
Bradyrhizobium sp. Lei1
Bradyrhizobium sp. Leo121
Bradyrhizobium sp. Leo170
Bradyrhizobium sp. Lh10
Bradyrhizobium sp. Lh15
Bradyrhizobium sp. LmjC
Bradyrhizobium sp. Lppb2
Bradyrhizobium sp. Lppb8
Bradyrhizobium sp. Lpsp.1b
Bradyrhizobium sp. Lpsp3.4
Bradyrhizobium sp. LupCI
Bradyrhizobium sp. M10
Bradyrhizobium sp. M12
Bradyrhizobium sp. M26
Bradyrhizobium sp. M3
Bradyrhizobium sp. MAFF 210190
Bradyrhizobium sp. MAFF 210197
Bradyrhizobium sp. MAFF 210318
Bradyrhizobium sp. MM5392
Bradyrhizobium sp. MM5405
Bradyrhizobium sp. MM5621
Bradyrhizobium sp. MOS001
Bradyrhizobium sp. MOS002
Bradyrhizobium sp. MOS003
Bradyrhizobium sp. Mf253
Bradyrhizobium sp. N + ROM18
Bradyrhizobium sp. N10
Bradyrhizobium sp. NAS80.1
Bradyrhizobium sp. NAS96.2
Bradyrhizobium sp. NBAIM01
Bradyrhizobium sp. NC92
Bradyrhizobium sp. NCSU 1933
Bradyrhizobium sp. NCSU 1943
Bradyrhizobium sp. NCSU 2028
Bradyrhizobium sp. NCSU 2039
Bradyrhizobium sp. NCSU 2061
Bradyrhizobium sp. NCSU 2112
Bradyrhizobium sp. NCSU 2114
Bradyrhizobium sp. NCSU 2185
Bradyrhizobium sp. NCSU 2192
Bradyrhizobium sp. NCSU 2199
Bradyrhizobium sp. NCSU 2204
Bradyrhizobium sp. NCSU 2207
Bradyrhizobium sp. NCSU 2211
Bradyrhizobium sp. NCSU 2223
Bradyrhizobium sp. NCSU 2248
Bradyrhizobium sp. NCSU 2265
Bradyrhizobium sp. NCSU 2320
Bradyrhizobium sp. NCSU 2333
Bradyrhizobium sp. NCSU 2335
Bradyrhizobium sp. NCSU 2358
Bradyrhizobium sp. NCSU 2369
Bradyrhizobium sp. NCSU 2384
Bradyrhizobium sp. NCSU 2406
Bradyrhizobium sp. NCSU 2438
Bradyrhizobium sp. NCSU 2446
Bradyrhizobium sp. NCSU 2473
Bradyrhizobium sp. NCSU 2476
Bradyrhizobium sp. NCSU 2513
Bradyrhizobium sp. NCSU 2536
Bradyrhizobium sp. NCSU 2576
Bradyrhizobium sp. NCSU 2579
Bradyrhizobium sp. NCSU 2583
Bradyrhizobium sp. NCSU 2631
Bradyrhizobium sp. NCSU 2632
Bradyrhizobium sp. NFR13
Bradyrhizobium sp. NPK + ROM11
Bradyrhizobium sp. NPK + ROM8
Bradyrhizobium sp. NZP 2309
Bradyrhizobium sp. OK095
Bradyrhizobium sp. OM28
Bradyrhizobium sp. OM55
Bradyrhizobium sp. OO107
Bradyrhizobium sp. ORS 277
Bradyrhizobium sp. ORS 278
Bradyrhizobium sp. ORS 279
Bradyrhizobium sp. ORS 285
Bradyrhizobium sp. ORS 287
Bradyrhizobium sp. ORS 294
Bradyrhizobium sp. ORS 297
Bradyrhizobium sp. ORS 298
Bradyrhizobium sp. ORS 300
Bradyrhizobium sp. ORS 301
Bradyrhizobium sp. ORS 303
Bradyrhizobium sp. ORS 304
Bradyrhizobium sp. ORS 305
Bradyrhizobium sp. ORS 307
Bradyrhizobium sp. ORS 309
Bradyrhizobium sp. ORS 310
Bradyrhizobium sp. ORS 318
Bradyrhizobium sp. ORS 324
Bradyrhizobium sp. ORS 3257
Bradyrhizobium sp. ORS 3258
Bradyrhizobium sp. ORS 327
Bradyrhizobium sp. ORS 328
Bradyrhizobium sp. ORS 330
Bradyrhizobium sp. ORS 331
Bradyrhizobium sp. ORS 336
Bradyrhizobium sp. ORS 344
Bradyrhizobium sp. ORS 354
Bradyrhizobium sp. ORS 356
Bradyrhizobium sp. ORS 358
Bradyrhizobium sp. ORS 361
Bradyrhizobium sp. ORS 372
Bradyrhizobium sp. ORS 375
Bradyrhizobium sp. ORS 377
Bradyrhizobium sp. ORS 391
Bradyrhizobium sp. ORS 393
Bradyrhizobium sp. ORS 400
Bradyrhizobium sp. P10-5
Bradyrhizobium sp. P10-6
Bradyrhizobium sp. P11-1
Bradyrhizobium sp. P15-7
Bradyrhizobium sp. P3-24
Bradyrhizobium sp. P4-29
Bradyrhizobium sp. P4-5
Bradyrhizobium sp. P4-7
Bradyrhizobium sp. P5-26
Bradyrhizobium sp. P5-27
Bradyrhizobium sp. P5-32
Bradyrhizobium sp. P5-5
Bradyrhizobium sp. P7-17
Bradyrhizobium sp. P7-35
Bradyrhizobium sp. P7-40
Bradyrhizobium sp. P8-11
Bradyrhizobium sp. P8-5
Bradyrhizobium sp. P9-21
Bradyrhizobium sp. P9-22
Bradyrhizobium sp. PRIMUS42
Bradyrhizobium sp. PZHK10
Bradyrhizobium sp. PZHK2
Bradyrhizobium sp. PZHK6
Bradyrhizobium sp. PZHK7
Bradyrhizobium sp. PZHK8
Bradyrhizobium sp. PZHK9
Bradyrhizobium sp. R-45547
Bradyrhizobium sp. R-45579
Bradyrhizobium sp. R-45657
Bradyrhizobium sp. R-45662
Bradyrhizobium sp. R-45722
Bradyrhizobium sp. R-45743 = 1532
Bradyrhizobium sp. R-45821
Bradyrhizobium sp. R-45957
Bradyrhizobium sp. R-46016
Bradyrhizobium sp. R-46017
Bradyrhizobium sp. R-46151 = t187
Bradyrhizobium sp. R-46172 = t240
Bradyrhizobium sp. R-46173 = t242
Bradyrhizobium sp. R-46206 = t384
Bradyrhizobium sp. R-46210 = t414
Bradyrhizobium sp. R-46220
Bradyrhizobium sp. R-46224
Bradyrhizobium sp. R-46277 = t668
Bradyrhizobium sp. R-46304 = t779
Bradyrhizobium sp. R-46306 = t782
Bradyrhizobium sp. R-46310 = t789
Bradyrhizobium sp. R-46311 = t792
Bradyrhizobium sp. R-46313 = t795
Bradyrhizobium sp. R2.2-H
Bradyrhizobium sp. RITF1002
Bradyrhizobium sp. RITF1015
Bradyrhizobium sp. RITF1022
Bradyrhizobium sp. RITF1023
Bradyrhizobium sp. RITF1024
Bradyrhizobium sp. RITF1102
Bradyrhizobium sp. RITF1106
Bradyrhizobium sp. RITF1109
Bradyrhizobium sp. RITF1110
Bradyrhizobium sp. RITF1112
Bradyrhizobium sp. RITF1115
Bradyrhizobium sp. RITF1116
Bradyrhizobium sp. RITF1118
Bradyrhizobium sp. RITF11
Bradyrhizobium sp. RITF1313
Bradyrhizobium sp. RITF1315
Bradyrhizobium sp. RITF1323
Bradyrhizobium sp. RITF1328
Bradyrhizobium sp. RITF14115
Bradyrhizobium sp. RITF1412
Bradyrhizobium sp. RITF1416
Bradyrhizobium sp. RITF1417
Bradyrhizobium sp. RITF1431
Bradyrhizobium sp. RITF1432
Bradyrhizobium sp. RITF1482
Bradyrhizobium sp. RITF1541
Bradyrhizobium sp. RITF1542
Bradyrhizobium sp. RITF1551
Bradyrhizobium sp. RITF1552
Bradyrhizobium sp. RITF1556
Bradyrhizobium sp. RITF1558
Bradyrhizobium sp. RITF1612
Bradyrhizobium sp. RITF1621
Bradyrhizobium sp. RITF1622
Bradyrhizobium sp. RITF16241
Bradyrhizobium sp. RITF1624
Bradyrhizobium sp. RITF1631
Bradyrhizobium sp. RITF1632
Bradyrhizobium sp. RITF1633
Bradyrhizobium sp. RITF1653
Bradyrhizobium sp. RITF1654
Bradyrhizobium sp. RITF1656
Bradyrhizobium sp. RITF1657
Bradyrhizobium sp. RITF1658
Bradyrhizobium sp. RITF512
Bradyrhizobium sp. RITF522
Bradyrhizobium sp. RITF63
Bradyrhizobium sp. RITF651
Bradyrhizobium sp. RITF671
Bradyrhizobium sp. RITF681
Bradyrhizobium sp. RITF801
Bradyrhizobium sp. ROM2
Bradyrhizobium sp. RP6
Bradyrhizobium sp. RRD24
Bradyrhizobium sp. RRM3
Bradyrhizobium sp. RRM8
Bradyrhizobium sp. RRSSET16
Bradyrhizobium sp. RS13
Bradyrhizobium sp. RSA104
Bradyrhizobium sp. RSA3
Bradyrhizobium sp. RSB2
Bradyrhizobium sp. RSB6
Bradyrhizobium sp. RSS137
Bradyrhizobium sp. Rc2d
Bradyrhizobium sp. Rc3b
Bradyrhizobium sp. S36
Bradyrhizobium sp. S4 0
Bradyrhizobium sp. SBR1B
Bradyrhizobium sp. SBR6
Bradyrhizobium sp. SCAU211
Bradyrhizobium sp. SCAUd24
Bradyrhizobium sp. SCAUd29
Bradyrhizobium sp. SDD10-1
Bradyrhizobium sp. SDD12-1
Bradyrhizobium sp. SDD14-1
Bradyrhizobium sp. SDD21-3
Bradyrhizobium sp. SDD22-4
Bradyrhizobium sp. SDD24-1
Bradyrhizobium sp. SDD42-1
Bradyrhizobium sp. SDD43-2
Bradyrhizobium sp. SEMIA 6014
Bradyrhizobium sp. SEMIA 6028
Bradyrhizobium sp. SEMIA 6053
Bradyrhizobium sp. SEMIA 6069
Bradyrhizobium sp. SEMIA 6077
Bradyrhizobium sp. SEMIA 6093
Bradyrhizobium sp. SEMIA 6099
Bradyrhizobium sp. SEMIA 6101
Bradyrhizobium sp. SEMIA 6146
Bradyrhizobium sp. SEMIA 6152
Bradyrhizobium sp. SEMIA 6156
Bradyrhizobium sp. SEMIA 6160
Bradyrhizobium sp. SEMIA 6163
Bradyrhizobium sp. SEMIA 6164
Bradyrhizobium sp. SEMIA 6179
Bradyrhizobium sp. SEMIA 6186
Bradyrhizobium sp. SEMIA 6187
Bradyrhizobium sp. SEMIA 6192
Bradyrhizobium sp. SEMIA 6374
Bradyrhizobium sp. SEMIA 6434
Bradyrhizobium sp. SEMIA 6440
Bradyrhizobium sp. SEMIA 656
Bradyrhizobium sp. SEMIA 695
Bradyrhizobium sp. SEMIA
Bradyrhizobium sp. SHOUNA76
Bradyrhizobium sp. SR106
Bradyrhizobium sp. SR66
Bradyrhizobium sp. SR69
Bradyrhizobium sp. SR78
Bradyrhizobium sp. SRS-191
Bradyrhizobium sp. STM 3040
Bradyrhizobium sp. STM 3043
Bradyrhizobium sp. STM 3047
Bradyrhizobium sp. STM 3049
Bradyrhizobium sp. STM 3059
Bradyrhizobium sp. STM 3060
Bradyrhizobium sp. STM 3062
Bradyrhizobium sp. STM 3068
Bradyrhizobium sp. STM 3070
Bradyrhizobium sp. STM 3074
Bradyrhizobium sp. STM 3078
Bradyrhizobium sp. STM 3080
Bradyrhizobium sp. STM 3082
Bradyrhizobium sp. STM 3088
Bradyrhizobium sp. STM 3090
Bradyrhizobium sp. STM 3091
Bradyrhizobium sp. STM 3809
Bradyrhizobium sp. STM 3843
Bradyrhizobium sp. STM 7332
Bradyrhizobium sp. STM 7333
Bradyrhizobium sp. STM 7334
Bradyrhizobium sp. STM 7378
Bradyrhizobium sp. STM 8076
Bradyrhizobium sp. STM 8087
Bradyrhizobium sp. STM 8099
Bradyrhizobium sp. SUTN9-2
Bradyrhizobium sp. SZCZ11
Bradyrhizobium sp. SZCZ20
Bradyrhizobium sp. SZCZ6
Bradyrhizobium sp. TSA15y
Bradyrhizobium sp. TSA26
Bradyrhizobium sp. TSA27b
Bradyrhizobium sp. TSA27s
Bradyrhizobium sp. TSA43
Bradyrhizobium sp. TSA44
Bradyrhizobium sp. TUTAHSA10
Bradyrhizobium sp. TUTAHSA115
Bradyrhizobium sp. TUTAHSA118
Bradyrhizobium sp. TUTAHSA126
Bradyrhizobium sp. TUTAHSA155
Bradyrhizobium sp. TUTAHSA27
Bradyrhizobium sp. TUTAHSA31
Bradyrhizobium sp. TUTAHSA40
Bradyrhizobium sp. TUTAHSA4
Bradyrhizobium sp. TUTAHSA67
Bradyrhizobium sp. TUTAHSA75
Bradyrhizobium sp. TUTAHSA7
Bradyrhizobium sp. TUTAHSA80
Bradyrhizobium sp. TUTAHSA87
Bradyrhizobium sp. TUTCG2.I
Bradyrhizobium sp. TUTCG4.I
Bradyrhizobium sp. TUTCM1C
Bradyrhizobium sp. TUTCS2.I
Bradyrhizobium sp. TUTCSA1A
Bradyrhizobium sp. TUTCSA1B
Bradyrhizobium sp. TUTCSA2.I
Bradyrhizobium sp. TUTCSA2C
Bradyrhizobium sp. TUTCSA3A
Bradyrhizobium sp. TUTCSA3B
Bradyrhizobium sp. TUTCSA3C
Bradyrhizobium sp. TUTCSA4.I
Bradyrhizobium sp. TUTCSA4A
Bradyrhizobium sp. TUTCSA4B
Bradyrhizobium sp. TUTCSA4C
Bradyrhizobium sp. TUTCSA5A
Bradyrhizobium sp. TUTCT1.I
Bradyrhizobium sp. TUTCT2A
Bradyrhizobium sp. TUTCT3A
Bradyrhizobium sp. TUTCT4A
Bradyrhizobium sp. TUTDAIAP1A
Bradyrhizobium sp. TUTDAIAP2A1
Bradyrhizobium sp. TUTDAIAP3B
Bradyrhizobium sp. TUTDAIAP8B
Bradyrhizobium sp. TUTGP10.10.2
Bradyrhizobium sp. TUTLl2.10.7
Bradyrhizobium sp. TUTLl2.20.6
Bradyrhizobium sp. TUTLl2.20.7
Bradyrhizobium sp. TUTLl4.10.5b
Bradyrhizobium sp. TUTLl4.10.7
Bradyrhizobium sp. TUTLl4.10.9
Bradyrhizobium sp. TUTLl7.10.3
Bradyrhizobium sp. TUTLl8.20.5
Bradyrhizobium sp. TUTMCJ4B
Bradyrhizobium sp. TUTMCJ5B
Bradyrhizobium sp. TUTMCJ7B
Bradyrhizobium sp. TUTMCJ9A
Bradyrhizobium sp. TUTMFJ1A1
Bradyrhizobium sp. TUTMFJ2B
Bradyrhizobium sp. TUTMIITA5A1
Bradyrhizobium sp. TUTMIITA5A2
Bradyrhizobium sp. TUTMP10.6
Bradyrhizobium sp. TUTMP12.13
Bradyrhizobium sp. TUTMP14.10.3b
Bradyrhizobium sp. TUTMP16.11
Bradyrhizobium sp. TUTMP18.11
Bradyrhizobium sp. TUTMP18.6
Bradyrhizobium sp. TUTMP19.10
Bradyrhizobium sp. TUTMP2.9
Bradyrhizobium sp. TUTMP7.7
Bradyrhizobium sp. TUTMP9.7
Bradyrhizobium sp. TUTN17405
Bradyrhizobium sp. TUTNFM2A1
Bradyrhizobium sp. TUTRAB5B1
Bradyrhizobium sp. TUTRAH5B2
Bradyrhizobium sp. TUTRLR3B
Bradyrhizobium sp. TUTVSBEG
Bradyrhizobium sp. TUTVSBEK
Bradyrhizobium sp. TUTVSBEM
Bradyrhizobium sp. TUTVSBESA
Bradyrhizobium sp. TUTVSBLG
Bradyrhizobium sp. TUTVSBLK
Bradyrhizobium sp. TUTVSBMK
Bradyrhizobium sp. TUTVSBMSA
Bradyrhizobium sp. TUTVSCRG
Bradyrhizobium sp. TUTVSCRK
Bradyrhizobium sp. TUTVSCRM-I
Bradyrhizobium sp. TUTVSRDK-I
Bradyrhizobium sp. TUTVSRDK
Bradyrhizobium sp. TUTVSRDM
Bradyrhizobium sp. TUTVSRDS
Bradyrhizobium sp. TUTVSRMK
Bradyrhizobium sp. TUTVSRMSA
Bradyrhizobium sp. TUTVSRMT
Bradyrhizobium sp. Tep17
Bradyrhizobium sp. Tep1
Bradyrhizobium sp. Tep5
Bradyrhizobium sp. Tv2a-2
Bradyrhizobium sp. UFLA 03-321
Bradyrhizobium sp. UFLA03-144
Bradyrhizobium sp. UFLA03-153
Bradyrhizobium sp. UFLA03-268
Bradyrhizobium sp. UFLA03-84
Bradyrhizobium sp. UFLA06-05
Bradyrhizobium sp. UFLA06-06
Bradyrhizobium sp. UFLA06-10
Bradyrhizobium sp. UFLA06-13
Bradyrhizobium sp. UFLA06-15
Bradyrhizobium sp. UFLA06-19
Bradyrhizobium sp. UFLA06-21
Bradyrhizobium sp. UFLA06-22
Bradyrhizobium sp. UNPA324
Bradyrhizobium sp. UNPF46
Bradyrhizobium sp. URHA0013
Bradyrhizobium sp. USDA 4541
Bradyrhizobium sp. VUCR24
Bradyrhizobium sp. VUEP21
Bradyrhizobium sp. VULI11
Bradyrhizobium sp. VULI21
Bradyrhizobium sp. WBAH10
Bradyrhizobium sp. WBAH11
Bradyrhizobium sp. WBAH23
Bradyrhizobium sp. WBAH24
Bradyrhizobium sp. WBAH25
Bradyrhizobium sp. WBAH27
Bradyrhizobium sp. WBAH29
Bradyrhizobium sp. WBAH30
Bradyrhizobium sp. WBAH31
Bradyrhizobium sp. WBAH33
Bradyrhizobium sp. WBAH35
Bradyrhizobium sp. WBAH38
Bradyrhizobium sp. WBAH39
Bradyrhizobium sp. WBAH40
Bradyrhizobium sp. WBAH41
Bradyrhizobium sp. WBAH42
Bradyrhizobium sp. WBAH6
Bradyrhizobium sp. WBOS10
Bradyrhizobium sp. WBOS12
Bradyrhizobium sp. WBOS13
Bradyrhizobium sp. WBOS16
Bradyrhizobium sp. WBOS18
Bradyrhizobium sp. WBOS1
Bradyrhizobium sp. WBOS2
Bradyrhizobium sp. WBOS3
Bradyrhizobium sp. WBOS4
Bradyrhizobium sp. WBOS5
Bradyrhizobium sp. WBOS6
Bradyrhizobium sp. WBOS7
Bradyrhizobium sp. WBOS8
Bradyrhizobium sp. WD16
Bradyrhizobium sp. WSM1253
Bradyrhizobium sp. WSM1417
Bradyrhizobium sp. WSM2254
Bradyrhizobium sp. WSM3983
Bradyrhizobium sp. WSM471
Bradyrhizobium sp. WYCCWR 12678
Bradyrhizobium sp. WYCCWR 12774
Bradyrhizobium sp. WYCCWR 13023
Bradyrhizobium sp. Y-H1
Bradyrhizobium sp. YIC60756
Bradyrhizobium sp. YIC60767
Bradyrhizobium sp. YIC60838
Bradyrhizobium sp. YIC60933
Bradyrhizobium sp. YIC60953
Bradyrhizobium sp. YIC61000
Bradyrhizobium sp. YIC61059
Bradyrhizobium sp. Z15
Bradyrhizobium sp. ZAR12
Bradyrhizobium sp. ZAR2
Bradyrhizobium sp. ZAR4
Bradyrhizobium sp. ZAR7
Bradyrhizobium sp. aai25
Bradyrhizobium sp. aai2
Bradyrhizobium sp. abh10
Bradyrhizobium sp. abh7
Bradyrhizobium sp. abs7
Bradyrhizobium sp. abw9
Bradyrhizobium sp. bth12
Bradyrhizobium sp. bth1
Bradyrhizobium sp. cf659
Bradyrhizobium sp. cfhr.1a
Bradyrhizobium sp. cfrr1
Bradyrhizobium sp. cfs4
Bradyrhizobium sp. cfs5
Bradyrhizobium sp. cmh1
Bradyrhizobium sp. cmw2
Bradyrhizobium sp. cmy11
Bradyrhizobium sp. cmy17
Bradyrhizobium sp. cna3
Bradyrhizobium sp. cnw15
Bradyrhizobium sp. cscc5
Bradyrhizobium sp. csds5
Bradyrhizobium sp. csjp11
Bradyrhizobium sp. cslt5
Bradyrhizobium sp. csma27
Bradyrhizobium sp. csmx9
Bradyrhizobium sp. cspf11
Bradyrhizobium sp. csri27
Bradyrhizobium sp. cvg10
Bradyrhizobium sp. cvg25
Bradyrhizobium sp. cvr1
Bradyrhizobium sp. dnr7
Bradyrhizobium sp. dpg38
Bradyrhizobium sp. dpg39
Bradyrhizobium sp. dps3
Bradyrhizobium sp. dw_78
Bradyrhizobium sp. gvg6
Bradyrhizobium sp. gvr5
Bradyrhizobium sp. gvw6
Bradyrhizobium sp. lia4
Bradyrhizobium sp. ll4
Bradyrhizobium sp. lrs5
Bradyrhizobium sp. lrw2
Bradyrhizobium sp. onl92.10
Bradyrhizobium sp. onl92.6
Bradyrhizobium sp. rhw4
Bradyrhizobium sp. rhyd.1a
Bradyrhizobium sp. rp3
Bradyrhizobium sp. sba8
Bradyrhizobium sp. sbw22
Bradyrhizobium sp. sbw3
Bradyrhizobium sp. sbw5
Bradyrhizobium sp. teph5
Bradyrhizobium sp. teva11
Bradyrhizobium sp. teva23
Bradyrhizobium sp. th.b2
Bradyrhizobium sp. zb15
Bradyrhizobium sp. zb1
Bradyrhizobium sp. zb34
Bradyrhizobium sp. zb36
Bradyrhizobium sp.
Bradyrhizobium stylosanthis
Bradyrhizobium subterraneum
Bradyrhizobium symbiodeficiens
Bradyrhizobium tropiciagri
Bradyrhizobium uaiense
Bradyrhizobium valentinum
Bradyrhizobium vignae
Bradyrhizobium viridifuturi
Bradyrhizobium yuanmingense CCBAU 05623
Bradyrhizobium yuanmingense
Bradyrhizobium zhanjiangense
Bradyrhizobium
Branchiibius hedensis
Brasilonema bromeliae
Brasilonema octagenarum UFV-E1
Brasilonema octagenarum
Brasilonema sennae CENA114
Brasilonema sennae
Brasilonema sp. CENA360
Brasilonema sp. CENA361
Brasilonema sp. CENA366
Brasilonema sp. CENA381
Brasilonema sp. CENA382
Brasilonema sp. CT11
Brasilonema sp. NQAIF325
Brasilonema sp. UFV-LI
Brasilonema
Brassica napus
Bremerella cremea
Brenneria alni
Brenneria goodwinii
Brenneria izadpanahii
Brenneria nigrifluens
Brenneria roseae subsp. americana
Brenneria roseae subsp. roseae
Brenneria roseae
Brenneria rubrifaciens
Brenneria salicis ATCC 15712 = DSM 30166
Brenneria salicis
Brenneria sp. 4F2
Brenneria sp. CFCC 11842
Brenneria sp. EniD312
Brenneria sp. L3-3C-1
Brenneria sp. hezel4-2-4
Brenneria tiliae
Brenneria
Brevibacillus agri
Brevibacillus borstelensis AK1
Brevibacillus borstelensis cifa_chp40
Brevibacillus borstelensis
Brevibacillus choshinensis
Brevibacillus laterosporus GI-9
Brevibacillus laterosporus
Brevibacillus massiliensis
Brevibacillus panacihumi W25
Brevibacillus panacihumi
Brevibacillus sp. BC25
Brevibacillus sp. WF146
Brevibacterium antiquum
Brevibacterium atlanticum
Brevibacterium aurantiacum
Brevibacterium casei
Brevibacterium epidermidis
Brevibacterium iodinum
Brevibacterium limosum
Brevibacterium linens
Brevibacterium marinum
Brevibacterium mcbrellneri
Brevibacterium oceani
Brevibacterium permense
Brevibacterium pigmentatum
Brevibacterium renqingii
Brevibacterium sandarakinum
Brevibacterium sediminis
Brevibacterium siliguriense
Brevibacterium sp. ‘Marine’
Brevibacterium sp. 239c
Brevibacterium sp. BDJS002
Brevibacterium sp. CCUG 69071
Brevibacterium sp. CFH 10365
Brevibacterium sp. CT2-23B
Brevibacterium sp. FME17
Brevibacterium sp. FME37
Brevibacterium sp. JSBI002
Brevibacterium sp. R8603A2
Brevibacterium sp. RIT 803
Brevibacterium sp. S111
Brevibacterium sp. S22
Brevibacterium sp. SMBL_HHYL_HB1
Brevibacterium sp. UCMA 11752
Brevibacterium sp. UCMA 11754
Brevibacterium sp. VCM10
Brevibacterium sp. WHS-Z9
Brevibacterium sp. ZH18
Brevibacterium yomogidense
Brevibacterium zhoupengii
Brevibacterium
Brevundimonas basaltis
Brevundimonas diminuta
Brevundimonas fluminis
Brevundimonas intermedia
Brevundimonas sp. 12-68-7
Brevundimonas sp. 32-68-21
Brevundimonas sp. EYE_349
Brevundimonas sp. LM2
Brevundimonas sp. M20
Brevundimonas sp. TN37
Brevundimonas sp. TN40
Brevundimonas sp. TN44
Brevundimonas sp.
Brevundimonas subvibrioides ATCC 15264
Brevundimonas subvibrioides
Brevundimonas viscosa
Breznakibacter xylanolyticus
Breznakiella homolactica
Brooklawnia cerclae
Brucella anthropi
Brucella cytisi
Brucella intermedia
Brucella lupini
Brucella melitensis
Brucella ovis
Brucella pinnipedialis
Brucella sp. 191011898
Brucella suis
Brucella tritici
Burkholderia aenigmatica
Burkholderia ambifaria IOP40-10
Burkholderia ambifaria
Burkholderia anthina
Burkholderia arboris
Burkholderia cenocepacia BC7
Burkholderia cenocepacia
Burkholderia cepacia
Burkholderia contaminans
Burkholderia diffusa
Burkholderia gladioli
Burkholderia glumae
Burkholderia lata
Burkholderia latens
Burkholderia metallica
Burkholderia multivorans ATCC BAA-247
Burkholderia multivorans CGD2M
Burkholderia multivorans CGD2
Burkholderia multivorans
Burkholderia oklahomensis
Burkholderia plantarii
Burkholderia pseudomallei A79A
Burkholderia pseudomallei A79C
Burkholderia pseudomallei A79D
Burkholderia pseudomallei B03
Burkholderia pseudomalleiMSHR332
Burkholderia pseudomallei MSHR3335
Burkholderia pseudomallei MSHR4300
Burkholderia pseudomallei MSHR4303
Burkholderia pseudomallei MSHR435
Burkholderia pseudomallei MSHR7498
Burkholderia pseudomallei MSHR449
Burkholderia pseudomallei MSHR491
Burkholderia pseudomallei MSHR6137
Burkholderia pseudomallei MSHR4378
Burkholderia pseudomallei TSV 31
Burkholderia pseudomallei TSV 43
Burkholderia pseudomallei
Burkholderia pseudomultivorans
Burkholderia pyrrocinia
Burkholderia singularis
Burkholderia sp. 7016
Burkholderia sp. A2
Burkholderia sp. AU15512
Burkholderia sp. AU16482
Burkholderia sp. AU16741
Burkholderia sp. AU17457
Burkholderia sp. AU18528
Burkholderia sp. AU27893
Burkholderia sp. AU28863
Burkholderia sp. AU33647
Burkholderia sp. AU33803
Burkholderia sp. AU4i
Burkholderia sp. Ac-20344
Burkholderia sp. Ac-20345
Burkholderia sp. Ac-20349
Burkholderia sp. Ac-20353
Burkholderia sp. Ac-20365
Burkholderia sp. Ac-20379
Burkholderia sp. Ac-20392
Burkholderia sp. Ap-962
Burkholderia sp. Ax-1719
Burkholderia sp. Ax-1724
Burkholderia sp. BL10 I2R1
Burkholderia sp. BL13-R2
Burkholderia sp. BL15 R3
Burkholderia sp. BL16 I4R2
Burkholderia sp. BL16 R3
Burkholderia sp. BL17 R1
Burkholderia sp. BL18 I3R2
Burkholderia sp. BL18 I8R1
Burkholderia sp. BL18 I8R3
Burkholderia sp. BL21 I4R1
Burkholderia sp. BL21 15R3
Burkholderia sp. BL23 I1R1
Burkholderia sp. BL23 I2R2
Burkholderia sp. BL25 I1R1
Burkholderia sp. BL25 I3R1
Burkholderia sp. BL25 I3R6
Burkholderia sp. BL27 I3R6
Burkholderia sp. BL27 I4R3
Burkholderia sp. BL29 I6R2
Burkholderia sp. BL6665C I2R2
Burkholderia sp. BL9 I2R2
Burkholderia sp. BRUESC13
Burkholderia sp. BRUESC14
Burkholderia sp. BRUESC20
Burkholderia sp. BRUESC34
Burkholderia sp. BRUESC862
Burkholderia sp. Br3469
Burkholderia sp. CB2
Burkholderia sp. CI1
Burkholderia sp. CI2
Burkholderia sp. CI3
Burkholderia sp. CRRI-76_SBUA
Burkholderia sp. CS2
Burkholderia sp. Ch1-1
Burkholderia sp. Clong1
Burkholderia sp. Clong3
Burkholderia sp. Cs130
Burkholderia sp. Cses4
Burkholderia sp. Cy-647
Burkholderia sp. D1SS235
Burkholderia sp. D7
Burkholderia sp. DUS751
Burkholderia sp. Gc145
Burkholderia sp. HAN2018
Burkholderia sp. HC1.1a1
Burkholderia sp. HC1.1bd
Burkholderia sp. HI2761
Burkholderia sp. HMSC10F09
Burkholderia sp. ICMP 19548
Burkholderia sp. ICMP 19549
Burkholderia sp. ICMP 19866
Burkholderia sp. ICMP 19867
Burkholderia sp. ICMP 19869
Burkholderia sp. IIIA_4A
Burkholderia sp. JP2-270
Burkholderia sp. JPY105
Burkholderia sp. JPY155
Burkholderia sp. JPY157
Burkholderia sp. JPY159
Burkholderia sp. JPY160
Burkholderia sp. JPY164
Burkholderia sp. JPY166
Burkholderia sp. JPY167
Burkholderia sp. JPY168
Burkholderia sp. JPY170
Burkholderia sp. JPY209
Burkholderia sp. JPY255
Burkholderia sp. JPY256
Burkholderia sp. JPY257
Burkholderia sp. JPY258
Burkholderia sp. JPY259
Burkholderia sp. JPY262
Burkholderia sp. JPY264
Burkholderia sp. JPY266
Burkholderia sp. JPY268
Burkholderia sp. JPY270
Burkholderia sp. JPY271
Burkholderia sp. JPY272
Burkholderia sp. JPY276
Burkholderia sp. JPY277
Burkholderia sp. JPY278
Burkholderia sp. JPY279
Burkholderia sp. JPY280
Burkholderia sp. JPY281
Burkholderia sp. JPY282
Burkholderia sp. JPY283
Burkholderia sp. JPY285
Burkholderia sp. JPY286
Burkholderia sp. JPY288
Burkholderia sp. JPY294
Burkholderia sp. JPY296
Burkholderia sp. JPY298
Burkholderia sp. JPY300
Burkholderia sp. JPY301
Burkholderia sp. JPY307
Burkholderia sp. JPY309
Burkholderia sp. JPY310
Burkholderia sp. JPY312
Burkholderia sp. JPY314
Burkholderia sp. JPY315
Burkholderia sp. JPY317
Burkholderia sp. JPY321
Burkholderia sp. JPY323
Burkholderia sp. JPY324
Burkholderia sp. JPY325
Burkholderia sp. JPY326
Burkholderia sp. JPY345
Burkholderia sp. JPY346
Burkholderia sp. JPY349
Burkholderia sp. JPY350
Burkholderia sp. JPY351
Burkholderia sp. JPY366
Burkholderia sp. JPY368
Burkholderia sp. JPY380
Burkholderia sp. JPY381
Burkholderia sp. JPY382
Burkholderia sp. JPY384
Burkholderia sp. JPY385
Burkholderia sp. JPY386
Burkholderia sp. JPY388
Burkholderia sp. JPY406
Burkholderia sp. JPY409
Burkholderia sp. JPY410
Burkholderia sp. JPY411
Burkholderia sp. JPY413
Burkholderia sp. JPY414
Burkholderia sp. JPY417
Burkholderia sp. JPY420
Burkholderia sp. JPY430
Burkholderia sp. JPY431
Burkholderia sp. JPY434
Burkholderia sp. JPY439
Burkholderia sp. JPY441
Burkholderia sp. JPY449
Burkholderia sp. JPY452
Burkholderia sp. JPY453
Burkholderia sp. JPY465
Burkholderia sp. JPY467
Burkholderia sp. JPY471
Burkholderia sp. JPY472
Burkholderia sp. JPY476
Burkholderia sp. JPY478
Burkholderia sp. JPY479
Burkholderia sp. JPY481
Burkholderia sp. JPY484
Burkholderia sp. JPY487
Burkholderia sp. JPY490
Burkholderia sp. JPY492
Burkholderia sp. JPY504
Burkholderia sp. JPY578
Burkholderia sp. JPY581
Burkholderia sp. JPY582
Burkholderia sp. JPY584
Burkholderia sp. JPY585
Burkholderia sp. JPY587
Burkholderia sp. JPY595
Burkholderia sp. JPY604
Burkholderia sp. JPY605
Burkholderia sp. JPY629
Burkholderia sp. JPY634
Burkholderia sp. JPY636
Burkholderia sp. JPY637
Burkholderia sp. JPY680
Burkholderia sp. JPY682
Burkholderia sp. JPY687
Burkholderia sp. JPY690
Burkholderia sp. JPY694
Burkholderia sp. JPY696
Burkholderia sp. JPY697
Burkholderia sp. JPY802
Burkholderia sp. JPY804
Burkholderia sp. JPY807
Burkholderia sp. KJ006
Burkholderia sp. Kb6
Burkholderia sp. LK4
Burkholderia sp. LMG 13014
Burkholderia sp. Leaf177
Burkholderia sp. MBR-1
Burkholderia sp. MM5337
Burkholderia sp. MM5366
Burkholderia sp. MM5384
Burkholderia sp. MM5477
Burkholderia sp. MM5482
Burkholderia sp. MM5496
Burkholderia sp. MM5746
Burkholderia sp. MM5875
Burkholderia sp. MM5878
Burkholderia sp. MPB11
Burkholderia sp. MPB1
Burkholderia sp. MPB6
Burkholderia sp. MPB8
Burkholderia sp. Ms116
Burkholderia sp. NGR190
Burkholderia sp. OD120
Burkholderia sp. OD28
Burkholderia sp. OD55
Burkholderia sp. PAMC 26561
Burkholderia sp. PAMC 28687
Burkholderia sp. PTK47
Burkholderia sp. Pgonoacantha_8
Burkholderia sp. RAU2c
Burkholderia sp. RAU2d
Burkholderia sp. RAU2f
Burkholderia sp. RAU2g
Burkholderia sp. RAU2i
Burkholderia sp. RAU2k
Burkholderia sp. RAU2l
Burkholderia sp. RAU6.4a
Burkholderia sp. RAU6.4b
Burkholderia sp. S171
Burkholderia sp. SCN-KJ
Burkholderia sp. SG-MS1
Burkholderia sp. SMF 774_1
Burkholderia sp. STM 7292
Burkholderia sp. STM 7317
Burkholderia sp. STM 7324
Burkholderia sp. STM 7415
Burkholderia sp. STM 7437
Burkholderia sp. STM 7454
Burkholderia sp. SWF66044
Burkholderia sp. SWF67297
Burkholderia sp. Se-20378
Burkholderia sp. TJ182
Burkholderia sp. Tr-862
Burkholderia sp. UCT15
Burkholderia sp. UCT30
Burkholderia sp. UCT34
Burkholderia sp. UCT43
Burkholderia sp. UCT56
Burkholderia sp. UCT70
Burkholderia sp. UCT71
Burkholderia sp. UYPR1.313
Burkholderia sp. UYPR1.3
Burkholderia sp. UYPR1.413
Burkholderia sp. UYPR1.45
Burkholderia sp. UYPR2.522
Burkholderia sp. UYPR3.611
Burkholderia sp. UYPR4.732
Burkholderia sp. UYPR5.94
Burkholderia sp. UYPR6.101
Burkholderia sp. WC7.3a
Burkholderia sp. WC7.3c
Burkholderia sp. WC7.3f
Burkholderia sp. WK.1k
Burkholderia sp. WK1.1e
Burkholderia sp. WP9
Burkholderia sp. WSM2232
Burkholderia sp. YR290
Burkholderia sp. enrichment culture clone CACua-01
Burkholderia sp. enrichment culture clone CACua-09
Burkholderia sp. enrichment culture clone CACua-11
Burkholderia sp. enrichment culture clone CACua-34
Burkholderia sp. enrichment culture clone CACua-50
Burkholderia sp. enrichment culture clone CACua-70
Burkholderia sp. enrichment culture clone CACua-88
Burkholderia sp. enrichment culture clone CATux-28
Burkholderia sp. enrichment culture clone CATux-299
Burkholderia sp. enrichment culture clone CATux-328
Burkholderia sp. enrichment culture clone CATux-331
Burkholderia sp. enrichment culture clone CATux-40
Burkholderia sp. enrichment culture clone CATux-4
Burkholderia sp. enrichment culture clone CATux-90
Burkholderia sp. enrichment culture clone CATux-95
Burkholderia sp. gx97
Burkholderia sp. lig30
Burkholderia sp. mpig8.9
Burkholderia sp. mpp4
Burkholderia sp. mpp5
Burkholderia sp. mpud4.5
Burkholderia sp. mpud5.2
Burkholderia sp.
Burkholderia stabilis
Burkholderia stagnalis
Burkholderia territorii
Burkholderia thailandensis
Burkholderia ubonensis MSMB22
Burkholderia ubonensis subsp. mesacidophila
Burkholderia ubonensis
Burkholderia vietnamiensis G4
Burkholderia vietnamiensis LMG 10929
Burkholderia vietnamiensis
Burkholderia
Burkholderiaceae bacterium
Burkholderiaceae
Burkholderiales bacterium 28-67-8
Burkholderiales bacterium GJ-E10
Burkholderiales bacterium GWE1_65_30
Burkholderiales bacterium GWF1_66_17
Burkholderiales bacterium JOSHI_001
Burkholderiales bacterium PBB1
Burkholderiales bacterium PBB2
Burkholderiales bacterium PBB5
Burkholderiales bacterium PBB6
Burkholderiales bacterium RIFCSPHIGHO2_12_FULL_67_38
Burkholderiales bacterium RIFCSPLOWO2_02_FULL_67_64
Burkholderiales bacterium RIFCSPLOWO2_12_67_14
Burkholderiales bacterium
Butyrivibrio crossotus CAG: 259
Butyrivibrio crossotus DSM 2876
Butyrivibrio crossotus
Butyrivibrio fibrisolvens 16/4
Butyrivibrio fibrisolvens DSM 3071
Butyrivibrio fibrisolvens
Butyrivibrio hungatei DSM 14810
Butyrivibrio hungatei
Butyrivibrio proteoclasticus B316
Butyrivibrio proteoclasticus
Butyrivibrio sp. AC2005
Butyrivibrio sp. AE2015
Butyrivibrio sp. AE2032
Butyrivibrio sp. AE3004
Butyrivibrio sp. CAG: 318
Butyrivibrio sp. DSM 10294
Butyrivibrio sp. FCS014
Butyrivibrio sp. INlla16
Butyrivibrio sp. INlla21
Butyrivibrio sp. LB2008
Butyrivibrio sp. M55
Butyrivibrio sp. MC2021
Butyrivibrio sp. NC2002
Butyrivibrio sp. NC2007
Butyrivibrio sp. NC3005
Butyrivibrio sp. TB
Butyrivibrio sp. VCB2006
Butyrivibrio sp. VCD2006
Butyrivibrio sp. WCD3002
Butyrivibrio sp. X503
Butyrivibrio sp. XB500-5
Butyrivibrio sp. XPD2006
Butyrivibrio sp. YAB3001
Butyrivibrio sp. ob235
Butyrivibrio
Caballeronia arvi
Caballeronia calidae
Caballeronia catudaia
Caballeronia concitans
Caballeronia cordobensis
Caballeronia fortuita
Caballeronia grimmiae
Caballeronia hypogeia
Caballeronia mineralivorans
Caballeronia novacaledonica
Caballeronia pedi
Caballeronia ptereochthonis
Caballeronia sordidicola
Caballeronia sp. ATUFL_M2_KS44
Caballeronia sp. GACF4
Caballeronia sp. GAFFF1
Caballeronia sp. GAFFF2
Caballeronia sp. GAFFF3
Caballeronia sp. GAWG1-1
Caballeronia sp. GAWG2-1
Caballeronia sp. INDeC2
Caballeronia sp. INSB1
Caballeronia sp. Lep1P3
Caballeronia sp. TF1N1
Caballeronia sp. dw_19
Caballeronia sp. dw_276
Caballeronia telluris
Caballeronia temeraria
Caballeronia udeis
Caballeronia zhejiangensis
Caballeronia
Caenimonas koreensis
Caenimonas sp. HX-9-20
Caenimonas sp. SL110
Caenispirillum bisanense
Caenispirillum salinarum AK4
Caenispirillum salinarum
Caenorhabditis briggsae
Cajanus cajan
Caldalkalibacillus mannanilyticus
Caldalkalibacillus thermarum TA2.A1
Caldalkalibacillus thermarum
Caldanaerobacter subterraneus subsp. yonseiensis KB-1
Caldanaerobacter subterraneus
Caldanaerobius fijiensis
Caldanaerobius polysaccharolyticus
Calderihabitans maritimus
Caldibacillus hisashii
Caldibacillus sp. 210928-DFI.2.18
Caldibacillus sp. 210928-DFI.2.22
Caldibacillus thermoamylovorans
Caldicellulosiruptor acetigenus 6A
Caldicellulosiruptor acetigenus I77R1B
Caldicellulosiruptor acetigenus
Caldicellulosiruptor bescii
Caldicellulosiruptor danielii
Caldicellulosiruptor diazotrophicus
Caldicellulosiruptor hydrothermalis 108
Caldicellulosiruptor hydrothermalis
Caldicellulosiruptor kronotskyensis 2002
Caldicellulosiruptor kronotskyensis
Caldicellulosiruptor morganii
Caldicellulosiruptor naganoensis
Caldicellulosiruptor obsidiansis OB47
Caldicellulosiruptor obsidiansis
Caldicellulosiruptor owensensis OL
Caldicellulosiruptor owensensis
Caldicellulosiruptor saccharolyticus DSM 8903
Caldicellulosiruptor saccharolyticus
Caldicellulosiruptor sp. F32
Caldicellulosiruptor
Caldichromatium japonicum
Caldicoprobacter faecalis
Caldicoprobacter oshimai
Caldilineae bacterium
Caldimicrobium thiodismutans
Caldimonas brevitalea
Caldimonas caldifontis
Caldimonas tepidiphila
Caldinitratiruptor microaerophilus
Caldisalinibacter kiritimatiensis
Caldiserica bacterium CG02_land_8_20_14_3_00_36_38
Caldiserica bacterium CG_4_8_14_3_um_filter_35_18
Caldisericum sp. CG2_30_36_11
Calditerricola satsumensis
Calditerrivibrio nitroreducens DSM 19672
Calditerrivibrio nitroreducens
Caldithrix abyssi DSM 13497
Caldithrix abyssi
Calditrichaeota bacterium
Calenema singularis LEGE 06188
Calidifontibacter indicus
Calidithermus terrae
Callorhinus ursinus
Caloramator australicus RC3
Caloramator australicus
Caloramator mitchellensis
Caloramator sp. ALD01
Caloranaerobacter sp. TR13
Calothrix brevissima Ind10
Calothrix brevissima Ind9
Calothrix brevissima NIES-22
Calothrix brevissima
Calothrix desertica PCC 7102
Calothrix elsteri CCALA 953
Calothrix elsteri
Calothrix membranacea
Calothrix parasitica NIES-267
Calothrix parasitica
Calothrix rhizosoleniae M15
Calothrix rhizosoleniae
Calothrix sp. 336/3
Calothrix sp. 5325T1
Calothrix sp. FACHB-156
Calothrix sp. HK-06
Calothrix sp. Ind11
Calothrix sp. LCRNK_13
Calothrix sp. LCRNK_14
Calothrix sp. LCRNK_15
Calothrix sp. LEGE 06100
Calothrix sp. LEGE 06122
Calothrix sp. LEGE 07177
Calothrix sp. MCC-3A
Calothrix sp. MN-7C
Calothrix sp. NIES-2098
Calothrix sp. NIES-2100
Calothrix sp. NIES-3974
Calothrix sp. NIES-4071
Calothrix sp. NIES-4105
Calothrix sp. NQAIF310
Calothrix sp. PCC 6303
Calothrix sp. PCC 7103
Calothrix sp. PCC 7507
Calothrix sp. PCC 7716
Calothrix sp. UAM 315
Calothrix sp. UAM 370
Calothrix
Campylobacter armoricus
Campylobacter blaseri
Campylobacter coli LMG 23341
Campylobacter coli
Campylobacter concisus 13826
Campylobacter concisus
Campylobacter corcagiensis
Campylobacter curvus 525.92
Campylobacter curvus
Campylobacter fetus subsp. fetus 04/554
Campylobacter fetus subsp. fetus
Campylobacter fetus subsp. venerealis NCTC 10354
Campylobacter geochelonis
Campylobacter hyointestinalis subsp. hyointestinalis
Campylobacter hyointestinalis subsp. lawsonii CCUG 27631
Campylobacter hyointestinalis subsp. lawsonii
Campylobacter jejuni CVM 41927
Campylobacter jejuni CVM 41973
Campylobacter jejuni RM1221
Campylobacter jejuni subsp. jejuni ICDCCJ07002
Campylobacter jejuni subsp. jejuni ICDCCJ07004
Campylobacter jejuni subsp. jejuni
Campylobacter jejuni
Campylobacter lanienae NCTC 13004
Campylobacter lanienae
Campylobacter lari subsp. concheus
Campylobacter lari subsp. lari
Campylobacter mucosalis CCUG 21559
Campylobacter mucosalis
Campylobacter novaezeelandiae
Campylobacter ornithocola
Campylobacter pinnipediorum subsp. pinnipediorum
Campylobacter rectus
Campylobacter showae CC57C
Campylobacter showae CSUNSWCD
Campylobacter showae
Campylobacter sp. CCUG 57310
Campylobacter sp. P0021
Campylobacter sp. P0023
Campylobacter sp. P0024
Campylobacter sp. P0094
Campylobacter sp. P0106
Campylobacter sp. P0108
Campylobacter sp. P0124
Campylobacter sp. P0132
Campylobacter sp. P0135
Campylobacter sp. P0138
Campylobacter sp. P0139
Campylobacter sp. P0187
Campylobacter sp. P0209
Campylobacter sp. P031
Campylobacter sp. P091
Campylobacter sp. P093
Campylobacter sp. P146
Campylobacter sp. P148
Campylobacter sp. P155
Campylobacter sp. P160
Campylobacter sp. RM12175
Campylobacter sp. RM16187
Campylobacter sp. RM16192
Campylobacter sp. RM6137
Campylobacter sp. RM8964
Campylobacter sp. RM8965
Campylobacter sp. RM8966
Campylobacter sp. RM8970
Campylobacter sp. S0112
Campylobacter sp.
Campylobacter sputorum subsp. sputorum
Campylobacter ureolyticus
Campylobacter volucris
Campylobacter vulpis
Campylobacter
Campylobacteraceae bacterium 4484_4
Campylobacterales bacterium 16-40-21
Candidatus Accumulibacter abiogenesis
Candidatus Accumulibacter cognatus
Candidatus Accumulibacter contiguus
Candidatus Accumulibacter phosphatis
Candidatus Accumulibacter similis
Candidatus Accumulibacter
Candidatus Acetothermia bacterium
Candidatus Acididesulfobacter diazotrophicus
Candidatus Acididesulfobacter guangdongensis
Candidatus Acidulodesulfobacterium acidiphilum
Candidatus Acidulodesulfobacterium ferriphilum
Candidatus Adiutrix intracellularis
Candidatus Altiarchaeales archaeon WOR_SM1_SCG
Candidatus Altiarchaeales archaeon
Candidatus Altiarchaeum sp. CG03_land_8_20_14_0_80_32_61_8
Candidatus Altiarchaeum sp. CG12_big_fil_rev_8_21_14_0_65_33_22
Candidatus Altiarchaeum sp. CG2_30_32_3053
Candidatus Altiarchaeum sp. CG_4_10_14_0_8_um_filter_32_8_51
Candidatus Altiarchaeum sp. CG_4_8_14_3_um_filter_33_2054
Candidatus Altiarchaeum sp. CG_4_9_14_0_8_um_filter_32_206
Candidatus Aminicenantes bacterium RBG_13_59_9
Candidatus Aminicenantes bacterium RBG_13_62_12
Candidatus Aminicenantes bacterium RBG_16_63_16
Candidatus Aminicenantes bacterium
Candidatus Argoarchaeum ethanivorans
Candidatus Atelocyanobacterium thalassa SAG_AD-638_J10
Candidatus Atelocyanobacterium thalassa isolate ALOHA
Candidatus Atelocyanobacterium thalassa isolate SIO0111C06A_T7
Candidatus Atelocyanobacterium thalassa isolate SIO0111C09A_T7
Candidatus Atelocyanobacterium thalassa isolate SIO0111C10A_T7
Candidatus Atelocyanobacterium thalassa isolate SIO0111C12A_T7
Candidatus Atelocyanobacterium thalassa isolate SIO0511E01A_T7
Candidatus Atelocyanobacterium thalassa isolate SIO0910A05A_T7
Candidatus Atelocyanobacterium thalassa isolate SIO1210B03A_T7
Candidatus Atelocyanobacterium thalassa isolate SIO1210B04A_T7
Candidatus Atelocyanobacterium thalassa isolate SIO1210B08A_T7
Candidatus Atelocyanobacterium thalassa isolate SIO64986
Candidatus Atelocyanobacterium thalassa
Candidatus Atribacteria bacterium HGW-Atribacteria-1
Candidatus Azobacteroides pseudotrichonymphae genomovar. CFP2
Candidatus Azobacteroides pseudotrichonymphae
Candidatus Bacteroides intestinigallinarum
Candidatus Bathyarchaeota archaeon B24
Candidatus Bathyarchaeota archaeon B25
Candidatus Bathyarchaeota archaeon B26-2
Candidatus Bathyarchaeota archaeon B63
Candidatus Bathyarchaeota archaeon CG07_land_8_20_14_0_80_47_9
Candidatus Bathyarchaeota archaeon RBG_16_48_13
Candidatus Bathyarchaeota archaeon ex4484_135
Candidatus Bathyarchaeota archaeon ex4484_218
Candidatus Bathyarchaeota archaeon
Candidatus Brocadia fulgida
Candidatus Brocadia sinica
Candidatus Campylobacter infans
Candidatus Carbobacillus altaicus
Candidatus Carsonella ruddii CE isolate Thao2000
Candidatus Carsonella ruddii CS isolate Thao2000
Candidatus Carsonella ruddii
Candidatus Chloroploca asiatica
Candidatus Chloroploca sp. Khr17
Candidatus Chloroploca sp. M50-1
Candidatus Cloacimonas sp. SDB
Candidatus Cloacimonetes bacterium HGW-Cloacimonetes-2
Candidatus Cloacimonetes bacterium HGW-Cloacimonetes-3
Candidatus Cloacimonetes bacterium
Candidatus Competibacter denitrificans Run_A_D11
Candidatus Competibacter denitrificans
Candidatus Competibacter phosphatis
Candidatus Competibacteraceae bacterium
Candidatus Contendobacter odensis Run_B_J11
Candidatus Contendobacter odensis
Candidatus Contendobacter sp.
Candidatus Contubernalis alkalaceticum
Candidatus Dactylopiibacterium carminicum
Candidatus Dadabacteria bacterium
Candidatus Defluviicoccus seviourii
Candidatus Dehalogenimonas etheniformans
Candidatus Desantisbacteria bacterium CG02_land_8_20_14_3_00_49_13
Candidatus Desantisbacteria bacterium CG23 combo_of_CG06-09_8_20_14_all_40_23
Candidatus Desantisbacteria bacterium CG_4_10_14_0_8_um_filter_48_2_2
Candidatus Desantisbacteria bacterium CG_4_10_14_3_um_filter_40_18
Candidatus Desulfofervidus auxilii
Candidatus Desulforudis audaxviator MP 104C
Candidatus Desulforudis audaxviator
Candidatus Desulforudis sp.
Candidatus Desulfosporosinus infrequens
Candidatus Electronema sp. GS
Candidatus Electrothrix aarhusiensis
Candidatus Endolissoclinum sp. TMED26
Candidatus Endolissoclinum sp. TMED37
Candidatus Endolissoclinum sp. TMED55
Candidatus Endoriftia persephone
Candidatus Enterovibrio escacola
Candidatus Entotheonella factor
Candidatus Entotheonella palauensis
Candidatus Entotheonella serta
Candidatus Erwinia dacicola
Candidatus Falkowbacteria bacterium CG10_big_fil_rev_8_21_14_0_10_39_9
Candidatus Fermentibacter daniensis
Candidatus Fermentibacteria bacterium
Candidatus Filomicrobium marinum
Candidatus Fluviicola riflensis
Candidatus Formimonas warabiya
Candidatus Frackibacter sp. T328-2
Candidatus Frankia alpina
Candidatus Frankia californiensis
Candidatus Frankia datiscae
Candidatus Frankia nodulisporulans
Candidatus Galacturonibacter soehngenii
Candidatus Gastranaerophilales bacterium HUM_10
Candidatus Gastranaerophilales bacterium HUM_11
Candidatus Gastranaerophilales bacterium HUM_13
Candidatus Gastranaerophilales bacterium HUM_15
Candidatus Gastranaerophilales bacterium HUM_16
Candidatus Gastranaerophilales bacterium HUM_17
Candidatus Gastranaerophilales bacterium HUM_18
Candidatus Gastranaerophilales bacterium HUM_19
Candidatus Gastranaerophilales bacterium HUM_22
Candidatus Gastranaerophilales bacterium HUM_23
Candidatus Gastranaerophilales bacterium HUM_3
Candidatus Gastranaerophilales bacterium HUM_4
Candidatus Gastranaerophilales bacterium HUM_5
Candidatus Gastranaerophilales bacterium HUM_8
Candidatus Geothermarchaeota archaeon
Candidatus Heimdallarchaeota archaeon AB_125
Candidatus Heimdallarchaeota archaeon B3_Heim
Candidatus Heimdallarchaeota archaeon LC_2
Candidatus Heimdallarchaeota archaeon
Candidatus Hydrogenedentes bacterium
Candidatus Hydrothermarchaeota archaeon
Candidatus Izimaplasma bacterium HR1
Candidatus Izimaplasma bacterium HR2
Candidatus Kentron sp. FW
Candidatus Kerfeldbacteria bacterium CG15_BIG_FIL_POST_REV_8_21_14_020_45_12
Candidatus Kerfeldbacteria bacterium CG_4_9_14_3_um_filter_45_8
Candidatus Korarchaeota archaeon
Candidatus Koribacter versatilis
Candidatus Kryptobacter tengchongensis
Candidatus Kryptonium thompsoni
Candidatus Kuenenia stuttgartiensis
Candidatus Laterigemmans baculatus
Candidatus Latescibacteria bacterium
Candidatus Lokiarchaeota archaeon CR_4
Candidatus Lokiarchaeota archaeon
Candidatus Macondimonas diazotrophica
Candidatus Magasanikbacteria bacterium CG1_02_32_51
Candidatus Magasanikbacteria bacterium CG_1_10_14_0_8_um_filter_32_14
Candidatus magnetaquicoccus inordinatus
Candidatus Magnetobacterium bavaricum
Candidatus Magnetobacterium casensis
Candidatus Magnetococcus massalia
Candidatus Magnetoglobus milticellularis str. Araruama
Candidatus Magnetominusculus xianensis
Candidatus Magnetomorum sp. HK-1
Candidatus Magnetoovum chiemensis
Candidatus malacoplasma girerdii
Candidatus Margulisbacteria bacterium GWD2_39_127
Candidatus Margulisbacteria bacterium GWE2_39_32
Candidatus Margulisbacteria bacterium GWF2_38_17
Candidatus Margulisbacteria bacterium
Candidatus Marinarcus aquaticus
Candidatus Marinimicrobia bacterium
Candidatus Marsarchaeota G1 archaeon BE_D
Candidatus Marsarchaeota G1 archaeon OSP_C
Candidatus Marsarchaeota G1 archaeon OSP_D
Candidatus Methanarcanum hacksteinii
Candidatus Methanofastidiosum methylthiophilus
Candidatus Merhanogranum sp. U3.2.1
Candidatus Methanoalarchaeum thermophilum
Candidatus Methanoliparum sp. LAM-1
Candidatus Methanoliparum thermophilum
Candidatus Methanolliviera hydrocarbonicum
Candidatus Methanolliviera sp. GoM_asphalt
Candidatus Methanolliviera sp. GoM_oil
Candidatus Methanomassiliicoccus intestinalis Issoire-Mx1
Candidatus Methanomassiliicoccus intestinalis
Candidatus Methanomethylophilus alvus Mx1201
Candidatus Methanomethylophilus alvus
Candidatus Methanomethylophilus sp. 1R26
Candidatus Methanoperedenaceae archaeron GB37
Candidatus Methanoperedenaceae archaeron GB50
Candidatus Methanoperedenaceae archaeron
Candidatus Methanoperedens nitroreducens
Candidatus Methanoperedens sp. BLZ1
Candidatus Methanoperedens sp. BLZ2
Candidatus Methanoperedens sp.
Candidatus Methanoplasma termitum
Candidatus Methanosuratincola subterraneus
Candidatus Methylobacter favarea
Candidatus Methylobacter oryzae
Candidatus Methylospirs mobilis
Candidatus Methylumidiphilus alinenensis
Candidatus Micropelagos thuwalensis
Candidatus Moduliflexus flocculans
Candidatus Mycobacterium methanotrophicum
Candidatus Mycolicibacterium alkanivorans
Candidatus Nitrospira inopinata
Candidatus Nitrospira nitrificans
Candidatus Nitrospira nitrosa
Candidatus Nitrospira sp. CP45
Candidatus Nitrospira sp. LAW
Candidatus Nitrospira sp. MKT
Candidatus Odinarchaeum yellowstonii
Candidatus Omnitrophica bacterium 4484_49
Candidatus Omnitrophica bacterium CG11_big_fil_rev_8_21_14_0_20_45_26
Candidatus Omnitrophica bacterium CG12_big_fil_rev_8_21_14_0_65_45_16
Candidatus Omnitrophica bacterium
Candidatus Ordinivivax streblomastigis
Candidatus Oscillochloris fontis
Candidatus Pacebacteria bacterium CG10_big_fil_rev_8_21_14_0_10_45_6
Candidatus Paracaedimonas acanthamoebae
Candidatus Phaeomarinobacter ectocarpi
Candidatus Photodesmus blepharus
Candidatus Photodesmus katoptron Akat1
Candidatus Photodesmus katoptron
Candidatus Phycosocius bacilliformis
Candidatus Portnoybacteria bacterium CG10_big_fil_rev_8_21_14_0_10_36_7
Candidatus Promineofilum breve
Candidatus Propionivibrio aalborgensis
Candidatus Proteinoplasmatales archaeon SG8-5
Candidatus Rhodoblastus alkanivorans
Candidatus Riflebacteria bacterium HGW-Riflebacteria-1
Candidatus Roizmanbacteria bacterium CG22_combo_CG10-13_8_21_14_all_33_16
Candidatus Roizmanbacteria bacterium CG2_30_33_16
Candidatus Roizmanbacteria bacterium CG_4_10_14_3_um_filter_33_21
Candidatus Roizmanbacteria bacterium CG_4_9_14_0_8_um_filter_34_12
Candidatus Rokubacteria bacterium
Candidatus Saccharibacteria bacterium SW_7_54_9
Candidatus Schekmanbacteria bacterium
Candidatus Sedimenticola endophacoides
Candidatus Sulfuricurvum sp. RIFRC-1
Candidatus Sulfurimonas baltica
Candidatus Sulfurimonas marisnigri
Candidatus Symbiobacter mobilis CR
Candidatus Symbiobacter mobilis
Candidatus Symbiopectobacterium endolongispinus
Candidatus Symbiopectobacterium sp. ‘North America’
Candidatus Symbiopectobacterium sp. NZEC135
Candidatus Syntrophoarchaeum sp. GoM_oil
Candidatus Syntrophoarchaeum sp. WYZ-LMO15
Candidatus Tenderia electrophaga
Candidatus Terasakiella magnetica
Candidatus Thermofonsia Clade 1 bacterium
Candidatus Thiodiazotropha endoloripes
Candidatus Thiodiazotropha endolucinida
Candidatus Thiodictyon syntrophicum
Candidatus Thiomargarita nelsonii
Candidatus Thiosymbion oneisti
Candidatus Thiothrix singaporensis
Candidatus Thorarchaeota archaeon AB_25
Candidatus Thorarchaeota archaeon SMTZ-45
Candidatus Thorarchaeota archaeon SMTZ1-45
Candidatus Thorarchaeota archaeon SMTZ1-83
Candidatus Thorarchaeota archaeon
Candidatus Vecturithrix granuli
Candidatus Viadribacter manganicus
Candidatus Viridilinea halotolerans
Candidatus Viridilinea mediisalina
Candidatus Wallbacteria bacterium HGW-Wallbacteria-1
Candidatus Wolframiiraptor sp.
Candidimonas bauzanensis
Capillimicrobium parvum
Capnocytophaga canimorsus Cc5
Capnocytophaga canimorsus
Caproicibacter fermentans
Caproicibacterium amylolyticum
Caproicibacterium lactatifermentans
Caproiciproducens sp. NJN-50
Capsicum baccatum
Carboxydocella sp. JDF658
Carboxydocella sp. ULO1
Carboxydocella sporoproducens DSM 16521
Carboxydocella thermautotrophica
Carboxydocella
Carboxydothermus ferrireducens
Carboxydothermus hydrogenoformans Z-2901
Carboxydothermus hydrogenoformans
Carboxydothermus islandicus
Carboxydothermus pertinax
Carboxylicivirga linearis
Carboxylicivirga sp. A043
Cardiobacterium valvarum
Carica papaya
Castellaniella defragrans
Castellaniella sp. S9
Catalinimonas alkaloidigena
Catellatospora citrea
Catenisphaera adipataccumulans
Catenovulum agarivorans
Catenovulum maritimum
Catenulispora pinisilvae
Catenulispora pinistramenti
Catenulispora rubra
Catenuloplanes japonicus
Catonella morbi ATCC 51271
Catonella morbi
Caulobacter endophyticus
Caulobacter flavus
Caulobacter mirabilis
Caulobacter phage CcrColossus
Caulobacter radicis
Caulobacter segnis
Caulobacter sp. 602-1
Caulobacter sp. 602-2
Caulobacter sp. CCH9-E1
Caulobacter sp. FWC2
Caulobacter sp. HMWF009
Caulobacter sp. HMWF025
Caulobacter sp. RHG1
Caulobacter sp. UNC358MFTsu5.1
Caulobacter vibrioides
Caulobacter zeae
Caulobacterales bacterium 32-69-10
Caulochytrium protostelioides
Celeribacter ethanolicus
Celeribacter halophilus
Celeribacter indicus
Celeribacter marinus
Celeribacter persicus
Celerinatantimonas diazotrophica
Celerinatantimonas yamalensis
Cellulomonas biazotea
Cellulomonas bogoriensis
Cellulomonas cellasea
Cellulomonas chitinilytica
Cellulomonas fengjieae
Cellulomonas fimi ATCC 484
Cellulomonas flavigena
Cellulomonas fulva
Cellulomonas gelida
Cellulomonas gilvus
Cellulomonas humilata
Cellulomonas iranensis
Cellulomonas massiliensis
Cellulomonas oligotrophica
Cellulomonas palmilytica
Cellulomonas phragmiteti
Cellulomonas shaoxiangyii
Cellulomonas sp. A375-1
Cellulomonas sp. B6
Cellulomonas sp. HLT2-17
Cellulomonas sp. JH27-2
Cellulomonas sp. KH9
Cellulomonas sp. KRMCY2
Cellulomonas sp. Leaf334
Cellulomonas sp. Leaf395
Cellulomonas sp. P24
Cellulomonas sp. Root137
Cellulomonas sp. Root485
Cellulomonas sp. S1-8
Cellulomonas sp. SLBN-39
Cellulomonas sp. URHD0024
Cellulomonas sp. WB94
Cellulomonas terrae
Cellulomonas xylanilytica
Cellulomonas
Cellulophaga baltica
Cellulosilyticum lentocellum DSM 5427
Cellulosilyticum lentocellum
Cellulosilyticum ruminicola
Cellulosilyticum sp. I15G1012
Cellulosilyticum sp. WCF-2
Cellulosimicrobium aquatile
Cellulosimicrobium cellulans
Cellulosimicrobium funkei
Cellulosimicrobium marinum
Cellulosimicrobium sp. 72-3
Cellulosimicrobium sp. CUA-896
Cellulosimicrobium sp. KWT-B
Cellulosimicrobium sp. MM
Cellulosimicrobium sp. SH8
Cellulosimicrobium sp. SL-1
Cellulosimicrobium sp. TH-20
Cellulosimicrobium
Cellvibrio japonicus Ueda107
Cellvibrio mixtus
Cellvibrio sp. BR
Cellvibrio sp. OA-2007
Cellvibrio sp. pealriver
Cellvibrio zantedeschiae
Cellvibrionaceae bacterium Bs08
Cellvibrionales bacterium TMED 157
Cellvibrionales bacterium TMED79
Centipeda periodontii DSM 2778
Centipeda periodontii
Cereibacter azotoformans
Cereibacter changlensis JA139
Cereibacter changlensis
Cereibacter johrii
Cereibacter ovatus
Cereibacter sphaeroides 2.4.1
Cereibacter sphaeroides KD131
Cereibacter sphaeroides WS8N
Cereibacter sphaeroides f. sp. denitrificans
Cereibacter sphaeroides
Cereibacter
Cesiribacter andamanensis
Cesiribacter sp. SM1
Chamaesiphon minutus PCC 6605
Chamaesiphon minutus
Chamaesiphon polymorphus CCALA 037
Chamaesiphon polymorphus
Chelativorans sp. ZYF759
Chelatococcus asaccharovorans
Chelatococcus sp. HY11
Chelatococcus sp. YT9
Chenggangzhangella methanolivorans
Chiayiivirga flava
Chitinibacter sp. GC72
Chitinibacter sp. ZOR0017
Chitinibacter tainanensis
Chitinimonas arctica
Chitinimonas sp. BJYL2
Chitiniphilus eburneus
Chitiniphilus shinanonensis
Chitiniphilus sp. CD1
Chitinispirillum alkaliphilum
Chitinivorax sp. B
Chitinolyticbacter sp. YIM B06366
Chitinophaga agri
Chitinophaga agrisoli
Chitinophaga arvensicola
Chitinophaga pinensis
Chitinophaga rhizophila
Chitinophaga rupis
Chitinophaga sancti
Chitinophaga silvisoli
Chitinophaga sp. GbtcB8
Chitinophaga sp. R8
Chitinophaga sp. S165
Chitinophaga sp. YR627
Chitinophaga terrae (ex Kim and Jung 2007)
Chlamydiae bacterium SM23_39
Chlorobaculum limnaeum
Chlorobaculum macestae
Chlorobaculum parvum NCIB 8327
Chlorobaculum parvum
Chlorobaculum sp. 24CR
Chlorobaculum sp. MV4-Y
Chlorobaculum tepidum TLS
Chlorobaculum tepidum
Chlorobaculum thiosulfatiphilum
Chlorobium ferrooxidans DSM 13031
Chlorobium limicola DSM 245
Chlorobium limicola
Chlorobium phaeobacteroides DSM 266
Chlorobium phaeobacteroides
Chlorobium phaeovibrioides
Chlorobium sp. KB01
Chlorobium sp. N1
Chlorobium sp.
Chlorobium
Chloroflexi bacterium B3_Chlor
Chloroflexi bacterium HGW-Chloroflexi-10
Chloroflexi bacterium HGW-Chloroflexi-3
Chloroflexi bacterium HGW-Chloroflexi-4
Chloroflexi bacterium HGW-Chloroflexi-5
Chloroflexi bacterium HGW-Chloroflexi-6
Chloroflexi bacterium HGW-Chloroflexi-8
Chloroflexi bacterium RBG_13_46_9
Chloroflexi bacterium RBG_16_56_11
Chloroflexia bacterium SDU3-3
Chloroflexota bacterium
Chloroflexus aggregans DSM 9485
Chloroflexus aggregans
Chloroflexus aurantiacus J-10-fl
Chloroflexus aurantiacus
Chloroflexus sp. Y-396-1
Chlorogloea sp. CCALA 695
Chlorogloeopsis fritschii PCC 6912
Chlorogloeopsis fritschii
Chloroherpeton thalassium ATCC 35110
Chloroherpeton thalassium
Chondrocystis sp. NIES-4102
Chondromyces apiculatus
Chondromyces crocatus
Christensenella hongkongensis
Christensenella minuta
Christensenella sp. NSJ-35
Chromatiaceae bacterium
Chromatiales bacterium
Chromatium okenii
Chromatium weissei
Chromobacterium alticapitis
Chromobacterium amazonense
Chromobacterium haemolyticum
Chromobacterium phragmitis
Chromobacterium piscinae
Chromobacterium sinusclupearum
Chromobacterium sp. ASV5
Chromobacterium sp. ATCC 53434
Chromobacterium sp. F49
Chromobacterium sp. LK11
Chromobacterium sp. LK1
Chromobacterium subtsugae
Chromobacterium vaccinii
Chromobacterium violaceum
Chromobacterium
Chroococcidiopsis cubana CCALA 043
Chroococcidiopsis cubana SAG 39.79
Chroococcidiopsis cubana
Chroococcidiopsis sp. CCALA 051
Chroococcidiopsis sp. CCMEE 29
Chroococcidiopsis sp. CCNUC1
Chroococcidiopsis sp. FACHB-1243
Chroococcidiopsis sp. MMG-5
Chroococcidiopsis sp. MMG-6
Chroococcidiopsis sp. TS-821
Chroococcidiopsis thermalis PCC 7203
Chroococcidiopsis thermalis
Chroococcidiopsis
Chroococcus sp. FPU101
Chroogloeocystis siderophila
Chryseobacterium aahli
Chryseobacterium angstadtii
Chryseobacterium antibioticum
Chryseobacterium arachidis
Chryseobacterium arthrosphaerae
Chryseobacterium aurantiacum
Chryseobacterium aureum
Chryseobacterium balustinum
Chryseobacterium binzhouense
Chryseobacterium camelliae
Chryseobacterium carnipullorum
Chryseobacterium cheonjiense
Chryseobacterium culicis
Chryseobacterium daeguense
Chryseobacterium echinoideorum
Chryseobacterium endalhagicum
Chryseobacterium faecale
Chryseobacterium fistulae
Chryseobacterium flavum
Chryseobacterium foetidum
Chryseobacterium gambrini
Chryseobacterium ginsenosidimutans
Chryseobacterium gleum
Chryseobacterium gregarium
Chryseobacterium group
Chryseobacterium gwangjuense
Chryseobacterium hagamense
Chryseobacterium hispalense
Chryseobacterium indologenes
Chryseobacterium indoltheticum
Chryseobacterium jejuense
Chryseobacterium lactis
Chryseobacterium lacus
Chryseobacterium luteum
Chryseobacterium mucoviscidosis
Chryseobacterium muglaense
Chryseobacterium mulctrae
Chryseobacterium nakagawai
Chryseobacterium nematophagum
Chryseobacterium nepalense
Chryseobacterium oranimense
Chryseobacterium oryzae
Chryseobacterium panacisoli
Chryseobacterium paridis
Chryseobacterium pennipullorum
Chryseobacterium phosphatilyticum
Chryseobacterium piscicola
Chryseobacterium populi
Chryseobacterium potabilaquae
Chryseobacterium rhizoplanae
Chryseobacterium schmidteae
Chryseobacterium sediminis
Chryseobacterium shigense
Chryseobacterium soli
Chryseobacterium sp. 18068
Chryseobacterium sp. 2VB
Chryseobacterium sp. 3008163
Chryseobacterium sp. 52
Chryseobacterium sp. 5_R23647
Chryseobacterium sp. CCBAU 10985
Chryseobacterium sp. EO14
Chryseobacterium sp. ERMR1: 04
Chryseobacterium sp. FH1
Chryseobacterium sp. G0186
Chryseobacterium sp. G0240
Chryseobacterium sp. GCR5
Chryseobacterium sp. HSC-36S06
Chryseobacterium sp. IHB B 17019
Chryseobacterium sp. JUb7
Chryseobacterium sp. KBW03
Chryseobacterium sp. LC2016-29
Chryseobacterium sp. Leaf180
Chryseobacterium sp. MEBOG06
Chryseobacterium sp. MEBOG07
Chryseobacterium sp. MFBS3-17
Chryseobacterium sp. MMS21-Ot14
Chryseobacterium sp. PC2-12
Chryseobacterium sp. PET-29
Chryseobacterium sp. POL2
Chryseobacterium sp. RR2-3-20
Chryseobacterium sp. SC28
Chryseobacterium sp. SN22
Chryseobacterium sp. SSA4.19
Chryseobacterium sp. StRB126
Chryseobacterium sp. T16E-39
Chryseobacterium sp. X308
Chryseobacterium sp. YIM B08800
Chryseobacterium sp. c4a
Chryseobacterium sp.
Chryseobacterium taeanense
Chryseobacterium tagetis
Chryseobacterium taichungense
Chryseobacterium taihuense
Chryseobacterium taiwanense
Chryseobacterium takakiae
Chryseobacterium turcicum
Chryseobacterium viscerum
Chryseobacterium vrystaatense
Chryseobacterium wanjuense
Chryseobacterium
Chryseoglobus frigidaquae
Chryseoglobus indicus
Chryseoglobus sp. 28M-23
Chryseolinea sp. Jin1
Chryseosolibacter indicus
Chryseotalea sanaruensis
Chrysiogenes arsenatis
Chthoniobacterales bacterium
Chthonobacter rhizosphaerae
Cicer arietinum
Ciceribacter ferrooxidans
Ciceribacter lividus
Ciceribacter
Circular ssDNA virus sp.
Citreicoccus inhibens
Citreimonas salinaria
Citricoccus sp. CH26A
Citricoccus sp. NR2
Citricoccus sp. SGAir0253
Citricoccus zhacaiensis
Citrifermentans bemidjiense Bem
Citrifermentans bemidjiense
Citrifermentans bremense
Citrifermentans pelophilum
Citrifermentans
Citrobacter freundii
Citrobacter sp. BDA59-3
Citrobacter sp. MNAZ 1397
Citrobacter sp. TBCS-14
Citrobacter sp. wls826
Citrobacter werkmanii
Citromicrobium bathyomarinum
Citromicrobium sp. JL1351
Citromicrobium sp. JL2201
Citromicrobium sp. JL31
Citromicrobium sp. JL477
Citromicrobium sp. JLT1363
Citromicrobium sp. RCC1897
Citromicrobium sp. WPS32
Citromicrobium
Citrus clementina
Citrus sinensis
Citrus unshiu
Clavibacter michiganensis subsp. michiganensis NCPPB 382
Clavibacter michiganensis
Cloacibacillus evryensis DSM 19522
Cloacibacillus evryensis
Cloacibacillus porcorum
Cloacibacterium rupense
Cloning vector SBa_000534
Cloning vector pCV_cistron14
Cloning vector pCV_cistron34
Cloning vector pMON261406
Clostridia bacterium 41_269
Clostridia bacterium 62_21
Clostridia bacterium BRH_c25
Clostridia bacterium
Clostridia
Clostridiaceae bacterium AF18-31LB
Clostridiaceae bacterium AF29-16BH
Clostridiaceae bacterium AF31-3BH
Clostridiaceae bacterium AF42-6
Clostridiaceae bacterium AM27-36LB
Clostridiaceae bacterium BL-3
Clostridiaceae bacterium BRH_c20a
Clostridiaceae bacterium JG1575
Clostridiaceae bacterium Marseille-Q4143
Clostridiaceae bacterium OF09-1
Clostridiaceae bacterium OM08-6BH
Clostridiales bacterium 38_11
Clostridiales bacterium 41_21_two_genomes
Clostridiales bacterium 44_9
Clostridiales bacterium AM23-16LB
Clostridiales bacterium CHKCI001
Clostridiales bacterium PH28_bin88
Clostridiales bacterium TF09-2AC
Clostridiales bacterium oral taxon 876 str. F0540
Clostridiaceae bacterium
Clostridiisalibacter paucivorans
Clostridioides difficile CD144
Clostridioides difficile CD160
Clostridioides difficile CD21
Clostridioides difficile DA00196
Clostridioides difficile DA00215
Clostridioides difficile E10
Clostridioides difficile E1
Clostridioides difficile F314
Clostridioides difficile M120
Clostridioides difficile NAP07
Clostridioides difficile NAP08
Clostridioides difficile P50
Clostridioides difficile T17
Clostridioides difficile T20
Clostridioides difficile T5
Clostridioides difficile Y270
Clostridioides difficile Y343
Clostridioides difficile
Clostridioides mangenotii
Clostridioides sp. ES-S-0010-02
Clostridioides sp. ES-S-0173-01
Clostridioides sp. ES-W-0016-02
Clostridioides sp. ZZV14-6387
Clostridioides sp. ZZV15-6598
Clostridium aceticum
Clostridium acetobutylicum ATCC 824
Clostridium acetobutylicum DSM 1731
Clostridium acetobutylicum EA 2018
Clostridium acetobutylicum
Clostridium acidisoli DSM 12555
Clostridium acidisoli
Clostridium akagii
Clostridium algidicarnis
Clostridium algifaecis
Clostridium algoriphilum
Clostridium amazonitimonense
Clostridium aminobutyricum
Clostridium arbusti
Clostridium argentinense CDC 2741
Clostridium argentinense
Clostridium autoethanogenum DSM 10061
Clostridium autoethanogenum
Clostridium baratii
Clostridium bartlettii CAG: 1329
Clostridium beijerinckii ATCC 35702
Clostridium beijerinckii NCIMB 8052
Clostridium beijerinckii NRRL B-598
Clostridium beijerinckii
Clostridium bornimense
Clostridium botulinum 202F
Clostridium botulinum A str. ATCC 19397
Clostridium botulinum A str. ATCC 3502
Clostridium botulinum A str. Hall
Clostridium botulinum A1 str. CFSAN002368
Clostridium botulinum A2 117
Clostridium botulinum A2 str. Kyoto
Clostridium botulinum A2B3 87
Clostridium botulinum A2B7 92
Clostridium botulinum A3 str. Loch Maree
Clostridium botulinum Af84
Clostridium botulinum B str. Eklund 17B (NRP)
Clostridium botulinum B str. Osaka05
Clostridium botulinum B1 str. Okra
Clostridium botulinum B2 128
Clostridium botulinum B2 267
Clostridium botulinum B2 275
Clostridium botulinum B2 331
Clostridium botulinum B2 433
Clostridium botulinum B2 450
Clostridium botulinum BKT015925
Clostridium botulinum Ba4 str. 657
Clostridium botulinum Bf
Clostridium botulinum C str. Eklund
Clostridium botulinum C/D str. BKT12695
Clostridium botulinum C/D str. It1
Clostridium botulinum C/D str. Sp77
Clostridium botulinum CDC_1436
Clostridium botulinum CDC_297
Clostridium botulinum CFSAN001627
Clostridium botulinum CFSAN001628
Clostridium botulinum CFSAN002367
Clostridium botulinum CFSAN002369
Clostridium botulinum D str. 16868
Clostridium botulinum F 357
Clostridium botulinum F str. 230613
Clostridium botulinum F str. Langeland
Clostridium botulinum H04402 065
Clostridium botulinum NCTC 2916
Clostridium botulinum Prevot_594
Clostridium botulinum V891
Clostridium botulinum
Clostridium butyricum 5521
Clostridium butyricum 60E.3
Clostridium butyricum DKU-01
Clostridium butyricum DORA_1
Clostridium butyricum E4 str. BoNT E BL5262
Clostridium butyricum
Clostridium cadaveris
Clostridium carboxidivorans P7
Clostridium carboxidivorans
Clostridium carnis
Clostridium cellulovorans 743B
Clostridium cellulovorans
Clostridium chauvoei JF4335
Clostridium chromiireducens
Clostridium cochlearium
Clostridium colicanis DSM 13634
Clostridium combesii
Clostridium coskatii
Clostridium cuniculi
Clostridium cylindrosporum DSM 605
Clostridium cylindrosporum
Clostridium diolis
Clostridium disporicum
Clostridium drakei
Clostridium estertheticum subsp. estertheticum
Clostridium estertheticum
Clostridium faecium
Clostridium felsineum
Clostridium fermenticellae
Clostridium formicaceticum
Clostridium gallinarum
Clostridium gasigenes
Clostridium gelidum
Clostridium grantii DSM 8605
Clostridium grantii
Clostridium haemolyticum NCTC 8350
Clostridium haemolyticum NCTC 9693
Clostridium haemolyticum
Clostridium homopropionicum DSM 5847
Clostridium homopropionicum
Clostridium hydrogeniformans
Clostridium hydrogenum
Clostridium ihumii
Clostridium indicum
Clostridium intestinale DSM 6191
Clostridium intestinale URNW
Clostridium intestinale
Clostridium jeddahense
Clostridium kluyveri DSM 555
Clostridium kluyveri NBRC 12016
Clostridium kluyveri
Clostridium lacusfryxellense
Clostridium leptum CAG: 27
Clostridium ljungdahlii DSM 13528
Clostridium ljungdahlii
Clostridium lundense
Clostridium luticellarii
Clostridium magnum DSM 2767
Clostridium magnum
Clostridium manihotivorum
Clostridium massiliamazoniense
Clostridium mediterraneense
Clostridium merdae
Clostridium minihomine
Clostridium muellerianum
Clostridium neonatale
Clostridium niameyense
Clostridium novyi A str. 4540
Clostridium novyi A str. 4552
Clostridium novyi A str. 4570
Clostridium novyi A str. BKT29909
Clostridium novyi A str. GD211209
Clostridium novyi A str. NCTC 538
Clostridium novyi B str. ATCC 27606
Clostridium novyi B str. NCTC 9691
Clostridium novyi NT
Clostridium novyi
Clostridium oryzae
Clostridium paraputrificum
Clostridium pasteurianum BC1
Clostridium pasteurianum DSM 525 = ATCC 6013
Clostridium pasteurianum
Clostridium polyendosporum
Clostridium polynesiense
Clostridium porci
Clostridium puniceum
Clostridium ragsdalei P11
Clostridium ragsdalei
Clostridium rectalis
Clostridium saccharobutylicum
Clostridium saccharoperbutylacetonicum N1-4(HMT)
Clostridium saccharoperbutylacetonicum
Clostridium sartagoforme AAU1
Clostridium sartagoforme
Clostridium scatologenes
Clostridium senegalense
Clostridium septicum
Clostridium simiarum
Clostridium sp. 001
Clostridium sp. 1001270J_160509_D11
Clostridium sp. 2-1
Clostridium sp. 3-3
Clostridium sp. 42_12
Clostridium sp. 7_2_43FAA
Clostridium sp. AF27-2AA
Clostridium sp. AF32-12BH
Clostridium sp. AF46-12NS
Clostridium sp. AF46-9NS
Clostridium sp. AM33-3
Clostridium sp. AM34-11AC
Clostridium sp. AM42-4
Clostridium sp. AM45-5
Clostridium sp. AM49-4BH
Clostridium sp. AM51-4
Clostridium sp. ASBs410
Clostridium sp. ATCC BAA-442
Clostridium sp. AWRP
Clostridium sp. BL-8
Clostridium sp. BL8
Clostridium sp. BNL1100
Clostridium sp. C2-6-12
Clostridium sp. C8
Clostridium sp. CAG: 1013
Clostridium sp. CAG: 167
Clostridium sp. CAG: 242
Clostridium sp. CAG: 253
Clostridium sp. CAG: 277
Clostridium sp. CAG: 411
Clostridium sp. CAG: 505
Clostridium sp. CAG: 510
Clostridium sp. CAG: 590
Clostridium sp. CAG: 62
Clostridium sp. CAG: 632
Clostridium sp. CAG: 75
Clostridium sp. CAG: 91
Clostridium sp. CT7
Clostridium sp. D5
Clostridium sp. DJ247
Clostridium sp. DL-VIII
Clostridium sp. DMHC 10
Clostridium sp. DSM 8431
Clostridium sp. E02
Clostridium sp. FS41
Clostridium sp. HBUAS56010
Clostridium sp. HMP 27
Clostridium sp. HMSC19A10
Clostridium sp. HV4-5-A1G
Clostridium sp. Hs50
Clostridium sp. IBUN125C
Clostridium sp. JN-1
Clostridium sp. K25
Clostridium sp. KNHs205
Clostridium sp. Kas104-4
Clostridium sp. Kas106-4
Clostridium sp. Kas107-1
Clostridium sp. L74
Clostridium sp. LQ25
Clostridium sp. LS
Clostridium sp. MF28
Clostridium sp. MK11
Clostridium sp. MK12
Clostridium sp. MK31
Clostridium sp. MK8
Clostridium sp. MSTE9
Clostridium sp. Maddingley MBC34-26
Clostridium sp. Marseille-P2415
Clostridium sp. Marseille-P299
Clostridium sp. NCR
Clostridium sp. NJ4
Clostridium sp. OF09-36
Clostridium sp. OM02-18AC
Clostridium sp. OM05-6BH
Clostridium sp. OM05-9BH
Clostridium sp. SY8519
Clostridium sp. USBA 49
Clostridium sp. W14A
Clostridium sp. WLY-B-L2
Clostridium sp. YIM B02500
Clostridium sp. YIM B02506
Clostridium sp. YIM B02555
Clostridium sp. ZS1
Clostridium sp. cel8
Clostridium sporogenes ATCC 15579
Clostridium sporogenes PA 3679
Clostridium sporogenes
Clostridium sulfidigenes
Clostridium tepidiprofundi DSM 19306
Clostridium tetani 12124569
Clostridium tetani
Clostridium tetanomorphum DSM 665
Clostridium tetanomorphum
Clostridium thailandense
Clostridium tunisiense
Clostridium tyrobutyricum DIVETGP
Clostridium tyrobutyricum
Clostridium vincentii
Clostridium vitabionis
Clostridium zeae
Clostridium
Cnuella takakiae
Cobetia crustatorum
Cobetia sp. MC34
Codakia costata gill symbiont
Codakia orbicularis gill symbiont
Coffea canephora
Cognatazoarcus halotolerans
Cognaticolwellia aestuarii
Cognatishimia activa
Cognatiyoonia koreensis
Cohaesibacter haloalkalitolerans
Cohaesibacter intestini
Cohaesibacter marisflavi
Cohaesibacter sp. CAU 1516
Cohaesibacter sp. ES.047
Cohnella kolymensis
Cohnella laeviribosi
Cohnella lubricantis
Cohnella panacarvi
Cohnella sp. GbtcB17
Cohnella sp. JJ-181
Cohnella sp. OV330
Cohnella thermotolerans
Cohnella zeiphila
Coleofasciculus chthonoplastes CCY0002
Coleofasciculus chthonoplastes CCY9602
Coleofasciculus chthonoplastes CCY9603
Coleofasciculus chthonoplastes CCY9604
Coleofasciculus chthonoplastes CCY9605
Coleofasciculus chthonoplastes CCY9606
Coleofasciculus chthonoplastes CCY9607
Coleofasciculus chthonoplastes CCY9608
Coleofasciculus chthonoplastes PCC 7420
Coleofasciculus chthonoplastes
Coleofasciculus sp. FACHB-1120
Coleofasciculus sp. FACHB-125
Coleofasciculus sp. FACHB-129
Coleofasciculus sp. FACHB-501
Coleofasciculus sp. FACHB-542
Coleofasciculus sp. FACHB-64
Coleofasciculus sp. FACHB-712
Coleofasciculus sp. FACHB-SPT36
Coleofasciculus sp. FACHB-SPT9
Coleofasciculus sp. FACHB-T130
Coleofasciculus sp. LEGE 07081
Coleofasciculus sp. LEGE 07092
Collimonas sp. OK412
Collimonas sp. OK607
Collinsella aerofaciens
Collybiopsis luxurians FD-317 M1
Colwellia piezophila
Colwellia psychrerythraea
Colwellia sp. BRX10-3
Colwellia sp. MSW7
Colwellia sp. Phe_37
Colwellia sp. TT2012
Coriobacteriaceae bacterium CG2_30_59_20
Comamonadaceae bacterium CG_4_10_14_3_um_filter_60_42
Comamonadaceae bacterium CG_4_9_14_3_um_filter_60_33
Comamonadaceae bacterium PBBC1
Comamonadaceae bacterium PBBC2
Comamonadaceae bacterium
Comamonas aquatica
Comamonas granuli
Comamonas koreensis
Comamonas sp. B-9
Comamonas sp. C11
Comamonas sp. E6
Comamonas sp. JNW
Comamonas terrigena
Comamonas testosteroni ATCC 11996
Comamonas testosteroni
Comamonas thiooxydans
Compactonostoc shennongjiaensis CHAB5781
Conexibacter sp. DBS9H8
Conexibacter sp. SYSU D00693
Conexibacter sp. W3-3-2
Conexibacter woesei
Congregibacter litoralis KT71
Congregibacter litoralis
Consotaella salsifontis
Conyzicola lurida
Conyzicola nivalis
Copidosoma floridanum
Coprococcus catus GD/7
Coprococcus catus
Coprococcus comes ATCC 27758
Coprococcus comes CAG: 19
Coprococcus comes
Coprococcus eutactus
Coprococcus sp. 43 8
Coprococcus sp. AF102-57
Coprococcus sp. B2-R-112
Coprococcus
Coraliihabitans acroporae
Coraliomargarita akajimensis DSM 45221
Coraliomargarita akajimensis
Coraliomargarita sp. WMMB3
Corallincola holothuriorum
Corallincola luteus
Corallincola spongiicola
Corallincola
Corallococcus aberystwythensis
Corallococcus carmarthensis
Corallococcus coralloides DSM 2259
Corallococcus coralloides
Corallococcus exercitus
Corallococcus exiguus
Corallococcus interemptor
Corallococcus llansteffanensis
Corallococcus macrosporus DSM 14697
Corallococcus macrosporus
Corallococcus praedator
Corallococcus sicarius
Corallococcus soli
Corallococcus sp. AB004
Corallococcus sp. AB011P
Corallococcus sp. AB030
Corallococcus sp. AB032C
Corallococcus sp. AB038B
Corallococcus sp. AB045
Corallococcus sp. AB049A
Corallococcus sp. AB050B
Corallococcus sp. BB11-1
Corallococcus sp. CA031C
Corallococcus sp. CA041A
Corallococcus sp. CA047B
Corallococcus sp. CA049B
Corallococcus sp. CA053C
Corallococcus sp. CA054B
Corallococcus sp. EGB
Corallococcus sp. NCSPR001
Corallococcus sp. bb12-1
Corallococcus terminator
Corallococcus
Comamonadaceae bacterium CHKCI002
Coriobacteriia bacterium
Corticibacterium populi
Corynebacterium casei
Corynebacterium diphtheriae 241
Corynebacterium diphtheriae 31A
Corynebacterium diphtheriae BH8
Corynebacterium diphtheriae C7 (beta)
Corynebacterium diphtheriae CDCE 8392
Corynebacterium diphtheriae HC01
Corynebacterium diphtheriae HC02
Corynebacterium diphtheriae HC03
Corynebacterium diphtheriae HC04
Corynebacterium diphtheriae INCA 402
Corynebacterium diphtheriae PW8
Corynebacterium diphtheriae VA01
Corynebacterium diphtheriae bv. mitis
Corynebacterium diphtheriae
Corynebacterium epidermidicanis
Corynebacterium kroppenstedtii
Corynebacterium marinum DSM 44953
Corynebacterium neomassiliense
Corynebacterium provencense
Corynebacterium pseudodiphtheriticum
Corynebacterium sp. 35RC1
Corynebacterium sphenisci DSM 44792
Corynebacterium variabile
Couchioplanes caeruleus
Coxiella sp. DG 40
Coxiella-like endosymbiont of Rhipicephalus sanguineus
Crassaminicella profunda
Crateriforma conspicua
Crateriforma spongiae
Crateriforma
Crenobacter cavernae
Crinalium epipsammum PCC 9333
Crinalium epipsammum
Croceibacterium mercuriale
Croceicoccus naphthovorans
Croceicoccus sp. YJ47
Croceivirga radicis
Crocosphaera chwakensis CCY0110
Crocosphaera chwakensis
Crocosphaera sp. 1 str. KO20B5
Crocosphaera sp. 1 str. KO30D1
Crocosphaera sp. 1 str. KO38CU6
Crocosphaera subtropica ATCC 51142
Crocosphaera subtropica
Crocosphaera watsonii WH 0003
Crocosphaera watsonii WH 0005
Crocosphaera watsonii WH 0401
Crocosphaera watsonii WH 0402
Crocosphaera watsonii WH 8501
Crocosphaera watsonii WH 8502
Crocosphaera watsonii
Cronbergia siamensis NQAIF308
Cronbergia siamensis TISTR 8012
Cronobacter dublinensis
Cronobacter malonaticus
Cronobacter sakazakii
Cronobacter turicensis
Cronobacter universalis
Cryobacterium arcticum
Cryobacterium aureum
Cryobacterium flavum
Cryobacterium levicorallinum
Cryobacterium luteum
Cryobacterium melibiosiphilum
Cryobacterium psychrotolerans
Cryobacterium roopkundense
Cryobacterium ruanii
Cryobacterium soli
Cryobacterium sp. HLT2-23
Cryobacterium sp. Hb1
Cryobacterium sp. Hh11
Cryobacterium sp. Hh14
Cryobacterium sp. Hh38
Cryobacterium sp. Hh4
Cryobacterium sp. Hh7
Cryobacterium sp. Hz16
Cryobacterium sp. Hz9
Cryobacterium sp. M15
Cryobacterium sp. M23
Cryobacterium sp. M25
Cryobacterium sp. M91
Cryobacterium sp. M96
Cryobacterium sp. MDT1-3
Cryobacterium sp. N19
Cryobacterium sp. N21
Cryobacterium sp. N22
Cryobacterium sp. PAMC25264
Cryobacterium sp. RHLS22-1
Cryobacterium sp. RHLT2-21
Cryobacterium sp. SO1
Cryobacterium sp. Sr39
Cryobacterium sp. Sr3
Cryobacterium sp. Sr47
Cryobacterium sp. Sr54
Cryobacterium sp. Sr59
Cryobacterium sp. Sr8
Cryobacterium sp. TMB1-7
Cryobacterium sp. TMS1-13-1
Cryobacterium sp. TMS1-20-1
Cryobacterium sp. TMT1-1
Cryobacterium sp. TMT1-2-1
Cryobacterium sp. TMT1-2-2
Cryobacterium sp. TMT1-23-1
Cryobacterium sp. TMT1-3
Cryobacterium sp. TMT1-51
Cryobacterium sp. TMT1-62
Cryobacterium sp. TMT1-66-1
Cryobacterium sp. TMT2-15-1
Cryobacterium sp. TMT2-17-1
Cryobacterium sp. TMT2-42-4
Cryobacterium sp. TMT3-29-2
Cryobacterium sp. TmT2-48-2
Cryobacterium sp. Y11
Cryobacterium sp. Y29
Cryobacterium sp. Y50
Cryobacterium sp. Y57
Cryobacterium sp. Y62
Cryobacterium sp. Y82
Cryobacterium tepidiphilum
Cryobacterium zongtaii
Cryobacterium
Cryptosporangium arvum DSM 44712
Cryptosporangium arvum
Cryptosporangium aurantiacum
Cryptosporangium phraense
Cryptotermes secundus
Cuniculiplasma divulgatum
Cupide sulfovibrio sp. HK-II
Cupide sulfovibrio sp. SRB-5
Cupidesulfovibrio termitidis
Cupide sulfovibrio
Cupriavidus alkaliphilus
Cupriavidus basilensis
Cupriavidus metallidurans
Cupriavidus necator H16
Cupriavidus necator
Cupriavidus neocaledonicus
Cupriavidus oxalaticus
Cupriavidus pampae
Cupriavidus pauculus
Cupriavidus pinatubonensis
Cupriavidus plantarum
Cupriavidus sp. 2SB
Cupriavidus sp. AU9028
Cupriavidus sp. D384
Cupriavidus sp. EM10
Cupriavidus sp. JNVU MP15
Cupriavidus sp. JNVU MP3
Cupriavidus sp. JNVU MP6
Cupriavidus sp. LEh25
Cupriavidus sp. SK-3
Cupriavidus sp. SW-Y-13
Cupriavidus sp. SWF65028
Cupriavidus sp. SWF65033
Cupriavidus sp. SWF66133
Cupriavidus sp. SWF66166
Cupriavidus sp. SWF66194
Cupriavidus sp. SWF66294
Cupriavidus sp. SWF66316
Cupriavidus sp. SWF66322
Cupriavidus sp. SWF67044
Cupriavidus sp. U2
Cupriavidus sp. UGS-1
Cupriavidus sp. UYMAM01A
Cupriavidus sp. UYMAM01B
Cupriavidus sp. UYMM02A
Cupriavidus sp. UYMM14A
Cupriavidus sp. UYMMa02A
Cupriavidus sp. UYMRA05A
Cupriavidus sp. UYMRA10A
Cupriavidus sp. UYMRE01A
Cupriavidus sp. UYMRE04A
Cupriavidus sp. UYMS01A
Cupriavidus sp. UYMS13B
Cupriavidus sp. UYPR2.512
Cupriavidus sp. UYPR2.54
Cupriavidus sp. UYPR2.55
Cupriavidus sp. WGtm5
Cupriavidus sp. amp18
Cupriavidus sp. amp34
Cupriavidus sp. amp45
Cupriavidus sp. amp51
Cupriavidus sp. amp6
Cupriavidus sp. cmp17
Cupriavidus sp. cmp29
Cupriavidus sp. cmp2
Cupriavidus sp. cmp36
Cupriavidus sp. cmp47
Cupriavidus sp. cmp52
Cupriavidus sp. cmp54
Cupriavidus sp. cmp57
Cupriavidus sp. cmp60
Cupriavidus sp. cmp63
Cupriavidus sp. cmp70
Cupriavidus sp. ip2.100
Cupriavidus sp. ip2.14
Cupriavidus sp. ip2.30
Cupriavidus sp. ip2.35
Cupriavidus sp. ip2.47
Cupriavidus sp. ip2.61
Cupriavidus sp. ip2.89
Cupriavidus sp. mapud1.3
Cupriavidus sp. mapud10.1
Cupriavidus sp. mapud10.5
Cupriavidus sp. mapud3.1
Cupriavidus sp. mapud3.2
Cupriavidus sp. mapud3.4
Cupriavidus sp. mapud4.3
Cupriavidus sp. mapud4.4
Cupriavidus sp. mapud4.5
Cupriavidus sp. mapud5.1
Cupriavidus sp. mapud5.4
Cupriavidus sp. mapud7.4
Cupriavidus sp. mapud7.6
Cupriavidus sp. mapud8.1
Cupriavidus sp. mapud8.2
Cupriavidus sp. mapud8.4
Cupriavidus sp. mcg1
Cupriavidus sp. mcg5
Cupriavidus sp. mip11
Cupriavidus sp. mip15
Cupriavidus sp. mip2
Cupriavidus sp. mip34
Cupriavidus sp. mpj6
Cupriavidus sp. mpl20
Cupriavidus sp. mpl29
Cupriavidus sp. mpmg1
Cupriavidus sp. mpmg2
Cupriavidus sp. mpmg4
Cupriavidus sp. mpmg5
Cupriavidus sp. mpp1.13
Cupriavidus sp. mpp1.16
Cupriavidus sp. mpp1.1
Cupriavidus sp. mpp1.2
Cupriavidus sp. mpp1.3
Cupriavidus sp. mpp1.6
Cupriavidus sp. mpp2.10
Cupriavidus sp. mpp2.18
Cupriavidus sp. mpp2.1
Cupriavidus sp. mpp2.22
Cupriavidus sp. mpp2.26
Cupriavidus sp. mpp2.7
Cupriavidus sp. mppg11
Cupriavidus sp. mppg18
Cupriavidus sp. mppg22
Cupriavidus sp. mppg25
Cupriavidus sp. mppg2
Cupriavidus sp. mppg30
Cupriavidus sp. mppg35
Cupriavidus sp. mppg36
Cupriavidus sp. mppg6
Cupriavidus sp. mppg8
Cupriavidus sp. mym10
Cupriavidus sp. mym1
Cupriavidus sp. myp8
Cupriavidus sp. pp2.22
Cupriavidus sp. pp2.3
Cupriavidus sp. pp2.41
Cupriavidus sp. pp2.43
Cupriavidus sp. pp2.49
Cupriavidus sp. pp2.54
Cupriavidus sp. pp2.75
Cupriavidus sp. pp2.90
Cupriavidus sp. pp2.92
Cupriavidus sp. tpig.1a
Cupriavidus sp. tpig.6a
Cupriavidus sp. tpud1.6
Cupriavidus sp. tpud1.7
Cupriavidus sp. tpud17.3
Cupriavidus sp. tpud19.2
Cupriavidus sp. tpud19.4
Cupriavidus sp. tpud2.4
Cupriavidus sp. tpud20.2
Cupriavidus sp. tpud23.3
Cupriavidus sp. tpud27.2
Cupriavidus sp. tpud27.6
Cupriavidus sp. tpud28.1
Cupriavidus sp. tpud31.3
Cupriavidus sp. tpud31.5
Cupriavidus sp. tpud32.2
Cupriavidus sp.
Cupriavidus taiwanensis LMG 19424
Cupriavidus taiwanensis
Cupriavidus
Curtobacterium albidum
Curtobacterium allii
Curtobacterium citreum
Curtobacterium flaccumfaciens
Curtobacterium herbarum
Curtobacterium luteum
Curtobacterium oceanosedimentum
Curtobacterium pusillum
Curtobacterium sp. ‘Ferrero’
Curtobacterium sp. 18060
Curtobacterium sp. 24E2
Curtobacterium sp. 314Chir4.1
Curtobacterium sp. AG1037
Curtobacterium sp. B18
Curtobacterium sp. BH-2-1-1
Curtobacterium sp. C1
Curtobacterium sp. C2H10
Curtobacterium sp. Csp2
Curtobacterium sp. GD1
Curtobacterium sp. ISL-83
Curtobacterium sp. KBS0715
Curtobacterium sp. L6-1
Curtobacterium sp. Leaf154
Curtobacterium sp. Leaf183
Curtobacterium sp. Leaf261
Curtobacterium sp. MCBA15_004
Curtobacterium sp. MCBA15_005
Curtobacterium sp. MCBA15_008
Curtobacterium sp. MCBA15_012
Curtobacterium sp. MCBA15_013
Curtobacterium sp. MCBA15_016
Curtobacterium sp. MCBD17_026
Curtobacterium sp. MCBD17_030
Curtobacterium sp. MCLR17_007
Curtobacterium sp. MCLR17_031
Curtobacterium sp. MCLR17_032
Curtobacterium sp. MCLR17_039
Curtobacterium sp. MCLR17_040
Curtobacterium sp. MCLR17_042
Curtobacterium sp. MCLR17_044
Curtobacterium sp. MCLR17_045
Curtobacterium sp. MCLR17_054
Curtobacterium sp. MCLR17_055
Curtobacterium sp. MCLR17_058
Curtobacterium sp. MCPF17_002
Curtobacterium sp. MCPF17_003
Curtobacterium sp. MCPF17_011
Curtobacterium sp. MCPF17_018
Curtobacterium sp. MCPF17_021
Curtobacterium sp. MCPF17_031
Curtobacterium sp. MCPF17_046
Curtobacterium sp. MCPF17_051
Curtobacterium sp. MCPF17_052
Curtobacterium sp. MCSS17_005
Curtobacterium sp. MCSS17_006
Curtobacterium sp. MCSS17_011
Curtobacterium sp. MCSS17_016
Curtobacterium sp. ME12
Curtobacterium sp. MMLR14_010
Curtobacterium sp. ODYSSEY 48 V2
Curtobacterium sp. P97
Curtobacterium sp. PhB115
Curtobacterium sp. PhB130
Curtobacterium sp. PhB136
Curtobacterium sp. R-46312
Curtobacterium sp. SL109
Curtobacterium sp. UCD-KPL2560
Curtobacterium sp. UNCCL17
Curtobacterium sp. UNCCL20
Curtobacterium sp. VKM Ac-1376
Curtobacterium sp. VKM Ac-2884
Curtobacterium sp. VKM Ac-2922
Curtobacterium sp. WW7
Curtobacterium sp. YC1
Curtobacterium sp. YR515
Curtobacterium sp. ZW137
Curtobacterium
Curvibacter gracilis
Curvibacter lanceolatus
Curvibacter putative symbiont of Hydra magnipapillata
Curvibacter sp. PAE-UM
Curvibacter sp. RS43
Curvibacter sp.
Curvibacter
Curvivirga aplysinae
Cuscuta australis
Cuspidothrix issatschenkoi CHARLIE-1
Cuspidothrix issatschenkoi UADFA1
Cuspidothrix issatschenkoi
Cutibacterium granulosum
Cyanistes caeruleus
Cyanobacterium aponinum PCC 10605
Cyanobacterium aponinum
Cyanobacterium sp. CLg1
Cyanobacterium sp. NBRC 102756
Cyanobacterium sp. uoEpiScrs1
Cyanobacterium stanieri PCC 7202
Cyanobium gracile PCC 6307
Cyanobium gracile
Cyanobium sp. Candia 9D4
Cyanobium sp. Copco_Reservoir_LC18
Cyanobium sp. To12R1
Cyanobium sp.
Cyanobium usitatum
Cyanophyceae
Cyanosarcina cf. burmensis CCALA 770
Cyanothece sp. BG0011
Cyanothece sp. LCRNK_21
Cyanothece sp. TW3
Cyanothece sp. WH 8902
Cyclobacterium qasimii
Cycloclasticus pugetii
Cycloclasticus sp. Phe_18
Cycloclasticus zancles 78-ME
Cycloclasticus
Cyclonatronum proteinivorum
Cylindrospermopsis africana PMC115.02
Cylindrospermopsis curvispora GIHE-G1
Cylindrospermopsis curvispora PMC144.02
Cylindrospermopsis curvispora PMC262.06
Cylindrospermopsis curvispora
Cylindrospermopsis raciborskii C03
Cylindrospermopsis raciborskii C04
Cylindrospermopsis raciborskii C07
Cylindrospermopsis raciborskii CHAB141
Cylindrospermopsis raciborskii CHAB353
Cylindrospermopsis raciborskii CHAB358
Cylindrospermopsis raciborskii CS-505
Cylindrospermopsis raciborskii Cr2010
Cylindrospermopsis raciborskii FLcultureE-3
Cylindrospermopsis raciborskii FLcultureL-5
Cylindrospermopsis raciborskii FSS-127
Cylindrospermopsis raciborskii GIHE 2018
Cylindrospermopsis raciborskii KLL07
Cylindrospermopsis raciborskii LD-D
Cylindrospermopsis raciborskii LD-E
Cylindrospermopsis raciborskii LD-F
Cylindrospermopsis raciborskii LD-G
Cylindrospermopsis raciborskii LD-I
Cylindrospermopsis raciborskii LG-L
Cylindrospermopsis raciborskii MVCC14
Cylindrospermopsis raciborskii MVCC19
Cylindrospermopsis raciborskii NJ3
Cylindrospermopsis raciborskii PMC114.02
Cylindrospermopsis raciborskii PMC117.02
Cylindrospermopsis raciborskii PMC118.02
Cylindrospermopsis raciborskii PMC145.02
Cylindrospermopsis raciborskii PMC98.14
Cylindrospermopsis raciborskii PMC99.06
Cylindrospermopsis raciborskii PMC99.08
Cylindrospermopsis raciborskii PMC99.12
Cylindrospermopsis raciborskii R104
Cylindrospermopsis raciborskii S01
Cylindrospermopsis raciborskii S05
Cylindrospermopsis raciborskii S06
Cylindrospermopsis raciborskii S07
Cylindrospermopsis raciborskii S10
Cylindrospermopsis raciborskii S14
Cylindrospermopsis raciborskii TAU-MAC 1414
Cylindrospermopsis raciborskii UAM520
Cylindrospermopsis raciborskii UAM544
Cylindrospermopsis raciborskii str. Goon
Cylindrospermopsis raciborskii
Cylindrospermopsis sp. CR12
Cylindrospermopsis
Cylindrospermum muscicola ACSSI 140
Cylindrospermum muscicola CG16
Cylindrospermum muscicola Ind12
Cylindrospermum muscicola Ind13
Cylindrospermum skujae ACSSI 112
Cylindrospermum sp. ACSSI 040
Cylindrospermum sp. ACSSI 041
Cylindrospermum sp. ACSSI 042
Cylindrospermum sp. CG17
Cylindrospermum sp. FACHB-282
Cylindrospermum sp. Ind14
Cylindrospermum sp. MN-4B
Cylindrospermum sp. NDUPC005
Cylindrospermum sp. NIES-4074
Cylindrospermum sp. SU01
Cylindrospermum sp.
Cylindrospermum stagnale CG18
Cylindrospermum stagnale Ind15
Cylindrospermum stagnale PCC 7417
Cylindrospermum stagnale
Cypionkella psychrotolerans
Cysteiniphilum halobium
Cystobacter fuscus
Cystobacter gracilis
Cytobacillus firmus DS1
Cytobacillus firmus
Cytobacillus kochii
Cytobacillus oceanisediminis 2691
Cytobacillus oceanisediminis
Cytobacillus praedii
Cytobacillus solani
Cytobacillus
Cytophaga aurantiaca
Cytophaga hutchinsonii
Dactylococcopsis salina PCC 8305
Dactylosporangium aurantiacum
Dactylosporangium fulvum
Dactylosporangium matsuzakiense
Dactylosporangium roseum
Dactylosporangium siamense
Dactylosporangium sp. AC04546
Dactylosporangium sucinum
Daejeonella lutea
Daejeonella oryzae
Daejeonella rubra
Dankookia rubra
Daphnia pulex
Dapis pleousa FFP11-4
Dapis pleousa FFP12-7
Dapis pleousa FSI10-1
Dapis pnigousa NAB11-15
Dapis pnigousa NAB11-16
Dasania marina
Dawidia cretensis
Dawidia soli
Dechloromonas denitrificans
Dechloromonas hortensis
Dechloromonas sp. A34
Dechloromonas sp. H13
Dechloromonas sp. HYN0024
Dechloromonas sp. SIUL
Dechloromonas sp. TW-R-39-2
Dechloromonas sp.
Deefgea rivuli
Deefgea sp. CFH1-16
Deefgea tanakiae
Deefgea
Deferribacter desulfuricans SSM1
Deferrisoma camini
Defluviicoccus sp.
Defluviicoccus vanus
Defluviitalea phaphyphila
Defluviitalea raffinosedens
Dehalobacter restrictus DSM 9455
Dehalobacter restrictus
Dehalobacter sp. 12DCB1
Dehalobacter sp. 14DCB1
Dehalobacter sp. CF
Dehalobacter sp. DCA
Dehalobacter sp. DCM
Dehalobacter sp. MCB1
Dehalobacter sp. UNSWDHB
Dehalobacterium formicoaceticum
Dehalococcoidales bacterium
Dehalococcoides mccartyi 195
Dehalococcoides mccartyi CG1
Dehalococcoides mccartyi CG4
Dehalococcoides mccartyi CG5
Dehalococcoides mccartyi IBARAKI
Dehalococcoides mccartyi
Dehalococcoides
Dehalococcoidia bacterium CG2_30_46_9
Dehalococcoidia bacterium DG_18
Dehalococcoidia bacterium SM23_28_2
Dehalococcoidia bacterium
Dehalogenimonas alkenigignens
Dehalogenimonas lykanthroporepellens BL-DC-9
Dehalogenimonas sp. WBC-2
Deinococcus aerophilus
Deinococcus alpinitundrae
Deinococcus aquaedulcis
Deinococcus aquaticus
Deinococcus arboris
Deinococcus arcticus
Deinococcus betulae
Deinococcus cavernae
Deinococcus cellulosilyticus
Deinococcus daejeonensis
Deinococcus ficus
Deinococcus fonticola
Deinococcus geothermalis
Deinococcus grandis
Deinococcus humi
Deinococcus indicus
Deinococcus knuensis
Deinococcus koreensis
Deinococcus malanensis
Deinococcus marmoris
Deinococcus metalli
Deinococcus misasensis
Deinococcus multiflagellatus
Deinococcus peraridilitoris
Deinococcus radiodurans
Deinococcus radiopugnans
Deinococcus roseus
Deinococcus ruber
Deinococcus seoulensis
Deinococcus sp. 12RED42
Deinococcus sp. 14RED07
Deinococcus sp. 6GRE01
Deinococcus sp. 6YEL10
Deinococcus sp. AJ005
Deinococcus sp. HSC-46F16
Deinococcus sp. LM3
Deinococcus sp. QL22
Deinococcus sp. RIT780
Deinococcus sp. RM
Deinococcus sp. TS-293
Deinococcus wulumugiensis
Deinococcus yavapaiensis
Delftia acidovorans SPH-1
Delftia acidovorans
Delftia lacustris
Delftia sp. BR1
Delftia sp. GW456-R20
Delftia sp. JD2
Delftia sp. ZNC0008
Delftia tsuruhatensis
Deltaproteobacteria bacterium CG11_big_fil_rev_8_21_14_0_20_45_16
Deltaproteobacteria bacterium CG11_big_fil_rev_8_21_14_0_20_49_13
Deltaproteobacteria bacterium CG12_big_fil_rev_8_21_14_0_65_43_10
Deltaproteobacteria bacterium CG23_combo_of_CG06-09_8_20_14_all_60_8
Deltaproteobacteria bacterium CG2_30_63_29
Deltaproteobacteria bacterium CG_4_10_14_0_8_um_filter_43_1_2
Deltaproteobacteria bacterium CG_4_10_14_3_um_filter_60_8
Deltaproteobacteria bacterium CG_4_9_14_3_um_filter_44_9
Deltaproteobacteria bacterium CG_4_9_14_3_um_filter_51_14
Deltaproteobacteria bacterium CG_4_9_14_3_um_filter_63_12
Deltaproteobacteria bacterium DG_8
Deltaproteobacteria bacterium HGW-Deltaproteobacteria-10
Deltaproteobacteria bacterium HGW-Deltaproteobacteria-13
Deltaproteobacteria bacterium HGW-Deltaproteobacteria-14
Deltaproteobacteria bacterium HGW-Deltaproteobacteria-16
Deltaproteobacteria bacterium HGW-Deltaproteobacteria-18
Deltaproteobacteria bacterium HGW-Deltaproteobacteria-20
Deltaproteobacteria bacterium HGW-Deltaproteobacteria-24
Deltaproteobacteria bacterium HGW-Deltaproteobacteria-3
Deltaproteobacteria bacterium HGW-Deltaproteobacteria-4
Deltaproteobacteria bacterium HGW-Deltaproteobacteria-6
Deltaproteobacteria bacterium HGW-Deltaproteobacteria-8
Deltaproteobacteria bacterium SG8_13
Deltaproteobacteria bacterium SM23_61
Deltaproteobacteria bacterium
Demetria terragena
Deminuibacter soli
Dendrobium catenatum
Dendronalium phyllosphericum CENA358
Dendronalium phyllosphericum CENA369
Dendronalium phyllosphericum CENA389
Dendronalium phyllosphericum
Dendrosporobacter quercicolus
Denitrificimonas caeni
Denitrobaculum tricleocarpae
Denitromonas sp. IR12
Denitrovibrio acetiphilus DSM 12809
Denitrovibrio acetiphilus
Denitrovibrio sp.
Dermacoccus abyssi
Dermacoccus barathri
Dermacoccus nishinomiyaensis
Dermacoccus sp. 147Ba
Dermacoccus sp. PAMC28757
Dermacoccus sp. PE3
Dermacoccus sp. Tok2021
Dermacoccus
Derxia gummosa
Derxia lacustris
Derxia sp. P8-3
Desertifilum sp. FACHB-866
Desertifilum sp. IPPAS B-1220
Desmonostoc muscorum Ind33
Desmonostoc muscorum Ind34
Desmonostoc muscorum NDUPC006
Desmonostoc muscorum SN0603
Desmonostoc muscorum UTAD_N213
Desmonostoc muscorum UTEX LB 1933
Desmonostoc muscorum
Desmonostoc sp. CENA362
Desmonostoc sp. CENA363
Desmonostoc sp. CENA365
Desmonostoc sp. CENA371
Desmonostoc sp. CENA383
Desmonostoc sp. CENA386
Desmonostoc sp. MN-10A
Desmonostoc sp. MN-10B
Desmonostoc sp. MN-10D
Desmonostoc sp. MN-11A
Desmonostoc sp. MN-2C
Desnuesiella massiliensis
Desulfacinum hydrothermale DSM 13146
Desulfacinum hydrothermale
Desulfacinum infernum DSM 9756
Desulfacinum infernum
Desulfallas sp. Bu1-1
Desulfamplus magnetovallimortis
Desulfarculus baarsii DSM 2075
Desulfarculus baarsii
Desulfarculus sp.
Desulfatibacillum aliphaticivorans
Desulfatibacillum alkenivorans DSM 16219
Desulfatibacillum alkenivorans
Desulfatiglans anilini
Desulfatirhabdium butyrativorans
Desulfatitalea sp. BRH c12
Desulfatitalea sp. M08but
Desulfatitalea tepidiphila
Desulfitibacter alkalitolerans
Desulfitibacter sp. BRH_c19
Desulfitobacterium chlororespirans DSM 11544
Desulfitobacterium chlororespirans
Desulfitobacterium dehalogenans ATCC 51507
Desulfitobacterium dehalogenans
Desulfitobacterium dichloroeliminans LMG P-21439
Desulfitobacterium dichloroeliminans
Desulfitobacterium hafniense DCB-2
Desulfitobacterium hafniense DP7
Desulfitobacterium hafniense Y51
Desulfitobacterium hafniense
Desulfitobacterium metallireducens DSM 15288
Desulfitobacterium metallireducens
Desulfitobacterium sp. LBE
Desulfitobacterium sp. PCE1
Desulfitobacterium
Desulfobacca acetoxidans DSM 11109
Desulfobacca acetoxidans
Desulfobacca sp. 4484 104
Desulfobacter curvatus
Desulfobacter hydrogenophilus
Desulfobacter latus
Desulfobacter postgatei 2ac9
Desulfobacter postgatei
Desulfobacter vibrioformis
Desulfobacteraceae bacterium 4484_190.2
Desulfobacteraceae bacterium 4484_190.3
Desulfobacteraceae bacterium 4572_187
Desulfobacteraceae bacterium 4572_19
Desulfobacteraceae bacterium 4572_35.1
Desulfobacteraceae bacterium 4572_35.2
Desulfobacteraceae bacterium 4572_88
Desulfobacteraceae bacterium Eth-SRB1
Desulfobacteraceae bacterium Eth-SRB2
Desulfobacteraceae bacterium IS3
Desulfobacteraceae bacterium SEEP-SAG10
Desulfobacteraceae bacterium
Desulfobacterales bacterium CG23_combo_of_CG06-09_8_20_14_all_51_8
Desulfobacterales bacterium CG2_30_60_27
Desulfobacterales bacterium SG8_35_2
Desulfobacterales bacterium
Desulfobacula toluolica Tol2
Desulfobacula toluolica
Desulfobacula
Desulfobaculum xiamenense
Desulfobotulus alkaliphilus
Desulfobotulus mexicanus
Desulfobotulus sp. H1
Desulfobulbaceae bacterium A2
Desulfobulbaceae bacterium BRH_c16a
Desulfobulbaceae bacterium
Desulfobulbus alkaliphilus
Desulfobulbus elongatus
Desulfobulbus propionicus DSM 2032
Desulfobulbus propionicus
Desulfobulbus rhabdoformis
Desulfobulbus sp. Tol-SR
Desulfobulbus sp.
Desulfocapsa sulfexigens DSM 10523
Desulfocapsa sulfexigens
Desulfocarbo indianensis
Desulfocicer vacuolatum DSM 3385
Desulfocicer vacuolatum
Desulfocucumis palustris
Desulfocurvibacter africanus PCS
Desulfocurvibacter africanus subsp. africanus str. Walvis Bay
Desulfocurvibacter africanus
Desulfocurvus vexinensis
Desulfofalx alkaliphila
Desulfofarcimen acetoxidans DSM 771
Desulfofarcimen acetoxidans
Desulfoferrobacter suflitae
Desulfofundulus kuznetsovii DSM 6115
Desulfofundulus salinum
Desulfofundulus thermobenzoicus
Desulfofundulus thermocisternus
Desulfofundulus thermosubterraneus DSM 16057
Desulfofundulus
Desulfofustis glycolicus DSM 9705
Desulfofustis glycolicus
Desulfoglaeba alkanexedens ALDC
Desulfoglaeba alkanexedens
Desulfogranum japonicum
Desulfogranum marinum
Desulfogranum mediterraneum
Desulfohalobium retbaense DSM 5692
Desulfohalobium retbaense
Desulfohalovibrio alkalitolerans DSM 16529
Desulfohalovibrio alkalitolerans
Desulfohalovibrio reitneri
Desulfolithobacter dissulfuricans
Desulfolucianica intricata
Desulfoluna butyratoxydans
Desulfoluna sp. ASN36
Desulfoluna spongiiphila
Desulfolutivibrio sulfoxidireducens
Desulfomicrobium apsheronum
Desulfomicrobium baculatum DSM 4028
Desulfomicrobium baculatum
Desulfomicrobium escambiense
Desulfomicrobium macestii
Desulfomicrobium norvegicum
Desulfomicrobium orale DSM 12838
Desulfomicrobium sp. ZS1
Desulfomicrobium
Desulfomonile tiedjei DSM 6799
Desulfomonile tiedjei
Desulfonatronospira sp. MSAO_Bac3
Desulfonatronospira thiodismutans ASO3-1
Desulfonatronospira thiodismutans
Desulfonatronovibrio halophilus
Desulfonatronovibrio hydrogenovorans
Desulfonatronovibrio magnus
Desulfonatronum lacustre
Desulfonatronum sp. SC1
Desulfonatronum thioautotrophicum
Desulfonatronum thiodismutans
Desulfonatronum thiosulfatophilum
Desulfonauticus sp. 38_4375
Desulfonauticus submarinus
Desulfonema ishimotonii
Desulfonema limicola str. Jadebusen
Desulfonema limicola
Desulfonema magnum
Desulfopila aestuarii DSM 18488
Desulfopila aestuarii
Desulfopila sp. IMCC35006
Desulfopila sp. IMCC35008
Desulforadius tongensis
Desulforamulus aeronauticus DSM 10349
Desulforamulus aeronauticus
Desulforamulus ferrireducens
Desulforamulus hydrothermalis Lam5 = DSM 18033
Desulforamulus hydrothermalis
Desulforamulus profundi
Desulforamulus putei DSM 12395
Desulforamulus putei
Desulforamulus reducens MI-1
Desulforamulus reducens
Desulforamulus ruminis DSM 2154
Desulforamulus ruminis
Desulforapulum autotrophicum HRM2
Desulforapulum autotrophicum
Desulforegula conservatrix
Desulforhabdus amnigena
Desulforhabdus sp. TSK
Desulforhopalus singaporensis
Desulforhopalus sp. IMCC35007
Desulforhopalus vacuolatus
Desulfosarcina alkanivorans
Desulfosarcina cetonica
Desulfosarcina ovata subsp. ovata
Desulfosarcina ovata subsp. sediminis
Desulfosarcina ovata
Desulfosarcina sp. BuS5
Desulfosarcina widdelii
Desulfoscipio geothermicus DSM 3669
Desulfoscipio geothermicus
Desulfoscipio gibsoniae DSM 7213
Desulfoscipio gibsoniae
Desulfosediminicola flagellatus
Desulfosediminicola ganghwensis
Desulfospira joergensenii
Desulfosporosinus acididurans
Desulfosporosinus acidiphilus SJ4
Desulfosporosinus acidiphilus
Desulfosporosinus fructosivorans
Desulfosporosinus hippei DSM 8344
Desulfosporosinus hippei
Desulfosporosinus lacus DSM 15449
Desulfosporosinus meridiei DSM 13257
Desulfosporosinus meridiei
Desulfosporosinus metallidurans
Desulfosporosinus orientis DSM 765
Desulfosporosinus orientis
Desulfosporosinus sp. BG
Desulfosporosinus sp. BICA1-9
Desulfosporosinus sp. BRH_c37
Desulfosporosinus sp. FKA
Desulfosporosinus sp. FKB
Desulfosporosinus sp. HMP52
Desulfosporosinus sp. I2
Desulfosporosinus sp. OT
Desulfosporosinus sp. SRJS8
Desulfosporosinus sp. Sb-LF
Desulfosporosinus sp. Tol-M
Desulfosporosinus youngiae DSM 17734
Desulfosporosinus youngiae
Desulfosporosinus
Desulfosudis oleivorans Hxd3
Desulfosudis oleivorans
Desulfotalea psychrophila LSv54
Desulfotalea psychrophila
Desulfotalea sp.
Desulfotignum balticum
Desulfotignum phosphitoxidans DSM 13687
Desulfotignum phosphitoxidans
Desulfotomaculum arcticum
Desulfotomaculum copahuensis
Desulfotomaculum nigrificans CO-1-SRB
Desulfotomaculum nigrificans DSM 574
Desulfotomaculum nigrificans
Desulfotomaculum sp. 46_296
Desulfotomaculum sp. 46 80
Desulfotomaculum sp. BICA1-6
Desulfotruncus alcoholivorax
Desulfotruncus arcticus
Desulfovermiculus halophilus
Desulfovibrio aminophilus
Desulfovibrio cuneatus
Desulfovibrio desulfuricans
Desulfovibrio diazotrophicus
Desulfovibrio fairfieldensis
Desulfovibrio ferrophilus
Desulfovibrio gilichinskyi
Desulfovibrio inopinatus
Desulfovibrio intestinalis
Desulfovibrio legallii
Desulfovibrio oxyclinae
Desulfovibrio piger ATCC 29098
Desulfovibrio piger
Desulfovibrio psychrotolerans
Desulfovibrio sp. 3_1_syn3
Desulfovibrio sp. 6_1_46AFAA
Desulfovibrio sp. 86
Desulfovibrio sp. A2
Desulfovibrio sp. AM18-2
Desulfovibrio sp. An276
Desulfovibrio sp. DS-1
Desulfovibrio sp. DV
Desulfovibrio sp. Fe33
Desulfovibrio sp. JC022
Desulfovibrio sp. JY
Desulfovibrio sp. MES5
Desulfovibrio sp. TomC
Desulfovibrio sp. UIB00
Desulfovibrio sp. X2
Desulfovibrio sp.
Desulfovibrio subterraneus
Desulfovibrio sulfodismutans DSM 3696
Desulfovibrio sulfodismutans
Desulfovibrio vulgaris DP4
Desulfovibrio vulgaris RCH1
Desulfovibrio vulgaris str. Hildenborough
Desulfovibrio vulgaris
Desulfovibrio
Desulfovibrionaceae
Desulfovibrionales bacterium
Desulfovirgula thermocuniculi
Desulfovulcanus ferrireducens
Desulfurella acetivorans A63
Desulfurella multipotens
Desulfuribacillus alkaliarsenatis
Desulfuribacillus stibiiarsenatis
Desulfurispira natronophila
Desulfurispirillum indicum S5
Desulfurispirillum indicum
Desulfurispora thermophila
Desulfurivibrio alkaliphilus AHT 2
Desulfurivibrio alkaliphilus
Desulfurivibrio sp.
Desulfurobacterium atlanticum
Desulfurococcus amylolyticus
Desulfuromonadales bacterium
Desulfuromonas acetexigens
Desulfuromonas acetoxidans DSM 684
Desulfuromonas acetoxidans
Desulfuromonas soudanensis
Desulfuromonas sp. AOP6
Desulfuromonas sp. CSMB_57
Desulfuromonas sp. DDH964
Desulfuromonas sp. KJ2020
Desulfuromonas sp. SDB
Desulfuromonas sp. TF
Desulfuromonas sp.
Desulfuromonas thiophila
Desulfuromonas versatilis
Desulfuromusa kysingii
Dethiobacter alkaliphilus AHT 1
Dethiobacter alkaliphilus
Dethiobacter sp.
Dethiosulfatarculus sandiegensis
Dethiosulfatibacter aminovorans DSM 17477
Dethiosulfatibacter aminovorans
Dethiosulfovibrio faecalis
Dethiosulfovibrio peptidovorans DSM 11002
Dethiosulfovibrio peptidovorans
Dethiosulfovibrio
Devosia aurantiaca
Devosia insulae
Devosia neptuniae
Devosia oryziradicis
Devosia sediminis
Devosia sp. Leaf64
Devosia sp.
Dialister invisus CAG: 218
Dialister invisus DSM 15470
Dialister invisus
Dialister sp. CAG: 486
Dialister succinatiphilus YIT 11850
Dialister succinatiphilus
Diaphorina citri
Diaphorobacter sp. ED-3
Dichotomicrobium thermohalophilum
Dickeya chrysanthemi
Dickeya dadantii 3937
Dickeya dadantii
Dickeya dianthicola RNS04.9
Dickeya dianthicola
Dickeya fangzhongdai
Dickeya solani D s0432-1
Dickeya solani IPO 2222
Dickeya solani RNS 08.23.3.1.A
Dickeya solani
Dickeya sp. NCPPB 3274
Dickeya sp. Secpp 1600
Dickeya undicola
Dickeya zeae
Dickeya
Dictyobacter kobayashii
Dictyobacter vulcani
Dietzia aurantiaca
Dietzia cinnamea
Dietzia lutea
Dietzia maris
Dietzia massiliensis
Dietzia psychralcaliphila
Dietzia sp. E1
Dietzia sp. NCCP-2495
Dietzia sp. SLG310A2-38A2
Dietzia sp. SYD-A1
Dietzia timorensis
Dietzia
Dinoroseobacter shibae DFL 12 = DSM 16493
Dinoroseobacter shibae
Diplocloster modestus
Dissulfuribacter thermophilus
Dissulfurimicrobium hydrothermale
Dissulfurispira thermophila
Dokdonia donghaensis
Dokdonia pacifica
Dokdonia sinensis
Dokdonia sp. 4H-3-7-5
Dokdonia sp. Dokd-P16
Dokdonia sp. Hell_53
Dokdonia sp. Hel_I_63
Dokdonia sp. MED134
Dokdonia sp. PRO95
Dolichospermum circinale FSS-124
Dolichospermum circinale
Dolichospermum compactum NIES-806
Dolichospermum flos-aquae FACHB-245
Dolichospermum flos-aquae PMC207.03
Dolichospermum flos-aquae PMC208.03
Dolichospermum flos-aquae
Dolichospermum heterosporum
Dolichospermum planctonicum PMC196.03
Dolichospermum planctonicum PMC200.03
Dolichospermum planctonicum PMC230.04
Dolichospermum planctonicum
Dolichospermum sp. LEGE 00240
Dolichospermum sp. LEGE 00246
Dolichospermum sp. MN-1A
Dolichospermum sp. UHCC 0259
Dolichospermum
Domibacillus enclensis
Domibacillus indicus
Domibacillus robiginosus
Domibacillus tundrae
Donghicola eburneus
Donghicola mangrovi
Dongia mobilis
Dongshaea marina
Dorea ammoniilytica
Dorea amylophila
Dorea formicigenerans 4_6_53AFAA
Dorea formicigenerans ATCC 27755
Dorea formicigenerans CAG: 28
Dorea formicigenerans
Dorea longicatena CAG: 42
Dorea longicatena DSM 13814
Dorea longicatena
Dorea sp. 42_8
Dorea sp. AF36-15AT
Dorea sp. AM58-8
Dorea sp. CAG: 317
Dorea
Draconibacterium halophilum
Draconibacterium mangrovi
Draconibacterium orientale
Draconibacterium sediminis
Drancourtella sp. An12
Dromaius novaehollandiae
Drosophila persimilis
Duganella aceris
Duganella alba
Duganella aquatilis
Duganella callida
Duganella flavida
Duganella guangzhouensis
Duganella lactea
Duganella levis
Duganella margarita
Duganella phyllosphaerae
Duganella radicis
Duganella sacchari
Duganella sp. 1224
Duganella sp. 1411
Duganella sp. CF517
Duganella sp. CY15W
Duganella sp. FT27W
Duganella sp. Leaf126
Duganella sp. Leaf61
Duganella sp. OV458
Duganella sp. OV510
Duganella sp. SG902
Duganella sp. sic0402
Duganella vulcania
Dulcicalothrix desertica
Dyadobacter arcticus
Dyadobacter beijingensis
Dyadobacter crusticola
Dyadobacter endophyticus
Dyadobacter flavalbus
Dyadobacter jiangsuensis
Dyadobacter luteus
Dyadobacter psychrotolerans
Dyadobacter sediminis
Dyadobacter soli
Dyadobacter sp. CECT 9275
Dyadobacter sp. CECT 9623
Dyadobacter sp. CY343
Dyadobacter sp. CY357
Dyadobacter sp. Leaf189
Dyadobacter sp. SG02
Dyella jiangningensis
Dyella sp. AD56
Dysgonomonas capnocytophagoides
Dysgonomonas gadei ATCC BAA-286
Dysgonomonas gadei
Dysgonomonas mossii
Dysgonomonas sp. GY75
Dysgonomonas
Echinimonas agarilytica
Ectothiorhodosinus mongolicus
Ectothiorhodospira haloalkaliphila ATCC 51935
Ectothiorhodospira haloalkaliphila
Ectothiorhodospira magna
Ectothiorhodospira marina
Ectothiorhodospira mobilis
Ectothiorhodospira shaposhnikovii DSM 2111
Ectothiorhodospira shaposhnikovii
Ectothiorhodospira sp. BSL-9
Ectothiorhodospira sp. PHS-1
Ectothiorhodospira variabilis
Ectothiorhodospira
Ectothiorhodospiraceae bacterium BW-2
Edaphobacter dinghuensis
Edaphobacter lichenicola
Edaphobacter modestus
Edaphobacter sp. 12200R-103
Effusibacillus pohliae
Eggerthella guodeyinii
Eggerthella lenta DSM 2243
Eggerthella lenta
Eggerthella sinensis
Eggerthella sp. NSJ-70
Eggerthella timonensis
Eilatimonas milleporae
Elioraea sp. Yellowstone
Elioraea thermophila
Elizabethkingia miricola
Elizabethkingia sp. ASV34
Elizabethkingia ursingii
Elizabethkingia
Ellagibacter isourolithinifaciens
Elstera cyanobacteriorum
Elstera litoralis
Elusimicrobia bacterium
Embleya hyalina
Embleya scabrispora
Empedobacter brevis
Emticicia agri
Emticicia sp. BO119
Endobacterium cereale
Endomicrobium proavitum
Endozoicomonadaceae bacterium GTF-13
Endozoicomonas sp. SESOKO4
Enemella evansiae
Enhygromyxa salina
Ensifer adhaerens
Ensifer alkalisoli
Ensifer aridi
Ensifer glycinis
Ensifer mexicanus
Ensifer psoraleae
Ensifer sesbaniae
Ensifer shofinae
Ensifer sojae CCBAU 05684
Ensifer sojae
Ensifer sp. 1647
Ensifer sp. 1660
Ensifer sp. AC01b
Ensifer sp. AC01c1
Ensifer sp. AC01c2
Ensifer sp. AC01d1
Ensifer sp. AC01d2
Ensifer sp. AC01e
Ensifer sp. AC07a
Ensifer sp. AC07b1
Ensifer sp. AC07b2
Ensifer sp. AC07c
Ensifer sp. AC07e
Ensifer sp. AC08a1
Ensifer sp. AC08a2
Ensifer sp. AC08e
Ensifer sp. AC10a1
Ensifer sp. AC10a2
Ensifer sp. AC10d
Ensifer sp. AC11d
Ensifer sp. AC14d
Ensifer sp. AC16a
Ensifer sp. AC16b1
Ensifer sp. AC16b2
Ensifer sp. AC16c
Ensifer sp. AC17a
Ensifer sp. AC17b
Ensifer sp. AC17c
Ensifer sp. AC17d
Ensifer sp. AC17e1
Ensifer sp. AC17e2
Ensifer sp. AC18a
Ensifer sp. AC19a
Ensifer sp. AC19e
Ensifer sp. AC20b
Ensifer sp. AC21c1
Ensifer sp. AC22c
Ensifer sp. AC22d
Ensifer sp. AC22e
Ensifer sp. AC24d1
Ensifer sp. AC24d2
Ensifer sp. AC25a
Ensifer sp. AC25b
Ensifer sp. AC25c
Ensifer sp. AC25d
Ensifer sp. AC25e
Ensifer sp. AC27a1
Ensifer sp. AC27a2
Ensifer sp. AC27a3
Ensifer sp. AC27c
Ensifer sp. AC27d1
Ensifer sp. AC27d2
Ensifer sp. AC27e
Ensifer sp. AC28a
Ensifer sp. AC28d1
Ensifer sp. AC38b1
Ensifer sp. AC38b2
Ensifer sp. AC38d1
Ensifer sp. AC38d2
Ensifer sp. AC40a
Ensifer sp. AC47a
Ensifer sp. AC47b
Ensifer sp. AC47c
Ensifer sp. AC47d
Ensifer sp. AC52a1
Ensifer sp. AC52a2
Ensifer sp. AC52b
Ensifer sp. AC52c
Ensifer sp. AC52d
Ensifer sp. AC52e
Ensifer sp. ATQ1
Ensifer sp. ATQ3
Ensifer sp. BR816
Ensifer sp. CCBAU 10865
Ensifer sp. CEQ1
Ensifer sp. CHU1
Ensifer sp. CHU2
Ensifer sp. ICMP 19566
Ensifer sp. ICMP 19853
Ensifer sp. ICMP 19854
Ensifer sp. ICMP 19855
Ensifer sp. ICMP 19858
Ensifer sp. ICMP 19861
Ensifer sp. JNVU AJ10
Ensifer sp. JNVU AJ14
Ensifer sp. JNVU AJ23
Ensifer sp. JNVU AJ24
Ensifer sp. JNVU AJ31
Ensifer sp. JNVU AJ32
Ensifer sp. JNVU AL5
Ensifer sp. JNVU AN11
Ensifer sp. JNVU AN1
Ensifer sp. JNVU AN72
Ensifer sp. JNVU AN93
Ensifer sp. JNVU AS11
Ensifer sp. JNVU AS27
Ensifer sp. JNVU AS50
Ensifer sp. JNVU AS53
Ensifer sp. JNVU CB56
Ensifer sp. JNVU CP11
Ensifer sp. JNVU CP7
Ensifer sp. JNVU MH32
Ensifer sp. JNVU MH37
Ensifer sp. JNVU MH40
Ensifer sp. JNVU MH8
Ensifer sp. JNVU MHM1
Ensifer sp. JNVU MHM24
Ensifer sp. JNVU MHM40
Ensifer sp. JNVU PC16
Ensifer sp. JNVU PC17
Ensifer sp. JNVU PC2
Ensifer sp. JNVU PJ11
Ensifer sp. JNVU PJ18
Ensifer sp. JNVU PJ1
Ensifer sp. JNVU RA27
Ensifer sp. JNVU RA30
Ensifer sp. JNVU RA43
Ensifer sp. JNVU RA60
Ensifer sp. JNVU RM47
Ensifer sp. JNVU TF7
Ensifer sp. JNVU TL4
Ensifer sp. JNVU TP13
Ensifer sp. JNVU TV3
Ensifer sp. JNVU TW10
Ensifer sp. JPY1088
Ensifer sp. JPY1165
Ensifer sp. JPY1168
Ensifer sp. JPY1220
Ensifer sp. JPY851
Ensifer sp. JPY996
Ensifer sp. JPY998
Ensifer sp. LEM453
Ensifer sp. LEM456
Ensifer sp. LEM459
Ensifer sp. LEM466
Ensifer sp. LEM468
Ensifer sp. LEM551
Ensifer sp. LMR002
Ensifer sp. LMR006
Ensifer sp. LMR021
Ensifer sp. MMN_5
Ensifer sp. MPMI2T
Ensifer sp. NBAIM29
Ensifer sp. OD45
Ensifer sp. OD46
Ensifer sp. R-45905
Ensifer sp. R-46116
Ensifer sp. R-46170
Ensifer sp. R-46177
Ensifer sp. Root127
Ensifer sp. Root142
Ensifer sp. Root278
Ensifer sp. Root31
Ensifer sp. Root558
Ensifer sp. SEMIA 135
Ensifer sp. STA1
Ensifer sp. STA2
Ensifer sp. T173
Ensifer sp. WSM1721
Ensifer sp. X873B
Ensifer sp. YIC 5037
Ensifer sp. YIC 5072
Ensifer sp. YIC 5077
Ensifer sp. YIC 5090
Ensifer sp. YIC4071
Ensifer sp. YIC4108
Ensifer sp. YIC4261
Ensifer sp.
Entamoeba histolytica HM-1: IMSS-A
Entamoeba histolytica HM-1: IMSS
Entamoeba histolytica HM-3: IMSS
Entamoeba histolytica KU27
Entamoeba histolytica
Entamoeba nuttalli P19
Enteractinococcus coprophilus
Enterobacter asburiae
Enterobacter bugandensis
Enterobacter cloacae
Enterobacter hormaechei
Enterobacter ludwigii
Enterobacter quasiroggenkampii
Enterobacter sp. 10-1
Enterobacter sp. 9-2
Enterobacter sp. AG5470
Enterobacter sp. B-12
Enterobacter sp. EN-21
Enterobacter sp. FY-07
Enterobacter sp. GG165E
Enterobacter sp. GG49E
Enterobacter sp. LA14E
Enterobacter sp. LA4E
Enterobacter sp. LZ84E
Enterobacter sp. MTP_050512_17
Enterobacter sp. NFR05
Enterobacter sp. NN145S
Enterobacter sp. PG132S
Enterobacter sp. QZ25S
Enterobacter sp. QZ80E
Enterobacter sp. R4-368
Enterobacter sp. RHBSTW-00175
Enterobacter sp. Y11
Enterobacter sp. Y4
Enterobacter sp. Y61
Enterobacter sp. Y79
Enterobacter sp. YL34S
Enterobacter sp. gx-25
Enterobacter sp.
Enterobacteriaceae bacterium A-F18
Enterobacteriaceae bacterium ENNIH1
Enterobacteriaceae bacterium ENNIH2
Enterobacteriaceae bacterium ENNIH3
Enterobacteriaceae bacterium TzEc084
Enterobacteriaceae
Enterocloster aldenensis
Enterocloster bolteae 90B8
Enterocloster bolteae ATCC BAA-613
Enterocloster bolteae
Enterocloster citroniae
Enterocloster clostridioformis
Enterocloster lavalensis
Enterococcus cecorum
Enterococcus faecalis 10244
Enterococcus faecalis 918
Enterococcus faecalis BM4654
Enterococcus faecalis EnGen0064
Enterococcus faecalis EnGen0065
Enterococcus faecalis EnGen0101
Enterococcus faecalis EnGen0212
Enterococcus faecalis EnGen0235
Enterococcus faecalis EnGen0248
Enterococcus faecalis EnGen0252
Enterococcus faecalis EnGen0280
Enterococcus faecalis EnGen0294
Enterococcus faecalis EnGen0297
Enterococcus faecalis EnGen0338
Enterococcus faecalis TX0309A
Enterococcus faecalis TX0309B
Enterococcus faecalis
Enterococcus faecium
Enterococcus sp. 8G7_MSG3316
Enterococcus
Enteroscipio rubneri
Enterovibrio coralii
Enterovibrio nigricans
Enterovibrio norvegicus
Enterovibrio
Entomoplasma melaleucae
Epidermidibacterium keratini
Epilithonimonas arachidiradicis
Epilithonimonas bovis DSM 19482
Epilithonimonas bovis
Epilithonimonas caeni
Epilithonimonas hispanica
Epilithonimonas hominis
Epilithonimonas hungarica
Epilithonimonas mollis
Epilithonimonas sp. JDS
Epilithonimonas tenax
Epilithonimonas xixisoli
Epilithonimonas zeae
Epilithonimonas
Epsilonproteobacteria bacterium
Epulopiscium sp. ‘N.t. morphotype B’
Epulopiscium sp. Nele67-Bin002
Epulopiscium sp. Nele67-Bin004
Epulopiscium sp. Nele67-Bin005
Epulopiscium sp. Nuni2H_MBin001
Epulopiscium sp. SCG-B05WGA-EpuloA1
Epulopiscium sp. SCG-B11WGA-EpuloA1
Equus przewalskii
Erwinia amylovora 01SFR-BO
Erwinia amylovora ACW56400
Erwinia amylovora Ea266
Erwinia amylovora Ea356
Erwinia amylovora MR1
Erwinia amylovora NBRC 12687 = CFBP 1232
Erwinia amylovora
Erwinia iniecta
Erwinia sp. OLCASP 19
Erwinia sp. gx104
Erythranthe guttata
Erythrobacter aurantius
Erythrobacter colymbi
Erythrobacter donghaensis
Erythrobacter insulae
Erythrobacter litoralis HTCC2594
Erythrobacter litoralis
Erythrobacter longus
Erythrobacter neustonensis
Erythrobacter ramosus
Erythrobacter rubeus
Erythrobacter sp. A6_0
Erythrobacter sp. AP_23
Erythrobacter sp. BLCC-B19
Erythrobacter sp. HI0019
Erythrobacter sp. HI0028
Erythrobacter sp. HI0063
Erythrobacter sp. HI0077
Erythrobacter sp. HL-111
Erythrobacter sp. JK5
Erythrobacter sp. JL475
Erythrobacter sp. KY5
Erythrobacter sp. NAP1
Erythrobacter sp. SD-21
Erythrobacter sp. SG61-1L
Erythrobacter sp. THAF29
Erythrobacter sp. WG
Erythrobacter sp. WH131
Erythrobacter sp. YJ-T3-07
Erythrobacter tepidarius
Erythrobacteraceae bacterium HL-111
Erythrobacteraceae
Escherichia coli 908658
Escherichia coli M056
Escherichia coli
Escherichia marmotae
Escherichia phage CMSTMSU
Ethanoligenens harbinense YUAN-3
Ethanoligenens harbinense
Euanodontia ovum gill symbiont
Eubacteriaceae bacterium CHKCI004
Eubacteriaceae bacterium
Eubacteriales bacterium
Eubacteriales
Eubacterium callanderi
Eubacterium eligens CAG: 72
Eubacterium hallii CAG: 12
Eubacterium infirmum F0142
Eubacterium limosum
Eubacterium maltosivorans
Eubacterium minutum ATCC 700079
Eubacterium oxidoreducens
Eubacterium plexicaudatum
Eubacterium pyruvativorans
Eubacterium ramulus ATCC 29099
Eubacterium ramulus
Eubacterium rectale CAG: 36
Eubacterium ruminantium
Eubacterium sp. 1001713B170207_170306_E7
Eubacterium sp. 14-2
Eubacterium sp. 38_16
Eubacterium sp. AM05-23
Eubacterium sp. An11
Eubacterium sp. An3
Eubacterium sp. CAG: 146
Eubacterium sp. CAG: 248
Eubacterium sp. CAG: 251
Eubacterium sp. CAG: 252
Eubacterium sp. CAG: 274
Eubacterium sp. CAG: 38
Eubacterium sp. CAG: 76
Eubacterium sulci ATCC 35585
Eubacterium uniforme
Eubacterium xylanophilum
Eubacterium
Eucalyptus grandis
Euhalothece natronophila Z-M001
Euhalothece natronophila
Euhalothece sp. KZN 001
Euhalothece sp. T2-1114
Euryarchaeota archaeon 55_53
Euryarchaeota archaeon ADurb.Bin023
Euryarchaeota archaeon ADurb.Bin294
Euryarchaeota archaeon ADurb.BinA087
Euryarchaeota archae on SM23-78
Euryarchaeota archaeon TMED252
Euryarchaeota archaeon TMED 255
Euryarchaeota archaeon
Eurytemora affinis
Euzebya pacifica
Euzebya tangerina
Euzebyella marina
Evansella caseinilytica
Evansella cellulosilytica DSM 2522
Evansella cellulosilytica
Ewamiania thermalis TS0513
Exaiptasia diaphana
Exiguobacterium acetylicum
Exiguobacterium antarcticum B7
Exiguobacterium antarcticum
Exiguobacterium aurantiacum
Exiguobacterium enclense
Exiguobacterium indicum
Exiguobacterium mexicanum
Exiguobacterium profundum
Exiguobacterium sibiricum 255-15
Exiguobacterium sibiricum
Exiguobacterium sp. AB2
Exiguobacterium sp. AM39-5BH
Exiguobacterium sp. BMC-KP
Exiguobacterium sp. KKBO11
Exiguobacterium sp. Leaf187
Exiguobacterium sp. Leaf196
Exiguobacterium sp. MH3
Exiguobacterium sp. RIT341
Exiguobacterium sp. S17
Exiguobacterium sp. TNDT2
Exiguobacterium
Extensimonas perlucida
Faecalibacter bovis
Faecalibacterium cf. prausnitzii KLE1255
Faecalibacterium prausnitzii
Faecalibacterium sp. AF10-46
Faecalibacterium sp. AF27-11BH
Faecalibacterium sp. CAG: 82
Faecalibacterium
Faecalibaculum rodentium
Faecalicatena contorta
Falcatimonas sp. MSJ-15
Falsirhodobacter deserti
Falsirhodobacter halotolerans
Falsirhodobacter sp. 20TX0035
Falsirhodobacter sp. PG104
Falsirhodobacter sp. alg1
Falsiroseomonas algicola
Falsiroseomonas bella
Falsirose omonas frigidaquae
Falsiroseomonas selenitidurans
Falsiroseomonas stagni
Falsiroseomonas tokyonensis
Fe—Mo nitrogenase complex, dinitrogenase reductase component Arcobacter acticola
Fe—Mo nitrogenase complex, dinitrogenase reductase component Arcobacter defluvii
Fe—Mo nitrogenase complex, dinitrogenase reductase component Arcobacter ellisii
Fe—Mo nitrogenase complex, dinitrogenase reductase component Arcobacter suis CECT 7833
Fe—Mo nitrogenase complex, dinitrogenase reductase component Arcobacter venerupis
Fe—Mo nitrogenase complex, dinitrogenase reductase component Halarcobacter anaerophilus
Fe—Mo nitrogenase complex, dinitrogenase reductase component Halarcobacter ebronensis
Fe—Mo nitrogenase complex, dinitrogenase reductase component Malaciobacter molluscorum LMG 25693
Fermentimonas caenicola
Ferribacterium limneticum
Ferrigenium kumadai
Ferrimonas balearica DSM 9799
Ferriphaselus amnicola
Ferriphaselus sp. R-1
Ferroacidibacillus organovorans
Ferrovibrio sp.
Ferrovum myxofaciens
Ferruginibacter sp. HRS2-29
Fervidicella metallireducens AeB
Fervidicella metallireducens
Fervidicola ferrireducens
Fervidobacterium islandicum
Fervidobacterium
Fibrella aestuarina
Fibrobacter sp. UWB11
Fibrobacter sp. UWB12
Fibrobacter sp. UWB13
Fibrobacter sp. UWB15
Fibrobacter sp. UWB16
Fibrobacter sp. UWB1
Fibrobacter sp. UWB2
Fibrobacter sp. UWB3
Fibrobacter sp. UWB5
Fibrobacter sp. UWB6
Fibrobacter sp. UWB7
Fibrobacter sp. UWB8
Fibrobacter sp. UWH4
Fibrobacter sp. UWOV1
Fibrobacter sp. UWT2
Fibrobacter succinogenes subsp. elongatus
Fibrobacter succinogenes subsp. succinogenes S85
Fibrobacter succinogenes
Fibrobacter
Fibrobacterota bacterium
Fictibacillus arsenicus
Fictibacillus gelatini
Fictibacillus sp. FJAT-27399
Filimonas effusa
Filimonas lacunae
Filimonas zeae
Firmicutes bacterium ADurb.Bin354
Firmicutes bacterium ADurb.BinA205
Firmicutes bacterium AF12-30
Firmicutes bacterium AF36-19BH
Firmicutes bacterium AM41-11
Firmicutes bacterium CAG: 194
Firmicutes bacterium CAG: 24
Firmicutes bacterium CAG: 270
Firmicutes bacterium CAG: 466
Firmicutes bacterium CAG: 534
Firmicutes bacterium CAG: 536
Firmicutes bacterium CAG: 65
Firmicutes bacterium CAG: 882
Firmicutes bacterium CAG: 95
Firmicutes bacterium CAG_194_44_15
Firmicutes bacterium GWE2_51_13
Firmicutes bacterium GWF2_51_9
Firmicutes bacterium HGW-Firmicutes-11
Firmicutes bacterium HGW-Firmicutes-14
Firmicutes bacterium HGW-Firmicutes-15
Firmicutes bacterium HGW-Firmicutes-17
Firmicutes bacterium HGW-Firmicutes-1
Firmicutes bacterium HGW-Firmicutes-2
Firmicutes bacterium HGW-Firmicutes-3
Firmicutes bacterium HGW-Firmicutes-4
Firmicutes bacterium HGW-Firmicutes-6
Firmicutes bacterium HGW-Firmicutes-7
Firmicutes bacterium HGW-Firmicutes-8
Firmicutes bacterium HGW-Firmicutes-9
Firmicutes bacterium R50
Firmicutes bacterium
Fischerella ambigua UTEX 1903
Fischerella major
Fischerella muscicola AYJ115
Fischerella muscicola CCMEE 5323
Fischerella muscicola CG20
Fischerella muscicola SAG 1427-1 = PCC 73103
Fischerella muscicola
Fischerella sp. FACHB-380
Fischerella sp. Ind26
Fischerella sp. Ind81
Fischerella sp. LSKH
Fischerella sp. NIES-2361
Fischerella sp. NIES-3754
Fischerella sp. NIES-4106
Fischerella sp. NQAIF311
Fischerella sp. NQAIF323
Fischerella sp. PCC 9339
Fischerella sp. PCC 9431
Fischerella sp. PCC 9605
Fischerella sp.
Fischerella thermalis BR2B
Fischerella thermalis CCMEE 5194
Fischerella thermalis CCMEE 5196
Fischerella thermalis CCMEE 5198
Fischerella thermalis CCMEE 5201
Fischerella thermalis CCMEE 5205
Fischerella thermalis CCMEE 5208
Fischerella thermalis CCMEE 5268
Fischerella thermalis CCMEE 5273
Fischerella thermalis CCMEE 5282
Fischerella thermalis CCMEE 5318
Fischerella thermalis CCMEE 5319
Fischerella thermalis CCMEE 5328
Fischerella thermalis CCMEE 5330
Fischerella thermalis JSC-11
Fischerella thermalis WC217
Fischerella thermalis
Fischerella
Flammeovirga pacifica
Flammeovirga sp. MY04
Flammeovirga yaeyamensis
Flaviaesturariibacter aridisoli
Flaviaesturariibacter flavus
Flavimaricola marinus
Flavimobilis marinus
Flavimobilis soli
Flavisolibacter ginsengisoli
Flavisolibacter ginsenosidimutans
Flavisolibacter nicotianae
Flavisolibacter tropicus
Flavobacteriaceae bacterium CG17_big_fil_post_rev_8_21_14_2_50_33_15
Flavobacterium agri
Flavobacterium agrisoli
Flavobacterium alkalisoli
Flavobacterium antarcticum
Flavobacterium aquaticum
Flavobacterium aquatile
Flavobacterium aquidurense
Flavobacterium azooxidireducens
Flavobacterium beibuense
Flavobacterium bizetiae
Flavobacterium caseinilyticum
Flavobacterium cauense
Flavobacterium celericrescens
Flavobacterium cellulosilyticum
Flavobacterium cerinum
Flavobacterium cheniae
Flavobacterium cyanobacteriorum
Flavobacterium difficile
Flavobacterium endoglycinae
Flavobacterium filum
Flavobacterium franklandianum
Flavobacterium hydrophilum
Flavobacterium johnsoniae
Flavobacterium lacus
Flavobacterium limnosediminis
Flavobacterium luteum
Flavobacterium macacae
Flavobacterium macrobrachii
Flavobacterium noncentrifugens
Flavobacterium omnivorum
Flavobacterium petrolei
Flavobacterium piscinae
Flavobacterium piscis
Flavobacterium proteolyticum
Flavobacterium psychrolimnae
Flavobacterium psychrophilum
Flavobacterium psychroterrae
Flavobacterium rhamnosiphilum
Flavobacterium saliperosum
Flavobacterium sasangense
Flavobacterium silvaticum
Flavobacterium silvisoli
Flavobacterium soli
Flavobacterium sp. 11
Flavobacterium sp. 120
Flavobacterium sp. AC
Flavobacterium sp. B17
Flavobacterium sp. CG_23.5
Flavobacterium sp. CG_9.1
Flavobacterium sp. CSZ
Flavobacterium sp. F-30
Flavobacterium sp. GT3P67
Flavobacterium sp. HBTb2-11-1
Flavobacterium sp. HXWNR69
Flavobacterium sp. IMCC34775
Flavobacterium sp. J49
Flavobacterium sp. JLP
Flavobacterium sp. KBS0721
Flavobacterium sp. KJJ
Flavobacterium sp. LM4
Flavobacterium sp. Leaf82
Flavobacterium sp. MEB061
Flavobacterium sp. MFBS3-15
Flavobacterium sp. MR2016-29
Flavobacterium sp. N1719
Flavobacterium sp. N1736
Flavobacterium sp. N2270
Flavobacterium sp. RSP49
Flavobacterium sp. SLB02
Flavobacterium sp. Sd200
Flavobacterium sp. Sr18
Flavobacterium sp. TH16-21
Flavobacterium taihuense
Flavobacterium tegetincola
Flavobacterium xueshanense
Flavobacterium
Flavonifractor plautii 1_3_50AFAA
Flavonifractor plautii ATCC 29863
Flavonifractor plautii DSM 6740
Flavonifractor plautii
Flavonifractor sp. An135
Flavonifractor sp. An306
Flavonifractor sp. An92
Flexibacter sp. CG_4_10_14_3_um_filter_32_15
Flexilinea flocculi
Flexivirga aerilata
Flexivirga caeni
Flexivirga endophytica
Flexivirga oryzae
Flexivirga sp. MI-S11
Fluviicola sp. RIFCSPHIGHO2_01_FULL_43_53
Fluviicola sp. RIFCSPHIGHO2_12_FULL_43_24
Fluviispira sanaruensis
Fodinicola acaciae
Fodinicurvata sediminis
Fontibacillus panacisegetis
Fontibacillus phaseoli
Fontibacillus solani
Fontibacillus
Formivibrio citricus
Formosa agariphila
Fortiea contorta
Fortiea sp. LEGE XX443
Fragaria vesca subsp. vesca
Fragaria vesca
Francisella cf. novicida Fx1
Francisella hispaniensis FSC454
Francisella hispaniensis
Francisella opportunistica
Francisella tularensis subsp. novicida D9876
Francisella tularensis subsp. novicida GA99-3548
Francisella tularensis
Frankia alni ACN14a
Frankia alni
Frankia asymbiotica
Frankia canadensis
Frankia casuarinae
Frankia colletiae
Frankia coriariae
Frankia discariae
Frankia elaeagni
Frankia gtarii
Frankia inefficax
Frankia irregularis
Frankia saprophytica
Frankia soli
Frankia sp. 32-72
Frankia sp. 32-75
Frankia sp. 32-85
Frankia sp. 55005
Frankia sp. ACN1
Frankia sp. ACN1ag(2009)
Frankia sp. ACN1ag
Frankia sp. ARgP5_AG
Frankia sp. Ag45/Mut15
Frankia sp. Ag8c
Frankia sp. AgB16
Frankia sp. AgB20
Frankia sp. AgB32
Frankia sp. AgGS′84/18
Frankia sp. AgGS′84/44
Frankia sp. AgKG′84/4
Frankia sp. AgKG′84/5
Frankia sp. AgN2Cl1
Frankia sp. AgN2Cl2
Frankia sp. AgP1R1
Frankia sp. AgP1R2
Frankia sp. AgP1R3
Frankia sp. AgP1R4
Frankia sp. AgPm24
Frankia sp. AgW1.1
Frankia sp. Ai14a
Frankia sp. Ai7a
Frankia sp. AiBp5
Frankia sp. AiPa1
Frankia sp. AiPs1
Frankia sp. AiPs3
Frankia sp. AiPs4
Frankia sp. AirI1
Frankia sp. Allo2
Frankia sp. An2.1
Frankia sp. An2.2
Frankia sp. ArI3
Frankia sp. ArI4
Frankia sp. ArI5
Frankia sp. AvcI1
Frankia sp. AvsI4
Frankia sp. B2
Frankia sp. BCU110345
Frankia sp. BMG5.10
Frankia sp. BMG5.11
Frankia sp. BMG5.14
Frankia sp. BMG5.15
Frankia sp. BMG5.23
Frankia sp. BMG5.2
Frankia sp. BMG5.30
Frankia sp. BMG5.3
Frankia sp. BMG5.4
Frankia sp. BMG5.5
Frankia sp. BMG5.6
Frankia sp. BMG5.7
Frankia sp. CH37
Frankia sp. CIT1
Frankia sp. CNm7
Frankia sp. CcI156
Frankia sp. CcI6
Frankia sp. CeD
Frankia sp. CeF
Frankia sp. CeSI5
Frankia sp. Cg70.1
Frankia sp. CgIM4
Frankia sp. CgIS1
Frankia sp. Ch17F
Frankia sp. ChI.7
Frankia sp. CiP1_Cm_nod1
Frankia sp. CiP1_Cm_nod2
Frankia sp. CiP3
Frankia sp. CjI-82
Frankia sp. CjJ11
Frankia sp. CjJ14
Frankia sp. CmF1
Frankia sp. CmF6
Frankia sp. CmM1
Frankia sp. CmM2
Frankia sp. CpI1(2009)
Frankia sp. CpI1-P
Frankia sp. D11
Frankia sp. DC12
Frankia sp. EAN1pec
Frankia sp. EI5c
Frankia sp. EUN1f
Frankia sp. Ea1.12
Frankia sp. EuIK1
Frankia sp. FMc1
Frankia sp. FMc2
Frankia sp. FMc3
Frankia sp. FMc4
Frankia sp. FMc5
Frankia sp. FMc6
Frankia sp. FMi1
Frankia sp. FMi2
Frankia sp. FMk1
Frankia sp. FMq1
Frankia sp. HrI1F
Frankia sp. HrI1
Frankia sp. IPN Ce16
Frankia sp. KB5
Frankia sp. KB
Frankia sp. MgI5
Frankia sp. MpI1
Frankia sp. Mrp182
Frankia sp. NRRL B-16306
Frankia sp. NRRL B-16322
Frankia sp. NRRL B-16406
Frankia sp. NRRL B-16412
Frankia sp. NRRL B-16466
Frankia sp. NRRL B-16467
Frankia sp. NRRL B-16512
Frankia sp. ORS020608
Frankia sp. QA3
Frankia sp. R43(2009)
Frankia sp. R43
Frankia sp. R82
Frankia sp. R96
Frankia sp.
Frankia symbiont of Coriaria ruscifolia
Frankia tisae
Frankia torreyi
Frankia
Frankliniella occidentalis
Frateuria sp. Soil773
Frateuria terrea
Fremyella diplosiphon NIES-3275
Fretibacterium sp. OH1220_COT-178
Frigidibacter mobilis
Frigoribacterium endophyticum
Frigoribacterium faeni
Frigoribacterium sp. 9N
Frigoribacterium sp. ACAM 257
Frigoribacterium sp. CFBP 13707
Frigoribacterium sp. CFBP 13712
Frigoribacterium sp. CFBP 13729
Frigoribacterium sp. CFBP 8754
Frigoribacterium sp. CFBP 8766
Frigoribacterium sp. Leaf164
Frigoribacterium sp. Leaf172
Frigoribacterium sp. Leaf186
Frigoribacterium sp. Leaf263
Frigoribacterium sp. Leaf44
Frigoribacterium sp. Leaf8
Frigoribacterium sp. MCBA15_019
Frigoribacterium sp. MEB024
Frigoribacterium sp. NBH87
Frigoribacterium sp. PhB107
Frigoribacterium sp. PhB116
Frigoribacterium sp. PhB118
Frigoribacterium sp. PhB160
Frigoribacterium sp. PhB24
Frigoribacterium sp. PvP032
Frigoribacterium sp. PvP121
Frigoribacterium sp. RIT-PI-h
Frigoribacterium sp. SL97
Frigoribacterium sp. VKM Ac-1396
Frigoribacterium sp. VKM Ac-2530
Frigoriflavimonas asaccharolytica
Frigoriglobus tundricola
Frondihabitans australicus
Frondihabitans sp. 762G35
Frondihabitans sp. Leaf304
Frondihabitans sp. PAMC 28766
Frondihabitans sp. PhB188
Frondihabitans sp. VKM Ac-2883
Fructobacillus fructosus
Fructobacillus pseudoficulneus
Fulvimarina endophytica
Fulvimarina manganoxydans
Fulvimarina pelagi HTCC2506
Fulvimarina pelagi
Fulvivirga lutimaris
Fulvivirga sp. SW1-E11
Fundidesulfovibrio magnetotacticus
Fundide sulfovibrio putealis
Fundide sulfovibrio soli
Fundide sulfovibrio sp. SG106
Fundide sulfovibrio sp. SG127
Fusarium kuroshium
Fusarium oxysporum f. sp. cepae
Fusarium oxysporum
Fuscibacter oryzae
Fuscovulum blasticum DSM 2131
Fuscovulum blasticum
Fusibacter paucivorans
Fusicatenibacter saccharivorans
Fusicatenibacter sp. CLA-AA-H277
Fusobacterium canifelinum
Fusobacterium hwasookii ChDC F128
Fusobacterium hwasookii ChDC F174
Fusobacterium hwasookii ChDC F206
Fusobacterium hwasookii ChDC F300
Fusobacterium hwasookii
Fusobacterium massiliense
Fusobacterium nucleatum CC53
Fusobacterium nucleatum CTI-2
Fusobacterium nucleatum subsp. animalis 11_3_2
Fusobacterium nucleatum subsp. animalis 3_1_33
Fusobacterium nucleatum subsp. animalis ATCC 51191
Fusobacterium nucleatum subsp. animalis F0419
Fusobacterium nucleatum subsp. animalis
Fusobacterium nucleatum subsp. nucleatum ATCC 23726
Fusobacterium nucleatum subsp. nucleatum ATCC 25586
Fusobacterium nucleatum subsp. nucleatum ChDC F316
Fusobacterium nucleatum subsp. nucleatum
Fusobacterium nucleatum subsp. polymorphum
Fusobacterium nucleatum subsp. vincentii 3_1_27
Fusobacterium nucleatum subsp. vincentii ChDC F8
Fusobacterium nucleatum subsp. vincentii
Fusobacterium nucleatum
Fusobacterium perfoetens
Fusobacterium periodonticum D10
Fusobacterium periodonticum
Fusobacterium pseudoperiodonticum
Fusobacterium russii
Fusobacterium simiae
Fusobacterium sp. 1001295B_180824_G3
Fusobacterium sp. CAG: 649
Fusobacterium sp. CM1
Fusobacterium sp. HMSC064B12
Fusobacterium sp. HMSC073F01
Fusobacterium ulcerans 12-1B
Fusobacterium ulcerans ATCC 49185
Fusobacterium ulcerans
Fusobacterium varium ATCC 27725
Fusobacterium varium
Fusobacterium
Gaiella occulta
Gaiellales bacterium
Gallintestinimicrobium propionicum
Gallionella capsiferriformans
Gallionellaceae bacterium
Gallionellales bacterium 35-53-114
Gammaproteobacteria bacterium 28-57-27
Gammaproteobacteria bacterium BFH1
Gammaproteobacteria bacterium HGW-Gammaproteobacteria-10
Gammaproteobacteria bacterium HGW-Gammaproteobacteria-1
Gammaproteobacteria bacterium HGW-Gammaproteobacteria-4
Gammaproteobacteria bacterium MOLA455
Gammaproteobacteria bacterium SG8_11
Gammaproteobacteria bacterium SG8_47
Gammaproteobacteria bacterium TMED30
Gammaproteobacteria bacterium
Gardnerella vaginalis
Gayadomonas joobiniege
Geitlerinema sp. FC II
Geitlerinema sp. PCC 7407
Geitlerinema sp. PCC 9228
Geitlerinema sp. T6-1124
Gelidibacter sediminis
Gellertiella hungarica
Geminicoccaceae bacterium
Geminicoccus harenae
Geminicoccus roseus
Geminisphaera colitermitum
Geminocystis herdmanii
Geminocystis sp. NIES-3708
Geminocystis sp. NIES-3709
Gemmata massiliana
Gemmata obscuriglobus
Gemmata palustris
Gemmata sp. SH-PL17
Gemmatimonadetes bacterium HGW-Gemmatimonadetes-1
Gemmatimonadota bacterium
Gemmatimonas aurantiaca
Gemmatimonas groenlandica
Gemmatimonas sp. SM23 52
Gemmatirosa kalamazoonensis
Gemmiger formicilis
Gemmobacter aestuarii
Gemmobacter aquarius
Gemmobacter aquatilis
Gemmobacter fulva
Gemmobacter lanyuensis
Gemmobacter megaterium
Gemmobacter nectariphilus
Gemmobacter sp. LW-1
Gemmobacter straminiformis
Genlisea aurea
Geoalkalibacter ferrihydriticus DSM 17813
Geoalkalibacter ferrihydriticus
Geoalkalibacter halelectricus
Geoalkalibacter subterraneus
Geobacillus genomosp. 3
Geobacillus icigianus
Geobacillus kaustophilus HTA426
Geobacillus kaustophilus
Geobacillus sp. 12AMOR1
Geobacillus sp. A8
Geobacillus sp. BCO2
Geobacillus sp. C56-T3
Geobacillus sp. CAMR5420
Geobacillus sp. GHH01
Geobacillus sp. LEMMY01
Geobacillus sp. PA-3
Geobacillus sp. T6
Geobacillus sp. Y412MC52
Geobacillus sp.
Geobacillus stearothermophilus 10
Geobacillus stearothermophilus ATCC 12980
Geobacillus stearothermophilus
Geobacillus thermocatenulatus
Geobacillus thermodenitrificans NG80-2
Geobacillus thermodenitrificans
Geobacillus
Geobacter anodireducens
Geobacter argillaceus
Geobacter benzoatilyticus
Geobacter chapellei
Geobacter grbiciae
Geobacter hydrogenophilus
Geobacter luticola
Geobacter metallireducens GS-15
Geobacter metallireducens RCH3
Geobacter metallireducens
Geobacter pickeringii
Geobacter soli
Geobacter sp. AOG1
Geobacter sp. AOG2
Geobacter sp. DSM 9736
Geobacter sp. FeAm09
Geobacter sp. OR-1
Geobacter sp. SVR
Geobacter sp.
Geobacter sulfurreducens KN400
Geobacter sulfurreducens PCA
Geobacter sulfurreducens
Geobacter
Geobacteraceae bacterium
Geodermatophilus africanus
Geodermatophilus amargosae
Geodermatophilus aquaeductus
Geodermatophilus bullaregiensis
Geodermatophilus chilensis
Geodermatophilus dictyosporus
Geodermatophilus nigrescens
Geodermatophilus normandii
Geodermatophilus obscurus
Geodermatophilus poikilotrophus
Geodermatophilus pulveris
Geodermatophilus sabuli
Geodermatophilus saharensis
Geodermatophilus siccatus
Geodermatophilus sp. DF01_2
Geodermatophilus sp. DSM 45219
Geodermatophilus sp. LHW52908
Geodermatophilus sp. Leaf369
Geodermatophilus sp. TF02-6
Geodermatophilus sp. YIM 151500
Geodermatophilus telluris
Geodermatophilus tzadiensis
Geofilum rubicundum JCM 15548
Geofilum rubicundum
Geomesophilobacter sediminis
Geomicrobium sediminis
Geomicrobium sp. JCM 19038
Geomicrobium sp. JCM 19055
Geomonas agri
Geomonas anaerohicana
Geomonas azotofigens
Geomonas edaphica
Geomonas ferrireducens
Geomonas limicola
Geomonas nitrogeniifigens
Geomonas oryzae
Geomonas oryzisoli
Geomonas paludis
Geomonas propionica
Geomonas silvestris
Geomonas sp. RF6
Geomonas sp. Red32
Geomonas subterranea
Geomonas terrae
Geomonas
Geopsychrobacter electrodiphilus
Georgenia soli
Georgenia sp. EYE_87
Georgenia subflava
Geosporobacter ferrireducens
Geotalea daltonii FRC-32
Geotalea daltonii
Geotalea sp. SG 265
Geotalea toluenoxydans
Geotalea uraniireducens Rf4
Geotalea uraniireducens
Geothermobacter hydrogeniphilus
Geothrix fermentans
Geothrix sp. SG10
Geothrix sp. SG12
Geothrix sp. SG184
Geothrix sp. SG189
Geothrix sp. SG198
Geothrix sp. SG200
Geothrix sp. SG263
Geovibrio ferrireducens
Geovibrio thiophilus
Ghiorsea bivora
Gigaspora rosea
Gilliamella apicola
Gilliamella bombi
Gillisia mitskevichiae
Gillisia sp. CAL575
Gillisia sp. Hel1_33_143
Gillisia sp. Hel_I_29
Gillisia sp. Hel_I_86
Gillisia sp. M10.2A
Gilvimarinus agarilyticus
Gilvimarinus chinensis
Gilvimarinus polysaccharolyticus
Glaciecola nitratireducens FR1064
Glaciecola nitratireducens
Glaciibacter superstes
Glaciihabitans arcticus
Glaciihabitans sp. INWT7
Gloeobacter violaceus
Gloeocapsa sp. AYJ111
Gloeocapsa sp. DLM2.Bin57
Gloeocapsa sp. PCC 73106
Gloeocapsa sp. PCC 7428
Gloeocapsopsis crepidinum LEGE 06123
Gloeocapsopsis crepidinum
Gloeocapsopsis dulcis
Gloeomargarita lithophora
Gloeothece citriformis PCC 7424
Gloeothece citriformis
Gloeothece tepidariorum PCC 6909
Gloeothece verrucosa PCC 7822
Gloeothece verrucosa
Glomus cerebriforme
Glossina morsitans morsitans
Gluconacetobacter azotocaptans
Gluconacetobacter diazotrophicus PA1 5
Gluconacetobacter diazotrophicus
Gluconacetobacter dulcium
Gluconacetobacter johannae
Gluconobacter cerinus
Gluconobacter frateurii NBRC 103465
Gluconobacter japonicus
Glutamicibacter mishrai
Glutamicibacter mysorens
Glutamicibacter nicotianae
Glutamicibacter sp. BW77
Glutamicibacter sp. BW78
Glutamicibacter sp. BW80
Glutamicibacter uratoxydans
Glycine soja
Glycomyces fuscus
Goekera deserti
Golovinomyces cichoracearum
Gordonia aichiensis
Gordonia alkanivorans
Gordonia amicalis
Gordonia araii
Gordonia bronchialis
Gordonia hongkongensis
Gordonia humi
Gordonia hydrophobica
Gordonia insulae
Gordonia jinghuaigii
Gordonia jinhuaensis
Gordonia malaquae
Gordonia namibiensis
Gordonia otitidis
Gordonia paraffinivorans
Gordonia phthalatica
Gordonia polyisoprenivorans
Gordonia pseudamarae
Gordonia rubripertincta
Gordonia shandongensis
Gordonia soli
Gordonia sp. (in: high G + C Gram-positive bacteria)
Gordonia sp. ABSL49_1
Gordonia sp. C13
Gordonia sp. GONU
Gordonia sp. LAM0048
Gordonia sp. McavH-238-E
Gordonia sp. OPL2
Gordonia sp. PDNC005
Gordonia sp. SL306
Gordonia sp. SW 21
Gordonia sp. TSed Te1
Gordonia spumicola
Gordonia sputi
Gordonia tangerina
Gordonia terrae
Gordonia
Gordonibacter pamelaeae 7-10-1-b
Gordonibacter pamelaeae
Gordonibacter sp. 28C
Gordonibacter sp. An232A
Gorillibacterium massiliense
Gorillibacterium timonense
Gossypium arboreum
Gottschalkia acidurici 9a
Gottschalkia acidurici
Gottschalkia purinilytica
Gracilariopsis chorda
Gracilibacillus halophilus YIM-C55.5
Gracilibacillus halophilus
Gracilibacillus lacisalsi
Gracilibacillus massiliensis
Gracilibacter sp. BRH_c7a
Gracilimonas sp. CAU 1638
Gracilinema caldarium DSM 7334
Gracilinema caldarium
Gramella aestuarii
Gramella echinicola
Gramella flava
Gramella forsetii
Gramella fulva
Gramella lutea
Gramella portivictoriae
Gramella sabulilitoris
Gramella sediminis
Gramella sp. AN32
Gramella sp. GC03-9
Gramella sp. MAR_2010_147
Granulibacter bethesdensis
Granulicella aggregans
Granulicella sp. S156
Granulicella sp. S190
Granulicella sp. dw_53
Granulicella tundricola
Granulicoccus phenolivorans
Grimontella sp. AG753
Grimontia celer
Grimontia hollisae CIP 101886
Grimontia hollisae
Grimontia indica
Grimontia marina
Grimontia sp. AD028
Grimontia
Gryllus bimaculatus
Guillardia theta CCMP 2712
Gulbenkiania indica
Gulosibacter faecalis
Gulosibacter sp. 10
Guopingia tenuis
Gynuella sunshinyii YC6258
Gynuella sunshinyii
Hadesarchaea archaeon YNP N21
Haematobacter genomosp. 1
Haematobacter missouriensis
Haematospirillum jordaniae
Haemophilus influenzae
Hahella chejuensis KCTC 2396
Hahella chejuensis
Hahella ganghwensis
Haladaptatus cibarius
Halalkalibacillus halophilus
Halalkalibacterium ligniniphilum
Halanaerobium praevalens DSM 2228
Halanaerobium praevalens
Halanaerobium saccharolyticum subsp. saccharolyticum DSM 6643
Halanaerobium saccharolyticum
Halanaerobium salsuginis
Halanaerobium sp. T82-1
Halanaerobium sp.
Halapricum salinum
Halarchaeum acidiphilum MH1-52-1
Halarchaeum acidiphilum
Halarcobacter anaerophilus
Halarcobacter ebronensis
Haliangium sp. UPWRP_2
Haliea salexigens
Haliea sp. SAOS-164
Halieaceae bacterium MED-G27
Halieaceae bacterium
Haloactinopolyspora alba
Haloactinospora alba
Haloarcula hispanica
Haloarcula quadrata
Haloarcula sp. Atlit-120R
Haloarcula sp. Atlit-47R
Haloarcula sp. JP-Z28
Haloarcula sp. K1
Halobacillus aidingensis
Halobacillus hunanensis
Halobacillus karajensis
Halobacillus kuroshimensis
Halobacillus litoralis
Halobacillus locisalis
Halobacillus mangrovi
Halobacillus massiliensis
Halobacillus sp. BBL2006
Halobacillus sp. GSS1
Halobacillus sp. Marseille-P3879
Halobacillus trueperi
Halobacteriales archaeon QH_2_66_30
Halobacteriales archaeon QH_6_64_20
Halobacteriales archaeon QH_6_68_27
Halobacteriales archaeon QS_1_68_20
Halobacteriales archaeon QS_5_68_33
Halobacteriales archaeon QS_6_64_34
Halobacteriales archaeon SW_7_65_23
Halobacteriovorax marinus SJ
Halobacteriovorax marinus
Halobacteriovorax sp. BALOs_7
Halobacteriovorax sp. DA5
Halobacteriovorax sp. JY17
Halobacterium hubeiense
Halobacterium litoreum
Halobacterium noricense
Halobacterium salinarum
Halobacterium sp. DL1
Halobacterium sp. Gai3-17
Halobacterium sp. KA-4
Halobacterium sp. KA-6
Halobacterium sp. R2-5
Halobacterium sp. TGN-42-S1
Halobacterium sp. XZYJT26
Halobacterium
Halobacteroides halobius DSM 5150
Halobacteroides halobius
Halobaculum salinum
Halobellus sp. Atlit-31R
Halobellus sp. Atlit-38R
Halobiforma haloterrestris
Halobium salinum
Halocalculus aciditolerans
Halocella sp. SP3-1
Halochromatium glycolicum
Halochromatium roseum
Halochromatium salexigens
Halococcoides cellulosivorans
Halococcus thailandensis
Halodesulfovibrio aestuarii
Halodesulfovibrio sp. MK-HDV
Haloechinothrix alba
Haloechinothrix halophila
Haloferax sp. BAB-2207
Haloferula luteola
Halogeometricum sp. wsp3
Haloglomus sp. ZY41
Haloglomus sp. ZY58
Haloimpatiens massiliensis
Halolamina litorea
Halolamina pelagica
Halolamina sediminis
Halomicroarcula amylolytica
Halomicroarcula limicola
Halomicroarcula nitratireducens
Halomicroarcula salina
Halomicroarcula salinisoli
Halomicroarcula sp. DT1
Halomicroarcula sp. YSSS71
Halomicrobium salinisoli
Halomicronema hongdechloris
Halomicronema sp. CCY15110
Halomonas alkaliantarctica
Halomonas anticariensis
Halomonas axialensis
Halomonas campisalis
Halomonas endophytica
Halomonas hydrothermalis
Halomonas jincaotanensis
Halomonas johnsoniae
Halomonas lionensis
Halomonas lutescens
Halomonas massiliensis
Halomonas maura
Halomonas meridiana
Halomonas neptunia
Halomonas nitroreducens
Halomonas piezotolerans
Halomonas rituensis
Halomonas salicampi
Halomonas salina
Halomonas sp. 707B3
Halomonas sp. CnH100-B
Halomonas sp. DP1Y21-3
Halomonas sp. FeN2
Halomonas sp. ISL-56
Halomonas sp. M4R1S46
Halomonas sp. PBN3
Halomonas sp. PGE1
Halomonas sp. S2151
Halomonas sp. XH26
Halomonas sp.
Halomonas stenophila
Halomonas xinjiangensis
Halomonas zhuhanensis
Halomonas
Halonatronum saccharophilum
Halopelagius longus
Halopenitus persicus
Halopiger xanaduensis SH-6
Halopiger xanaduensis
Haloplanus ruber
Haloplanus sp. HW8-1
Halopolyspora algeriensis
Halopseudomonas bauzanensis
Halopseudomonas pelagia
Halopseudomonas salegens
Halopseudomonas salina
Halopseudomonas xinjiangensis
Haloquadratum walsbyi C23
Haloquadratum walsbyi
Halorhabdus utahensis
Halorhodospira abdelmalekii
Halorhodospira halochloris
Halorhodospira halophila SL1
Halorhodospira halophila
Halorhodospira neutriphila
Halorhodospira
Halorientalis litorea
Halorientalis marina
Halorientalis pallida
Halorientalis persicus
Halorientalis regularis
Halorientalis salina
Halorubrum distributum
Halorubrum ezzemoulense
Halorubrum kocurii JCM 14978
Halorubrum persicum
Halorubrum saccharovorum DSM 1137
Halorubrum salipaludis
Halorubrum sp. AJ67
Halorubrum sp. Atlit-28R
Halorubrum sp. E3
Halorubrum sp. SS7
Halorubrum tropicale
Halorubrum vacuolatum
Halorussus amylolyticus
Halorussus halobius
Halorussus litoreus
Halorussus marinus
Halorussus pelagicus
Halorussus salinus
Halorussus sp. MSC15.2
Halorussus sp. XZYJ18
Halorussus sp. XZYJT40
Halorussus sp. XZYJT49
Halorussus
Halosaccharopolyspora lacisalsi
Halosegnis sp. ZY10
Halosimplex carlsbadense
Halosimplex halophilum
Halosimplex litoreum
Halosimplex pelagicum
Halosimplex salinum
Halostagnicola sp. A56
Halostella litorea
Haloterrigena limicola JCM 13563
Haloterrigena limicola
Haloterrigena turkmenica DSM 5511
Halothece sp. MPI 96P605
Halothece sp. PCC 7418
Halothermothrix orenii H 168
Halothermothrix orenii
Halothiobacillus sp. LS2
Halotia branconii CENA186
Halotia branconii CENA390
Halotia branconii CENA392
Halotia longispora CENA184
Halotia longispora CENA420
Halotia wernerae CENA158
Halotia wernerae CENA159
Halotia wernerae CENA160
Halotia wernerae CENA391
Halovenus carboxidivorans
Halovenus salina
Halpernia humi
Hamadaea tsunoensis
Hansschlegelia beijingensis
Hansschlegelia plantiphila
Hansschlegelia quercus
Haoranjiania flava
Hapalosiphon sp. Ind23
Hapalosiphon sp. MRB220
Hapalosiphon welwitschii Ind21
Hapalosiphon welwitschii Ind22
Hapalosiphonaceae cyanobacterium JJU2
Hapalosiphonaceae
Harryflintia acetispora
Hartmannibacter diazotrophicus
Hasllibacter halocynthiae
Hassalia byssoidea
Hathewaya massiliensis
Helianthus annuus
Helicobacter anseris
Helicobacter apodemus
Helicobacter cetorum MIT 00-7128
Helicobacter cetorum MIT 99-5656
Helicobacter cetorum
Helicobacter heilmannii
Helicobacter hepaticus ATCC 51449
Helicobacter hepaticus
Helicobacter mesocricetorum
Helicobacter pametensis
Helicobacter pylori 52
Helicobacter pylori Aklavik86
Helicobacter pylori BM012A
Helicobacter pylori BM012S
Helicobacter pylori CCHI 33
Helicobacter pylori CPY1662
Helicobacter pylori CPY3281
Helicobacter pylori CPY6261
Helicobacter pylori CPY6271
Helicobacter pylori ELS37
Helicobacter pylori F30
Helicobacter pylori FD423
Helicobacter pylori FD430
Helicobacter pylori FD506
Helicobacter pylori FD568
Helicobacter pylori FD577
Helicobacter pylori FD662
Helicobacter pylori FD703
Helicobacter pylori GAM101Biv
Helicobacter pylori GAM114Ai
Helicobacter pylori GAM119Bi
Helicobacter pylori GAM120Ai
Helicobacter pylori GAM231Ai
Helicobacter pylori GAM239Bi
Helicobacter pylori GAM245Ai
Helicobacter pylori GAM249T
Helicobacter pylori GAM250AFi
Helicobacter pylori GAM250T
Helicobacter pylori GAM252Bi
Helicobacter pylori GAM252T
Helicobacter pylori GAM254Ai
Helicobacter pylori GAM260BSi
Helicobacter pylori GAM270ASi
Helicobacter pylori GAM71Ai
Helicobacter pylori GAM93Bi
Helicobacter pylori GC26
Helicobacter pylori HP116Bi
Helicobacter pylori HP250AFiV
Helicobacter pylori HP250AFii
Helicobacter pylori HP250AFiii
Helicobacter pylori HP250ASi
Helicobacter pylori HP250ASii
Helicobacter pylori HP250BFiV
Helicobacter pylori HP250BFi
Helicobacter pylori HP250BFii
Helicobacter pylori HP250BFiii
Helicobacter pylori HP250BSi
Helicobacter pylori HP260BFii
Helicobacter pylori HPAG1
Helicobacter pylori HUP-B14
Helicobacter pylori Hp A-11
Helicobacter pylori Hp A-14
Helicobacter pylori Hp A-17
Helicobacter pylori Hp A-27
Helicobacter pylori Hp A-5
Helicobacter pylori Hp A-8
Helicobacter pylori Hp A-9
Helicobacter pylori Hp H-10
Helicobacter pylori Hp H-1
Helicobacter pylori Hp H-21
Helicobacter pylori Hp H-30
Helicobacter pylori Hp H-36
Helicobacter pylori Hp H-42
Helicobacter pylori Hp H-4
Helicobacter pylori Hp H-5b
Helicobacter pylori Hp H-9
Helicobacter pylori Hp P-13
Helicobacter pylori Hp P-13b
Helicobacter pylori Hp P-1
Helicobacter pylori Hp P-1b
Helicobacter pylori Hp P-26
Helicobacter pylori Hp P-2
Helicobacter pylori Hp P-2b
Helicobacter pylori Hp P-30
Helicobacter pylori Hp P-3
Helicobacter pylori Hp P-3b
Helicobacter pylori Hp P-41
Helicobacter pylori Hp P-62
Helicobacter pylori Lithuania75
Helicobacter pylori NQ4053
Helicobacter pylori NQ4076
Helicobacter pylori NQ4099
Helicobacter pylori NQ4200
Helicobacter pylori OK113
Helicobacter pylori OK310
Helicobacter pylori PZ5004
Helicobacter pylori PZ5024
Helicobacter pylori PZ5026
Helicobacter pylori PZ5080
Helicobacter pylori PZ5086
Helicobacter pylori PeCan18
Helicobacter pylori PeCan4
Helicobacter pylori Puno120
Helicobacter pylori Puno135
Helicobacter pylori R030b
Helicobacter pylori R036d
Helicobacter pylori R038b
Helicobacter pylori R046Wa
Helicobacter pylori R055a
Helicobacter pylori R32b
Helicobacter pylori SNT49
Helicobacter pylori Shi112
Helicobacter pylori SouthAfrica20
Helicobacter pylori SouthAfrica50
Helicobacter pylori UM023
Helicobacter pylori UM038
Helicobacter pylori UM066
Helicobacter pylori UM077
Helicobacter pylori UM084
Helicobacter pylori UM111
Helicobacter pylori UM114
Helicobacter pylori UMB_G1
Helicobacter pylori oki102
Helicobacter pylori oki422
Helicobacter pylori oki898
Helicobacter pylori v225d
Helicobacter pylori
Helicobacter sp. 12S02232-10
Helicobacter sp. 15-1451
Helicobacter sp. MIT 14-3879
Helicobacter sp. MIT 99-5507
Helicocarpus griseus UAMH5409
Heliobacterium chlorum
Heliobacterium mobile
Heliobacterium modesticaldum
Heliobacterium
Heliomicrobium gestii
Heliomicrobium modesticaldum Ice1
Heliomicrobium modesticaldum
Heliomicrobium undosum
Heliophilum fasciatum
Heliorestis acidaminivorans
Heliorestis baculata
Heliorestis convoluta
Heliorestis daurensis
Hellea balneolensis
Henriciella algicola
Henriciella barbarensis
Henriciella litoralis
Henriciella marina
Herbaspirillum aquaticum
Herbaspirillum camelliae
Herbaspirillum frisingense GSF30
Herbaspirillum frisingense
Herbaspirillum huttiense
Herbaspirillum robiniae
Herbaspirillum rubrisubalbicans M1
Herbaspirillum rubrisubalbicans Os34
Herbaspirillum rubrisubalbicans
Herbaspirillum seropedicae SmR1
Herbaspirillum seropedicae
Herbaspirillum sp. 1130
Herbaspirillum sp. AP02
Herbaspirillum sp. ASV7
Herbaspirillum sp. B39
Herbaspirillum sp. B501
Herbaspirillum sp. B65
Herbaspirillum sp. BH-1
Herbaspirillum sp. C7C2
Herbaspirillum sp. C7C8
Herbaspirillum sp. C9C3
Herbaspirillum sp. CAH-3
Herbaspirillum sp. GW103
Herbaspirillum sp. HC18
Herbaspirillum sp. KP1-50
Herbaspirillum sp. KP1-77
Herbaspirillum sp. LeCh32-8
Herbaspirillum sp. REICA_064
Herbaspirillum sp. SJZ107
Herbaspirillum sp. ST 5-3
Herbaspirillum sp. VT-16-41
Herbaspirillum sp. WGmk3
Herbaspirillum sp. YR522
Herbaspirillum sp. alder98
Herbaspirillum sp. meg3
Herbaspirillum
Herbiconiux flava
Herbiconiux ginsengi
Herbiconiux moechotypicola
Herbiconiux solani
Herbiconiux sp. CPCC 205763
Herbiconiux sp. L3-i23
Herbiconiux sp. SALV-R1
Herbiconiux sp. VKM Ac-1786
Herbidospora cretacea
Herbidospora daliensis
Herbidospora yilanensis
Herbinix hemicellulosilytica
Herbinix luporum
Herminiimonas arsenitoxidans
Herminiimonas contaminans
Herminiimonas sp. CN
Herpetosiphon geysericola
Herpetosiphon giganteus
Herpetosiphon llansteffanensis
Heterocapsa triquetra
Heteroscytonema crispum BRCC0217
Heteroscytonema crispum BRSC0217
Heteroscytonema crispum EC0914.03
Heteroscytonema crispum EC1014.03
Heteroscytonema crispum EC1014.05
Heteroscytonema crispum EC1014.07
Heteroscytonema crispum EC1014.09
Heteroscytonema crispum SSC1014.02
Heteroscytonema crispum UCFS10
Heteroscytonema crispum UCFS15
Heteroscytonema crispum UTEX LB 1556
Heteroscytonema crispum WC0914.04
Heteroscytonema crispum WC0914.05
Heteroscytonema crispum WC0914.06
Heteroscytonema crispum YC0316
Heyndrickxia sporothermodurans
Hippea alviniae
Hippea jasoniae
Hippea maritima DSM 10411
Hippea maritima
Hippea sp. KM1
Hirschia maritima
Hoeflea sp. E7-10
Hoeflea sp. WL0058
Holophaga foetida
Hominimerdicola aceti
Hugenholtzia roseola
Huintestinicola butyrica
Humibacillus sp. DSM 29435
Humibacter sp. RRB41
Humidesulfovibrio mexicanus
Humisphaera borealis
Hungatella hathewayi DSM 13479
Hungatella hathewayi
Hungatella xylanolytica
Hungatella
Hwanghaeella grinnelliae
Hwanghaeicola aestuarii
Hyalangium minutum
Hyalangium sp. H56D21
Hydrocarboniphaga effusa AP103
Hydrococcus rivularis
Hydrocoleum lyngbyaceum IZ
Hydrocoleum sp. CS-953
Hydrocoleum sp. MF1
Hydrocoryne sp. CENA393
Hydrocoryne sp. CENA398
Hydrogenibacillus schlegelii
Hydrogenimonas thermophila
Hydrogenispora ethanolica
Hydrogenivirga sp. 128-5-R1-1
Hydrogenobacter hydrogenophilus
Hydrogenobacter sp.
Hydrogenobacter thermophilus TK-6
Hydrogenobacter thermophilus
Hydrogenophaga aromaticivorans
Hydrogenophaga crocea
Hydrogenophaga electricum
Hydrogenophaga flava
Hydrogenophaga intermedia
Hydrogenophaga sp. A37
Hydrogenophaga sp. IBVHS1
Hydrogenophaga sp. IBVHS2
Hydrogenophaga sp. NFH-34
Hydrogenophaga sp. RAC07
Hydrogenophaga sp. SL48
Hydrogenophaga taeniospiralis
Hydrogenophaga
Hydrogenophilales bacterium 12-63-5
Hydrogenophilales bacterium 16-64-40
Hydrogenophilales bacterium 17-61-76
Hydrogenophilales bacterium 17-64-11
Hydrogenophilales bacterium 17-64-65
Hydrogenophilales bacterium 28-61-11
Hydrogenophilales bacterium 28-61-23
Hydrogenophilales bacterium CG17_big_fil_post_rev_8_21_14_2_50_63_12
Hydrogenophilales bacterium CG18_big_fil_WC_8_21_14_2_50_58_12
Hydrogenophilales bacterium CG_4_10_14_3_um_filter_58_23
Hydrogenophilales bacterium CG_4_10_14_3_um_filter_63_21
Hydrogenophilales bacterium CG_4_9_14_3_um_filter_59_35
Hydrogenophilales bacterium CG_4_9_14_3_um_filter_63_34
Hydrogenophilales bacterium RIFOXYD1_FULL_62_11
Hydrogenothermus marinus
Hydrogenovibrio halophilus
Hydrogenovibrio kuenenii
Hyella patelloides LEGE 07179
Hyella patelloides
Hylemonella gracilis ATCC 19624
Hylemonella gracilis str. Niagara R
Hylemonella gracilis
Hymenobacter lapidarius
Hymenobacter mucosus
Hymenobacter nivis
Hymenobacter perfusus
Hymenobacter psychrotolerans
Hymenobacter rigui
Hymenobacter ruricola
Hymenobacter sp. 15J16-1T3B
Hymenobacter sp. 5420S-77
Hymenobacter sp. AT01-02
Hymenobacter sp. BRD67
Hymenobacter sp. BT523
Hymenobacter sp. BT635
Hymenobacter sp. BT728
Hymenobacter sp. CRA2
Hymenobacter sp. HSC-4F20
Hymenobacter sp. YIM 151500-1
Hymenobacter sp. YIM 151858-1
Hymenobacter swuensis
Hyphomicrobiaceae bacterium TMED74
Hyphomicrobiales bacterium
Hyphomicrobiales
Hyphomicrobium denitrificans
Hyphomicrobium sp. 12-62-95
Hyphomicrobium sp. 32-62-53
Hyphomicrobium sp. GJ21
Hyphomicrobium sp. MC1
Hyphomicrobium sp. NDB2Meth4
Hyphomicrobium sp.
Hyphomonadaceae bacterium BRH_c29
Hyphomonadaceae bacterium UKL13-1
Hyphomonas chukchiensis
Hyphomonas pacifica
Hyphomonas sp. 34-62-18
Hyphomonas sp. BRH_c22
Ideonella alba
Ideonella aquatica
Ideonella azotifigens
Ideonella benzenivorans
Ideonella dechloratans
Ideonella sakaiensis
Ideonella sp. A 288
Ideonella sp. B508-1
Ideonella sp. Long 7
Ideonella sp. NS12-5
Idiomarina baltica OS145
Idiomarina baltica
Idiomarina loihiensis
Idiomarina sp. FeN1
Idiomarina sp. H105
Idiomarina sp. T82-3
Idiomarina sp. WRN-38
Idiomarina xiamenensis 10-D-4
Idiomarina xiamenensis
Idiomarina
Idiomarinaceae bacterium HL-53
Ignavibacteriae bacterium HGW-Ignavibacteriae-2
Ignavibacteriae bacterium HGW-Ignavibacteriae-3
Ignavibacteriales bacterium CG18_big_fil_WC_8_21_14_2_50_31_20
Ignavibacteriales bacterium
Ilyobacter polytropus DSM 2926
Ilyobacter polytropus
Imhoffiella purpurea
Immundisolibacter cernigliae
Indiicoccus explosivorum
Indioceanicola profundi
Inediibacterium massiliense
Inhella gelatinilytica
Iningainema pulvinus ES0614
Iningainema tapete
Insolitispirillum peregrinum
Intestinibacter bartlettii DSM 16795
Intestinibacter bartlettii
Intestinibaculum porci
Intestinimonas butyriciproducens
Intestinimonas timonensis
Intrasporangium calvum
Intrasporangium chromatireducens
Intrasporangium oryzae NRRL B-24470
Intrasporangium oryzae
Iocasia fonsfrigidae
Iodidimonas gelatinilytica
Iodobacter sp. BJB302
Isoptericola chiayiensis
Isoptericola halotolerans
Isoptericola sediminis
Isoptericola sp. BMS4
Isoptericola variabilis
Janibacter alkaliphilus
Janibacter anophelis
Janibacter indicus
Janibacter limosus
Janibacter sp. CX7
Janibacter sp. YB324
Jannaschia aquimarina
Jannaschia faecimaris
Jannaschia pohangensis
Jannaschia seohaensis
Jannaschia sp. M317
Jannaschia sp. Os4
Janthinobacterium agaricidamnosum
Janthinobacterium lividum
Janthinobacterium psychrotolerans
Janthinobacterium sp. 35
Janthinobacterium sp. 61
Janthinobacterium sp. 64
Janthinobacterium sp. 67
Janthinobacterium sp. 78
Janthinobacterium sp. AD80
Janthinobacterium sp. Ant5-2-1
Janthinobacterium sp. BJB301
Janthinobacterium sp. BJB303
Janthinobacterium sp. BJB412
Janthinobacterium sp. BJB446
Janthinobacterium sp. FT14W
Janthinobacterium sp. FT58W
Janthinobacterium sp. GW458P
Janthinobacterium sp. HH104
Janthinobacterium sp. LM6
Janthinobacterium sp. OK676
Janthinobacterium sp. PC23-8
Janthinobacterium sp. RA13
Janthinobacterium sp. YR213
Janthinobacterium sp. hw3
Janthinobacterium svalbardensis
Janthinobacterium tructae
Jatropha curcas
Jatrophihabitans endophyticus
Jatrophihabitans sp. GAS493
Jatrophihabitans telluris
Jejuia pallidilutea
Jeotgalibacillus campisalis
Jeotgalibacillus malaysiensis
Jeotgalibacillus proteolyticus
Jeotgalibacillus soli
Jiangella alba
Jiangella alkaliphila
Jiangella anatolica
Jiangella asiatica
Jiangella aurantiaca
Jiangella endophytica
Jiangella mangrovi
Jiangella muralis
Jiangella rhizosphaerae
Jiangella sp. DSM 45060
Jiangella ureilytica
Jidongwangia harbinensis
Jiella sp. R10
Jongsikchunia kroppenstedtii
Kaistella antarctica
Kaistella carnis
Kaistella soli
Kaistella sp. GW4-15
Kaistia terrae
Kamptonema animale
Kamptonema formosum
Kamptonema sp. PCC 6506
Kamptonema
Kandeliimicrobium roseum
Kangiella sp.
Kangsaoukella pontilimi
Katagnymene spiralis
Ketobacter sp. GenoA1
Ketobacter sp. MCCC 1A13808
Ketobacter sp.
Kibdelosporangium aridum
Kibdelosporangium philippinense
Kibdelosporangium phytohabitans
Kibdelosporangium sp. MJ126-NF4
Kiloniella laminariae
Kiloniella litopenaei
Kiloniella majae
Kiloniella sp. EL199
Kiloniella spongiae
Kineococcus aurantiacus
Kineococcus indalonis
Kineococcus radiotolerans
Kineococcus rhizosphaerae
Kineococcus rubinsiae
Kineococcus siccus
Kineococcus sp. TRM81007
Kineococcus vitellinus
Kineococcus xinjiangensis
Kineosporia corallincola
Kineosporia rhizophila
Kineosporia sp. A_224
Kineosporia sp. R_H_3
Kineothrix alysoides
Kinneretia asaccharophila
Kitasatospora albolonga
Kitasatospora aureofaciens
Kitasatospora cheerisanensis
Kitasatospora cineracea
Kitasatospora griseola
Kitasatospora niigatensis
Kitasatospora paracochleata
Kitasatospora phosalacinea
Kitasatospora purpeofusca
Kitasatospora sp. A2-31
Kitasatospora sp. CB01950
Kitasatospora sp. CB02891
Kitasatospora sp. DSM 101779
Kitasatospora sp. HUAS 3-15
Kitasatospora sp. MBT63
Kitasatospora sp. MBT66
Kitasatospora sp. MMS16-BH015
Kitasatospora sp. MY 5-36
Kitasatospora sp. NA04385
Kitasatospora sp. NBC_00240
Kitasatospora sp. NBC_01287
Kitasatospora sp. RG8
Kitasatospora sp. Root107
Kitasatospora sp. SUK 42
Kitasatospora sp. SolWspMP-SS2h
Kitasatospora sp. YST-16
Kitasatospora xanthocidica
Kitasatospora
Klebsiella aerogenes
Klebsiella grimontii
Klebsiella indica
Klebsiella michiganensis E718
Klebsiella michiganensis HKOPL1
Klebsiella michiganensis KCTC 1686
Klebsiella michiganensis
Klebsiella oxytoca KONIH1
Klebsiella oxytoca
Klebsiella pasteurii
Klebsiella pneumoniae 342
Klebsiella pneumoniae subsp. pneumoniae CIP 52.145 = B5055
Klebsiella pneumoniae subsp. pneumoniae
Klebsiella pneumoniae subsp. rhinoscleromatis
Klebsiella pneumoniae
Klebsiella quasipneumoniae subsp. quasipneumoniae
Klebsiella quasipneumoniae subsp. similipneumoniae
Klebsiella quasipneumoniae
Klebsiella quasivariicola
Klebsiella sp. 2680
Klebsiella sp. 54
Klebsiella sp. A-Nf5
Klebsiella sp. AF-4C
Klebsiella sp. AL050511_01
Klebsiella sp. AL060224_03
Klebsiella sp. AL060225_04
Klebsiella sp. AS10
Klebsiella sp. B-Nf7
Klebsiella sp. BDA134-6
Klebsiella sp. BM92
Klebsiella sp. BN-4A
Klebsiella sp. C-Nf10
Klebsiella sp. C1-16S-Nf17
Klebsiella sp. CRLI0713
Klebsiella sp. CRLI0715
Klebsiella sp. CRLI0718a
Klebsiella sp. CRLI0728
Klebsiella sp. CRLI0730
Klebsiella sp. CRLS0617
Klebsiella sp. CRLS064
Klebsiella sp. CRLS069a
Klebsiella sp. CRPV0610
Klebsiella sp. CRPV0611a
Klebsiella sp. CRRI-90_SW1
Klebsiella sp. CRRI-SW12
Klebsiella sp. CTHL.F3a
Klebsiella sp. CVUAS 10191.3
Klebsiella sp. CVUAS 5466.2
Klebsiella sp. CZ150S
Klebsiella sp. CZ186S
Klebsiella sp. D-Nf1
Klebsiella sp. DX194E
Klebsiella sp. E-Nf3
Klebsiella sp. ECI-10A
Klebsiella sp. F-Nf9
Klebsiella sp. FDAARGOS_511
Klebsiella sp. G-Nf4
Klebsiella sp. G2-16S-Nf13
Klebsiella sp. GG160E
Klebsiella sp. GG164S
Klebsiella sp. GG41E
Klebsiella sp. GG42E
Klebsiella sp. GG50E
Klebsiella sp. GG53E
Klebsiella sp. GL120222-02
Klebsiella sp. Gad1
Klebsiella sp. Gez1
Klebsiella sp. H-Nf2
Klebsiella sp. HL01
Klebsiella sp. HMSC16C06
Klebsiella sp. HMSC22F09
Klebsiella sp. HMSC25G12
Klebsiella sp. HSTU-Sny5
Klebsiella sp. HX149S
Klebsiella sp. I-Nf8
Klebsiella sp. J-Nf11
Klebsiella sp. JT42
Klebsiella sp. K-Nf6
Klebsiella sp. K5
Klebsiella sp. KG9
Klebsiella sp. KTE92
Klebsiella sp. Kd70 TUC-EEAOC
Klebsiella sp. LA11E
Klebsiella sp. LA16S
Klebsiella sp. LA3E
Klebsiella sp. LC55S
Klebsiella sp. LC89E
Klebsiella sp. LTGPAF-6F
Klebsiella sp. LZ83E
Klebsiella sp. LZ87E
Klebsiella sp. M5al
Klebsiella sp. MBT K-1
Klebsiella sp. ME-303
Klebsiella sp. MNFG_801b
Klebsiella sp. MNFG_802
Klebsiella sp. MNS_801a
Klebsiella sp. MNS_801b
Klebsiella sp. MNW_801b
Klebsiella sp. MPUS7
Klebsiella sp. MS 92-3
Klebsiella sp. NN169S
Klebsiella sp. NTI31
Klebsiella sp. OBRC7
Klebsiella sp. Obi1
Klebsiella sp. P0638
Klebsiella sp. P0640
Klebsiella sp. P0643
Klebsiella sp. P0646
Klebsiella sp. P1CD1
Klebsiella sp. PCX
Klebsiella sp. PG122E
Klebsiella sp. PL-2018
Klebsiella sp. PO552
Klebsiella sp. QZ33S
Klebsiella sp. RCJ4
Klebsiella sp. REICA_034
Klebsiella sp. SS107E
Klebsiella sp. SS82E
Klebsiella sp. T11
Klebsiella sp. TD153S
Klebsiella sp. TT001
Klebsiella sp. WR8
Klebsiella sp. X1-16S-Nf21
Klebsiella sp. Y57
Klebsiella sp. Y83
Klebsiella sp. YX115S
Klebsiella sp. YX116S
Klebsiella sp. YX117S
Klebsiella sp. YX118S
Klebsiella sp. enrichment culture clone AW1
Klebsiella sp. enrichment culture clone W2
Klebsiella sp. gx-3
Klebsiella sp.
Klebsiella spallanzanii
Klebsiella variicola At-22
Klebsiella variicola CAG: 634
Klebsiella variicola subsp. tropica
Klebsiella variicola subsp. variicola
Klebsiella variicola
Klebsiella
Klebsormidium nitens
Klenkia brasiliensis
Klenkia marina
Klenkia soli
Klenkia sp. PcliD-1-E
Klenkia taihuensis
Klenkia terrae
Kluyvera ascorbata
Kluyvera intermedia
Kluyvera sp. Nf5
Knoellia sp. DB2414S
Kocuria coralli
Kocuria dechangensis
Kocuria flava
Kocuria indica
Kocuria polaris
Kocuria rhizophila DC2201
Kocuria rhizophila
Kocuria rosea
Kocuria salina
Kocuria sediminis
Kocuria soli
Kocuria sp. BT304
Kocuria sp. CCBAU 10913
Kocuria sp. CCUG 69068
Kocuria sp. CNJ-770
Kocuria sp. JC486
Kocuria sp. LUK
Kocuria sp. SM24M-10
Kocuria sp. UCD-OTCP
Kocuria sp. WN036
Kocuria turfanensis
Komagataeibacter kakiaceti
Komagataeibacter melaceti
Komagataeibacter melomenusus
Komagataeibacter nataicola
Komagataeibacter sp. FNDCF1
Komagataeibacter sp. FNDCR2
Komagataeibacter swingsii
Komagataeibacter xylinus NBRC 13693
Komagataeibacter xylinus
Komarekiella sp. ‘clone 1’
Konateibacter massiliensis
Kordia algicida
Kordia zhangzhouensis
Kordiimonadales bacterium
Kosakonia arachidis
Kosakonia oryzae
Kosakonia oryzendophytica
Kosakonia oryziphila
Kosakonia pseudosacchari
Kosakonia quasisacchari
Kosakonia radicincitans DSM 16656
Kosakonia radicincitans UMEnt01/12
Kosakonia radicincitans YD4
Kosakonia radicincitans
Kosakonia sacchari SP1
Kosakonia sacchari
Kosakonia sp. AG348
Kosakonia sp. H7A
Kosakonia sp. LAM2021
Kosakonia sp. MH5
Kosakonia sp. ML.JS2a
Kosakonia sp. MUSA4
Kosakonia sp. R1.Fl
Kosakonia sp. SOY2
Kosakonia
Kosmotoga arenicorallina
Kosmotoga olearia TBF 19.5.1
Kosmotoga olearia
Kosmotoga pacifica
Kosmotoga
Kouleothrix aurantiaca
Kovacikia minuta
Kozakia baliensis
Krasilnikovia cinnamomea
Krasilnikoviella flava
Kribbella amoyensis
Kribbella capetownensis
Kribbella jiaozuonensis
Kribbella pittospori
Kribbella shirazensis
Kribbella soli
Kribbella sp. CA-293567
Kribbella sp. VKM Ac-2566
Kribbella sp. VKM Ac-2568
Kribbella sp. VKM Ac-2570
Kribbella sp. VKM Ac-2572
Kribbella speibonae
Kroppenstedtia eburnea
Kryptousia microlepis CENA354
Ktedonobacter sp. SOSP1-52
Ktedonospora formicarum
Ktedonosporobacter rubrisoli
Kurthia huakuii
Kurthia massiliensis
Kurthia sp. 11kri321
Kurthia sp. Dielmo
Kyrpidia spormannii
Kyrpidia tusciae DSM 2912
Kyrpidia tusciae
Labedaea rhizosphaerae
Labilibacter marinus
Labilibacter sediminis
Labilibaculum antarcticum
Labilibaculum filiforme
Labilibaculum
Labilithrix luteola
Labrys sp. KNU-23
Labrys sp. WJW
Labrys
Laccaria amethystina LaAM-08-1
Laceyella sediminis
Lachnobacterium bovis DSM 14045
Lachnobacterium bovis
Lachnoclostridium pacaense
Lachnoclostridium phytofermentans ISDg
Lachnoclostridium phytofermentans
Lachnoclostridium sp. 210928-DFI.6.3
Lachnoclostridium sp. YL32
Lachnospira eligens
Lachnospira multipara
Lachnospiraceae bacterium 10-1
Lachnospiraceae bacterium 28-4
Lachnospiraceae bacterium 3-1
Lachnospiraceae bacterium 7_1_58FAA
Lachnospiraceae bacterium A2
Lachnospiraceae bacterium A4
Lachnospiraceae bacterium C10
Lachnospiraceae bacterium G11
Lachnospiraceae bacterium G41
Lachnospiraceae bacterium JC7
Lachnospiraceae bacterium KHIT2
Lachnospiraceae bacterium KHCPX20
Lachnospiraceae bacterium NE2001
Lachnospiraceae bacterium NK3A20
Lachnospiraceae bacterium OF09-33XD
Lachnospiraceae bacterium RM5
Lachnospiraceae bacterium TWA4
Lachnospiraceae bacterium XPB1003
Lachnospiraceae bacterium
Lachnospiraceae
Lachnotalea glycerini
Lacibacter cauensis
Lacibacter luteus
Lacibacter sp. S13-6-22
Lacimicrobium alkaliphilum
Lacisediminihabitans changchengi
Lacisediminihabitans profunda
Lacrimispora aerotolerans
Lacrimispora algidixylanolytica
Lacrimispora amygdalina
Lacrimispora celerecrescens
Lacrimispora indolis
Lacrimispora sphenoides
Lacrimispora xylanolytica
Lacrimispora
Lacticaseibacillus rhamnosus
Lactiplantibacillus plantarum
Lactococcus hodotermopsidis
Lactococcus lactis
Lactococcus termiticola
Lactonifactor longoviformis DSM 17459
Lactonifactor longoviformis
Lactonifactor
Lamprobacter modestohalophilus
Lamprocystis purpurea
Lapillicoccus jejuensis
Laribacter hongkongensis
Laspinema sp. D2d
Laspinema sp. D3a
Laspinema sp. D3b
Laspinema sp. D3c
Laspinema sp. D3d
Latescibacteria bacterium DG_63
Leadbettera azotonutricia ZAS-9
Leadbettera azotonutricia
Lebetimonas sp. JS032
Leclercia adecarboxylata
Lederbergia galactosidilytica
Lederbergia lenta
Leeia aquatica
Leeia sp. TBRC 13508
Leekyejoonella antrihumi
Leeuwenhoekiella aequorea
Leeuwenhoekiella aestuarii
Leeuwenhoekiella blandensis
Leeuwenhoekiella marinoflava
Leeuwenhoekiella palythoae
Leeuwenhoekiella polynyae
Leeuwenhoekiella sp. MAR_2009_132
Leeuwenhoekiella sp. Mr9
Leeuwenhoekiella sp. ZYFB001
Legionella birminghamensis
Legionella brunensis
Legionella cherrii
Legionella drancourtii LLAP12
Legionella drancourtii
Legionella erythra
Legionella fallonii LLAP-10
Legionella fallonii
Legionella feeleii
Legionella geestiana
Legionella jamestowniensis
Legionella massiliensis
Legionella moravica
Legionella nautarum
Legionella norrlandica
Legionella pneumophila
Legionella quinlivanii
Legionella rowbothamii
Legionella sainthelensi
Legionella santicrucis
Legionella saoudiensis
Legionella shakespearei DSM 23087
Legionella shakespearei
Legionella steelei
Legionella tucsonensis
Legionella worsleiensis
Leifsonia flava
Leifsonia naganoensis
Leifsonia shinshuensis
Leifsonia sp. AK011
Leifsonia sp. NCR5
Leifsonia sp. PS1209
Leifsonia sp. TF02-11
Leisingera caerulea
Leisingera sp. ANG-DT
Leisingera sp. ANG-M7
Lelliottia amnigena
Lelliottia nimipressuralis
Lentilactobacillus hilgardii
Lentimicrobium saccharophilum
Lentimonas sp. CC10
Lentimonas sp. CC11
Lentimonas sp. CC19
Lentimonas sp. CC21
Lentimonas sp. CC4
Lentimonas sp. CC6
Lentimonas sp. CC8
Lentisphaera araneosa
Lentisphaerota bacterium
Lentzea aerocolonigenes
Lentzea alba
Lentzea albida
Lentzea albidocapillata
Lentzea atacamensis
Lentzea californiensis
Lentzea cavernae
Lentzea flava
Lentzea flaviverrucosa
Lentzea fradiae
Lentzea guizhouensis
Lentzea indica
Lentzea jiangxiensis
Lentzea kentuckyensis
Lentzea nigeriaca
Lentzea pudingi
Lentzea sp. CC55
Lentzea sp. HUAS12
Lentzea sp. NEAU-D7
Lentzea terrae
Lentzea waywayandensis
Leptinotarsa decemlineata
Leptodesmis sichuanensis PKUAC-SCTA121
Leptodesmis sichuanensis
Leptolyngbya boryana IAM M-101
Leptolyngbya boryana IU 594
Leptolyngbya boryana NIES-2135
Leptolyngbya boryana dg5
Leptolyngbya boryana
Leptolyngbya ectocarpi
Leptolyngbya minuta LEGE 07181
Leptolyngbya ohadii
Leptolyngbya saxicola LEGE 07132
Leptolyngbya saxicola LEGE 07170
Leptolyngbya sp. ‘hensonii’
Leptolyngbya sp. 7M
Leptolyngbya sp. BC1307
Leptolyngbya sp. BL0902
Leptolyngbya sp. CCY15150
Leptolyngbya sp. CENA350
Leptolyngbya sp. CENA375
Leptolyngbya sp. Cla-17
Leptolyngbya sp. FACHB-1624
Leptolyngbya sp. FACHB-16
Leptolyngbya sp. FACHB-17
Leptolyngbya sp. FACHB-238
Leptolyngbya sp. FACHB-261
Leptolyngbya sp. FACHB-321
Leptolyngbya sp. FACHB-36
Leptolyngbya sp. FACHB-541
Leptolyngbya sp. FACHB-671
Leptolyngbya sp. FACHB-711
Leptolyngbya sp. FACHB-8
Leptolyngbya sp. GGD
Leptolyngbya sp. Heron Island J
Leptolyngbya sp. IPPAS B-1204
Leptolyngbya sp. KIOST-1
Leptolyngbya sp. LCH1
Leptolyngbya sp. LCM1.Bin17
Leptolyngbya sp. LCRNK_19
Leptolyngbya sp. LCRNK_20
Leptolyngbya sp. LCRNK_22
Leptolyngbya sp. LK
Leptolyngbya sp. MMG-1
Leptolyngbya sp. NIES-2104
Leptolyngbya sp. NIES-3755
Leptolyngbya sp. O-77
Leptolyngbya sp. PCC 6406
Leptolyngbya sp. PCC 7375
Leptolyngbya sp. PCC 7376
Leptolyngbya sp. PCC 7410
Leptolyngbya sp. PCC 8980
Leptolyngbya sp. PCC 9012
Leptolyngbya sp. PKUAC-SCTA174
Leptolyngbya sp. PKUAC-SCTB231
Leptolyngbya valderiana BDU 20041
Leptolyngbya
Leptolyngbyaceae cyanobacterium JSC-12
Leptomonas seymouri
Leptonema illini
Leptospira adleri
Leptospira ainazelensis
Leptospira alexanderi serovar Manhao 3 str. L 60
Leptospira alexanderi
Leptospira alstonii serovar Pingchang str. 80-412
Leptospira alstonii serovar Sichuanstr. 79601
Leptospira alstonii
Leptospira bandrabouensis
Leptospira biflexa serovar Patoc strain ‘Patoc 1 (Ames)’
Leptospira borgpetersenii serovar Pomona str. 200901868
Leptospira borgpetersenii str. Noumea 25
Leptospira borgpetersenii
Leptospira broomii serovar Hurstbridge str. 5399
Leptospira broomii
Leptospira ellinghausenii
Leptospira fainei serovar Hurstbridge str. BUT 6
Leptospira fainei
Leptospira haakeii
Leptospira idonii
Leptospira ilyithenensis
Leptospira inadai serovar Lyme str. 10
Leptospira inadai
Leptospira interrogans serovar Lora str. TE 1992
Leptospira interrogans
Leptospira kirschneri str. 2008720114
Leptospira kirschneri
Leptospira kobayashii
Leptospira koniambonensis
Leptospira licerasiae str. MMD4847
Leptospira meyeri serovar Hardjo str. Went 5
Leptospira meyeri serovar Semaranga str. Veldrot Semarang 173
Leptospira meyeri
Leptospira neocaledonica
Leptospira noguchii serovar Autumnalis str. ZUN142
Leptospira noguchii serovar Panama str. CZ214
Leptospira noguchii str. 1993005606
Leptospira noguchii str. 2001034031
Leptospira noguchii str. 2007001578
Leptospira noguchii
Leptospira ognonensis
Leptospira perolatii
Leptospira saintgironsiae
Leptospira sanjuanensis
Leptospira selangorensis
Leptospira sp. 1 VSF14
Leptospira sp. GIMC2001
Leptospira sp. ZV016
Leptospira terpstrae serovar Hualin str. LT 11-33 = ATCC 700639
Leptospira terpstrae
Leptospira tipperaryensis
Leptospira weilii serovar Ranarum str. ICFT
Leptospira weilii serovar Topaz str. LT2116
Leptospira weilii
Leptospira wolbachii serovar Codice str. CDC
Leptospira wolbachii
Leptospira yasudae
Leptospira
Leptospirillum ferriphilum YSK
Leptospirillum ferriphilum
Leptospirillum ferrodiazotrophum
Leptospirillum ferrooxidans C2-3
Leptospirillum ferrooxidans
Leptospirillum rubarum
Leptospirillum sp. Group II ‘5-way CG’
Leptospirillum sp. Group II ‘C75’
Leptospirillum sp. Group II ‘CF-1’
Leptospirillum sp. Group IV ‘UBA BS’
Leptospirillum
Leptothermofonsia sichuanensis PKUAC-SCTE412
Leptothermofonsia sichuanensis
Leptothoe kymatousa
Leptothoe spongobia
Leptothrix cholodnii SP-6
Leptothrix cholodnii
Leucobacter exalbidus
Leucobacter ruminantium
Leucobacter salsicius
Leucobacter sp. 7(1)
Levilactobacillus brevis
Lichenibacterium minor
Lichenibacterium ramalinae
Lichenibacterium sp. 6Y81
Lichenicoccus roseus
Limimaricola pyoseonensis
Limimaricola sp. ASW11-118
Limimonas halophila
Limisalsivibrio acetivorans
Limnobacter alexandrii
Limnobacter humi
Limnobacter sp. 130
Limnobacter sp. MED105
Limnobacter sp. SAORIC-580
Limnobacter sp. SAORIC-690
Limnochorda pilosa
Limnohabitans planktonicus II-D5
Limnohabitans planktonicus
Limnohabitans sp. 103DPR2
Limnohabitans sp. 15K
Limnohabitans sp. 2KL-17
Limnohabitans sp. 2KL-1
Limnohabitans sp. 2KL-27
Limnohabitans sp. 2KL-3
Limnohabitans sp. 2KL-51
Limnohabitans sp. 63ED37-2
Limnohabitans sp. B9-3
Limnohabitans sp. Bal53
Limnohabitans sp. DM1
Limnohabitans sp. G3-2
Limnohabitans sp. Hippo3
Limnohabitans sp. Jir72
Limnohabitans sp. MMS-10A-178
Limnohabitans sp. Rim11
Limnohabitans sp. Rim28
Limnohabitans sp. Rim47
Limnohabitans sp. WS1
Limnoraphis robusta CCAP 1446/4
Limnoraphis robusta CS-951
Limnoraphis robusta
Limnospira indica
Limnospira maxima CS-328
Limnospira
Limnothrix rosea
Limnothrix sp. FACHB-406
Limnothrix sp. FACHB-708
Limnothrix sp. FACHB-881
Limnothrix sp. PR1529
Limosa lapponica baueri
Limosilactobacillus fermentum 28-3-CHN
Limosilactobacillus fermentum NB-22
Limosilactobacillus fermentum
Limosilactobacillus reuteri subsp. rodentium
Limosilactobacillus reuteri
Lipotes vexillifer
Listeria innocua Clip11262
Listeria monocytogenes 08-5578
Listeria monocytogenes 08-5923
Listeria monocytogenes SLCC2372
Listeria monocytogenes SLCC2479
Listeria monocytogenes
Litoreibacter halocynthiae
Litoreibacter janthinus
Litoreibacter meonggei
Litoreibacter ponti
Litorilituus lipolyticus
Litorilituus sediminis
Litorimonas cladophorae
Litorivita pollutaquae
Loigolactobacillus bifermentans
Loigolactobacillus binensis
Loigolactobacillus coryniformis subsp. coryniformis KCTC 3167 = DSM 20001
Loigolactobacillus coryniformis subsp. coryniformis
Loigolactobacillus coryniformis subsp. torquens DSM 20004 = KCTC 3535
Loigolactobacillus coryniformis
Loigolactobacillus rennini
Loktanella atrilutea
Loktanella fryxellensis
Loktanella sp. DSM 29012
Loktanella sp. M215
Loktanella sp. PT4BL
Longibacter salinarum
Longitalea arenae
Longitalea luteola
Lonsdalea populi
Loripes lucinalis gill symbiont
Lucifera butyrica
Lujinxingia litoralis
Lujinxingia sediminis
Lujinxingia vulgaris
Lusitaniella coriacea LEGE 07167
Lusitaniella coriacea
Luteibacter anthropi
Luteibacter jiangsuensis
Luteibacter pinisoli
Luteibacter rhizovicinus DSM 16549
Luteibacter rhizovicinus
Luteibacter sp. 321
Luteibacter sp. 329MFSha
Luteibacter sp. 335
Luteibacter sp. 353
Luteibacter sp. EIF3
Luteibacter sp. OK325
Luteibacter sp. SG786
Luteibacter sp. Sphag1AF
Luteibacter sp. UNC138MFCol5.1
Luteibacter sp. UNCMF331Sha3.1
Luteibacter sp. UNCMF366Tsu5.1
Luteibacter sp. dw_328
Luteibacter yeojuensis
Luteimonas abyssi
Luteimonas aestuarii
Luteimonas arsenica
Luteimonas chenhongjianii
Luteimonas colneyensis
Luteimonas cucumeris
Luteimonas deserti
Luteimonas fraxinea
Luteimonas huabeiensis
Luteimonas padinae
Luteimonas saliphila
Luteimonas sp. 100069
Luteimonas sp. 9C
Luteimonas sp. BDR2-5
Luteimonas sp. FCS-9
Luteimonas sp. J16
Luteimonas sp. J29
Luteimonas sp. JM171
Luteimonas sp. MC1572
Luteimonas sp. MC1750
Luteimonas sp. MC1782
Luteimonas sp. MC1825
Luteimonas sp. MC1828
Luteimonas sp. MC1895
Luteimonas sp. MHLX1A
Luteimonas sp. RC10
Luteimonas sp. S4-F44
Luteimonas sp. XNQY3
Luteimonas sp. Y-2-2-4F
Luteimonas sp. YGD11-2
Luteimonas sp
Luteimonas terrae
Luteimonas viscosa
Luteimonas wenzhouensis
Luteimonas yindakuii
Luteipulveratus mongoliensis
Luteolibacter flavescens
Luteolibacter pohnpeiensis
Lutibacter agarilyticus
Lutibacter maritimus
Lutibacter oricola
Lutibacter sp. B1
Lutibacter sp. BRH_c52
Lutibacter sp. HS1-25
Lutimaribacter saemankumensis
Lyngbya aestuarii BL J
Lyngbya aestuarii PCC 7419
Lyngbya aestuarii
Lyngbya confervoides
Lyngbya lagerheimii UTEX 1930
Lyngbya sp. CCAP 1446/10
Lyngbya sp. PCC 8106
Lysinibacillus boronitolerans JCM 21713 = 10a = NBRC 103108
Lysinibacillus boronitolerans
Lysinibacillus contaminans
Lysinibacillus fusiformis ZB2
Lysinibacillus fusiformis ZC1
Lysinibacillus fusiformis
Lysinibacillus odysseyi 34hs-1 = NBRC 100172
Lysinibacillus odysseyi
Lysinibacillus sp. A1
Lysinibacillus sp. BF-4
Lysinibacillus sp. BK-16
Lysinibacillus sp. FJAT-14745
Lysinibacillus sp. LK3
Lysinibacillus sp. YS11
Lysinibacillus sp. ZYM-1
Lysinibacillus sphaericus OT4b.31
Lysinibacillus sphaericus
Lysinibacillus xylanilyticus
Lysinibacillus
Lysobacter aestuarii
Lysobacter alkalisoli
Lysobacter antarcticus
Lysobacter arseniciresistens ZS79
Lysobacter arseniciresistens
Lysobacter avium
Lysobacter bugurensis
Lysobacter capsici
Lysobacter ciconiae
Lysobacter defluvii IMMIB APB-9 = DSM 18482
Lysobacter defluvii
Lysobacter dokdonensis
Lysobacter enzymogenes
Lysobacter gilvus
Lysobacter luteus
Lysobacter oculi
Lysobacter pythonis
Lysobacter segetis
Lysobacter selenitireducens
Lysobacter soli
Lysobacter solisilvae
Lysobacter sp. A03
Lysobacter sp. A6
Lysobacter sp. CAU 1642
Lysobacter sp. GX 14042
Lysobacter sp. H21R4
Lysobacter sp. H23M47
Lysobacter sp. KIS68-7
Lysobacter sp. MMG2
Lysobacter sp. N42
Lysobacter sp. Root494
Lysobacter sp. Root690
Lysobacter sp. Root96
Lysobacter sp. TY2-98
Lysobacter sp. yr284
Lysobacter spongiae
Lysobacter spongiicola
Lysobacter tabacisoli
Lysobacter terrigena
Lysobacter tolerans
Lysobacter xinjiangensis
Lysobacter
Macaca mulatta
Macrococcus caseolyticus
Macrococcus hajekii
Mageeibacillus indolicus 0009-5
Mageeibacillus indolicus UPII9-5
Mageeibacillus indolicus
Magnetococcus marinus MC-1
Magnetococcus marinus
Magnetofaba australis IT-1
Magnetofaba australis
Magnetospira sp. QH-2
Magnetospirillum aberrantis
Magnetospirillum caucaseum
Magnetospirillum fulvum MGU-K5
Magnetospirillum fulvum
Magnetospirillum gryphiswaldense MSR-1 v2
Magnetospirillum gryphiswaldense MSR-1
Magnetospirillum gryphiswaldense
Magnetospirillum kuznetsovii
Magnetospirillum magneticum AMB-1
Magnetospirillum magneticum
Magnetospirillum magnetotacticum MS-1
Magnetospirillum magnetotacticum
Magnetospirillum marisnigri
Magnetospirillum molischianum DSM 120
Magnetospirillum molischianum
Magnetospirillum moscoviense
Magnetospirillum sp. 15-1
Magnetospirillum sp. J10
Magnetospirillum sp. LM-5
Magnetospirillum sp. ME-1
Magnetospirillum sp. SS-4
Magnetospirillum sp. UT-4
Magnetospirillum sp. XM-1
Magnetospirillum sp.
Magnetospirillum
Magnetovibrio blakemorei
Mahella australiensis 50-1 BON
Mahella australiensis
Malaciobacter canalis
Malaciobacter marinus
Malaciobacter molluscorum LMG 25693
Malaciobacter molluscorum
Malaciobacter pacificus
Maledivibacter halophilus
Malikia granosa
Malikia spinosa
Maliponia aquimaris
Malonomonas rubra DSM 5091
Malonomonas rubra
Mameliella alba
Mameliella sediminis
Mameliella sp. CS4
Mangrovactinospora gilvigrisea
Mangrovibacter phragmitis
Mangrovibacter plantisponsor
Mangrovibacter sp. MFB070
Mangrovibacter yixingensis
Mangrovibacter
Mangrovibacterium diazotrophicum
Mangrovibacterium lignilyticum
Mangrovibacterium marinum
Mangrovicoccus algicola
Mangrovicoccus sp. HB161399
Mangrovicoccus ximenensis
Mangrovimonas yunxiaonensis
Mangrovivirga sp. M17
Marasmitruncus massiliensis
Margalitia shackletonii
Maribacter antarcticus
Maribacter dokdonensis
Maribacter flavus
Maribacter hydrothermalis
Maribacter sp. PR66
Maribacter spongiicola
Maribacter
Maribellus comscasis
Maribellus sediminis
Maribellus
Maribius pelagius
Maribius salinus
Maricaulis alexandrii
Marichromatium bheemlicum
Marichromatium gracile
Marichromatium purpuratum 984
Marichromatium purpuratum
Marichromatium sp. AB31
Marichromatium sp. AB32
Marichromatium
Maridesulfovibrio bastinii
Maridesulfovibrio ferrireducens
Maridesulfovibrio frigidus
Maridesulfovibrio hydrothermalis AM13 = DSM 14728
Maridesulfovibrio hydrothermalis
Maridesulfovibrio salexigens DSM 2638
Maridesulfovibrio salexigens
Maridesulfovibrio zosterae
Marileptolyngbya sina SCSIO T-2
Marinactinospora thermotolerans DSM 45154
Marinactinospora thermotolerans
Marinagarivorans algicola
Marinibacterium profundimaris
Marinibactrum halimedae
Mariniblastus fucicola
Marinicaulis flavus
Marinigracilibium pacificum
Marinilabilia rubra
Marinilabilia salmonicolor
Marinilabiliales bacterium
Marinimicrobium agarilyticum
Marinimicrobium sp. LS-A18
Marinitoga hydrogenitolerans
Marinitoga lauensis
Marinitoga litoralis
Marinitoga sp. 1135
Marinitoga sp. 38H-ov
Marinitoga
Marinobacter adhaerens HP15
Marinobacter algicola DG893
Marinobacter algicola
Marinobacter caseinilyticus
Marinobacter daepoensis
Marinobacter daqiaonensis
Marinobacter excellens HL-55
Marinobacter excellens LAMA 842
Marinobacter halophilus
Marinobacter lipolyticus SM19
Marinobacter lipolyticus
Marinobacter manganoxydans MnI7-9
Marinobacter manganoxydans
Marinobacter psychrophilus
Marinobacter salarius
Marinobacter salicampi
Marinobacter santoriniensis NKSG1
Marinobacter santoriniensis
Marinobacter sp. AC-23
Marinobacter sp. Arc7-DN-1
Marinobacter sp. BSs20148
Marinobacter sp. DS40M8
Marinobacter sp. ELB17
Marinobacter sp. ES-1
Marinobacter sp. G11
Marinobacter sp. HL-58
Marinobacter sp. LV10MA510-1
Marinobacter sp. LV10R520-4
Marinobacter sp. P4B1
Marinobacter sp.
Marinobacter subterrani
Marinobacterium aestuarii
Marinobacterium arenosum
Marinobacterium jannaschii
Marinobacterium litorale
Marinobacterium lutimaris
Marinobacterium mangrovicola
Marinobacterium nitratireducens
Marinobacterium profundum
Marinobacterium ramblicola
Marinobacterium rhizophilum
Marinobacterium sedimentorum
Marinobacterium sp. AK27
Marinobacterium sp. xm-d-530
Marinobacterium sp. xm-d-564
Marinobacterium sp. xm-d-579
Marinobacterium zhoushanense
Marinococcus halophilus
Marinococcus halotolerans
Marinococcus luteus
Marinomonas algicola
Marinomonas mediterranea MMB-1
Marinomonas mediterranea
Marinomonas sp. KJ51-3
Marinospirillum alkaliphilum DSM 21637
Marinospirillum alkaliphilum
Marinospirillum perlucidum
Mariprofundus ferrooxydans PV-1
Mariprofundus ferrooxydans
Marisediminicola antarctica
Marisediminicola senii
Marisediminitalea aggregata
Maritalea mediterranea
Maritalea mobilis
Maritalea myrionectae
Maritimibacter harenae
Maritimibacter sp. 55A14
Marivirga atlantica
Marivirga sericea
Marivirga sp. S37H4
Marivita cryptomonadis
Marivita geojedonensis
Marivita sp. GX14005
Marivita sp. XM-24bin2
Marivita
Marmoricola aurantiacus
Marmoricola caldifontis
Marmoricola endophyticus
Marmoricola mangrovicus
Marmoricola scoriae
Marmoricola solisilvae
Marmoricola sp. Leaf446
Martelella alba
Martelella endophytica
Martelella mediterranea
Martelella sp. HB161492
Marvinbryantia formatexigens DSM 14469
Marvinbryantia formatexigens
Massilia agri
Massilia alkalitolerans
Massilia antarctica
Massilia antibiotica
Massilia aquatica
Massilia arenae
Massilia arenosa
Massilia aromaticivorans
Massilia atriviolacea
Massilia aurea
Massilia eurypsychrophila
Massilia forsythiae
Massilia genomosp. 1
Massilia glaciei
Massilia group
Massilia mucilaginosa
Massilia niastensis
Massilia norwichensis
Massilia oculi
Massilia phyllostachyos
Massilia pinisoli
Massilia putida
Massilia rhizosphaerae
Massilia rubra
Massilia soli
Massilia sp. 9096
Massilia sp. 9I
Massilia sp. BSC265
Massilia sp. Bi118
Massilia sp. CCM 8734
Massilia sp. DM-R-R2A-13
Massilia sp. Dwa41.01b
Massilia sp. H6
Massilia sp. HC52
Massilia sp. HP4
Massilia sp. IC2-278
Massilia sp. IC2-476
Massilia sp. IC2-477
Massilia sp. JS1662
Massilia sp. KIM
Massilia sp. LC238
Massilia sp. LPB0304
Massilia sp. Leaf139
Massilia sp. MAHUQ-52
Massilia sp. MS-15
Massilia sp. Mn16-1_5
Massilia sp. NEAU-DD11
Massilia sp. NR 4-1
Massilia sp. IC2-476
Massilia sp. PAMC28688
Massilia sp. PDC64
Massilia sp. Root1485
Massilia sp. Root335
Massilia sp. Root351
Massilia sp. Root418
Massilia sp. ST3
Massilia sp. TS11
Massilia sp. YMA4
Massilia terrae
Massilia timonae
Massilia violaceinigra
Massilia yuzhufengensis
Massilia
Massilibacillus massiliensis
Massilibacterium senegalense
Mastigocladopsis repens
Mastigocladus laminosus CCMEE 5186
Mastigocladus laminosus CCMEE 5192
Mastigocladus laminosus CCMEE 5193
Mastigocladus laminosus CCMEE 5198
Mastigocladus laminosus CCMEE 5201
Mastigocladus laminosus CCMEE 5202
Mastigocladus laminosus CCMEE 5203
Mastigocladus laminosus CCMEE 5204
Mastigocladus laminosus CCMEE 5205
Mastigocladus laminosus CCMEE 5207
Mastigocladus laminosus CCMEE 5208
Mastigocladus laminosus CCMEE 5267
Mastigocladus laminosus CCMEE 5268
Mastigocladus laminosus CCMEE 5272
Mastigocladus laminosus CCMEE 5273
Mastigocladus laminosus CCMEE 5281
Mastigocladus laminosus CCMEE 5282
Mastigocladus laminosus CCMEE 5318
Mastigocladus laminosus CCMEE 5319
Mastigocladus laminosus CCMEE 5320
Mastigocladus laminosus CCMEE 5321
Mastigocladus laminosus CCMEE 5323
Mastigocladus laminosus CCMEE 5324
Mastigocladus laminosus CCMEE 5326
Mastigocladus laminosus CCMEE 5327
Mastigocladus laminosus CCMEE 5328
Mastigocladus laminosus CCMEE 5329
Mastigocladus laminosus CCMEE 5331
Mastigocladus laminosus CCMEE 5332
Mastigocladus laminosus CCMEE 5335
Mastigocladus laminosus CCMEE 5337
Mastigocladus laminosus CCMEE 5379
Mastigocladus laminosus CCMEE 5380
Mastigocladus laminosus CG22
Mastigocladus laminosus Ind29
Mastigocladus laminosus NQAIF328
Mastigocladus laminosus UTEX LB 1931
Mastigocladus laminosus UU774
Mastigocladus laminosus W1110
Mastigocladus laminosus W112
Mastigocladus laminosus W114
Mastigocladus laminosus W118
Mastigocladus laminosus W119
Mastigocladus laminosus W156
Mastigocladus laminosus W157
Mastigocladus laminosus W213
Mastigocladus laminosus W226
Mastigocladus laminosus W241
Mastigocladus laminosus W246
Mastigocladus laminosus W247
Mastigocladus laminosus W249
Mastigocladus laminosus W3110
Mastigocladus laminosus W312
Mastigocladus laminosus W313
Mastigocladus laminosus W321
Mastigocladus laminosus W327
Mastigocladus laminosus W334
Mastigocladus laminosus W344
Mastigocladus laminosus W4110
Mastigocladus laminosus W411
Mastigocladus laminosus W412
Mastigocladus laminosus W422
Mastigocladus laminosus W435
Mastigocladus laminosus W4510
Mastigocladus laminosus W457
Mastigocladus laminosus W511
Mastigocladus laminosus W512
Mastigocladus laminosus W515
Mastigocladus laminosus W519
Mastigocladus laminosus W522
Mastigocladus laminosus W523
Mastigocladus laminosus
Mastigocladus sp. CHP1
Mastigocladus sp. CHP2
Mastigocladus sp. CHP3
Mastigocladus sp. FACHB-408
Mastigocladus sp. FACHB-785
Mastigocoleus testarum BC008
Mastigocoleus testarum
Medicago truncatula
Mediterraneibacter faecis
Mediterraneibacter sp. 210702-DFI.3.120
Mediterraneibacter sp. 210702-DFI.5.30
Mediterraneibacter
Megalodesulfovibrio gigas DSM 1382 = ATCC 19364
Megalodesulfovibrio gigas
Megasphaera butyrica
Megasphaera cerevisiae DSM 20462
Megasphaera cerevisiae
Megasphaera elsdenii 14-14
Megasphaera elsdenii CAG: 570
Megasphaera elsdenii DSM 20460
Megasphaera elsdenii
Megasphaera genomosp. type_1 str. 28L
Megasphaera genomosp. type_1
Megasphaera genomosp. type_2
Megasphaera hexanoica
Megasphaera hominis
Megasphaera hutchinsoni
Megasphaera lornae
Megasphaera massiliensis
Megasphaera micronuciformis
Megasphaera paucivorans
Megasphaera sp. AM44-1BH
Megasphaera sp. ASD88
Megasphaera sp. An286
Megasphaera sp. BL7
Megasphaera sp. DISK 18
Megasphaera sp. DJF_B143
Megasphaera sp. MJR8396C
Megasphaera sp. NM10
Megasphaera sp. SW808
Megasphaera sp. UPII 135-E
Megasphaera stantonii
Megasphaera vaginalis
Megasphaera
Meiothermus hypogaeus
Meiothermus luteus
Melaminivora alkalimesophila
Melaminivora suipulveris
Melittangium boletus DSM 14713
Meridianimarinicoccus roseus
Merismopedia glauca
Mesoaciditoga sp.
Mesobacillus boroniphilus JCM 21738
Mesobacillus campisalis
Mesobacillus persicus
Mesobacillus selenatarsenatis SF-1
Mesobacillus selenatarsenatis
Mesobacillus subterraneus
Mesobaculum littorinae
Mesonia oceanica
Mesonia sp. JHPTF-M18
Mesorhizobium abyssinicae
Mesorhizobium acaciae
Mesorhizobium albiziae
Mesorhizobium alhagi
Mesorhizobium amorphae CCNWGS0123
Mesorhizobium amorphae
Mesorhizobium atlanticum
Mesorhizobium australicum WSM2073
Mesorhizobium australicum
Mesorhizobium calcicola
Mesorhizobium camelthorni
Mesorhizobium cantuariense
Mesorhizobium caraganae
Mesorhizobium carmichaelinearum
Mesorhizobium chacoense
Mesorhizobium ciceri biovar biserrulae WSM1271
Mesorhizobium ciceri biovar biserrulae
Mesorhizobium ciceri
Mesorhizobium delmotii
Mesorhizobium erdmanii
Mesorhizobium escarrei
Mesorhizobium gobiense
Mesorhizobium hawassense
Mesorhizobium helmanticense
Mesorhizobium huakuii 7653R
Mesorhizobium huakuii
Mesorhizobium intechi
Mesorhizobium japonicum MAFF 303099
Mesorhizobium japonicum R7A
Mesorhizobium japonicum
Mesorhizobium jarvisii
Mesorhizobium kowhaii
Mesorhizobium loti NZP2037
Mesorhizobium loti R88b
Mesorhizobium loti
Mesorhizobium mediterraneum
Mesorhizobium metallidurans STM 2683
Mesorhizobium metallidurans
Mesorhizobium muleiense
Mesorhizobium neociceri
Mesorhizobium newzealandense
Mesorhizobium olivaresii
Mesorhizobium onobrychidis
Mesorhizobium opportunistum WSM2075
Mesorhizobium opportunistum
Mesorhizobium plurifarium
Mesorhizobium prunaredense
Mesorhizobium qingshengii
Mesorhizobium robiniae
Mesorhizobium sangaii
Mesorhizobium sanjuanii
Mesorhizobium septentrionale
Mesorhizobium shangrilense
Mesorhizobium silamurunense
Mesorhizobium sophorae
Mesorhizobium sp. 102-Evora
Mesorhizobium sp. 113-1-2
Mesorhizobium sp. 113-3-3
Mesorhizobium sp. 113-3-9
Mesorhizobium sp. 128a
Mesorhizobium sp. 131-2-1
Mesorhizobium sp. 131-2-5
Mesorhizobium sp. 131-3-5
Mesorhizobium sp. 64b-Beja
Mesorhizobium sp. 78-Elvas
Mesorhizobium sp. 85-Elvas
Mesorhizobium sp. 90-Evora
Mesorhizobium sp. 94-Evora
Mesorhizobium sp. 98-Evora
Mesorhizobium sp. 9H
Mesorhizobium sp. AA22
Mesorhizobium sp. AA23
Mesorhizobium sp. AC100c
Mesorhizobium sp. AC21c2
Mesorhizobium sp. AC39e1
Mesorhizobium sp. AC98e
Mesorhizobium sp. ACMP18
Mesorhizobium sp. AG15
Mesorhizobium sp. AG17
Mesorhizobium sp. AG1
Mesorhizobium sp. AG22
Mesorhizobium sp. AG27
Mesorhizobium sp. AG7
Mesorhizobium sp. AR02
Mesorhizobium sp. AR07
Mesorhizobium sp. AR10
Mesorhizobium sp. B2-1-3A
Mesorhizobium sp. B2-5-10
Mesorhizobium sp. B2-5-11
Mesorhizobium sp. B2-5-12
Mesorhizobium sp. B2-5-6
Mesorhizobium sp. B2-5-7
Mesorhizobium sp. B2-5-8
Mesorhizobium sp. B2-5-9
Mesorhizobium sp. B2-6-2
Mesorhizobium sp. B2-6-5
Mesorhizobium sp. B2-6-6
Mesorhizobium sp. B2-6-7
Mesorhizobium sp. B2-7-2
Mesorhizobium sp. B2-8-4
Mesorhizobium sp. B2-8-6
Mesorhizobium sp. B2-8-7
Mesorhizobium sp. B2-8-8
Mesorhizobium sp. B2-8-9
Mesorhizobium sp. BA134
Mesorhizobium sp. BA151
Mesorhizobium sp. BD56
Mesorhizobium sp. BIHB 4151
Mesorhizobium sp. BIHB 4154
Mesorhizobium sp. BIHB 4158
Mesorhizobium sp. BR-8-Braganca
Mesorhizobium sp. BR3804
Mesorhizobium sp. C-25-Coimbra
Mesorhizobium sp. C-3-Coimbra
Mesorhizobium sp. C-9-Coimbra
Mesorhizobium sp. CCANP117
Mesorhizobium sp. CCANP11
Mesorhizobium sp. CCANP130
Mesorhizobium sp. CCANP133
Mesorhizobium sp. CCANP29
Mesorhizobium sp. CCANP33
Mesorhizobium sp. CCANP55
Mesorhizobium sp. CCANP58
Mesorhizobium sp. CCANP61
Mesorhizobium sp. CCANP63
Mesorhizobium sp. CCANP68
Mesorhizobium sp. CCANP84
Mesorhizobium sp. CCANP87
Mesorhizobium sp. CCANP96
Mesorhizobium sp. CCBAU 01557
Mesorhizobium sp. CCBAU 01577
Mesorhizobium sp. CCBAU 01597
Mesorhizobium sp. CCBAU 01602
Mesorhizobium sp. CCBAU 01606
Mesorhizobium sp. CCBAU 03399
Mesorhizobium sp. CCBAU 03405
Mesorhizobium sp. CCBAU 03418
Mesorhizobium sp. CCBAU 03423
Mesorhizobium sp. CCBAU 03427
Mesorhizobium sp. CCBAU 03430
Mesorhizobium sp. CCBAU 03483
Mesorhizobium sp. CCBAU 03485
Mesorhizobium sp. CCBAU 03505
Mesorhizobium sp. CCBAU 03524
Mesorhizobium sp. CCBAU 03535
Mesorhizobium sp. CCBAU 03599
Mesorhizobium sp. CCBAU 03605
Mesorhizobium sp. CCBAU 03611
Mesorhizobium sp. CCBAU 11166
Mesorhizobium sp. CCBAU 11185
Mesorhizobium sp. CCBAU 11196
Mesorhizobium sp. CCBAU 11206
Mesorhizobium sp. CCBAU 11214
Mesorhizobium sp. CCBAU 11217
Mesorhizobium sp. CCBAU 11226
Mesorhizobium sp. CCBAU 11231
Mesorhizobium sp. CCBAU 11257
Mesorhizobium sp. CCBAU 11270
Mesorhizobium sp. CCBAU 11300
Mesorhizobium sp. CCBAU 11510
Mesorhizobium sp. CCBAU 11523
Mesorhizobium sp. CCBAU 11528
Mesorhizobium sp. CCBAU 23335
Mesorhizobium sp. CCBAU 23359
Mesorhizobium sp. CCBAU 23361
Mesorhizobium sp. CCBAU 23368
Mesorhizobium sp. CCBAU 25282
Mesorhizobium sp. CCBAU 33359
Mesorhizobium sp. CCBAU 33366
Mesorhizobium sp. CCBAU 33385
Mesorhizobium sp. CCBAU 33395
Mesorhizobium sp. CCBAU 33404
Mesorhizobium sp. CCBAU 33406
Mesorhizobium sp. CCBAU 33423
Mesorhizobium sp. CCBAU 33436
Mesorhizobium sp. CCBAU 33443
Mesorhizobium sp. CCBAU 33451
Mesorhizobium sp. CCBAU 33454
Mesorhizobium sp. CCBAU 33464
Mesorhizobium sp. CCBAU 33470
Mesorhizobium sp. CCBAU 33475
Mesorhizobium sp. CCBAU 33477
Mesorhizobium sp. CCBAU 35331
Mesorhizobium sp. CCBAU 35339
Mesorhizobium sp. CCBAU 35340
Mesorhizobium sp. CCBAU 35343
Mesorhizobium sp. CCBAU 35350
Mesorhizobium sp. CCBAU 35356
Mesorhizobium sp. CCBAU 35357
Mesorhizobium sp. CCBAU 41335
Mesorhizobium sp. CCBAU 41350
Mesorhizobium sp. CCBAU 41384
Mesorhizobium sp. CCBAU 41403
Mesorhizobium sp. CCBAU 71303
Mesorhizobium sp. CCBAU 71320
Mesorhizobium sp. CCBAU 71358
Mesorhizobium sp. CCBAU 73184
Mesorhizobium sp. CCBAU 73199
Mesorhizobium sp. CCBAU 73204
Mesorhizobium sp. CCBAU 73223
Mesorhizobium sp. CCBAU 73245
Mesorhizobium sp. CCBAU 73248
Mesorhizobium sp. CCBAU 73255
Mesorhizobium sp. CCBAU 73277
Mesorhizobium sp. CCBAU 75057
Mesorhizobium sp. CCBAU 75066
Mesorhizobium sp. CCBAU 75084
Mesorhizobium sp. CCBAU 75093
Mesorhizobium sp. CCBAU 75095
Mesorhizobium sp. CCBAU 75122
Mesorhizobium sp. CCBAU 75128
Mesorhizobium sp. CCBAU 75133
Mesorhizobium sp. CCBAU 75140
Mesorhizobium sp. CCBAU 75176
Mesorhizobium sp. CCBAU 75179
Mesorhizobium sp. CCBAU 75206
Mesorhizobium sp. CCBAU 75220
Mesorhizobium sp. CCBAU 75225
Mesorhizobium sp. CCBAU 75263
Mesorhizobium sp. CCBAU 831015
Mesorhizobium sp. CCBAU 83919
Mesorhizobium sp. CCBAU 83923
Mesorhizobium sp. CCBAU 83935
Mesorhizobium sp. CCBAU 83943
Mesorhizobium sp. CCBAU 83948
Mesorhizobium sp. CCBAU 83951
Mesorhizobium sp. CCBAU 83953
Mesorhizobium sp. CCBAU 83978
Mesorhizobium sp. CCBAU 83979
Mesorhizobium sp. CCBAU 83980
Mesorhizobium sp. CCBAU 83994
Mesorhizobium sp. CCNWGS0010-1
Mesorhizobium sp. CCNWGS0011
Mesorhizobium sp. CCNWGS0015-1
Mesorhizobium sp. CCNWSX622
Mesorhizobium sp. CCNWSX661
Mesorhizobium sp. CCNWSX691
Mesorhizobium sp. CIAM0210
Mesorhizobium sp. CMSS27
Mesorhizobium sp. CR-32-Caldas da Rainha
Mesorhizobium sp. CS13166
Mesorhizobium sp. CV-18-Elvas
Mesorhizobium sp. D11b
Mesorhizobium sp. D15
Mesorhizobium sp. D17
Mesorhizobium sp. D21b
Mesorhizobium sp. D22a
Mesorhizobium sp. D23
Mesorhizobium sp. D25a
Mesorhizobium sp. D26a
Mesorhizobium sp. D27
Mesorhizobium sp. D34
Mesorhizobium sp. D35
Mesorhizobium sp. DUS466
Mesorhizobium sp. DWO366
Mesorhizobium sp. G-10-Guarda
Mesorhizobium sp. G-55-Guarda
Mesorhizobium sp. GGS20
Mesorhizobium sp. GMSS1
Mesorhizobium sp. GMSS22
Mesorhizobium sp. HAMBI 1487
Mesorhizobium sp. HAMBI 260
Mesorhizobium sp. ICMP 11539
Mesorhizobium sp. ICMP 11541
Mesorhizobium sp. ICMP 11708
Mesorhizobium sp. ICMP 11719
Mesorhizobium sp. ICMP 11720
Mesorhizobium sp. ICMP 11721
Mesorhizobium sp. ICMP 11722
Mesorhizobium sp. ICMP 11726
Mesorhizobium sp. ICMP 11736
Mesorhizobium sp. ICMP 12635
Mesorhizobium sp. ICMP 12638
Mesorhizobium sp. ICMP 12649
Mesorhizobium sp. ICMP 13190
Mesorhizobium sp. ICMP 13193
Mesorhizobium sp. ICMP 18943
Mesorhizobium sp. ICMP 19041
Mesorhizobium sp. ICMP 19043
Mesorhizobium sp. ICMP 19418
Mesorhizobium sp. ICMP 19419
Mesorhizobium sp. ICMP 19513
Mesorhizobium sp. ICMP 19550
Mesorhizobium sp. ICMP 19570
Mesorhizobium sp. ICMP 19572
Mesorhizobium sp. ICMP 19850
Mesorhizobium sp. ICMP 19851
Mesorhizobium sp. ICMP 19852
Mesorhizobium sp. INPA78B
Mesorhizobium sp. ISA0605
Mesorhizobium sp. J8
Mesorhizobium sp. K13a
Mesorhizobium sp. K18
Mesorhizobium sp. K21
Mesorhizobium sp. K26
Mesorhizobium sp. K27a
Mesorhizobium sp. K27b
Mesorhizobium sp. K28a
Mesorhizobium sp. K28b
Mesorhizobium sp. K36
Mesorhizobium sp. L-2-11
Mesorhizobium sp. L-8-10
Mesorhizobium sp. L-8-3
Mesorhizobium sp. L103C105A0
Mesorhizobium sp. L103C119B0
Mesorhizobium sp. L103C120A0
Mesorhizobium sp. L103C131B0
Mesorhizobium sp. L2C054A000
Mesorhizobium sp. L2C066B000
Mesorhizobium sp. L2C067A000
Mesorhizobium sp. L2C084A000
Mesorhizobium sp. L2C085B000
Mesorhizobium sp. L2C089B000
Mesorhizobium sp. L48C026A00
Mesorhizobium sp. LCM 4576
Mesorhizobium sp. LLAN46
Mesorhizobium sp. LLAN64
Mesorhizobium sp. LLAN66
Mesorhizobium sp. LLAN67
Mesorhizobium sp. LM-18-Lamego
Mesorhizobium sp. LM-9-Lamego
Mesorhizobium sp. LMG 4270
Mesorhizobium sp. LMS-1
Mesorhizobium sp. LNHC209A00
Mesorhizobium sp. LNHC220B00
Mesorhizobium sp. LNHC221B00
Mesorhizobium sp. LNHC229A00
Mesorhizobium sp. LNHC232B00
Mesorhizobium sp. LNHC252B00
Mesorhizobium sp. LNJC372A00
Mesorhizobium sp. LNJC374B00
Mesorhizobium sp. LNJC384A00
Mesorhizobium sp. LNJC391B00
Mesorhizobium sp. LNJC394B00
Mesorhizobium sp. LNJC395A00
Mesorhizobium sp. LNJC399B00
Mesorhizobium sp. LNJC405B00
Mesorhizobium sp. LSHC412B00
Mesorhizobium sp. LSHC414A00
Mesorhizobium sp. LSHC416B00
Mesorhizobium sp. LSHC420B00
Mesorhizobium sp. LSHC422A00
Mesorhizobium sp. LSHC424B00
Mesorhizobium sp. LSHC426A00
Mesorhizobium sp. LSHC440A00
Mesorhizobium sp. LSHC440B00
Mesorhizobium sp. LSJC255A00
Mesorhizobium sp. LSJC264A00
Mesorhizobium sp. LSJC265A00
Mesorhizobium sp. LSJC268A00
Mesorhizobium sp. LSJC269B00
Mesorhizobium sp. LSJC277A00
Mesorhizobium sp. LSJC280B00
Mesorhizobium sp. LSJC285A00
Mesorhizobium sp. Lal1
Mesorhizobium sp. Lal2
Mesorhizobium sp. Lo5-9
Mesorhizobium sp. Lpe11
Mesorhizobium sp. Lpe12
Mesorhizobium sp. Lpe15
Mesorhizobium sp. LuLit12
Mesorhizobium sp. M00.F.Ca.ET.038.03.1.1
Mesorhizobium sp. M00.F.Ca.ET.149.01.1.1
Mesorhizobium sp. M00.F.Ca.ET.151.01.1.1
Mesorhizobium sp. M00.F.Ca.ET.158.01.1.1
Mesorhizobium sp. M00.F.Ca.ET.170.01.1.1
Mesorhizobium sp. M00.F.Ca.ET.186.01.1.1
Mesorhizobium sp. M00.F.Ca.ET.216.01.1.1
Mesorhizobium sp. M00.F.Ca.ET.217.01.1.1
Mesorhizobium sp. M00.F.Ca.ET.220.01.1.1
Mesorhizobium sp. M1A.F.Ca.ET.072.01.1.1
Mesorhizobium sp. M1A.F.Ca.IN.020.03.1.1
Mesorhizobium sp. M1A.F.Ca.IN.020.03.2.1
Mesorhizobium sp. M1A.F.Ca.IN.020.04.1.1
Mesorhizobium sp. M1A.F.Ca.IN.020.06.1.1
Mesorhizobium sp. M1A.F.Ca.IN.020.30.1.1
Mesorhizobium sp. M1A.F.Ca.IN.020.32.1.1
Mesorhizobium sp. M1A.F.Ca.IN.022.02.1.1
Mesorhizobium sp. M1A.F.Ca.IN.022.04.1.1
Mesorhizobium sp. M1A.F.Ca.IN.022.05.2.1
Mesorhizobium sp. M1A.F.Ca.IN.022.06.1.1
Mesorhizobium sp. M1A.F.Ca.IN.022.07.1.1
Mesorhizobium sp. M1A.T.Ca.IN.004.03.1.1
Mesorhizobium sp. M1B.F.Ca.ET.045.04.1.1
Mesorhizobium sp. M1C.F.Ca.ET.144.01.1.1
Mesorhizobium sp. M1C.F.Ca.ET.176.01.1.1
Mesorhizobium sp. M1C.F.Ca.ET.187.01.1.1
Mesorhizobium sp. M1C.F.Ca.ET.188.01.1.1
Mesorhizobium sp. M1C.F.Ca.ET.189.01.1.1
Mesorhizobium sp. M1C.F.Ca.ET.192.01.1.1
Mesorhizobium sp. M1C.F.Ca.ET.193.01.1.1
Mesorhizobium sp. M1C.F.Ca.ET.195.01.1.1
Mesorhizobium sp. M1C.F.Ca.ET.196.01.1.1
Mesorhizobium sp. M1C.F.Ca.ET.204.01.1.1
Mesorhizobium sp. M1C.F.Ca.ET.210.01.1.1
Mesorhizobium sp. M1C.F.Ca.ET.212.01.1.1
Mesorhizobium sp. M1D.F.Ca.ET.043.01.1.1
Mesorhizobium sp. M1D.F.Ca.ET.183.01.1.1
Mesorhizobium sp. M1D.F.Ca.ET.184.01.1.1
Mesorhizobium sp. M1D.F.Ca.ET.231.01.1.1
Mesorhizobium sp. M1D.F.Ca.ET.234.01.1.1
Mesorhizobium sp. M1E.F.Ca.ET.041.01.1.1
Mesorhizobium sp. M1E.F.Ca.ET.045.02.1.1
Mesorhizobium sp. M1E.F.Ca.ET.063.01.1.1
Mesorhizobium sp. M2A.F.Ca.ET.015.02.1.1
Mesorhizobium sp. M2A.F.Ca.ET.017.03.2.1
Mesorhizobium sp. M2A.F.Ca.ET.029.05.1.1
Mesorhizobium sp. M2A.F.Ca.ET.037.01.1.1
Mesorhizobium sp. M2A.F.Ca.ET.039.01.1.1
Mesorhizobium sp. M2A.F.Ca.ET.040.01.1.1
Mesorhizobium sp. M2A.F.Ca.ET.042.01.1.1
Mesorhizobium sp. M2A.F.Ca.ET.043.02.1.1
Mesorhizobium sp. M2A.F.Ca.ET.043.05.1.1
Mesorhizobium sp. M2A.F.Ca.ET.046.02.1.1
Mesorhizobium sp. M2A.F.Ca.ET.046.03.2.1
Mesorhizobium sp. M2A.F.Ca.ET.067.02.1.1
Mesorhizobium sp. M2D.F.Ca.ET.140.01.1.1
Mesorhizobium sp. M2D.F.Ca.ET.145.01.1.1
Mesorhizobium sp. M2D.F.Ca.ET.147.01.1.1
Mesorhizobium sp. M2D.F.Ca.ET.148.01.1.1
Mesorhizobium sp. M2D.F.Ca.ET.153.01.1.1
Mesorhizobium sp. M2D.F.Ca.ET.160.01.1.1
Mesorhizobium sp. M2D.F.Ca.ET.171.01.1.1
Mesorhizobium sp. M2D.F.Ca.ET.178.01.1.1
Mesorhizobium sp. M2D.F.Ca.ET.185.01.1.1
Mesorhizobium sp. M2D.F.Ca.ET.223.01.1.1
Mesorhizobium sp. M2D.F.Ca.ET.224.01.1.1
Mesorhizobium sp. M2D.F.Ca.ET.225.01.1.1
Mesorhizobium sp. M2D.F.Ca.ET.226.01.1.1
Mesorhizobium sp. M2D.F.Ca.ET.232.01.1.1
Mesorhizobium sp. M2D.F.Ca.ET.233.01.1.1
Mesorhizobium sp. M2E.F.Ca.ET.154.01.1.1
Mesorhizobium sp. M2E.F.Ca.ET.166.01.1.1
Mesorhizobium sp. M2E.F.Ca.ET.209.01.1.1
Mesorhizobium sp. M2E.F.Ca.ET.219.01.1.1
Mesorhizobium sp. M3A.F.Ca.ET.080.04.2.1
Mesorhizobium sp. M3A.F.Ca.ET.174.01.1.1
Mesorhizobium sp. M3A.F.Ca.ET.175.01.1.1
Mesorhizobium sp. M3A.F.Ca.ET.201.01.1.1
Mesorhizobium sp. M4A.F.Ca.ET.020.02.1.1
Mesorhizobium sp. M4A.F.Ca.ET.022.05.2.1
Mesorhizobium sp. M4A.F.Ca.ET.029.04.2.1
Mesorhizobium sp. M4A.F.Ca.ET.050.02.1.1
Mesorhizobium sp. M4A.F.Ca.ET.090.04.2.1
Mesorhizobium sp. M4B.F.Ca.ET.013.02.1.1
Mesorhizobium sp. M4B.F.Ca.ET.017.02.2.1
Mesorhizobium sp. M4B.F.Ca.ET.019.03.1.1
Mesorhizobium sp. M4B.F.Ca.ET.049.02.1.2
Mesorhizobium sp. M4B.F.Ca.ET.058.02.1.1
Mesorhizobium sp. M4B.F.Ca.ET.088.02.2.1
Mesorhizobium sp. M4B.F.Ca.ET.089.01.1.1
Mesorhizobium sp. M4B.F.Ca.ET.143.01.1.1
Mesorhizobium sp. M4B.F.Ca.ET.150.01.1.1
Mesorhizobium sp. M4B.F.Ca.ET.169.01.1.1
Mesorhizobium sp. M4B.F.Ca.ET.172.01.1.1
Mesorhizobium sp. M4B.F.Ca.ET.190.01.1.1
Mesorhizobium sp. M4B.F.Ca.ET.200.01.1.1
Mesorhizobium sp. M4B.F.Ca.ET.203.01.1.1
Mesorhizobium sp. M4B.F.Ca.ET.211.01.1.1
Mesorhizobium sp. M4B.F.Ca.ET.214.01.1.1
Mesorhizobium sp. M4B.F.Ca.ET.215.01.1.1
Mesorhizobium sp. M5C.F.Ca.ET.164.01.1.1
Mesorhizobium sp. M5C.F.Ca.IN.020.14.1.1
Mesorhizobium sp. M5C.F.Ca.IN.020.29.1.1
Mesorhizobium sp. M5C.F.Ca.IN.020.32.2.1
Mesorhizobium sp. M5C.F.Cr.IN.023.01.1.1
Mesorhizobium sp. M6A.T.Ca.TU.002.02.2.1
Mesorhizobium sp. M6A.T.Ce.TU.002.03.1.1
Mesorhizobium sp. M6A.T.Ce.TU.016.01.1.1
Mesorhizobium sp. M6A.T.Cr.TU.014.01.1.1
Mesorhizobium sp. M6A.T.Cr.TU.016.01.1.1
Mesorhizobium sp. M6A.T.Cr.TU.017.01.1.1
Mesorhizobium sp. M72P
Mesorhizobium sp. M7A.F.Ca.AU.001.01.1.1
Mesorhizobium sp. M7A.F.Ca.AU.002.02.1.1
Mesorhizobium sp. M7A.F.Ca.AU.002.03.1.1
Mesorhizobium sp. M7A.F.Ca.AU.002.04.1.1
Mesorhizobium sp. M7A.F.Ca.AU.002.06.1.1
Mesorhizobium sp. M7A.F.Ca.CA.001.04.1.1
Mesorhizobium sp. M7A.F.Ca.CA.001.04.2.1
Mesorhizobium sp. M7A.F.Ca.CA.001.05.1.1
Mesorhizobium sp. M7A.F.Ca.CA.001.06.1.1
Mesorhizobium sp. M7A.F.Ca.CA.001.07.2.1
Mesorhizobium sp. M7A.F.Ca.CA.001.08.1.1
Mesorhizobium sp. M7A.F.Ca.CA.001.08.2.1
Mesorhizobium sp. M7A.F.Ca.CA.001.09.1.1
Mesorhizobium sp. M7A.F.Ca.CA.001.09.2.1
Mesorhizobium sp. M7A.F.Ca.CA.001.10.2.1
Mesorhizobium sp. M7A.F.Ca.CA.001.11.2.1
Mesorhizobium sp. M7A.F.Ca.CA.001.12.1.1
Mesorhizobium sp. M7A.F.Ca.CA.001.12.2.1
Mesorhizobium sp. M7A.F.Ca.CA.001.13.1.1
Mesorhizobium sp. M7A.F.Ca.CA.001.13.2.1
Mesorhizobium sp. M7A.F.Ca.CA.001.14.1.1
Mesorhizobium sp. M7A.F.Ca.CA.001.15.1.1
Mesorhizobium sp. M7A.F.Ca.CA.001.16.1.1
Mesorhizobium sp. M7A.F.Ca.CA.002.03.1.1
Mesorhizobium sp. M7A.F.Ca.CA.002.03.2.1
Mesorhizobium sp. M7A.F.Ca.CA.002.04.1.1
Mesorhizobium sp. M7A.F.Ca.CA.002.05.1.1
Mesorhizobium sp. M7A.F.Ca.CA.002.06.1.1
Mesorhizobium sp. M7A.F.Ca.CA.002.07.1.1
Mesorhizobium sp. M7A.F.Ca.CA.002.09.1.1
Mesorhizobium sp. M7A.F.Ca.CA.002.10.1.1
Mesorhizobium sp. M7A.F.Ca.CA.002.11.2.1
Mesorhizobium sp. M7A.F.Ca.CA.002.12.1.1
Mesorhizobium sp. M7A.F.Ca.CA.002.14.1.2
Mesorhizobium sp. M7A.F.Ca.CA.002.15.1.1
Mesorhizobium sp. M7A.F.Ca.CA.002.15.2.1
Mesorhizobium sp. M7A.F.Ca.CA.003.01.2.1
Mesorhizobium sp. M7A.F.Ca.CA.004.01.1.1
Mesorhizobium sp. M7A.F.Ca.CA.004.02.1.1
Mesorhizobium sp. M7A.F.Ca.CA.004.04.1.1
Mesorhizobium sp. M7A.F.Ca.CA.004.04.2.1
Mesorhizobium sp. M7A.F.Ca.CA.004.05.1.1
Mesorhizobium sp. M7A.F.Ca.CA.004.05.2.1
Mesorhizobium sp. M7A.F.Ca.CA.004.06.1.1
Mesorhizobium sp. M7A.F.Ca.CA.004.06.2.1
Mesorhizobium sp. M7A.F.Ca.CA.004.07.1.1
Mesorhizobium sp. M7A.F.Ca.CA.004.08.1.1
Mesorhizobium sp. M7A.F.Ca.CA.004.08.2.1
Mesorhizobium sp. M7A.F.Ca.CA.004.09.1.2
Mesorhizobium sp. M7A.F.Ca.CA.004.10.1.1
Mesorhizobium sp. M7A.F.Ca.CA.004.11.1.1
Mesorhizobium sp. M7A.F.Ca.CA.004.11.2.1
Mesorhizobium sp. M7A.F.Ca.CA.004.12.1.1
Mesorhizobium sp. M7A.F.Ca.ET.027.02.1.1
Mesorhizobium sp. M7A.F.Ca.ET.027.03.2.1
Mesorhizobium sp. M7A.F.Ca.MR.148.00.0.0
Mesorhizobium sp. M7A.F.Ca.MR.176.00.0.0
Mesorhizobium sp. M7A.F.Ca.MR.228.00.0.0
Mesorhizobium sp. M7A.F.Ca.MR.245.00.0.0
Mesorhizobium sp. M7A.F.Ca.MR.362.00.0.0
Mesorhizobium sp. M7A.F.Ca.US.001.01.1.1
Mesorhizobium sp. M7A.F.Ca.US.001.02.1.1
Mesorhizobium sp. M7A.F.Ca.US.001.04.1.1
Mesorhizobium sp. M7A.F.Ca.US.001.04.2.1
Mesorhizobium sp. M7A.F.Ca.US.002.01.1.1
Mesorhizobium sp. M7A.F.Ca.US.003.02.1.1
Mesorhizobium sp. M7A.F.Ca.US.003.02.2.1
Mesorhizobium sp. M7A.F.Ca.US.005.03.1.1
Mesorhizobium sp. M7A.F.Ca.US.005.03.2.1
Mesorhizobium sp. M7A.F.Ca.US.006.01.1.1
Mesorhizobium sp. M7A.F.Ca.US.006.01.2.1
Mesorhizobium sp. M7A.F.Ca.US.006.04.2.1
Mesorhizobium sp. M7A.F.Ca.US.007.01.1.1
Mesorhizobium sp. M7A.F.Ca.US.007.01.2.1
Mesorhizobium sp. M7A.F.Ca.US.008.03.1.1
Mesorhizobium sp. M7A.F.Ca.US.010.02.1.1
Mesorhizobium sp. M7A.F.Ca.US.011.01.1.1
Mesorhizobium sp. M7A.F.Ca.US.014.04.1.1
Mesorhizobium sp. M7A.F.Ce.TU.012.03.2.1
Mesorhizobium sp. M7A.T.Ca.TU.009.01.1.1
Mesorhizobium sp. M7A.T.Ca.TU.009.01.1.2
Mesorhizobium sp. M7A.T.Ca.TU.009.01.3.1
Mesorhizobium sp. M7A.T.Ca.TU.009.01.3.2
Mesorhizobium sp. M7A.T.Ca.TU.009.02.1.1
Mesorhizobium sp. M7A.T.Ca.US.000.02.1.1
Mesorhizobium sp. M7A.T.Ca.US.000.02.2.1
Mesorhizobium sp. M7D.F.Ca.US.004.01.2.1
Mesorhizobium sp. M7D.F.Ca.US.004.03.1.1
Mesorhizobium sp. M7D.F.Ca.US.005.01.1.1
Mesorhizobium sp. M8A.F.Ca.ET.021.01.1.1
Mesorhizobium sp. M8A.F.Ca.ET.023.01.1.1
Mesorhizobium sp. M8A.F.Ca.ET.023.02.2.1
Mesorhizobium sp. M8A.F.Ca.ET.057.01.1.1
Mesorhizobium sp. M8A.F.Ca.ET.059.01.1.1
Mesorhizobium sp. M8A.F.Ca.ET.142.01.1.1
Mesorhizobium sp. M8A.F.Ca.ET.161.01.1.1
Mesorhizobium sp. M8A.F.Ca.ET.165.01.1.1
Mesorhizobium sp. M8A.F.Ca.ET.167.01.1.1
Mesorhizobium sp. M8A.F.Ca.ET.173.01.1.1
Mesorhizobium sp. M8A.F.Ca.ET.181.01.1.1
Mesorhizobium sp. M8A.F.Ca.ET.182.01.1.1
Mesorhizobium sp. M8A.F.Ca.ET.197.01.1.1
Mesorhizobium sp. M8A.F.Ca.ET.198.01.1.1
Mesorhizobium sp. M8A.F.Ca.ET.202.01.1.1
Mesorhizobium sp. M8A.F.Ca.ET.207.01.1.1
Mesorhizobium sp. M8A.F.Ca.ET.208.01.1.1
Mesorhizobium sp. M8A.F.Ca.ET.213.01.1.1
Mesorhizobium sp. M8A.F.Ca.ET.218.01.1.1
Mesorhizobium sp. M9A.F.Ca.ET.002.03.1.2
Mesorhizobium sp. ML105
Mesorhizobium sp. ML108
Mesorhizobium sp. ML128
Mesorhizobium sp. MM5333
Mesorhizobium sp. MM5334
Mesorhizobium sp. MM5336
Mesorhizobium sp. MM5343
Mesorhizobium sp. MM5352
Mesorhizobium sp. MM5357
Mesorhizobium sp. MM5360
Mesorhizobium sp. MM5361
Mesorhizobium sp. MM5364
Mesorhizobium sp. MM5369
Mesorhizobium sp. MM5370
Mesorhizobium sp. MM5372
Mesorhizobium sp. MM5376
Mesorhizobium sp. MM5377
Mesorhizobium sp. MM5382
Mesorhizobium sp. MM5397
Mesorhizobium sp. MM5440
Mesorhizobium sp. MM5462
Mesorhizobium sp. MM5618
Mesorhizobium sp. MM5675
Mesorhizobium sp. MM5734
Mesorhizobium sp. MM5757
Mesorhizobium sp. N105
Mesorhizobium sp. N141
Mesorhizobium sp. N2076
Mesorhizobium sp. N33
Mesorhizobium sp. N362
Mesorhizobium sp. N3
Mesorhizobium sp. N441
Mesorhizobium sp. NFY11
Mesorhizobium sp. NFY12
Mesorhizobium sp. NFY13
Mesorhizobium sp. NFY6
Mesorhizobium sp. NPY4
Mesorhizobium sp. NRY10
Mesorhizobium sp. NTY3
Mesorhizobium sp. NTY5
Mesorhizobium sp. NTY7
Mesorhizobium sp. NTY9
Mesorhizobium sp. NWGS01
Mesorhizobium sp. NWGS03
Mesorhizobium sp. NWGS05
Mesorhizobium sp. NWGS09
Mesorhizobium sp. NWNX05
Mesorhizobium sp. NWSX06
Mesorhizobium sp. NWSX19
Mesorhizobium sp. NWSX20
Mesorhizobium sp. NWSX22
Mesorhizobium sp. NWSX23
Mesorhizobium sp. NWSX24
Mesorhizobium sp. NWSX25
Mesorhizobium sp. NWXJ08
Mesorhizobium sp. NWXJ18
Mesorhizobium sp. NWXJ21
Mesorhizobium sp. NWXJ25
Mesorhizobium sp. NWXJ27
Mesorhizobium sp. NWXJ28
Mesorhizobium sp. NWXJ30
Mesorhizobium sp. NWXJ31
Mesorhizobium sp. NWXJ32
Mesorhizobium sp. NWXJ33
Mesorhizobium sp. NWXJ36
Mesorhizobium sp. NWXJ39
Mesorhizobium sp. NWXJ40
Mesorhizobium sp. NWXJ42
Mesorhizobium sp. NWXJ43
Mesorhizobium sp. NZP2077
Mesorhizobium sp. NZP2234
Mesorhizobium sp. NZP2298
Mesorhizobium sp. OD108
Mesorhizobium sp. OD119
Mesorhizobium sp. OD13
Mesorhizobium sp. OD14
Mesorhizobium sp. OD15
Mesorhizobium sp. OD18
Mesorhizobium sp. OD26
Mesorhizobium sp. OD31
Mesorhizobium sp. OD32
Mesorhizobium sp. OD42
Mesorhizobium sp. OD47
Mesorhizobium sp. OD48
Mesorhizobium sp. ORS 3324
Mesorhizobium sp. ORS 3355
Mesorhizobium sp. ORS 3356
Mesorhizobium sp. ORS 3359
Mesorhizobium sp. ORS 3365
Mesorhizobium sp. ORS 3369
Mesorhizobium sp. ORS 3404
Mesorhizobium sp. ORS 3428
Mesorhizobium sp. PA-5-Praia do Alemao
Mesorhizobium sp. PA-6-Praia do Alemao
Mesorhizobium sp. R-45553
Mesorhizobium sp. R-45615
Mesorhizobium sp. R-45754
Mesorhizobium sp. R-45755
Mesorhizobium sp. R-45768
Mesorhizobium sp. R-45817
Mesorhizobium sp. R-45834
Mesorhizobium sp. R-45843
Mesorhizobium sp. R-45863
Mesorhizobium sp. R-45975
Mesorhizobium sp. R-45976
Mesorhizobium sp. R-45994
Mesorhizobium sp. R-46042
Mesorhizobium sp. R-46043
Mesorhizobium sp. R-46044
Mesorhizobium sp. R-46045
Mesorhizobium sp. R-46071
Mesorhizobium sp. R-46074
Mesorhizobium sp. R-46108
Mesorhizobium sp. R-46110
Mesorhizobium sp. R-46209
Mesorhizobium sp. R-46321
Mesorhizobium sp. R-46328
Mesorhizobium sp. RITF1206
Mesorhizobium sp. RITF1212
Mesorhizobium sp. RITF1213
Mesorhizobium sp. RITF1214
Mesorhizobium sp. RITF1215
Mesorhizobium sp. RITF1217
Mesorhizobium sp. RITF1218
Mesorhizobium sp. RITF1564
Mesorhizobium sp. RITF711
Mesorhizobium sp. RITF712
Mesorhizobium sp. RITF721
Mesorhizobium sp. RITF731
Mesorhizobium sp. RITF742
Mesorhizobium sp. RITF902
Mesorhizobium sp. RITF903
Mesorhizobium sp. RITF905
Mesorhizobium sp. RITF907
Mesorhizobium sp. RITF908
Mesorhizobium sp. RP14(2022)
Mesorhizobium sp. Root552
Mesorhizobium sp. Root695
Mesorhizobium sp. S-26-Sintra
Mesorhizobium sp. S1274
Mesorhizobium sp. S1302
Mesorhizobium sp. S673
Mesorhizobium sp. S786
Mesorhizobium sp. S789
Mesorhizobium sp. S792
Mesorhizobium sp. S828
Mesorhizobium sp. S948
Mesorhizobium sp. S956
Mesorhizobium sp. SARCC-RB16n
Mesorhizobium sp. SBB-POLY
Mesorhizobium sp. SCAU11
Mesorhizobium sp. SCAU202A
Mesorhizobium sp. SCAU215
Mesorhizobium sp. SCAU2
Mesorhizobium sp. SCAU5
Mesorhizobium sp. SCAU9
Mesorhizobium sp. SCAUd6
Mesorhizobium sp. SEMIA 3007
Mesorhizobium sp. SL-1-Salir
Mesorhizobium sp. SL-2-Salir
Mesorhizobium sp. SL-5-Salir
Mesorhizobium sp. SL-7-Salir
Mesorhizobium sp. SL-9-Salir
Mesorhizobium sp. SOD10
Mesorhizobium sp. ST-5-Setubal
Mesorhizobium sp. STM 4661
Mesorhizobium sp. STM 8089
Mesorhizobium sp. STR-2-Santarem
Mesorhizobium sp. TUTAHSA144
Mesorhizobium sp. TUTAHSA150
Mesorhizobium sp. TUTAHSA41
Mesorhizobium sp. TUTAHSA45
Mesorhizobium sp. TUTAHSA57
Mesorhizobium sp. U510
Mesorhizobium sp. USDA 3350
Mesorhizobium sp. USDA 4207
Mesorhizobium sp. USDA 4214
Mesorhizobium sp. USDA 4236
Mesorhizobium sp. USDA 4255
Mesorhizobium sp. USDA 4297
Mesorhizobium sp. USDA 4318
Mesorhizobium sp. USDA 4322
Mesorhizobium sp. USDA-HM6
Mesorhizobium sp. WSM1283
Mesorhizobium sp. WSM1284
Mesorhizobium sp. WSM1497
Mesorhizobium sp. WSM2074
Mesorhizobium sp. WSM2561
Mesorhizobium sp. WSM3224
Mesorhizobium sp. WSM3626
Mesorhizobium sp. WSM3859
Mesorhizobium sp. WSM3860
Mesorhizobium sp. WSM3862
Mesorhizobium sp. WSM3864
Mesorhizobium sp. WSM3865
Mesorhizobium sp. WSM3866
Mesorhizobium sp. WSM3868
Mesorhizobium sp. WSM3869
Mesorhizobium sp. WSM3871
Mesorhizobium sp. WSM3872
Mesorhizobium sp. WSM3873
Mesorhizobium sp. WSM3875
Mesorhizobium sp. WSM3876
Mesorhizobium sp. WSM3877
Mesorhizobium sp. WSM3879
Mesorhizobium sp. WSM3882
Mesorhizobium sp. WSM3883
Mesorhizobium sp. WSM4303
Mesorhizobium sp. WSM4304
Mesorhizobium sp. WSM4305
Mesorhizobium sp. WSM4306
Mesorhizobium sp. WSM4307
Mesorhizobium sp. WSM4308
Mesorhizobium sp. WSM4310
Mesorhizobium sp. WSM4311
Mesorhizobium sp. WSM4312
Mesorhizobium sp. WSM4313
Mesorhizobium sp. WSM4315
Mesorhizobium sp. X874D
Mesorhizobium sp. X96B1
Mesorhizobium sp. YM1C-6-2
Mesorhizobium sp. rob10
Mesorhizobium sp. rob11
Mesorhizobium sp. rob12
Mesorhizobium sp. rob13
Mesorhizobium sp. rob6
Mesorhizobium sp. xhj20
Mesorhizobium sp. xhj23
Mesorhizobium sp. xhj26
Mesorhizobium sp. xhj7
Mesorhizobium sp.
Mesorhizobium tamadayense
Mesorhizobium tarimense
Mesorhizobium temperatum
Mesorhizobium tianshanense
Mesorhizobium ventifaucium
Mesorhizobium waimense
Mesorhizobium waitakense
Mesorhizobium wenxiniae
Mesorhizobium zhangyense
Mesorhizobium
Mesosutterella sp. AGMB02718
Mesosutterella sp. OilRF-GAM-744-9
Mesotoga infera
Mesotoga prima
Mesotoga sp. B105.6.4
Mesotoga sp. BH458_6_3_2_1
Mesotoga sp. Brook.08.YT.4.2.5.1
Mesotoga sp. H07.pep.5.3
Mesotoga sp. H07pep.5.4
Mesotoga sp. SC_3PWM13N19
Mesotoga sp. SC_4PWA21
Mesotoga sp. SC_NapDC3
Mesotoga sp. SC_NapDC
Mesotoga sp. TolDC
Mesotoga
Metabacillus bambusae
Metabacillus indicus LMG 22858
Metabacillus indicus
Metabacillus sp. KUDC1714
Metakosakonia sp. MRY16-398
Metallococcus carri
Metalysinibacillus saudimassiliensis
Methanimicrococcus blatticola
Methanobacteriaceae archaeon 41_258
Methanobacteriales archaeon HGW-Methanobacteriales-1
Methanobacteriales archaeon HGW-Methanobacteriales-2
Methanobacterium aggregans
Methanobacterium alcaliphilum
Methanobacterium alkalithermotolerans
Methanobacterium bryantii
Methanobacterium congolense
Methanobacterium formicicum DSM 3637
Methanobacterium formicicum
Methanobacterium ivanovii
Methanobacterium lacus
Methanobacterium paludis
Methanobacterium petrolearium
Methanobacterium sp. 42_16
Methanobacterium sp. A39
Methanobacterium sp. BAmetb5
Methanobacterium sp. BRmetb2
Methanobacterium sp. ERen5
Methanobacterium sp. MB1
Methanobacterium sp. MZ-A1
Methanobacterium sp. Maddingley MBC34
Methanobacterium sp. PtaB.Bin024
Methanobacterium sp. PtaU1.Bin097
Methanobacterium sp. SMA-27
Methanobacterium sp. VT
Methanobacterium sp.
Methanobacterium subterraneum
Methanobacterium
Methanobrevibacter arboriphilus JCM 13429 = DSM 1125
Methanobrevibacter arboriphilus
Methanobrevibacter curvatus
Methanobrevibacter cuticularis
Methanobrevibacter filiformis
Methanobrevibacter gottschalkii DSM 11977
Methanobrevibacter gottschalkii
Methanobrevibacter millerae
Methanobrevibacter olleyae
Methanobrevibacter ruminantium M1
Methanobrevibacter smithii ATCC 35061
Methanobrevibacter smithii CAG: 186
Methanobrevibacter smithii DSM 2374
Methanobrevibacter smithii DSM 2375
Methanobrevibacter sp. 87.7
Methanobrevibacter sp. NOE
Methanobrevibacter sp. TMH8
Methanobrevibacter wolinii
Methanocalculus chunghsingensis
Methanocalculus sp. 52_23
Methanocaldococcus bathoardescens
Methanocaldococcus fervens AG86
Methanocaldococcus fervens
Methanocaldococcus infernus ME
Methanocaldococcus infernus
Methanocaldococcus jannaschii DSM 2661
Methanocaldococcus jannaschii
Methanocaldococcus lauensis
Methanocaldococcus sp. FS406-22
Methanocaldococcus villosus KIN24-T80
Methanocaldococcus villosus
Methanocaldococcus vulcanius M7
Methanocaldococcus vulcanius
Methanocella arvoryzae MRE50
Methanocella arvoryzae
Methanocella conradii HZ254
Methanocella conradii
Methanocella paludicola SANAE
Methanocella paludicola
Methanocella sp. CWC-04
Methanococcoides burtonii DSM 6242
Methanococcoides burtonii
Methanococcoides methylutens MM1
Methanococcoides methylutens
Methanococcoides sp. AM1
Methanococcoides sp. NM1
Methanococcoides vulcani
Methanococcoides
Methanococcus aeolicus Nankai-3
Methanococcus aeolicus
Methanococcus maripaludis C5
Methanococcus maripaludis KA1
Methanococcus maripaludis OS7
Methanococcus maripaludis S2
Methanococcus maripaludis X1
Methanococcus maripaludis
Methanococcus vannielii SB
Methanococcus vannielii
Methanococcus voltae
Methanocorpusculum bavaricum
Methanocorpusculum labreanum Z
Methanocorpusculum labreanum
Methanocorpusculum parvum
Methanocorpusculum sp. GPch4
Methanocorpusculum sp. MCE
Methanocorpusculum sp. MG
Methanocorpusculum
Methanoculleus bourgensis MS2
Methanoculleus bourgensis
Methanoculleus chikugoensis
Methanoculleus marisnigri JR1
Methanoculleus marisnigri
Methanoculleus sediminis
Methanoculleus sp. CAG: 1088
Methanoculleus sp. MH98A
Methanoculleus sp. SDB
Methanoculleus taiwanensis
Methanoculleus thermophilus
Methanoculleus
Methanofervidicoccus abyssi
Methanofervidicoccus sp. A16
Methanofollis fontis
Methanofollis liminatans DSM 4140
Methanofollis liminatans
Methanogenium organophilum
Methanogenium sp. MK-MG
Methanohalobium evestigatum Z-7303
Methanohalobium evestigatum
Methanohalophilus euhalobius
Methanohalophilus halophilus
Methanohalophilus mahii DSM 5219
Methanohalophilus portucalensis FDF-1
Methanohalophilus sp. 2-GBenrich
Methanohalophilus sp. T328-1
Methanohalophilus sp. WG1-DM
Methanohalophilus sp.
Methanolacinia paynteri
Methanolacinia petrolearia DSM 11571
Methanolacinia petrolearia
Methanolacinia
Methanolinea mesophila
Methanolinea sp. SDB
Methanolinea sp.
Methanolobus bombayensis
Methanolobus chelungpuianus
Methanolobus halotolerans
Methanolobus profundi
Methanolobus psychrophilus R15
Methanolobus psychrotolerans
Methanolobus sp. T82-4
Methanolobus tindarius DSM 2278
Methanolobus tindarius
Methanolobus vulcani
Methanolobus zinderi
Methanomassiliicoccaceae archaeon DOK
Methanomassiliicoccales archaeon Mx-03
Methanomassiliicoccales archaeon PtaB.Bin134
Methanomassiliicoccales archaeon PtaU1.Bin030
Methanomassiliicoccales archaeon RumEn M1
Methanomassiliicoccales archaeon RumEn M2
Methanomassiliicoccus luminyensis
Methanomethylovorans hollandica DSM 15978
Methanomethylovorans sp. PtaU1.Bin073
Methanomethylovorans sp. PtaU1.Bin093
Methanomicrobiales archaeon 53_19
Methanomicrobiales archaeon HGW-Methanomicrobiales-1
Methanomicrobiales archaeon HGW-Methanomicrobiales-2
Methanomicrobiales archaeon HGW-Methanomicrobiales-3
Methanomicrobiales archaeon HGW-Methanomicrobiales-4
Methanomicrobiales archaeon HGW-Methanomicrobiales-6
Methanomicrobium mobile
Methanomicrobium sp. W14
Methanonatronarchaeum thermophilum
Methanophagales archaeon
Methanoplanus limicola DSM 2279
Methanoplanus limicola
Methanopyrus kandleri AV19
Methanopyrus kandleri
Methanopyrus sp. KOL6
Methanopyrus sp. SNP6
Methanoregula boonei 6A8
Methanoregula boonei
Methanoregula formicica SMSP
Methanoregula formicica
Methanoregula sp. PtaB.Bin085
Methanoregula sp. PtaU1.Bin006
Methanoregula sp. PtaU1.Bin051
Methanoregula sp.
Methanoregulaceae archaeon PtaB.Bin009
Methanoregulaceae archaeon PtaB.Bin056
Methanoregulaceae archaeon PtaB.Bin108
Methanoregulaceae archaeon PtaB.Bin152
Methanoregulaceae archaeon PtaU1.Bin066
Methanoregulaceae archaeon
Methanosaeta sp. ASM2
Methanosaeta sp. ASO1
Methanosaeta sp. NSM2
Methanosaeta sp. NSP1
Methanosaeta sp. PtaB.Bin018
Methanosaeta sp. PtaB.Bin087
Methanosaeta sp. PtaU1.Bin055
Methanosaeta sp. PtaU1.Bin060
Methanosaeta sp. PtaU1.Bin112
Methanosaeta sp. SDB
Methanosalsum zhilinae DSM 4017
Methanosalsum zhilinae
Methanosarcina acetivorans C2A
Methanosarcina acetivorans
Methanosarcina barkeri 227
Methanosarcina barkeri 3
Methanosarcina barkeri CM1
Methanosarcina barkeri MS
Methanosarcina barkeri str. Wiesmoor
Methanosarcina barkeri
Methanosarcina flavescens
Methanosarcina horonobensis HB-1 = JCM 15518
Methanosarcina horonobensis
Methanosarcina lacustris Z-7289
Methanosarcina lacustris
Methanosarcina mazei C16
Methanosarcina mazei Go1
Methanosarcina mazei LYC
Methanosarcina mazei S-6
Methanosarcina mazei SarPi
Methanosarcina mazei TMA
Methanosarcina mazei Tuc01
Methanosarcina mazei WWM610
Methanosarcina mazei
Methanosarcina siciliae C2J
Methanosarcina siciliae HI350
Methanosarcina siciliae T4/M
Methanosarcina siciliae
Methanosarcina soligelidi
Methanosarcina sp. 1.H.A.2.2
Methanosarcina sp. 1.H.T.1A.1
Methanosarcina sp. 2.H.A.1B.4
Methanosarcina sp. 795
Methanosarcina sp. A14
Methanosarcina sp. DH1
Methanosarcina sp. DH2
Methanosarcina sp. KYL-1
Methanosarcina sp. Kolksee
Methanosarcina sp. MSH10X1
Methanosarcina sp. MTP4
Methanosarcina sp. WH1
Methanosarcina sp. WWM596
Methanosarcina sp.
Methanosarcina spelaei
Methanosarcina spherical virus
Methanosarcina thermophila CHTI-55
Methanosarcina thermophila TM-1
Methanosarcina thermophila
Methanosarcina vacuolata Z-761
Methanosarcina vacuolata
Methanosarcina
Methanosarcinales archaeon ANME-1 ERB6
Methanosarcinales archaeon ANME-1 ERB7
Methanosarcinales archaeon ex4484_138
Methanosarcinales archaeon ex4572_44
Methanosarcinales archaeon
Methanosarcinales archaeon 56_1174
Methanosphaera sp. SHI1033
Methanosphaera stadtmanae DSM 3091
Methanosphaerula palustris E1-9c
Methanosphaerula palustris
Methanospirillum hungatei JF-1
Methanospirillum hungatei
Methanospirillum lacunae
Methanospirillum sp. J.3.6.1-F.2.7.3
Methanospirillum stamsii
Methanothermobacter defluvii
Methanothermobacter marburgensis str. Marburg
Methanothermobacter sp. EMTCatA1
Methanothermobacter sp. K4
Methanothermobacter sp. KEPCO-1
Methanothermobacter sp. THM-2
Methanothermobacter thermautotrophicus str. Delta H
Methanothermobacter thermautotrophicus
Methanothermobacter wolfeii
Methanothermobacter
Methanothermococcus okinawensis IH1
Methanothermococcus okinawensis
Methanothermococcus thermolithotrophicus
Methanothermus fervidus DSM 2088
Methanothrix harundinacea 6Ac
Methanothrix harundinacea
Methanothrix soehngenii GP6
Methanothrix sp.
Methanothrix thermoacetophila PT
Methanothrix
Methanotorris formicicus Mc-S-70
Methanotorris formicicus
Methanotorris igneus Kol 5
Methanotorris igneus
Methermicoccus shengliensis
Methylacidimicrobium cyclopophantes
Methylacidimicrobium sp. AP8
Methylacidimicrobium sp. B4
Methylacidimicrobium tartarophylax
Methylacidiphilum fumariolicum SolV
Methylacidiphilum fumariolicum
Methylacidiphilum infernorum V4
Methylacidiphilum infernorum
Methylacidiphilum kamchatkense Kam1
Methylacidiphilum kamchatkense
Methylacidiphilum sp. IT5
Methylacidiphilum sp. IT6
Methylacidiphilum sp. Phi
Methylacidiphilum sp. RTK17.1
Methylacidiphilum sp. Yel
Methylibium petroleiphilum PM1
Methylibium petroleiphilum
Methylibium sp. NZG
Methylibium sp. Root1272
Methylibium sp. T29-B
Methylibium sp. T29
Methylibium
Methylicorpusculum oleiharenae
Methylobacillus glycogenes
Methylobacillus methanolivorans
Methylobacter bovis
Methylobacter chroococcum
Methylobacter luteus
Methylobacter marinus
Methylobacter psychrophilus
Methylobacter sp. BlB1
Methylobacter sp. S3L5C
Methylobacter sp. YRD-M1
Methylobacter sp.
Methylobacter tundripaludum SV96
Methylobacter tundripaludum
Methylobacter vinelandii
Methylobacter
Methylobacterium adhaesivum
Methylobacterium aerolatum
Methylobacterium ajmalii
Methylobacterium aquaticum
Methylobacterium brachiatum
Methylobacterium brachythecii
Methylobacterium crusticola
Methylobacterium currus
Methylobacterium dankookense
Methylobacterium durans
Methylobacterium frigidaeris
Methylobacterium goesingense
Methylobacterium gossipiicola
Methylobacterium gregans
Methylobacterium haplocladii
Methylobacterium indicum
Methylobacterium iners
Methylobacterium isbiliense
Methylobacterium jeotgali
Methylobacterium longum
Methylobacterium marchantiae
Methylobacterium mesophilicum
Methylobacterium nodulans ORS 2060
Methylobacterium nodulans
Methylobacterium nonmethylotrophicum
Methylobacterium organophilum
Methylobacterium oryzae CBMB20
Methylobacterium oryzae
Methylobacterium persicinum
Methylobacterium phyllosphaerae
Methylobacterium phyllostachyos
Methylobacterium platani
Methylobacterium pseudosasicola
Methylobacterium radiodurans
Methylobacterium radiotolerans JCM 2831
Methylobacterium radiotolerans
Methylobacterium segetis
Methylobacterium soli
Methylobacterium sp. 10
Methylobacterium sp. 13MFTsu3.1M2
Methylobacterium sp. 174MFSha1.1
Methylobacterium sp. 17Sr1-1
Methylobacterium sp. 275MFSha3.1
Methylobacterium sp. 37f
Methylobacterium sp. 4-46
Methylobacterium sp. B1
Methylobacterium sp. B34
Methylobacterium sp. B4
Methylobacterium sp. C25
Methylobacterium sp. CG08_land_8_20_14_0_20_71_15
Methylobacterium sp. CG09_land_8_20_14_0_10_71_15
Methylobacterium sp. DB0501
Methylobacterium sp. DM1
Methylobacterium sp. E-005
Methylobacterium sp. E-045
Methylobacterium sp. E-046
Methylobacterium sp. E-065
Methylobacterium sp. FF17
Methylobacterium sp. GC_Met 2
Methylobacterium sp. GXF4
Methylobacterium sp. GXS13
Methylobacterium sp. J-026
Methylobacterium sp. J-030
Methylobacterium sp. J-059
Methylobacterium sp. J-067
Methylobacterium sp. J-068
Methylobacterium sp. J-070
Methylobacterium sp. J-072
Methylobacterium sp. J-076
Methylobacterium sp. J-077
Methylobacterium sp. J-078
Methylobacterium sp. J-088
Methylobacterium sp. J-090
Methylobacterium sp. L1A1
Methylobacterium sp. Leaf100
Methylobacterium sp. Leaf102
Methylobacterium sp. Leaf112
Methylobacterium sp. Leaf113
Methylobacterium sp. Leaf117
Methylobacterium sp. Leaf118
Methylobacterium sp. Leaf361
Methylobacterium sp. Leaf399
Methylobacterium sp. Leaf465
Methylobacterium sp. Leaf469
Methylobacterium sp. Leaf86
Methylobacterium sp. Leaf87
Methylobacterium sp. Leaf94
Methylobacterium sp. Leaf99
Methylobacterium sp. ME121
Methylobacterium sp. NMS14P
Methylobacterium sp. OT2
Methylobacterium sp. P1-11
Methylobacterium sp. PvR107
Methylobacterium sp. R2-1
Methylobacterium sp. SyP6R
Methylobacterium sp. UNC300MFChir4.1
Methylobacterium sp. UNC378MF
Methylobacterium sp. V23
Methylobacterium sp. WL103
Methylobacterium sp. WL116
Methylobacterium sp. WL12
Methylobacterium sp. WL18
Methylobacterium sp. WL69
Methylobacterium sp. WL8
Methylobacterium sp. WSM2598
Methylobacterium sp. XJLW
Methylobacterium sp. YIM 132548
Methylobacterium sp. ap11
Methylobacterium sp. yr596
Methylobacterium sp. yr668
Methylobacterium sp.
Methylobacterium tardum
Methylobacterium tarhaniae
Methylobacterium terrae
Methylobacterium terricola
Methylobacterium variabile
Methylobacterium
Methylocaldum marinum
Methylocaldum szegediense O-12
Methylocaldum szegediense
Methylocapsa acidiphila B2
Methylocapsa acidiphila
Methylocapsa aurea
Methylocapsa palsarum
Methyloceanibacter methanicus
Methyloceanibacter sp. wino2
Methyloceanibacter stevinii
Methylocella palustris
Methylocella silvestris BL2
Methylocella silvestris
Methylocella tundrae
Methylococcaceae bacterium AK-K6
Methylococcaceae bacterium BRS-K6
Methylococcaceae bacterium FWC3
Methylococcaceae bacterium NSM2-1
Methylococcaceae bacterium NSO1
Methylococcaceae bacterium NSP1-1
Methylococcaceae bacterium TMED69
Methylococcaceae bacterium
Methylococcales bacterium
Methylococcus capsulatus str. Bath
Methylococcus capsulatus str. Texas = ATCC 19069
Methylococcus capsulatus
Methylococcus geothermalis
Methylococcus sp. 16-5
Methylococcus sp. EFPC2
Methylococcus sp. LS7-MC
Methylococcus sp. Mc7
Methylococcus
Methylocucumis oryzae
Methylocystaceae bacterium
Methylocystaceae
Methylocystis bryophila
Methylocystis echinoides
Methylocystis heyeri
Methylocystis hirsuta
Methylocystis methanolicus
Methylocystis minimus
Methylocystis parvus OBBP
Methylocystis parvus
Methylocystis rosea SV97
Methylocystis rosea
Methylocystis silviterrae
Methylocystis sp. B8
Methylocystis sp. H15
Methylocystis sp. H4A
Methylocystis sp. H62
Methylocystis sp. H9a
Methylocystis sp. L43
Methylocystis sp. LW2
Methylocystis sp. LW5
Methylocystis sp. MJC1
Methylocystis sp. MitZ-2018
Methylocystis sp. SB2
Methylocystis sp. SC2
Methylocystis sp. SS37A-Re
Methylocystis sp. Sn-Cys
Methylocystis sp. m261
Methylocystis sp.
Methylocystis
Methyloferula stellata AR4
Methyloferula stellata
Methylogaea oryzae JCM 16910
Methylogaea oryzae
Methyloglobulus morosus KoM1
Methyloglobulus morosus
Methylomagnum ishizawai
Methylomarinum vadi
Methylomicrobium agile
Methylomicrobium lacus
Methylomicrobium sp. RS1
Methylomicrobium sp. wino1
Methylomicrobium
Methylomonas albis
Methylomonas denitrificans
Methylomonas fluvii
Methylomonas koyamae
Methylomonas lenta
Methylomonas methanica MC09
Methylomonas methanica
Methylomonas paludis
Methylomonas rhizoryzae
Methylomonas rubra
Methylomonas sp. 11b
Methylomonas sp. DH-1
Methylomonas sp. EFPC1
Methylomonas sp. Kb3
Methylomonas sp. LL1
Methylomonas sp. LW13
Methylomonas sp. LW15
Methylomonas sp. MK1
Methylomonas sp. R-45362
Methylomonas sp. R-45363
Methylomonas sp. R-45364
Methylomonas sp. R-45371
Methylomonas sp. R-45372
Methylomonas sp. R-45374
Methylomonas sp. R-45378
Methylomonas sp. R-45383
Methylomonas sp. R-49799
Methylomonas sp. R-49807
Methylomonas sp. SURF-1
Methylomonas sp. SURF-2
Methylomonas sp. WSC-6
Methylomonas sp. WSC-7
Methylomonas sp. ZR1
Methylomonas sp.
Methylomonas
Methylomusa anaerophila
Methylophaga aminisulfidivorans MP
Methylophaga frappieri
Methylophaga lonarensis MPL
Methylophaga lonarensis
Methylophaga thiooxydans DMS010
Methylophaga thiooxydans
Methylophaga
Methylophilaceae bacterium 17-44-8
Methylophilaceae bacterium
Methylophilales bacterium 16-45-7
Methylophilales bacterium 28-44-11
Methylophilus rhizosphaerae
Methylophilus sp. 13
Methylophilus sp.
Methyloprofundus sedimenti
Methylorubrum aminovorans
Methylorubrum extorquens CM4
Methylorubrum extorquens DSM 13060
Methylorubrum extorquens PA1
Methylorubrum extorquens
Methylorubrum podarium
Methylorubrum populi BJ001
Methylorubrum populi
Methylorubrum rhodesianum
Methylorubrum salsuginis
Methylorubrum sp. B1-46
Methylorubrum sp. GM97
Methylorubrum thiocyanatum
Methylorubrum zatmanii
Methylorubrum
Methylosinus sp. 3S-1
Methylosinus sp. B4S
Methylosinus sp. C49
Methylosinus sp. Ce-a6
Methylosinus sp. LW3
Methylosinus sp. LW4
Methylosinus sp. LW8
Methylosinus sp. PW1
Methylosinus sp. R-45379
Methylosinus sp. SN0510
Methylosinus sp. sav-2
Methylosinus sporium
Methylosinus trichosporium OB3b
Methylosinus trichosporium
Methylosinus
Methylosoma difficile
Methylosoma sp. TFB
Methylotenera mobilis
Methylotenera sp.
Methylotenera versatilis
Methyloterricola oryzae
Methylotetracoccus oryzae
Methylothermaceae bacteria B42
Methylotuvimicrobium alcaliphilum
Methylotuvimicrobium buryatense
Methyloversatilis discipulorum
Methyloversatilis sp. 12-65-5
Methyloversatilis sp. RAC08
Methyloversatilis sp. XJ19-13
Methyloversatilis sp. XJ19-49
Methyloversatilis thermotolerans
Methyloversatilis universalis
Methylovirgula ligni
Methylovirgula sp. HY1
Methylovorus glucosotrophus
Methylovulum miyakonense HT12
Methylovulum miyakonense
Methylovulum psychrotolerans
Methylovulum sp.
Micavibrio aeruginosavorus ARL-13
Micavibrio aeruginosavorus EPB
Micavibrio aeruginosavorus
Microbacteriaceae
Microbacterium aquimaris
Microbacterium arborescens
Microbacterium atlanticum
Microbacterium aurum
Microbacterium barkeri
Microbacterium bovistercoris
Microbacterium cremeum
Microbacterium dextranolyticum
Microbacterium enclense
Microbacterium esteraromaticum
Microbacterium flavum
Microbacterium gallinarum
Microbacterium halophytorum
Microbacterium halotolerans
Microbacterium helvum
Microbacterium hominis
Microbacterium hydrocarbonoxydans
Microbacterium keratanolyticum
Microbacterium kitamiense
Microbacterium kunmingensis
Microbacterium kyungheense
Microbacterium lacticum
Microbacterium laevaniformans
Microbacterium liquefaciens
Microbacterium lushaniae
Microbacterium luteum
Microbacterium mangrovi
Microbacterium marinum
Microbacterium maritypicum
Microbacterium mitrae
Microbacterium murale
Microbacterium oleivorans
Microbacterium oryzae
Microbacterium oxydans
Microbacterium paludicola
Microbacterium paraoxydans
Microbacterium paulum
Microbacterium phyllosphaerae
Microbacterium profundi
Microbacterium proteolyticum
Microbacterium pullorum
Microbacterium radiodurans
Microbacterium saperdae
Microbacterium schleiferi
Microbacterium sorbitolivorans
Microbacterium sp. 10M-3C3
Microbacterium sp. 16SH
Microbacterium sp. 2FI
Microbacterium sp. 3H14
Microbacterium sp. 8M
Microbacterium sp. ABRD_28
Microbacterium sp. AG238
Microbacterium sp. AG790
Microbacterium sp. AISO3
Microbacterium sp. Au-Mic1
Microbacterium sp. B19
Microbacterium sp. BH-3-3-3
Microbacterium sp. BK668
Microbacterium sp. BR1
Microbacterium sp. Be9
Microbacterium sp. Bi121
Microbacterium sp. Bi128
Microbacterium sp. C5A9
Microbacterium sp. CBA3102
Microbacterium sp. CFBP 13617
Microbacterium sp. CFH 31415
Microbacterium sp. CIAB417
Microbacterium sp. CJ77
Microbacterium sp. CSI-V
Microbacterium sp. EF45047
Microbacterium sp. EST19A
Microbacterium sp. F2E
Microbacterium sp. G2-8
Microbacterium sp. GCS4
Microbacterium sp. GbtcB3
Microbacterium sp. H37-C3
Microbacterium sp. HMWF026
Microbacterium sp. JC 701
Microbacterium sp. K27
Microbacterium sp. K35
Microbacterium sp. K41
Microbacterium sp. KSW4-10
Microbacterium sp. KSW4-16
Microbacterium sp. KSW4-6
Microbacterium sp. Leaf151
Microbacterium sp. Leaf161
Microbacterium sp. Leaf179
Microbacterium sp. Leaf320
Microbacterium sp. Leaf436
Microbacterium sp. MAH-37
Microbacterium sp. MRS-1
Microbacterium sp. MYb45
Microbacterium sp. MYb62
Microbacterium sp. MYb66
Microbacterium sp. MYb72
Microbacterium sp. Marseille-Q6648
Microbacterium sp. Mcb102
Microbacterium sp. NC79
Microbacterium sp. NE2HP2
Microbacterium sp. NFH-22A-Y
Microbacterium sp. No. 7
Microbacterium sp. ORS 1417
Microbacterium sp. ORS 1418
Microbacterium sp. ORS 1472
Microbacterium sp. OVT16B
Microbacterium sp. P26
Microbacterium sp. PI-1
Microbacterium sp. RU33B
Microbacterium sp. RURRCA19A
Microbacterium sp. Root1433D1
Microbacterium sp. Root61
Microbacterium sp. Ru50
Microbacterium sp. SA39
Microbacterium sp. SD291
Microbacterium sp. SGAir0570
Microbacterium sp. SL62
Microbacterium sp. SLBN-146
Microbacterium sp. SMR1
Microbacterium sp. SSW1-36
Microbacterium sp. SSW1-47
Microbacterium sp. SSW1-49
Microbacterium sp. Se5.02b
Microbacterium sp. T32
Microbacterium sp. TL13
Microbacterium sp. TNHR37B
Microbacterium sp. TPD7012
Microbacterium sp. TS-1
Microbacterium sp. UFMG61
Microbacterium sp. UMB0228
Microbacterium sp. USTB-Y
Microbacterium sp. VKM Ac-2870
Microbacterium sp. VKM Ac-2923
Microbacterium sp. W1N
Microbacterium sp. YJN-G
Microbacterium sp. YMB-B2
Microbacterium sp. Yaish 1
Microbacterium sp. ZOR0019
Microbacterium sp. ZXX196
Microbacterium sp. Zapt11
Microbacterium sp. cf046
Microbacterium sp. cf332
Microbacterium sp. kSW2-24
Microbacterium sp. nov. GSS16
Microbacterium sp. oral taxon 186
Microbacterium sp. p3-SID336
Microbacterium sp. zg.B185
Microbacterium sp. zg.B48
Microbacterium sp.
Microbacterium testaceum
Microbacterium thalassium
Microbacterium trichothecenolyticum
Microbacterium
Microbispora oryzae
Microbispora rosea
Microbispora sp. GKU 823
Microcella sp. MMS21-STM10
Micrococcales bacterium
Micrococcus cohnii
Micrococcus endophyticus
Micrococcus luteus
Micrococcus lylae
Micrococcus sp. FDAARGOS_333
Micrococcus sp. HG099
Micrococcus sp. HMSC067E09
Micrococcus sp. HMSC31B01
Micrococcus sp. JXJ CY 30
Micrococcus sp. KT16
Micrococcus sp. Mcc28
Micrococcus sp. SL257
Micrococcus sp. TA1
Micrococcus sp. Y70
Micrococcus yunnanensis
Micrococcus
Microcoleus asticus
Microcoleus sp. FACHB-1515
Microcoleus sp. FACHB-1
Microcoleus sp. FACHB-53
Microcoleus sp. FACHB-672
Microcoleus sp. FACHB-68
Microcoleus sp. FACHB-831
Microcoleus sp. FACHB-SPT15
Microcoleus sp. LEGE 07076
Microcoleus sp. PCC 8701
Microcoleus vaginatus FGP-2
Microcoleus vaginatus
Microcystis aeruginosa TAIHU98
Microcystis aeruginosa
Microcystis flos-aquae
Microcystis sp. 0824
Microcystis sp. LEGE 08355
Microcystis viridis
Microcystis wesenbergii
Microcystis
Microgenomates bacterium DG_75
Microgenomates group bacterium GW2011_GWA2_44_7
Microlunatus flavus
Microlunatus panaciterrae
Microlunatus sagamiharensis
Micromonas commoda
Micromonospora citrea
Micromonospora costi
Micromonospora coxensis
Micromonospora halophytica
Micromonospora jinlongensis
Micromonospora lupini str. Lupac 08
Micromonospora matsumotoense
Micromonospora nigra
Micromonospora pattaloongensis
Micromonospora peucetia
Micromonospora purpureochromogenes
Micromonospora radicis
Micromonospora saelicesensis
Micromonospora sp. A202
Micromonospora sp. C81
Micromonospora sp. HSS6-12
Micromonospora sp. M61
Micromonospora sp. M71_S20
Micromonospora sp. MH33
Micromonospora sp. MIA32
Micromonospora sp. MIA38
Micromonospora sp. MIA57
Micromonospora sp. R7-596
Micromonospora sp. RP3T
Micromonospora sp. SB1-39
Micromonospora sp. SB1-46
Micromonospora viridifaciens
Micromonospora wenchangensis
Micromonospora zamorensis
Micropruina sp. KQZ13P-5
Microscilla marina ATCC 23134
Microscilla marina
Microseira minor
Microseira wollei K1
Microseira wollei K2
Microseira wollei K3
Microseira wollei K4
Microseira wollei K5
Microseira wollei K6
Microseira wollei K7
Microseira wollei K8
Microseira wollei NIES-4236
Microseira wollei YC0404
Microseira wollei YC1109
Microseira wollei
Microvenator marinus
Microvirga aerophila
Microvirga antarctica
Microvirga arabica
Microvirga arsenatis
Microvirga calopogonii
Microvirga lotononidis
Microvirga lupini
Microvirga mediterraneensis
Microvirga ossetica
Microvirga pakistanensis
Microvirga pudoricolor
Microvirga rosea
Microvirga roseola
Microvirga soli
Microvirga sp. 3-52
Microvirga sp. BSC39
Microvirga sp. BT290
Microvirga sp. BT689
Microvirga sp. HBU65207
Microvirga sp. HBU67692
Microvirga sp. KLBC 81
Microvirga sp. R24
Microvirga sp. VF16
Microvirga sp. WGZ8
Microvirga sp.
Microvirga splendida
Microvirga subterranea
Microvirga thermotolerans
Microvirga tunisiensis
Microvirga vignae
Microvirga zambiensis WSM3693
Microvirga zambiensis
Microvirga
Millisia brevis
Miltoncostaea marina
Miltoncostaea oceani
Minwuia thermotolerans
Mitsuaria chitosanitabida
Mitsuaria sp. 7
Mitsuaria sp. TWR114
Mitsuokella jalaludinii
Mitsuokella multacida
Mitsuokella sp. AF21-1AC
Mitsuokella sp. AF33-22
Mitsuokella sp. oral taxon 131 str. W9106
Mitsuokella sp. oral taxon 131
Mixta calida
Mizugakiibacter sediminis
Mobilibacterium timonense
Mobilisporobacter senegalensis
Mobilitalea sibirica
Modestobacter altitudinis
Modestobacter caceresii
Modestobacter italicus
Modestobacter marinus
Modestobacter muralis
Modestobacter sp. DSM 44400
Modestobacter sp. KNN46-3
Modestobacter sp. L9-4
Modestobacter sp. Leaf380
Modestobacter sp. VKM Ac-2978
Modestobacter sp. VKM Ac-2983
Modestobacter sp. VKM Ac-2984
Modestobacter sp. VKM Ac-2985
Modestobacter versicolor
Mogibacterium diversum
Mogibacterium kristiansenii
Mogibacterium pumilum
Mogibacterium sp. CM50
Mogibacterium timidum ATCC 33093
Mogibacterium timidum
Mogibacterium
Mongoliimonas terrestris
Monoglobus pectinilyticus
Moorella glycerini
Moorella humiferrea
Moorella mulderi DSM 14980
Moorella mulderi
Moorella sp. 60_41
Moorella sp. E306M
Moorella sp. E308F
Moorella sp. Hama-1
Moorella stamsii
Moorella thermoacetica Y72
Moorella thermoacetica
Moorella
Moorena bouillonii
Moorena producens
Moraxella bovis
Mordavella massiliensis
Moritella dasanensis
Moritella sp. JT01
Moritella viscosa
Motilibacter rhizosphaerae
Motiliproteus coralliicola
Motiliproteus sediminis
Motiliproteus sp. SC1-56
Mucilaginibacter achroorhodeus
Mucilaginibacter agri
Mucilaginibacter aquatilis
Mucilaginibacter conchicola
Mucilaginibacter daejeonensis
Mucilaginibacter dorajii
Mucilaginibacter endophyticus
Mucilaginibacter frigoritolerans
Mucilaginibacter galii
Mucilaginibacter ginkgonis
Mucilaginibacter ginsenosidivorax
Mucilaginibacter gossypii
Mucilaginibacter gossypiicola
Mucilaginibacter gracilis
Mucilaginibacter hurinus
Mucilaginibacter jinjuensis
Mucilaginibacter kameinonensis
Mucilaginibacter myungsuensis
Mucilaginibacter oryzae
Mucilaginibacter pallidiroseus
Mucilaginibacter paludis
Mucilaginibacter pankratovii
Mucilaginibacter pedocola
Mucilaginibacter phyllosphaerae
Mucilaginibacter pineti
Mucilaginibacter polytrichastri
Mucilaginibacter rigui
Mucilaginibacter rivuli
Mucilaginibacter robiniae
Mucilaginibacter roseus
Mucilaginibacter rubeus
Mucilaginibacter segetis
Mucilaginibacter sp. 21P
Mucilaginibacter sp. Bleaf8
Mucilaginibacter sp. FT3.2
Mucilaginibacter sp. JRF
Mucilaginibacter sp. JXJ CY 39
Mucilaginibacter sp. KACC 22063
Mucilaginibacter sp. L196
Mucilaginibacter sp. MD40
Mucilaginibacter sp. PPCGB 2223
Mucilaginibacter sp. RS28
Mucilaginibacter sp. SG538B
Mucilaginibacter sp. SMC90
Mucilaginibacter sp. SP1R1
Mucilaginibacter sp. UR6-11
Mucilaginibacter sp. UR6-1
Mucilaginibacter sp. X4EP1
Mucilaginibacter terrenus
Mucilaginibacter terrigena
Mucilaginibacter xinganensis
Mucilaginibacter
Mucuna pruriens
Mumia flava
Mumia
Murdochiella vaginalis
Muricauda aequoris
Muricauda amoyensis
Muricauda aurantiaca
Muricauda pacifica
Muricauda sediminis
Muricauda sp. TY007
Muricomes intestini
Muriicola jejuensis
Murinocardiopsis flavida
Mus caroli
Musicola keenii
Musicola paradisiaca Ech703
Musicola paradisiaca
Musicola
Myceligenerans xiligouense
Mycetocola lacteus
Mycetocola reblochoni
Mycetocola saprophilus
Mycetocola sp. BIGb0189
Mycetocola sp. JXN-3
Mycobacteriaceae
Mycobacterium aquaticum
Mycobacterium avium subsp. paratuberculosis
Mycobacterium avium
Mycobacterium colombiense
Mycobacterium crocinum
Mycobacterium diernhoferi
Mycobacterium dioxanotrophicus
Mycobacterium frederiksbergense
Mycobacterium genavense
Mycobacterium goodii
Mycobacterium gordonae
Mycobacterium grossiae
Mycobacterium hackensackense
Mycobacterium haemophilus DSM 44634
Mycobacterium haemophilu
Mycobacterium helveticum
Mycobacterium hodleri
Mycobacterium kansasii 662
Mycobacterium kyogaense
Mycobacterium lentiflavum
Mycobacterium leprae
Mycobacterium lepromatosis
Mycobacterium marinum
Mycobacterium marseillense
Mycobacterium palauense
Mycobacterium paraffinicum
Mycobacterium paragordonae
Mycobacterium paraintracellulare
Mycobacterium paraseoulense
Mycobacterium porcinum
Mycobacterium pyrenivorans
Mycobacterium saskatchewanense
Mycobacterium scrofulaceum
Mycobacterium sp. 1245852.3
Mycobacterium sp. 141
Mycobacterium sp. 155
Mycobacterium sp. 3519A
Mycobacterium sp. 852002-51057_SCH5723018
Mycobacterium sp. 852014-52144_SCH5372336
Mycobacterium sp. ACS1612
Mycobacterium sp. AT1
Mycobacterium sp. DBP42
Mycobacterium sp. DL440
Mycobacterium sp. DL592
Mycobacterium sp. E1747
Mycobacterium sp. E2327
Mycobacterium sp. E2497
Mycobacterium sp. E2699
Mycobacterium sp. E2989
Mycobacterium sp. E787
Mycobacterium sp. E796
Mycobacterium sp. E802
Mycobacterium sp. GA-1841
Mycobacterium sp. GA-2829
Mycobacterium sp. IEC1808
Mycobacterium sp. IS-1742
Mycobacterium sp. ITM-2016-00317
Mycobacterium sp. ITM-2017-0098
Mycobacterium sp. IWGMT90018-18076
Mycobacterium sp. JS623
Mycobacterium sp. MS1601
Mycobacterium sp. NAZ190054
Mycobacterium sp. NS-7484
Mycobacterium sp. PS03-16
Mycobacterium sp. PSTR-4-N
Mycobacterium sp. QGD 101
Mycobacterium sp. Root135
Mycobacterium sp. Root265
Mycobacterium sp. SMC-4
Mycobacterium sp. UM_CSW
Mycobacterium sp. WY10
Mycobacterium sp. YC-RL4
Mycobacterium sp. shizuoka-1
Mycobacterium timonense
Mycobacterium tuberculosis variant bovis BCG
Mycobacterium tuberculosis
Mycobacterium ulcerans group
Mycobacterium ulcerans
Mycobacterium yunnanensis
Mycobacterium
Mycobacteroides abscessus subsp. abscessus
Mycobacteroides abscessus subsp. bolletii
Mycobacteroides abscessus
Mycobacteroides chelonae
Mycobacteroides franklinii
Mycobacteroides salmoniphilum
Mycobacteroides saopaulense
Mycobacteroides sp. LB1
Mycobacteroides
Mycolicibacter sp. MYC098
Mycolicibacter sp. MYC101
Mycolicibacter sp. MYC340
Mycolicibacterium agri
Mycolicibacterium aichiense
Mycolicibacterium alvei
Mycolicibacterium anyangense
Mycolicibacterium aromaticivorans
Mycolicibacterium aubagnense
Mycolicibacterium austroafricanum
Mycolicibacterium bacteremicum
Mycolicibacterium baixiangningiae
Mycolicibacterium boenickei
Mycolicibacterium brisbanense
Mycolicibacterium brumae
Mycolicibacterium canariasense
Mycolicibacterium chubuense
Mycolicibacterium conceptionense
Mycolicibacterium confluentis
Mycolicibacterium cosmeticum
Mycolicibacterium farcinogenes
Mycolicibacterium fluoranthenivorans
Mycolicibacterium fortuitum
Mycolicibacterium gilvum
Mycolicibacterium helvum
Mycolicibacterium hippocampi
Mycolicibacterium houstonense
Mycolicibacterium iranicum
Mycolicibacterium komanii
Mycolicibacterium komossense
Mycolicibacterium lacusdiani
Mycolicibacterium litorale
Mycolicibacterium llatzerense
Mycolicibacterium lutetiense
Mycolicibacterium madagascariense
Mycolicibacterium mageritense
Mycolicibacterium malmesburyense
Mycolicibacterium mengxianglii
Mycolicibacterium monacense
Mycolicibacterium moriokaense
Mycolicibacterium mucogenicum
Mycolicibacterium murale
Mycolicibacterium neoaurum
Mycolicibacterium neworleansense
Mycolicibacterium obuense
Mycolicibacterium parafortuitum
Mycolicibacterium peregrinum
Mycolicibacterium phlei
Mycolicibacterium phocaicum
Mycolicibacterium rhodesiae
Mycolicibacterium sarraceniae
Mycolicibacterium sediminis
Mycolicibacterium septicum
Mycolicibacterium setense
Mycolicibacterium smegmatis
Mycolicibacterium sp. 018/SC-01/001
Mycolicibacterium sp. BK634
Mycolicibacterium sp. BiH015
Mycolicibacterium sp. CAU 1645
Mycolicibacterium sp. CBMA 226
Mycolicibacterium sp. CBMA 234
Mycolicibacterium sp. CBMA 295
Mycolicibacterium sp. CBMA 361
Mycolicibacterium sp. CH28
Mycolicibacterium sp. CR10
Mycolicibacterium sp. J2
Mycolicibacterium sp. NCC-Tsukiji
Mycolicibacterium sp. P1-18
Mycolicibacterium sp. P9-22
Mycolicibacterium sp. P9-64
Mycolicibacterium sp. PAM1
Mycolicibacterium sp. S2-37
Mycolicibacterium sphagni
Mycolicibacterium stellerae
Mycolicibacterium tokaiense
Mycolicibacterium tusciae
Mycolicibacterium vanbaalenii
Mycolicibacterium vinylchloridicum
Mycolicibacterium wolinskyi
Mycolicibacterium xanthum
Mycolicibacterium
Mycoplasma putrefaciens
Myxacorys almedinensis
Myxococcaceae bacterium
Myxococcaceae
Myxococcus hansupus
Myxococcus sp. RHSTA-1-4
Myxococcus sp. SCHIC003
Myxococcus stipitatus
Myxococcus vastator
Myxococcus xanthus
Myxosarcina sp. GI1
Myxosarcina sp. PCC 7312
Naasia lichenicola
Naasia sp. SYSU D00057
Naegleria gruberi
Nafulsella turpanensis
Nakamurella flava
Nakamurella leprariae
Nakamurella panacisegetis
Nakamurella sp. YIM 132087
Nannocystis exedens
Nannocystis pusilla
Nannocystis sp. ILAH1
Nannocystis sp. RBIL2
Nannocystis sp. SCPEA4
Nannocystis sp. bb15-2
Nannocystis sp. fl3
Nannocystis sp. ncelm1
Natranaerobius thermophilus JW/NM-WN-LF
Natranaerobius thermophilus
Natranaerovirga hydrolytica
Natranaerovirga pectinivora
Natrarchaeobaculum aegyptiacum
Natrialba chahannaoensis JCM 10990
Natrialba chahannaoensis
Natrinema pallidum DSM 3751
Natrinema pallidum
Natronincola peptidivorans
Natronobacillus azotifigens
Natronoflexus pectinivorans
Natronolimnobius baerhuensis
Natronolimnobius sp. AArcel1
Natronomonas halophila
Natronomonas sp. ZY43
Natronorubrum texcoconense
Natronorubrum tibetense GA33
Natronorubrum tibetense
Natronospirillum operosum
Naumannella halotolerans
Nautilia profundicola AmH
Nautilia profundicola
Neiella holothuriorum
Neiella marina
Neiella sp. HB171785
Neisseria gonorrhoeae
Neisseria meningitidis
Neisseria mucosa ATCC 25996
Neobacillus bataviensis LMG 21833
Neobacillus bataviensis
Neobacillus cucumis
Neobacillus mesonae
Neobacillus niacini
Neobacillus vireti LMG 21834
Neobacillus vireti
Neobittarella massiliensis
Neochlamydia sp. S13
Neomonachus schauinslandi
Neorhizobium alkalisoli
Neorhizobium galegae bv. officinalis bv. officinalis str. HAMBI 1141
Neorhizobium galegae bv. officinalis
Neorhizobium galegae bv. orientalis str. HAMBI 540
Neorhizobium galegae bv. orientalis
Neorhizobium galegae
Neorhizobium huautlense
Neorhizobium petrolearium
Neorhizobium sp. JUb45
Neorhizobium sp. NCHU2750
Neorhizobium sp.
Neorhizobium vignae
Neosynechococcus sphagnicola sy1
Neosynechococcus sphagnicola
Neotabrizicola shimadae
Neowestiellopsis persica A1387
Neptuniibacter sp. Phe_28
Neptunomonas japonica
Neptunomonas marina
Neptunomonas sp. XY-337
Nereida ignava
Nesterenkonia alkaliphila
Nesterenkonia ebinurensis
Nesterenkonia haasae
Nesterenkonia massiliensis
Nesterenkonia pannonica
Nesterenkonia populi
Nesterenkonia salmonea
Nesterenkonia sedimenti
Nesterenkonia sp. GX14115
Nesterenkonia sp. NBAIMH1
Nesterenkonia sp. PF2B19
Nesterenkonia sphaerica
Nevskia ramosa
Nevskia soli
Niabella hibiscisoli
Niallia circulans
Niallia endozanthoxylica
Niallia nealsonii AAU1
Niallia nealsonii
Niallia sp. NCCP-28
Niameybacter massiliensis
Niastella koreensis
Niastella populi
Niastella yeongjuensis
Nibribacter ruber
Nibricoccus aquaticus
Nitratifractor salsuginis DSM 16511
Nitratireductor aquibiodomus
Nitratireductor aquimarinus
Nitratireductor basaltis
Nitratireductor indicus C115
Nitratiruptor tergarcus DSM 16512
Nitratiruptor tergarcus
Nitrincola alkalisediminis
Nitrincola iocasae
Nitrincola lacisaponensis
Nitrincola nitratireducens
Nitrincola schmidtii
Nitrincola sp. A-D6
Nitrincola tapanii
Nitrincola
Nitritalea halalkaliphila
Nitrobacter vulgaris
Nitrobacteraceae
Nitrococcus mobilis Nb-231
Nitrococcus mobilis
Nitrogeniibacter aestuarii
Nitrogeniibacter mangrovi
Nitrolancea hollandica
Nitrosococcus halophilus Nc 4
Nitrosococcus oceani
Nitrosomonas aestuarii
Nitrosomonas marina
Nitrosomonas oligotropha
Nitrososphaera sp.
Nitrospina gracilis 3/211
Nitrospina gracilis
Nitrospira bacterium SG8_35_1
Nitrospira bacterium SG8_35_4
Nitrospira bacterium SG_3
Nitrospira bacterium SM23_35
Nitrospira cf. moscoviensis SBR1015
Nitrospira defluvii
Nitrospira japonica
Nitrospira moscoviensis
Nitrospira sp. HN-bin3
Nitrospira sp. ND1
Nitrospira sp. OLB3
Nitrospira sp. SG-bin1
Nitrospira sp. UW-LDO-01
Nitrospiraceae bacterium
Nitrospirae bacterium CG02_land_8_20_14_3_00_41_53
Nitrospirae bacterium CG06_land_8_20_14_3_00_70_43
Nitrospirae bacterium CG08_land_8_20_14_0_20_52_24
Nitrospirae bacterium CG11_big_fil_rev_8_21_14_0_20_41_14
Nitrospirae bacterium CG17_big_fil_post_rev_8_21_14_2_50_50_9
Nitrospirae bacterium CG18_big_fil_WC_8_21_14_2_50_70_55
Nitrospirae bacterium CG2_30_41_42
Nitrospirae bacterium CG2_30_70_394
Nitrospirae bacterium CG_4_10_14_0_8_um_filter_41_2_3
Nitrospirae bacterium CG_4_10_14_3_um_filter_53_41
Nitrospirae bacterium CG_4_10_14_3_um_filter_70_108
Nitrospirae bacterium CG_4_8_14_3_um_filter_41_47
Nitrospirae bacterium CG_4_8_14_3_um_filter_50_41
Nitrospirae bacterium CG_4_8_14_3_um_filter_70_85
Nitrospirae bacterium CG_4_9_14_0_8_um_filter_70_14
Nitrospirae bacterium CG_4_9_14_3_um_filter_41_27
Nitrospirae bacterium CG_4_9_14_3_um_filter_53_35
Nitrospirae bacterium RIFOXYA2_FULL_44_9
Nitrospirillum amazonense CBAmc
Nitrospirillum amazonense Y2
Nitrospirillum amazonense
Nitrospirillum iridis
Nitrospirota bacterium
Niveibacterium microcysteis
Niveibacterium sp. 24ML
Niveibacterium sp. COAC-50
Niveibacterium umoris
Niveibacterium
Niveispirillum cyanobacteriorum
Niveispirillum irakense DSM 11586
Niveispirillum irakense
Niveispirillum lacus
Niveispirillum sp. SYP-B3756
Nocardia abscessus
Nocardia acididurans
Nocardia acidivorans
Nocardia africana
Nocardia alba
Nocardia altamirensis
Nocardia amamiensis
Nocardia amikacinitolerans
Nocardia anaemiae
Nocardia araoensis
Nocardia arizonensis
Nocardia asiatica
Nocardia asteroides
Nocardia aurantia
Nocardia aurantiaca
Nocardia aurea
Nocardia beijingensis
Nocardia bhagyanarayanae
Nocardia bovistercoris
Nocardia brasiliensis ATCC 700358
Nocardia brasiliensis
Nocardia cerradoensis
Nocardia colli
Nocardia concava
Nocardia crassostreae
Nocardia cyriacigeorgica
Nocardia donostiensis
Nocardia elegans
Nocardia farcinica
Nocardia fluminea
Nocardia gipuzkoensis
Nocardia higoensis
Nocardia huaxiensis
Nocardia inohanensis
Nocardia jiangxiensis
Nocardia lijiangensis
Nocardia mangyaensis
Nocardia mexicana
Nocardia miyunensis
Nocardia neocaledoniensis
Nocardia niigatensis
Nocardia ninae
Nocardia noduli
Nocardia nova
Nocardia otitidiscaviarum
Nocardia pneumoniae
Nocardia pseudobrasiliensis
Nocardia pseudovaccinii
Nocardia puris
Nocardia rhizosphaerihabitans
Nocardia rosealba
Nocardia salmonicida
Nocardia seriolae
Nocardia shimofusensis
Nocardia sp. BMG111209
Nocardia sp. CNY236
Nocardia sp. CS682
Nocardia sp. DSM 111726
Nocardia sp. DSM 111727
Nocardia sp. MH4
Nocardia sp. NBC_00802
Nocardia sp. NRRL S-836
Nocardia sp. NRRL WC-3656
Nocardia sp. Root136
Nocardia sp. alder85J
Nocardia sputi
Nocardia stercoris
Nocardia suismassiliense
Nocardia tenerifensis
Nocardia tengchongensis
Nocardia terpenica
Nocardia terrae
Nocardia uniformis
Nocardia veterana
Nocardia vulneris
Nocardia xishanensis
Nocardia yamanashiensis
Nocardia yunnanensis
Nocardia
Nocardioides acrostichi
Nocardioides albertanoniae
Nocardioides albidus
Nocardioides alkalitolerans
Nocardioides alpinus
Nocardioides anomalus
Nocardioides aquaticus
Nocardioides aromaticivorans
Nocardioides bruguierae
Nocardioides caeni
Nocardioides cavernae
Nocardioides currus
Nocardioides daeguensis
Nocardioides daejeonensis
Nocardioides dokdonensis
Nocardioides dongxiaopingii
Nocardioides flavescens
Nocardioides ganghwensis
Nocardioides glacieisoli
Nocardioides guangzhouensis
Nocardioides halotolerans
Nocardioides humi
Nocardioides humilatus
Nocardioides immobilis
Nocardioides iriomotensis
Nocardioides jensenii
Nocardioides kongjuensis
Nocardioides lacusdianchii
Nocardioides litoris
Nocardioides luteus
Nocardioides mangrovi
Nocardioides marinisabuli
Nocardioides marinus
Nocardioides massiliensis
Nocardioides nitrophenolicus
Nocardioides okcheonensis
Nocardioides palaemonis
Nocardioides panacisoli
Nocardioides pelophilus
Nocardioides phosphati
Nocardioides piscis
Nocardioides psychrotolerans
Nocardioides rotundus
Nocardioides rubriscoriae
Nocardioides salarius
Nocardioides sediminis
Nocardioides sp. 1609
Nocardioides sp. AX2bis
Nocardioides sp. B-3
Nocardioides sp. CCBAU 10876
Nocardioides sp. CF8
Nocardioides sp. CFH 31398
Nocardioides sp. DJM-14
Nocardioides sp. GY 10113
Nocardioides sp. HDW12B
Nocardioides sp. J54
Nocardioides sp. J9
Nocardioides sp. KC13
Nocardioides sp. Kera G14
Nocardioides sp. OK12
Nocardioides sp. Root190
Nocardioides sp. S-1144
Nocardioides sp. S5
Nocardioides sp. SLBN-35
Nocardioides sp. STR3
Nocardioides sp. Soil777
Nocardioides sp. Soil796
Nocardioides sp. Soil805
Nocardioides sp. WS12
Nocardioides sp. YR527
Nocardioides sp. zg-1230
Nocardioides stalactiti
Nocardioides terrigena
Nocardioides ungokensis
Nocardioides zeae
Nocardioides zhouii
Nocardioides
Nocardiopsaceae bacterium YIM 96095
Nocardiopsis aegyptia
Nocardiopsis alba
Nocardiopsis alborubida
Nocardiopsis algeriensis
Nocardiopsis alkaliphila
Nocardiopsis chromatogenes
Nocardiopsis composta
Nocardiopsis coralli
Nocardiopsis dassonvillei
Nocardiopsis deserti
Nocardiopsis exhalans
Nocardiopsis flavescens
Nocardiopsis ganjiahuensis
Nocardiopsis kunsanensis
Nocardiopsis listeri
Nocardiopsis lucentensis
Nocardiopsis metallicus
Nocardiopsis potens
Nocardiopsis prasina
Nocardiopsis quinghaiensis
Nocardiopsis salina
Nocardiopsis sinuspersici
Nocardiopsis sp. B62
Nocardiopsis sp. CNR-923
Nocardiopsis sp. CNS-639
Nocardiopsis sp. CNT312
Nocardiopsis sp. EMB25
Nocardiopsis sp. FIRDI 009
Nocardiopsis sp. HDS12
Nocardiopsis sp. Huas11
Nocardiopsis sp. JB363
Nocardiopsis sp. L17-MgMaSL7
Nocardiopsis sp. LSu2-4
Nocardiopsis sp. MG754419
Nocardiopsis sp. NRRL B-16309
Nocardiopsis sp. RSe5-2
Nocardiopsis sp. RV163
Nocardiopsis sp. SBT366
Nocardiopsis sp. TNDT3
Nocardiopsis sp. TSRI0078
Nocardiopsis terrae
Nocardiopsis valliformis
Nocardiopsis xinjiangensis
Nocardiopsis
Nodosilinea nodulosa PCC 7104
Nodosilinea nodulosa UTEX B 2910
Nodosilinea nodulosa
Nodosilinea sp. FACHB-131
Nodosilinea sp. FACHB-13
Nodosilinea sp. FACHB-141
Nodosilinea sp. LEGE 06152
Nodosilinea sp. LEGE 07088
Nodosilinea sp. LEGE 07298
Nodosilinea sp. P-1105
Nodosilinea sp. T6-1119
Nodularia harveyana CCAP 1452/1
Nodularia sp. (in: cyanobacteria)
Nodularia sp. 10KLT1
Nodularia sp. KAC 13
Nodularia sp. NIES-3585
Nodularia sphaerocarpa Up16a
Nodularia sphaerocarpa Up16f
Nodularia sphaerocarpa
Nodularia spumigena AV1
Nodularia spumigena CCY9414
Nodularia spumigena FL2f
Nodularia spumigena UHCC 0039
Nodularia spumigena
Nodularia
Nonlabens ulvanivorans
Nonomuraea africana
Nonomuraea angiospora
Nonomuraea antri
Nonomuraea aridisoli
Nonomuraea deserti
Nonomuraea diastatica
Nonomuraea dietziae
Nonomuraea fuscirosea
Nonomuraea gerenzanensis
Nonomuraea jiangxiensis
Nonomuraea rubra
Nonomuraea sp. ATCC 55076
Nonomuraea sp. FMUSA5-5
Nonomuraea sp. MCN248
Nonomuraea sp. PA05
Nonomuraea sp. SBT364
Nonomuraea sp. WAC 01424
Nonomuraea spiralis
Nonomuraea thailandensis
Nonomuraea turkmeniaca
Nostoc calcicola CG9
Nostoc calcicola Ind30
Nostoc calcicola Ind31
Nostoc calcicola Ind32
Nostoc calcicola
Nostoc carneum CG7
Nostoc carneum NIES-2107
Nostoc cf. commune SO-36
Nostoc commune CG3
Nostoc commune CP
Nostoc commune HK-02
Nostoc commune NIES-4072
Nostoc commune SC
Nostoc commune
Nostoc cycadae WK-1
Nostoc cycadae
Nostoc edaphicum CCNP1411
Nostoc edaphicum
Nostoc ellipsosporum NDUPC002
Nostoc favosum
Nostoc flagelliforme CCNUN1
Nostoc flagelliforme NX-09
Nostoc flagelliforme
Nostoc foliaceum
Nostoc linckia CG5
Nostoc linckia z13
Nostoc linckia z14
Nostoc linckia z15
Nostoc linckia z16
Nostoc linckia z18
Nostoc linckia z1
Nostoc linckia z2
Nostoc linckia z3
Nostoc linckia z4
Nostoc linckia z6
Nostoc linckia z7
Nostoc linckia z8
Nostoc linckia z9
Nostoc linckia
Nostoc minutum NIES-26
Nostoc mirabile
Nostoc parmelioides
Nostoc piscinale CENA21
Nostoc piscinale
Nostoc punctiforme CG1
Nostoc punctiforme NIES-2108
Nostoc punctiforme PCC 73102
Nostoc punctiforme
Nostoc sp. ‘J. Gallon’
Nostoc sp. ‘Lobaria pulmonaria (5183) cyanobiont’
Nostoc sp. ‘Peltigera malacea cyanobiont’ DB3992
Nostoc sp. ‘Peltigera membranacea cyanobiont’ 210A
Nostoc sp. ‘Peltigera membranacea cyanobiont’ 213
Nostoc sp. ‘Peltigera membranacea cyanobiont’ 232
Nostoc sp. ‘Peltigera membranacea cyanobiont’ N6
Nostoc sp. ‘Peltigera ponojensis photobiont’
Nostoc sp. 106C
Nostoc sp. 1189P
Nostoc sp. 1190P
Nostoc sp. 13PaB307
Nostoc sp. 1PaB307
Nostoc sp. 1
Nostoc sp. 2RC
Nostoc sp. 259
Nostoc sp. 34PaA307
Nostoc sp. 4PaM307
Nostoc sp. ACSSI 057
Nostoc sp. ARC10
Nostoc sp. ARC11
Nostoc sp. ARC12
Nostoc sp. ARC1
Nostoc sp. ARC2
Nostoc sp. ARC3
Nostoc sp. ARC4
Nostoc sp. ARC5
Nostoc sp. ARC6
Nostoc sp. ARC7
Nostoc sp. ARC8
Nostoc sp. ARC9
Nostoc sp. ATCC 43529
Nostoc sp. ATCC 53789
Nostoc sp. B(2019)
Nostoc sp. Baikal
Nostoc sp. C052
Nostoc sp. C057
Nostoc sp. CENA356
Nostoc sp. CENA543
Nostoc sp. CG10
Nostoc sp. CG11
Nostoc sp. CG2
Nostoc sp. CG6
Nostoc sp. CG8
Nostoc sp. CHAB 5715
Nostoc sp. CHAB 5824
Nostoc sp. CHAB 5836
Nostoc sp. CHAB 5844
Nostoc sp. CMAA1605
Nostoc sp. Exeter
Nostoc sp. FACHB-110
Nostoc sp. FACHB-133
Nostoc sp. FACHB-145
Nostoc sp. FACHB-152
Nostoc sp. FACHB-190
Nostoc sp. FACHB-280
Nostoc sp. FACHB-87
Nostoc sp. FACHB-888
Nostoc sp. FACHB-892
Nostoc sp. FACHB-973
Nostoc sp. FHMS
Nostoc sp. FUR 94210
Nostoc sp. G8
Nostoc sp. HK-01
Nostoc sp. Ind36
Nostoc sp. Ind37
Nostoc sp. Ind39
Nostoc sp. Ind40
Nostoc sp. KVJ20
Nostoc sp. KVJ3
Nostoc sp. LBG-1
Nostoc sp. LCRNK_10
Nostoc sp. LCRNK_11
Nostoc sp. LCRNK_12
Nostoc sp. LCRNK_16
Nostoc sp. LCRNK_17
Nostoc sp. LCRNK_1
Nostoc sp. LCRNK_24
Nostoc sp. LCRNK_25
Nostoc sp. LCRNK_29
Nostoc sp. LCRNK_30
Nostoc sp. LEGE 06077
Nostoc sp. LEGE 06106
Nostoc sp. LEGE 06150
Nostoc sp. LEGE 06158
Nostoc sp. LEGE 12447
Nostoc sp. LEGE 12450
Nostoc sp. MCT-1
Nostoc sp. MFG-1
Nostoc sp. MG11
Nostoc sp. MN-12A
Nostoc sp. MN-4A
Nostoc sp. MN-5A
Nostoc sp. MN-5B
Nostoc sp. MN-9A
Nostoc sp. MS1
Nostoc sp. N2
Nostoc sp. NDUPC003
Nostoc sp. NG2019
Nostoc sp. NIES-2111
Nostoc sp. NIES-3756
Nostoc sp. NIES-4103
Nostoc sp. NQAIF313
Nostoc sp. NOAIF314
Nostoc sp. NQAIF320
Nostoc sp. NZL
Nostoc sp. P1-15
Nostoc sp. P2-1
Nostoc sp. PA-18-2419
Nostoc sp. PCC 6720
Nostoc sp. PCC 7107
Nostoc sp. PCC 7120 = FACHB-418
Nostoc sp. PCC 7524
Nostoc sp. PTV
Nostoc sp. RF31YmG
Nostoc sp. RNMN
Nostoc sp. T09
Nostoc sp. TCL240-02
Nostoc sp. TCL26-01
Nostoc sp. UAM 362
Nostoc sp. UAM 367
Nostoc sp. UCD120
Nostoc sp. UCD122
Nostoc sp. UHCC 0702
Nostoc sp. UHCC 0870
Nostoc sp. UHCC 0926
Nostoc sp. UIC 10630
Nostoc sp. WHI
Nostoc sp. XA010
Nostoc sp. ruthii
Nostoc sp.
Nostoc sphaeroides CCNUC1
Nostoc sphaeroides
Nostoc spongiaeforme Ind41
Nostoc spongiaeforme Ind42
Nostoc spongiaeforme
Nostoc
Nostocaceae cyanobacterium CENA352
Nostocaceae cyanobacterium CENA373
Nostocaceae cyanobacterium CENA376
Nostocaceae cyanobacterium CENA379
Nostocaceae cyanobacterium CENA388
Nostocaceae
Nostocales cyanobacterium CCM-UFV026
Nostocales cyanobacterium CCM-UFV036
Nostocales cyanobacterium hetDA
Nostocales cyanobacterium
Nostocales
Nostochopsis sp. Ind28
Nothophytophthora sp. Chile5
Nothoprocta perdicaria
Novacetimonas hansenii
Noviherbaspirillum aerium
Noviherbaspirillum aridicola
Noviherbaspirillum cabarnae
Noviherbaspirillum galbum
Noviherbaspirillum humi
Noviherbaspirillum soli
Noviherbaspirillum sp. L7-7A
Noviherbaspirillum sp. UKPF54
Novimethylophilus kurashikiensis
Novipirellula artificiosorum
Novipirellula aureliae
Novispirillum itersonii
Novisyntrophococcus fermenticellae
Novosphingobium aquimarimum
Novosphingobium aromaticivorans DSN 12444
Novosphingobium aromaticivorans
Novosphingobium capsulatum
Novosphingobium chloroacetimidivorans
Novosphingobium decolorationis
Novosphingobium fuchskuhlense
Novosphingobium indicum
Novosphingobium lentum
Novosphingobium lindaniclasticum LE124
Novosphingobium lindaniclasticum
Novosphingobium malaysiense
Novosphingobium meiothermophilum
Novosphingobium naphthalenivorans
Novosphingobium nitrogenifigens DSM 19370
Novosphingobium nitrogenifigens
Novosphingobium pentaromativorans US6-1
Novosphingobium pentaromativorans
Novosphingobium profundi
Novosphingobium resinovorum
Novosphingobium rosa
Novosphingobium sp. 12-62-10
Novosphingobium sp. 17-62-9
Novosphingobium sp. 28-62-57
Novosphingobium sp. 32-60-15
Novosphingobium sp. Ap12
Novosphingobium sp. B-7
Novosphingobium sp. B1949
Novosphingobium sp. B1
Novosphingobium sp. B2580
Novosphingobium sp. B2638
Novosphingobium sp. BW1
Novosphingobium sp. CCH12-A3
Novosphingobium sp. CF614
Novosphingobium sp. FSW06-99
Novosphingobium sp. Fuku2-ISO-50
Novosphingobium sp. GeG2
Novosphingobium sp. HK4-1
Novosphingobium sp. KACC 22771
Novosphingobium sp. Leaf2
Novosphingobium sp. MBES04
Novosphingobium sp. P6W
Novosphingobium sp. PASSN1
Novosphingobium sp. PC22D
Novosphingobium sp. PP1Y
Novosphingobium sp. Rr 2-17
Novosphingobium sp. SG751A
Novosphingobium sp. ST904
Novosphingobium sp. THN1
Novosphingobium subterraneum
Novosphingobium
Novosphingopyxis baekryungensis
Novosphingopyxis iocasae
Novosphingopyxis sp. YJ-S2-01
Nunduva sp. LEGE 07159
OM182 bacterium BACL3 MAG-120507-bin80
OM182 bacterium BACL3 MAG-120619-bin3
OM182 bacterium BACL3 MAG-120920-bin41
Occallatibacter riparius
Oceanibaculum indicum P24
Oceanibaculum indicum
Oceanibaculum pacificum
Oceanicaulis sp. HTCC2633
Oceanicola sp. 22II-s10i
Oceanidesulfovibrio indonesiensis
Oceanidesulfovibrio marinus
Oceaniradius stylonematis
Oceanithermus profundus DSM 14977
Oceanobacillus halophilus
Oceanobacillus iheyensis HTE831
Oceanobacillus jeddahense
Oceanobacillus manasiensis
Oceanobacillus oncorhynchi
Oceanobacillus picturae
Oceanobacillus profundus
Oceanobacillus senegalensis
Oceanobacillus timonensis
Oceanobacillus
Oceanobacter kriegii
Oceanobacter mangrovi
Oceanobacter sp. RED65
Oceanobacter sp.
Oceanospirillum linum
Oceanospirillum sp. D5
Oceanotoga sp. DSM 15011
Oceanotoga teriensis
Ochrobactrum sp. PZHK4
Ochrobactrum sp.
Octadecobaeter argticus 238
Octadecobaeter argticus
Octadecobaeter ascidiaceicola
Octadecobaeter temperatus
Octodon degus
Oculatella sp. FACHB-28
Oculatella sp. LEGE 06141
Oerskovia douganii
Oerskovia enterophila
Oerskovia gallyi
Oerskovia jenensis
Oerskovia merdavium
Oerskovia paurometabola
Oerskovia rustica
Oerskovia sp. KBS0722
Oerskovia sp. Root918
Oerskovia turbata
Oharaeibacter diazotrophicus
Okeania comitata 3L-OSC
Okeania erythroflocculosa FFP12-1
Okeania hirsuta NAC11-70
Okeania hirusta
Okeania lorea FK12-17
Okeania plumata BCBC12-21
Okeania plumata FK12-27
Okeania plumata FK12-4
Okeania plumata NAB11-47
Okeania plumata NAC8-45
Okeania plumata NAC8-46
Okeania sp. A5
Okeania sp. KiyG1
Olea europaea var. sylvestris
Oleiagrimonas sp. C23AA
Oleiagrimonas sp. MCCC 1A03011
Oleidesulfovibrio alaskensis G20
Oleidesulfovibrio alaskensis
Oleiharenicola lentus
Oleiphilus sp. HI0086
Oleiphilus sp. HI0125
Oleisolibacter albus
Oleispira antarctica RB-8
Oleomonas cavernae
Olivibacter domesticus
Olivibacter sp. SDN3
Olivibacter
Olsenella massilliensis
Olsenella sp. HMSC062G07
Omnitrophica WOR_2 bacterium SM23_72
Omnitrophica bacterium OLB16
Opitutaceae bacterium EW11
Opitutaceae bacterium TAV1
Opitutaceae bacterium TAV3
Opitutaceae bacterium TAV4
Opitutaceae bacterium TAV5
Opitutaceae bacterium TSB47
Opitutae bacterium
Opitutales bacterium TMED158
Opitutus sp. GAS368
Opitutus sp. WL0086
Orenia marismortui
Orenia metallireducens
Oribacterium sp. C9
Oricola sp.
Ornatilinea apprima
Ornithinibacillus contaminans
Ornithinibacillus scapharcae
Ornithinicoccus hortensis
Ornithinimicrobium cerasi
Ornithinimicrobium sediminis
Ornithinimicrobium sp. HY1793
Orrella marina
Oryza sativa Indica Group
Oryza sativa Japonica Group
Oryza sativa
Oryzicola mucosus
Oryzisolibacter propanilivorax
Oryzomonas japonica
Oryzomonas rubra
Oryzomonas sagensis
Oryzomonas
Oscillatoria acuminata PCC 6304
Oscillatoria acuminata
Oscillatoria nigro-viridis PCC 7112
Oscillatoria nigro-viridis
Oscillatoria salina
Oscillatoria sancta AYJ113
Oscillatoria sancta PCC 7515
Oscillatoria sp. CCAP 1459/26
Oscillatoria sp. CS-180
Oscillatoria sp. FACHB-1406
Oscillatoria sp. FACHB-1407
Oscillatoria sp. HE19RPO
Oscillatoria sp. MMG-2
Oscillatoria sp. PCC 10802
Oscillibacter ruminantium
Oscillibacter sp. PC13
Oscillibacter valericigenes Sjm18-20
Oscillochloris sp. ZM17-4
Oscillochloris sp.
Oscillochloris trichoides DG-6
Oscillochloris trichoides
Oscillospiraceae bacterium CM
Oscillospiraceae bacterium
Oscillospiraceae
Osedax symbiont Rs1
Osedax symbiont Rs2
Ostreococcus tauri
Ottowia sp. GY511
Ottowia testudinis
Oxalicibacterium faecigallinarum
Oxalicibacterium flavum
Oxalobacter sp.
Oxobacter pfennigii
Oxynema aestuarii
Oxynema sp. CENA135
Pacificibacter maritimus
Pacificispira spongiicola
Paenarthrobacter histidinolovorans
Paenarthrobacter ilicis
Paenarthrobacter nitroguajacolicus
Paenarthrobacter sp. A20
Paenarthrobacter sp. AR 02
Paenarthrobacter sp. CM16
Paenarthrobacter sp. DKR-5
Paenarthrobacter sp. GOM3
Paenarthrobacter sp. JL.01a
Paenarthrobacter sp. MMS21-TAE1-1
Paenarthrobacter sp. PAE-2
Paenarthrobacter sp. UW852
Paenarthrobacter sp. YJN-5
Paenarthrobacter ureafaciens
Paenarthrobacter
Paenibacillus abekawaensis
Paenibacillus aceti
Paenibacillus agaridevorans
Paenibacillus albicereus
Paenibacillus albidus
Paenibacillus albiflavus
Paenibacillus alginolyticus
Paenibacillus algorifonticola
Paenibacillus alvei A6-6i-x
Paenibacillus alvei TS-15
Paenibacillus amylolyticus
Paenibacillus anaericanus
Paenibacillus antibioticophila
Paenibacillus apii
Paenibacillus apis
Paenibacillus barengoltzii
Paenibacillus beijingensis
Paenibacillus borealis
Paenibacillus bouchesdurhonensis
Paenibacillus bovis
Paenibacillus brasilensis
Paenibacillus camerounensis
Paenibacillus caui
Paenibacillus chitinolyticus
Paenibacillus curdlanolyticus YK9
Paenibacillus curdlanolyticus
Paenibacillus daejeonensis
Paenibacillus dakarensis
Paenibacillus darwinianus
Paenibacillus donghaensis
Paenibacillus durus ATCC 35681
Paenibacillus durus
Paenibacillus ehimensis
Paenibacillus elgii
Paenibacillus etheri
Paenibacillus faecis
Paenibacillus fonticola
Paenibacillus forsythiae
Paenibacillus fujiensis
Paenibacillus ginsengihumi
Paenibacillus gorillae
Paenibacillus graminis
Paenibacillus harenae
Paenibacillus helianthi
Paenibacillus herberti
Paenibacillus ihuae
Paenibacillus ihumii
Paenibacillus illinoisensis
Paenibacillus jamilae
Paenibacillus jilunlii
Paenibacillus kribbensis
Paenibacillus larvae subsp. pulvifaciens
Paenibacillus larvae
Paenibacillus lautus
Paenibacillus macerans
Paenibacillus massiliensis
Paenibacillus maysiensis
Paenibacillus monticola
Paenibacillus mucilaginosus 3016
Paenibacillus mucilaginosus K02
Paenibacillus mucilaginosus KNP414
Paenibacillus mucilaginosus
Paenibacillus odorifer
Paenibacillus oralis
Paenibacillus pabuli
Paenibacillus pasadenensis
Paenibacillus peoriae
Paenibacillus phocaensis
Paenibacillus physcomitrellae
Paenibacillus pini JCM 16418
Paenibacillus pini
Paenibacillus pinihumi
Paenibacillus pinisoli
Paenibacillus piscarius
Paenibacillus polymyxa ATCC 842
Paenibacillus polymyxa CR1
Paenibacillus polymyxa M1
Paenibacillus polymyxa SC2
Paenibacillus polymyxa
Paenibacillus prosopidis
Paenibacillus psychroresistens
Paenibacillus rhizophilus
Paenibacillus rhizosphaerae
Paenibacillus riograndensis SBR5
Paenibacillus riograndensis
Paenibacillus rubinfantis
Paenibacillus sabinae T27
Paenibacillus sabinae
Paenibacillus salinicaeni
Paenibacillus sanguinis
Paenibacillus senegalensis
Paenibacillus senegalimassiliensis
Paenibacillus silagei
Paenibacillus solani
Paenibacillus sonchi X19-5
Paenibacillus sonchi
Paenibacillus sophorae S27
Paenibacillus sophorae
Paenibacillus sp. 1-18
Paenibacillus sp. 1011MAR3C5
Paenibacillus sp. 19GGS1-52
Paenibacillus sp. 276b
Paenibacillus sp. 32O-W
Paenibacillus sp. 453mf
Paenibacillus sp. 7516
Paenibacillus sp. 7523-1
Paenibacillus sp. A3
Paenibacillus sp. A78
Paenibacillus sp. AR247
Paenibacillus sp. AS2(2010)
Paenibacillus sp. Aloe-11
Paenibacillus sp. B01
Paenibacillus sp. BGI2013
Paenibacillus sp. BJ-4
Paenibacillus sp. BY55
Paenibacillus sp. Bb24
Paenibacillus sp. Bb54
Paenibacillus sp. Bd43
Paenibacillus sp. Bs57
Paenibacillus sp. By56
Paenibacillus sp. CAA11
Paenibacillus sp. CF095
Paenibacillus sp. CGMCC 1.18879
Paenibacillus sp. CQ-X
Paenibacillus sp. D9
Paenibacillus sp. DF4MM9
Paenibacillus sp. DMB20
Paenibacillus sp. DMB5
Paenibacillus sp. E194
Paenibacillus sp. EKM205P
Paenibacillus sp. EKM206P
Paenibacillus sp. EKM212P
Paenibacillus sp. FJAT-27812
Paenibacillus sp. FSL H7-0326
Paenibacillus sp. FSL H7-0357
Paenibacillus sp. FSL H7-0737
Paenibacillus sp. FSL H7-689
Paenibacillus sp. FSL H8-237
Paenibacillus sp. FSL H8-457
Paenibacillus sp. FSL P4-0081
Paenibacillus sp. FSL R5-0765
Paenibacillus sp. FSL R5-0912
Paenibacillus sp. FSL R5-192
Paenibacillus sp. FSL R7-0273
Paenibacillus sp. FSL R7-0331
Paenibacillus sp. FSL R7-269
Paenibacillus sp. FSL R7-277
Paenibacillus sp. G11
Paenibacillus sp. GA12
Paenibacillus sp. GA5
Paenibacillus sp. GJ10
Paenibacillus sp. GJ11
Paenibacillus sp. GJ24
Paenibacillus sp. GJ46
Paenibacillus sp. GJ52
Paenibacillus sp. GJ9
Paenibacillus sp. GM1FR
Paenibacillus sp. GZGTW-1
Paenibacillus sp. Gc58
Paenibacillus sp. HB-43
Paenibacillus sp. HGF5
Paenibacillus sp. HMSSN-139
Paenibacillus sp. HW 567
Paenibacillus sp. Hp7
Paenibacillus sp. IHB B 3415
Paenibacillus sp. IHBB 10380
Paenibacillus sp. JCM 10914
Paenibacillus sp. JDR-2
Paenibacillus sp. JMULE4
Paenibacillus sp. JT91
Paenibacillus sp. LK1
Paenibacillus sp. Leaf72
Paenibacillus sp. MAEPY1
Paenibacillus sp. MAEPY2
Paenibacillus sp. MYb63
Paenibacillus sp. MYb67
Paenibacillus sp. MZ03-122A
Paenibacillus sp. N3.4
Paenibacillus sp. NFR01
Paenibacillus sp. NH-1
Paenibacillus sp. Nz28
Paenibacillus sp. Nz29
Paenibacillus sp. Nz30
Paenibacillus sp. OK060
Paenibacillus sp. OK076
Paenibacillus sp. OSY-SE
Paenibacillus sp. P1XP2
Paenibacillus sp. P22
Paenibacillus sp. P32E
Paenibacillus sp. P3E
Paenibacillus sp. P46E
Paenibacillus sp. PCH8
Paenibacillus sp. PDC88
Paenibacillus sp. PK3_47
Paenibacillus sp. R-45815
Paenibacillus sp. R-46252
Paenibacillus sp. R-46257
Paenibacillus sp. R-46269
Paenibacillus sp. R1-312
Paenibacillus sp. RU26A
Paenibacillus sp. RU5A
Paenibacillus sp. RU5M
Paenibacillus sp. Root444D2
Paenibacillus sp. Root52
Paenibacillus sp. S150
Paenibacillus sp. Sl51
Paenibacillus sp. Soil522
Paenibacillus sp. Soil724D2
Paenibacillus sp. Soil750
Paenibacillus sp. Soil766
Paenibacillus sp. Soil787
Paenibacillus sp. Ss35
Paenibacillus sp. Sx52
Paenibacillus sp. TCA20
Paenibacillus sp. TD94
Paenibacillus sp. UNC217MF
Paenibacillus sp. UNC451MF
Paenibacillus sp. URB8-2
Paenibacillus sp. VMFN-D1
Paenibacillus sp. VT-400
Paenibacillus sp. XY044
Paenibacillus sp. Y412MC10
Paenibacillus sp. YN15
Paenibacillus sp. cl130
Paenibacillus sp. g2
Paenibacillus sp. lzh-N1
Paenibacillus sp. oral taxon 786 str. D14
Paenibacillus sp. oral taxon 786
Paenibacillus sp. p3-SID1389
Paenibacillus spiritus
Paenibacillus stellifer
Paenibacillus taichungensis
Paenibacillus taiwanensis
Paenibacillus taohuashanense
Paenibacillus tengchongensis
Paenibacillus tepidiphilus
Paenibacillus terrae HPL-003
Paenibacillus terrae
Paenibacillus terrigena
Paenibacillus thiaminolyticus
Paenibacillus tianjinensis
Paenibacillus tianmuensis
Paenibacillus timonensis
Paenibacillus tritici
Paenibacillus triticisoli
Paenibacillus typhae
Paenibacillus tyrfis
Paenibacillus vortex V453
Paenibacillus vortex
Paenibacillus wenxiniae
Paenibacillus wulumugiensis
Paenibacillus wynnii
Paenibacillus xylanexedens
Paenibacillus xylanivorans
Paenibacillus yonginensis
Paenibacillus zanthoxyli
Paenibacillus
Paeniclostridium sordellii 8483
Paeniclostridium sordellii
Paeniglutamicibacter cryotolerans
Paeniglutamicibacter kerguelensis
Paeniglutamicibacter quisquiliarum
Paeniglutamicibacter sp. Y32M11
Paeniglutamicibacter sp. ZC-3
Paeniglutamicibacter terrestris
Paenihalocynthiibacter styelae
Paenisporosarcina cavernae
Paenisporosarcina sp. HGH0030
Paenisporosarcina sp. TG-14
Palleronia abyssalis
Palleronia marisminoris
Paludibacter jiangxiensis
Paludibacter propionicigenes WB4
Paludibacter propionicigenes
Paludibacterium yongneupense
Pan troglodytes
Pandoraea captiosa
Pandoraea thiooxydans
Panicum miliaceum
Pannonibacter carbonis
Pannonibacter phragmitetus
Pannonibacter sp. SL95
Pantoea agglomerans
Pantoea ananatis
Pantoea cypripedii
Pantoea dispersa
Pantoea sp. A0305
Pantoea sp. A0310
Pantoea sp. AS-PWVM4
Pantoea sp. Ap-967
Pantoea sp. At-9b
Pantoea sp. CCBAU 10857
Pantoea sp. CCBAU 10904
Pantoea sp. CRLI0712a
Pantoea sp. CRLI0721a
Pantoea sp. CRLI0724b
Pantoea sp. CRLI0725a
Pantoea sp. CRLS0621
Pantoea sp. CRLS062b
Pantoea sp. CRRI-88_WR23
Pantoea sp. EC080527_02
Pantoea sp. MSR2
Pantoea sp. P0352
Pantoea sp. P0356
Pantoea sp. P0359
Pantoea sp. R-46239
Pantoea sp. SB19
Pantoea sp. Taur
Pantoea sp. Tr-811
Pantoea sp. gx-125
Pantoea sp.
Pantoea stewartii subsp. stewartii DC283
Pantoea stewartii
Papiliotrema laurentii
Papillibacter cinnamivorans DSM 12816
Parabacteroides distasonis
Parabacteroides faecis
Parabacteroides merdae
Parabacteroides sp. FAFU027
Parabacteroides sp. Marseille-P3160
Paraburkholderia acidisoli
Paraburkholderia aromaticivorans
Paraburkholderia aspalathi
Paraburkholderia atlantica
Paraburkholderia bannensis
Paraburkholderia bryophila
Paraburkholderia caballeronis
Paraburkholderia caledonica
Paraburkholderia caribensis
Paraburkholderia diazotrophica
Paraburkholderia dilworthii
Paraburkholderia dioscoreae
Paraburkholderia dipogonis
Paraburkholderia ferrariae
Paraburkholderia franconis
Paraburkholderia fungorum
Paraburkholderia guartelaensis
Paraburkholderia hospita
Paraburkholderia kirstenboschensis
Paraburkholderia kururiensis
Paraburkholderia lycopersici
Paraburkholderia madseniana
Paraburkholderia metrosideri
Paraburkholderia mimosarum
Paraburkholderia nodosa
Paraburkholderia phenoliruptrix BR3459a
Paraburkholderia phenoliruptrix
Paraburkholderia phosphatilytica
Paraburkholderia phymatum STM815
Paraburkholderia phymatum
Paraburkholderia phytofirmans
Paraburkholderia piptadeniae
Paraburkholderia podalyriae
Paraburkholderia rhynchosiae
Paraburkholderia ribeironis
Paraburkholderia sabiae
Paraburkholderia sartisoli
Paraburkholderia silvatlantica SRMrh-20
Paraburkholderia silvatlantica
Paraburkholderia sp. Ac-20340
Paraburkholderia sp. Ac-20342
Paraburkholderia sp. Ac-20347
Paraburkholderia sp. BCC1876
Paraburkholderia sp. BCC1886
Paraburkholderia sp. BL10I2N1
Paraburkholderia sp. BL17N1
Paraburkholderia sp. BL18I3N2
Paraburkholderia sp. BL21I4N1
Paraburkholderia sp. BL23IIN1
Paraburkholderia sp. BL25IIN1
Paraburkholderia sp. BL2714N3
Paraburkholderia sp. BL6665CI2N2
Paraburkholderia sp. BL6669N2
Paraburkholderia sp. BL8N3
Paraburkholderia sp. BL9I2N2
Paraburkholderia sp. C35
Paraburkholderia sp. CNPSo 3076
Paraburkholderia sp. CNPSo 3272
Paraburkholderia sp. Cpub6
Paraburkholderia sp. DHOC27
Paraburkholderia sp. HC6.4b
Paraburkholderia sp. JPY419
Paraburkholderia sp. Kb1A
Paraburkholderia sp. MM5384-R2
Paraburkholderia sp. RAU2J
Paraburkholderia sp. SOS3
Paraburkholderia sp. Se-20369
Paraburkholderia sp. UCT2
Paraburkholderia sp. UCT31
Paraburkholderia sp. UYCP14C
Paraburkholderia sp.
Paraburkholderia sprentiae WSM5005
Paraburkholderia sprentiae
Paraburkholderia steynii
Paraburkholderia strydomiana
Paraburkholderia terrae
Paraburkholderia terricola
Paraburkholderia tropica
Paraburkholderia tuberum STM678
Paraburkholderia tuberum
Paraburkholderia ultramafica
Paraburkholderia unamae MTI-641
Paraburkholderia unamae
Paraburkholderia xenovorans LB400
Paraburkholderia xenovorans
Paraburkholderia youngii
Paraburkholderia
Parachryseolinea silvisoli
Paraclostridium benzoelyticum
Paraclostridium bifermentans subsp. muricolitidis
Paraclostridium bifermentans
Paraclostridium dentum
Paracnuella aquatica
Paracoccaceae bacterium
Paracoccaceae
Paracoccus aeridis
Paracoccus aestuariivivens
Paracoccus alkanivorans
Paracoccus alkenifer
Paracoccus aminovorans
Paracoccus chinensis
Paracoccus hibiscisoli
Paracoccus liaowangingii
Paracoccus lichenicola
Paracoccus litorisediminis
Paracoccus marcusii
Paracoccus marinus
Paracoccus onubensis
Paracoccus pantotrophus
Paracoccus saliphilus
Paracoccus sediminis
Paracoccus shandongensis
Paracoccus sp. 08
Paracoccus sp. 228
Paracoccus sp. CCBAU 10852
Paracoccus sp. CCBAU 10893
Paracoccus sp. NBH48
Paracoccus sp. PAMC 22219
Paracoccus sp. R-46302
Paracoccus sp. S-4012
Paracoccus sp. S4493
Paracoccus sp. SM22M-07
Paracoccus sp. TK19116
Paracoccus sp. Z118
Paracoccus suum
Paracoccus
Paraconexibacter algicola
Paraconexibacter antarcticus
Paracraurococcus ruber
Paracraurococcus sp. NE82
Paradesulfitobacterium ferrireducens
Paraeggerthella hongkongensis
Paraeggerthella sp. Marseille-Q4926
Paraeggerthella
Parageobacillus galactosidasius
Parageobacillus genomosp. 1
Parageobacillus thermantarcticus
Parageobacillus thermoglucosidasius C56-YS93
Parageobacillus thermoglucosidasius NBRC 107763
Parageobacillus thermoglucosidasius TNO-09.020
Parageobacillus thermoglucosidasius
Paraglaciecola agarilytica NO2
Paraglaciecola arctica BSs20135
Paraglaciecola arctica
Paraglaciecola chathamensis S18K6
Paraglaciecola hydrolytica
Paraglaciecola polaris LMG 21857
Paraglaciecola polaris
Paraglaciecola psychrophila 170
Paraglaciecola psychrophila
Parahaliea maris
Paraliomyxa miuraensis
Paramaledivibacter caminithermalis DSM 15212
Paramaledivibacter caminithermalis
Paraneptunicella aestuarii
Paraoerskovia marina
Parapedobacter pyrenivorans
Pararcticibacter amylolyticus
Pararheinheimera mesophila
Pararheinheimera texasensis
Pararhizobium arenae
Pararhodospirillum oryzae
Pararhodospirillum photometricum DSM 122
Pararhodospirillum photometricum
Pararobbsia alpina
Pararobbsia silviterrae
Parasegetibacter sp. NRK P23
Parasporobacterium paucivorans DSM 15970
Parasporobacterium paucivorans
Parasulfuritortus cantonensis
Parazoarcus communis
Parcubacteria bacterium 32_520
Parcubacteria bacterium DG_72
Parcubacteria bacterium DG_74_1
Parcubacteria bacterium DG_74_2
Parcubacteria bacterium DG_74_3
Parvularcula bermudensis
Parvularcula oceani
Patulibacter americanus
Patulibacter medicamentivorans
Patulibacter minatonensis
Patulibacter sp. SYSU D01012
Paucibacter sp. B51
Paucibacter sp. KCTC 42545
Paucibacter sp. M5-1
Paucibacter sp. 01-1
Paucibacter sp. PLA-PC-4
Paucibacter sp. XJ19-41
Paucibacter toxinivorans
Paucidesulfovibrio gracilis DSM 16080
Paucidesulfovibrio gracilis
Paucidesulfovibrio longus
Paucimonas lemoignei
Paucisalibacillus globulus
Paucisalibacillus sp. EB02
Pectinatus brassicae
Pectinatus cerevisiiphilus
Pectinatus frisingensis
Pectinatus haikarae
Pectinatus sottacetonis
Pectobacterium atrosepticum ICMP 1526
Pectobacterium atrosepticum SCRI1043
Pectobacterium atrosepticum
Pectobacterium betavasculorum
Pectobacterium cacticida
Pectobacterium carotovorum subsp. carotovorum
Pectobacterium carotovorum
Pectobacterium parmentieri WPP163
Pectobacterium parmentieri
Pectobacterium peruviense
Pectobacterium polaris
Pectobacterium sp.
Pectobacterium versatile
Pectobacterium
Pediococcus pentosaceus
Pedobacter africanus
Pedobacter agri
Pedobacter cryoconitis
Pedobacter duraquae
Pedobacter hartonius
Pedobacter heparinus
Pedobacter jeongneungensis
Pedobacter kyungheensis
Pedobacter miscanthi
Pedobacter mongoliensis
Pedobacter psychrotolerans
Pedobacter rhizosphaerae
Pedobacter roseus
Pedobacter sandarakinus
Pedobacter segetis
Pedobacter sp. AJM
Pedobacter sp. AK017
Pedobacter sp. ASV19
Pedobacter sp. BAL39
Pedobacter sp. BMA
Pedobacter sp. Bi27
Pedobacter sp. G11
Pedobacter sp. GR22-10
Pedobacter sp. HMF7056
Pedobacter sp. HMF7647
Pedobacter sp. HMWF019
Pedobacter sp. L105
Pedobacter sp. Leaf216
Pedobacter sp. Leaf250
Pedobacter sp. Leaf41
Pedobacter sp. MC2016-05
Pedobacter sp. MC2016-14
Pedobacter sp. MC2016-15
Pedobacter sp. MC2016-24
Pedobacter sp. MR2016-24
Pedobacter sp. MR22-3
Pedobacter sp. R20-19
Pedobacter sp. SYSU D00382
Pedobacter sp. SYSU D00535
Pedobacter sp. SYSU D00823
Pedobacter sp. SYSU D00873
Pedobacter westerhofensis
Pedobacter xixiisoli
Pedococcus badiiscoriae
Pelagibacterium sediminicola
Pelagibaculum spongiae
Pelagicoccus albus
Pelagicola sp. LXJ1103
Pelagimonas varians
Pelagivirga dicentrarchi
Pelobacter propionicus DSM 2379
Pelobacter propionicus
Pelobacter seleniigenes
Pelobium manganitolerans
Pelodictyon luteolum DSM 273
Pelodictyon luteolum
Pelodictyon phaeoclathratiforme BU-1
Pelodictyon phaeoclathratiforme
Pelolinea submarina
Pelomicrobium methylotrophicum
Pelomonas puraquae
Pelomonas saccharophila
Pelomonas sp. KK5
Pelomonas sp. P7
Pelomonas sp. P8
Pelomonas sp. Root1217
Pelomonas sp. Root1237
Pelomonas sp. Root1444
Pelomonas sp. UHG3
Pelorhabdus rhamnosifermentans
Pelosinus fermentans A11
Pelosinus fermentans B4
Pelosinus fermentans DSM 17108
Pelosinus fermentans JBW45
Pelosinus fermentans
Pelosinus propionicus DSM 13327
Pelosinus propionicus
Pelosinus sp. Bkl1
Pelosinus sp. UFO1
Pelosinus
Pelotomaculum propionicicum
Pelotomaculum schinkii
Pelotomaculum sp. FP
Pelotomaculum sp. PtaB.Bin013
Pelotomaculum sp. PtaB.Bin104
Pelotomaculum sp. PtaB.Bin117
Pelotomaculum sp. PtaU1.Bin035
Pelotomaculum sp. PtaU1.Bin065
Pelotomaculum terephthalicicum
Pelotomaculum thermopropionicum SI
Pelotomaculum thermopropionicum
Pelotomaculum
Pelovirga terrestris
Penaeus vannamei
Pengzhenrongella sicca
Peptoanaerobacter stomatis
Peptoclostridium acidaminophilum DSM 3953
Peptoclostridium acidaminophilum
Peptoclostridium litorale DSM 5388
Peptoclostridium litorale
Peptococcaceae bacterium 1109
Peptococcaceae bacterium BICA1-7
Peptococcaceae bacterium BICA1-8
Peptococcaceae bacterium BRH_c23
Peptococcaceae bacterium BRH_c4a
Peptococcaceae bacterium BRH_c4b
Peptococcaceae bacterium BRH_c8a
Peptococcaceae bacterium CEB3
Peptococcaceae bacterium SCADC1_2_3
Peptococcaceae bacterium
Peptococcus niger
Peptoniphilus sp. oral taxon 375 str. F0436
Peptoniphilus urinimassiliensis
Peptostreptococcaceae bacterium AS15
Peptostreptococcaceae bacterium pGA-8
Peribacillus butanolivorans
Peribacillus deserti
Peribacillus frigoritolerans
Peribacillus kribbensis
Peribacillus loiseleuriae
Peribacillus muralis
Peribacillus simplex
Peribacillus sp. TH16
Perlucidibaca piscinae
Persephonella atlantica
Persephonella hydrogeniphila
Persephonella marina EX-H1
Persephonella marina
Persephonella sp. KM09-Lau-8
Persicimonas caeni
Persicitalea jodogahamensis
Peterkaempfera bronchialis
Peterkaempfera griseoplana
Peteryoungia ipomoeae
Petrocella atlantisensis
Petroclostridium xylanilyticum
Petrotoga halophila DSM 16923
Petrotoga halophila
Petrotoga mexicana DSM 14811
Petrotoga mexicana
Petrotoga miotherma DSM 10691
Petrotoga mobilis
Petrotoga sp. 9T1HF07.CasAA.8.2
Petrotoga
Phaeacidiphilus oryzae
Phaeobacter italicus
Phaeobacter sp. 22II1-1F12B
Phaeobacter sp. CECT 5382
Phaeocystidibacter marisrubri
Phaeovulum veldkampii DSM 11550
Phaeovulum veldkampii
Phaeovulum vinaykumarii
Phascolarctobacterium faecium DSM 14760
Phascolarctobacterium faecium
Phascolarctobacterium sp. Marseille-Q4147
Phascolarctobacterium sp.
Phascolarctobacterium succinatutens CAG: 287
Phascolarctobacterium succinatutens YIT 12067
Phascolarctobacterium succinatutens
Phaseolus vulgaris
Phenylobacterium aquaticum
Phenylobacterium sp. J367
Phenylobacterium zucineum
Phocaeicola vulgatus
Phormidesmis priestleyi Ana
Phormidesmis priestleyi
Phormidium ambiguum
Phormidium pseudopriestleyi
Phormidium sp. AD1
Phormidium sp. FACHB-1136
Phormidium sp. FACHB-592
Phormidium sp. GEM2.Bin31
Phormidium sp. LEGE 05292
Phormidium sp. OSCR
Phormidium sp. PBR-2020
Phormidium sp. SCSIO X
Phormidium sp. SL48-SHIP
Phormidium tenue
Phormidium willei BDU 130791
Phormidium yuhuli AB48
Phormidium yuhuli
Photobacterium aphoticum
Photobacterium aquae
Photobacterium gaetbulicola Gung47
Photobacterium gaetbulicola
Photobacterium galatheae
Photobacterium halotolerans
Photobacterium lutimaris
Photobacterium marinum
Photobacterium phosphoreum
Photobacterium profundum SS9
Photobacterium profundum
Photobacterium sanctipauli
Photobacterium sanguinicancri
Photobacterium sp. SKA34
Photobacterium sp. WH24
Photobacterium sp. ZSDE20
Photobacterium toruni
Photobacterium
Phototrophicus methaneseepsis
Phreatobacter oligotrophus
Phycicoccus duodecadis
Phycicoccus elongatus Lp2
Phycicoccus flavus
Phycicoccus jejuensis
Phycicoccus mangrovi
Phycicoccus sp. BSK3Z-2
Phycicoccus sp. DTK01
Phycicoccus sp. HDW14
Phycicoccus sp. MQZ13P-5
Phycisphaera sp.
Phycisphaerae bacterium SG8_4
Phycisphaerae bacterium SM23_30
Phyllobacteriaceae
Phyllobacterium sophorae
Phyllobacterium sp. BIHB 4155
Phyllobacterium sp. BIHB 4156
Phyllobacterium sp. IY22
Phyllobacterium sp. ORS 1402
Phyllobacterium sp. ORS 1403
Phyllobacterium sp. YR531
Phyllobacterium zundukense
Physcomitrium patens
Phytoactinopolyspora halotolerans
Phytobacter diazotrophicus
Phytobacter palmae
Phytobacter sp. SCO41
Phytobacter ursingii
Phytohabitans flavus
Phytophthora kernoviae
Phytophthora megakarya
Phytophthora parasitica CJ01A1
Phytophthora parasitica P10297
Phytophthora parasitica P1569
Phytophthora parasitica P1976
Phytophthora parasitica
Phytophthora sojae
Pikeienuella piscinae
Pimelobacter simplex
Pimelobacter sp. 30-1
Pinibacter aurantiacus
Pinisolibacter aquiterrae
Pirellula staleyi
Pirellulaceae
Piscicoccus intestinalis
Piscinibacter defluvii
Piscirickettsia salmonis LF-89 = ATCC VR-1361
Piscirickettsia salmonis
Planctobacterium marinum
Planctomyces sp. SH-PL14
Planctomycetaceae bacterium
Planctomycetales bacterium ZRK34
Planctomycetales bacterium
Planctomycetes bacterium CA13
Planctomycetes bacterium DG_20
Planctomycetes bacterium DG_23
Planctomycetes bacterium DG_58
Planctomycetes bacterium SM23_25
Planctomycetes bacterium SM23_32
Planctomycetes bacterium TMED75
Planctomycetota bacterium
Planktomarina sp.
Planktotalea frisia
Planktothricoides sp. SR001
Planktothrix agardhii
Planktothrix mougeotii
Planktothrix paucivesiculata
Planktothrix pseudagardhii
Planktothrix rubescens
Planktothrix serta PCC 8927
Planktothrix serta
Planktothrix sp. FACHB-1355
Planktothrix sp. FACHB-1365
Planktothrix sp. FACHB-1375
Planktothrix sp. PCC 11201
Planktothrix tepida PCC 9214
Planktothrix tepida
Planktothrix
Planobispora longispora
Planobispora siamensis
Planobispora takensis
Planococcus antarcticus DSM 14505
Planococcus antarcticus
Planococcus donghaensis MPA1U2
Planococcus donghaensis
Planococcus halocryophilus Or1
Planococcus halocryophilus
Planococcus maritimus
Planococcus massiliensis
Planococcus plakortidis
Planococcus rifietoensis
Planococcus sp. CAU13
Planococcus sp. ISL-109
Planococcus sp. PAMC 21323
Planococcus wigleyi
Planomicrobium soli
Planomicrobium sp. MB-3u-38
Planomicrobium sp. REN14
Planomonospora parontospora
Planomonospora sp. ID67723
Planotetraspora mira
Planotetraspora silvatica
Planotetraspora sp. A-T 1434
Plantibacter cousiniae
Plantibacter flavus
Plantibacter sp. CFBP 8798
Plantibacter sp. Leaf314
Plantibacter sp. M259
Plantibacter sp. MCCC 1A11337
Plantibacter sp. PA-3-X8
Plantibacter sp. T3
Plantibacter sp. VKM Ac-1784
Plantibacter sp. VKM Ac-2876
Plantibacter sp. VKM Ac-2880
Plantibacter
Plasmid RK2
Plasmodium falciparum 3D7
Plasmopara halstedii
Plasticicumulans acidivorans
Plectonema cf. radiosum LEGE 06105
Plectonema radiosum
Pleomorphomonas carboxyditropha
Pleomorphomonas diazotrophica
Pleomorphomonas koreensis
Pleomorphomonas oryzae
Pleomorphomonas sp. COJ-58
Pleomorphomonas sp. JP5
Pleomorphomonas sp. NRK KF1
Pleomorphomonas sp. SM30
Plesiocystis pacifica
Plesiomonas shigelloides
Plesiomonas
Pleurocapsa sp. CCALA 161
Pleurocapsa sp. PCC 7319
Pleurocapsa sp. PCC 7327
Pleurocapsales
Pluralibacter gergoviae
Pocillopora damicornis
Polaribacter sp. BAL334
Polaribacter sp.
Polaromonas glacialis
Polaromonas naphthalenivorans CJ2
Polaromonas naphthalenivorans
Polaromonas sp. 24-62-144
Polaromonas sp. 28-63-22
Polaromonas sp. 35-63-240
Polaromonas sp. 39-63-203
Polaromonas sp. JS666
Polaromonas sp. OV174
Polyangiaceae bacterium UTPRO1
Polyangium aurulentum
Polyangium fumosum
Polyangium jinanense
Polyangium sp. rjm3
Polyangium spumosum
Polycyclovorans algicola
Polynucleobacter acidiphobus
Polynucleobacter aenigmaticus
Polynucleobacter alcilacus
Polynucleobacter antarcticus
Polynucleobacter campilacus
Polynucleobacter cosmopolitanus
Polynucleobacter difficilis
Polynucleobacter hirudinilacicola
Polynucleobacter kasalickyi
Polynucleobacter meluiroseus
Polynucleobacter necessarius
Polynucleobacter paneuropaeus
Polynucleobacter rarus
Polynucleobacter sinensis
Polynucleobacter sp. 24-46-87
Polynucleobacter sp. 32-46-5
Polynucleobacter sp. 35-46-11
Polynucleobacter sp. 71A-WALBACH
Polynucleobacter sp. AP-Titi-500A-B4
Polynucleobacter sp. MWH-Braz-FAM2G
Polynucleobacter sp. MWH-Svant-W18
Polynucleobacter sp. UB-Tiil-W10
Polynucleobacter sp. VK25
Polynucleobacter
Pomacea canaliculata
Pongo abelii
Pontibacillus chungwhensis BH030062
Pontibacillus chungwhensis
Pontibacillus halophilus JSM 076056 = DSM 19796
Pontibacillus halophilus
Pontibacillus litoralis JSM 072002
Pontibacillus litoralis
Pontibacillus marinus BH030004 = DSM 16465
Pontibacillus marinus
Pontibacillus yanchengensis Y32
Pontibacillus yanchengensis
Pontibacter actiniarum
Pontibacter akesuensis
Pontibacter amylolyticus
Pontibacter aquaedesilientis
Pontibacter arcticus
Pontibacter aydingkolensis
Pontibacter beigongshangensis
Pontibacter burrus
Pontibacter cellulosilyticus
Pontibacter chinhatensis
Pontibacter diazotrophicus
Pontibacter fetidus
Pontibacter flavimaris
Pontibacter indicus
Pontibacter mangrovi
Pontibacter mucosus
Pontibacter oryzae
Pontibacter pamirensis
Pontibacter pudoricolor
Pontibacter ramchanderi
Pontibacter roseus
Pontibacter ruber
Pontibacter russatus
Pontibacter silvestris
Pontibacter sp. E15-1
Pontibacter sp. FD36
Pontibacter sp. GY10130
Pontibacter sp. HSC-14F20
Pontibacter sp. HSC-36F09
Pontibacter sp. M82
Pontibacter sp. NBU2971
Pontibacter sp. NBU2972
Pontibacter sp. SGAir0037
Pontibacter sp. Tf4
Pontibacter sp. XAAS-72
Pontibacter ummariensis
Pontibacter virosus
Pontibacter
Pontiella desulfatans
Pontiella sulfatireligans
Pontivivens insulae
Populus euphratica
Porphyrobacter sp. CACIAM 03H1
Porphyrobacter sp. HT-58-2
Porphyrobacter sp. IPPAS B-1204
Porphyrobacter sp. LM 6
Porphyrobacter sp. TH134
Porphyromonadaceae bacterium
Porphyromonas gingivalis
Porphyromonas sp. oral taxon 278 str. W7784
Porphyromonas sp. oral taxon 278
Porticoccus hydrocarbonoclasticus
Poseidonibacter lekithochrous
Poseidonibacter parvus
Poseidonibacter sp. SJOD-M-33
Poseidonocella pacifica
Poseidonocella sp. HB161398
Posidoniimonas corsicana
Potamosiphon australiensis FHC0914.01
Potamosiphon australiensis FHC0914.02
Potamosiphon australiensis FHC0914.03
Prauserella aidingensis
Prauserella alba
Prauserella cavernicola
Prauserella flavalba
Prauserella halophila
Prauserella isguenensis
Prauserella marina
Prauserella rugosa
Prauserella salsuginis group
Prauserella sediminis
Prauserella shujinwangii
Prauserella sp. PE36
Prauserella
Prevotella amnii
Prevotella bivia
Prevotella bryantii B14
Prevotella bryantii
Prevotella cerevisiae
Prevotella corporis
Prevotella oryzae
Prevotella sp. CAG: 386
Prevotella sp. DNF00663
Prevotella sp. P6B1
Prevotella sp. P6B4
Priestia aryabhattai B8W22
Priestia aryabhattai
Priestia endophytica
Priestia flexa
Priestia koreensis
Priestia megaterium DSM 319
Priestia megaterium WSH-002
Priestia megaterium
Priestia
Primorskyibacter flagellatus
Primorskyibacter marinus
Primorskyibacter sedentarius
Primorskyibacter sp. XY-301
Prochlorococcus marinus str. AS9601
Prochlorococcus marinus str. MIT 1312
Prochlorococcus marinus str. MIT 1313
Prochlorococcus marinus str. MIT 1318
Prochlorococcus marinus str. MIT 1320
Prochlorococcus marinus str. MIT 1323
Prochlorococcus marinus str. MIT 1327
Prochlorococcus marinus str. MIT 1342
Prochlorococcus marinus str. MIT 9211
Prochlorococcus marinus str. MIT 9215
Prochlorococcus marinus str. MIT 9301
Prochlorococcus marinus str. MIT 9303
Prochlorococcus marinus str. MIT 9515
Prochlorococcus marinus str. NATL1A
Prochlorococcus sp. MIT 1303
Prochlorococcus sp. MIT 1306
Prochlorothrix hollandica
Profundibacterium mesophilum
Prolixibacter bellariivorans
Prolixibacter denitrificans
Prolixibacter sp. NT017
Prolixibacter sp. SD074
Promicromonospora citrea
Promicromonospora kroppenstedtii
Promicromonospora panici
Promicromonospora soli
Promicromonospora sukumoe
Promicromonospora umidemergens
Propionibacterium australiense
Propionibacterium cyclohexanicum
Propionibacterium freudenreichii
Propionibacterium ruminifibrarum
Propioniferax innocua
Propionispira arboris
Propionispira raffinosivorans
Propionispira
Propionispora hippei DSM 15287
Propionispora hippei
Propionispora sp. 2/2-37
Propionispora vibrioides
Propionispora
Propionivibrio dicarboxylicus
Propionivibrio limicola
Propionivibrio sp. SG131
Prosthecobacter debontii
Prosthecobacter dejongeii
Prosthecobacter fusiformis
Prosthecochloris aestuarii DSM 271
Prosthecochloris marina
Prosthecochloris sp. CIB 2401
Prosthecochloris sp. GSB1
Prosthecochloris sp. SCSIO W1101
Prosthecochloris sp. SCSIO W1103
Prosthecochloris sp. ZM
Prosthecochloris sp. ZM_2
Prosthecochloris vibrioformis
Prosthecochloris
Prosthecomicrobium hirschii
Proteiniborus ethanoligenes
Proteiniborus sp. DW1
Proteiniclasticum ruminis
Proteiniphilum saccharofermentans
Proteobacteria bacterium CAG: 495
Proteobacteria bacterium CG1_02_64_396
Proteobacteria bacterium HN_bin10
Proteobacteria bacterium SG_bin5
Proteobacteria bacterium SG_bin6
Proteobacteria bacterium SG_bin9
Proteobacteria bacterium ST_bin13
Proteocatella sphenisci
Proteus mirabilis
Proteus sp. HMSC10D02
Provencibacterium massiliense
Providencia sp. wls1919
Providencia stuartii
Prunus avium
Prunus mume
Prunus persica
Prunus serrulata
Prunus yedoensis var. nudiflora
Pseudacidobacterium ailaaui
Pseudacidovorax intermedius NH-1
Pseudacidovorax intermedius
Pseudacidovorax sp. NFM-22
Pseudacidovorax sp. NH-1
Pseudacidovorax sp. RU35E
Pseudacidovorax sp. ptl-2
Pseudanabaena biceps PCC 7429
Pseudanabaena biceps
Pseudanabaena cf. frigida LEGE 06144
Pseudanabaena cf. persicina LEGE 07163
Pseudanabaena sp. ‘Roaring Creek’
Pseudanabaena sp. ABRG5-3
Pseudanabaena sp. BC1403
Pseudanabaena sp. Chao 1811
Pseudanabaena sp. FACHB-1050
Pseudanabaena sp. FACHB-1277
Pseudanabaena sp. FACHB-1998
Pseudanabaena sp. FACHB-2040
Pseudanabaena sp. FACHB-723
Pseudanabaena sp. LEGE 07190
Pseudanabaena sp. PCC 6802
Pseudanabaena sp. PCC 7367
Pseudanabaena sp. PCC 7403
Pseudanabaena sp. PCC 9015
Pseudanabaena sp. PCC 9016
Pseudanabaena sp. SCSIO S3
Pseudanabaena sp. SR411
Pseudanabaena sp. UWO310
Pseudanabaena sp. UWO311
Pseudanabaena sp. lw0831
Pseudanabaena sp.
Pseudanabaena yagii
Pseudarthrobacter humi
Pseudarthrobacter oxydans
Pseudarthrobacter polychromogenes
Pseudarthrobacter psychrotolerans
Pseudarthrobacter scleromae
Pseudarthrobacter sp. AB1
Pseudarthrobacter sp. AG30
Pseudarthrobacter sp. BIM B-2242
Pseudarthrobacter sp. HLT1-5
Pseudarthrobacter sp. HLT3-5
Pseudarthrobacter sp. MDT3-9
Pseudarthrobacter sp. MM222
Pseudarthrobacter sp. NBSH8
Pseudarthrobacter sp. NCCP-2145
Pseudarthrobacter sp. NIBRBAC000502772
Pseudarthrobacter sp. SSS035
Pseudarthrobacter sulfonivorans
Pseudenhygromyxa sp. WMMC2535
Pseudidiomarina donghaiensis
Pseudidiomarina halophila
Pseudidiomarina homiensis
Pseudidiomarina sp. CB1
Pseudidiomarina woesei
Pseudidiomarina
Pseudoalteromonas agarivorans
Pseudoalteromonas arctica
Pseudoalteromonas atlantica T6c
Pseudoalteromonas aurantia
Pseudoalteromonas denitrificans DSM 6059
Pseudoalteromonas denitrificans
Pseudoalteromonas espejiana
Pseudoalteromonas flavipulchra
Pseudoalteromonas luteoviolacea B = ATCC 29581
Pseudoalteromonas maricaloris
Pseudoalteromonas phenolica
Pseudoalteromonas piratica
Pseudoalteromonas piscicida
Pseudoalteromonas porphyrae
Pseudoalteromonas rubra
Pseudoalteromonas sp. 2CM41L
Pseudoalteromonas sp. BMB
Pseudoalteromonas sp. BSi20311
Pseudoalteromonas sp. BSi20652
Pseudoalteromonas sp. C2R02
Pseudoalteromonas sp. CO342X
Pseudoalteromonas sp. DL2-H2.2
Pseudoalteromonas sp. GCY
Pseudoalteromonas sp. H103
Pseudoalteromonas sp. H105
Pseudoalteromonas sp. H71
Pseudoalteromonas sp. HM-SA03
Pseudoalteromonas sp. JC3
Pseudoalteromonas sp. MMG024
Pseudoalteromonas sp. NC201
Pseudoalteromonas sp. OOF1S-7
Pseudoalteromonas sp. P1-11
Pseudoalteromonas sp. P1-7a
Pseudoalteromonas sp. P1-9
Pseudoalteromonas sp. R3
Pseudoalteromonas sp. S1609
Pseudoalteromonas sp. S2755
Pseudoalteromonas sp. S3431
Pseudoalteromonas sp. S3776
Pseudoalteromonas sp. S3785
Pseudoalteromonas sp. SWXJZ94C
Pseudoalteromonas sp. T1lg24
Pseudoalteromonas sp. TB64
Pseudoalteromonas sp. XMcav2-N
Pseudoalteromonas sp. Z9A5
Pseudoalteromonas spongiae
Pseudoalteromonas ulvae
Pseudoalteromonas viridis
Pseudoalteromonas
Pseudobacteriovorax antillogorgiicola
Pseudobacteroides cellulosolvens ATCC 35603 = DSM 2933
Pseudobacteroides cellulosolvens
Pseudobutyrivibrio ruminis DSM 9787
Pseudobutyrivibrio ruminis
Pseudobutyrivibrio sp. 49
Pseudobutyrivibrio sp. ACV-2
Pseudobutyrivibrio sp. AR14
Pseudobutyrivibrio sp. JW11
Pseudobutyrivibrio sp. MD2005
Pseudobutyrivibrio sp. NOR37
Pseudobutyrivibrio sp. UC1225
Pseudobutyrivibrio sp. YE44
Pseudobutyrivibrio xylanivorans DSM 14809
Pseudobutyrivibrio xylanivorans
Pseudobutyrivibrio
Pseudochelatococcus contaminans
Pseudochrobactrum sp. AO15b
Pseudochryseolinea flava
Pseudoclavibacter chungangensis
Pseudoclavibacter helvolus
Pseudoclavibacter soli
Pseudoclavibacter sp. 13-3
Pseudoclavibacter sp. AY1F1
Pseudoclavibacter sp. CFCC 11306
Pseudoclavibacter sp. CFCC 13796
Pseudoclavibacter sp. JAI123
Pseudoclavibacter sp. RFBA6
Pseudoclavibacter sp. RFBB5
Pseudoclavibacter sp. RFBG4
Pseudoclavibacter terrae
Pseudoclostridium thermosuccinogenes
Pseudodesulfovibrio aespoeensis Aspo-2
Pseudodesulfovibrio aespoeensis
Pseudodesulfovibrio alkaliphilus
Pseudodesulfovibrio cachew
Pseudodesulfovibrio hydrargyri
Pseudodesulfovibrio indicus
Pseudodesulfovibrio mercurii
Pseudodesulfovibrio piezophilus C1TLV30
Pseudodesulfovibrio piezophilus
Pseudodesulfovibrio portus
Pseudodesulfovibrio profundus
Pseudodesulfovibrio sediminis
Pseudodesulfovibrio senegalensis
Pseudodesulfovibrio sp. MCM B-1480
Pseudodesulfovibrio sp. S3
Pseudodesulfovibrio sp. SYK
Pseudodesulfovibrio sp. zrk46
Pseudodesulfovibrio tunisiensis
Pseudoduganella albidiflava
Pseudoduganella aquatica
Pseudoduganella armeniaca
Pseudoduganella buxea
Pseudoduganella dura
Pseudoduganella flava
Pseudoduganella ginsengisoli
Pseudoduganella lurida
Pseudoduganella lutea
Pseudoduganella namucuonensis
Pseudoduganella plicata
Pseudoduganella rivuli
Pseudoduganella umbonata
Pseudoduganella violaceinigra
Pseudoflavonifractor sp. 60
Pseudoflavonifractor sp. MSJ-37
Pseudogemmobacter bohemicus
Pseudogulbenkiania sp. MAI-1
Pseudogulbenkiania subflava DSM 22618
Pseudogulbenkiania subflava
Pseudohongiella spirulinae
Pseudolabrys sp. FHR47
Pseudolabrys taiwanensis
Pseudolysobacter antarcticus
Pseudomaricurvus alkylphenolicus
Pseudomaricurvus sp. HS19
Pseudomonadaceae
Pseudomonadales bacterium RIFCSPHIGHO2_01_FULL_64_12
Pseudomonadota bacterium
Pseudomonas aeruginosa ATCC 25324
Pseudomonas aeruginosa BL04
Pseudomonas aeruginosa BWHPSA009
Pseudomonas aeruginosa PA21_ST175
Pseudomonas aeruginosa PA38182
Pseudomonas aeruginosa PAO581
Pseudomonas aeruginosa S35004
Pseudomonas aeruginosa WS394
Pseudomonas aeruginosa
Pseudomonas agarici
Pseudomonas alcaligenes
Pseudomonas amygdali pv. mori str. 301020
Pseudomonas amygdali pv. tabaci
Pseudomonas amygdali
Pseudomonas anguilliseptica
Pseudomonas argentinensis
Pseudomonas aromaticivorans
Pseudomonas arsenicoxydans
Pseudomonas asiatica
Pseudomonas avellanae
Pseudomonas baltica
Pseudomonas batumici
Pseudomonas benzenivorans
Pseudomonas bohemica
Pseudomonas brassicacearum
Pseudomonas brassicae
Pseudomonas campi
Pseudomonas canadensis
Pseudomonas cannabina
Pseudomonas capeferrum
Pseudomonas caricapapayae
Pseudomonas carnis
Pseudomonas ceruminis
Pseudomonas chlororaphis
Pseudomonas coleopterorum
Pseudomonas congelans
Pseudomonas coronafaciens pv. coronafaciens
Pseudomonas coronafaciens pv. garcae
Pseudomonas coronafaciens pv. oryzae
Pseudomonas coronafaciens pv. porri
Pseudomonas coronafaciens
Pseudomonas corrugata
Pseudomonas cremoricolorata
Pseudomonas cyclaminis
Pseudomonas daroniae
Pseudomonas defluvii
Pseudomonas donghuensis
Pseudomonas dryadis
Pseudomonas edaphica
Pseudomonas endophytica
Pseudomonas entomophila
Pseudomonas eucalypticola
Pseudomonas fakonensis
Pseudomonas faucium
Pseudomonas flavescens
Pseudomonas flexibilis
Pseudomonas fluorescens ABAC62
Pseudomonas fluorescens HK44
Pseudomonas fluorescens WH6
Pseudomonas fluorescens
Pseudomonas fluvialis
Pseudomonas fragi
Pseudomonas frederiksbergensis
Pseudomonas fulva
Pseudomonas fuscovaginae
Pseudomonas gingeri
Pseudomonas guryensis
Pseudomonas helleri
Pseudomonas hunanensis
Pseudomonas japonica
Pseudomonas jessenii
Pseudomonas juntendi
Pseudomonas kermanshahensis
Pseudomonas kilonensis
Pseudomonas kurunegalensis
Pseudomonas kuykendallii
Pseudomonas lactis
Pseudomonas laoshanensis
Pseudomonas laurentiana
Pseudomonas linyingensis
Pseudomonas lopnurensis
Pseudomonas lutea
Pseudomonas luteola
Pseudomonas mandelii JR-1
Pseudomonas mangiferae
Pseudomonas marginalis
Pseudomonas massiliensis
Pseudomonas matsuisoli
Pseudomonas meliae
Pseudomonas mendocina
Pseudomonas monteilii
Pseudomonas mosselii
Pseudomonas multiresinivorans
Pseudomonas muyukensis
Pseudomonas nanhaiensis
Pseudomonas nicosulfuronedens
Pseudomonas nitritireducens
Pseudomonas nitroreducens
Pseudomonas oleovorans
Pseudomonas oryzae
Pseudomonas oryzagri
Pseudomonas oryzicola
Pseudomonas oryzihabitans
Pseudomonas oryziphila
Pseudomonas otitidis
Pseudomonas ovata
Pseudomonas palleroniana
Pseudomonas parafulva
Pseudomonas petroselini
Pseudomonas pharmacofabricae
Pseudomonas plecoglossicida
Pseudomonas promysalinigenes
Pseudomonas prosekii
Pseudomonas protegens
Pseudomonas pseudonitroreducens
Pseudomonas psychrophila
Pseudomonas psychrotolerans L19
Pseudomonas psychrotolerans
Pseudomonas punonensis
Pseudomonas putida H8234
Pseudomonas putida NBRC 14164
Pseudomonas putida ND6
Pseudomonas putida S16
Pseudomonas putida group
Pseudomonas putida
Pseudomonas quasicaspiana
Pseudomonas resinovorans NBRC 106553
Pseudomonas resinovorans
Pseudomonas rhizoryzae
Pseudomonas rhizosphaerae
Pseudomonas rhodesiae
Pseudomonas sagittaria
Pseudomonas salomonii
Pseudomonas saudimassiliensis
Pseudomonas saudiphocaensis
Pseudomonas savastanoi pv. fraxini
Pseudomonas savastanoi pv. glycinea
Pseudomonas savastanoi pv. nerii
Pseudomonas savastanoi pv. phaseolicola 1448A
Pseudomonas savastanoi
Pseudomonas segetis
Pseudomonas seleniipraecipitans
Pseudomonas sichuanensis
Pseudomonas sihuiensis
Pseudomonas sivasensis
Pseudomonas sp. 008
Pseudomonas sp. 10-1B
Pseudomonas sp. 16_A
Pseudomonas sp. 17391
Pseudomonas sp. 1D4
Pseudomonas sp. 21LCFQ010
Pseudomonas sp. 21LCFQ02
Pseudomonas sp. 21
Pseudomonas sp. 24 E 13
Pseudomonas sp. 2822-15
Pseudomonas sp. 2VD
Pseudomonas sp. 32.2.56
Pseudomonas sp. 32_A
Pseudomonas sp. 39004
Pseudomonas sp. 51_B
Pseudomonas sp. 58 R 3
Pseudomonas sp. 79_C
Pseudomonas sp. 8BK
Pseudomonas sp. A-1
Pseudomonas sp. A2
Pseudomonas sp. AAC
Pseudomonas sp. ABC1
Pseudomonas sp. AF-4B
Pseudomonas sp. AFG_SD02_1510_Pfu_092
Pseudomonas sp. AG1028
Pseudomonas sp. AS2.8
Pseudomonas sp. Ant30-3
Pseudomonas sp. B10(2017)
Pseudomonas sp. B14(2017)
Pseudomonas sp. B21-031
Pseudomonas sp. B21-047
Pseudomonas sp. B4(2012c)
Pseudomonas sp. B8(2017)
Pseudomonas sp. BAY1663
Pseudomonas sp. BMS12
Pseudomonas sp. BT-42-2
Pseudomonas sp. Bi70
Pseudomonas sp. C5pp
Pseudomonas sp. CC120222-01a
Pseudomonas sp. CCBAU 101047
Pseudomonas sp. CCBAU 10909
Pseudomonas sp. CCOS 191
Pseudomonas sp. CFA
Pseudomonas sp. CFBP 13602
Pseudomonas sp. CFBP 13710
Pseudomonas sp. CFBP 13719
Pseudomonas sp. CFBP 13727
Pseudomonas sp. CFBP 8758
Pseudomonas sp. CFBP 8771
Pseudomonas sp. CFII64
Pseudomonas sp. CIP-10
Pseudomonas sp. CM25
Pseudomonas sp. CM27
Pseudomonas sp. CNPSo 3701
Pseudomonas sp. Ch10048
Pseudomonas sp. D5-2
Pseudomonas sp. D5
Pseudomonas sp. DCB_AW
Pseudomonas sp. DCB_BI
Pseudomonas sp. DC
Pseudomonas sp. DE0010
Pseudomonas sp. DP-17
Pseudomonas sp. DP16D-T1
Pseudomonas sp. EGD-AK9
Pseudomonas sp. EKM23D
Pseudomonas sp. ES3-33
Pseudomonas sp. EpS/L25
Pseudomonas sp. F1002
Pseudomonas sp. FFUP_PS_473
Pseudomonas sp. FGI182
Pseudomonas sp. FW 305-130
Pseudomonas sp. FW306-02-H05-AB
Pseudomonas sp. FW306-2-11BA
Pseudomonas sp. FeS53a
Pseudomonas sp. Fl4BN2
Pseudomonas sp. GCEP-101
Pseudomonas sp. GM102
Pseudomonas sp. GM17
Pseudomonas sp. GM18
Pseudomonas sp. GM33
Pseudomonas sp. GM49
Pseudomonas sp. GM50
Pseudomonas sp. GM55
Pseudomonas sp. GM79
Pseudomonas sp. GM80
Pseudomonas sp. GM84
Pseudomonas sp. GM_Psu_2
Pseudomonas sp. GR 6-02
Pseudomonas sp. GW101-1A09
Pseudomonas sp. Gutcm_11s
Pseudomonas sp. HLS-6
Pseudomonas sp. HMWF010
Pseudomonas sp. HMWF011
Pseudomonas sp. HMWF031
Pseudomonas sp. HMWF032
Pseudomonas sp. HN2-3
Pseudomonas sp. HPB0071
Pseudomonas sp. HS-18
Pseudomonas sp. ICMP 561
Pseudomonas sp. IPPW-2
Pseudomonas sp. IPPW-3
Pseudomonas sp. Irchel 3A5
Pseudomonas sp. Irchel 3F3
Pseudomonas sp. Irchel s3f19
Pseudomonas sp. J452
Pseudomonas sp. JBR1
Pseudomonas sp. JR33AA
Pseudomonas sp. K1-2004
Pseudomonas sp. K35
Pseudomonas sp. KBS0710
Pseudomonas sp. KNUC1026
Pseudomonas sp. Kh13
Pseudomonas sp. LA5
Pseudomonas sp. LAIL14HWK12: I4
Pseudomonas sp. LAIL14HWK12: I7
Pseudomonas sp. LB-090624
Pseudomonas sp. LRP2-20
Pseudomonas sp. Leaf127
Pseudomonas sp. Leaf129
Pseudomonas sp. Leaf48
Pseudomonas sp. M30-35
Pseudomonas sp. M47T1
Pseudomonas sp. M5
Pseudomonas sp. ML96
Pseudomonas sp. MM221
Pseudomonas sp. MN1F
Pseudomonas sp. MNR3A
Pseudomonas sp. MOIL14HWK12: I2
Pseudomonas sp. MPBD7-1
Pseudomonas sp. MRSN 12121
Pseudomonas sp. MS15a(2019)
Pseudomonas sp. MWU13-3659
Pseudomonas sp. MWU16-30317
Pseudomonas sp. Marseille-Q3773
Pseudomonas sp. Marseille-Q5115
Pseudomonas sp. Marseille-Q5299
Pseudomonas sp. Mf238
Pseudomonas sp. N040
Pseudomonas sp. N2-11
Pseudomonas sp. NBRC 111118
Pseudomonas sp. NBRC 111119
Pseudomonas sp. NBRC 111120
Pseudomonas sp. NBRC 111124
Pseudomonas sp. NBRC 111125
Pseudomonas sp. NBRC 111126
Pseudomonas sp. NBRC 111127
Pseudomonas sp. NBRC 111131
Pseudomonas sp. NBRC 111132
Pseudomonas sp. NBRC 111133
Pseudomonas sp. NBRC 111140
Pseudomonas sp. NMI4491_12
Pseudomonas sp. NMI542_15
Pseudomonas sp. NY5710
Pseudomonas sp. OE 28.3
Pseudomonas sp. OF001
Pseudomonas sp. P6-16
Pseudomonas sp. P7-14
Pseudomonas sp. PA-1-2A
Pseudomonas sp. PA-3-6H
Pseudomonas sp. PA1(2017)
Pseudomonas sp. PA15(2017)
Pseudomonas sp. PA27(2017)
Pseudomonas sp. PAGU 2196
Pseudomonas sp. PDM11
Pseudomonas sp. PDM12
Pseudomonas sp. PDM14
Pseudomonas sp. PDM17
Pseudomonas sp. PDM18
Pseudomonas sp. PDM19
Pseudomonas sp. PDM20
Pseudomonas sp. PDM33
Pseudomonas sp. PDNC002
Pseudomonas sp. PGPR81
Pseudomonas sp. PIA16
Pseudomonas sp. PLB05
Pseudomonas sp. PS1
Pseudomonas sp. Pc102
Pseudomonas sp. QTF5
Pseudomonas sp. R-46240
Pseudomonas sp. R1-73
Pseudomonas sp. R62
Pseudomonas sp. RIT 411
Pseudomonas sp. RIT-PI-a
Pseudomonas sp. RW3S2
Pseudomonas sp. RtIB026
Pseudomonas sp. S31
Pseudomonas sp. S32
Pseudomonas sp. S35
Pseudomonas sp. S36
Pseudomonas sp. S37
Pseudomonas sp. S44
Pseudomonas sp. S5F11
Pseudomonas sp. S60
Pseudomonas sp. S9
Pseudomonas sp. SB2
Pseudomonas sp. SK
Pseudomonas sp. SL4(2022)
Pseudomonas sp. SWI36
Pseudomonas sp. SWI7
Pseudomonas sp. SWRI50
Pseudomonas sp. SWRI77
Pseudomonas sp. Sample_10
Pseudomonas sp. Sample_24
Pseudomonas sp. Sample_9
Pseudomonas sp. StFLB209
Pseudomonas sp. T1-3-2
Pseudomonas sp. T16(2010)
Pseudomonas sp. TJI-51
Pseudomonas sp. TMW 2.1634
Pseudomonas sp. TNT11
Pseudomonas sp. TTU2014-080ASC
Pseudomonas sp. UFMG81
Pseudomonas sp. UMAB-08
Pseudomonas sp. URMO17WK12: I11
Pseudomonas sp. URMO17WK12: I1
Pseudomonas sp. URMO17WK12: I2
Pseudomonas sp. URMO17WK12: I3
Pseudomonas sp. URMO17WK12: I4
Pseudomonas sp. UW4
Pseudomonas sp. WS 5146
Pseudomonas sp. XJUHX-12
Pseudomonas sp. XWY-1
Pseudomonas sp. Xaverov 259
Pseudomonas sp. YIM B01952
Pseudomonas sp. YY-1
Pseudomonas sp. dw_358
Pseudomonas sp. gx-127
Pseudomonas sp. p106
Pseudomonas sp. p99-361
Pseudomonas sp. sia0905
Pseudomonas sp. v388
Pseudomonas sp.
Pseudomonas spelaei
Pseudomonas straminea
Pseudomonas syringae BRIP34876
Pseudomonas syringae BRIP34881
Pseudomonas syringae BRIP39023
Pseudomonas syringae group genomosp. 3
Pseudomonas syringae group
Pseudomonas syringae pv. aceris str. M302273
Pseudomonas syringae pv. aceris
Pseudomonas syringae pv. actinidiae ICMP 18804
Pseudomonas syringae pv. actinidiae ICMP 19096
Pseudomonas syringae pv. actinidiae str. M302091
Pseudomonas syringae pv. actinidiae str. Shaanxi_M228
Pseudomonas syringae pv. actinidiae
Pseudomonas syringae pv. aptata
Pseudomonas syringae pv. avellanae str. ISPaVe013
Pseudomonas syringae pv. avii
Pseudomonas syringae pv. coryli
Pseudomonas syringae pv. japonica str. M301072
Pseudomonas syringae pv. maculicola
Pseudomonas syringae pv. papulans
Pseudomonas syringae pv. philadelphi
Pseudomonas syringae pv. pisi
Pseudomonas syringae pv. syringae B301D
Pseudomonas syringae pv. syringae str. B301D-R
Pseudomonas syringae pv. syringae
Pseudomonas syringae pv. tagetis
Pseudomonas syringae pv. tomato str. DC3000
Pseudomonas syringae pv. tomato
Pseudomonas syringae
Pseudomonas taeanensis MS-3
Pseudomonas taetrolens
Pseudomonas taiwanensis
Pseudomonas tohonis
Pseudomonas tolaasii
Pseudomonas trivialis
Pseudomonas tumugii
Pseudomonas turukhanskensis
Pseudomonas typographi
Pseudomonas ullengensis
Pseudomonas urethralis
Pseudomonas urmiensis
Pseudomonas versuta
Pseudomonas viridiflava
Pseudomonas vlassakiae
Pseudomonas weihenstephanensis
Pseudomonas xanthosomae
Pseudomonas xionganensis
Pseudomonas yamanorum
Pseudomonas yangonensis
Pseudomonas
Pseudomyrmex gracilis
Pseudonocardia abyssalis
Pseudonocardia acaciae
Pseudonocardia acidicola
Pseudonocardia alaniniphila
Pseudonocardia alni
Pseudonocardia ammonioxydans
Pseudonocardia antarctica
Pseudonocardia bannensis
Pseudonocardia broussonetiae
Pseudonocardia cypriaca
Pseudonocardia dioxanivorans
Pseudonocardia endophytica
Pseudonocardia hierapolitana
Pseudonocardia hydrocarbonoxydans
Pseudonocardia kujensis
Pseudonocardia nigra
Pseudonocardia oceani
Pseudonocardia oroxyli
Pseudonocardia parietis
Pseudonocardia petroleophila
Pseudonocardia pini
Pseudonocardia sediminis
Pseudonocardia sp. AL041005-10
Pseudonocardia sp. Ae505_Ps2
Pseudonocardia sp. Ae707_Ps1
Pseudonocardia sp. Ae717_Ps2
Pseudonocardia sp. C8
Pseudonocardia sp. CNS-139
Pseudonocardia sp. DR1-2
Pseudonocardia sp. DSM 110487
Pseudonocardia sp. EV170527-09
Pseudonocardia sp. H11422
Pseudonocardia sp. H11425
Pseudonocardia sp. HH130629-09
Pseudonocardia sp. HH130630-07
Pseudonocardia sp. ICBG1034
Pseudonocardia sp. ICBG1142
Pseudonocardia sp. ICBG1293
Pseudonocardia sp. ICBG601
Pseudonocardia sp. KRD291
Pseudonocardia sp. MH-G8
Pseudonocardia sp. McavD-2-B
Pseudonocardia sp. SID8383
Pseudonocardia sp. WMMC193
Pseudonocardia spinosispora
Pseudonocardia sulfidoxydans
Pseudonocardia terrae
Pseudonocardia thermophila
Pseudonocardia xinjiangensis
Pseudonocardia
Pseudonocardiales bacterium
Pseudooceanicola lipolyticus
Pseudooceanicola nanhaiensis
Pseudooceanicola
Pseudooctadecabacter jejudonensis
Pseudophaeobacter flagellatus
Pseudophaeobacter leonis
Pseudopontixanthobacter vadosimaris
Pseudoramibacter alactolyticus ATCC 23263
Pseudoramibacter alactolyticus
Pseudoramibacter porci
Pseudorhizobium banfieldiae
Pseudorhizobium flavum
Pseudorhizobium pelagicum
Pseudorhizobium tarimense
Pseudorhodobacter aquimaris
Pseudorhodobacter sp. MZDSW-24AT
Pseudorhodobacter sp. PARRP1
Pseudorhodoferax aquiterrae
Pseudorhodoferax soli
Pseudorhodoferax sp. Leaf265
Pseudorhodoferax sp. Leaf267
Pseudorhodoferax sp. Leaf274
Pseudorhodoplanes sinuspersici
Pseudoruegeria aquimaris
Pseudoruegeria marinistellae
Pseudoruminococcus massiliensis
Pseudospirillum japonicum
Pseudosulfitobacter sp. AP-MA-4
Pseudoteredinibacter isoporae
Pseudothauera hydrothermalis
Pseudothauera lacus
Pseudothauera nasutitermitis
Pseudothauera rhizosphaerae
Pseudothermotoga hypogea DSM 11164 = NBRC 106472
Pseudothermotoga hypogea
Pseudothermotoga lettingae TMO
Pseudothermotoga thermarum DSM 5069
Pseudothermotoga thermarum
Pseudothioclava arenosa
Pseudovibrio exalbescens
Pseudoxanthomonas composti
Pseudoxanthomonas dokdonensis
Pseudoxanthomonas indica
Pseudoxanthomonas kaohsiungensis
Pseudoxanthomonas sp. GM95
Pseudoxanthomonas sp. GW2
Pseudoxanthomonas sp. J31
Pseudoxanthomonas sp. J35
Pseudoxanthomonas sp. JBR18
Pseudoxanthomonas sp. Root630
Pseudoxanthomonas sp. Root65
Pseudoxanthomonas sp. SGNA-20
Pseudoxanthomonas sp. SGT-18
Pseudoxanthomonas sp. X-1
Pseudoxanthomonas spadix
Pseudoxanthomonas suwonensis 11-1
Pseudoxanthomonas suwonensis
Pseudoxanthomonas taiwanensis
Pseudoxanthomonas winnipegensis
Pseudozyma hubeiensis SY62
Psychrilyobacter atlanticus
Psychrobacillus psychrodurans
Psychrobacillus sp. FJAT-21963
Psychrobacter aquaticus CMS 56
Psychrobacter aquaticus
Psychrobacter arcticus 273-4
Psychrobacter arcticus
Psychrobacter piscatorii
Psychrobacter sp. DAB AL43B
Psychrobacter sp. JCM 18900
Psychrobacter sp. JCM 18901
Psychrobacter sp. P11F6
Psychrobacter sp. P11G5
Psychrobacter urativorans
Psychrode sulfovibrio sp. FT415
Psychroflexus aestuariivivens
Psychroflexus curvus
Psychromicrobium lacuslunae
Psychromicrobium silvestre
Psychromonas antarctica
Psychromonas arctica
Psychromonas hadalis
Psychromonas marina
Psychromonas sp. CNPT3
Psychromonas
Psychrosphaera ytuae
Puccinia coronata f. sp. avenae
Puccinia striiformis
Puerhibacterium puerhi
Punica granatum
Puniceibacterium antarcticum
Puniceicoccaceae bacterium 5H
Puniceicoccus vermicola
Pusillimonas faecipullorum
Pyramidobacter piscolens W5455
Pyramidobacter piscolens
Pyramidobacter sp. C12-8
Pyramidobacter sp. CG50-2
Pyrinomonas methylaliphatogenes
Pyrobaculum aerophilum str. IM2
Pyrobaculum aerophilum
Pyrococcus horikoshii OT3
Pyxidicoccus caerfyrddinensis
Pyxidicoccus fallax
Pyxidicoccus sp. MSG2
Pyxidicoccus sp. SCPEA002
Pyxidicoccus xibeiensis
Qipengyuania aquimaris
Qipengyuania citrea
Qipengyuania gaetbuli
Qipengyuania intermedia
Qipengyuania nanhaisediminis
Qipengyuania pelagi
Qipengyuania seohaensis
Qipengyuania vulgaris
Quadrisphaera granulorum
Quadrisphaera sp. RL12-1S
Quatrionicoccus australiensis
Quercus suber
Rahnella aceris
Rahnella aquatilis CIP 78.65 = ATCC 33071
Rahnella aquatilis HX2
Rahnella aquatilis
Rahnella perminowiae
Rahnella sp. AA
Rahnella sp. BIGb0236
Rahnella sp. RcJ3
Rahnella sp. WP5
Rahnella victoriana
Rahnella
Ralstonia pseudosolanacearum
Ralstonia solanacearum UW551
Ralstonia solanacearum
Ralstonia sp. 25mfcol4.1
Ralstonia sp. ISSDS-784
Ralstonia sp. S1SM82
Ramlibacter agri
Ramlibacter ginsenosidimutans
Ramlibacter humi
Ramlibacter sp. B156
Ramlibacter sp. GTP1
Ramlibacter sp. HM2
Ramlibacter sp. USB13
Ramlibacter tatouinenis
Ramlibacter
Raoultella electrica
Raoultella ornithinolytica
Raoultella planticola
Raoultella sp. 10-1
Raoultella sp. Lac2
Raoultella sp. NCTC 9187
Raoultella sp. T31
Raoultella terrigena
Raoultella
Raoultibacter massiliensis
Raoultibacter sp. Marseille-P8396
Raoultibacter timonensis
Raphidiopsis brookii D9
Raphidocelis subcapitata
Rathayibacter sp. AY1D7
Reichenbachiella agariperforans
Reichenbachiella faecimaris
Reichenbachiella sp. ABR2-5
Reichenbachiella sp. BKB1-1
Reinekea blandensis
Reinekea forsetii
Reinekea marinisedimentorum
Reinekea sp. G2M2-21
Reinekea sp. MED297
Reinekea thalattae
Reyranella aquatilis
Reyranella sp. MMS21-HV4-11
Rhabdochromatium marinum
Rheinheimera hassiensis
Rheinheimera nanhaiensis E407-8
Rheinheimera riviphila
Rheinheimera sp. 4Y26
Rheinheimera sp. A13L
Rheinheimera sp. EpRS3
Rheinheimera sp. F8
Rheinheimera sp. KL1
Rheinheimera sp. SA_1
Rhizobacter sp. AJA081-3
Rhizobacter sp. Root1221
Rhizobacter sp. Root404
Rhizobiaceae
Rhizobiales bacterium 12-66-7
Rhizobiales bacterium 24-66-13
Rhizobiales bacterium 35-66-30
Rhizobiales bacterium 39-66-18
Rhizobiales bacterium GAS113
Rhizobiales bacterium GAS188
Rhizobiales bacterium GAS191
Rhizobiales bacterium TMED143
Rhizobiales bacterium TMED29
Rhizobiales bacterium ltg2
Rhizobium acidisoli
Rhizobium aegyptiacum
Rhizobium aethiopicum
Rhizobium alamii
Rhizobium album
Rhizobium altiplani
Rhizobium anhuiense bv. trifolii
Rhizobium anhuiense
Rhizobium azibense
Rhizobium bangladeshense
Rhizobium binae
Rhizobium calliandrae
Rhizobium cellulosilyticum
Rhizobium changzhiense
Rhizobium chutanense
Rhizobium cremeum
Rhizobium daejeonense
Rhizobium deserti
Rhizobium esperanzae
Rhizobium etli 8C-3
Rhizobium etli CFN 42
Rhizobium etli CIAT 652
Rhizobium etli CNPAF512
Rhizobium etli bv. mimosae str. Mim1
Rhizobium etli bv. mimosae
Rhizobium etli bv. phaseoli str. IE4803
Rhizobium etli bv. phaseoli
Rhizobium etli
Rhizobium fabae
Rhizobium favelukesii
Rhizobium freirei PRF 81
Rhizobium gallicum bv. gallicum R602sp
Rhizobium gallicum bv. gallicum
Rhizobium gallicum bv. phaseoli
Rhizobium gallicum
Rhizobium giardinii bv. giardinii
Rhizobium giardinii bv. phaseoli
Rhizobium giardinii
Rhizobium glycinendophyticum
Rhizobium grahamii CCGE 502
Rhizobium grahamii
Rhizobium hainanense
Rhizobium halophilum
Rhizobium halophytocola
Rhizobium herbae
Rhizobium hidalgonense
Rhizobium indicum
Rhizobium indigoferae
Rhizobium jaguaris
Rhizobium laguerreae
Rhizobium leguminosarum bv. phaseoli CCGM1
Rhizobium leguminosarum bv. phaseoli
Rhizobium leguminosarum bv. trifolii CB782
Rhizobium leguminosarum bv. trifolii TA1
Rhizobium leguminosarum bv. trifolii WSM1325
Rhizobium leguminosarum bv. trifolii WSM1689
Rhizobium leguminosarum bv. trifolii WSM2012
Rhizobium leguminosarum bv. trifolii WSM2297
Rhizobium leguminosarum bv. trifolii WSM2304
Rhizobium leguminosarum bv. trifolii WSM597
Rhizobium leguminosarum bv. trifolii
Rhizobium leguminosarum bv. viciae 248
Rhizobium leguminosarum bv. viciae 3841
Rhizobium leguminosarum bv. viciae USDA 2370
Rhizobium leguminosarum bv. viciae WSM1455
Rhizobium leguminosarum bv. viciae
Rhizobium leguminosarum
Rhizobium lentis
Rhizobium leucaenae
Rhizobium lusitanum
Rhizobium mayense
Rhizobium mesoamericanum CCGE 501
Rhizobium mesoamericanum STM3625
Rhizobium mesoamericanum
Rhizobium mesosinicum
Rhizobium miluonense
Rhizobium mongolense USDA 1844
Rhizobium mongolense subsp. loessense
Rhizobium mongolense
Rhizobium multihospitium
Rhizobium nepotum 39/7
Rhizobium phaseoli Brasil 5
Rhizobium phaseoli Ch24-10
Rhizobium phaseoli
Rhizobium pisi
Rhizobium populi
Rhizobium populisoli
Rhizobium pusense
Rhizobium gilianshanense
Rhizobium rhizophilum
Rhizobium rosettiformans W3
Rhizobium ruizarguesonis
Rhizobium skierniewicense
Rhizobium smilacinae
Rhizobium sophorae
Rhizobium sophoriradicis
Rhizobium sp. 007
Rhizobium sp. 05973
Rhizobium sp. 1532
Rhizobium sp. 1556
Rhizobium sp. 1587
Rhizobium sp. 1617
Rhizobium sp. 1627
Rhizobium sp. 1648
Rhizobium sp. 1652
Rhizobium sp. 1706
Rhizobium sp. 23C55
Rhizobium sp. 2MFCol3.1
Rhizobium sp. 43-2-1
Rhizobium sp. 43-3-1
Rhizobium sp. 52-3-1
Rhizobium sp. 525W
Rhizobium sp. 53-1-1
Rhizobium sp. 53-2-1
Rhizobium sp. AC100a
Rhizobium sp. AC100b
Rhizobium sp. AC4
Rhizobium sp. AC80a
Rhizobium sp. AC80b
Rhizobium sp. AC80c
Rhizobium sp. AC80d
Rhizobium sp. AC80e
Rhizobium sp. AC82b
Rhizobium sp. AC87k1
Rhizobium sp. AC87k3
Rhizobium sp. AC87k4
Rhizobium sp. AC89a
Rhizobium sp. AC89b
Rhizobium sp. AC89c
Rhizobium sp. AC89d
Rhizobium sp. AC89e
Rhizobium sp. AC90e1
Rhizobium sp. AC91a
Rhizobium sp. AC91c
Rhizobium sp. AC91e
Rhizobium sp. AC93b
Rhizobium sp. AC93d
Rhizobium sp. ACO-34A
Rhizobium sp. AG855
Rhizobium sp. AM1
Rhizobium sp. AM5
Rhizobium sp. ANU289
Rhizobium sp. ATQ2
Rhizobium sp. AngicoI_417
Rhizobium sp. BAM-1
Rhizobium sp. BAM-2
Rhizobium sp. BAM-3
Rhizobium sp. BAM-4
Rhizobium sp. BAM-5
Rhizobium sp. BAM-6
Rhizobium sp. BAM-7
Rhizobium sp. BAM-8
Rhizobium sp. BG4
Rhizobium sp. BK661
Rhizobium sp. BR 10427
Rhizobium sp. BR 10432
Rhizobium sp. BR6001
Rhizobium sp. C104
Rhizobium sp. CCBAU 03360
Rhizobium sp. CCBAU 03409
Rhizobium sp. CCBAU 10881
Rhizobium sp. CCBAU 10885
Rhizobium sp. CCBAU 10891
Rhizobium sp. CCBAU 23250
Rhizobium sp. CCBAU 23251
Rhizobium sp. CCBAU 23258
Rhizobium sp. CCBAU 23261
Rhizobium sp. CCBAU 33220A
Rhizobium sp. CCBAU 33498
Rhizobium sp. CCBAU 33499
Rhizobium sp. CCBAU 33502
Rhizobium sp. CCBAU 33510
Rhizobium sp. CCBAU 33511
Rhizobium sp. CCBAU 33518
Rhizobium sp. CCBAU 33529
Rhizobium sp. CCBAU 33532
Rhizobium sp. CCBAU 33563
Rhizobium sp. CCBAU 33585
Rhizobium sp. CCBAU 33587
Rhizobium sp. CCBAU 33592
Rhizobium sp. CCBAU 33601
Rhizobium sp. CCBAU 45499
Rhizobium sp. CCBAU 65118
Rhizobium sp. CCBAU 65255
Rhizobium sp. CCBAU 65813
Rhizobium sp. CCBAU 83266
Rhizobium sp. CCBAU 83268
Rhizobium sp. CCBAU 83309
Rhizobium sp. CCBAU 83452
Rhizobium sp. CCBAU 83457
Rhizobium sp. CCBAU 83475
Rhizobium sp. CCBAU 83476
Rhizobium sp. CCBAU 83477
Rhizobium sp. CCBAU 83480
Rhizobium sp. CCBAU 83482
Rhizobium sp. CCBAU 83485
Rhizobium sp. CCBAU 83489
Rhizobium sp. CCBAU 83490
Rhizobium sp. CCBAU 83491
Rhizobium sp. CCBAU 83514
Rhizobium sp. CCBAU 83515
Rhizobium sp. CCBAU 83517
Rhizobium sp. CCBAU 83526
Rhizobium sp. CCBAU 83530
Rhizobium sp. CCGE 510
Rhizobium sp. CCGE531
Rhizobium sp. CCGE532
Rhizobium sp. CCNWGS0022
Rhizobium sp. CCNWSX0011-1
Rhizobium sp. CCNWSX0878
Rhizobium sp. CCNWSX0879
Rhizobium sp. CCNWSX0884
Rhizobium sp. CCNWSX0887
Rhizobium sp. CCNWSX1016
Rhizobium sp. CECT 9324
Rhizobium sp. CF122
Rhizobium sp. CFBP 8752
Rhizobium sp. CFBP 8762
Rhizobium sp. CFN ESH34
Rhizobium sp. CIAT894
Rhizobium sp. CIR16B
Rhizobium sp. CPSR4B
Rhizobium sp. CRIBSB
Rhizobium sp. CSR8B
Rhizobium sp. CTG-403
Rhizobium sp. CTG-407
Rhizobium sp. CTG-412
Rhizobium sp. CTG-416
Rhizobium sp. CTG-419
Rhizobium sp. CTG-423
Rhizobium sp. CTG-427
Rhizobium sp. CTG-430
Rhizobium sp. Cs217
Rhizobium sp. DASA 68006
Rhizobium sp. DKSPLA3
Rhizobium sp. ERR7
Rhizobium sp. FB10061
Rhizobium sp. FB1202
Rhizobium sp. FB1206
Rhizobium sp. FB2405
Rhizobium sp. FB2504
Rhizobium sp. FB9071
Rhizobium sp. FG01
Rhizobium sp. FH20
Rhizobium sp. FK8
Rhizobium sp. FKL33
Rhizobium sp. G21
Rhizobium sp. GR11D
Rhizobium sp. GR12C
Rhizobium sp. GR7A
Rhizobium sp. H4
Rhizobium sp. HBR22
Rhizobium sp. HBR42
Rhizobium sp. HBR79
Rhizobium sp. HG8
Rhizobium sp. HK12
Rhizobium sp. HK7
Rhizobium sp. IB6
Rhizobium sp. ICMP 19433
Rhizobium sp. ICMP 19817
Rhizobium sp. ICMP 19821
Rhizobium sp. ICMP 19857
Rhizobium sp. ICMP 19859
Rhizobium sp. ICMP 19860
Rhizobium sp. ICMP 19862
Rhizobium sp. ICMP 19865
Rhizobium sp. IE4771
Rhizobium sp. J15
Rhizobium sp. JNVU MP10
Rhizobium sp. JPY1075
Rhizobium sp. JPY1166
Rhizobium sp. JPY1198
Rhizobium sp. JPY1206
Rhizobium sp. JPY1252
Rhizobium sp. JPY1269
Rhizobium sp. JPY1318
Rhizobium sp. JPY1359
Rhizobium sp. JPY727
Rhizobium sp. JPY735
Rhizobium sp. JPY783
Rhizobium sp. JPY794
Rhizobium sp. JPY811
Rhizobium sp. JPY820
Rhizobium sp. JPY829
Rhizobium sp. JPY877
Rhizobium sp. JPY888
Rhizobium sp. JPY892
Rhizobium sp. JPY934
Rhizobium sp. JPY940
Rhizobium sp. JPY946
Rhizobium sp. K102
Rhizobium sp. KAs_5_22
Rhizobium sp. KNUC172
Rhizobium sp. KNa4
Rhizobium sp. KNb1
Rhizobium sp. KNb6
Rhizobium sp. KNb9
Rhizobium sp. KNc7
Rhizobium sp. KNd4
Rhizobium sp. Kim5
Rhizobium sp. L18
Rhizobium sp. L43
Rhizobium sp. L9
Rhizobium sp. LBP2-2
Rhizobium sp. Leaf155
Rhizobium sp. Leaf262
Rhizobium sp. Leaf306
Rhizobium sp. Leaf311
Rhizobium sp. Leaf321
Rhizobium sp. Leaf341
Rhizobium sp. Leaf371
Rhizobium sp. Leaf383
Rhizobium sp. Leaf386
Rhizobium sp. Leaf391
Rhizobium sp. Leaf453
Rhizobium sp. Lpe5
Rhizobium sp. M10
Rhizobium sp. M1
Rhizobium sp. MHM7A
Rhizobium sp. Mug-8
Rhizobium sp. N113
Rhizobium sp. N1314
Rhizobium sp. N1341
Rhizobium sp. N324
Rhizobium sp. N4311
Rhizobium sp. N541
Rhizobium sp. N6212
Rhizobium sp. N621
Rhizobium sp. N731
Rhizobium sp. N741
Rhizobium sp. N871
Rhizobium sp. N941
Rhizobium sp. NCHA22
Rhizobium sp. NCSU 1953
Rhizobium sp. NCSU 2076
Rhizobium sp. NCSU 2200
Rhizobium sp. NCSU 2209
Rhizobium sp. NCSU 2363
Rhizobium sp. NCSU 2642
Rhizobium sp. NCSU 2658
Rhizobium sp. NFR07
Rhizobium sp. NG07A
Rhizobium sp. NG07B
Rhizobium sp. NGR181
Rhizobium sp. NLR16a
Rhizobium sp. NXC14
Rhizobium sp. NXC24
Rhizobium sp. NZLR1
Rhizobium sp. OD123
Rhizobium sp. OD53
Rhizobium sp. P38BS-XIX
Rhizobium sp. PEPV16
Rhizobium sp. PP-F2F-G48
Rhizobium sp. PRIMUS64
Rhizobium sp. Pop5
Rhizobium sp. R-45648
Rhizobium sp. R2-708
Rhizobium sp. R339
Rhizobium sp. R45621
Rhizobium sp. R46039
Rhizobium sp. R46140
Rhizobium sp. R46265
Rhizobium sp. R46283
Rhizobium sp. R634
Rhizobium sp. RAS22
Rhizobium sp. RMCC TR1811
Rhizobium sp. RMCC TR1821
Rhizobium sp. RMCC TR1831
Rhizobium sp. RMCC TR2021
Rhizobium sp. RMa-01
Rhizobium sp. RPJ16
Rhizobium sp. RPJ3
Rhizobium sp. RPJ5
Rhizobium sp. RPJ6
Rhizobium sp. RPP14
Rhizobium sp. RPP20
Rhizobium sp. RSm-3
Rhizobium sp. RU33A
Rhizobium sp. RU36D
Rhizobium sp. Root1204
Rhizobium sp. Root708
Rhizobium sp. S1SS148
Rhizobium sp. S9
Rhizobium sp. SA3
Rhizobium sp. SCAUf12
Rhizobium sp. SCAUf15
Rhizobium sp. SCAUf20
Rhizobium sp. SCAUf2
Rhizobium sp. SCAUf32
Rhizobium sp. SCAUf33
Rhizobium sp. SCAUf34
Rhizobium sp. SCAUf38
Rhizobium sp. SCAUf46
Rhizobium sp. SCAUf54
Rhizobium sp. SEMIA 4085
Rhizobium sp. SEMIA 4088
Rhizobium sp. SG14
Rhizobium sp. SL86
Rhizobium sp. SMF 1356_6
Rhizobium sp. SMF 466_6
Rhizobium sp. STM 3625
Rhizobium sp. STM 3696
Rhizobium sp. STM 3716
Rhizobium sp. STM 3719
Rhizobium sp. STM 3730
Rhizobium sp. STM 3735
Rhizobium sp. STM 3945
Rhizobium sp. STM 7300
Rhizobium sp. STM 7321
Rhizobium sp. STM 7330
Rhizobium sp. T136
Rhizobium sp. TAL182
Rhizobium sp. TH135
Rhizobium sp. TJ167
Rhizobium sp. TJ171
Rhizobium sp. TJ172
Rhizobium sp. TJ173
Rhizobium sp. TK16
Rhizobium sp. TN04
Rhizobium sp. TRM95796
Rhizobium sp. TUTAHSA60
Rhizobium sp. TUTPVSA101
Rhizobium sp. TUTPVSA104
Rhizobium sp. TUTPVSA107
Rhizobium sp. TUTPVSA110
Rhizobium sp. TUTPVSA114
Rhizobium sp. TUTPVSA117
Rhizobium sp. TUTPVSA121
Rhizobium sp. TUTPVSA124
Rhizobium sp. TUTPVSA125
Rhizobium sp. TUTPVSA128
Rhizobium sp. TUTPVSA16
Rhizobium sp. TUTPVSA18
Rhizobium sp. TUTPVSA21
Rhizobium sp. TUTPVSA24
Rhizobium sp. TUTPVSA30
Rhizobium sp. TUTPVSA41
Rhizobium sp. TUTPVSA42
Rhizobium sp. TUTPVSA5
Rhizobium sp. TUTPVSA6
Rhizobium sp. TUTPVSA81
Rhizobium sp. TYa3
Rhizobium sp. TYb10
Rhizobium sp. TYb1
Rhizobium sp. TYb6
Rhizobium sp. USDA 1920
Rhizobium sp. UYPR7.63
Rhizobium sp. WL3
Rhizobium sp. WW_1
Rhizobium sp. WYCCWR10002
Rhizobium sp. WYCCWR10014
Rhizobium sp. WYCCWR10026
Rhizobium sp. WYCCWR10027
Rhizobium sp. WYCCWR10029
Rhizobium sp. WYCCWR10038
Rhizobium sp. WYCCWR10039
Rhizobium sp. WYCCWR10042
Rhizobium sp. WYCCWR10051
Rhizobium sp. WYCCWR10069
Rhizobium sp. WYCCWR10085
Rhizobium sp. WYCCWR10097
Rhizobium sp. YIC4103
Rhizobium sp. YIC4260
Rhizobium sp. YIC5082
Rhizobium sp.
Rhizobium sphaerophysae
Rhizobium sullae
Rhizobium tibeticum
Rhizobium tropici CIAT 899
Rhizobium tropici
Rhizobium tubonense
Rhizobium tumorigenes
Rhizobium vallis
Rhizobium wenxiniae
Rhizobium wuzhouense
Rhizobium yanglingense
Rhizobium/Agrobacterium group
Rhizobium
Rhizomicrobium electricum
Rhizomicrobium palustre
Rhizophagus clarus
Rhizophagus diaphanus
Rhizophagus irregularis DAOM 181602 = DAOM 197198
Rhizophagus irregularis DAOM 197198w
Rhizophagus irregularis
Rhodanobacter denitrificans
Rhodanobacter sp. FW104-R5
Rhodanobacter sp. FW104-R8
Rhodanobacter sp. FW510-R10
Rhodanobacter sp. FW510-R12
Rhodanobacter sp. FW510-T8
Rhodanobacter sp. L36
Rhodanobacter sp. Root480
Rhodanobacter sp. Root627
Rhodanobacter sp. Soil772
Rhodanobacter thiooxydans
Rhodobacter aestuarii
Rhodobacter amnigenus
Rhodobacter calidifons
Rhodobacter capsulatus B6
Rhodobacter capsulatus DE442
Rhodobacter capsulatus R121
Rhodobacter capsulatus SB 1003
Rhodobacter capsulatus Y262
Rhodobacter capsulatus YW1
Rhodobacter capsulatus YW2
Rhodobacter capsulatus
Rhodobacter maris
Rhodobacter ruber
Rhodobacter sp. AKP1
Rhodobacter sp. AP-10
Rhodobacter sp. BACL10 MAG-120910-bin24
Rhodobacter sp. BACL10 MAG-121220-bin24
Rhodobacter sp. CCBAU 10882
Rhodobacter sp. HX-7-19
Rhodobacter sp. Har01
Rhodobacter sp. JA431
Rhodobacter sp. KR11
Rhodobacter sp. SGA-6-6
Rhodobacter sp. SW2
Rhodobacter sp. TJ_12
Rhodobacter sp.
Rhodobacter tardus
Rhodobacter viridis
Rhodobacter
Rhodobacteraceae bacterium CG17_big_fil_post_rev_8_21_14_2_50_63_15
Rhodobacteraceae bacterium HIMB11
Rhodobacteraceae bacterium HTCC2083
Rhodobacteraceae bacterium MED-G08
Rhodobacteraceae bacterium PARR1
Rhodobacteraceae bacterium TMED111
Rhodobacteraceae bacterium TMED160
Rhodobacteraceae bacterium TMED38
Rhodobacterales bacterium 32-66-7
Rhodobacterales bacterium 32-66-9
Rhodobacterales bacterium 32-67-9
Rhodobacterales bacterium CG15_BIG_FIL_POST_REV_8_21_14_020_59_13
Rhodobacterales bacterium CG18_big_fil_WC_8_21_14_2_50_71_9
Rhodobacterales bacterium CG_4_9_14_3_um_filter_71_31
Rhodobacterales bacterium
Rhodobaculum claviforme
Rhodobium gokarnense
Rhodobium orientis
Rhodoblastus acidophilus
Rhodoblastus sphagnicola
Rhodocista pekingensis
Rhodococcus aetherivorans
Rhodococcus corynebacterioides
Rhodococcus erythropolis group
Rhodococcus erythropolis
Rhodococcus fascians
Rhodococcus globerulus
Rhodococcus jostii
Rhodococcus koreensis
Rhodococcus kroppenstedtii
Rhodococcus kyotonensis
Rhodococcus opacus
Rhodococcus oxybenzonivorans
Rhodococcus phenolicus
Rhodococcus pseudokoreensis
Rhodococcus pyridinivorans
Rhodococcus qingshengii
Rhodococcus rhodochrous J3
Rhodococcus rhodochrous J45
Rhodococcus rhodochrous
Rhodococcus ruber
Rhodococcus sp. 008
Rhodococcus sp. 05-339-2
Rhodococcus sp. 05-340-1
Rhodococcus sp. 05-340-2
Rhodococcus sp. 06-1460-1B
Rhodococcus sp. 06-1474-1B
Rhodococcus sp. 06-235-1A
Rhodococcus sp. 06-412-2B
Rhodococcus sp. 06-412-2C
Rhodococcus sp. 06-418-5
Rhodococcus sp. 06-621-2
Rhodococcus sp. 1163
Rhodococcus sp. 1168
Rhodococcus sp. 14-1411-2a
Rhodococcus sp. 14-2470-1b
Rhodococcus sp. 14-2483-1-2
Rhodococcus sp. 14-2496-1d
Rhodococcus sp. 14C212
Rhodococcus sp. 15-649-2-2
Rhodococcus sp. 1R11
Rhodococcus sp. 3A
Rhodococcus sp. 4CII
Rhodococcus sp. 66b
Rhodococcus sp. 75
Rhodococcus sp. A14
Rhodococcus sp. ABRD24
Rhodococcus sp. ACPA1
Rhodococcus sp. AG1013
Rhodococcus sp. AQ5-07
Rhodococcus sp. ARC_M12
Rhodococcus sp. ARC_M5
Rhodococcus sp. ARC_M6
Rhodococcus sp. B10
Rhodococcus sp. B50
Rhodococcus sp. B7740
Rhodococcus sp. BP22
Rhodococcus sp. CH91
Rhodococcus sp. CX
Rhodococcus sp. DK17
Rhodococcus sp. DMU2021
Rhodococcus sp. EPR-157
Rhodococcus sp. Eu-32
Rhodococcus sp. FXJ9.536
Rhodococcus sp. H-CA8f
Rhodococcus sp. H36-A4
Rhodococcus sp. HM1
Rhodococcus sp. IC4_135
Rhodococcus sp. IEGM 1401
Rhodococcus sp. JS3073
Rhodococcus sp. KB6
Rhodococcus sp. KBS0724
Rhodococcus sp. KBW08
Rhodococcus sp. KRD162
Rhodococcus sp. KRD197
Rhodococcus sp. LB1
Rhodococcus sp. Leaf278
Rhodococcus sp. M8
Rhodococcus sp. MEB064
Rhodococcus sp. MSC1_016
Rhodococcus sp. NCIMB 12038
Rhodococcus sp. NJ-530
Rhodococcus sp. Ni2
Rhodococcus sp. OK302
Rhodococcus sp. P-2
Rhodococcus sp. P1Y
Rhodococcus sp. RDE2
Rhodococcus sp. Rp3
Rhodococcus sp. SBT000017
Rhodococcus sp. SMB37
Rhodococcus sp. UNC23MFCrub1.1
Rhodococcus sp. USK13
Rhodococcus sp. W8901
Rhodococcus sp. WAY2
Rhodococcus sp. WB1
Rhodococcus sp. WB9
Rhodococcus sp. WWJCD1
Rhodococcus sp. WY5
Rhodococcus sp. YH3-3
Rhodococcus sp. Z13
Rhodococcus sp. ZPP
Rhodococcus triatomae
Rhodococcus trifolii
Rhodococcus wratislaviensis
Rhodococcus yananensis
Rhodococcus yunnanensis
Rhodococcus zopfii
Rhodococcus
Rhodocyclaceae bacterium
Rhodocyclales bacterium GT-UBC
Rhodocyclales bacterium
Rhodocyclus gracilis
Rhodocyclus tenuis
Rhodocytophaga rosea
Rhodoferax antarcticus ANT.BR
Rhodoferax antarcticus
Rhodoferax aquaticus
Rhodoferax bucti
Rhodoferax fermentans
Rhodoferax ferrireducens
Rhodoferax koreense
Rhodoferax lacus
Rhodoferax sp. MIZ03
Rhodoferax sp. U11-2br
Rhodoferax
Rhodoflexus caldus
Rhodohalobacter sp. SW132
Rhodomicrobium lacus
Rhodomicrobium sp. Az07
Rhodomicrobium udaipurense JA643
Rhodomicrobium udaipurense
Rhodomicrobium vannielii ATCC 17100
Rhodomicrobium vannielii
Rhodomicrobium
Rhodophyticola porphyridii
Rhodopila globiformis
Rhodopirellula sallentina SM41
Rhodopirellula sallentina
Rhodopirellula sp. JC737
Rhodopirellula sp. MGV
Rhodoplanes elegans
Rhodoplanes piscinae
Rhodoplanes roseus
Rhodoplanes sp. TEM
Rhodoplanes tepidamans
Rhodoplanes
Rhodopseudomonas boonkerdii
Rhodopseudomonas faecalis
Rhodopseudomonas infernalis
Rhodopseudomonas lichen
Rhodopseudomonas oryzae
Rhodopseudomonas palustris BisB5
Rhodopseudomonas palustris CGA009
Rhodopseudomonas palustris HaA2
Rhodopseudomonas palustris TIE-1
Rhodopseudomonas palustris
Rhodopseudomonas pentothenatexigens
Rhodopseudomonas pseudopalustris
Rhodopseudomonas rhenobacensis
Rhodopseudomonas sp. 99D
Rhodopseudomonas sp. AAP120
Rhodopseudomonas sp. B29
Rhodopseudomonas sp. BROC11
Rhodopseudomonas sp. BR0G17
Rhodopseudomonas sp. BROM22
Rhodopseudomonas sp. HMD88
Rhodopseudomonas sp. HMD89
Rhodopseudomonas sp. P2A-2r
Rhodopseudomonas sp. WA056
Rhodopseudomonas thermotolerans
Rhodopseudomonas
Rhodosalinus halophilus
Rhodosalinus sediminis
Rhodospira trueperi
Rhodospirillaceae bacterium BRH_c57
Rhodospirillaceae bacterium LM-1
Rhodospirillaceae bacterium TMED167
Rhodospirillaceae bacterium
Rhodospirillales bacterium 20-60-12
Rhodospirillales bacterium 20-64-7
Rhodospirillales bacterium 47_12_T64
Rhodospirillales bacterium
Rhodospirillum centenum SW
Rhodospirillum centenum
Rhodospirillum rubrum ATCC 11170
Rhodospirillum rubrum F11
Rhodospirillum rubrum
Rhodothalassium salexigens DSM 2132
Rhodothalassium salexigens
Rhodotorula mucilaginosa
Rhodovarius crocodyli
Rhodovastum atsumiense
Rhodovibrio salinarum
Rhodovulum adriaticum
Rhodovulum bhavnagarense
Rhodovulum euryhalinum
Rhodovulum imhoffii
Rhodovulum kholense
Rhodovulum marinum
Rhodovulum robiginosum
Rhodovulum sp. 12E13
Rhodovulum sp. BSW8
Rhodovulum sp. CP-10
Rhodovulum sp. ES.010
Rhodovulum sp. MB263
Rhodovulum sp. NI22
Rhodovulum sp. P5
Rhodovulum sp. PH10
Rhodovulum sp. SN0303
Rhodovulum steppense
Rhodovulum strictum
Rhodovulum sulfidophilum DSM 1374
Rhodovulum sulfidophilum
Rhodovulum tesquicola
Rhodovulum viride
Rhodovulum visakhapatnamense
Rhodovulum
Richelia cyanobiont of Rhizosolenia sp.
Richelia intracellularis HH01
Richelia intracellularis HM01
Richelia intracellularis
Richelia sinica
Richelia sp. SC01
Ricinus communis
Riemerella anatipestifer
Rippkaea orientalis PCC 8801
Rippkaea orientalis
Rivicola pingtungensis
Rivularia sp. IAM M-261
Rivularia sp. PCC 7116
Rivularia sp. UAM 397
Rivularia sp. UAM 414
Robbsia andropogonis
Robbsia sp. Bb-Pol-6
Robertkochia marina
Robertkochia sediminum
Robertmurraya massiliosenegalensis
Robiginitalea biformata
Robiginitalea marina
Robiginitalea myxolifaciens
Robiginitomaculum sp.
Robinsoniella peoriensis
Robinsoniella sp. KNHs210
Robinsoniella sp. RHS
Rodentibacter pneumotropicus
Romboutsia hominis
Romboutsia lituseburensis DSM 797
Romboutsia lituseburensis
Romboutsia sp. 1001713B170131_170501_G6
Romboutsia sp. CE17
Romboutsia sp. Marseille-P6047
Romboutsia
Romeria gracilis
Romeriopsis navalis
Rosa chinensis
Roseateles aquatilis
Roseateles depolymerans
Roseateles sp. BIM B-1768
Roseateles sp. YR242
Roseateles terrae
Roseburia faecis
Roseburia hominis A2-183
Roseburia hominis
Roseburia intestinalis L1-82
Roseburia intestinalis
Roseburia inulinivorans
Roseburia sp. AF02-12
Roseburia sp. BX0805
Roseburia sp. CAG: 100
Roseburia sp. CAG: 18
Roseburia sp. CAG: 18_43_25
Roseburia sp. CAG: 197
Roseburia sp. CAG: 303
Roseburia sp. CAG: 309
Roseburia sp. CAG: 380
Roseburia sp. CAG: 45
Roseburia sp. CAG: 50
Roseburia sp. OM02-15
Roseburia sp. TF10-5
Roseburia
Roseiarcus fermentans
Roseibaca ekhonensis
Roseibacterium beibuensis
Roseibium aggregatum
Roseibium denhamense
Roseibium hamelinense
Roseibium litorale
Roseibium marinum
Roseibium sp. DSM 29163
Roseibium sp. Sym1
Roseibium suaedae
Roseicella frigidaeris
Roseicitreum antarcticum
Roseiconus lacunae
Roseicyclus mahoneyensis
Roseiflexus castenholzii DSM 13941
Roseiflexus castenholzii
Roseiflexus sp. RS-1
Roseiflexus sp.
Roseinatronobacter thiooxidans
Roseisalinus antarcticus
Roseivivax jejudonensis
Roseivivax lentus
Roseivivax marinus
Roseivivax sp. THAF30
Rosenbergiella nectarea
Roseobacter denitrificans OCh 114
Roseobacter denitrificans
Roseobacter litoralis
Roseobacter sp. AzwK-3b
Roseobacter sp. CCS2
Roseobacter sp. H9
Roseobacter sp. OBYS 0001
Roseobacteraceae
Roseofilum reptotaenium
Roseomonas aerilata
Roseomonas aestuarii
Roseomonas genomospecies 6
Roseomonas harenae
Roseomonas ludipueritiae
Roseomonas oleicola
Roseomonas oryzicola
Roseomonas ponticola
Roseomonas rhizosphaerae
Roseomonas sp. 18066
Roseomonas sp. ACRSG
Roseomonas sp. AR75
Roseomonas sp. GC11
Roseomonas sp. JC162
Roseomonas sp. KE2513
Roseomonas sp. MO-31
Roseomonas sp. NAR14
Roseomonas sp. NPKOSM-4
Roseomonas sp. SG15
Roseomonas sp. SSH11
Roseomonas sp. SXEYE001
Roseomonas wenyumeiae
Roseospira goensis
Roseospira marina
Roseospira navarrensis
Roseospira visakhapatnamensis
Roseospirillum parvum
Roseovarius aestuarii
Roseovarius azorensis
Roseovarius confluentis
Roseovarius gaetbuli
Roseovarius marisflavi
Roseovarius mucosus DSM 17069
Roseovarius mucosus
Roseovarius nanhaiticus
Roseovarius sp. 217
Roseovarius sp. A-2
Roseovarius sp. TE539
Roseovarius sp. TM1035
Rossellomorea aquimaris
Rossellomorea marisflavi
Rossellomorea vietnamensis
Rothia aerolata
Rothia mucilaginosa DY-18
Rouxiella aceris
Ruania alkalisoli
Ruania rhizosphaerae
Ruaniaceae bacterium KH17
Rubellimicrobium roseum
Rubellimicrobium rubrum
Rubeoparvulum massiliense
Rubidibacter lacunae KORDI 51-2
Rubinisphaera brasiliensis
Rubinisphaera margarita
Rubinisphaera sp. JC750
Rubneribacter badeniensis
Rubrimonas cliftonensis
Rubripirellula reticaptiva
Rubrivirga marina
Rubrivirga sp. SAORIC476
Rubrivivax albus
Rubrivivax benzoatilyticus JA2 = ATCC BAA-35
Rubrivivax benzoatilyticus
Rubrivivax gelatinosus IL144
Rubrivivax gelatinosus
Rubrivivax sp. A210
Rubrivivax
Rubrobacter aplysinae
Rubrobacter marinus
Rubrobacter radiotolerans
Rudaeicoccus suwonensis
Rudanella paleaurantiibacter
Ruegeria conchae
Ruegeria sp. 6PALISEP08
Ruegeria sp. P4
Rufibacter glacialis
Rufibacter hautae
Rufibacter immobilis
Rufibacter latericius
Rufibacter quisquiliarum
Rufibacter radiotolerans
Rufibacter roseus
Rufibacter ruber
Rufibacter sediminis
Rufibacter sp. DG15C
Rufibacter sp. LB8
Rufibacter sp. SYSU D00344
Rufibacter sp. SYSU D00433
Rufibacter sp. XAAS-G3-1
Rufibacter tibetensis
Ruficoccus amylovorans
Ruficoccus sp. ZRK36
Rugamonas aquatica
Rugamonas rivuli
Rugamonas rubra
Rugamonas sp. CCM 8940
Ruminiclostridium cellobioparum DSM 1351 = ATCC 15832
Ruminiclostridium cellobioparum subsp. termitidis CT1112
Ruminiclostridium cellobioparum
Ruminiclostridium cellulolyticum H10
Ruminiclostridium cellulolyticum
Ruminiclostridium herbifermentans
Ruminiclostridium hungatei
Ruminiclostridium josui
Ruminiclostridium papyrosolvens C7
Ruminiclostridium papyrosolvens DSM 2782
Ruminiclostridium papyrosolvens
Ruminiclostridium sufflavum DSM 19573
Ruminiclostridium sufflavum
Ruminococcaceae bacterium CPB6
Ruminococcaceae bacterium FB2012
Ruminococcaceae bacterium KH2T8
Ruminococcaceae bacterium R-25
Ruminococcaceae bacterium YAD3003
Ruminococcus albus 7 = DSM 20455
Ruminococcus albus 8
Ruminococcus albus SY3
Ruminococcus albus
Ruminococcus bicirculans
Ruminococcus flavefaciens
Ruminococcus obeum CAG: 39
Ruminococcus sp. 1001713B170207_170306_F5
Ruminococcus sp. AF12-5
Ruminococcus sp. AF16-50
Ruminococcus sp. AF17-12
Ruminococcus sp. AF17-1AC
Ruminococcus sp. AF17-22AC
Ruminococcus sp. AF17-24
Ruminococcus sp. AF17-6LB
Ruminococcus sp. AF17-6
Ruminococcus sp. AF18-29
Ruminococcus sp. AF19-15
Ruminococcus sp. AF19-29
Ruminococcus sp. AF19-4LB
Ruminococcus sp. AF20-12LB
Ruminococcus sp. AF21-11
Ruminococcus sp. AF21-42
Ruminococcus sp. AF24-16
Ruminococcus sp. AF25-13
Ruminococcus sp. AF25-17
Ruminococcus sp. AF25-19
Ruminococcus sp. AF25-23LB
Ruminococcus sp. AF25-3LB
Ruminococcus sp. AF26-25AA
Ruminococcus sp. AF27-11AA
Ruminococcus sp. AF27-12AA
Ruminococcus sp. AF27-3
Ruminococcus sp. AF31-14BH
Ruminococcus sp. AF31-8BH
Ruminococcus sp. AF32-2AC
Ruminococcus sp. AF33-11BH
Ruminococcus sp. AF34-12
Ruminococcus sp. AF37-20
Ruminococcus sp. AF41-9
Ruminococcus sp. AF45-4BH
Ruminococcus sp. AF46-10NS
Ruminococcus sp. AM09-18-1
Ruminococcus sp. AM12-48
Ruminococcus sp. AM16-34
Ruminococcus sp. AM22-13
Ruminococcus sp. AM22-14LB
Ruminococcus sp. AM23-1LB
Ruminococcus sp. AM23-1
Ruminococcus sp. AM26-12LB
Ruminococcus sp. AM27-11LB
Ruminococcus sp. AM27-16
Ruminococcus sp. AM27-27
Ruminococcus sp. AM28-13
Ruminococcus sp. AM29-10LB
Ruminococcus sp. AM29-19LB
Ruminococcus sp. AM29-1LB
Ruminococcus sp. AM29-1
Ruminococcus sp. AM29-5AC
Ruminococcus sp. AM31-15AC
Ruminococcus sp. AM32-17LB
Ruminococcus sp. AM33-14
Ruminococcus sp. AM34-9LB
Ruminococcus sp. AM36-2AA
Ruminococcus sp. AM36-5
Ruminococcus sp. AM41-10BH
Ruminococcus sp. AM41-2AC
Ruminococcus sp. AM42-10AC
Ruminococcus sp. AM42-11
Ruminococcus sp. AM43-6
Ruminococcus sp. AM44-9AT
Ruminococcus sp. AM46-18
Ruminococcus sp. AM47-2BH
Ruminococcus sp. AM49-10BH
Ruminococcus sp. AM49-8
Ruminococcus sp. AM54-14NS
Ruminococcus sp. AM54-1NS
Ruminococcus sp. AM57-5
Ruminococcus sp. CAG: 17
Ruminococcus sp. CAG: 353
Ruminococcus sp. CAG: 55
Ruminococcus sp. CAG: 579
Ruminococcus sp. CAG: 57
Ruminococcus sp. CAG: 90
Ruminococcus sp. D55t1_190419_H1
Ruminococcus sp. FC2018
Ruminococcus sp. HUN007
Ruminococcus sp. J1101004_170508_H5
Ruminococcus sp. KGMB03662
Ruminococcus sp. MSJ-25
Ruminococcus sp. NK3A76
Ruminococcus sp. OF02-6
Ruminococcus sp. OF03-6AA
Ruminococcus sp. OF05-2BH
Ruminococcus sp. OM04-4AA
Ruminococcus sp. OM07-17
Ruminococcus sp. OM07-7
Ruminococcus sp. OM08-13AT
Ruminococcus sp. OM08-7
Ruminococcus sp. OM08-9BH
Ruminococcus sp. SR1/5
Ruminococcus sp. TF06-23
Ruminococcus sp. TF08-4
Ruminococcus sp. TF10-12AC
Ruminococcus sp. TF10-6
Ruminococcus sp. TF11-2AC
Ruminococcus sp. TF12-2
Ruminococcus sp. TM463
Ruminococcus sp. YE71
Ruminococcus sp. YE78
Ruminococcus sp.
Ruminococcus
Rummeliibacillus stabekisii
Runella zeae
Ruthenibacterium lactatiformans
SAR116 cluster alpha proteobacterium HIMB100
SAR116 cluster bacterium MED-G04
SAR116 cluster bacterium
SAR92 bacterium BACL16 MAG-120322-bin99
SAR92 bacterium BACL16 MAG-120619-bin48
Sabulibacter ruber
Sabulicella rubraurantiaca
Saccharibacillus sp. O23
Saccharicrinis aurantiacus
Saccharicrinis fermentans DSM 9555 = JCM 21142
Saccharicrinis fermentans
Saccharomonospora piscinae
Saccharomonospora viridis
Saccharophagus degradans 2-40
Saccharophagus degradans
Saccharopolyspora elongata
Saccharopolyspora erythraea D
Saccharopolyspora erythraea NRRL 2338
Saccharopolyspora erythraea
Saccharopolyspora flava
Saccharopolyspora gloriosae
Saccharopolyspora hordei
Saccharopolyspora phatthalungensis
Saccharopolyspora pogona
Saccharopolyspora rectivirgula
Saccharopolyspora rosea
Saccharopolyspora shandongensis
Saccharopolyspora sp. ASAGF58
Saccharopolyspora sp. HNM0983
Saccharopolyspora sp. HNM0986
Saccharopolyspora sp. K220
Saccharopolyspora sp. NFXS83
Saccharopolyspora sp. WRP 15-2
Saccharopolyspora subtropica
Saccharospirillum mangrovi
Saccharospirillum salsuginis
Saccharospirillum
Saccharothrix algeriensis
Saccharothrix australiensis
Saccharothrix carnea
Saccharothrix coeruleofusca
Saccharothrix ecbatanensis
Saccharothrix luteola
Saccharothrix saharensis
Saccharothrix sp. 6-C
Saccharothrix sp. NRRL B-16314
Saccharothrix sp. NRRL B-16348
Saccharothrix sp. S26
Saccharothrix tamanrassetensis
Saccharothrix texasensis
Saccharothrix variisporea
Saccharothrix violaceirubra
Saccharum hybrid cultivar R570
Sagittula salina
Sagittula sp. P11
Sala cibi
Salaquimonas pukyongi
Salegentibacter echinorum
Salegentibacter salegens
Salegentibacter sediminis
Salegentibacter sp. Hel_I_6
Salibacter halophilus
Salibacterium halotolerans
Salidesulfovibrio brasiliensis
Salidesulfovibrio onnuriiensis
Salileptolyngbya diazotrophicum SCSIO 43686
Salimicrobium humidisoli
Salimicrobium salexigens
Salimicrobium
Salinadaptatus halalkaliphilus
Salinibacter ruber
Salinibacterium amurskyense
Salinibacterium sp. M195
Salinibacterium sp. NG253
Salinibacterium sp. PAMC 21357
Salinibacterium sp. SWN1162
Salinibacterium sp. SWN139
Salinibacterium sp. SWN167
Salinibacterium sp. SWN248
Salinibacterium sp. UTAS2018
Salinibacterium xinjiangense
Salinicoccus cyprini
Salinicoccus halitifaciens
Salinicoccus kekensis
Salinicoccus qingdaonensis
Salinicoccus roseus
Salinicoccus sp. ID82-1
Salinigranum halophilum
Salinimicrobium oceani
Salinimonas chungwhensis
Salinimonas iocasae
Salinimonas lutimaris
Salinimonas marina
Salinimonas profundi
Salinimonas sediminis
Salinisphaera sp. QIT1-3
Salinispira pacifica
Salinivibrio socompensis
Salinivirga cyanobacteriivorans
Salipaludibacillus aurantiacus
Salipiger abyssi
Salipiger sp. CCB-MM3
Salipiger sp. P9
Salipiger sp. PrR004
Salipiger thiooxidans
Salisediminibacterium haloalkalitolerans
Salisediminibacterium selenitireducens
Salmonella bongori
Salmonella enterica subsp. arizonae
Salmonella enterica subsp. enterica serovar Enteritidis str. 2010K-0286
Salmonella enterica
Salsuginibacillus kocurii
Sandaracinus amylolyticus
Sandaracinus
Sandarakinorhabdus limnophila
Sarcina sp. DSM 11001
Sarcina ventriculi
Satyrvirus sp.
Scatolibacter rhodanostii
Schaalia odontolytica
Schinkia azotoformans LMG 9581
Schinkia azotoformans MEV 2011
Schinkia azotoformans
Schizothrix sp. UAM 403
Schnuerera ultunensis
Schwartzia succinivorans DSM 10502
Schwartzia succinivorans
Sciscionella marina
Sciscionella sp. SE31
Scytonema bohnerii CG15
Scytonema bohnerii Ind24
Scytonema bohnerii Ind25
Scytonema cf. mirabile ER0515.01
Scytonema foetidum NUACC05
Scytonema foetidum NUACC06
Scytonema hofmannii PCC 7110
Scytonema hofmannii
Scytonema javanicum FACHB-887
Scytonema sp. DC-A
Scytonema sp. FACHB-620
Scytonema sp. FGP-7A
Scytonema sp. HK-05
Scytonema sp. LEGE 07189
Scytonema sp. NC-4B
Scytonema sp. NCC-4B
Scytonema sp. NIES-4073
Scytonema sp. UIC 10036
Scytonema sp.
Scytonema tolypothrichoides VB-61278
Scytonema tolypothrichoides
Scytonemataceae bacterium CCM-UFV057
Sedimenticola selenatireducens
Sedimenticola sp. SB48
Sedimenticola thiotaurini
Sedimentimonas flavescens
Sediminibacillus
Sediminibacterium salmoneum
Sediminibacterium soli
Sediminibacterium sp. RHBRASLY1
Sediminispirochaeta bajacaliforniensis
Sediminispirochaeta smaragdinae DSM 11293
Sediminispirochaeta smaragdinae
Segetibacter sp. 3557_3
Selenihalanaerobacter shriftii
Seleniivibrio woodruffii
Selenomonas artemidis F0399
Selenomonas artemidis
Selenomonas bovis
Selenomonas felix
Selenomonas flueggei ATCC 43531
Selenomonas flueggei
Selenomonas infelix ATCC 43532
Selenomonas infelix
Selenomonas massiliensis
Selenomonas montiformis
Selenomonas noxia ATCC 43541
Selenomonas noxia F0398
Selenomonas noxia
Selenomonas ruminantium subsp. lactilytica TAM6421
Selenomonas ruminantium
Selenomonas sp. AE3005
Selenomonas sp. CM52
Selenomonas sp. F0473
Selenomonas sp. FOBRC6
Selenomonas sp. FOBRC9
Selenomonas sp. ND2010
Selenomonas sp. mPRGC5
Selenomonas sp. oral taxon 126
Selenomonas sp. oral taxon 136
Selenomonas sp. oral taxon 137 str. F0430
Selenomonas sp. oral taxon 138 str. F0429
Selenomonas sp. oral taxon 138
Selenomonas sp. oral taxon 149 str. 67H29BP
Selenomonas sp. oral taxon 149
Selenomonas sp. oral taxon 478
Selenomonas sp. oral taxon 892 str. F0426
Selenomonas sp. oral taxon 892
Selenomonas sp. oral taxon 920
Selenomonas sputigena ATCC 35185
Selenomonas sputigena
Selenomonas timonae
Selenomonas
Senegalimassilia anaerobia
Serendipita vermifera ‘subsp. bescii’
Serinibacter arcticus
Serpentinicella alkaliphila
Serpentinimonas barnesii
Serpentinimonas raichei
Serratia liquefaciens
Serratia marcescens
Serratia proteamaculans
Serratia sp. ATCC 39006
Serratia ureilytica
Sesamum indicum
Sessilibacter corallicola
Sharpea azabuensis
Sharpea porci
Shewanella amazonensis SB2B
Shewanella avicenniae
Shewanella benthica KT99
Shewanella benthica
Shewanella denitrificans OS217
Shewanella dokdonensis
Shewanella frigidimarina
Shewanella hafniensis
Shewanella halifaxensis HAW-EB4
Shewanella loihica PV-4
Shewanella mangrovi
Shewanella oneidensis MR-1
Shewanella oneidensis
Shewanella piezotolerans WP3
Shewanella sp. C32
Shewanella sp. HN-41
Shewanella sp. Pdp11
Shewanella sp. SG41-4
Shewanella sp. SM74
Shewanella sp. W3-18-1
Shewanella sp. cp20
Shewanella violacea DSS12
Shewanella violacea
Shewanella woodyi ATCC 51908
Shewanella yunxiaonensis
Shewanella
Shigella boydii
Shigella flexneri
Shigella sonnei
Shimia aestuarii
Shimia gijangensis
Shimia sediminis
Shimia sp. WX04
Shimia thalassica
Shinella kummerowiae
Shinella sp. WSJ-2
Siccirubricoccus deserti
Siccirubricoccus phaeus
Siculibacillus lacustris
Sideroxyarcus emersonii
Sideroxydans lithotrophicus ES-1
Sideroxydans lithotrophicus
Silanimonas lenta
Silicimonas algicola
Silvanigrella aquatica
Silvibacterium bohemicum
Silvibacterium dinghuense
Simiduia aestuariiviva
Simiduia agarivorans SA1 = DSM 21679
Simiduia agarivorans
Siminovitchia fortis
Simonsiella muelleri ATCC 29453
Solibacillus silvestris
Solidesulfovibrio aerotolerans
Solidesulfovibrio alcoholivorans
Solidesulfovibrio carbinolicus
Solidesulfovibrio carbinoliphilus subsp. oakridgensis
Solidesulfovibrio carbinoliphilus
Solidesulfovibrio fructosivorans JJ
Solidesulfovibrio fructosivorans
Solidesulfovibrio magneticus RS-1
Solidesulfovibrio magneticus str. Maddingley MBC34
Solide sulfovibrio magneticus
Solimonas aquatica
Solimonas marina
Solimonas variicoloris
Solirubrobacter ginsenosidimutans
Solirubrobacter pauli
Solirubrobacter phytolaccae
Solirubrobacter sp. URHD0082
Solirubrobacter taibaiensis
Soonwooa buanensis
Sorangiineae bacterium NIC37A 2
Sorangium cellulosum
Sorangium sp. wiwo2
Sorangium
Sorghum bicolor
Spartobacteria bacterium LR76
Sphaerisporangium album
Sphaerobacter thermophilus
Sphaerochaeta associata
Sphaerochaeta globosa str. Buddy
Sphaerochaeta globosa
Sphaerochaeta pleomorpha str. Grapes
Sphaerochaeta pleomorpha
Sphaerospermopsis aphanizomenoides M17
Sphaerospermopsis aphanizomenoides NRE2
Sphaerospermopsis aphanizomenoides UADFA13
Sphaerospermopsis aphanizomenoides UADFA3
Sphaerospermopsis aphanizomenoides UADFA5
Sphaerospermopsis aphanizomenoides UADFA6
Sphaerospermopsis aphanizomenoides UADFA7
Sphaerospermopsis aphanizomenoides UADFA8
Sphaerospermopsis aphanizomenoides
Sphaerospermopsis kisseleviana NIES-73
Sphaerospermopsis kisseleviana
Sphaerospermopsis reniformis
Sphaerospermopsis sp. FACHB-1194
Sphaerospermopsis sp. LEGE 00249
Sphaerospermopsis sp. LEGE 08334
Sphaerospermopsis torques-reginae
Sphaerospermopsis
Sphaerotilus hippei
Sphaerotilus natans subsp. natans DSM 6575
Sphaerotilus natans
Sphaerotilus sulfidivorans
Sphingobacteriales bacterium 39-40-5
Sphingobacteriia bacterium 35-40-8
Sphingobacterium chungjuense
Sphingobacterium corticibacter
Sphingobacterium deserti
Sphingobacterium faecium PCAi_F2.5
Sphingobacterium griseoflavum
Sphingobacterium multivorum
Sphingobacterium sp. 1.A.4
Sphingobacterium sp. CFCC 11742
Sphingobacterium sp. IITKGP-BTPF85
Sphingobacterium sp. KACC 22765
Sphingobacterium sp. PCS056
Sphingobacterium sp. T2
Sphingobacterium sp. lm-10
Sphingobacterium spiritivorum
Sphingobium baderi LL03
Sphingobium baderi
Sphingobium bisphenolivorans
Sphingobium chungbukense
Sphingobium cupriresistens LL01
Sphingobium cupriresistens
Sphingobium herbicidovorans NBRC 16415
Sphingobium herbicidovorans
Sphingobium indicum BiD32
Sphingobium indicum F2
Sphingobium indicum IP26
Sphingobium indicum
Sphingobium sp. BHU LFT2
Sphingobium sp. C100
Sphingobium sp. DC-2
Sphingobium sp. IP1
Sphingobium sp. MI1205
Sphingobium sp. SYK-6
Sphingobium sp. TKS
Sphingobium xanthum
Sphingobium xenophagum
Sphingobium yanoikuyae
Sphingobium
Sphingomicrobium marinum
Sphingomicrobium sp. B8
Sphingomicrobium sp. O-35
Sphingomonadaceae
Sphingomonadales bacterium 28-64-96
Sphingomonadales bacterium 32-65-25
Sphingomonadales bacterium CG_4_10_14_3_um_filter_58_15
Sphingomonadales
Sphingomonas aerolata
Sphingomonas aliaeris
Sphingomonas aracearum
Sphingomonas azotifigens
Sphingomonas bacterium
Sphingomonas bisphenolicum
Sphingomonas deserti
Sphingomonas dokdonensis
Sphingomonas edaphi
Sphingomonas formosensis
Sphingomonas ginkgonis
Sphingomonas glacialis
Sphingomonas guangdongensis
Sphingomonas hankookensis
Sphingomonas hengshuiensis
Sphingomonas japonica
Sphingomonas jaspsi
Sphingomonas kyeonggiensis
Sphingomonas lutea
Sphingomonas melonis
Sphingomonas morindae
Sphingomonas panacisoli
Sphingomonas paucimobilis
Sphingomonas pituitosa
Sphingomonas pokkalii
Sphingomonas populi
Sphingomonas psychrotolerans
Sphingomonas radiodurans
Sphingomonas rubra
Sphingomonas sabuli
Sphingomonas sanguinis
Sphingomonas sanxanigenens
Sphingomonas segetis
Sphingomonas soli
Sphingomonas sp. IF27F7B
Sphingomonas sp. 28-62-11
Sphingomonas sp. 28-62-20
Sphingomonas sp. 28-63-12
Sphingomonas sp. 28-66-16
Sphingomonas sp. 32-62-10
Sphingomonas sp. 35-24ZXX
Sphingomonas sp. 3F27E9-B
Sphingomonas sp. 7/4-4
Sphingomonas sp. AP4-R1
Sphingomonas sp. Ant H11
Sphingomonas sp. BK235
Sphingomonas sp. BK481
Sphingomonas sp. BN140010
Sphingomonas sp. BR12190
Sphingomonas sp. BR12195
Sphingomonas sp. BR12200
Sphingomonas sp. BR12245
Sphingomonas sp. BR12246
Sphingomonas sp. BR12247
Sphingomonas sp. BR12248
Sphingomonas sp. BR12249
Sphingomonas sp. BR12250
Sphingomonas sp. BR12252
Sphingomonas sp. BR12253
Sphingomonas sp. BR12254
Sphingomonas sp. BR12255
Sphingomonas sp. BR12256
Sphingomonas sp. BR12257
Sphingomonas sp. BR12258
Sphingomonas sp. BR12259
Sphingomonas sp. BR12260
Sphingomonas sp. BR12261
Sphingomonas sp. BR12262
Sphingomonas sp. BR12263
Sphingomonas sp. BR12264
Sphingomonas sp. BT553
Sphingomonas sp. CARO-RG-8B-R24-01
Sphingomonas sp. CCBAU 10879
Sphingomonas sp. CCBAU 10921
Sphingomonas sp. CCH5-D11
Sphingomonas sp. CCH9-F2
Sphingomonas sp. CFBP 13720
Sphingomonas sp. CFBP 13733
Sphingomonas sp. ERG5
Sphingomonas sp. G-3-2-10
Sphingomonas sp. G124
Sphingomonas sp. HDW15A
Sphingomonas sp. HMWF008
Sphingomonas sp. IC4-52
Sphingomonas sp. ID 1715
Sphingomonas sp. JC676
Sphingomonas sp. JXJ CY 53
Sphingomonas sp. KNUC167
Sphingomonas sp. Leaf10
Sphingomonas sp. Leaf22
Sphingomonas sp. Leaf25
Sphingomonas sp. Leaf339
Sphingomonas sp. Leaf33
Sphingomonas sp. Leaf407
Sphingomonas sp. Leaf42
Sphingomonas sp. Leaf4
Sphingomonas sp. Leaf67
Sphingomonas sp. MM-1
Sphingomonas sp. NIBR 02145
Sphingomonas sp. OV641
Sphingomonas sp. PAMC 26605
Sphingomonas sp. QA11
Sphingomonas sp. R647
Sphingomonas sp. RB56-2
Sphingomonas sp. REN5
Sphingomonas sp. RP 10(2022)
Sphingomonas sp. Root241
Sphingomonas sp. S-NIH.Pt15_0812
Sphingomonas sp. S2-65
Sphingomonas sp. S2M10
Sphingomonas sp. S8-608
Sphingomonas sp. SE158
Sphingomonas sp. SE220
Sphingomonas sp. TDK1
Sphingomonas sp. UNC305MFCol5.2
Sphingomonas sp. Y49
Sphingomonas sp. YR710
Sphingomonas spermidinifaciens
Sphingomonas suaedae
Sphingomonas trueperi
Sphingomonas turrisvirgatae
Sphingomonas ursincola
Sphingomonas xinjiangensis
Sphingomonas
Sphingopyxis granuli
Sphingopyxis italica
Sphingopyxis macrogoltabida
Sphingopyxis panaciterrulae
Sphingopyxis soli
Sphingopyxis sp. BSNA05
Sphingopyxis sp. FD7
Sphingopyxis sp. JAI108
Sphingopyxis sp. KK2
Sphingopyxis sp. LC363
Sphingopyxis sp. LC81
Sphingopyxis sp. OAS728
Sphingopyxis sp. OPL5
Sphingopyxis sp. Root1497
Sphingosinicella ginsenosidimutans
Sphingosinicella sp. BN140058
Sphingosinithalassobacter portus
Spinacia oleracea
Spiribacter curvatus
Spiribacter roseus
Spiribacter sp. E85
Spiribacter sp. SSL99
Spiribacter
Spirirestis rafaelensis SRS70
Spirochaeta africana DSM 8902
Spirochaeta africana
Spirochaeta aurantia
Spirochaeta cellobiosiphila
Spirochaeta isovalerica
Spirochaeta lutea
Spirochaeta thermophila DSM 6192
Spirochaeta thermophila DSM 6578
Spirochaeta thermophila
Spirochaetaceae bacterium
Spirochaetae bacterium HGW-Spirochaetae-10
Spirochaetae bacterium HGW-Spirochaetae-4
Spirochaetae bacterium HGW-Spirochaetae-8
Spirochaetae bacterium HGW-Spirochaetae-9
Spirochaetales bacterium
Spirochaetes bacterium DG_61
Spirochaetes bacterium GWC2_52_13
Spirochaetes bacterium GWF1_49_6
Spirochaetes bacterium GWF2_52_7
Spirochaetes bacterium RBG_13_68_11
Spirochaetia bacterium
Spirochaetota bacterium
Spirosoma agri
Spirosoma lacussanchae
Spirosoma montaniterrae
Spirosoma pollinicola
Spirosoma radiotolerans
Spirosoma rhododendri
Spirosoma rigui
Spirosoma sp. RHs26
Spirosoma sp. RP8
Spirosoma utsteinense
Spirulina major
Spirulina sp. CS-785/01
Spirulina subsalsa
Spirulina
Spongiibacter marinus
Spongiibacter sp. IMCC21906
Spongiibacter thalassae
Spongiibacter tropicus
Spongiivirga citrea
Sporobacter termitidis DSM 10068
Sporobacter termitidis
Sporocytophaga myxococcoides
Sporolactobacillus inulinus
Sporolactobacillus shoreicorticis
Sporolactobacillus terrae
Sporolactobacillus vineae
Sporolituus thermophilus DSM 23256
Sporolituus thermophilus
Sporomusa acidovorans DSM 3132
Sporomusa acidovorans
Sporomusa malonica
Sporomusa ovata DSM 2662
Sporomusa ovata
Sporomusa silvacetica DSM 10669
Sporomusa silvacetica
Sporomusa sp. GT1
Sporomusa sp. KB1
Sporomusa sphaeroides DSM 2875
Sporomusa sphaeroides
Sporomusa termitida
Sporomusa
Sporomusaceae bacterium FL31
Sporomusaceae bacterium
Sporomusaceae
Sporosalibacterium faouarense
Sporosarcina globispora
Sporosarcina koreensis
Sporosarcina newyorkensis 2681
Sporosarcina newyorkensis
Sporosarcina pasteurii
Sporosarcina psychrophila
Sporosarcina sp. A15
Sporosarcina sp. BI001-red
Sporosarcina sp. D27
Sporosarcina sp. HYO08
Sporosarcina sp. JAI121
Sporosarcina sp. P13
Sporosarcina sp. P17b
Sporosarcina sp. P18a
Sporosarcina sp. P19
Sporosarcina sp. P1
Sporosarcina sp. P20a
Sporosarcina sp. P26b
Sporosarcina sp. P29
Sporosarcina sp. P2
Sporosarcina sp. P33
Sporosarcina sp. P34
Sporosarcina sp. P3
Sporosarcina sp. P7
Sporosarcina sp. PTS2304
Sporosarcina sp. ZBG7A
Sporosarcina ureae
Sporosarcina
Stackebrandtia nassauensis DSM 44728
Stackebrandtia nassauensis
Stakelama flava
Stakelama pacifica
Stanieria cyanosphaera PCC 7437
Stanieria cyanosphaera
Stanieria sp. NIES-3757
Staphylococcus aureus
Staphylococcus pseudintermedius
Staphylothermus hellenicus DSM 12710
Staphylothermus hellenicus
Staphylothermus marinus F1
Staphylothermus marinus
Stappia albiluteola
Stappia indica
Stappia sp. ES.058
Starkeya novella
Starkeya sp. ORS 1474
Stecheria intestinalis
Stenomitos frigidus
Stenotrophomonas bentonitica
Stenotrophomonas chelatiphaga
Stenotrophomonas cyclobalanopsidis
Stenotrophomonas daejeonensis
Stenotrophomonas ginsengisoli
Stenotrophomonas humi
Stenotrophomonas indicatrix
Stenotrophomonas maltophilia RA8
Stenotrophomonas maltophilia group
Stenotrophomonas maltophilia
Stenotrophomonas nematodicola
Stenotrophomonas nitritireducens
Stenotrophomonas pavanii
Stenotrophomonas rhizophila
Stenotrophomonas sp. 169
Stenotrophomonas sp. 278
Stenotrophomonas sp. 364
Stenotrophomonas sp. 9(2022)
Stenotrophomonas sp. ASS1
Stenotrophomonas sp. B1-1
Stenotrophomonas sp. BIGb0135
Stenotrophomonas sp. CC120222-04
Stenotrophomonas sp. CC120223-11
Stenotrophomonas sp. CC22-02
Stenotrophomonas sp. CFBP 13718
Stenotrophomonas sp. CFBP 13725
Stenotrophomonas sp. ESTM1D_MKCIP4_1
Stenotrophomonas sp. GbtcB23
Stenotrophomonas sp. JAI102
Stenotrophomonas sp. KCTC 12332
Stenotrophomonas sp. LM091
Stenotrophomonas sp. Leaf70
Stenotrophomonas sp. MA5
Stenotrophomonas sp. MH181796
Stenotrophomonas sp. MYb57
Stenotrophomonas sp. PI_27
Stenotrophomonas sp. Ps199
Stenotrophomonas sp. S39
Stenotrophomonas sp. S41
Stenotrophomonas sp. SAM-B
Stenotrophomonas sp. SAU14A_NAIMI4_5
Stenotrophomonas sp. SAU14A_NAIMI4_8
Stenotrophomonas sp. SKA14
Stenotrophomonas sp. SPM
Stenotrophomonas sp. SRS1
Stenotrophomonas sp. Ste96
Stenotrophomonas sp. TN21
Stenotrophomonas sp. WZN-1
Stenotrophomonas sp. YAU14A_MKIMI4_1
Stenotrophomonas sp. YAU14D1_LEIMI4_1
Stenotrophomonas sp. ZAC14A_NAIMI4_1
Stenotrophomonas sp. ZAC14D2_NAIMI4_6
Stenotrophomonas sp. ZAC14D2_NAIMI4_7
Stenotrophomonas sp. gx-44
Stenotrophomonas terrae
Stenotrophomonas
Steroidobacter agaridevorans
Steroidobacter agariperforans
Steroidobacter cummioxidans
Steroidobacter denitrificans
Steroidobacter gossypii
Steroidobacter soli
Sterolibacterium denitrificans
Stigmatella aurantiaca
Stigmatella erecta
Stigmatella hybrida
Stigmatella sp. ncwall
Stigonema anomalum TM01-11
Stigonema anomalum TM01-1
Stigonema anomalum TM01-4
Stigonema anomalum TM01-6
Stigonema dinghuense DHS007
Stigonema elegans WYS5-11
Stigonema elegans WYS5-7
Stigonema fragile E3
Stigonema hainanensis Q17-2
Stigonema hainanensis Q17-5
Stigonema hainanensis 017-8
Stigonema hainanensis Q17
Stigonema hormoides Q19-1
Stigonema hormoides Q19-2
Stigonema hormoides WYS4
Stigonema intermedium YN4
Stigonema intermedium YN5
Stigonema ocellatum Q10-11
Stigonema ocellatum Q10-1
Stigonema ocellatum Q10-2
Stigonema ocellatum Q10-4
Stigonema ocellatum Q10-7
Stigonema ocellatum Q10-9
Stigonema ocellatum Q38-11
Stigonema ocellatum Q38-2
Stigonema ocellatum Q38-3
Stigonema ocellatum Q38-4
Stigonema ocellatum Q38-6
Stigonema ocellatum Q38-8
Stigonema ocellatum SAG 48.90 = DSM 106950
Stigonema panniforme H54-10
Stigonema panniforme H54-1
Stigonema panniforme H54-3
Stigonema panniforme H54-7
Stigonema panniforme H54-9
Stigonema panniforme LS21-4
Stigonema panniforme QYS29-1
Stigonema panniforme QYS29-4
Stigonema panniforme QYS29-5
Stigonema panniforme QYS29-6
Stigonema panniforme ZH17
Stigonema panniforme ZH1
Stigonema parciramosum H34-10
Stigonema parciramosum H34-12
Stigonema sp. LS9-1
Stomatobaculum longum
Streptacidiphilus anmyonensis
Streptacidiphilus griseoplanus
Streptacidiphilus jiangxiensis
Streptacidiphilus melanogenes
Streptacidiphilus neutrinimicus
Streptacidiphilus pinicola
Streptococcus australis
Streptococcus hillyeri
Streptococcus phocae
Streptococcus pneumoniae
Streptococcus pyogenes MGAS15252
Streptococcus pyogenes MGAS1882
Streptococcus suis
Streptomonospora alba
Streptomonospora litoralis
Streptomonospora nanhaiensis
Streptomonospora salina
Streptomonospora sp. PA3
Streptomonospora sp. S1-112
Streptomyces abikoensis
Streptomyces abyssomicinicus
Streptomyces achromogenes
Streptomyces acidicola
Streptomyces acidipaludis
Streptomyces acidiscabies
Streptomyces actinomycinicus
Streptomyces actuosus
Streptomyces adustus
Streptomyces africanus
Streptomyces agglomeratus
Streptomyces akebiae
Streptomyces alanosinicus
Streptomyces albaduncus
Streptomyces albicerus
Streptomyces albidochromogenes
Streptomyces albidoflavus
Streptomyces albidus (ex Kaewkla and Franco 2021)
Streptomyces albiflavescens
Streptomyces albofaciens
Streptomyces alboflavus
Streptomyces alboniger
Streptomyces alboverticillatus
Streptomyces albovinaceus
Streptomyces albus subsp. albus
Streptomyces albus
Streptomyces alfalfae
Streptomyces alkaliterrae
Streptomyces ambofaciens
Streptomyces amritsarensis
Streptomyces anandii
Streptomyces angustmyceticus
Streptomyces anthocyanicus group
Streptomyces anthocyanicus
Streptomyces antibioticus
Streptomyces antimycoticus
Streptomyces antioxidans
Streptomyces anulatus
Streptomyces apocyni
Streptomyces apricus
Streptomyces aquilus
Streptomyces arboris
Streptomyces ardesiacus
Streptomyces armeniacus
Streptomyces asiaticus
Streptomyces asoensis
Streptomyces atratus
Streptomyces atriruber
Streptomyces atroolivaceus
Streptomyces aurantiacus JA 4570
Streptomyces aurantiacus
Streptomyces aurantiogriseus
Streptomyces auratus
Streptomyces aureocirculatus
Streptomyces aureoverticillatus
Streptomyces aureus
Streptomyces autolyticus
Streptomyces avermitilis MA-4680 = NBRC 14893
Streptomyces avermitilis
Streptomyces avidinii
Streptomyces azureus
Streptomyces baarnensis
Streptomyces badius
Streptomyces bambusae
Streptomyces barringtoniae
Streptomyces bathyalis
Streptomyces bauhiniae
Streptomyces beijiangensis
Streptomyces bellus
Streptomyces benahoarensis
Streptomyces bicolor
Streptomyces blattellae
Streptomyces bluensis
Streptomyces bobili
Streptomyces bohaiensis
Streptomyces boluensis
Streptomyces botrytidirepellens
Streptomyces brasiliensis
Streptomyces broussonetiae
Streptomyces buecherae
Streptomyces bungoensis
Streptomyces caatingaensis
Streptomyces cacaoi
Streptomyces caelestis
Streptomyces caeruleatus
Streptomyces cahuitamycinicus
Streptomyces californicus
Streptomyces calvus
Streptomyces canarius
Streptomyces candidus
Streptomyces caniferus
Streptomyces caniscabiei
Streptomyces canus
Streptomyces capitiformicae
Streptomyces capoamus
Streptomyces carpinensis
Streptomyces catenulae
Streptomyces cavernae
Streptomyces cavourensis
Streptomyces cellostaticus
Streptomyces cellulosae
Streptomyces chartreusis
Streptomyces chattanoogensis
Streptomyces chilikensis
Streptomyces chrestomyceticus
Streptomyces chromofuscus
Streptomyces chryseus
Streptomyces cinerochromogenes
Streptomyces cinnabarinus
Streptomyces cinnamoneus
Streptomyces cirratus
Streptomyces clavuligerus
Streptomyces cocklensis
Streptomyces coelicoflavus
Streptomyces coelicolor A3(2)
Streptomyces coeruleorubidus
Streptomyces coffeae
Streptomyces collinus
Streptomyces corallincola
Streptomyces corchorusii
Streptomyces coriariae
Streptomyces coryli
Streptomyces corynorhini
Streptomyces cupreus
Streptomyces curacoi
Streptomyces cyaneofuscatus
Streptomyces cyaneogriseus
Streptomyces cyaneus
Streptomyces cyanogenus
Streptomyces daghestanicus
Streptomyces dangxiongensis
Streptomyces dagingensis
Streptomyces davaonensis
Streptomyces deccanensis
Streptomyces decoyicus
Streptomyces diastaticus
Streptomyces diastatochromogenes
Streptomyces dioscori
Streptomyces djakartensis
Streptomyces drozdowiczii
Streptomyces durbertensis
Streptomyces durhamensis
Streptomyces durmitorensis
Streptomyces durocortorensis
Streptomyces dysideae
Streptomyces echinatus
Streptomyces echinoruber
Streptomyces endocoffeicus
Streptomyces erythrochromogenes
Streptomyces eurocidicus
Streptomyces europaeiscabiei
Streptomyces exfoliatus
Streptomyces fagopyri
Streptomyces ferrugineus
Streptomyces ficellus
Streptomyces filamentosus
Streptomyces filipinensis
Streptomyces finlayi
Streptomyces flaveolus
Streptomyces flaveus
Streptomyces flavidovirens
Streptomyces flavochromogenes
Streptomyces flavofungini
Streptomyces flavotricini
Streptomyces fodineus
Streptomyces formicae
Streptomyces fradiae
Streptomyces fragilis
Streptomyces fructofermentans
Streptomyces fulvorobeus
Streptomyces fulvoviolaceus
Streptomyces fumanus
Streptomyces fumigatiscleroticus
Streptomyces fungicidicus
Streptomyces fuscichromogenes
Streptomyces galbus
Streptomyces galilaeus
Streptomyces gancidicus BKS 13-15
Streptomyces gardneri
Streptomyces gelaticus
Streptomyces genisteinicus
Streptomyces geranii
Streptomyces gilvifuscus
Streptomyces glaucescens
Streptomyces glebosus
Streptomyces globisporus
Streptomyces globosus
Streptomyces glomeratus
Streptomyces goshikiensis
Streptomyces graminilatus
Streptomyces griseicoloratus
Streptomyces griseiscabiei
Streptomyces griseoaurantiacus
Streptomyces griseocarneus
Streptomyces griseochromogenes
Streptomyces griseoflavus
Streptomyces griseofuscus
Streptomyces griseolus
Streptomyces griseoluteus
Streptomyces griseoruber
Streptomyces griseorubiginosus
Streptomyces griseoviridis
Streptomyces griseus
Streptomyces guryensis
Streptomyces harenosi
Streptomyces hawaiiensis
Streptomyces heilongjiangensis
Streptomyces himalayensis
Streptomyces himastatinicus
Streptomyces hirsutus
Streptomyces hokutonensis
Streptomyces humi
Streptomyces humicola
Streptomyces humidus
Streptomyces hundungensis
Streptomyces hyaluromycini
Streptomyces hyderabadensis
Streptomyces hydrogenans
Streptomyces hygroscopicus
Streptomyces iakyrus
Streptomyces iconiensis
Streptomyces incarnatus
Streptomyces indiaensis
Streptomyces indicus
Streptomyces inhibens
Streptomyces inusitatus
Streptomyces ipomoeae
Streptomyces iranensis
Streptomyces janthinus
Streptomyces javensis
Streptomyces jeddahensis
Streptomyces jietaisiensis
Streptomyces jumonjinensis
Streptomyces justiciae
Streptomyces kanamyceticus
Streptomyces kanasensis
Streptomyces kaniharaensis
Streptomyces kasugaensis
Streptomyces katrae
Streptomyces katsurahamanus
Streptomyces kebangsaanensis
Streptomyces klenkii
Streptomyces koyangensis
Streptomyces kronopolitis
Streptomyces kurssanovii
Streptomyces lacrimifluminis
Streptomyces laculatispora
Streptomyces lanatus
Streptomyces lasalocidi
Streptomyces lasiicapitis
Streptomyces lateritius
Streptomyces laurentii
Streptomyces lavendofoliae
Streptomyces lavendulae
Streptomyces lavenduligriseus
Streptomyces leeuwenhoekii
Streptomyces liangshanensis
Streptomyces libani
Streptomyces lienomycini
Streptomyces liliifuscus
Streptomyces liliiviolaceus
Streptomyces lincolnensis
Streptomyces lomondensis
Streptomyces lonarensis
Streptomyces longisporoflavus
Streptomyces longispororuber
Streptomyces longwoodensis
Streptomyces lucensis
Streptomyces lunaelactis
Streptomyces luridiscabiei
Streptomyces lushanensis
Streptomyces lusitanus
Streptomyces luteocolor
Streptomyces luteogriseus
Streptomyces luteolifulvus
Streptomyces lute overticillatus
Streptomyces lydicamycinicus
Streptomyces lydicus
Streptomyces malaysiense
Streptomyces malaysiensis
Streptomyces mangrovisoli
Streptomyces marianii
Streptomyces marincola
Streptomyces marokkonensis
Streptomyces mashuensis
Streptomyces massasporeus
Streptomyces melanogenes
Streptomyces melanosporofaciens
Streptomyces mesophilus
Streptomyces microflavus subgroup
Streptomyces microflavus
Streptomyces milbemycinicus
Streptomyces minutiscleroticus
Streptomyces mirabilis
Streptomyces misionensis
Streptomyces monashensis
Streptomyces monomycini
Streptomyces montanus
Streptomyces morookaense
Streptomyces muensis
Streptomyces murinus
Streptomyces musisoli
Streptomyces mutabilis
Streptomyces mutomycini
Streptomyces naganishii
Streptomyces nanshensis
Streptomyces narbonensis
Streptomyces nashvillensis
Streptomyces netropsis
Streptomyces neyagawaensis
Streptomyces niger
Streptomyces nigra
Streptomyces nigrescens
Streptomyces nitrosporeus
Streptomyces niveiscabiei
Streptomyces niveoruber
Streptomyces niveus NCIMB 11891
Streptomyces niveus
Streptomyces nodosus
Streptomyces nojiriensis
Streptomyces noursei CCRC 11814
Streptomyces noursei PD-1
Streptomyces noursei ZPM
Streptomyces noursei
Streptomyces nymphaeiformis
Streptomyces ochraceiscleroticus
Streptomyces odonnellii
Streptomyces olvivaceoviridis
Streptomyces olivaceus
Streptomyces olivochromogenes
Streptomyces olivoreticuli
Streptomyces olivoverticillatus
Streptomyces pacificus
Streptomyces pactum
Streptomyces palmae
Streptomyces paludis
Streptomyces panaciradicis
Streptomyces paradoxus
Streptomyces parmotrematis
Streptomyces paromomycinus
Streptomyces parvulus
Streptomyces parvus
Streptomyces peucetius subsp. caesius ATCC 27952
Streptomyces peucetius
Streptomyces phaeochromogenes
Streptomyces phaeocafiens
Streptomyces phaeolivaceua
Streptomyces phaeoluteigrieseus
Streptomyces pharetrae CZA14
Streptomyces phyllanthi
Streptomyces pilosus
Streptomyces pinistramenti
Streptomyces platensis
Streptomyces pluripotens
Streptomyces polyasparticus
Streptomyces populi
Streptomyces poriferorum
Streptomyces pratensis
Streptomyces pristinaespiralis
Streptomyces prunicolor
Streptomyces pseudogriseolus
Streptomyces puniciscabiei
Streptomyces purpurascens
Streptomyces purpureus
Streptomyces purpurogeneiscleroticus
Streptomyces qaidamensis
Streptomyces qinzhouensis
Streptomyces radicis
Streptomyces radiopugnans
Streptomyces rapamycinicus NRRL 5491
Streptomyces rapamycinicus
Streptomyces recifensis
Streptomyces regalis
Streptomyces resistomycificus
Streptomyces rhizoryzae
Streptomyces rhizosphaericola
Streptomyces rhizosphaericus
Streptomyces rhizosphaerihabitans
Streptomyces rimosus subsp. pseudoverticillatus
Streptomyces rimosus subsp. rimosus
Streptomyces rimosus
Streptomyces rishiriensis
Streptomyces rochei
Streptomyces roseirectus
Streptomyces roseochromogenus
Streptomyces roseolus
Streptomyces roseoverticillatus
Streptomyces roseus
Streptomyces rubellomurinus
Streptomyces ruber
Streptomyces rubiginosohelvolus
Streptomyces rubradiris
Streptomyces rubrisoli
Streptomyces rubrogriseus
Streptomyces rubrolavendulae
Streptomyces sabulosicollis
Streptomyces sampsonii
Streptomyces samsunensis
Streptomyces scabichelini
Streptomyces scabiei
Streptomyces scopuliridis RB72
Streptomyces scopuliridis
Streptomyces sennicomposti
Streptomyces seoulensis
Streptomyces shenzhenensis
Streptomyces showdoensis
Streptomyces silaceus
Streptomyces silvensis
Streptomyces sindenensis
Streptomyces sioyaensis
Streptomyces smaragdinus
Streptomyces solaniscabiei
Streptomyces solincola
Streptomyces somaliensis
Streptomyces sp. 09ZI22
Streptomyces sp. 1-11
Streptomyces sp. 11-1-2
Streptomyces sp. 13-12-16
Streptomyces sp. 135
Streptomyces sp. 142MFCol3.1
Streptomyces sp. 15-116A
Streptomyces sp. 196(2019)
Streptomyces sp. 2112.2
Streptomyces sp. 2112.3
Streptomyces sp. 2131.1
Streptomyces sp. 2132.2
Streptomyces sp. 2224.1
Streptomyces sp. 2231.1
Streptomyces sp. 2314.4
Streptomyces sp. 2323.1
Streptomyces sp. 2333.5
Streptomyces sp. 2R
Streptomyces sp. 303MFCol5.2
Streptomyces sp. 3211.6
Streptomyces sp. 3211
Streptomyces sp. 3213.3
Streptomyces sp. 2414.6
Streptomyces sp. 351MFTsu5.1
Streptomyces sp. 35G-GA-8
Streptomyces sp. 404i
Streptomyces sp. 43Y-GA-1
Streptomyces sp. 4503
Streptomyces sp. 4F
Streptomyces sp. 5-10
Streptomyces sp. 6-11-2
Streptomyces sp. 769
Streptomyces sp. 7R015
Streptomyces sp. 7R016
Streptomyces sp. 840.1
Streptomyces sp. 8L
Streptomyces sp. 8P21H-1
Streptomyces sp. 8ZJF_21
Streptomyces sp. 9-7
Streptomyces sp. A012304
Streptomyces sp. A0592
Streptomyces sp. A0958
Streptomyces sp. A1-5
Streptomyces sp. A108
Streptomyces sp. A1136
Streptomyces sp. A1277
Streptomyces sp. A13(2022)
Streptomyces sp. A1499
Streptomyces sp. A1547
Streptomyces sp. A15ISP2-DRY2
Streptomyces sp. A2-16
Streptomyces sp. A244
Streptomyces sp. A3M-1-3
Streptomyces sp. AC04842
Streptomyces sp. AC512_CC834
Streptomyces sp. AC550_RSS872
Streptomyces sp. AC555_RSS877
Streptomyces sp. AC558_RSS880
Streptomyces sp. AC602_WCS936
Streptomyces sp. AC627_RSS907
Streptomyces sp. AD681
Streptomyces sp. ADI91-18
Streptomyces sp. ADI92-24
Streptomyces sp. ADI93-02
Streptomyces sp. ADI95-17
Streptomyces sp. ADI96-02
Streptomyces sp. ADI96-15
Streptomyces sp. ADI98-10
Streptomyces sp. ADI98-12
Streptomyces sp. AJS327
Streptomyces sp. AK010
Streptomyces sp. AN091965
Streptomyces sp. AP-93
Streptomyces sp. APSN-46.1
Streptomyces sp. AS02
Streptomyces sp. AcE210
Streptomyces sp. AcH 505
Streptomyces sp. Act143
Streptomyces sp. ActVer
Streptomyces sp. Ag109_G2-15
Streptomyces sp. Ag109_O5-10
Streptomyces sp. Ag109_O5-1
Streptomyces sp. Ag82_G6-1
Streptomyces sp. Ag82_01-15
Streptomyces sp. Akac8
Streptomyces sp. Alain-F2R5
Streptomyces sp. Amel2xC10
Streptomyces sp. B27
Streptomyces sp. B93
Streptomyces sp. BA2
Streptomyces sp. BF-3
Streptomyces sp. BG9H
Streptomyces sp. BH-MK-02
Streptomyces sp. BHT-5-2
Streptomyces sp. BI87
Streptomyces sp. BK022
Streptomyces sp. BK205
Streptomyces sp. BK208
Streptomyces sp. BK239
Streptomyces sp. BK340
Streptomyces sp. BPSDS2
Streptomyces sp. BR123
Streptomyces sp. BRB081
Streptomyces sp. BSE6.1
Streptomyces sp. BSP1
Streptomyces sp. BV129
Streptomyces sp. BV286
Streptomyces sp. BV333
Streptomyces sp. BoleA5
Streptomyces sp. C3-3
Streptomyces sp. CA-210063
Streptomyces sp. CAI 127
Streptomyces sp. CAI-24
Streptomyces sp. CAI-85
Streptomyces sp. CB00072
Streptomyces sp. CB01201
Streptomyces sp. CB01249
Streptomyces sp. CB01373
Streptomyces sp. CB01580
Streptomyces sp. CB01635
Streptomyces sp. CB01881
Streptomyces sp. CB01883
Streptomyces sp. CB02009
Streptomyces sp. CB02056
Streptomyces sp. CB02115
Streptomyces sp. CB02130
Streptomyces sp. CB02261
Streptomyces sp. CB02400
Streptomyces sp. CB02414
Streptomyces sp. CB02460
Streptomyces sp. CB02488
Streptomyces sp. CB02613
Streptomyces sp. CB02923
Streptomyces sp. CB02959
Streptomyces sp. CB02980
Streptomyces sp. CB03234
Streptomyces sp. CB03238
Streptomyces sp. CB09001
Streptomyces sp. CB09030
Streptomyces sp. CBMA123
Streptomyces sp. CBMA152
Streptomyces sp. CBMA29
Streptomyces sp. CBMAI 2042
Streptomyces sp. CC210A
Streptomyces sp. CC228A
Streptomyces sp. CC71
Streptomyces sp. CCM_MD2014
Streptomyces sp. CEV 2-1
Streptomyces sp. CFMR 7
Streptomyces sp. CG 926
Streptomyces sp. CGMCC 4.1796
Streptomyces sp. CL12-4
Streptomyces sp. CMB-StM0423
Streptomyces sp. CME 23
Streptomyces sp. CMSTAAHL-2
Streptomyces sp. CNS654
Streptomyces sp. CS-7
Streptomyces sp. CS014
Streptomyces sp. CS057
Streptomyces sp. CS065A
Streptomyces sp. CS090A
Streptomyces sp. CS113
Streptomyces sp. CS131
Streptomyces sp. CS149
Streptomyces sp. CS159
Streptomyces sp. CS207
Streptomyces sp. CS227
Streptomyces sp. CWNU-1
Streptomyces sp. C
Streptomyces sp. Caat 7-52
Streptomyces sp. CdTB01
Streptomyces sp. CoH17
Streptomyces sp. CoH27
Streptomyces sp. CoT10
Streptomyces sp. D2-8
Streptomyces sp. DASNCL29
Streptomyces sp. DH-12
Streptomyces sp. DH12
Streptomyces sp. DI166
Streptomyces sp. DSM 110735
Streptomyces sp. DSM 15324
Streptomyces sp. DSM 40750
Streptomyces sp. DvalAA-14
Streptomyces sp. DvalAA-19
Streptomyces sp. DvalAA-43
Streptomyces sp. E2N166
Streptomyces sp. E5N91
Streptomyces sp. EAG2
Streptomyces sp. EMB24
Streptomyces sp. EN16
Streptomyces sp. EN23
Streptomyces sp. EN27
Streptomyces sp. ET3-23
Streptomyces sp. F-3
Streptomyces sp. F001
Streptomyces sp. F63
Streptomyces sp. FB2
Streptomyces sp. FH025
Streptomyces sp. FIT100
Streptomyces sp. FM008
Streptomyces sp. FT05W
Streptomyces sp. FXJ1.172
Streptomyces sp. FXJ7.023
Streptomyces sp. FxanaD5
Streptomyces sp. G11C
Streptomyces sp. G2
Streptomyces sp. G3
Streptomyces sp. G44
Streptomyces sp. GBA 94-10 4N24
Streptomyces sp. GC420
Streptomyces sp. GESEQ-13
Streptomyces sp. GESEQ-35
Streptomyces sp. GESEQ-4
Streptomyces sp. GKU 895
Streptomyces sp. GMR22
Streptomyces sp. GMY02
Streptomyces sp. GQFP
Streptomyces sp. GXMU-J5
Streptomyces sp. GZWMJZ-114
Streptomyces sp. GbtcB6
Streptomyces sp. GbtcB7
Streptomyces sp. Go-475
Streptomyces sp. Go40/10
Streptomyces sp. H23
Streptomyces sp. H27-G5
Streptomyces sp. H27-H1
Streptomyces sp. H27-H5
Streptomyces sp. H27-S2
Streptomyces sp. H28
Streptomyces sp. H34-S4
Streptomyces sp. HB-N217
Streptomyces sp. HB132
Streptomyces sp. HB202
Streptomyces sp. HB2AG
Streptomyces sp. HCCB10043
Streptomyces sp. HD
Streptomyces sp. HF10
Streptomyces sp. HGB0020
Streptomyces sp. HL-66
Streptomyces sp. HM190
Streptomyces sp. HNA39
Streptomyces sp. HNM0140
Streptomyces sp. HNM0575
Streptomyces sp. HSG2
Streptomyces sp. HUAS 13-4
Streptomyces sp. HUAS 15-9
Streptomyces sp. HUAS 2-6
Streptomyces sp. HUAS 31
Streptomyces sp. HUCO-GS316
Streptomyces sp. 15
Streptomyces sp. IB2014 016-6
Streptomyces sp. IB201691-2A2
Streptomyces sp. IBSBF 2807
Streptomyces sp. ICBB 8177
Streptomyces sp. ICN441
Streptomyces sp. ICN988
Streptomyces sp. ID38640
Streptomyces sp. IMTB 2501
Streptomyces sp. ISID311
Streptomyces sp. ISL-100
Streptomyces sp. ISL-10
Streptomyces sp. ISL-12
Streptomyces sp. ISL-1
Streptomyces sp. ISL-43
Streptomyces sp. ISL-44
Streptomyces sp. ISL-66
Streptomyces sp. ISL-86
Streptomyces sp. ISL-94
Streptomyces sp. ISL-96
Streptomyces sp. ISL-98
Streptomyces sp. ISL-99
Streptomyces sp. JB150
Streptomyces sp. JHA19
Streptomyces sp. JHA26
Streptomyces sp. JS01
Streptomyces sp. JV178
Streptomyces sp. Je 1-369
Streptomyces sp. Je 1-79
Streptomyces sp. Jing01
Streptomyces sp. KAI 90
Streptomyces sp. KCTC 0041BP
Streptomyces sp. KE1
Streptomyces sp. KM273126
Streptomyces sp. KMM 9044
Streptomyces sp. KO7888
Streptomyces sp. KPB2
Streptomyces sp. KS 21
Streptomyces sp. KS_5
Streptomyces sp. KY70
Streptomyces sp. KhCrAH-43
Streptomyces sp. L-9-10
Streptomyces sp. LBUM 1476
Streptomyces sp. LP11
Streptomyces sp. LRa12
Streptomyces sp. LS1784
Streptomyces sp. LUP30
Streptomyces sp. LUP47B
Streptomyces sp. LaPpAH-108
Streptomyces sp. LcepLS
Streptomyces sp. Lzd4kr
Streptomyces sp. M10
Streptomyces sp. M2CJ-2
Streptomyces sp. M41(2017)
Streptomyces sp. M56
Streptomyces sp. M7
Streptomyces sp. M92
Streptomyces sp. MA3_2.13
Streptomyces sp. MA5143a
Streptomyces sp. MBT33
Streptomyces sp. MBT42
Streptomyces sp. MBT49
Streptomyces sp. MBT51
Streptomyces sp. MBT53
Streptomyces sp. MBT58
Streptomyces sp. MBT60
Streptomyces sp. MBT62
Streptomyces sp. MBT65
Streptomyces sp. MBT84
Streptomyces sp. MBT97
Streptomyces sp. MBT98
Streptomyces sp. MC1
Streptomyces sp. MCA2
Streptomyces sp. MH60
Streptomyces sp. MJM1172
Streptomyces sp. MJM8645
Streptomyces sp. MK37H
Streptomyces sp. MMBL 11-1
Streptomyces sp. MMG1121
Streptomyces sp. MMG1533
Streptomyces sp. MNP-20
Streptomyces sp. MNU76
Streptomyces sp. MNU77
Streptomyces sp. MOE7
Streptomyces sp. MSC1_001
Streptomyces sp. MUM 203J
Streptomyces sp. MUSC 14
Streptomyces sp. MW-W600-10
Streptomyces sp. MZ04
Streptomyces sp. McG3
Streptomyces sp. McG5
Streptomyces sp. McG6
Streptomyces sp. McG8
Streptomyces sp. Mg1
Streptomyces sp. MnatMP-M17
Streptomyces sp. MnatMP-M27
Streptomyces sp. N35
Streptomyces sp. NA02536
Streptomyces sp. NA02950
Streptomyces sp. NA03103
Streptomyces sp. NA13
Streptomyces sp. NBC_00006
Streptomyces sp. NBC_00038
Streptomyces sp. NBC_00059
Streptomyces sp. NBC_00063
Streptomyces sp. NBC_00078
Streptomyces sp. NBC_00083
Streptomyces sp. NBC_00086
Streptomyces sp. NBC_00091
Streptomyces sp. NBC_00094
Streptomyces sp. NBC_00102
Streptomyces sp. NBC_00103
Streptomyces sp. NBC_00120
Streptomyces sp. NBC_00124
Streptomyces sp. NBC_00140
Streptomyces sp. NBC_00154
Streptomyces sp. NBC_00160
Streptomyces sp. NBC_00162
Streptomyces sp. NBC_00183
Streptomyces sp. NBC_00198
Streptomyces sp. NBC_00199
Streptomyces sp. NBC_00233
Streptomyces sp. NBC_00249
Streptomyces sp. NBC_00268
Streptomyces sp. NBC_00320
Streptomyces sp. NBC_00338
Streptomyces sp. NBC_00340
Streptomyces sp. NBC_00347
Streptomyces sp. NBC_00365
Streptomyces sp. NBC_00378
Streptomyces sp. NBC_00401
Streptomyces sp. NBC_00424
Streptomyces sp. NBC_00439
Streptomyces sp. NBC_00452
Streptomyces sp. NBC_00474
Streptomyces sp. NBC_00526
Streptomyces sp. NBC_00555
Streptomyces sp. NBC_00568
Streptomyces sp. NBC_00572
Streptomyces sp. NBC_00620
Streptomyces sp. NBC_00638
Streptomyces sp. NBC_00654
Streptomyces sp. NBC_00687
Streptomyces sp. NBC_00760
Streptomyces sp. NBC_00765
Streptomyces sp. NBC_00809
Streptomyces sp. NBC_00847
Streptomyces sp. NBC_00878
Streptomyces sp. NBC_00893
Streptomyces sp. NBC_00925
Streptomyces sp. NBC_01016
Streptomyces sp. NBC_01142
Streptomyces sp. NBC_01214
Streptomyces sp. NBC_01239
Streptomyces sp. NBC_01264
Streptomyces sp. NBC_01275
Streptomyces sp. NBC_01285
Streptomyces sp. NBC_01306
Streptomyces sp. NBC_01363
Streptomyces sp. NBC_01373
Streptomyces sp. NBC_01381
Streptomyces sp. NBC_01408
Streptomyces sp. NBC_01433
Streptomyces sp. NBC_01443
Streptomyces sp. NBC_01446
Streptomyces sp. NBC_01481
Streptomyces sp. NBC_01500
Streptomyces sp. NBC_01549
Streptomyces sp. NBC_01551
Streptomyces sp. NBC_01565
Streptomyces sp. NBC_01571
Streptomyces sp. NBC_01619
Streptomyces sp. NBC_01669
Streptomyces sp. NBC_01764
Streptomyces sp. NBC_01789
Streptomyces sp. NBRC 110028
Streptomyces sp. NBRC 110030
Streptomyces sp. NBRC 110465
Streptomyces sp. NBRC 110468
Streptomyces sp. NBS 14/10
Streptomyces sp. NBU3104
Streptomyces sp. NE5-10
Streptomyces sp. NEAU-383
Streptomyces sp. NEAU-H22
Streptomyces sp. NEAU-S7GS2
Streptomyces sp. NEAU-W12
Streptomyces sp. NEAU-Y11
Streptomyces sp. NEAU-YJ-81
Streptomyces sp. NK08204
Streptomyces sp. NK15101
Streptomyces sp. NL15-2K
Streptomyces sp. NP-1717
Streptomyces sp. NRRL B-1140
Streptomyces sp. NRRL B-1347
Streptomyces sp. NRRL B-1568
Streptomyces sp. NRRL B-24085
Streptomyces sp. NRRL B-24484
Streptomyces sp. NRRL B-24572
Streptomyces sp. NRRL B-24720
Streptomyces sp. NRRL B-3229
Streptomyces sp. NRRL B-3253
Streptomyces sp. NRRL B-3648
Streptomyces sp. NRRL F-2580
Streptomyces sp. NRRL F-2664
Streptomyces sp. NRRL F-2747
Streptomyces sp. NRRL F-2799
Streptomyces sp. NRRL F-3273
Streptomyces sp. NRRL F-4489
Streptomyces sp. NRRL F-5065
Streptomyces sp. NRRL F-5122
Streptomyces sp. NRRL F-5123
Streptomyces sp. NRRL F-5135
Streptomyces sp. NRRL F-5193
Streptomyces sp. NRRL F-525
Streptomyces sp. NRRL F-5630
Streptomyces sp. NRRL F-5650
Streptomyces sp. NRRL F-5727
Streptomyces sp. NRRL F-5755
Streptomyces sp. NRRL F-5917
Streptomyces sp. NRRL F-6677
Streptomyces sp. NRRL S-1022
Streptomyces sp. NRRL S-118
Streptomyces sp. NRRL S-1448
Streptomyces sp. NRRL S-146
Streptomyces sp. NRRL S-1521
Streptomyces sp. NRRL S-15
Streptomyces sp. NRRL S-1813
Streptomyces sp. NRRL S-1824
Streptomyces sp. NRRL S-1896
Streptomyces sp. NRRL S-237
Streptomyces sp. NRRL S-241
Streptomyces sp. NRRL S-244
Streptomyces sp. NRRL S-31
Streptomyces sp. NRRL S-337
Streptomyces sp. NRRL S-340
Streptomyces sp. NRRL S-350
Streptomyces sp. NRRL S-378
Streptomyces sp. NRRL S-37
Streptomyces sp. NRRL S-475
Streptomyces sp. NRRL S-481
Streptomyces sp. NRRL S-495
Streptomyces sp. NRRL S-4
Streptomyces sp. NRRL S-623
Streptomyces sp. NRRL S-646
Streptomyces sp. NRRL S-87
Streptomyces sp. NRRL S-920
Streptomyces sp. NRRL WC-3549
Streptomyces sp. NRRL WC-3618
Streptomyces sp. NRRL WC-3626
Streptomyces sp. NRRL WC-3701
Streptomyces sp. NRRL WC-3723
Streptomyces sp. NRRL WC-3742
Streptomyces sp. NRRL WC-3744
Streptomyces sp. NRRL WC-3795
Streptomyces sp. NTH33
Streptomyces sp. NTK 937
Streptomyces sp. NWU339
Streptomyces sp. NWU49
Streptomyces sp. Ncost-T10-10d
Streptomyces sp. Ncost-T6T-1
Streptomyces sp. ODS25
Streptomyces sp. OM5714
Streptomyces sp. OUCMDZ-4982
Streptomyces sp. OV198
Streptomyces sp. OspMP-M43
Streptomyces sp. OspMP-M45
Streptomyces sp. P3
Streptomyces sp. PAN_FS17
Streptomyces sp. PBH53
Streptomyces sp. PCS3-D2
Streptomyces sp. PHES57
Streptomyces sp. PKU-EA00015
Streptomyces sp. PLAI1-29
Streptomyces sp. PR69
Streptomyces sp. PRh5
Streptomyces sp. PSAA01
Streptomyces sp. PSKA30
Streptomyces sp. PT12
Streptomyces sp. PTY087I2
Streptomyces sp. PanSC19
Streptomyces sp. PanSC9
Streptomyces sp. PvR006
Streptomyces sp. QHH-9511
Streptomyces sp. QL37
Streptomyces sp. R-46320
Streptomyces sp. R1
Streptomyces sp. R527F
Streptomyces sp. RB17
Streptomyces sp. RCU064
Streptomyces sp. REN17
Streptomyces sp. RG38
Streptomyces sp. RK62
Streptomyces sp. RK76
Streptomyces sp. RKAG290
Streptomyces sp. RKAG293
Streptomyces sp. RKAG337
Streptomyces sp. RLA2-12
Streptomyces sp. RLB1-33
Streptomyces sp. RM99
Streptomyces sp. RO-S4
Streptomyces sp. RP5T
Streptomyces sp. RPA4-2
Streptomyces sp. RPA4-5
Streptomyces sp. RPT161
Streptomyces sp. RS2
Streptomyces sp. RT42
Streptomyces sp. RTd22
Streptomyces sp. Rer75
Streptomyces sp. RerS4
Streptomyces sp. Root1310
Streptomyces sp. Root264
Streptomyces sp. Root369
Streptomyces sp. Ru62
Streptomyces sp. Ru71
Streptomyces sp. Ru72
Streptomyces sp. Ru73
Streptomyces sp. S063
Streptomyces sp. S07_1.15
Streptomyces sp. S10(2018)
Streptomyces sp. S12
Streptomyces sp. SID4-11
Streptomyces sp. S3(2020)
Streptomyces sp. S4.7
Streptomyces sp. S465
Streptomyces sp. S5
Streptomyces sp. S816
Streptomyces sp. S8
Streptomyces sp. SA15
Streptomyces sp. SA3_actF
Streptomyces sp. SA3_actG
Streptomyces sp. SAJ15
Streptomyces sp. SBST2-5
Streptomyces sp. SCA2-2
Streptomyces sp. SDr-06
Streptomyces sp. SHP 1-2
Streptomyces sp. SID10115
Streptomyces sp. SID10362
Streptomyces sp. SID1046
Streptomyces sp. SID10692
Streptomyces sp. SID10853
Streptomyces sp. SID11385
Streptomyces sp. SID12488
Streptomyces sp. SID12501
Streptomyces sp. SID1328
Streptomyces sp. SID13726
Streptomyces sp. SID14436
Streptomyces sp. SID14446
Streptomyces sp. SID14478
Streptomyces sp. SID14515
Streptomyces sp. SID161
Streptomyces sp. SID2119
Streptomyces sp. SID2888
Streptomyces sp. SID2999
Streptomyces sp. SID337
Streptomyces sp. SID486
Streptomyces sp. SID4936
Streptomyces sp. SID4948
Streptomyces sp. SID4950
Streptomyces sp. SID4951
Streptomyces sp. SID4982
Streptomyces sp. SID4985
Streptomyces sp. SID5464
Streptomyces sp. SID5469
Streptomyces sp. SID5470
Streptomyces sp. SID5471
Streptomyces sp. SID5477
Streptomyces sp. SID5606
Streptomyces sp. SID5614
Streptomyces sp. SID5643
Streptomyces sp. SID5785
Streptomyces sp. SID5789
Streptomyces sp. SID5910
Streptomyces sp. SID5914
Streptomyces sp. SID6137
Streptomyces sp. SID6139
Streptomyces sp. SID685
Streptomyces sp. SID69
Streptomyces sp. SID724
Streptomyces sp. SID7958
Streptomyces sp. SID8014
Streptomyces sp. SID8111
Streptomyces sp. SID8352
Streptomyces sp. SID8374
Streptomyces sp. SID8376
Streptomyces sp. SID8380
Streptomyces sp. SID8499
Streptomyces sp. SID9124
Streptomyces sp. SID9913
Streptomyces sp. SKN60
Streptomyces sp. SL13
Streptomyces sp. SL294
Streptomyces sp. SL54
Streptomyces sp. SLBN-115
Streptomyces sp. SLBN-118
Streptomyces sp. SLBN-134
Streptomyces sp. SLBN-31
Streptomyces sp. SM10
Streptomyces sp. SM11
Streptomyces sp. SM13
Streptomyces sp. SM17
Streptomyces sp. SM1
Streptomyces sp. SM9
Streptomyces sp. SMC 277
Streptomyces sp. SP2-10
Streptomyces sp. SPB074
Streptomyces sp. SPB78
Streptomyces sp. SS06011
Streptomyces sp. SS07
Streptomyces sp. SS1-1
Streptomyces sp. SS
Streptomyces sp. ST1020
Streptomyces sp. STCH 565 A
Streptomyces sp. STR69
Streptomyces sp. SUK 48
Streptomyces sp. San01
Streptomyces sp. ScaeMP-6W
Streptomyces sp. ScaeMP-e48
Streptomyces sp. SceaMP-e96
Streptomyces sp. Sge12
Streptomyces sp. SolWspMP-5a-2
Streptomyces sp. SolWspMP-sol7th
Streptomyces sp. T12
Streptomyces sp. TBY4
Streptomyces sp. TLI_105
Streptomyces sp. TLI_146
Streptomyces sp. TLI_171
Streptomyces sp. TLI_185
Streptomyces sp. TLI_235
Streptomyces sp. TLI_55
Streptomyces sp. TM32
Streptomyces sp. TML10
Streptomyces sp. TN58
Streptomyces sp. TOR3209
Streptomyces sp. TP-A0356
Streptomyces sp. TP-A0875
Streptomyces sp. TR1341
Streptomyces sp. TRM64462
Streptomyces sp. TRM68367
Streptomyces sp. TRM68416
Streptomyces sp. TRM75563
Streptomyces sp. TS71-3
Streptomyces sp. TSRI0107
Streptomyces sp. TSRI0261
Streptomyces sp. TSRI0281
Streptomyces sp. TSRI0395
Streptomyces sp. TSRI0445
Streptomyces sp. Tu 2975
Streptomyces sp. Tu 3180
Streptomyces sp. Tu 4128
Streptomyces sp. Tu102
Streptomyces sp. Tu6071
Streptomyces sp. Tue 6075
Streptomyces sp. Tue6028
Streptomyces sp. TverLS-915
Streptomyces sp. UMAF16
Streptomyces sp. UNC401CLCol
Streptomyces sp. UNOB3_S3
Streptomyces sp. V17-9
Streptomyces sp. V2
Streptomyces sp. VITNK9
Streptomyces sp. VMFN-G11Ma
Streptomyces sp. VN1
Streptomyces sp. VRA16 Mangrove soil
Streptomyces sp. W1SF4
Streptomyces sp. WAC 00631
Streptomyces sp. WAC 01325
Streptomyces sp. WAC 01529
Streptomyces sp. WAC 04229
Streptomyces sp. WAC 05379
Streptomyces sp. WAC 06725
Streptomyces sp. WAC 06738
Streptomyces sp. WAC 06783
Streptomyces sp. WAC00263
Streptomyces sp. WAC00303
Streptomyces sp. WAC01280
Streptomyces sp. WAC04114
Streptomyces sp. WAC04189
Streptomyces sp. WAC04770
Streptomyces sp. WAC05292
Streptomyces sp. WAC05374
Streptomyces sp. WAC05950
Streptomyces sp. WAC06273
Streptomyces sp. WAC06614
Streptomyces sp. WAC07061
Streptomyces sp. WAC07149
Streptomyces sp. WAC08452
Streptomyces sp. WELS2
Streptomyces sp. WM6368
Streptomyces sp. WM6372
Streptomyces sp. WM6378
Streptomyces sp. WM6386
Streptomyces sp. WMMB 322
Streptomyces sp. WMMB 714
Streptomyces sp. WMMC940
Streptomyces sp. WZ.A104
Streptomyces sp. Wb2n-11
Streptomyces sp. XY006
Streptomyces sp. XY332
Streptomyces sp. XY511
Streptomyces sp. XY533
Streptomyces sp. XY593
Streptomyces sp. Y2F8-2
Streptomyces sp. YIM 121038
Streptomyces sp. YIM 130001
Streptomyces sp. YIM 132580
Streptomyces sp. YPW6
Streptomyces sp. ZEA17I
Streptomyces sp. ZL-24
Streptomyces sp. alain-838
Streptomyces sp. b84
Streptomyces sp. b94
Streptomyces sp. cf386
Streptomyces sp. e14
Streptomyces sp. endophyte_N2
Streptomyces sp. f150
Streptomyces sp. f51
Streptomyces sp. fd1-xmd
Streptomyces sp. gb14
Streptomyces sp. me109
Streptomyces sp. ms184
Streptomyces sp. ms191
Streptomyces sp. or20
Streptomyces sp. or43
Streptomyces sp. rh34
Streptomyces sp. sk2.1
Streptomyces sp. sk226
Streptomyces sp. st115
Streptomyces sp. st140
Streptomyces sp. st77
Streptomyces sp. t39
Streptomyces sp. t99
Streptomyces sp. uw30
Streptomyces sp. wa1063
Streptomyces sp. yr375
Streptomyces sparsogenes
Streptomyces spectabilis
Streptomyces spinosus
Streptomyces spinoverrucosus
Streptomyces spongiae
Streptomyces spongiicola
Streptomyces sporangiiformans
Streptomyces spororaveus
Streptomyces subrutilus
Streptomyces sudanensis
Streptomyces sviceus
Streptomyces swartbergensis
Streptomyces syringium
Streptomyces tailanensis
Streptomyces taklimakanensis
Streptomyces tanashiensis
Streptomyces tauricus
Streptomyces tendae
Streptomyces thermovulgaris
Streptomyces tirandamycinicus
Streptomyces torulosus
Streptomyces toyocaensis
Streptomyces tricolor
Streptomyces triculaminicus
Streptomyces triticagri
Streptomyces triticiradicis
Streptomyces triticirhizae
Streptomyces triticisoli
Streptomyces tsukubensis
Streptomyces tubercidicus
Streptomyces typhae
Streptomyces umbrinus
Streptomyces uncialis
Streptomyces ureilyticus
Streptomyces varsoviensis
Streptomyces venezuelae
Streptomyces verrucosisporus
Streptomyces vietnamensis
Streptomyces vinaceus
Streptomyces vinaceusdrappus
Streptomyces violace ochromogenes
Streptomyces violaceoruber
Streptomyces violaceorubidus
Streptomyces violaceusniger Tu 4113
Streptomyces violaceusniger group
Streptomyces violaceusniger
Streptomyces violarus
Streptomyces violascens
Streptomyces violens
Streptomyces virginiae
Streptomyces viridiviolaceus
Streptomyces viridochromogenes DSM 40736
Streptomyces viridochromogenes Tue 57
Streptomyces viridochromogenes
Streptomyces viridosporus
Streptomyces wadayamensis
Streptomyces werraensis
Streptomyces wuyuanensis
Streptomyces xanthii
Streptomyces xantholiticus
Streptomyces xanthophaeus
Streptomyces xiaopingdaonensis
Streptomyces xinghaiensis
Streptomyces xylophagus
Streptomyces yangpuensis
Streptomyces yatensis
Streptomyces yerevanensis
Streptomyces yokosukanensis
Streptomyces yunnanensis
Streptomyces zagrosensis
Streptomyces zaomyceticus
Streptomyces zhihengii
Streptomyces zinciresistens
Streptomyces
Streptomycetaceae bacterium MP113-05
Streptosporangiales
Streptosporangium canum
Streptosporangium carneum
Streptosporangium minutum
Streptosporangium roseum DSM 43021
Streptosporangium roseum
Streptosporangium sp. ‘caverna’
Streptosporangium subroseum
Streptosporangium violaceochromogenes
Stutzerimonas azotifigens
Stutzerimonas balearica
Stutzerimonas chloritidismutans AW-1
Stutzerimonas chloritidismutans
Stutzerimonas kirkiae
Stutzerimonas stutzeri A1501
Stutzerimonas stutzeri A15
Stutzerimonas stutzeri B1SMN1
Stutzerimonas stutzeri DSM 10701
Stutzerimonas stutzeri DSM 4166
Stutzerimonas stutzeri KOS6
Stutzerimonas stutzeri MF28
Stutzerimonas stutzeri NF13
Stutzerimonas stutzeri TS44
Stutzerimonas stutzeri
Stutzerimonas xanthomarina
Stutzerimonas
Stylophora pistillata
Subdoligranulum sp. AF14-43
Subdoligranulum sp. AM16-9
Subdoligranulum sp. TF05-17AC
Subdoligranulum variabile
Subtercola boreus
Succinatimonas sp. CAG: 777
Succiniclasticum ruminis DSM 9236
Succiniclasticum ruminis
Succinimonas amylolytica
Succinispira mobilis
Succinivibrio dextrinosolvens DSM 3072
Succinivibrio dextrinosolvens
Sulfitobacter aestuariivivens
Sulfitobacter delicatus
Sulfitobacter guttiformis KCTC 32187
Sulfitobacter guttiformis
Sulfitobacter mediterraneus
Sulfitobacter sp. BSw21498
Sulfitobacter sp. DSM 110093
Sulfitobacter sp. EE-36
Sulfitobacter sp. JL08
Sulfitobacter sp. W027
Sulfobacillus acidophilus DSM 10332
Sulfobacillus acidophilus TPY
Sulfobacillus benefaciens
Sulfobacillus sp. DSM 109850
Sulfobacillus sp. hq2
Sulfobacillus thermosulfidooxidans DSM 9293
Sulfobacillus thermosulfidooxidans
Sulfolobales archaeon SCGC AB-777_J03
Sulfolobus acidocaldarius
Sulfuricella sp. T08
Sulfuricurvum kujiense DSM 16994
Sulfuricurvum kujiense
Sulfuricurvum sp. 17-40-25
Sulfuricurvum sp. 24-42-5
Sulfuricurvum sp. GWF2_44_89
Sulfuricurvum sp. IAE1
Sulfuricurvum sp. MLSB
Sulfuricurvum sp. PC08-66
Sulfuricurvum sp. PD MW2
Sulfuricurvum sp. RIFOXYD12_FULL_44_77
Sulfuricurvum sp. RIFOXYD2_FULL_44_160
Sulfuriferula multivorans
Sulfuriferula nivalis
Sulfuriferula sp. AH1
Sulfurihydrogenibium azorense Az-Ful
Sulfurihydrogenibium azorense
Sulfurihydrogenibium sp.
Sulfurihydrogenibium subterraneum
Sulfurihydrogenibium yellowstonense SS-5
Sulfurihydrogenibium yellowstonense
Sulfurimicrobium lacus
Sulfurimonas gotlandica GD1
Sulfurimonas lithotrophica
Sulfurimonas marina
Sulfurimonas sp. CG01_land_8_20_14_3_00_36_23
Sulfurimonas sp. CG02_land_8_20_14_3_00_36_67
Sulfurimonas sp. CG03_land_8_20_14_0_80_36_25
Sulfurimonas sp. CG07_land_8_20_14_0_80_36_56
Sulfurimonas sp. CG08_land_8_20_14_0_20_36_33
Sulfurimonas sp. CG10_big_fil_rev_8_21_14_0_10_36_24
Sulfurimonas sp. CG12_big_fil_rev_8_21_14_0_65_36_1453
Sulfurimonas sp. CG15_BIG_FIL_POST_REV_8_21_14_020_36_339
Sulfurimonas sp. CG23_combo_of_CG06-09_8_20_14_all_36_33
Sulfurimonas sp. CG_4_10_14_0_2_um_filter_36_1_607
Sulfurimonas sp. CG_4_10_14_3_um_filter_36_910
Sulfurimonas sp. CG_4_8_14_3_um_filter_36_16
Sulfurimonas sp. CG_4_9_14_0_2_um_filter_36_40_7
Sulfurimonas sp. CG_4_9_14_0_8_um_filter_36_38_4
Sulfurimonas sp. HSL-3221
Sulfurimonas sp. UBA12504
Sulfurimonas sp.
Sulfurisoma sediminicola
Sulfuritalea hydrogenivorans
Sulfurivermis fontis
Sulfurospirillum arcachonense
Sulfurospirillum arsenophilum
Sulfurospirillum barnesii SES-3
Sulfurospirillum barnesii
Sulfurospirillum cavolei
Sulfurospirillum deleyianum DSM 6946
Sulfurospirillum deleyianum
Sulfurospirillum diekertiae
Sulfurospirillum halorespirans DSM 13726
Sulfurospirillum multivorans DSM 12446
Sulfurospirillum multivorans
Sulfurospirillum sp. MES
Sulfurospirillum sp. SCADC
Sulfurospirillum sp. SG202
Sulfurospirillum sp. T05
Sulfurospirillum sp. UBA11407
Sulfurospirillum sp. UBA12182
Sulfurospirillum sp. UCH001
Sulfurospirillum
Sulfurovum sp. 35-42-20
Sulfurovum sp. FS08-3
Sulfurovum sp. HSL1-3
Sulfurovum sp. enrichment culture clone C5
Sulfurovum sp.
Sunxiuginia dokdonensis
Sunxiuginia elliptica
Sunxiuginia indica
Sutterella massiliensis
Sutterella sp. 63_29
Sutterella sp. KLE1602
Sutterella wadsworthensis 2_1_59BFAA
Sutterella wadsworthensis
Symbiobacterium thermophilum IAM 14863
Symbiobacterium thermophilum
Symbiodinium microadriaticum
Symbiopectobacterium purcellii
Symploca atlantica PCC 8002
Symplocastrum sp. BBK-W-15
Synechococcus elongatus
Synechococcus nidulans LEGE 07171
Synechococcus sp. 60AY4M2
Synechococcus sp. 63AY4M1
Synechococcus sp. 63AY4M2
Synechococcus sp. 65AY6A5
Synechococcus sp. 65AY6Li
Synechococcus sp. 7002
Synechococcus sp. 8F6
Synechococcus sp. ATX 2A4
Synechococcus sp. BL107
Synechococcus sp. C9
Synechococcus sp. EJ6-Ellesmere
Synechococcus sp. GreenBA-s
Synechococcus sp. JA-2-3B′a(2-13)
Synechococcus sp. JA-3-3Ab
Synechococcus sp. Nb3U1
Synechococcus sp. PCC 11901
Synechococcus sp. PCC 6312
Synechococcus sp. PCC 7003
Synechococcus sp. PCC 7117
Synechococcus sp. PCC 73109
Synechococcus sp. PCC 7335
Synechococcus sp. PCC 7336
Synechococcus sp. PCC 7502
Synechococcus sp. PCC 8807
Synechococcus sp. RS9916
Synechococcus sp. RS9917
Synechococcus sp. RedBA-s
Synechococcus sp. WH 5701
Synechococcus sp. WH 7805
Synechococcus sp. WH 8016
Synechococcus sp.
Synechocystis salina
Synechocystis sp. CACIAM 05
Synechocystis sp. CS-94
Synechocystis sp. FACHB-383
Synechocystis sp. LEGE 06083
Synechocystis sp. PCC 6714
Synechocystis sp. PCC 7338
Synechocystis sp. PCC 7339
Synechocystis sp. PCC 7509
Synechocystis sp. WH 001
Synechocystis sp. WH 002
Synechocystis sp. WH 003
Synergistales bacterium 53_16
Synergistales bacterium 54_24
Synergistales bacterium 54_9
Synergistales bacterium 57_84
Synergistales bacterium 58_81
Synergistes jonesii
Synergistes sp. 3_1_syn1
Synergistetes bacterium HGW-Synergistetes-2
Syntrophaceticus schinkii
Syntrophobacter fumaroxidans MPOB
Syntrophobacter fumaroxidans
Syntrophobacter sp. DG_60
Syntrophobacter sp. SbD1
Syntrophobacteraceae bacterium CG07_land_8_20_14_0_80_61_8
Syntrophobacterales bacterium CG23_combo_of_CG06-09_8_20_14_all_48_27
Syntrophobotulus glycolicus DSM 8271
Syntrophobotulus glycolicus
Syntrophomonas wolfei subsp. wolfei str. Goettingen G311
Syntrophomonas wolfei
Syntrophomonas zehnderi OL-4
Syntrophomonas zehnderi
Syntrophorhabdaceae bacterium PtaU1.Bin034
Syntrophorhabdus aromaticivorans
Syntrophorhabdus sp. PtaU1.Bin050
Syntrophotalea acetylenica
Syntrophotalea acetylenivorans
Syntrophotalea carbinolica DSM 2380
Syntrophotalea carbinolica
Syntrophothermus lipocalidus DSM 12680
Syntrophothermus lipocalidus
Syntrophus aciditrophicus SB
Syntrophus aciditrophicus
Syntrophus gentianae
Tabrizicola algicola
Tabrizicola alkalilacus
Tabrizicola aquatica
Tabrizicola formosa
Tabrizicola oligotrophica
Tabrizicola piscis
Tabrizicola sediminis
Tabrizicola sp. SY72
Tabrizicola sp. TH137
Tabrizicola sp. WMC-M-20
Tahibacter aquaticus
Tahibacter caeni
Taibaiella koreensis
Taibaiella lutea
Taibaiella soli
Talaromyces stipitatus ATCC 10500
Tamlana sp. I1
Tannerella forsythia
Tannockella kyphosi
Tardiphaga robiniae
Tardiphaga sp. 37S4
Tardiphaga sp. OK245
Tardiphaga sp. OK246
Tardiphaga sp. vice 154
Tardiphaga sp. vice 278
Tardiphaga sp. vice 304
Tardiphaga sp. vice 352
Tarenaya hassleriana
Tateyamaria omphalii
Tateyamaria sp. Alg231-49
Telluria mixta
Telluribacter humicola
Telmatospirillum siberiense
Telmatospirillum sp. J64-1
Tenacibaculum adriaticum
Tenacibaculum finnmarkense
Tenacibaculum sp. MAR_2009_124
Tenggerimyces flavus
Tepidanaerobacter acetatoxydans Re1
Tepidanaerobacter acetatoxydans
Tepidanaerobacter syntrophicus
Tepidibacillus decaturensis
Tepidibacillus sp. HK-1
Tepidicella xavieri
Tepidiforma bonchosmolovskayae
Tepidimicrobium xylanilyticum
Tepidiphilus baoligensis
Tepidiphilus margaritifer
Terasakiella brassicae
Terasakiella pusilla
Terasakiella sp. SH-1
Teredinibacter haidensis
Teredinibacter turnerae T7901
Teredinibacter turnerae
Terracoccus luteus
Terribacillus aidingensis
Terribacillus goriensis
Terribacillus halophilus
Terriglobales bacterium
Terriglobus roseus
Terriglobus saanensis
Terrimicrobium sacchariphilum
Terrimonas ferruginea
Terrisporobacter glycolicus
Terrisporobacter othiniensis
Tersicoccus phoenicis
Tessaracoccus aquimaris
Tessaracoccus flavescens
Tetrabaena socialis
Tetrasphaera japonica
Tetzosporium hominis
Thalassobacillus cyri
Thalassobacter sp. 16PALIMAR09
Thalassobacter stenotrophicus DSM 16310
Thalassobacter stenotrophicus
Thalassobacter
Thalassobaculum litoreum DSM 18839
Thalassobaculum litoreum
Thalassobaculum salexigens
Thalassobaculum
Thalassolituus sp. CG17_big_fil_post_rev_8_21_14_2_50_53_8
Thalassomonas actiniarum
Thalassomonas haliotis
Thalassomonas viridans
Thalassoroseus pseudoceratinae
Thalassospira alkalitolerans
Thalassospira australica
Thalassospira lohafexi
Thalassospira lucentensis
Thalassospira mesophila
Thalassospira permensis
Thalassospira povalilytica
Thalassospira sp. A3_1
Thalassospira sp. GB04J01
Thalassospira sp. GO-4
Thalassospira sp. HJ
Thalassospira sp. MCCC 1A02491
Thalassospira xiamenensis
Thalassospira xianhensis
Thalassospira
Thalassotalea algicola
Thalassotalea mangrovi
Thalassotalea sp. PS06
Thauera aminoaromatica
Thauera butanivorans
Thauera linaloolentis
Thauera propionica
Thauera sp. 27
Thauera sp. CAU 1555
Thauera sp. SWB20
Thauera sp. Sel9
Thaumarchaeota archaeon
Thaumasiovibrio occultus
Thaumasiovibrio subtropicus
Theionarchaea archaeon DG-70-1
Theionarchaea archaeon DG-70
Thermacetogenium phaeum DSM 12270
Thermacetogenium phaeum
Thermaerobacter sp. PB12/4term
Thermaerobacter subterraneus DSM 13965
Thermaerobacter subterraneus
Thermanaerosceptrum fracticalcis
Thermanaerovibrio acidaminovorans DSM 6589
Thermanaerovibrio acidaminovorans
Thermasporomyces composti
Thermaurantimonas aggregans
Thermicanus aegyptius
Thermincola ferriacetica
Thermincola potens JR
Thermincola
Thermithiobacillus tepidarius
Thermoanaerobacter brockii subsp. finnii Ako-1
Thermoanaerobacter ethanolicus JW 200
Thermoanaerobacter italicus Ab9
Thermoanaerobacter kivui
Thermoanaerobacter mathranii subsp. mathranii str. A3
Thermoanaerobacter pseudethanolicus ATCC 33223
Thermoanaerobacter siderophilus SR4
Thermoanaerobacter sp. A7A
Thermoanaerobacter sp. X514
Thermoanaerobacter sp. YS13
Thermoanaerobacter thermohydrosulfuricus WC1
Thermoanaerobacter
Thermoanaerobacterales bacterium 50_218
Thermoanaerobacterium aotearoense SCUT27
Thermoanaerobacterium butyriciformans
Thermoanaerobacterium saccharolyticum JW/SL-YS485
Thermoanaerobacterium saccharolyticum
Thermoanaerobacterium sp. PSU-2
Thermoanaerobacterium sp. RBIITD
Thermoanaerobacterium thermosaccharolyticum DSM 571
Thermoanaerobacterium thermosaccharolyticum M0795
Thermoanaerobacterium thermosaccharolyticum
Thermoanaerobacterium xylanolyticum
Thermoanaerobacterium
Thermobacillus composti KWC4
Thermobacillus composti
Thermobaculum terrenum
Thermobifida cellulosilytica
Thermobifida fusca
Thermobifida halotolerans
Thermobispora bispora
Thermobrachium celere DSM 8682
Thermobrachium celere
Thermochromatium tepidum ATCC 43061
Thermochromatium tepidum
Thermoclostridium caenicola
Thermoclostridium stercorarium subsp. leptospartum DSM 9219
Thermoclostridium stercorarium subsp. stercorarium DSM 8532
Thermoclostridium stercorarium subsp. thermolacticum DSM 2910
Thermoclostridium stercorarium
Thermococcales archaeon 44_46
Thermococci archaeon
Thermococcus gammatolerans EJ3
Thermococcus gorgonarius
Thermococcus litoralis DSM 5473
Thermococcus litoralis
Thermocrinis albus DSM 14484
Thermocrinis albus
Thermocrinis ruber
Thermocrispum agreste
Thermocrispum municipale
Thermodesulfatator atlanticus
Thermodesulfatator autotrophicus
Thermodesulfatator indicus DSM 15286
Thermodesulfatator indicus
Thermodesulfitimonas autotrophica
Thermodesulfobacteria bacterium
Thermodesulfobacterium commune DSM 2178
Thermodesulfobacterium commune
Thermodesulfobacterium geofontis OPF15
Thermodesulfobacterium geofontis
Thermodesulfobacterium sp. 37_54
Thermodesulfobacterium thermophilum
Thermodesulfobium acidiphilum
Thermodesulfobium narugense DSM 14796
Thermodesulfobium narugense
Thermodesulfomicrobium sp. WS
Thermodesulforhabdus norvegica
Thermodesulfovibrio aggregans
Thermodesulfovibrio islandicus
Thermodesulfovibrio thiophilus
Thermodesulfovibrio yellowstonii DSM 11347
Thermodesulfovibrio
Thermoflexia bacterium
Thermoflexibacter ruber
Thermoflexus hugenholtzii
Thermogemmata fonticola
Thermogemmatispora aurantia
Thermogemmatispora tikiterensis
Thermogutta terrifontis
Thermohalobacter berrensis
Thermohydrogenium kirishiense
Thermoleptolyngbya sichuanensis PKUAC-SCTA183
Thermoleptolyngbya sichuanensis
Thermoleptolyngbya sp. PKUAC-SCTB121
Thermomicrobium roseum
Thermoleptolyngbya
Thermomonas fusca
Thermomonospora echinospora
Thermomonospora sp. CIF 1
Thermomonospora umbrina
Thermonema lapsum
Thermonema rossianum
Thermoplasmata archaeon HGW-Thermoplasmata-1
Thermoplasmata archaeon M8B2D
Thermoplasmata archaeon M9B1D
Thermoplasmata archaeon M9B2D
Thermoplasmata archaeon
Thermoplasmatales archaeon BRNA1
Thermoplasmatales archaeon I-plasma
Thermoplasmatales archaeon SCGC AB-540-F20
Thermoplasmatales archaeon SG8-52-1
Thermoplasmatales archaeon SG8-52-3
Thermoplasmatales archaeon SG8-52-4
Thermoplasmatales archaeon SM1-50
Thermoplasmatales archaeon ex4484_6
Thermoprotei archaeon
Thermoproteus sp. CIS_19
Thermoproteus sp. CP80
Thermorudis peleae
Thermosediminibacter oceani DSM 16646
Thermosediminibacter oceani
Thermosinus carboxydivorans Nor1
Thermosinus carboxydivorans
Thermosipho melanesiensis
Thermospira aquatica
Thermosporothrix hazakensis
Thermostichus lividus
Thermostichus vulcanus NIES-2134
Thermostichus vulcanus
Thermosulfidibacter takaii ABI70S6
Thermosulfurimonas dismutans
Thermosulfurimonas sp. F29
Thermosulfurimonas
Thermosulfuriphilus ammonigenes
Thermosynechococcus sp. CL-1
Thermosynechococcus sp. HN-54
Thermosynechococcus sp. NK55a
Thermosynechococcus sp. TA-1
Thermosynechococcus vestitus
Thermotalea metallivorans
Thermotoga caldifontis
Thermotoga maritima MSB8
Thermotoga maritima
Thermotoga neapolitana DSM 4359
Thermotoga neapolitana LA10
Thermotoga petrophila RKU-10
Thermotoga petrophila RKU-1
Thermotoga petrophila
Thermotoga sp. 2812B
Thermotoga sp. 47_83
Thermotoga sp. 50_1627
Thermotoga sp. 50_64
Thermotoga sp. EMP
Thermotoga sp. KOL6
Thermotoga sp. RQ2
Thermotoga sp. RQ7
Thermotoga
Thermotogales bacterium 46_20
Thermotogota bacterium
Thermovenabulum gondwanense
Thermovirga lienii DSM 17291
Thermovirga lienii
Thioalbus denitrificans
Thioalkalispira microaerophila
Thioalkalivibrio halophilus
Thioalkalivibrio sp. AKL10
Thioalkalivibrio sp. AKL12
Thioalkalivibrio sp. AKL19
Thioalkalivibrio sp. AKL6
Thioalkalivibrio sp. AKL7
Thioalkalivibrio sp. AKL8
Thioalkalivibrio sp. ALE28
Thioalkalivibrio sp. ALE9
Thioalkalivibrio sp. ALJ15
Thioalkalivibrio sp. ALJ16
Thioalkalivibrio sp. ALJ1
Thioalkalivibrio sp. ALJ24
Thioalkalivibrio sp. ALJ2
Thioalkalivibrio sp. ALJ3
Thioalkalivibrio sp. ALJT
Thioalkalivibrio sp. ALM2T
Thioalkalivibrio sp. ALMg11
Thioalkalivibrio sp. ALMg13-2
Thioalkalivibrio sp. ALMg3
Thioalkalivibrio sp. ALMg9
Thioalkalivibrio sp. ALR17-21
Thioalkalivibrio sp. ALRh
Thioalkalivibrio sp. ALgr1
Thioalkalivibrio sp. ARh3
Thioalkalivibrio sp. HL-Eb18
Thioalkalivibrio sp. K90mix
Thioalkalivibrio sulfidiphilus HL-EbGr7
Thioalkalivibrio thiocyanoxidans
Thioalkalivibrio versutus
Thioalkalivibrio
Thiobaca trueperi
Thiobacillus denitrificans ATCC 25259
Thiobacillus denitrificans
Thiobacillus sp.
Thiobacillus thioparus
Thiocapsa bogorovii
Thiocapsa imhoffii
Thiocapsa marina 5811
Thiocapsa marina
Thiocapsa rosea
Thiocapsa roseopersicina
Thiocapsa sp. 1R
Thiocapsa sp. KS1
Thiocapsa sp.
Thioclava atlantica
Thioclava pacifica
Thioclava sp. DLFJ4-1
Thioclava
Thiococcus pfennigii
Thiocystis minor
Thiocystis violacea
Thiocystis violascens DSM 198
Thiocystis violascens
Thioflavicoccus mobilis 8321
Thioflavicoccus mobilis
Thioflexithrix psekupsensis
Thiohalocapsa halophila
Thiohalocapsa marina
Thiohalocapsa sp. ML1
Thiohalocapsa sp. PB-PSB1
Thiohalospira halophila
Thiolapillus brandeum
Thiomicrorhabdus aquaedulcis
Thiomicrorhabdus arctica
Thiomicrorhabdus sp. Kp2
Thiomicrospira aerophila AL3
Thiomicrospira aerophila
Thiomicrospira microaerophila
Thiomicrospira sp. WB1
Thiomicrospira sp. XS5
Thiomonas arsenitoxydans
Thiomonas bhubaneswarensis
Thiomonas sp. 13-64-67
Thiomonas sp. 20-64-5
Thiomonas sp. FB-6
Thiomonas sp. FB-Cd
Thioploca ingrica
Thiorhodococcus drewsii AZ1
Thiorhodococcus drewsii
Thiorhodococcus mannitoliphagus
Thiorhodococcus minor
Thiorhodospira sibirica ATCC 700588
Thiorhodospira sibirica
Thiorhodovibrio sp. 970
Thiorhodovibrio winogradskyi
Thiospirillum jenense
Thiospirochaeta perfilievii
Thiothrix caldifontis
Thiothrix litoralis
Thiothrix nivea DSM 5205
Thiothrix nivea
Thiothrix subterranea
Thiothrix unzii
Thiothrix
Thiotrichaceae bacterium IS1
Thiovulum sp. ES
Tindallia californiensis
Tindallia magadiensis
Tissierella pigra
Tissierella praeacuta DSM 18095
Tissierella praeacuta
Tissierella simiarum
Tissierellia bacterium S5-A11
Tistrella mobilis KA081020-065
Tistrella mobilis
Tolumonas auensis DSM 9187
Tolumonas auensis
Tolumonas lignilytica
Tolumonas osonensis
Tolypothrix bouteillei VB521301
Tolypothrix bouteillei
Tolypothrix campylonemoides VB511288
Tolypothrix distorta var. symplocoides UTEX B 424
Tolypothrix nodosa Ind17
Tolypothrix sp. ACSSI 038
Tolypothrix sp. FACHB-123
Tolypothrix sp. JCT-1
Tolypothrix sp. LCRNK_26
Tolypothrix sp. LCRNK_2
Tolypothrix sp. LCRNK_3
Tolypothrix sp. LCRNK_5
Tolypothrix sp. LCRNK_8
Tolypothrix sp. LQ-10
Tolypothrix sp. MN-2B
Tolypothrix sp. NIES-4075
Tolypothrix sp. NQAIF319
Tolypothrix sp. PCC 7601
Tolypothrix sp. PCC 7712
Tolypothrix sp. PCC 7910
Tolypothrix sp. UAM 378
Tolypothrix sp. UAM 379
Tolypothrix sp. UAM 413
Tolypothrix sp. UAM 415
Tolypothrix tenuis CG21
Tolypothrix tenuis Ind16
Tolypothrix tenuis PCC 7101
Tolypothrix tenuis
Tomitella biformata
Tomitella gaofuii
Tractidigestivibacter scatoligenes
Tranquillimonas alkanivorans
Tranquillimonas rosea
Trebonia kvetii
Treponema berlinense
Treponema brennaborense DSM 12168
Treponema brennaborense
Treponema bryantii
Treponema denticola AL-2
Treponema denticola ASLM
Treponema denticola ATCC 33520
Treponema denticola ATCC 33521
Treponema denticola ATCC 35404
Treponema denticola ATCC 35405
Treponema denticola F0402
Treponema denticola H-22
Treponema denticola H1-T
Treponema denticola MYR-T
Treponema denticola OTK
Treponema denticola SP23
Treponema denticola SP32
Treponema denticola SP33
Treponema denticola SP37
Treponema denticola SP44
Treponema denticola US-Trep
Treponema denticola
Treponema endosymbiont of Eucomonympha sp.
Treponema lecithinolyticum ATCC 700332
Treponema maltophilum ATCC 51939
Treponema maltophilum
Treponema parvum
Treponema pectinovorum
Treponema porcinum
Treponema primitia ZAS-1
Treponema primitia ZAS-2
Treponema primitia
Treponema ruminis
Treponema saccharophilum DSM 2985
Treponema saccharophilum
Treponema socranskii subsp. paredis ATCC 35535
Treponema socranskii subsp. socranskii VPI DR56BR1116 = ATCC 35536
Treponema socranskii
Treponema sp. AC3
Treponema sp. C6A8
Treponema sp. GWB1_62_6
Treponema sp. J25
Treponema sp. JC4
Treponema sp. Marseille-Q4132
Treponema sp. OMZ 838
Treponema succinifaciens DSM 2489
Treponema succinifaciens
Treponema vincentii ATCC 35580
Treponema vincentii F0403
Treponema vincentii
Treponema zuelzerae
Treponema
Trichlorobacter lovleyi SZ
Trichlorobacter lovleyi
Trichlorobacter thiogenes
Trichocoleus sp. FACHB-262
Trichocoleus sp. FACHB-40
Trichocoleus sp. FACHB-46
Trichocoleus sp. FACHB-591
Trichocoleus sp. FACHB-69
Trichocoleus sp. FACHB-6
Trichocoleus sp. FACHB-832
Trichocoleus sp. FACHB-90
Trichodesmium erythraeum IMS101
Trichodesmium sp. ECS0305
Trichodesmium thiebautii
Trichormus azollae
Trichormus doliolum CG12
Trichormus doliolum Ind1
Trichormus doliolum Ind2
Trichormus sp. MN-6A
Trichormus sp. MN-6B
Trichormus sp. NMC-1
Trichormus variabilis 0441
Trichormus variabilis ATCC 29413
Trichormus variabilis BAC 0502
Trichormus variabilis BAC 9610
Trichormus variabilis BAC 9611
Trichormus variabilis BAC 9721
Trichormus variabilis BAC 9804
Trichormus variabilis BAC 9805
Trichormus variabilis BAC 9817
Trichormus variabilis BAC 9891
Trichormus variabilis BAC 9894
Trichormus variabilis CG4
Trichormus variabilis NIES-23
Trichormus variabilis NOAIF309
Trichormus variabilis SAG 1403-4b
Trichormus variabilis
Trifolium pratense
Trinickia caryophylli
Trinickia symbiotica
Triticum urartu
Tritonibacter litoralis
Tritonibacter mobilis
Tritonibacter multivorans
Tropicibacter naphthalenivorans
Tropicibacter sp. LMIT003
Tropicimonas isoalkanivorans
Tropicimonas sp. IMCC34043
Trypanosoma cruzi marinkellei
Tsukamurella paurometabola
Tsukamurella spumae
Tsukamurella tyrosinosolvens
Tsuneonella flava
Tsuneonella suprasediminis
Tsuneonella troitsensis
Tuberibacillus calidus
Tumebacillus flagellatus
Turicibacter bilis
Turicibacter sanguinis PC909
Turicibacter sanguinis
Turicibacter sp. HGF1
Turicibacter
Turneriella parva DSM 21527
Turneriella parva
Tychonema bourrellyi
Tychonema sp. BBK16
Tychonema sp. LEGE 06208
Tychonema sp. LEGE 07203
Uliginosibacterium aquaticum
Uliginosibacterium gangwonense
Uliginosibacterium sp. TH139
Uliginosibacterium
Umezawaea beigongshangensis
Umezawaea endophytica
Umezawaea tangerina
Undibacter mobilis
Undibacterium flavidum
Undibacterium hunanense
Undibacterium parvum
Undibacterium pigrum
Undibacterium sp. KW1
Undibacterium sp. TS12
Undibacterium sp. YM2
Undibacterium umbellatum
Undibacterium
Urbifossiella limnaea
Ureibacillus manganicus DSM 26584
Ureibacillus manganicus
Ureibacillus massiliensis 4400831 = CIP 108448 = CCUG 49529
Ureibacillus massiliensis
Ureibacillus sinduriensis BLB-1 = JCM 15800
Ureibacillus sinduriensis
Ureibacillus terrenus
Ureibacillus thermosphaericus
Ureibacillus xyleni
Urinicoccus massiliensis
Urinicoccus timonensis
Usitatibacter rugosus
Ustilago maydis 521
Vaginella massiliensis
Vagococcus carniphilus
Vallicoccus soli
Vallitalea guaymasensis
Vallitalea okinawensis
Vallitalea pronyensis
Vallitalea sp. SH18-1
Variibacter gotjawalensis
Variimorphobacter saccharofermentans
Variovorax beijingensis
Variovorax boronicumulans
Variovorax guangxiensis
Variovorax paradoxus
Variovorax soli
Variovorax sp. 369
Variovorax sp. 770b2
Variovorax sp. BK613
Variovorax sp. CF313
Variovorax sp. CY25R-8
Variovorax sp. E3
Variovorax sp. HW608
Variovorax sp. IB41
Variovorax sp. KK3
Variovorax sp. OK605
Variovorax sp. OV329
Variovorax sp. PAMC26660
Variovorax sp. PAMC28562
Variovorax sp. PBL-H6
Variovorax sp. PBS-H4
Variovorax sp. PDC80
Variovorax sp. RA8
Variovorax sp. RO1
Variovorax sp. UMC13
Variovorax sp. VRV01
Variovorax sp. YR750
Variovorax
Varunaivibrio sulfuroxidans
Veillonella parvula
Veillonella ratti
Veillonella seminalis ACS-216-V-Col6b
Veillonella seminalis
Veillonella sp. AF13-2
Veillonella sp. CAG: 933
Veillonella sp.
Veillonella
Veillonellaceae bacterium DNF00626
Veillonellaceae bacterium DNF00751
Verminephrobacter aporrectodeae
Verminephrobacter eiseniae EF01-2
Verminephrobacter eiseniae
Veronia nyctiphanis
Verrucomicrobia bacterium 61-8
Verrucomicrobia bacterium ADurb.Bin122
Verrucomicrobia bacterium S94
Verrucomicrobia bacterium Tous-C9LFEB
Verrucomicrobiae bacterium DG1235
Verrucomicrobiota bacterium
Verrucomicrobiota
Verrucomicrobium sp. 3C
Verrucomicrobium sp. BvORR034
Vibrio aerogenes
Vibrio agarivorans
Vibrio albus
Vibrio alginolyticus
Vibrio astriarenae
Vibrio azureus NBRC 104587
Vibrio bivalvicida
Vibrio brasiliensis LMG 20546
Vibrio brasiliensis
Vibrio campbellii CAIM 519 = NBRC 15631 = ATCC 25920
Vibrio campbellii
Vibrio caribbeanicus ATCC BAA-2122
Vibrio caribbeanicus
Vibrio cholerae 12129(1)
Vibrio cholerae 4260B
Vibrio cholerae B33
Vibrio cholerae BJG-01
Vibrio cholerae BX 330286
Vibrio cholerae CIRS101
Vibrio cholerae CP1030(3)
Vibrio cholerae CP1032(5)
Vibrio cholerae CP1033(6)
Vibrio cholerae CP1035(8)
Vibrio cholerae CP1037(10)
Vibrio cholerae CP1038(11)
Vibrio cholerae CP1040(13)
Vibrio cholerae CP1041(14)
Vibrio cholerae CP1042(15)
Vibrio cholerae CP1044(17)
Vibrio cholerae CP1046(19)
Vibrio cholerae CP1047(20)
Vibrio cholerae CP1048(21)
Vibrio cholerae CP1050(23)
Vibrio cholerae CT 5369-93
Vibrio cholerae HC-02A1
Vibrio cholerae HC-06A1
Vibrio cholerae HC-17A1
Vibrio cholerae HC-17A2
Vibrio cholerae HC-19A1
Vibrio cholerae HC-1A2
Vibrio cholerae HC-20A2
Vibrio cholerae HC-21A1
Vibrio cholerae HC-22A1
Vibrio cholerae HC-23A1
Vibrio cholerae HC-28A1
Vibrio cholerae HC-32A1
Vibrio cholerae HC-33A2
Vibrio cholerae HC-37A1
Vibrio cholerae HC-38A1
Vibrio cholerae HC-39A1
Vibrio cholerae HC-40A1
Vibrio cholerae HC-41A1
Vibrio cholerae HC-41B1
Vibrio cholerae HC-42A1
Vibrio cholerae HC-43A1
Vibrio cholerae HC-43B1
Vibrio cholerae HC-44C1
Vibrio cholerae HC-46A1
Vibrio cholerae HC-47A1
Vibrio cholerae HC-48A1
Vibrio cholerae HC-48B2
Vibrio cholerae HC-49A2
Vibrio cholerae HC-50A2
Vibrio cholerae HC-55A1
Vibrio cholerae HC-55B2
Vibrio cholerae HC-56A2
Vibrio cholerae HC-57A2
Vibrio cholerae HC-60A1
Vibrio cholerae HC-61A1
Vibrio cholerae HC-61A2
Vibrio cholerae HC-62A1
Vibrio cholerae HC-62B1
Vibrio cholerae HC-64A1
Vibrio cholerae HC-65A1
Vibrio cholerae HC-67A1
Vibrio cholerae HC-68A1
Vibrio cholerae HC-69A1
Vibrio cholerae HC-70A1
Vibrio cholerae HC-71A1
Vibrio cholerae HC-72A2
Vibrio cholerae HC-77A1
Vibrio cholerae HC-7A1
Vibrio cholerae HC-80A1
Vibrio cholerae HC-81A1
Vibrio cholerae HC-81A2
Vibrio cholerae HCUF01
Vibrio cholerae HE-45
Vibrio cholerae HE39
Vibrio cholerae HFU-02
Vibrio cholerae IEC224
Vibrio cholerae INDRE 91/1
Vibrio cholerae LMA3984-4
Vibrio cholerae M66-2
Vibrio cholerae MAK 757
Vibrio cholerae MJ-1236
Vibrio cholerae MO10
Vibrio cholerae MS6
Vibrio cholerae NCTC 8457
Vibrio cholerae O1 biovar El Tor str. N16961
Vibrio cholerae O1 str. 116059
Vibrio cholerae O1 str. 116063
Vibrio cholerae O1 str. 2010EL-1786
Vibrio cholerae O1 str. 95412
Vibrio cholerae O1 str. AG-7404
Vibrio cholerae O1 str. AG-8040
Vibrio cholerae O1 str. EC-0009
Vibrio cholerae O1 str. EC-0012
Vibrio cholerae O1 str. EC-0027
Vibrio cholerae O1 str. EC-0051
Vibrio cholerae O1 str. EDC-020
Vibrio cholerae O1 str. EDC-022
Vibrio cholerae O1 str. EM-1536
Vibrio cholerae O1 str. EM-1546
Vibrio cholerae O1 str. EM-1626
Vibrio cholerae O1 str. EM-1676A
Vibrio cholerae O1 str. EM-1727
Vibrio cholerae O1 str. Inaba G4222
Vibrio cholerae O1 str. NHCC-004A
Vibrio cholerae O1 str. NHCC-006C
Vibrio cholerae O1 str. NHCC-008D
Vibrio cholerae O1 str. NHCC-010F
Vibrio cholerae O1 str. Nep-21106
Vibrio cholerae O1 str. Nep-21113
Vibrio cholerae O1 str. PCS-023
Vibrio cholerae O395
Vibrio cholerae PS15
Vibrio cholerae RC27
Vibrio cholerae RC9
Vibrio cholerae VL426
Vibrio cholerae
Vibrio cidicii
Vibrio cincinnatiensis DSM 19608
Vibrio cincinnatiensis
Vibrio coralliilyticus ATCC BAA-450
Vibrio coralliilyticus
Vibrio coralliirubri
Vibrio crassostreae
Vibrio cyclitrophicus
Vibrio diabolicus
Vibrio diazotrophicus
Vibrio fluvialis
Vibrio fortis
Vibrio fujianensis
Vibrio galatheae
Vibrio gallaecicus
Vibrio genomosp. F10
Vibrio hangzhouensis
Vibrio harveyi CAIM 1792
Vibrio harveyi
Vibrio hepatarius
Vibrio hippocampi
Vibrio hyugaensis
Vibrio ichthyoenteri ATCC 700023
Vibrio ichthyoenteri
Vibrio jasicida
Vibrio kanaloae
Vibrio lentus
Vibrio mangrovi
Vibrio marisflavi
Vibrio maritimus
Vibrio mediterranei AK1
Vibrio mediterranei
Vibrio metoecus
Vibrio mexicanus
Vibrio mimicus CAIM 602
Vibrio mimicus SX-4
Vibrio mimicus VM573
Vibrio mimicus
Vibrio mytili
Vibrio natriegens NBRC 15636 = ATCC 14048 = DSM 759
Vibrio natriegens
Vibrio nigripulchritudo
Vibrio orientalis CIP 102891 = ATCC 33934
Vibrio owensii
Vibrio pacinii
Vibrio paracholerae HE-09
Vibrio paracholerae HE-16
Vibrio paracholerae
Vibrio parahaemolyticus 10290
Vibrio parahaemolyticus 10296
Vibrio parahaemolyticus 10329
Vibrio parahaemolyticus 12310
Vibrio parahaemolyticus 12315
Vibrio parahaemolyticus 3256
Vibrio parahaemolyticus 3324
Vibrio parahaemolyticus 846
Vibrio parahaemolyticus 949
Vibrio parahaemolyticus 97-10290
Vibrio parahaemolyticus AQ3810
Vibrio parahaemolyticus EN2910
Vibrio parahaemolyticus EN9701173
Vibrio parahaemolyticus EN9901310
Vibrio parahaemolyticus IDH02640
Vibrio parahaemolyticus NIHCB0757
Vibrio parahaemolyticus UCM-V493
Vibrio parahaemolyticus V-223/04
Vibrio parahaemolyticus VP-NY4
Vibrio parahaemolyticus VP232
Vibrio parahaemolyticus VPCR-2009
Vibrio parahaemolyticus VPCR-2010
Vibrio parahaemolyticus
Vibrio plantisponsor
Vibrio ponticus
Vibrio porteresiae DSM 19223
Vibrio porteresiae
Vibrio proteolyticus NBRC 13287
Vibrio quintilis
Vibrio renipiscarius
Vibrio rhizosphaerae
Vibrio salinus
Vibrio scophthalmi LMG 19158
Vibrio scophthalmi
Vibrio sinaloensis DSM 21326
Vibrio sinaloensis
Vibrio sp. 10N.286.49.B3
Vibrio sp. 16
Vibrio sp. AND4
Vibrio sp. C7
Vibrio sp. EJY3
Vibrio sp. F12 FF_152
Vibrio sp. HA2012
Vibrio sp. JCM 18905
Vibrio sp. JCM 19052
Vibrio sp. MEBiC08052
Vibrio sp. MSSRF39
Vibrio sp. N418
Vibrio sp. RC586
Vibrio sp. S4B1
Vibrio sp. STUT-A11
Vibrio sp. VPAP30
Vibrio sp. dhg
Vibrio sp. kj40-1
Vibrio sp. qd031
Vibrio splendidus ATCC 33789
Vibrio splendidus
Vibrio tapetis subsp. tapetis
Vibrio toranzoniae
Vibrio tritonius
Vibrio tubiashii ATCC 19109
Vibrio tubiashii NCIMB 1337 = ATCC 19106
Vibrio tubiashii
Vibrio ulleungensis
Vibrio variabilis
Vibrio viridaestus
Vibrio vulnificus YJ016
Vibrio vulnificus
Vibrio xiamenensis
Vibrio ziniensis
Vibrio
Vigna angularis var. angularis
Vigna angularis
Vineibacter terrae
Virgibacillus dokdonensis
Virgibacillus halodenitrificans
Virgibacillus massiliensis
Virgibacillus salinus
Virgibacillus senegalensis
Virgibacillus soli
Virgibacillus sp. Bac330
Virgibacillus sp. CM-4
Virgibacillus sp. MSP 4-1
Virgibacillus sp. SK37
Virgibacillus subterraneus
Virgibacillus
Virgisporangium aliadipatigenens
Virgisporangium aurantiacum
Viridibacillus arvi
Vitiosangium sp. GDMCC 1.1324
Vitreoscilla filiformis
Vogesella oryzae
Vogesella sp. EB
Vogesella sp. LIG4
Vulcanibacillus modesticaldus
Vulcanococcus limneticus
Vulgatibacter incomptus
Waddlia chondrophila 2032/99
Waddlia chondrophila WSU 86-1044
Waddlia chondrophila
Wansuia hejianensis
Waterburya agarophytonicola
Weeksella
Weissella oryzae
Weizmannia acidiproducens
Weizmannia coagulans P38
Weizmannia coagulans
Weizmannia ginsengihumi
Wenjunlia tyrosinilytica
Wenzhouxiangella sp. XN201
Westiellopsis prolifica
Westiellopsis sp. CG19
Westiellopsis sp. Ind19
Westiellopsis sp. Ind20
Westiellopsis sp. MN-12B
Westiellopsis sp. MN-8A
Westiellopsis sp. NQAIF324
Williamsia deligens
Williamsia limnetica
Williamsia marianensis
Williamsia muralis
Williamsia serinedens
Williamsia soli
Williamsia sp. 1138
Williamsia sp. CHRR-6
Williamsia sp. D3
Williamsia sterculiae
Winogradskyella sp. E313
Winogradskyella sp. J14-2
Wolinella succinogenes
Wollea ambigua LH-M
Wollea ambigua SAG 1403-7
Xanthobacter agilis
Xanthobacter autotrophicus Py2
Xanthobacter autotrophicus
Xanthobacter dioxanivorans
Xanthobacter flavus
Xanthobacter sp. 126
Xanthobacter sp. YC-JY1
Xanthobacter tagetidis
Xanthobacter
Xanthomonas albilineans
Xanthomonas alfalfae
Xanthomonas arboricola pv. celebensis
Xanthomonas arboricola pv. populi
Xanthomonas arboricola pv. pruni MAFF 301427
Xanthomonas arboricola pv. pruni str. MAFF 311562
Xanthomonas arboricola
Xanthomonas axonopodis pv. bauhiniae
Xanthomonas axonopodis pv. cajani
Xanthomonas axonopodis pv. clitoriae
Xanthomonas axonopodis pv. khayae
Xanthomonas axonopodis pv. melhusii
Xanthomonas axonopodis
Xanthomonas bonasiae
Xanthomonas bromi
Xanthomonas campestris pv. centellae
Xanthomonas campestris pv. musacearum NCPPB 4384
Xanthomonas campestris pv. musacearum NCPPB 4392
Xanthomonas campestris pv. raphani 756C
Xanthomonas campestris pv. vitiscarnosae
Xanthomonas campestris pv. vitistrifoliae
Xanthomonas campestris pv. vitiswoodrowii
Xanthomonas campestris
Xanthomonas cannabis pv. cannabis
Xanthomonas cannabis
Xanthomonas cassavae
Xanthomonas citri pv. bilvae
Xanthomonas citri pv. malvacearum str. GSPB2388
Xanthomonas citri pv. mangiferaeindicae LMG 941
Xanthomonas citri pv. viticola
Xanthomonas citri
Xanthomonas codiaei
Xanthomonas cucurbitae
Xanthomonas dyei
Xanthomonas euroxanthea
Xanthomonas euvesicatoria
Xanthomonas floridensis
Xanthomonas fragariae
Xanthomonas hortorum
Xanthomonas hyacinthi DSM 19077
Xanthomonas hydrangeae
Xanthomonas indica
Xanthomonas maliensis
Xanthomonas melonis
Xanthomonas nasturtii
Xanthomonas oryzae pv. oryzae
Xanthomonas oryzae
Xanthomonas perforans
Xanthomonas phaseoli pv. phaseoli
Xanthomonas phaseoli
Xanthomonas pisi
Xanthomonas populi
Xanthomonas prunicola
Xanthomonas sacchari
Xanthomonas sontii
Xanthomonas sp. 3272
Xanthomonas sp. 3376
Xanthomonas sp. 3498
Xanthomonas sp. A1809
Xanthomonas sp. A2111
Xanthomonas sp. AM6
Xanthomonas sp. BRIP62409
Xanthomonas sp. BRIP62411
Xanthomonas sp. BRIP62415
Xanthomonas sp. CFBP 8443
Xanthomonas sp. CFBP 8445
Xanthomonas sp. D-109
Xanthomonas sp. D-93
Xanthomonas sp. GPE 39
Xanthomonas sp. GW
Xanthomonas sp. JAI131
Xanthomonas sp. LMG 12459
Xanthomonas sp. LMG 12460
Xanthomonas sp. LMG 12461
Xanthomonas sp. LMG 12462
Xanthomonas sp. LMG 8992
Xanthomonas sp. LMG 9002
Xanthomonas sp. Leaf131
Xanthomonas sp. MLO165
Xanthomonas sp. MUS 060
Xanthomonas sp. MWU16-30325
Xanthomonas sp. NCPPB 1128
Xanthomonas sp. SHU 166
Xanthomonas sp. SHU 199
Xanthomonas sp. SHU 308
Xanthomonas sp. SI
Xanthomonas sp. SS
Xanthomonas sp. XNM01
Xanthomonas sp.
Xanthomonas surreyensis
Xanthomonas theicola
Xanthomonas translucens
Xanthomonas vasicola pv. vasculorum NCPPB 1326
Xanthomonas vasicola
Xanthomonas vesicatoria
Xanthomonas
Xaviernesmea oryzae
Xenococcus sp. PCC 7305
Xenophilus arseniciresistens
Xenophilus sp. AP218F
Xenophilus sp. E41
Xenophilus sp. L33
Xenophilus sp. Marseille-Q4582
Xenorhabdus hominickii
Xinfangfangia humi
Xinfangfangia pollutisoli
Xylanibacterium ulmi
Xylariales sp. No. 14919
Xylella fastidiosa subsp. multiplex
Xylophilus ampelinus
Xylophilus rhododendri
Xylophilus sp. ASV27
Xylophilus sp. Leaf220
Yangia pacifica
Yeosuana marina
Yersinia pestis
Yersinia pseudotuberculosis
Yimella lutea
Yimella sp. NH-Cas1
Yimella sp. RIT 621
Yimella sp. cx-51
Yinghuangia sp. ASG 101
Yonghaparkia alkaliphila
Yonghaparkia
Yoonia litorea
Yoonia maricola
Yoonia rosea
Yoonia sediminilitoris
Yoonia sp. F2084L
Yoonia vestfoldensis SKA53
Yoonia vestfoldensis
Youngiibacter fragilis 232.1
Youngiibacter fragilis
Zarconia navalis
Zavarzinella formosa
Zavarzinia aquatilis
Zavarzinia marina
Zea mays
Zehria floridana WH 8904
Zehria sp. KO11DG
Zehria sp. KO68DGA
Zehria sp. SK40
Zehria sp. SKTU126
Zetaproteobacteria bacterium CG02_land_8_20_14_3_00_50_9
Zetaproteobacteria bacterium CG03_land_8_20_14_0_80_59_51
Zetaproteobacteria bacterium CG06_land_8_20_14_3_00_59_53
Zetaproteobacteria bacterium CG08_land_8_20_14_0_20_55_17
Zetaproteobacteria bacterium CG11_big_fil_rev_8_21_14_0_20_59_439
Zetaproteobacteria bacterium CG12_big_fil_rev_8_21_14_0_65_54_13
Zetaproteobacteria bacterium CG12_big_fil_rev_8_21_14_0_65_55_1124
Zetaproteobacteria bacterium CG17_big_fil_post_rev_8_21_14_2_50_50_13
Zetaproteobacteria bacterium CG23_combo_of_CG06-09_8_20_14_all_54_7
Zetaproteobacteria bacterium CG23_combo_of_CG06-09_8_20_14_all_59_86
Zetaproteobacteria bacterium CG2_30_59_37
Zetaproteobacteria bacterium CG_4_10_14_0_2_um_filter_55_2_0
Zetaproteobacteria bacterium CG_4_10_14_0_8_um_filter_49_8_0
Zetaproteobacteria bacterium CG_4_10_14_0_8_um_filter_55_4_3
Zetaproteobacteria bacterium CG_4_10_14_0_8_um_filter_59_1_27
Zetaproteobacteria bacterium CG_4_10_14_3_um_filter_54_28
Zetaproteobacteria bacterium CG_4_8_14_3_um_filter_59_5
Zetaproteobacteria bacterium CG_4_9_14_0_2_um_filter_59_19_1
Zetaproteobacteria bacterium CG_4_9_14_0_8_um_filter_55_31
Zetaproteobacteria bacterium CG_4_9_14_3_um_filter_54_145
Zetaproteobacteria bacterium
Zhihengliuella flava
Zhihengliuella salsuginis
Zhongshania aliphaticivorans
Zhouia spongiae
Zobellella denitrificans
Zoogloea dura
Zoogloea oleivorans
Zoogloea oryzae
Zoogloea sp. 1C4
Zoogloea sp. LCSB751
Zoogloea sp.
Zoogloeaceae bacteirum Par-f-2
Zostera marina
Zuelzera stenostrepta
Zunongwangia profunda
Zymomonas mobilis subsp. mobilis ATCC 10988
Zymomonas mobilis subsp. mobilis NCIMB 11163
Zymomonas mobilis subsp. mobilis NRRL B-12526
Zymomonas mobilis subsp. mobilis ZM4 = ATCC 31821
Zymomonas mobilis subsp. mobilis str. CP4 = NRRL B-14023
Zymomonas mobilis subsp. mobilis
Zymomonas mobilis subsp. pomaceae ATCC 29192
Zymomonas mobilis subsp. pomaceae
Zymomonas mobilis
Acidovorax ebreus TPSY
Arcobacter porcinus
Bacillus enclensis
Bacillus selenitireducens MLS10
Bacillus sp. KCTC 13219
Bacteroides pectinophilus ATCC 43243
Butyribacterium methylotrophicum
Clostridium aminophilum
Clostridium bifermentans ATCC 638
Clostridium cellulosi
Clostridium citroniae WAL-19142
Clostridium fimetarium
Clostridium innocuum
Clostridium leptum DSM 753
Clostridium leptum
Clostridium methoxybenzovorans
Clostridium polysaccharolyticum
Clostridium sordellii VPI 9048
Clostridium sordellii
Clostridium sphenoides JCM 1415
Clostridium symbiosum
Clostridium ultunense Esp
Eubacterium cellulosolvens
Eubacterium eligens ATCC 27750
Eubacterium infirmum
Eubacterium rectale DSM 17629
Eubacterium rectale
Eubacterium siraeum
Eubacterium tenue
Eubacterium yurii subsp. margaretiae ATCC 43715
Eubacterium yurii
Flexibacter sp. ATCC 35103
Flexibacter sp. ATCC 35208
Halarcobacter arenosus
Kluyvera intestini
Leptolyngbya sp. JSC-1
Luteovulum sphaeroides subsp. megalophilum
Micrococcus luteus ATCC 49442
Mycobacterium fortunisiensis
Mycobacterium stephanolepidis
Phormidium sp. ETS-05
Pseudomonas nosocomialis
Pseudomonas zhaodongensis
Ruminococcus torques L2-14
Ruminococcus torques
Scytonema hofmanni UTEX 2349
actinomycete 7501
actinomycete L3
aff. Roholtiella sp. LEGE 12411
alpha proteobacterium AAP81b
alpha proteobacterium BAL398
alpha proteobacterium IMCC14465
alpha proteobacterium R45977
anaerobic digester metagenome
archaeon BMS3Abin16
archaeon BMS3Bbin15
archaeon BMS3Bbin16
archaeon GW2011_AR16
archaeon HR01
archaeon HR02
archaeon HR03
archaeon
bacterium 0.1xD8-82
bacterium 1XD42-54
bacterium 1xD42-67
bacterium 42_11
bacterium BFN5
bacterium BMS3Abin07
bacterium BMS3Abin08
bacterium BMS3Abin10
bacterium BMS3Abinll
bacterium BMS3Abin13
bacterium BMS3Bbin05
bacterium BMS3Bbin06
bacterium BMS3Bbin07
bacterium BMS3Bbin08
bacterium BMS3Bbin14
bacterium BRH_c32
bacterium CG_4_9_14_3_um_filter_65_15
bacterium CZ152S
bacterium DZY-HS14
bacterium DZY-N56
bacterium F16
bacterium G25-1-2
bacterium G25-1
bacterium HR13
bacterium HR19
bacterium HR26
bacterium HR28
bacterium HR30
bacterium JGI 053
bacterium M00.F.Ca.ET.141.01.1.1
bacterium M00.F.Ca.ET.146.01.1.1
bacterium M00.F.Ca.ET.152.01.1.1
bacterium M00.F.Ca.ET.156.01.1.1
bacterium M00.F.Ca.ET.157.01.1.1
bacterium M00.F.Ca.ET.159.01.1.1
bacterium M00.F.Ca.ET.162.01.1.1
bacterium M00.F.Ca.ET.163.01.1.1
bacterium M00.F.Ca.ET.177.01.1.1
bacterium M00.F.Ca.ET.199.01.1.1
bacterium M00.F.Ca.ET.205.01.1.1
bacterium M00.F.Ca.ET.221.01.1.1
bacterium M00.F.Ca.ET.222.01.1.1
bacterium M00.F.Ca.ET.227.01.1.1
bacterium M00.F.Ca.ET.230.01.1.1
bacterium Q25-2
bacterium SM23_31
bacterium SM23_57
bacterium TMED88
bacterium Y41
bacterium enrichment culture clone ST1
bacterium enrichment culture clone ST2
bacterium enrichment culture clone ST3
bacterium enrichment culture clone ST4
bacterium enrichment culture clone ST5
bacterium
beta proteobacterium AAP121
beta proteobacterium AAP51
beta proteobacterium AAP65
beta proteobacterium AAP99
candidate division KSB1 bacterium 4484_188
candidate division KSB1 bacterium
candidate division KSB3 bacterium
candidate division MSBL1 archaeon SCGC-AAA259B11
candidate division MSBL1 archaeon SCGC-AAA259D14
candidate division MSBL1 archaeon SCGC-AAA259I07
candidate division MSBL1 archaeon SCGC-AAA261D19
candidate division TA06 bacterium 32_111
candidate division TA06 bacterium 34_109
candidate division TA06 bacterium DG_24
candidate division TA06 bacterium DG_26
candidate division TA06 bacterium SM1_40
candidate division TA06 bacterium SM23_40
candidate division WOR-1 bacterium DG_54_3
candidate division WOR-3 bacterium JGI_Cruoil_03_44_89
candidate division WOR-3 bacterium JGI_Cruoil_03_51_56
candidate division WOR-3 bacterium
candidate division WOR_3 bacterium SM23_42
candidate division WOR_3 bacterium SM23_60
candidate division Zixibacteria bacterium 4484_95
candidate division Zixibacteria bacterium DG_27
candidate division Zixibacteria bacterium HGW-Zixibacteria-1
candidate division Zixibacteria bacterium SM23_73
candidate division Zixibacteria bacterium SM1_73
candidate division Zixibacteria bacterium SM23_81
candidate division Zixibacteria bacterium
cf. Phormidesmis sp. LEGE 11477
cyanobacterium KM9
cyanobacterium TDX16
cyanobacterium endosymbiont of Epithemia sp. CRS-2021a
cyanobacterium endosymbiont of Epithemia sp. CRS-2021b
cyanobacterium endosymbiont of Epithemia turgida isolate EtSB Lake Yunoko
cyanobacterium endosymbiont of Epithemia turgida
cyanobacterium endosymbiont of Rhopalodia gibba
cyanobacterium endosymbiont of Rhopalodia gibberula
delta proteobacterium ML8_F1
delta proteobacterium NaphS2
endosymbiont of Lamellibrachia barhami
endosymbiont of Ridgeia piscesae
endosymbiont of Riftia pachyptila (vent Ph05)
endosymbiont of Tevnia jerichonana (vent Tica)
endosymbiont of unidentified scaly snail isolate Monju
enrichment culture bacterium 01
enrichment culture bacterium 02
enrichment culture bacterium 37
enrichment culture
filamentous cyanobacterium CCP1
filamentous cyanobacterium CCP2
filamentous cyanobacterium CCP4
filamentous cyanobacterium CCT1
filamentous cyanobacterium ESFC-1
filamentous thermophilic cyanobacterium FTC1
filamentous thermophilic cyanobacterium FTC2
filamentous thermophilic cyanobacterium tBTRCCn 101
filamentous thermophilic cyanobacterium tBTRCCn 24
filamentous thermophilic cyanobacterium tBTRCCn 301
gamma proteobacterium BAL281
gamma proteobacterium BAL286
gamma proteobacterium BAL354
gamma proteobacterium BAL361
gamma proteobacterium BAL374
gamma proteobacterium BAL376
gamma proteobacterium BAL386
gamma proteobacterium BAL407
gamma proteobacterium BAL410
gamma proteobacterium BAL501
gamma proteobacterium BAL502
gamma proteobacterium BAL503
gamma proteobacterium BAL504
gamma proteobacterium BAL505
gamma proteobacterium BAL506
gamma proteobacterium BAL507
gamma proteobacterium BAL508
gamma proteobacterium BAL509
gamma proteobacterium BAL510
gamma proteobacterium BAL511
gamma proteobacterium BAL512
gamma proteobacterium BAL513
gamma proteobacterium BAL514
gamma proteobacterium BAL515
gamma proteobacterium BAL516
gamma proteobacterium BDW918
gamma proteobacterium HIMB55
gamma proteobacterium HdN1
gamma proteobacterium IMCC1989
gamma proteobacterium NOR5-3
gamma proteobacterium enrichment culture clone GD11.0
gamma proteobacterium enrichment culture clone GD11.10
gamma proteobacterium enrichment culture clone GD11.11
gamma proteobacterium enrichment culture clone GD11.12
gamma proteobacterium enrichment culture clone GD11.13
gamma proteobacterium enrichment culture clone GD11.14
gamma proteobacterium enrichment culture clone GD11.15
gamma proteobacterium enrichment culture clone GD11.16
gamma proteobacterium enrichment culture clone GD11.17
gamma proteobacterium enrichment culture clone GD11.18
gamma proteobacterium enrichment culture clone GD11.19
gamma proteobacterium enrichment culture clone GD11.1
gamma proteobacterium enrichment culture clone GD11.20
gamma proteobacterium enrichment culture clone GD11.21
gamma proteobacterium enrichment culture clone GD11.22
gamma proteobacterium enrichment culture clone GD11.23
gamma proteobacterium enrichment culture clone GD11.24
gamma proteobacterium enrichment culture clone GD11.2
gamma proteobacterium enrichment culture clone GD11.3
gamma proteobacterium enrichment culture clone GD11.4
gamma proteobacterium enrichment culture clone GD11.5
gamma proteobacterium enrichment culture clone GD11.6
gamma proteobacterium enrichment culture clone GD11.7
gamma proteobacterium enrichment culture clone GD11.8
gamma proteobacterium enrichment culture clone GD11.9
gamma proteobacterium symbiont of Ctena orbiculata
gamma proteobacterium symbiont of Stewartia floridana
groundwater metagenome
heterocystous cyanobacterium LD-B
hot springs metagenome
human gut metagenome
hydrocarbon metagenome
hydrothermal vent metagenome
hyperthermophilic methanogen FS406-22
lactate SRB-Enrichment culture clone HBLac1
mangrove root bacterium HS001
marine gamma proteobacterium HTCC2080
marine gamma proteobacterium HTCC2143
marine gamma proteobacterium HTCC2207
marine metagenome
marine proteobacterium ‘Bird Shoal 1-2’
marine proteobacterium ‘Bird Shoal 1-31’
marine proteobacterium ‘Bird Shoal 1-9’
marine proteobacterium ‘Bird Shoal 12-2’
marine proteobacterium ‘Bird Shoal 4-6’
marine proteobacterium ‘Bird Shoal 6-2’
marine proteobacterium ‘Bird Shoal 8-1’
marine proteobacterium ‘Bird Shoal sa3’
marine proteobacterium ‘Bird Shoal sa4’
marine proteobacterium ‘Bird Shoal sw4’
marine proteobacterium ‘Sippewissett 2’
marine proteobacterium ‘Sippewissett 2-21’
marine proteobacterium ‘Sippewissett 2-32’
marine proteobacterium ‘Sippewissett 24’
marine proteobacterium ‘Tomales Bay 13-32r’
marine proteobacterium ‘Tomales Bay 14-24’
marine proteobacterium ‘Tomales Bay 2-31’
marine proteobacterium ‘Tomales Bay 5-24’
marine proteobacterium ‘Tomales Bay lg wh’
marine proteobacterium ‘Tomales Bay med white’
marine proteobacterium ‘Tomales Bay mg10’
marine proteobacterium ‘Tomales Bay mg15’
marine proteobacterium ‘Tomales Bay wc1-2 sm white’
marine proteobacterium ‘Tomales Bay wc1-28’
marine proteobacterium ‘Tomales Bay wc2-3 lg wh’
marine proteobacterium ‘Tomales Bay wc2-3 sm wh’
marine proteobacterium ‘Tomales Bay wh clear’
marine proteobacterium ‘Tomales Bay wh’
marine proteobacterium L2-3
marine proteobacterium WC1-2(LW)
marine proteobacterium WC1-2(sm)
marine stromatolite cyanobacterium HBC2C2
marine stromatolite cyanobacterium HBC5C3
marine stromatolite cyanobacterium HBC6C3
marine stromatolite eubacterium HB(0697) A06
marine stromatolite eubacterium HB(0697) A100
marine stromatolite eubacterium HB(0697) A101A
marine stromatolite eubacterium HB(0697) C06A
marine stromatolite eubacterium HB(0697) C102
marine stromatolite eubacterium HB(0697) C104
marine stromatolite eubacterium HB(0898) A101
marine stromatolite eubacterium HB(0898) A69
marine stromatolite eubacterium HB(0898) C03
marine stromatolite eubacterium HB(0898) C06
marine stromatolite eubacterium HB(0898) Z02
marine stromatolite eubacterium HB(0898) Z05
marine stromatolite eubacterium HB(0898) Z07
marine stromatolite eubacterium HB(0898) Z12
marine stromatolite eubacterium HB(0898) Z14
methanogenic archae on ISO4-H5
methanogenic archaeon mixed culture ISO4-G1
methanotrophic bacterial endosymbiont of Bathymodiolus sp.
mine drainage metagenome
miscellaneous Crenarchaeota group archaeon SMTZ1-55
miscellaneous Crenarchaeota group-1 archaeon SG8-32-1
miscellaneous Crenarchaeota group-6 archaeon AD8-1
nitrogen fixing bacterium NKNRT11
nitrogen fixing bacterium NKNRT16
nitrogen fixing bacterium NKNRT2-2
nitrogen fixing bacterium NKNRT2-7
nitrogen fixing bacterium NKNRT2-8
nitrogen fixing bacterium NKNRT23
nitrogen fixing bacterium NKNRT26
nitrogen fixing bacterium NKNRT27
nitrogen fixing bacterium NKNRT6
nitrogen-fixing bacterium HS002
nitrogen-fixing bacterium JC110
nitrogen-fixing bacterium KNUC115
nitrogen-fixing bacterium SC16
nitrogen-fixing bacterium SG21
nitrogen-fixing bacterium TS210
sulfur-oxidizing endosymbiont of Gigantopelta aegis
synthetic construct
temperate forest soil bacterium AC06
temperate forest soil bacterium YC06
temperate forest soil bacterium YC07
unclassified Achromobacter
unclassified Acidovorax
unclassified Acinetobacter
unclassified Actinomyces
unclassified Actinopolyspora
unclassified Actinotalea
unclassified Adlercreutzia
unclassified Aeromicrobium
unclassified Agrobacterium
unclassified Alcanivorax
unclassified Alteromonas
unclassified Amycolatopsis
unclassified Anabaena
unclassified Anaeromyxobacter
unclassified Anaerotruncus
unclassified Arthrobacter
unclassified Aureimonas
unclassified Azospirillum
unclassified Blastococcus
unclassified Blastopirellula
unclassified Bradyrhizobium
unclassified Brenneria
unclassified Burkholderia
unclassified Butyrivibrio
unclassified Caballeronia
unclassified Calothrix
unclassified Caulobacter
unclassified Cellulomonas
unclassified Cellvibrio
unclassified Chitinophaga
unclassified Chryseobacterium
unclassified Citromicrobium
unclassified Clostridioides
unclassified Clostridium
unclassified Coleofasciculus
unclassified Colwellia
unclassified Coprococcus
unclassified Corallococcus
unclassified Cryobacterium
unclassified Cupriavidus
unclassified Curtobacterium
unclassified Dehalobacter
unclassified Desertifilum
unclassified Desulfovibrio
unclassified Dietzia
unclassified Dorea
unclassified Duganella
unclassified Ectothiorhodospira
unclassified Exiguobacterium
unclassified Fibrobacter
unclassified Flavobacterium
unclassified Flavonifractor
unclassified Frankia
unclassified Frigoribacterium
unclassified Frondihabitans
unclassified Fusibacter
unclassified Gemmata
unclassified Geobacillus
unclassified Geothrix
unclassified Gordonia (in: high G + C Gram-positive bacteria)
unclassified Haloarcula
unclassified Halomicroarcula
unclassified Halomonas
unclassified Herbaspirillum
unclassified Herbiconiux
unclassified Hyphomicrobium
unclassified Idiomarina
unclassified Jannaschia
unclassified Janthinobacterium
unclassified Klebsiella
unclassified Kribbella
unclassified Laspinema
unclassified Lebetimonas
unclassified Leifsonia
unclassified Leisingera
unclassified Lentimonas
unclassified Leptolyngbya
unclassified Limnothrix
unclassified Loktanella
unclassified Luteimonas
unclassified Marinobacter
unclassified Marinobacterium
unclassified Martelella
unclassified Massilia
unclassified Megasphaera
unclassified Mesorhizobium
unclassified Mesotoga
unclassified Methanosarcina
unclassified Methylobacterium
unclassified Methylocaldum
unclassified Methylocystis
unclassified Methylomonas
unclassified Methylosinus
unclassified Methyloversatilis
unclassified Microbacterium
unclassified Micrococcus
unclassified Microcoleus
unclassified Microvirga
unclassified Modestobacter
unclassified Mycobacterium
unclassified Mycolicibacter
unclassified Mycolicibacterium
unclassified Myxococcus
unclassified Nannocystis
unclassified Neorhizobium
unclassified Nesterenkonia
unclassified Nocardia
unclassified Nocardioides
unclassified Nocardiopsis
unclassified Nodularia (in: cyanobacteria)
unclassified Nostoc
unclassified Paenibacillus
unclassified Pannonibacter
unclassified Pantoea
unclassified Paraburkholderia
unclassified Paracoccus (in: a-proteobacteria)
unclassified Pedobacter
unclassified Pelomonas
unclassified Phenylobacterium
unclassified Photobacterium
unclassified Planktothricoides
unclassified Planktothrix
unclassified Plantibacter
unclassified Pleomorphomonas
unclassified Polaromonas
unclassified Poseidonibacter
unclassified Pseudoalteromonas
unclassified Pseudoclavibacter
unclassified Pseudodesulfovibrio
unclassified Pseudomonas
unclassified Pseudonocardia
unclassified Psychrobacter
unclassified Psychrosphaera
unclassified Pyramidobacter
unclassified Raoultella
unclassified Rhizobium
unclassified Rhodobacter
unclassified Rhodococcus
unclassified Rhodopseudomonas
unclassified Romboutsia
unclassified Rubellimicrobium
unclassified Ruminococcus
unclassified Salinibacterium
unclassified Schaalia
unclassified Scytonema
unclassified Selenomonas
unclassified Simplicispira
unclassified Sphingomonas
unclassified Stenotrophomonas maltophilia group
unclassified Stenotrophomonas
unclassified Streptomyces
unclassified Symbiopectobacterium
unclassified Synechococcus
unclassified Synechocystis
unclassified Tardiphaga
unclassified Tolypothrix
unclassified Treponema
unclassified Trichocoleus
unclassified Tychonema
unclassified Variovorax
unclassified Xanthobacter
unclassified Yimella
unclassified Yonghaparkia
uncultured Actinomycetes bacterium
uncultured Aeromonas sp.
uncultured Agrobacterium sp.
uncultured Alcaligenes sp.
uncultured Alphaproteobacteria bacterium
uncultured Amorphomonas sp.
uncultured Anabaena sp.
uncultured Anaerotruncus sp.
uncultured Antarctic bacterium
uncultured Aquaspirillum sp.
uncultured Azoarcus sp.
uncultured Azohydromonas sp.
uncultured Azonexaceae bacterium
uncultured Azonexus sp.
uncultured Azorhizobium sp.
uncultured Azospira sp.
uncultured Azospirillum sp.
uncultured Azotobacter sp.
uncultured Azovibrio sp.
uncultured Bacillus sp.
uncultured Beijerinckia sp.
uncultured Blautia sp.
uncultured Bradyrhizobium sp.
uncultured Burkholderia sp.
uncultured Calothrix sp.
uncultured Candidatus Competibacteraceae bacterium
uncultured Celerinatantimonas sp.
uncultured Chlorobia bacterium
uncultured Chroococcales cyanobacterium
uncultured Clostridium sp.
uncultured Coprococcus sp.
uncultured Crocosphaera sp.
uncultured Cyanothece sp.
uncultured Dechloromonas sp.
uncultured Defluviicoccus sp.
uncultured Desulfatitalea sp.
uncultured Desulfobacteraceae bacterium
uncultured Desulfobacterium sp.
uncultured Desulfovibrio sp.
uncultured Desulfuromonas sp.
uncultured Dorea sp.
uncultured Ensifer sp.
uncultured Enterobacter sp.
uncultured Enterobacteriaceae bacterium
uncultured Epsilonproteobacteria bacterium
uncultured Eubacteriales bacterium
uncultured Eubacterium sp.
uncultured Firmicutes bacterium
uncultured Fischerella sp.
uncultured Frankia sp.
uncultured Gallionellaceae bacterium
uncultured Gammaproteobacteria bacterium
uncultured Gemmatimonadota bacterium
uncultured Geobacter sp.
uncultured Herbaspirillum sp.
uncultured Hydrogenophilales bacterium
uncultured Hyphomicrobiales bacterium
uncultured Insolitispirillum sp.
uncultured Klebsiella sp.
uncultured Kosakonia sp.
uncultured Magnetospirillum sp.
uncultured Martelella sp.
uncultured Mastigocladus sp.
uncultured Megasphaera sp.
uncultured Mesorhizobium sp.
uncultured Methylobacter sp.
uncultured Methylocella sp.
uncultured Methylocystis sp.
uncultured Methylosinus sp.
uncultured Microcoleus sp.
uncultured Nitrosomonadales bacterium
uncultured Nitrospirota bacterium
uncultured Nostoc sp.
uncultured Oscillatoriales cyanobacterium
uncultured Paenibacillus sp.
uncultured Paludibacter sp.
uncultured Pelomonas sp.
uncultured Phyllobacteriaceae bacterium
uncultured Pleomorphomonas sp.
uncultured Propionivibrio sp.
uncultured Pseudanabaena sp.
uncultured Pseudomonas sp.
uncultured Raoultella sp.
uncultured Rhizobium sp.
uncultured Rhodobacter sp.
uncultured Rhodocista sp.
uncultured Rhodocyclales bacterium
uncultured Rhodocyclus sp.
uncultured Rhodoplanes sp.
uncultured Richelia sp.
uncultured Ruminococcus sp.
uncultured Sideroxydans sp.
uncultured Sinorhizobium sp.
uncultured Skermanella sp.
uncultured Sporomusa sp.
uncultured Stigonematales cyanobacterium
uncultured Thiocapsa sp.
uncultured Tolypothrix sp.
uncultured Trichodesmium sp.
uncultured Verrucomicrobiota bacterium
uncultured Zoogloea sp.
uncultured ammonia-oxidizing bacterium
uncultured archaeon GZfos23H9
uncultured archaeon
uncultured bacterium BAC17H8
uncultured bacterium NR1600
uncultured bacterium NR1601
uncultured bacterium NR1602
uncultured bacterium NR1603
uncultured bacterium NR1604
uncultured bacterium NR1605
uncultured bacterium NR1606
uncultured bacterium NR1607
uncultured bacterium NR1608
uncultured bacterium NR1609
uncultured bacterium NR1610
uncultured bacterium NR1611
uncultured bacterium NR1612
uncultured bacterium NR1613
uncultured bacterium NR1614
uncultured bacterium NR1615
uncultured bacterium NR1616
uncultured bacterium NR1617
uncultured bacterium NR1618
uncultured bacterium NR1619
uncultured bacterium NR1620
uncultured bacterium NR1621
uncultured bacterium NR1622
uncultured bacterium NR1623
uncultured bacterium NR1624
uncultured bacterium NR1625
uncultured bacterium NR1626
uncultured bacterium NR1627
uncultured bacterium NR1628
uncultured bacterium NR1629
uncultured bacterium NR1630
uncultured bacterium NR1631
uncultured bacterium NR1632
uncultured bacterium NR1633
uncultured bacterium NR1634
uncultured bacterium NR1635
uncultured bacterium NR1636
uncultured bacterium NR1637
uncultured bacterium NR1638
uncultured bacterium NR1639
uncultured bacterium NR1640
uncultured bacterium NR1641
uncultured bacterium UPO35
uncultured bacterium
uncultured beta proteobacterium
uncultured crenarchaeote
uncultured cyanobacterium
uncultured delta proteobacterium
uncultured forest soil bacterium
uncultured heterocystous cyanobacterium
uncultured low G + C Gram-positive bacterium
uncultured marine bacterium MedDCM-OCT-S09-C247
uncultured marine bacterium
uncultured marine cyanobacterium
uncultured marine group II/III euryarchaeote AD1000_33_C07
uncultured marine group II/III euryarchaeote KM3_105_E02
uncultured marine group II/III euryarchaeote KM3_109_G01
uncultured marine group II/III euryarchaeote KM3_198_E02
uncultured marine group II/III euryarchaeote SAT1000_05_B02
uncultured marine group II/III euryarchaeote SAT1000_05_B04
uncultured marine microorganism
uncultured marine organism
uncultured methanogenic archaeon
uncultured methanotrophic bacterium
uncultured microorganism SN-10
uncultured microorganism SN-12
uncultured microorganism SN-13
uncultured microorganism SN-16
uncultured microorganism SN-17
uncultured microorganism SN-2
uncultured microorganism SN-33
uncultured microorganism SN-40
uncultured microorganism SN-42
uncultured microorganism SN-4
uncultured microorganism SN-8
uncultured microorganism
uncultured nitrogen-fixing bacterium A10
uncultured nitrogen-fixing bacterium A1
uncultured nitrogen-fixing bacterium A25
uncultured nitrogen-fixing bacterium A2
uncultured nitrogen-fixing bacterium A37
uncultured nitrogen-fixing bacterium A39
uncultured nitrogen-fixing bacterium A47
uncultured nitrogen-fixing bacterium A5
uncultured nitrogen-fixing bacterium A7
uncultured nitrogen-fixing bacterium B10
uncultured nitrogen-fixing bacterium B12
uncultured nitrogen-fixing bacterium B13
uncultured nitrogen-fixing bacterium B18
uncultured nitrogen-fixing bacterium B1
uncultured nitrogen-fixing bacterium B21
uncultured nitrogen-fixing bacterium B25
uncultured nitrogen-fixing bacterium B30
uncultured nitrogen-fixing bacterium B34
uncultured nitrogen-fixing bacterium B3
uncultured nitrogen-fixing bacterium B55
uncultured nitrogen-fixing bacterium B9
uncultured nitrogen-fixing bacterium C1
uncultured nitrogen-fixing bacterium C20
uncultured nitrogen-fixing bacterium C5
uncultured nitrogen-fixing bacterium
uncultured nitrogen-fixing eubacterium ‘Canada Stream 13’
uncultured nitrogen-fixing eubacterium ‘Canada Stream 4’
uncultured nitrogen-fixing eubacterium ‘Dry Pond 10’
uncultured nitrogen-fixing eubacterium ‘Ice Aggregate 11b’
uncultured nitrogen-fixing eubacterium ‘Ice aggregate 1’
uncultured nitrogen-fixing eubacterium ‘Ice aggregate 1.1’
uncultured nitrogen-fixing eubacterium ‘Ice aggregate 11’
uncultured nitrogen-fixing eubacterium ‘Ice aggregate 2.6’
uncultured nitrogen-fixing eubacterium ‘Ice aggregate 3.1’
uncultured nitrogen-fixing eubacterium ‘Ice aggregate 3.4’
uncultured nitrogen-fixing eubacterium ‘Ice aggregate 3.5’
uncultured nitrogen-fixing eubacterium ‘Ice aggregate 3.6’
uncultured nitrogen-fixing eubacterium ‘Ice aggregate 3.9’
uncultured nitrogen-fixing eubacterium ‘Ice aggregate 4.2’
uncultured nitrogen-fixing eubacterium ‘Ice aggregate a11’
uncultured organism
uncultured prokaryote
uncultured proteobacterium
uncultured soil bacterium A1
uncultured soil bacterium B2
uncultured soil bacterium C1
uncultured soil bacterium D2
uncultured soil bacterium E1
uncultured soil bacterium F3
uncultured soil bacterium G1
uncultured soil bacterium H5
uncultured soil bacterium I4
uncultured soil bacterium
uncultured temperate forest soil bacterium CF004a
uncultured temperate forest soil bacterium CF018a
uncultured temperate forest soil bacterium CF049
uncultured temperate forest soil bacterium CF051
uncultured temperate forest soil bacterium CF052
uncultured temperate forest soil bacterium CFY01
uncultured temperate forest soil bacterium VF003
uncultured temperate forest soil bacterium VF009
uncultured temperate forest soil bacterium
unidentified actinomycete L5
unidentified marine bacterial clone HT1901
unidentified marine bacterial clone HT1902
unidentified marine bacterial clone HT1903
unidentified marine bacterial clone HT1904
unidentified marine bacterial clone HT1905
unidentified marine bacterial clone HT1906
unidentified marine bacterial clone HT1907
unidentified marine bacterial clone HT1908
unidentified marine bacterial clone HT1909
unidentified marine eubacterium clone AO1102
unidentified marine eubacterium clone AO1104
unidentified marine eubacterium clone AO1113
unidentified marine eubacterium clone BH1132
unidentified marine eubacterium clone BT1101
unidentified marine eubacterium clone BT1118
unidentified marine eubacterium clone HT1103
unidentified marine eubacterium clone HT1150
unidentified marine eubacterium clone HT1169
unidentified marine eubacterium clone HT1177
unidentified marine eubacterium clone HT1192
unidentified marine eubacterium clone HT1193
unidentified marine eubacterium clone HT1195
unidentified marine eubacterium clone HT1196
unidentified marine eubacterium clone HT1197
unidentified marine eubacterium clone HT1198
unidentified marine eubacterium clone HT1199
unidentified marine eubacterium clone HT1200
unidentified marine eubacterium clone HT1201
unidentified marine eubacterium clone HT1202
unidentified marine eubacterium clone HT1203
unidentified marine eubacterium clone HT1204
unidentified marine eubacterium clone HT1205
unidentified marine eubacterium clone PO3120
unidentified marine eubacterium clone PO3133
unidentified marine eubacterium clone PO3135
unidentified marine eubacterium clone PO3137
unidentified marine eubacterium
unidentified marine proteobacterium ‘UnkAssoc 1’
unidentified marine proteobacterium ‘UnkAssoc 2’
unidentified marine proteobacterium ‘UnkAssoc 4’
unidentified nitrogen-fixing bacteria

In some embodiments, an archaea can fix nitrogen. In some embodiments, the consortium of bacteria may further comprise an archaea. In some embodiments, the consortium of bacteria further comprises an archaea capable of fixing nitrogen.

In some embodiments, one or more bacterial species of the bacterial consortia may be capable of fixing nitrogen. In some cases, one or more species of the bacterial consortia may facilitate or enhance the ability of other bacteria to fix nitrogen. The bacteria which fix nitrogen and the bacteria which enhance the ability of other bacteria to fix nitrogen may be the same or different. In some examples, a bacterial strain may be able to fix nitrogen when in combination with a different bacterial strain, or in a certain bacterial consortia, but may be unable to fix nitrogen in a monoculture.

### Growth and Survival

A nitrogen-fixing organism described herein can use a carbon source and a source of nitrogen to grow and live. Carbon sources include without limitation malic acid, acetate, sugars such as glucose or sucrose, alcohols, starch, alkanes, carbon dioxide, methane, methanol, methylamine, trimethylamine, and mixtures thereof. Carbon sources can be derived from a range of industrial or natural sources. Nitrogen sources may include without limitation, ambient air, enriched air, ammonium, ammonia, nitrates, or nitrites. In some embodiments, the nitrogen source may comprise N₂ at atmospheric concentrations. In some embodiments, the nitrogen source may comprise N₂ at about 100,000, 150,000, 200,000, 250,000, 300,000, 350,000, 400,000, 450,000, 500,000, 550,000, 600,000, 650,000, 700,000, 750,000, 760,000, 770,000, 780,000, 780,840, 790,000, 800,000, 810,000, 820,000, or 830,000 parts per million (ppm). In some embodiments, the nitrogen source may comprise N₂ at a concentration of at least about 100,000 ppm. In some embodiments, the nitrogen source may comprise N₂ at a concentration of more than about 700,000 ppm.

Nitrogen-fixing bacteria can survive in growth medium lacking any exogenous form of nitrogen beyond the N₂ which is found in the air. In some embodiments, cultivation of N₂ fixing bacteria may comprise standard bacterial culture conditions. In some embodiments, additional components may improve the survival of nitrogen fixing bacteria. In certain embodiments, a liquid or solid nutrient media may contain nutrients suitable to sustain viability of nitrogen fixing bacteria over time. In some embodiments, nitrogen fixation is anaerobic. In certain embodiments, nitrogen fixation may occur in aerobic environments. Oxygen sources may include but are not limited to air, enriched air, O₂ from fractional distillation, pressure swing adsorption, an oxygen concentrator, electrolysis of water and liquid O₂.

In some embodiments, cultivation of N₂ fixing bacteria is enhanced or improved with the addition of additives. In some embodiments, the additives may influence the ability of bacteria to fix nitrogen. For example, it is known that molybdenum and iron are part of nitrogenase, an enzyme that catalyzes the reduction of N₂. It is also known that boron is necessary for the nodulation of Rhizobium and that zinc and manganese are part of the superoxide dismutase, an enzyme that protects against oxidative stress. In some cases, copper is part of a special cytochrome C which aides in respiration during the low oxygen conditions in which nitrogen fixation takes place. In some embodiments, nickel is a cofactor of hydrogenase, an enzyme that processes hydrogen generated in nitrogen fixation and cobalt is part of cobalamin, an essential cofactor for some enzymes indirectly involved in nitrogen fixation.

In some embodiments, cultivation of nitrogen-fixing bacteria can include Na₂HPO₄, CaCl₂ 2H₂O, KH₂PO₄, MgSO₄·7H₂O, NaCl, FeCl₃, Na₂MoO₄, and sucrose. In some embodiments, cultivation of nitrogen-fixing bacteria can include (per liter) 25 g Na₂HPO₄, 0.1 g CaCl₂-2H₂O, 3 g KH₂PO₄, 0.25 g MgSO₄ 7H₂O, lg NaCl, 2.9 mg FeCl₃, 0.25 mg Na₂MoO₄ 2H₂O, and 20 g sucrose. In some embodiments, growth medium may comprise minimal medium supplemented with 50 mL of 200 mM glutamine per liter.

### Measuring N₂ Fixation

Methods for measuring N₂ fixation are known in the art, for example, as disclosed by, White et al. (2020. Limnology and Oceanography Methods. 18:129-147), which is incorporated herein by reference in its entirety.

In some embodiments, measuring N₂ fixation may comprise measuring the concentration of N₂ fixed by the consortium. In some embodiments, measuring N₂ fixation may comprise measuring the concentration of N₂ in a closed system by determining the change in concentration of N₂ at an influx as compared to the concentration of N₂ at an outflux. In some embodiments, measuring N₂ fixation may comprise measuring the concentration of N₂ in a closed system by determining the change in concentration of N₂ at one earlier time point as compared to the concentration of N₂ at one or more later time points. In some embodiments, measuring N₂ concentration in air may comprise using gas chromatography. In some embodiments, measuring N₂ concentration in the air may comprise using thermal conductivity detector gas chromatography or some variation thereof. In some embodiments, N₂ concentration is measured by detecting the ions formed by combustion of a sample in a hydrogen or air flame. In some embodiments, an instrument capable of gas chromatography can be modified to suit various environments. In certain embodiments, features such as column length of the gas chromatography instrument, analysis cycle time, or sample size can be modified.

In certain embodiments, N₂ fixation may be determined by measuring total nitrogen in soil, compost, or liquid samples. In some embodiments, a carbon-nitrogen elemental analyzer may be used to measure total nitrogen in samples. In some embodiments, a carbon-nitrogen elemental analyzer may be used to perform a combustion analysis. In some embodiments, a carbon-nitrogen elemental analyzer may be used to measure total nitrogen in samples using Dumas combustion method. In some embodiments, a carbon-nitrogen elemental analyzer may use infrared absorption and thermal conductivity to measure combustion gases within a metallic sample. In some embodiments, a sample (nonlimiting examples of which include a solid or compost) may be freeze dried to remove moisture. In some embodiments, the freeze dried samples may be converted into fine powders, encapsulated, and dispensed into the analyzer. For example, suitable media are described in Mkohonza et al., 2020 and Kowanlenko, 2001, which are incorporated herein by reference in their entirety.

In some embodiments, measuring N₂ fixation may comprise using nitrogen free culture media. In some embodiments, N₂ fixation may be measured using a bio-assay. In certain embodiments, bacteria may be grown in media lacking N₂ so that atmospheric N₂ is the sole nitrogen source. In some embodiments, measuring bacteria grown on or in nitrogen-free media by methods such as weight or optical density (for example, OD₆₀₀) of bacteria suspended in culture may provide a quantitative determination of nitrogen fixation.

In some embodiments, measuring N₂ consumption may comprise using a reactor. In some embodiments nitrogen fixation is measured via a single fermentative assay. In some embodiments, nitrogen excretion is measured with a single fermentative assay.

In some cases, nitrogen fixation may be measured using acetylene reduction assay (ARA). In some embodiments, an ARA measures nitrogenase activity. The nitrogenase enzyme has been shown to not be very specific, such that it is not only able to reduce N₂ to NH₄, but may also nonspecifically reduce other chemical compounds such as 2H⁺ to H₂; N₂O to N₂; H₂O and CN to CH₄ and NH₃. In some instances, nitrogenase is also capable of reducing C₂H₂ to C₂H₄. In some embodiments of this disclosure, an acetylene reduction test may determine the speed at which a given weight of sample is able to reduce the C₂H₂ to C₂H₄ as an indirect measure of the activity of the nitrogenase enzyme. In some embodiments, the ARA may use gas chromatography to measure acetylene. In some embodiments, ARA can be performed in high throughput plates of microtube arrays. In certain embodiments, ARA can be performed with live plants and plant tissues. The media formulation and media oxygen concentration can be varied in ARA assays.

In some embodiments, measuring N₂ fixation may comprise using labeled N₂. In some embodiments, N₂ fixation may comprise using radiolabeled elements. In some embodiments, measuring N₂ fixation may comprise using radiolabeled elements such as ¹⁵N, ¹³N, ¹⁴C, ¹³C, or ³H. In some embodiments, the radiolabeled elements may be used to measure ¹⁵N₂, ¹⁵NH₃, or ¹⁵NH₄. In some embodiments, tracing the incorporation of stable isotope-labeled N₂ gas (¹⁵N₂) into particulate matter may be used to measure N₂ fixation. In certain embodiments, the consortium of bacteria may be incubated with ¹⁵N₂. In some embodiments, bacteria can be incubated with radiolabeled ¹⁵N₂ by introducing labeled ¹⁵N₂ into the headspace of a reactor, incubator, or other closed system. In some embodiments, radiolabeled ¹⁵N₂ may be introduced to soil samples comprising a consortium of bacteria. In some embodiments, radiolabeled ¹⁵N₂ is measured in the consortium of bacteria. In some embodiments, radiolabeled ¹⁵N₂ is measured in lipids or another cellular component of the consortium of bacteria. In some embodiments, measuring radiolabeled ¹⁵N₂ may comprise using gas chromatography. In some embodiments, measuring radiolabeled ¹⁵N₂ may comprise using flame ionization detection gas chromatography. In some embodiments, measuring radiolabeled ¹⁵N₂ may comprise using mass spectrometry. In some embodiments, determining nitrogen fixation of a consortium of bacteria incubated with radiolabeled ¹⁵N₂ may be compared to a similar sample that was not incubated with radiolabeled ¹⁵N₂ or to a sample that lacks the capacity to fix ¹⁵N₂.

In some embodiments, measuring N₂ fixation may comprise measuring the amount of radiolabeled material in a composition. In some embodiments, measuring N₂ fixation may comprise measuring the amount of radiolabeled material in a dry weight composition. In some embodiments, measuring N₂ fixation may comprise measuring gas using a reactor.

In certain embodiments, the bacterial consortium disclosed herein fixes N₂ at a rate of about 0.6 umol/gDW per day to about 2.8 umol/gDW per day. In certain embodiments, the bacterial consortium disclosed herein fixes N₂ at a rate of about 0.6 umol/gDW per day to about 0.8 umol/gDW per day, about 0.6 umol/gDW per day to about 1 umol/gDW per day, about 0.6 umol/gDW per day to about 1.2 umol/gDW per day, about 0.6 umol/gDW per day to about 1.4 umol/gDW per day, about 0.6 umol/gDW per day to about 1.6 umol/gDW per day, about 0.6 umol/gDW per day to about 1.8 umol/gDW per day, about 0.6 umol/gDW per day to about 2 umol/gDW per day, about 0.6 umol/gDW per day to about 2.2 umol/gDW per day, about 0.6 umol/gDW per day to about 2.4 umol/gDW per day, about 0.6 umol/gDW per day to about 2.6 umol/gDW per day, about 0.6 umol/gDW per day to about 2.8 umol/gDW per day, about 0.8 umol/gDW per day to about 1 umol/gDW per day, about 0.8 umol/gDW per day to about 1.2 umol/gDW per day, about 0.8 umol/gDW per day to about 1.4 umol/gDW per day, about 0.8 umol/gDW per day to about 1.6 umol/gDW per day, about 0.8 umol/gDW per day to about 1.8 umol/gDW per day, about 0.8 umol/gDW per day to about 2 umol/gDW per day, about 0.8 umol/gDW per day to about 2.2 umol/gDW per day, about 0.8 umol/gDW per day to about 2.4 umol/gDW per day, about 0.8 umol/gDW per day to about 2.6 umol/gDW per day, about 0.8 umol/gDW per day to about 2.8 umol/gDW per day, about 1 umol/gDW per day to about 1.2 umol/gDW per day, about 1 umol/gDW per day to about 1.4 umol/gDW per day, about 1 umol/gDW per day to about 1.6 umol/gDW per day, about 1 umol/gDW per day to about 1.8 umol/gDW per day, about 1 umol/gDW per day to about 2 umol/gDW per day, about 1 umol/gDW per day to about 2.2 umol/gDW per day, about 1 umol/gDW per day to about 2.4 umol/gDW per day, about 1 umol/gDW per day to about 2.6 umol/gDW per day, about 1 umol/gDW per day to about 2.8 umol/gDW per day, about 1.2 umol/gDW per day to about 1.4 umol/gDW per day, about 1.2 umol/gDW per day to about 1.6 umol/gDW per day, about 1.2 umol/gDW per day to about 1.8 umol/gDW per day, about 1.2 umol/gDW per day to about 2 umol/gDW per day, about 1.2 umol/gDW per day to about 2.2 umol/gDW per day, about 1.2 umol/gDW per day to about 2.4 umol/gDW per day, about 1.2 umol/gDW per day to about 2.6 umol/gDW per day, about 1.2 umol/gDW per day to about 2.8 umol/gDW per day, about 1.4 umol/gDW per day to about 1.6 umol/gDW per day, about 1.4 umol/gDW per day to about 1.8 umol/gDW per day, about 1.4 umol/gDW per day to about 2 umol/gDW per day, about 1.4 umol/gDW per day to about 2.2 umol/gDW per day, about 1.4 umol/gDW per day to about 2.4 umol/gDW per day, about 1.4 umol/gDW per day to about 2.6 umol/gDW per day, about 1.4 umol/gDW per day to about 2.8 umol/gDW per day, about 1.6 umol/gDW per day to about 1.8 umol/gDW per day, about 1.6 umol/gDW per day to about 2 umol/gDW per day, about 1.6 umol/gDW per day to about 2.2 umol/gDW per day, about 1.6 umol/gDW per day to about 2.4 umol/gDW per day, about 1.6 umol/gDW per day to about 2.6 umol/gDW per day, about 1.6 umol/gDW per day to about 2.8 umol/gDW per day, about 1.8 umol/gDW per day to about 2 umol/gDW per day, about 1.8 umol/gDW per day to about 2.2 umol/gDW per day, about 1.8 umol/gDW per day to about 2.4 umol/gDW per day, about 1.8 umol/gDW per day to about 2.6 umol/gDW per day, about 1.8 umol/gDW per day to about 2.8 umol/gDW per day, about 2 umol/gDW per day to about 2.2 umol/gDW per day, about 2 umol/gDW per day to about 2.4 umol/gDW per day, about 2 umol/gDW per day to about 2.6 umol/gDW per day, about 2 umol/gDW per day to about 2.8 umol/gDW per day, about 2.2 umol/gDW per day to about 2.4 umol/gDW per day, about 2.2 umol/gDW per day to about 2.6 umol/gDW per day, about 2.2 umol/gDW per day to about 2.8 umol/gDW per day, about 2.4 umol/gDW per day to about 2.6 umol/gDW per day, about 2.4 umol/gDW per day to about 2.8 umol/gDW per day, or about 2.6 umol/gDW per day to about 2.8 umol/gDW per day.

In certain embodiments, the bacterial consortium disclosed herein fixes N₂ at a rate of about 0.6 umol/gDW per day, about 0.8 umol/gDW per day, about 1 umol/gDW per day, about 1.2 umol/gDW per day, about 1.4 umol/gDW per day, about 1.6 umol/gDW per day, about 1.8 umol/gDW per day, about 2 umol/gDW per day, about 2.2 umol/gDW per day, about 2.4 umol/gDW per day, about 2.6 umol/gDW per day, or about 2.8 umol/gDW per day. In certain embodiments, the bacterial consortium disclosed herein fixes N₂ at a rate of at least about 0.6 umol/gDW per day, about 0.8 umol/gDW per day, about 1 umol/gDW per day, about 1.2 umol/gDW per day, about 1.4 umol/gDW per day, about 1.6 umol/gDW per day, about 1.8 umol/gDW per day, about 2 umol/gDW per day, about 2.2 umol/gDW per day, about 2.4 umol/gDW per day, or about 2.6 umol/gDW per day. In certain embodiments, the bacterial consortium disclosed herein fixes N₂ at a rate of at most about 0.8 umol/gDW per day, about 1 umol/gDW per day, about 1.2 umol/gDW per day, about 1.4 umol/gDW per day, about 1.6 umol/gDW per day, about 1.8 umol/gDW per day, about 2 umol/gDW per day, about 2.2 umol/gDW per day, about 2.4 umol/gDW per day, about 2.6 umol/gDW per day, or about 2.8 umol/gDW per day.

### Nitrogen Fixing Methanotrophs

In some embodiments, disclosed herein are bacteria capable of consuming methane and fixing nitrogen. In some embodiments of the consortium of bacteria disclosed herein, a species capable of consuming methane may also be capable of fixing nitrogen. In some embodiments, bacteria capable of consuming methane and capable of fixing nitrogen are listed in Table 4. In some embodiments, the consortium of bacteria disclosed herein comprises a bacteria listed in Table 1A, Table 1B, Table 1C, Table 1D, Table 3, Table 4, or a combination thereof.

In some embodiments, the consortium of bacteria may comprise at least one or more diazotroph that is also a methanotroph. In certain embodiments, the bacteria that are capable of consuming methane and fixing nitrogen may be phylogenetically diverse. In some embodiments, the consortium of bacteria comprises at least 1, 2, 3, 4, or more different bacterial phyla that are capable of consuming methane and fixing nitrogen. In certain non-limiting embodiments, a bacteria capable of consuming methane fixing nitrogen may belong to, for example, the phyla Pseudomonadota. In certain embodiments, the consortium of bacteria may comprise at least 1, 2, 3, 4, 5, 6, 7, 8, 9, 10, 15, 20, 30, or more different bacterial order. Non-limiting examples of orders for which bacteria in the consortium capable of consuming methane fixing nitrogen may belong to Hyphomicrobiales or Rhodospirillales. In certain embodiments, the consortium of bacteria may comprise at least 1, 2, 3, 4, 5, 6, 7, 8, 9, 10, 15, 20, 30, 40, 50, or more different bacterial family. In certain embodiments, a bacteria capable of consuming methane and fixing nitrogen may belong, in a non-limiting example, to the family Methylocystaceae, Beijerinckiaceae, Nitrobacteraceae, or Bradyrhizobiaceae. In certain embodiments, the consortium of bacteria may comprise at least 1, 2, 3, 4, 5, 6, 7, 8, 9, 10, 15, 20, 30, 40, 50, or more different bacterial genera. In certain non-limiting embodiments, a bacteria capable of consuming methane and fixing nitrogen may belong, for example, to the genera Azospirillum, Amycolatopsis, Acidiphilium, Burkholderia, Methylosinus, Methylocystis, Methylopila, Mesorhizobium Rhodococcus, Pseudonocardia, Pseudomonas, Streptomyces, Nocardia, Mycolicibacterium, or Mycobacterium. In some embodiments, the consortium of bacteria may comprise 1, 2, 3, 4, 5, 6, 7, 8, 9, 10, 11, 12, 13, 14, 15, 16, 17, 18, 19, 20, 25, 30, 35, 40, 45, 50, 55, 60, or more species that are capable of consuming methane and fixing nitrogen.

In various embodiments, a species that is capable of consuming methane and of fixing nitrogen may express nitrogenase and methane monooxygenase or methyl coenzyme A reductase. In some embodiments, the consortium of bacteria may comprise one or more species from Table 4. In some embodiments, the consortium of bacteria may comprise 1, 2, 3, 4, 5, 6, 7, 8, 9, 10, or more species from Table 4. In certain embodiments, the consortium of bacteria may comprise one or more species from Table 4 in combination with other bacteria that may consume methane only, fix nitrogen only, or do neither consume methane nor fix nitrogen. In various embodiments, the consortium of bacteria may comprise only bacteria listed in Table 4.

TABLE 4
Species containing both mmo and nifH gene homologs

Species
Acetivibrio cellulolyticus
Acetivibrio clariflavus
Acetoanaerobium sticklandii
Acetobacterium bakii
Acetobacterium dehalogenans
Acetobacterium fimetarium
Acetobacterium paludosum
Acetobacterium sp. KB-1
Acetobacterium tundrae
Acetobacterium wieringae
Acetobacterium woodii
Acetobacterium
Acetohalobium arabaticum
Acetonema longum
Achromobacter denitrificans
Achromobacter insolitus
Acidaminobacter sp. JC074
Acidianus brierleyi
Acidiferrobacter sp. SPIII_3
Acidiferrobacter thiooxydans
Acidihalobacter aeolianus
Acidihalobacter prosperus
Acidiphilium multivorum AIU301
Acidiphilium multivorum
Acidiphilium sp. 21-68-69
Acidiphilium sp. 34-64-41
Acidiphilium sp. 37-64-53
Acidiphilium sp. 37-67-22
Acidiphilium sp. PM
Acidiphilium
Acidithiobacillus ferridurans
Acidithiobacillus ferriphilus
Acidithiobacillus ferrivorans SS3
Acidithiobacillus ferrivorans
Acidithiobacillus ferrooxidans ATCC 23270
Acidithiobacillus ferrooxidans ATCC 53993
Acidithiobacillus ferrooxidans
Acidithiobacillus sp. ‘AMD consortium’
Acidithiobacillus
Acidobacteriota bacterium
Acidovorax caeni
Acidovorax delafieldii
Acidovorax sp. JHL-3
Acinetobacter baumannii
Acinetobacter haemolyticus
Acinetobacter pittii
Acinetobacter sp. V2
Actinacidiphila alni
Actinacidiphila bryophytorum
Actinacidiphila guanduensis
Actinacidiphila soli
Actinoallomurus bryophytorum
Actinomycetospora callitridis
Actinomycetospora chiangmaiensis
Actinomycetospora chibensis
Actinomycetospora cinnamomea
Actinomycetospora corticicola
Actinomycetospora endophytica
Actinomycetospora lutea
Actinomycetospora soli
Actinomycetospora sp. DW7H6
Actinomycetospora sp. TBRC 11914
Actinomycetospora straminea
Actinomycetospora succinea
Actinomycetota bacterium
Actinophytocola oryzae
Actinoplanes atraurantiacus
Actinoplanes bogorensis
Actinoplanes brasiliensis
Actinoplanes campanulatus
Actinoplanes deccanensis
Actinoplanes globisporus
Actinoplanes lobatus
Actinoplanes regularis
Actinoplanes sp. ATCC 53533
Actinoplanes sp. KI2
Actinoplanes sp. LAM7112
Actinoplanes sp. Pm04-4
Actinoplanes sp. TBRC 11911
Actinoplanes subtropicus
Actinoplanes utahensis
Actinoplanes xinjiangensis
Acuticoccus sediminis
Aeromonas allosaccharophila
Aeromonas caviae
Aeromonas dhakensis
Aeromonas hydrophila
Aeromonas piscicola
Aeromonas simiae
Aeromonas sp. L_1B5_3
Aeromonas veronii
Aeromonas
Afipia felis
Agreia sp. COWG
Aliarcobacter butzleri
Aliarcobacter cibarius
Alkaliflexus imshenetskii
Alkalilimnicola ehrlichii
Alkaliphilus metalliredigens
Alkaliphilus oremlandii
Alkaliphilus peptidifermentans
Alkaliphilus pronyensis
Alkaliphilus serpentinus
Alkaliphilus transvaalensis
Alkalispirochaeta alkalica
Alkalispirochaeta americana
Alkalitalea saponilacus
Allonocardiopsis opalescens
Altererythrobacter sp. XM-24bin4
Amaricoccus solimangrovi
Aminiphilus circumscriptus
Aminipila butyrica
Aminipila terrae
Amphritea atlantica
Amphritea japonica
Amphritea pacifica
Amycolatopsis acidicola
Amycolatopsis acididurans
Amycolatopsis acidiphila
Amycolatopsis alba
Amycolatopsis australiensis
Amycolatopsis benzoatilytica
Amycolatopsis bullii
Amycolatopsis camponoti
Amycolatopsis circi
Amycolatopsis deserti
Amycolatopsis eburnea
Amycolatopsis echigonensis
Amycolatopsis jejuensis
Amycolatopsis kentuckyensis
Amycolatopsis lexingtonensis
Amycolatopsis mediterranei
Amycolatopsis methanolica
Amycolatopsis niigatensis
Amycolatopsis orientalis
Amycolatopsis pittospori
Amycolatopsis pretoriensis
Amycolatopsis rhizosphaerae
Amycolatopsis rubida
Amycolatopsis saalfeldensis
Amycolatopsis sacchari
Amycolatopsis sp. AA4
Amycolatopsis sp. ATCC 39116
Amycolatopsis sp. BJA-103
Amycolatopsis sp. CA-126428
Amycolatopsis sp. CA-128772
Amycolatopsis sp. FDAARGOS 1241
Amycolatopsis sp. FU40
Amycolatopsis sp. HUAS 11-8
Amycolatopsis sp. Hca4
Amycolatopsis sp. M39
Amycolatopsis sp. OK19-0408
Amycolatopsis sp. Poz14
Amycolatopsis sp. SID8362
Amycolatopsis sp. WQ 127309
Amycolatopsis sulphurea
Amycolatopsis thermoflava
Amycolatopsis tolypomycina
Amycolatopsis vancoresmycina
Amycolatopsis vastitatis
Amycolatopsis xylanica
Amycolatopsis
Anabaena sp. UHCC 0204
Anabaena sp. UHCC 0253
Anaeroarcus burkinensis
Anaerobacterium chartisolvens
Anaerocolumna chitinilytica
Anaerocolumna jejuensis
Anaerocolumna xylanovorans
Anaeromicrobium sediminis
Anaeromyxobacter dehalogenans
Anaeromyxobacter diazotrophicus
Anaeromyxobacter oryzae
Anaeromyxobacter paludicola
Anaeromyxobacter sp. Fw109-5
Anaeromyxobacter sp. K
Anaeromyxobacter sp. PSR-1
Anaeromyxobacter sp. SG22
Anaeropeptidivorans aminofermentans
Anaerophaga thermohalophila
Anaerosalibacter massiliensis
Anaerospora hongkongensis
Anaerosporobacter mobilis
Anaerosporomusa subterranea
Anaerotignum neopropionicum
Anaerotignum propionicum
Anaerotruncus colihominis
Aneurinibacillus tyrosinisolvens
Antarctobacter heliothermus
Aquabacterium sp. CECT 9606
Aquabacterium sp. NJ1
Aquabacterium sp. OR-4
Arcobacter acticola
Arcobacter caeni
Arcobacter defluvii
Arcobacter ellisii
Arcobacter sp. ARW1-2F2
Arcobacter suis
Arcobacter venerupis
Arcobacteraceae
Arcticibacter tournemirensis
Arenibaculum pallidiluteum
Aromatoleum diolicum
Aromatoleum evansii
Aromatoleum toluclasticum
Aromatoleum tolulyticum
Aromatoleum toluolicum
Aromatoleum toluvorans
Arthrobacter sp. AG258
Arthrobacter sp. Bi26
Asanoa hainanensis
Asanoa iriomotensis
Aurantimonas marina
Azoarcus communis
Azoarcus indigens
Azoarcus olearius
Azoarcus sp. Aa7
Azoarcus sp. DD4
Azoarcus sp. KH32C
Azoarcus sp. TTM-91
Azoarcus sp.
Azonexus fungiphilus
Azonexus hydrophilus
Azonexus sp. R2A-61
Azospira inquinata
Azospira oryzae
Azospira restricta
Azospira sp. I09
Azospira sp. I13
Azospira
Azospirillum agricola
Azospirillum argentinense
Azospirillum baldaniorum
Azospirillum brasilense
Azospirillum canadense
Azospirillum cavernae
Azospirillum doebereinerae
Azospirillum fermentarium
Azospirillum formosense
Azospirillum halopraeferens
Azospirillum humicireducens
Azospirillum lipoferum
Azospirillum melinis
Azospirillum oleiclasticum
Azospirillum oryzae
Azospirillum palustre
Azospirillum picis
Azospirillum ramasamyi
Azospirillum rugosum
Azospirillum soli
Azospirillum sp. 412522
Azospirillum sp. A1-3
Azospirillum sp. B21
Azospirillum sp. B506
Azospirillum sp. B510
Azospirillum sp. Sh1
Azospirillum sp. TSA2s
Azospirillum sp. TSA6c
Azospirillum sp. TSH100
Azospirillum sp. TSH20
Azospirillum sp. TSH58
Azospirillum sp. TSH64
Azospirillum sp. TSH7
Azospirillum sp. TSO22-1
Azospirillum sp. TSO35-2
Azospirillum sp. TSO5
Azospirillum thermophilum
Azospirillum thiophilum
Azospirillum
Azovibrio restrictus
Bacillus smithii
Bacillus sp. 1NLA3E
Bacillus sp. EB600
Baekduia sp. 0141_2
Batillaria attramentaria
Bdellovibrio bacteriovorus
Bdellovibrio sp. SKB1291214
Beggiatoa leptomitoformis
Beijerinckia indica subsp. indica ATCC 9039
Beijerinckiaceae bacterium
Betaproteobacteria bacterium HGW-Betaproteobacteria-11
Betaproteobacteria bacterium HGW-Betaproteobacteria-13
Betaproteobacteria bacterium HGW-Betaproteobacteria-1
Betaproteobacteria bacterium
Brachyspira alvinipulli
Brachyspira hampsonii
Brachyspira hyodysenteriae
Brachyspira innocens
Brachyspira intermedia
Brachyspira pilosicoli
Brachyspira suanatina
Bradyrhizobiaceae bacterium
Bradyrhizobium aeschynomenes
Bradyrhizobium brasilense
Bradyrhizobium diazoefficiens USDA 110
Bradyrhizobium diazoefficiens
Bradyrhizobium diversitatis
Bradyrhizobium forestalis
Bradyrhizobium frederickii
Bradyrhizobium icense
Bradyrhizobium ivorense
Bradyrhizobium japonicum
Bradyrhizobium macuxiense
Bradyrhizobium manausense
Bradyrhizobium neotropicale
Bradyrhizobium niftali
Bradyrhizobium oligotrophicum
Bradyrhizobium ottawaense
Bradyrhizobium rifense
Bradyrhizobium sacchari
Bradyrhizobium shewense
Bradyrhizobium sp. 14AB
Bradyrhizobium sp. 6(2017)
Bradyrhizobium sp. A19
Bradyrhizobium sp. AC87j1
Bradyrhizobium sp. CCBAU 45389
Bradyrhizobium sp. CCBAU 51765
Bradyrhizobium sp. CCBAU 53340
Bradyrhizobium sp. CCBAU 53421
Bradyrhizobium sp. CCGB12
Bradyrhizobium sp. I71
Bradyrhizobium sp. LCT2
Bradyrhizobium sp. Leo121
Bradyrhizobium sp. Leo170
Bradyrhizobium sp. MOS001
Bradyrhizobium sp. NAS96.2
Bradyrhizobium sp. SEMIA
Bradyrhizobium sp. WD16
Bradyrhizobium sp. WSM2254
Bradyrhizobium sp. WSM3983
Bradyrhizobium sp.
Bradyrhizobium uaiense
Bradyrhizobium zhanjiangense
Bradyrhizobium
Breznakibacter xylanolyticus
Breznakiella homolactica
Burkholderia aenigmatica
Burkholderia ambifaria
Burkholderia anthina
Burkholderia arboris
Burkholderia cepacia
Burkholderia contaminans
Burkholderia glumae
Burkholderia lata
Burkholderia plantarii
Burkholderia pyrrocinia
Burkholderia sp. Ac-20345
Burkholderia sp. Ac-20349
Burkholderia sp. Ac-20392
Burkholderia sp. JP2-270
Burkholderia sp. LMG 13014
Burkholderia sp. Se-20378
Burkholderia sp. lig30
Burkholderia stabilis
Burkholderia ubonensis MSMB22
Burkholderia vietnamiensis LMG 10929
Burkholderia vietnamiensis
Burkholderia
Burkholderiales bacterium PBB1
Caballeronia novacaledonica
Caballeronia sordidicola
Caballeronia temeraria
Caballeronia udeis
Caballeronia zhejiangensis
Caenispirillum bisanense
Caenispirillum salinarum
Caldicellulosiruptor acetigenus
Caldicellulosiruptor bescii
Caldicellulosiruptor danielii
Caldicellulosiruptor diazotrophicus
Caldicellulosiruptor kronotskyensis
Caldicellulosiruptor morganii
Caldicellulosiruptor naganoensis
Caldicellulosiruptor obsidiansis
Caldicellulosiruptor owensensis
Caldicellulosiruptor saccharolyticus
Caldicellulosiruptor
Caldicoprobacter faecalis
Caldinitratiruptor microaerophilus
Calditerrivibrio nitroreducens
Caloramator australicus
Caloranaerobacter sp. TR13
Campylobacter armoricus
Campylobacter coli
Campylobacter jejuni
Campylobacter ornithocola
Campylobacter volucris
Campylobacter
Candidatus Accumulibacter aalborgensis
Candidatus Accumulibacter cognatus
Candidatus Accumulibacter contiguus
Candidatus Accumulibacter phosphatis
Candidatus Brocadia sinica
Candidatus Competibacter denitrificans
Candidatus Competibacter phosphatis
Candidatus Contendobacter odensis
Candidatus Dactylopiibacterium carminicum
Candidatus Endoriftia persephone
Candidatus Formimonas warabiya
Candidatus Frankia alpina
Candidatus Frankia californiensis
Candidatus Koribacter versatilis
Candidatus Kryptonium thompsoni
Candidatus Kuenenia stuttgartiensis
Candidatus Macondimonas diazotrophica
Candidatus Magnetaquicoccus inordinatus
Candidatus Magnetobacterium casensis
Candidatus Marinarcus aquaticus
Candidatus Methylobacter favarea
Candidatus Methylobacter oryzae
Candidatus Methylospira mobilis
Candidatus Methylumidiphilus alinenensis
Candidatus Mycobacterium methanotrophicum
Candidatus Mycolicibacterium alkanivorans
Candidatus Nitrospira inopinata
Candidatus Nitrospira nitrificans
Candidatus Nitrospira nitrosa
Candidatus Propionivibrio aalborgensis
Candidatus Rhodoblastus alkanivorans
Candidatus Rokubacteria bacterium
Candidatus Sulfurimonas baltica
Candidatus Sulfurimonas marisnigri
Candidatus Symbiobacter mobilis
Candidatus Terasakiella magnetica
Candidatus Thiodiazotropha endoloripes
Candidatus Thiodiazotropha endolucinida
Caproicibacterium amylolyticum
Carboxydocella
Carboxylicivirga linearis
Carboxylicivirga sp. A043
Catellatospora citrea
Cellulomonas sp. SLBN-39
Cellulosilyticum sp. I15G1012
Cereibacter johrii
Cereibacter sphaeroides 2.4.1
Cereibacter sphaeroides KD131
Cereibacter sphaeroides WS8N
Cereibacter sphaeroides f. sp. denitrificans
Cereibacter sphaeroides
Chitiniphilus shinanonensis
Chitinophaga sp. S165
Chloroflexota bacterium
Chloroherpeton thalassium
Chrysiogenes arsenatis
Citrifermentans bemidjiense
Citrifermentans bremense
Citrifermentans pelophilum
Citrifermentans
Clostridiisalibacter paucivorans
Clostridioides difficile
Clostridium aceticum
Clostridium acetobutylicum
Clostridium algidicarnis
Clostridium algifaecis
Clostridium argentinense
Clostridium autoethanogenum
Clostridium beijerinckii
Clostridium botulinum
Clostridium carboxidivorans
Clostridium carnis
Clostridium cellulovorans
Clostridium chromiireducens
Clostridium formicaceticum
Clostridium gelidum
Clostridium homopropionicum
Clostridium hydrogeniformans
Clostridium indicum
Clostridium intestinale
Clostridium ljungdahlii
Clostridium magnum
Clostridium manihotivorum
Clostridium muellerianum
Clostridium neonatale
Clostridium polynesiense
Clostridium puniceum
Clostridium rectalis
Clostridium saccharobutylicum
Clostridium saccharoperbutylacetonicum
Clostridium sartagoforme
Clostridium sp. AWRP
Clostridium sp. BL-8
Clostridium sp. BNL1100
Clostridium sp. C2-6-12
Clostridium sp. C8
Clostridium sp. DJ247
Clostridium sp. DL-VIII
Clostridium sp. E02
Clostridium sp. HBUAS56010
Clostridium sp. JN-1
Clostridium sp. LS
Clostridium sp. Marseille-P2415
Clostridium sp. Marseille-P299
Clostridium sp. YIM B02506
Clostridium sp. YIM B02555
Clostridium thailandense
Clostridium zeae
Clostridium
Cognatazoarcus halotolerans
Cognaticolwellia aestuarii
Colwellia psychrerythraea
Colwellia sp. MSW7
Comamonadaceae bacterium
Comamonas granuli
Comamonas terrigena
Comamonas testosteroni
Conexibacter sp. DBS9H8
Conexibacter sp. SYSU D00693
Conexibacter sp. W3-3-2
Conexibacter woesei
Crassaminicella profunda
Cryobacterium sp. SO1
Cryptosporangium arvum
Cryptosporangium aurantiacum
Cryptosporangium phraense
Cupidesulfovibrio sp. HK-II
Cupidesulfovibrio sp. SRB-5
Cupidesulfovibrio termitidis
Cupriavidus necator
Cupriavidus pinatubonensis
Curtobacterium citreum
Curtobacterium sp. AG1037
Curvibacter gracilis
Curvibacter lanceolatus
Curvibacter sp. PAE-UM
Curvivirga aplysinae
Cuspidothrix issatschenkoi
Cyanobacterium aponinum
Dechloromonas denitrificans
Dechloromonas hortensis
Dechloromonas sp. A34
Dechloromonas sp. HYN0024
Dechloromonas sp. TW-R-39-2
Deferrisoma camini
Defluviicoccus vanus
Defluviitalea phaphyphila
Defluviitalea raffinosedens
Deltaproteobacteria bacterium
Dendrosporobacter quercicolus
Denitromonas sp. IR12
Denitrovibrio acetiphilus
Derxia gummosa
Derxia lacustris
Desnuesiella massiliensis
Desulfacinum hydrothermale
Desulfacinum infernum
Desulfamplus magnetovallimortis
Desulfatibacillum aliphaticivorans
Desulfatibacillum alkenivorans
Desulfitibacter alkalitolerans
Desulfobacter curvatus
Desulfobacter latus
Desulfobacter postgatei
Desulfobacter vibrioformis
Desulfobacula toluolica
Desulfobaculum xiamenense
Desulfobotulus alkaliphilus
Desulfobotulus mexicanus
Desulfobotulus sp. H1
Desulfobulbus elongatus
Desulfobulbus propionicus
Desulfobulbus rhabdoformis
Desulfocapsa sulfexigens
Desulfocicer vacuolatum
Desulfocurvibacter africanus
Desulfocurvus vexinensis
Desulfofalx alkaliphila
Desulfoglaeba alkanexedens
Desulfogranum japonicum
Desulfogranum mediterraneum
Desulfohalovibrio alkalitolerans
Desulfohalovibrio reitneri
Desulfolithobacter dissulfuricans
Desulfoluna butyratoxydans
Desulfoluna sp. ASN36
Desulfoluna spongiiphila
Desulfolutivibrio sulfoxidireducens
Desulfomicrobium apsheronum
Desulfomicrobium baculatum
Desulfomicrobium escambiense
Desulfomicrobium macestii
Desulfomicrobium norvegicum
Desulfomicrobium sp. ZS1
Desulfomonile tiedjei
Desulfonatronum lacustre
Desulfonatronum thioautotrophicum
Desulfonatronum thiodismutans
Desulfonema ishimotonii
Desulfonema limicola
Desulfonema magnum
Desulfopila aestuarii
Desulfopila sp. IMCC35006
Desulfopila sp. IMCC35008
Desulforapulum autotrophicum
Desulforegula conservatrix
Desulforhopalus singaporensis
Desulforhopalus sp. IMCC35007
Desulfosediminicola flagellatus
Desulfospira joergensenii
Desulfotignum balticum
Desulfotignum phosphitoxidans
Desulfovibrio aminophilus
Desulfovibrio desulfuricans
Desulfovibrio fairfieldensis
Desulfovibrio ferrophilus
Desulfovibrio gilichinskyi
Desulfovibrio inopinatus
Desulfovibrio intestinalis
Desulfovibrio legallii
Desulfovibrio oxyclinae
Desulfovibrio psychrotolerans
Desulfovibrio sp. 3_1_syn3
Desulfovibrio sp. 6_1_46AFAA
Desulfovibrio sp. 86
Desulfovibrio sp. A2
Desulfovibrio sp. DV
Desulfovibrio sp. Fe33
Desulfovibrio sp. JC022
Desulfovibrio sp. TomC
Desulfovibrio sp. UIB00
Desulfovibrio sp. X2
Desulfovibrio subterraneus
Desulfovibrio sulfodismutans
Desulfovibrio vulgaris
Desulfovibrio
Desulfuribacillus alkaliarsenatis
Desulfuribacillus stibiiarsenatis
Desulfurispira natronophila
Desulfurispirillum indicum
Desulfurivibrio alkaliphilus
Desulfuromonas acetexigens
Desulfuromonas acetoxidans
Desulfuromonas soudanensis
Desulfuromonas sp. AOP6
Desulfuromonas sp. CSMB_57
Desulfuromonas sp. DDH964
Desulfuromonas sp. KJ2020
Desulfuromonas sp. TF
Desulfuromonas thiophila
Desulfuromonas versatilis
Desulfuromusa kysingii
Dethiosulfatarculus sandiegensis
Diaphorobacter sp. ED-3
Dissulfurimicrobium hydrothermale
Dissulfurispira thermophila
Dongshaea marina
Dorea amylophila
Dorea longicatena
Dyadobacter jiangsuensis
Edaphobacter modestus
Enemella evansiae
Enterocloster bolteae
Enterovibrio norvegicus
Epulopiscium sp. ‘N.t. morphotype B’
Ethanoligenens harbinense
Eubacteriales
Eubacterium ramulus
Eubacterium sp. 14-2
Euhalothece natronophila
Falcatimonas sp. MSJ-15
Ferribacterium limneticum
Ferriphaselus amnicola
Ferrovum myxofaciens
Fervidicola ferrireducens
Flavonifractor sp. An306
Flexivirga oryzae
Fodinicola acaciae
Formivibrio citricus
Frankia casuarinae
Frankia discariae
Frankia sp. B2
Frankia sp. CcI156
Frankia sp. CgIM4
Frankia sp. CgIS1
Frankia sp. EUN1f
Frankia sp. KB5
Frankia sp. R43
Frankia
Frigoribacterium sp. Leaf44
Fundidesulfovibrio magnetotacticus
Fundidesulfovibrio putealis
Fundidesulfovibrio soli
Fundidesulfovibrio sp. SG106
Fundidesulfovibrio sp. SG127
Fusibacter paucivorans
Gallionella capsiferriformans
Gammaproteobacteria bacterium MOLA455
Gammaproteobacteria bacterium
Geminocystis herdmanii
Geminocystis sp. NIES-3708
Gemmatimonas groenlandica
Geoalkalibacter ferrihydriticus
Geoalkalibacter halelectricus
Geoalkalibacter subterraneus
Geobacter anodireducens
Geobacter argillaceus
Geobacter benzoatilyticus
Geobacter chapellei
Geobacter grbiciae
Geobacter hydrogenophilus
Geobacter luticola
Geobacter metallireducens
Geobacter pickeringii
Geobacter soli
Geobacter sp. AOG1
Geobacter sp. AOG2
Geobacter sp. DSM 9736
Geobacter sp. FeAm09
Geobacter sp. OR-1
Geobacter sp. SVR
Geobacter sulfurreducens
Geobacter
Geofilum rubicundum
Geomesophilobacter sediminis
Geomonas agri
Geomonas anaerohicana
Geomonas azotofigens
Geomonas edaphica
Geomonas ferrireducens
Geomonas limicola
Geomonas nitrogeniifigens
Geomonas oryzae
Geomonas oryzisoli
Geomonas paludis
Geomonas propionica
Geomonas silvestris
Geomonas sp. RF6
Geomonas sp. Red32
Geomonas subterranea
Geomonas terrae
Geomonas
Geopsychrobacter electrodiphilus
Geosporobacter ferrireducens
Geotalea daltonii
Geotalea sp. SG265
Geotalea toluenoxydans
Geotalea uraniireducens
Geothermobacter hydrogeniphilus
Geothrix fermentans
Geothrix sp. SG10
Geothrix sp. SG12
Geothrix sp. SG184
Geothrix sp. SG263
Geovibrio ferrireducens
Geovibrio thiophilus
Gordonia alkanivorans
Gordonia bronchialis
Gordonia humi
Gordonia hydrophobica
Gordonia insulae
Gordonia jinghuaigii
Gordonia polyisoprenivorans
Gordonia rubripertincta
Gordonia sp. ABSL49_1
Gordonia sp. GONU
Gordonia sp. OPL2
Gordonia sp. PDNC005
Gordonia sp. SW 21
Gordonia spumicola
Gordonia tangerina
Gordonia terrae
Gordonia
Gorillibacterium massiliense
Gorillibacterium timonense
Gossypium arboreum
Gottschalkia purinilytica
Gracilinema caldarium
Granulicoccus phenolivorans
Haematobacter missouriensis
Hahella ganghwensis
Halalkalibacterium ligniniphilum
Halanaerobium praevalens
Halanaerobium saccharolyticum
Halanaerobium salsuginis
Halarcobacter anaerophilus
Haliea sp. SAOS-164
Haloactinopolyspora alba
Haloarcula hispanica
Haloarcula sp. JP-Z28
Halobacteriovorax marinus
Halobacteriovorax sp. BALOs_7
Halobacteriovorax sp. DA5
Halobacteroides halobius
Halobellus sp. Atlit-38R
Halochromatium glycolicum
Halochromatium roseum
Halochromatium salexigens
Halodesulfovibrio aestuarii
Halodesulfovibrio sp. MK-HDV
Haloechinothrix alba
Haloechinothrix halophila
Haloglomus sp. ZY41
Haloimpatiens massiliensis
Halomicroarcula salinisoli
Halonatronum saccharophilum
Halopolyspora algeriensis
Halorientalis litorea
Halorientalis marina
Halorientalis pallida
Halorientalis persicus
Halorientalis salina
Halosaccharopolyspora lacisalsi
Halosimplex pelagicum
Halothece sp. PCC 7418
Halothermothrix orenii
Halovenus salina
Hartmannibacter diazotrophicus
Hathewaya massiliensis
Helicobacter apodemus
Helicobacter mesocricetorum
Helicobacter sp. 15-1451
Helicobacter sp. MIT 99-5507
Heliobacterium chlorum
Heliobacterium mobile
Heliomicrobium gestii
Heliomicrobium modesticaldum
Heliomicrobium undosum
Heliorestis acidaminivorans
Heliorestis convoluta
Herbaspirillum sp. ST 5-3
Holophaga foetida
Humibacillus sp. DSM 29435
Humidesulfovibrio mexicanus
Hungatella xylanolytica
Hwanghaeella grinnelliae
Hydrogenibacillus schlegelii
Hydrogenispora ethanolica
Hydrogenophaga aromaticivorans
Hydrogenophaga taeniospiralis
Hyphomicrobium sp.
Ideonella sp. A 288
Idiomarina
Imhoffiella purpurea
Inediibacterium massiliense
Inhella gelatinilytica
Insolitispirillum peregrinum
Intrasporangium oryzae NRRL B-24470
Intrasporangium oryzae
Iocasia fonsfrigidae
Isoptericola halotolerans
Jatrophihabitans endophyticus
Kibdelosporangium aridum
Kibdelosporangium philippinense
Kineococcus rhizosphaerae
Kineococcus xinjiangensis
Kineosporia rhizophila
Kineosporia sp. A_224
Kineosporia sp. R_H_3
Knoellia sp. DB2414S
Kribbella amoyensis
Kribbella capetownensis
Kribbella jiaozuonensis
Kribbella pittospori
Kribbella shirazensis
Kribbella soli
Kribbella sp. VKM Ac-2566
Kribbella sp. VKM Ac-2568
Kribbella sp. VKM Ac-2570
Kribbella sp. VKM Ac-2572
Kribbella speibonae
Kyrpidia spormannii
Kyrpidia tusciae
Labedaea rhizosphaerae
Labilibacter marinus
Labilithrix luteola
Labrys sp. KNU-23
Labrys sp. WJW
Lachnobacterium bovis
Lachnospira eligens
Lachnospiraceae
Lacrimispora aerotolerans
Lacrimispora algidixylanolytica
Lacrimispora amygdalina
Lacrimispora celerecrescens
Lacrimispora sphenoides
Lacrimispora xylanolytica
Lacrimispora
Lactonifactor longoviformis
Lactonifactor
Lamprobacter modestohalophilus
Leadbettera azotonutricia
Leekyejoonella antrihumi
Legionella massiliensis
Leifsonia shinshuensis
Lentzea albidocapillata
Lentzea atacamensis
Lentzea flaviverrucosa
Leptolyngbya sp. BL0902
Leptolyngbya sp. CCY15150
Leptospira bandrabouensis
Leptospira ellinghausenii
Leptospira idonii
Leptospira kobayashii
Leptospira meyeri
Leptospira ognonensis
Leptospira sp. 1 VSF14
Leptospira sp. GIMC2001
Leptospira terpstrae
Leptospira wolbachii
Leptospira
Lichenicoccus roseus
Limisalsivibrio acetivorans
Litorilituus lipolyticus
Litorilituus sediminis
Lucifera butyrica
Lutibacter sp. HS1-25
Magnetococcus marinus
Magnetofaba australis
Magnetospira sp. QH-2
Magnetospirillum aberrantis
Magnetospirillum caucaseum
Magnetospirillum fulvum
Magnetospirillum gryphiswaldense
Magnetospirillum kuznetsovii
Magnetospirillum magneticum
Magnetospirillum magnetotacticum
Magnetospirillum marisnigri
Magnetospirillum molischianum
Magnetospirillum moscoviense
Magnetospirillum sp. 15-1
Magnetospirillum sp. J10
Magnetospirillum sp. LM-5
Magnetospirillum sp. ME-1
Magnetospirillum sp. SS-4
Magnetospirillum sp. UT-4
Magnetospirillum sp. XM-1
Magnetospirillum
Magnetovibrio blakemorei
Mahella australiensis
Maledivibacter halophilus
Malonomonas rubra
Mameliella alba
Mangrovibacterium diazotrophicum
Mangrovibacterium marinum
Maribellus comscasis
Maricaulis alexandrii
Maridesulfovibrio bastinii
Maridesulfovibrio ferrireducens
Maridesulfovibrio frigidus
Maridesulfovibrio hydrothermalis
Maridesulfovibrio salexigens
Maridesulfovibrio zosterae
Marinilabilia rubra
Marinilabilia salmonicolor
Marinobacter dagiaonensis
Marinobacter sp. G11
Marinobacter sp.
Marinobacterium jannaschii
Mariprofundus ferrooxydans
Marvinbryantia formatexigens
Megalodesulfovibrio gigas
Mesorhizobium amorphae CCNWGS0123
Mesorhizobium amorphae
Mesorhizobium atlanticum
Mesorhizobium delmotii
Mesorhizobium hawassense
Mesorhizobium helmanticense
Mesorhizobium kowhaii
Mesorhizobium loti
Mesorhizobium mediterraneum
Mesorhizobium onobrychidis
Mesorhizobium prunaredense
Mesorhizobium sp. 113-1-2
Mesorhizobium sp. 113-3-9
Mesorhizobium sp. B2-1-3A
Mesorhizobium sp. B2-8-9
Mesorhizobium sp. L-2-11
Mesorhizobium sp. L-8-10
Mesorhizobium sp. L-8-3
Mesorhizobium sp. L48C026A00
Mesorhizobium sp. LNHC252B00
Mesorhizobium sp. LSJC280B00
Mesorhizobium sp. M2A.F.Ca.ET.043.02.1.1
Mesorhizobium sp. M2E.F.Ca.ET.154.01.1.1
Mesorhizobium sp. M2E.F.Ca.ET.166.01.1.1
Mesorhizobium sp. M2E.F.Ca.ET.219.01.1.1
Mesorhizobium sp. M4A.F.Ca.ET.020.02.1.1
Mesorhizobium sp. M4A.F.Ca.ET.022.05.2.1
Mesorhizobium sp. M4A.F.Ca.ET.029.04.2.1
Mesorhizobium sp. M4A.F.Ca.ET.050.02.1.1
Mesorhizobium sp. M4A.F.Ca.ET.090.04.2.1
Mesorhizobium sp. M4B.F.Ca.ET.058.02.1.1
Mesorhizobium sp. M5C.F.Ca.ET.164.01.1.1
Mesorhizobium sp. M5C.F.Cr.IN.023.01.1.1
Mesorhizobium sp. M6A.T.Ca.TU.002.02.2.1
Mesorhizobium sp. M6A.T.Ce.TU.002.03.1.1
Mesorhizobium sp. M6A.T.Ce.TU.016.01.1.1
Mesorhizobium sp. M6A.T.Cr.TU.014.01.1.1
Mesorhizobium sp. M6A.T.Cr.TU.016.01.1.1
Mesorhizobium sp. M6A.T.Cr.TU.017.01.1.1
Mesorhizobium sp. M7A.F.Ca.US.010.02.1.1
Mesorhizobium sp. M7A.F.Ca.US.011.01.1.1
Mesorhizobium sp. M7D.F.Ca.US.004.01.2.1
Mesorhizobium sp. M7D.F.Ca.US.004.03.1.1
Mesorhizobium sp. M7D.F.Ca.US.005.01.1.1
Mesorhizobium sp. M9A.F.Ca.ET.002.03.1.2
Mesorhizobium sp. NZP2234
Mesorhizobium sp. NZP2298
Mesorhizobium sp. ORS 3428
Mesorhizobium sp. Root552
Mesorhizobium sp. SARCC-RB16n
Mesorhizobium sp. WSM3860
Mesorhizobium sp. WSM4303
Mesorhizobium sp. WSM4306
Mesorhizobium sp.
Mesorhizobium tamadayense
Mesorhizobium temperatum
Mesorhizobium tianshanense
Mesorhizobium waimense
Mesorhizobium wenxiniae
Methanobacterium formicicum
Methanocaldococcus infernus
Methanolacinia paynteri
Methanolacinia petrolearia
Methanolobus bombayensis
Methanolobus profundi
Methanolobus psychrotolerans
Methanolobus tindarius
Methanolobus vulcani
Methanolobus zinderi
Methanomicrobium sp. W14
Methanonatronarchaeum thermophilum
Methanoregula boonei
Methanosarcina horonobensis
Methanosphaerula palustris
Methanospirillum hungatei
Methanospirillum lacunae
Methanospirillum sp. J.3.6.1-F.2.7.3
Methanospirillum stamsii
Methylacidimicrobium cyclopophantes
Methylacidimicrobium sp. AP8
Methylacidimicrobium sp. B4
Methylacidimicrobium tartarophylax
Methylacidiphilum fumariolicum SolV
Methylacidiphilum fumariolicum
Methylacidiphilum infernorum V4
Methylacidiphilum infernorum
Methylacidiphilum kamchatkense Kam1
Methylacidiphilum kamchatkense
Methylacidiphilum sp. IT5
Methylacidiphilum sp. IT6
Methylacidiphilum sp. Phi
Methylacidiphilum sp. RTK17.1
Methylacidiphilum sp. Yel
Methylibium sp. Root1272
Methylicorpusculum oleiharenae
Methylobacter luteus
Methylobacter marinus
Methylobacter psychrophilus
Methylobacter sp. BlB1
Methylobacter sp. S3L5C
Methylobacter sp. YRD-M1
Methylobacter sp.
Methylobacter tundripaludum SV96
Methylobacter tundripaludum
Methylobacter
Methylobacterium brachiatum
Methylobacterium dankookense
Methylobacterium longum
Methylobacterium oryzae CBMB20
Methylobacterium oryzae
Methylobacterium persicinum
Methylobacterium phyllosphaerae
Methylobacterium radiotolerans
Methylobacterium sp.
Methylocaldum marinum
Methylocaldum szegediense
Methylocapsa acidiphila B2
Methylocapsa acidiphila
Methylocapsa aurea
Methylocapsa palsarum
Methyloceanibacter methanicus
Methyloceanibacter stevinii
Methylocella palustris
Methylocella silvestris BL2
Methylocella silvestris
Methylocella tundrae
Methylococcaceae bacterium AK-K6
Methylococcaceae bacterium BRS-K6
Methylococcaceae bacterium
Methylococcales bacterium
Methylococcus capsulatus str. Bath
Methylococcus capsulatus
Methylococcus geothermalis
Methylococcus sp. 16-5
Methylococcus sp. EFPC2
Methylococcus sp. LS7-MC
Methylococcus sp. Mc7
Methylocucumis oryzae
Methylocystaceae bacterium
Methylocystaceae
Methylocystis bryophila
Methylocystis echinoides
Methylocystis heyeri
Methylocystis hirsuta
Methylocystis parvus OBBP
Methylocystis parvus
Methylocystis rosea SV97
Methylocystis rosea
Methylocystis silviterrae
Methylocystis sp. B8
Methylocystis sp. H15
Methylocystis sp. H4A
Methylocystis sp. H62
Methylocystis sp. H9a
Methylocystis sp. L43
Methylocystis sp. LW5
Methylocystis sp. MJC1
Methylocystis sp. SB2
Methylocystis sp. SC2
Methylocystis sp. SS37A-Re
Methylocystis sp. Sn-Cys
Methylocystis sp. m261
Methylocystis sp.
Methylocystis
Methyloferula stellata AR4
Methyloferula stellata
Methylogaea oryzae JCM 16910
Methylogaea oryzae
Methyloglobulus morosus KoM1
Methyloglobulus morosus
Methylomagnum ishizawai
Methylomarinum vadi
Methylomicrobium lacus
Methylomicrobium sp. RS1
Methylomicrobium sp. wino1
Methylomicrobium
Methylomonas albis
Methylomonas fluvii
Methylomonas koyamae
Methylomonas lenta
Methylomonas methanica
Methylomonas methanica MC09
Methylomonas paludis
Methylomonas rhizoryzae
Methylomonas sp. 11b
Methylomonas sp. DH-1
Methylomonas sp. EFPC1
Methylomonas sp. Kb3
Methylomonas sp. LL1
Methylomonas sp. LW13
Methylomonas sp. LW15
Methylomonas sp. MK1
Methylomonas sp. R-45362
Methylomonas sp. R-45363
Methylomonas sp. R-45364
Methylomonas sp. R-45371
Methylomonas sp. R-45372
Methylomonas sp. R-45374
Methylomonas sp. R-45378
Methylomonas sp. R-45383
Methylomonas sp. R-49799
Methylomonas sp. R-49807
Methylomonas sp. SURF-1
Methylomonas sp. SURF-2
Methylomonas sp. WSC-6
Methylomonas sp. WSC-7
Methylomonas sp. ZR1
Methylomonas sp.
Methylomonas
Methylophaga thiooxydans
Methyloprofundus sedimenti
Methylorubrum populi
Methylosinus sp. 3S-1
Methylosinus sp. B4S
Methylosinus sp. C49
Methylosinus sp. Ce-a6
Methylosinus sp. LW3
Methylosinus sp. LW4
Methylosinus sp. PW1
Methylosinus sp. R-45379
Methylosinus sp. sav-2
Methylosinus sporium
Methylosinus trichosporium OB3b
Methylosinus trichosporium
Methylosinus
Methylosoma difficile
Methylosoma sp. TFB
Methylotenera mobilis
Methyloterricola oryzae
Methylotetracoccus oryzae
Methylotuvimicrobium alcaliphilum
Methylotuvimicrobium buryatense
Methylovirgula sp. HY1
Methylovulum miyakonense HT12
Methylovulum miyakonense
Methylovulum psychrotolerans
Microbacterium hominis
Microbacterium kyungheense
Microbacterium maritypicum
Microbacterium oxydans
Microbacterium sp. SA39
Microbacterium sp. SLBN-146
Microbacterium trichothecenolyticum
Microbacterium
Micrococcus luteus
Microcoleus asticus
Micromonospora sp. A202
Micromonospora sp. M71_S20
Micromonospora sp. MH33
Millisia brevis
Moritella dasanensis
Moritella sp. JT01
Moritella viscosa
Motiliproteus coralliicola
Motiliproteus sediminis
Motiliproteus sp. SC1-56
Mucilaginibacter gracilis
Mucilaginibacter oryzae
Murinocardiopsis flavida
Myceligenerans xiligouense
Mycobacteriaceae
Mycobacterium aquaticum
Mycobacterium avium
Mycobacterium colombiense
Mycobacterium crocinum
Mycobacterium genavense
Mycobacterium goodii
Mycobacterium gordonae
Mycobacterium hodleri
Mycobacterium lentiflavum
Mycobacterium marinum
Mycobacterium paraffinicum
Mycobacterium paragordonae
Mycobacterium paraintracellulare
Mycobacterium saskatchewanense
Mycobacterium sp. AT1
Mycobacterium sp. DL592
Mycobacterium sp. E1747
Mycobacterium sp. E2327
Mycobacterium sp. ITM-2016-00317
Mycobacterium sp. JS623
Mycobacterium sp. MS1601
Mycobacterium sp. Root135
Mycobacterium sp. shizuoka-1
Mycobacterium tuberculosis variant bovis BCG
Mycobacterium tuberculosis
Mycobacterium yunnanensis
Mycobacterium
Mycolicibacter sp. MYC098
Mycolicibacter sp. MYC340
Mycolicibacterium agri
Mycolicibacterium anyangense
Mycolicibacterium aubagnense
Mycolicibacterium austroafricanum
Mycolicibacterium brisbanense
Mycolicibacterium chubuense
Mycolicibacterium cosmeticum
Mycolicibacterium fluoranthenivorans
Mycolicibacterium helvum
Mycolicibacterium hippocampi
Mycolicibacterium iranicum
Mycolicibacterium komossense
Mycolicibacterium litorale
Mycolicibacterium madagascariense
Mycolicibacterium mageritense
Mycolicibacterium mengxianglii
Mycolicibacterium moriokaense
Mycolicibacterium mucogenicum
Mycolicibacterium murale
Mycolicibacterium phlei
Mycolicibacterium rhodesiae
Mycolicibacterium smegmatis
Mycolicibacterium sp. CH28
Mycolicibacterium sp. P9-64
Mycolicibacterium sphagni
Mycolicibacterium stellerae
Mycolicibacterium tokaiense
Mycolicibacterium tusciae
Mycolicibacterium vinylchloridicum
Mycolicibacterium xanthum
Mycolicibacterium
Nakamurella leprariae
Nakamurella panacisegetis
Natranaerovirga hydrolytica
Natranaerovirga pectinivora
Natrarchaeobaculum aegyptiacum
Natronincola peptidivorans
Natronoflexus pectinivorans
Neiella marina
Neisseria gonorrhoeae
Nevskia ramosa
Niallia endozanthoxylica
Niameybacter massiliensis
Nitrincola sp. A-D6
Nitrincola tapanii
Nitrogeniibacter aestuarii
Nitrogeniibacter mangrovi
Nitrosococcus halophilus Nc 4
Nitrosococcus oceani
Nitrosomonas aestuarii
Nitrosomonas marina
Nitrosomonas oligotropha
Nitrospira cf. moscoviensis SBR1015
Niveibacterium microcysteis
Niveibacterium sp. COAC-50
Niveibacterium
Nocardia africana
Nocardia altamirensis
Nocardia colli
Nocardia donostiensis
Nocardia farcinica
Nocardia jiangxiensis
Nocardia miyunensis
Nocardia niigatensis
Nocardia nova
Nocardia sp. DSM 111727
Nocardia
Nocardioides dokdonensis
Nocardioides immobilis
Nocardioides jensenii
Nocardioides luteus
Nocardioides sp. CF8
Nocardioides sp. Soil796
Nocardiopsis sp. Huas11
Nodosilinea nodulosa
Nonomuraea fuscirosea
Nonomuraea sp. PA05
Nonomuraea thailandensis
Noviherbaspirillum aridicola
Noviherbaspirillum sp. UKPF54
Novispirillum itersonii
Novisyntrophococcus fermenticellae
Novosphingobium sp. Fuku2-ISO-50
Novosphingobium sp. PASSN1
Occallatibacter riparius
Oceanidesulfovibrio indonesiensis
Oceanidesulfovibrio marinus
Oharaeibacter diazotrophicus
Oleidesulfovibrio alaskensis
Oleomonas cavernae
Orenia marismortui
Orenia metallireducens
Orrella marina
Oryzomonas japonica
Oryzomonas rubra
Oryzomonas sagensis
Oryzomonas
Oscillatoria sp. FACHB-1407
Oxobacter pfennigii
Paenibacillus borealis
Paenibacillus durus
Paenibacillus forsythiae
Paenibacillus jilunlii
Paenibacillus odorifer
Paenibacillus piscarius
Paenibacillus rhizophilus
Paenibacillus silagei
Paenibacillus sonchi
Paenibacillus sophorae
Paenibacillus sp. FSL H7-0357
Paenibacillus sp. FSL R5-0912
Paenibacillus sp. FSL R7-269
Paenibacillus sp. FSL R7-277
Paenibacillus sp. NFR01
Paenibacillus sp. P46E
Paenibacillus sp. PK3_47
Paenibacillus sp. S150
Paenibacillus sp. YN15
Paenibacillus stellifer
Paenibacillus tengchongensis
Paenibacillus tepidiphilus
Paenibacillus zanthoxyli
Paenibacillus
Paeniclostridium sordellii
Paraburkholderia caribensis
Paraburkholderia hospita
Paraburkholderia madseniana
Paraburkholderia piptadeniae
Paraburkholderia tropica
Paraburkholderia ultramafica
Paraburkholderia unamae
Paraclostridium benzoelyticum
Paraclostridium bifermentans
Paraclostridium dentum
Paracoccaceae bacterium
Paraconexibacter algicola
Paraconexibacter antarcticus
Paramaledivibacter caminithermalis
Pararhodospirillum oryzae
Pararhodospirillum photometricum
Parasulfuritortus cantonensis
Parazoarcus communis
Patulibacter medicamentivorans
Paucibacter sp. KCTC 42545
Paucidesulfovibrio gracilis
Paucidesulfovibrio longus
Pectinatus brassicae
Pectinatus frisingensis
Pelagibaculum spongiae
Pelagicoccus albus
Pelagimonas varians
Pelobacter propionicus
Pelobacter seleniigenes
Pelorhabdus rhamnosifermentans
Pelosinus fermentans
Pelosinus propionicus
Pelosinus sp. Bkl1
Pelosinus sp. UFO1
Pelosinus
Pelovirga terrestris
Peptoclostridium acidaminophilum
Peptoclostridium litorale
Persephonella atlantica
Persephonella hydrogeniphila
Persephonella marina
Persephonella sp. KM09-Lau-8
Petrocella atlantisensis
Petroclostridium xylanilyticum
Phaeobacter sp. CECT 5382
Phormidium sp. FACHB-1136
Phycicoccus sp. HDW14
Pinisolibacter aquiterrae
Piscicoccus intestinalis
Piscirickettsia salmonis
Planktothrix mougeotii
Planktothrix sp. FACHB-1365
Planktothrix
Plantibacter flavus
Plasticicumulans acidivorans
Pleomorphomonas diazotrophica
Pleomorphomonas koreensis
Pleomorphomonas oryzae
Pleomorphomonas sp. JP5
Pleomorphomonas sp. NRK KF1
Pleomorphomonas sp. SM30
Polaromonas sp. 24-62-144
Polaromonas sp. 28-63-22
Polaromonas sp. 35-63-240
Polaromonas sp. 39-63-203
Polynucleobacter cosmopolitanus
Pontiella desulfatans
Pontiella sulfatireligans
Poseidonibacter lekithochrous
Poseidonibacter parvus
Prauserella aidingensis
Prauserella alba
Prauserella cavernicola
Prauserella flavalba
Prauserella sediminis
Prauserella sp. PE36
Prauserella
Prochlorothrix hollandica
Propionispira arboris
Propionispira raffinosivorans
Propionispira
Propionispora hippei
Propionispora sp. 2/2-37
Propionispora vibrioides
Propionivibrio dicarboxylicus
Propionivibrio limicola
Propionivibrio sp. SG131
Prosthecobacter fusiformis
Pseudanabaena biceps
Pseudanabaena sp. ‘Roaring Creek’
Pseudanabaena sp. ABRG5-3
Pseudanabaena sp. BC1403
Pseudanabaena sp. Chao 1811
Pseudanabaena sp. FACHB-1050
Pseudanabaena sp. FACHB-1998
Pseudanabaena sp. FACHB-723
Pseudanabaena sp. SR411
Pseudanabaena sp. UWO310
Pseudanabaena sp. UWO311
Pseudanabaena sp. lw0831
Pseudanabaena yagii
Pseudoalteromonas denitrificans
Pseudoalteromonas sp. C2R02
Pseudobacteriovorax antillogorgiicola
Pseudoclostridium thermosuccinogenes
Pseudodesulfovibrio aespoeensis
Pseudodesulfovibrio alkaliphilus
Pseudodesulfovibrio cashew
Pseudodesulfovibrio hydrargyri
Pseudodesulfovibrio indicus
Pseudodesulfovibrio mercurii
Pseudodesulfovibrio piezophilus
Pseudodesulfovibrio portus
Pseudodesulfovibrio profundus
Pseudodesulfovibrio sediminis
Pseudodesulfovibrio senegalensis
Pseudodesulfovibrio sp. MCM B-1480
Pseudodesulfovibrio sp. zrk46
Pseudodesulfovibrio tunisiensis
Pseudomaricurvus alkylphenolicus
Pseudomonadota bacterium
Pseudomonas aeruginosa
Pseudomonas alcaligenes
Pseudomonas anguilliseptica
Pseudomonas benzenivorans
Pseudomonas fluorescens
Pseudomonas fluvialis
Pseudomonas kurunegalensis
Pseudomonas pharmacofabricae
Pseudomonas resinovorans
Pseudomonas saudiphocaensis
Pseudomonas sp. PDNC002
Pseudomonas sp. TTU2014-080ASC
Pseudonocardia abyssalis
Pseudonocardia acaciae
Pseudonocardia acidicola
Pseudonocardia alaniniphila
Pseudonocardia alni
Pseudonocardia ammonioxydans
Pseudonocardia bannensis
Pseudonocardia broussonetiae
Pseudonocardia cypriaca
Pseudonocardia dioxanivorans
Pseudonocardia endophytica
Pseudonocardia hierapolitana
Pseudonocardia hydrocarbonoxydans
Pseudonocardia kujensis
Pseudonocardia nigra
Pseudonocardia oceani
Pseudonocardia oroxyli
Pseudonocardia parietis
Pseudonocardia petroleophila
Pseudonocardia pini
Pseudonocardia sediminis
Pseudonocardia sp. AL041005-10
Pseudonocardia sp. Ae505_Ps2
Pseudonocardia sp. Ae707_Ps1
Pseudonocardia sp. Ae717_Ps2
Pseudonocardia sp. C8
Pseudonocardia sp. CNS-139
Pseudonocardia sp. DSM 110487
Pseudonocardia sp. EV170527-09
Pseudonocardia sp. H11422
Pseudonocardia sp. H11425
Pseudonocardia sp. HH130629-09
Pseudonocardia sp. HH130630-07
Pseudonocardia sp. ICBG1293
Pseudonocardia sp. KRD291
Pseudonocardia sp. MH-G8
Pseudonocardia sp. SID8383
Pseudonocardia sp. WMMC193
Pseudonocardia spinosispora
Pseudonocardia sulfidoxydans
Pseudonocardia terrae
Pseudonocardia thermophila
Pseudonocardia xinjiangensis
Pseudonocardia
Pseudooceanicola lipolyticus
Pseudothauera hydrothermalis
Pseudothauera lacus
Pseudothauera nasutitermitis
Pseudothauera rhizosphaerae
Pseudovibrio exalbescens
Psychrodesulfovibrio sp. FT415
Psychromonas antarctica
Quatrionicoccus australiensis
Ramlibacter sp. B156
Ramlibacter sp. GTP1
Reichenbachiella sp. ABR2-5
Rhabdochromatium marinum
Rhizobium altiplani
Rhizobium jaguaris
Rhizobium sp.
Rhizomicrobium electricum
Rhizomicrobium palustre
Rhodobacter sp. AKP1
Rhodobium gokarnense
Rhodobium orientis
Rhodoblastus acidophilus
Rhodococcus aetherivorans
Rhodococcus erythropolis
Rhodococcus fascians
Rhodococcus jostii
Rhodococcus koreensis
Rhodococcus kyotonensis
Rhodococcus opacus
Rhodococcus oxybenzonivorans
Rhodococcus pseudokoreensis
Rhodococcus pyridinivorans
Rhodococcus gingshengii
Rhodococcus rhodochrous
Rhodococcus ruber
Rhodococcus sp. 008
Rhodococcus sp. 14C212
Rhodococcus sp. ACPA1
Rhodococcus sp. B50
Rhodococcus sp. CH91
Rhodococcus sp. DK17
Rhodococcus sp. EPR-157
Rhodococcus sp. Eu-32
Rhodococcus sp. FXJ9.536
Rhodococcus sp. JS3073
Rhodococcus sp. KBS0724
Rhodococcus sp. KRD197
Rhodococcus sp. LB1
Rhodococcus sp. M8
Rhodococcus sp. MSC1_016
Rhodococcus sp. NCIMB 12038
Rhodococcus sp. OK302
Rhodococcus sp. USK13
Rhodococcus sp. W8901
Rhodococcus sp. WAY2
Rhodococcus sp. WB1
Rhodococcus sp. WB9
Rhodococcus sp. ZPP
Rhodococcus wratislaviensis
Rhodococcus yunnanensis
Rhodococcus zopfii
Rhodococcus
Rhodocyclaceae bacterium
Rhodocyclus gracilis
Rhodocyclus tenuis
Rhodoferax antarcticus
Rhodoferax fermentans
Rhodoferax ferrireducens
Rhodoferax lacus
Rhodoferax sp. MIZ03
Rhodoferax sp. U11-2br
Rhodomicrobium lacus
Rhodopseudomonas palustris
Rhodopseudomonas rhenobacensis
Rhodopseudomonas sp. AAP120
Rhodopseudomonas sp. BR0M22
Rhodopseudomonas
Rhodospira trueperi
Rhodospirillaceae bacterium
Rhodospirillales bacterium 20-60-12
Rhodospirillum rubrum
Rhodovastum atsumiense
Rhodovulum sulfidophilum
Richelia sinica
Robinsoniella peoriensis
Romboutsia lituseburensis
Roseburia intestinalis
Roseburia inulinivorans
Roseburia
Roseospira goensis
Roseospira marina
Roseospira navarrensis
Roseospira visakhapatnamensis
Roseospirillum parvum
Roseovarius aestuarii
Rubrivivax sp. A210
Rubrobacter aplysinae
Ruminiclostridium cellobioparum
Ruminiclostridium cellulolyticum
Ruminiclostridium hungatei
Ruminiclostridium josui
Ruminiclostridium papyrosolvens
Saccharicrinis fermentans
Saccharopolyspora erythraea NRRL 2338
Saccharopolyspora gloriosae
Saccharopolyspora pogona
Saccharopolyspora shandongensis
Saccharopolyspora sp. ASAGF58
Saccharopolyspora sp. HNM0983
Saccharopolyspora sp. K220
Saccharopolyspora sp. NFXS83
Saccharopolyspora sp. WRP15-2
Saccharothrix carnea
Saccharothrix saharensis
Saccharothrix texasensis
Salegentibacter salegens
Salidesulfovibrio brasiliensis
Salidesulfovibrio onnuriiensis
Salinispira pacifica
Salinivibrio socompensis
Salinivirga cyanobacteriivorans
Scatolibacter rhodanostii
Schinkia azotoformans
Schnuerera ultunensis
Sciscionella marina
Sedimenticola selenatireducens
Sedimenticola sp. SB48
Sedimenticola thiotaurini
Sediminispirochaeta bajacaliforniensis
Sediminispirochaeta smaragdinae
Selenihalanaerobacter shriftii
Seleniivibrio woodruffii
Selenomonas bovis
Selenomonas noxia
Selenomonas sp. oral taxon 478
Selenomonas sputigena ATCC 35185
Selenomonas sputigena
Serpentinicella alkaliphila
Shewanella benthica
Shewanella
Siculibacillus lacustris
Sideroxyarcus emersonii
Sideroxydans lithotrophicus
Silanimonas lenta
Simplicispira psychrophila
Sinanaerobacter chloroacetimidivorans
Siphoviridae sp.
Skermanella aerolata KACC 11604
Skermanella aerolata
Skermanella rosea
Skermanella sp. TT6
Skermanella stibiiresistens SB22
Skermanella stibiiresistens
Solemya velesiana gill symbiont
Solidesulfovibrio aerotolerans
Solidesulfovibrio alcoholivorans
Solidesulfovibrio carbinolicus
Solidesulfovibrio carbinoliphilus
Solidesulfovibrio fructosivorans
Solidesulfovibrio magneticus
Solimonas aquatica
Solirubrobacter ginsenosidimutans
Solirubrobacter pauli
Solirubrobacter phytolaccae
Solirubrobacter sp. URHD0082
Solirubrobacter taibaiensis
Sphaerospermopsis aphanizomenoides
Sphaerospermopsis sp. LEGE 00249
Sphaerospermopsis sp. LEGE 08334
Sphaerotilus hippei
Sphingobium sp. TKS
Sphingobium yanoikuyae
Sphingomonas
Sphingopyxis sp. OPL5
Spirochaeta africana
Spirochaeta cellobiosiphila
Spirochaeta isovalerica
Spirochaeta lutea
Spongiibacter thalassae
Sporobacter termitidis
Sporolituus thermophilus
Sporomusa ovata
Sporomusa silvacetica
Sporomusaceae
Sporosalibacterium faouarense
Staphylococcus aureus
Starkeya novella
Stenotrophomonas maltophilia
Steroidobacter agaridevorans
Steroidobacter cummioxidans
Steroidobacter gossypii
Streptomyces acidiscabies
Streptomyces avidinii
Streptomyces blattellae
Streptomyces brasiliensis
Streptomyces canus
Streptomyces carpinensis
Streptomyces cellulosae
Streptomyces cocklensis
Streptomyces coelicolor A3(2)
Streptomyces dioscori
Streptomyces endocoffeicus
Streptomyces humicola
Streptomyces ipomoeae
Streptomyces ochraceiscleroticus
Streptomyces puniciscabiei
Streptomyces scabiei
Streptomyces sp. 7R016
Streptomyces sp. 840.1
Streptomyces sp. 8L
Streptomyces sp. ADI95-17
Streptomyces sp. Ag109_O5-1
Streptomyces sp. Ag82_O1-15
Streptomyces sp. BK022
Streptomyces sp. BK205
Streptomyces sp. BK208
Streptomyces sp. BK239
Streptomyces sp. BK340
Streptomyces sp. CEV 2-1
Streptomyces sp. DSM 40750
Streptomyces sp. GMR22
Streptomyces sp. HB202
Streptomyces sp. KS 21
Streptomyces sp. KhCrAH-43
Streptomyces sp. NEAU-383
Streptomyces sp. NRRL B-24720
Streptomyces sp. NRRL F-525
Streptomyces sp. PanSC19
Streptomyces sp. RB17
Streptomyces sp. RCU064
Streptomyces sp. Ru73
Streptomyces sp. S4.7
Streptomyces sp. SID14478
Streptomyces sp. SLBN-115
Streptomyces sp. SLBN-134
Streptomyces sp. SLBN-31
Streptomyces sp. T12
Streptomyces sp. TLI_185
Streptomyces sp. TLI_55
Streptomyces sp. TP-A0356
Streptomyces sp. TRM68367
Streptomyces sp. VMFN-G11Ma
Streptomyces sp. WM6386
Streptomyces violens
Streptomyces xylophagus
Stutzerimonas stutzeri
Sulfobacillus acidophilus DSM 10332
Sulfobacillus acidophilus TPY
Sulfobacillus benefaciens
Sulfuricella sp. T08
Sulfurimicrobium lacus
Sulfurimonas lithotrophica
Sulfurimonas marina
Sulfurisoma sediminicola
Sulfuritalea hydrogenivorans
Sulfurivermis fontis
Sulfurospirillum arcachonense
Sulfurospirillum arsenophilum
Sulfurospirillum sp. UCH001
Sunxiuginia elliptica
Synechocystis salina
Syntrophobotulus glycolicus
Syntrophomonas wolfei
Syntrophomonas zehnderi
Syntrophotalea carbinolica
Tardiphaga sp. vice154
Tardiphaga sp. vice278
Tardiphaga sp. vice304
Tardiphaga sp. vice352
Telmatospirillum siberiense
Telmatospirillum sp. J64-1
Tepidanaerobacter acetatoxydans
Tepidimicrobium xylanilyticum
Terasakiella brassicae
Terasakiella pusilla
Terasakiella sp. SH-1
Teredinibacter haidensis
Thalassomonas actiniarum
Thalassomonas haliotis
Thalassomonas viridans
Thalassotalea algicola
Thauera aminoaromatica
Thauera butanivorans
Thauera linaloolentis
Thauera propionica
Thauera sp. CAU 1555
Thauera sp. SWB20
Thauera sp. Sel9
Thaumarchaeota archae on
Thermasporomyces composti
Thermincola ferriacetica
Thermincola
Thermoanaerobacter kivui
Thermoanaerobacter sp. A7A
Thermoanaerobacter sp. YS13
Thermoanaerobacter
Thermoanaerobacterium butyriciformans
Thermoanaerobacterium saccharolyticum
Thermoanaerobacterium sp. PSU-2
Thermoanaerobacterium sp. RBIITD
Thermoanaerobacterium thermosaccharolyticum
Thermoanaerobacterium xylanolyticum
Thermoanaerobacterium
Thermobrachium celere
Thermoclostridium caenicola
Thermoclostridium stercorarium
Thermodesulfatator atlanticus
Thermodesulfatator autotrophicus
Thermodesulfatator indicus
Thermodesulfobacterium commune
Thermodesulfobacterium geofontis
Thermodesulfobacterium thermophilum
Thermodesulfobium acidiphilum
Thermodesulfobium narugense
Thermodesulforhabdus norvegica
Thermogemmatispora tikiterensis
Thermohalobacter berrensis
Thermomicrobium roseum
Thermosediminibacter oceani
Thermospira aquatica
Thermosulfuriphilus ammonigenes
Thermotalea metallivorans
Thioalbus denitrificans
Thioalkalivibrio sp. ALJ15
Thioalkalivibrio sp. ALJ2
Thioalkalivibrio sp. ALJ3
Thiobaca trueperi
Thiocapsa bogorovii
Thiocapsa imhoffii
Thiocapsa marina
Thiocapsa rosea
Thiocapsa roseopersicina
Thiocapsa sp. KS1
Thiocystis violacea
Thioflexithrix psekupsensis
Thiohalocapsa halophila
Thiohalocapsa sp. ML1
Thiohalocapsa sp. PB-PSB1
Thiolapillus brandeum
Thiomonas sp. FB-Cd
Thiorhodococcus drewsii
Thiorhodococcus mannitoliphagus
Thiorhodococcus minor
Thiospirillum jenense
Thiospirochaeta perfilievii
Tissierella praeacuta
Tissierella simiarum
Tolumonas auensis
Tolumonas lignilytica
Tolumonas osonensis
Trebonia kvetii
Treponema berlinense
Treponema brennaborense
Treponema denticola
Treponema parvum
Treponema porcinum
Treponema primitia
Treponema sp. J25
Treponema sp. JC4
Treponema sp. Marseille-Q4132
Treponema succinifaciens
Treponema vincentii
Treponema zuelzerae
Treponema
Trichlorobacter lovleyi
Trichlorobacter thiogenes
Turneriella parva
Uliginosibacterium aquaticum
Uliginosibacterium gangwonense
Uliginosibacterium sp. TH139
Uliginosibacterium
Undibacterium flavidum
Undibacterium umbellatum
Undibacterium
Vallitalea guaymasensis
Vallitalea pronyensis
Variimorphobacter saccharofermentans
Variovorax sp. E3
Variovorax sp. PBL-H6
Varunaivibrio sulfuroxidans
Verminephrobacter eiseniae EF01-2
Verrucomicrobium sp. 3C
Vibrio aerogenes
Vibrio albus
Vibrio campbellii
Vibrio cholerae
Vibrio fortis
Vibrio hyugaensis
Vibrio mangrovi
Vibrio mediterranei
Vibrio ponticus
Vibrio quintilis
Vibrio rhizosphaerae
Vibrio sp. MEBiC08052
Vibrio sp. kj40-1
Vibrio tubiashii
Vibrio vulnificus
Vibrio
Virgisporangium aurantiacum
Vitreoscilla filiformis
Vulcanibacillus modesticaldus
Williamsia limnetica
Williamsia soli
Williamsia sp. 1138
Xanthobacter dioxanivorans
Xanthomonas citri
Xinfangfangia humi
Xylophilus rhododendri
Xylophilus sp. ASV27
Yoonia vestfoldensis
Zavarzinia aquatilis
Zoogloea dura
Zoogloea oryzae
Zoogloea sp. 1C4
Zoogloea sp. LCSB751
Eubacterium tenue
Halarcobacter arenosus
Phormidium sp. ETS-05
Ruminococcus torques
bacterium HR30
bacterium M00.F.Ca.ET.159.01.1.1
bacterium
candidate division Zixibacteria bacterium
endosymbiont of Lamellibrachia barhami
endosymbiont of Ridgeia piscesae
marine metagenome
methanotrophic bacterial endosymbiont of Bathymodiolus sp.
mine drainage metagenome
sulfur-oxidizing endosymbiont of Gigantopelta aegis
unclassified Amycolatopsis
unclassified Anaeromyxobacter
unclassified Anaerotruncus
unclassified Azospirillum
unclassified Bradyrhizobium
unclassified Burkholderia
unclassified Caballeronia
unclassified Clostridium
unclassified Colwellia
unclassified Desulfovibrio
unclassified Fusibacter
unclassified Geothrix
unclassified Haloarcula
unclassified Halomicroarcula
unclassified Kribbella
unclassified Leptolyngbya
unclassified Limnothrix
unclassified Methylocaldum
unclassified Methylocystis
unclassified Methylomonas
unclassified Methylosinus
unclassified Mycobacterium
unclassified Mycolicibacter
unclassified Nocardioides
unclassified Poseidonibacter
unclassified Pseudodesulfovibrio
unclassified Pseudonocardia
unclassified Rhodococcus
unclassified Simplicispira
unclassified Streptomyces
unclassified Synechocystis
unclassified Treponema
uncultured Alphaproteobacteria bacterium
uncultured Gammaproteobacteria bacterium
uncultured Methylobacter sp.
uncultured Methylocella sp.
uncultured Methylocystis sp.
uncultured Methylosinus sp.
uncultured ammonia-oxidizing bacterium
uncultured archaeon
uncultured bacterium
uncultured beta proteobacterium
uncultured methanotrophic bacterium
uncultured microorganism
uncultured prokaryote
uncultured proteobacterium
uncultured soil bacterium

In some embodiments, the species present in the consortium of bacteria may be determined by sequencing methods. In some embodiments, targeted sequencing may be used to determine the species present in the bacterial consortium. In some embodiments, metagenomic sequencing may be used to determine the species present in the bacterial consortium. In certain embodiments, 16S sequencing may be used to determine the species present in the bacterial consortium. In various embodiments, shotgun metagenomic sequencing methods may be used to determine the species present in the bacterial consortium. In some embodiments, next generation sequencing may be used. In certain embodiments, nanopore sequencing may be used. In certain embodiments, Oxford Nanopore sequencing may be used. In certain embodiments, long read sequencing may be used. In certain embodiments, short read sequencing may be used. In certain embodiments, Illumina sequencing may be used. In certain embodiments, PacBio sequencing may be used. In various cases, Ultima sequencing may be used. In some cases, Elements sequencing may be used. In some embodiments, a ZymoBIOMICS® Shotgun Metagenomic Sequencing method may be used.

In certain embodiments, a sequencing method may comprise a DNA isolation step. In some embodiments, a DNA isolation step may comprise a ZymoBIOMICS®-96 MagBead DNA Kit (Zymo Research, Irvine, CA) using an automated platform. In some embodiments, ZymoBIOMICS® DNA Miniprep Kit (Zymo Research, Irvine, CA) may be used. In some embodiments, for example for samples with low biomass, a ZymoBIOMICS® DNA Microprep Kit (Zymo Research, Irvine, CA) may be used. In certain embodiments, DNA extraction methods known in the art may be used. In some embodiments, DNA extraction methods may be column based or may use organic solvents. In certain embodiments, DNA extraction methods enable sensitive detection of low abundance bacteria or archaea. In certain embodiments, DNA extraction methods enable sensitive detection of high and low abundance bacteria or archaea.

In some embodiments, the bacterial consortium disclosed herein is capable of consuming methane (CH₄) at a rate greater than 13.5 umol/gDW per day and fixing nitrogen (N₂) at a rate greater than 2.0 umol/gDW per day. In certain embodiments, the bacterial consortium disclosed herein is capable of consuming methane (CH₄) at a rate greater than 5 umol/gDW per day and fixing nitrogen (N₂) at a rate greater than 2.5 umol/gDW per day. In certain embodiments, the bacterial consortium consumes methane (CH₄) at a rate greater than 5 umol/gDW per day. In certain embodiments, the consortium of bacteria consumes methane (CH₄) at a rate greater than 13.5 umol/gDW per day. In certain embodiments, the bacterial consortium fixes nitrogen (N₂) at a rate greater than 2.0 umol/gDW per day. In certain embodiments, the bacterial consortium fixes nitrogen (N₂) at a rate greater than 2.5 umol/gDW per day. In certain embodiments, a first bacterium in the consortium of bacteria is unaffected by a second bacterium in the consortium of bacteria in respect to the first bacterium's ability to fix nitrogen. In various embodiments, a first bacterium in the consortium of bacteria is unaffected by a second bacterium in the consortium of bacteria in respect to the first bacterium's ability to consume CH₄.

### III. Viability of Consortium

Disclosed herein is a bacterial consortium capable of consuming methane and fixing nitrogen. In some embodiments, the bacterial consortium capable of consuming methane and fixing nitrogen may be viable for days, weeks, or months. In some embodiments, the viability of the consortium is defined by the percent survival of bacteria in the consortium of bacteria. In some embodiments, the viability of the consortium of bacteria may comprise about 10% to about 99% survival for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer.

In certain embodiments, the viability of the consortium of bacteria may comprise about 10% to about 20%, about 10% to about 30%, about 10% to about 40%, about 10% to about 50%, about 10% to about 60%, about 10% to about 70%, about 10% to about 80%, about 10% to about 85%, about 10% to about 90%, about 10% to about 95%, about 10% to about 99%, about 20% to about 30%, about 20% to about 40%, about 20% to about 50%, about 20% to about 60%, about 20% to about 70%, about 20% to about 80%, about 20% to about 85%, about 20% to about 90%, about 20% to about 95%, about 20% to about 99%, about 30% to about 40%, about 30% to about 50%, about 30% to about 60%, about 30% to about 70%, about 30% to about 80%, about 30% to about 85%, about 30% to about 90%, about 30% to about 95%, about 30% to about 99%, about 40% to about 50%, about 40% to about 60%, about 40% to about 70%, about 40% to about 80%, about 40% to about 85%, about 40% to about 90%, about 40% to about 95%, about 40% to about 99%, about 50% to about 60%, about 50% to about 70%, about 50% to about 80%, about 50% to about 85%, about 50% to about 90%, about 50% to about 95%, about 50% to about 99%, about 60% to about 70%, about 60% to about 80%, about 60% to about 85%, about 60% to about 90%, about 60% to about 95%, about 60% to about 99%, about 70% to about 80%, about 70% to about 85%, about 70% to about 90%, about 70% to about 95%, about 70% to about 99%, about 80% to about 85%, about 80% to about 90%, about 80% to about 95%, about 80% to about 99%, about 85% to about 90%, about 85% to about 95%, about 85% to about 99%, about 90% to about 95%, about 90% to about 99%, or about 95% to about 99% survival for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer.

In certain embodiments, the viability of the consortium of bacteria may comprise about 10%, about 20%, about 30%, about 40%, about 50%, about 60%, about 70%, about 80%, about 85%, about 90%, about 95%, or about 99% survival for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. In certain embodiments, the viability of the consortium of bacteria may comprise at least about 10%, about 20%, about 30%, about 40%, about 50%, about 60%, about 70%, about 80%, about 85%, about 90%, or about 95% survival for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. In certain embodiments, the viability of the consortium of bacteria may comprise at most about 20%, about 30%, about 40%, about 50%, about 60%, about 70%, about 80%, about 85%, about 90%, about 95%, about 99%, or about 100% survival for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer.

In some embodiments, the viability of the consortium of bacteria may comprise about 10% to about 99% survival for at least about 2 months, about 3 months, about 4 months, about 5 months, about 6 months, about 7 months, about 8 months, about 9 months, about 10 months, about 11 months, about 12 weeks, or longer.

In certain embodiments, the viability of the consortium of bacteria may comprise about 10% to about 20%, about 10% to about 30%, about 10% to about 40%, about 10% to about 50%, about 10% to about 60%, about 10% to about 70%, about 10% to about 80%, about 10% to about 85%, about 10% to about 90%, about 10% to about 95%, about 10% to about 99%, about 20% to about 30%, about 20% to about 40%, about 20% to about 50%, about 20% to about 60%, about 20% to about 70%, about 20% to about 80%, about 20% to about 85%, about 20% to about 90%, about 20% to about 95%, about 20% to about 99%, about 30% to about 40%, about 30% to about 50%, about 30% to about 60%, about 30% to about 70%, about 30% to about 80%, about 30% to about 85%, about 30% to about 90%, about 30% to about 95%, about 30% to about 99%, about 40% to about 50%, about 40% to about 60%, about 40% to about 70%, about 40% to about 80%, about 40% to about 85%, about 40% to about 90%, about 40% to about 95%, about 40% to about 99%, about 50% to about 60%, about 50% to about 70%, about 50% to about 80%, about 50% to about 85%, about 50% to about 90%, about 50% to about 95%, about 50% to about 99%, about 60% to about 70%, about 60% to about 80%, about 60% to about 85%, about 60% to about 90%, about 60% to about 95%, about 60% to about 99%, about 70% to about 80%, about 70% to about 85%, about 70% to about 90%, about 70% to about 95%, about 70% to about 99%, about 80% to about 85%, about 80% to about 90%, about 80% to about 95%, about 80% to about 99%, about 85% to about 90%, about 85% to about 95%, about 85% to about 99%, about 90% to about 95%, about 90% to about 99%, or about 95% to about 99% survival for at least about 2 months, about 3 months, about 4 months, about 5 months, about 6 months, about 7 months, about 8 months, about 9 months, about 10 months, about 11 months, about 12 weeks, or longer.

In certain embodiments, the viability of the consortium of bacteria may comprise about 10%, about 20%, about 30%, about 40%, about 50%, about 60%, about 70%, about 80%, about 85%, about 90%, about 95%, or about 99% survival for at least about 2 months, about 3 months, about 4 months, about 5 months, about 6 months, about 7 months, about 8 months, about 9 months, about 10 months, about 11 months, or about 12 months. In certain embodiments, the viability of the consortium of bacteria may comprise at least about 10%, about 20%, about 30%, about 40%, about 50%, about 60%, about 70%, about 80%, about 85%, about 90%, or about 95% survival for at least about 2 months, about 3 months, about 4 months, about 5 months, about 6 months, about 7 months, about 8 months, about 9 months, about 10 months, about 11 months, or about 12 months. In certain embodiments, the viability of the consortium of bacteria may comprise at most about 20%, about 30%, about 40%, about 50%, about 60%, about 70%, about 80%, about 85%, about 90%, about 95%, about 99%, or about 100% survival for at least about 2 months, about 3 months, about 4 months, about 5 months, about 6 months, about 7 months, about 8 months, about 9 months, about 10 months, about 11 months, or about 12 months.

In certain embodiments, the viability may be determined by the features of the consortium collectively. In some embodiments, the viability of the consortium may be determined by the amount of methane consumed. In some embodiments, the bacterial consortium consumes methane at a rate greater than 5 umol/gDW per day. In various embodiments, the consortium of bacteria consumes methane at a rate greater than 13.5 umol/gDW per day. In certain embodiments, the viability of the consortium may be determined by the amount of nitrogen that is fixed. In some embodiments, the consortium of bacteria fixes nitrogen at a rate greater than 2.0 umol/gDW per day. In various embodiments, the consortium of bacteria fixes nitrogen at a rate greater than 2.5 umol/gDW per day. In certain embodiments, the consortium of bacteria is unaffected by a second bacterium in the consortium of bacteria in respect to the first bacterium's ability to fix nitrogen. In some embodiments, the consortium of bacteria is unaffected by a second bacterium in the consortium of bacteria in respect to the first bacterium's ability to consume CH₄.

The consortium of bacteria as disclosed herein may comprise at least 1, 2, 3, 4, or more different bacterial phyla. In certain non-limiting embodiments, the consortium of bacteria may comprise Micrarchaeota, Actinobacteria, Bacteroidetes, Firmicutes, or Proteobacteria. In certain embodiments, the consortium of bacteria comprises at least 1, 2, 3, 4, 5, 6, 7, 8, 9, 10, 15, 20, 30, or more different bacterial order. In some cases, the consortium of bacteria comprises bacteria classified by order as belonging to, for example, Rhizobiales, Methylococcales, Methanobacteriales, Methanosarcinales, Nitrosomonadales, or Pseudomonadale. In some embodiments, the consortium of bacteria may comprise bacteria from at least 1, 2, 3, 4, 5, 6, 7, 8, 9, 10, 15, 20, 30, 40, 50, or more different bacterial family. In certain embodiments, non-limiting examples of the families to which bacteria disclosed herein belong may comprise Methylocystaceae, Methylococcaceae, Methanobacteriaceae, Methanosarcinaceae, Methylophilacea, or Pseudonocardiaceae. In some embodiments, the consortium of bacteria may comprise bacteria from at least 1, 2, 3, 4, 5, 6, 7, 8, 9, 10, 15, 20, 30, 40, 50, or more different bacterial genera. In certain embodiments, the consortium of bacteria may comprise Azospirillum, Amycolatopsis, Acidiphilium, Burkholderia, Methylosinus, Methylocystis, Methylopila, Mesorhizobium Rhodococcus, Pseudonocardia, Pseudomonas, Streptomyces, Nocardia, Mycolicibacterium, or Mycobacterium. In certain embodiments, the consortium of bacteria may comprise bacteria from at least 1, 2, 3, 4, 5, 6, 7, 8, 9, 10, 15, 20, 30, 40, 50, or more different species. In some embodiments, the consortium of bacteria may comprise bacteria from different species that belong to the same genus, family, order, or phylum.

In some embodiments, the consortium of bacteria may comprise 1, 2, 3, 4, 5, 6, 7, 8, 9, 10, or more species from Tables 1, 3, or 4. In certain embodiments, the consortium of bacteria may comprise one or more species from Tables 1, 3, or 4 in combination with other bacteria that may consume methane only, fix nitrogen only, or do neither consume methane nor fix nitrogen. In various embodiments, the consortium of bacteria may comprise only bacteria listed in Tables 1, 3, or 4.

In some embodiments, the viability of the bacterial consortium may be determined by measuring the methane consumption or nitrogen fixation of the consortium of bacteria. In some embodiments, the viability of the bacterial consortium may be determined by measuring the methane consumption and nitrogen fixation of the consortium of bacteria.

In some embodiments, the viability of the consortium may be determined in ambient outdoor conditions. In some embodiments, the viability of the consortium may be determined in field conditions. In certain embodiments, the viability of the consortium may be determined in a compost reactor or bioreactor. In some embodiments, the viability of the consortium may be determined in greenhouse conditions. In some embodiments, the viability of the consortium may be determined in laboratory conditions. In some embodiments, the viability of the consortium is measured at sea level. In some embodiments, the viability of the consortium is measured at elevation. In certain embodiments, the viability of the consortium improves at higher elevations. In certain embodiments, the viability of the consortium improves at lower elevations.

In various embodiments, the bacterial consortium is viable after freeze-drying, spray-freeze, lyophilization, or refrigeration. In some embodiments, the methane consumption and nitrogen fixation capability of the bacterial consortium is maintained following freeze-dry, spray-freeze, lyophilization, or refrigeration.

In certain embodiments, the bacterial consortium is viable at room temperature for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. In certain embodiments, the bacterial consortium is viable above room temperature for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. In certain embodiments, the bacterial consortium is viable below room temperature for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. In some embodiments, the bacterial consortium is viable at 0° C., 2° C., 4° C., 6° C., 8° C., 10° C., 15° C., 20° C., 25° C., 30° C., 35° C., 40° C., 45° C., or at higher temperatures for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. In some embodiments, the bacterial consortium can survive at −20° C., −15° C., −10° C., −5° C., or 0° C. for at least about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer.

In certain embodiments, the bacterial consortium is viable at room temperature for at least about for at least about 2 months, about 3 months, about 4 months, about 5 months, about 6 months, about 7 months, about 8 months, about 9 months, about 10 months, about 11 months, about 12 months, or longer. In certain embodiments, the bacterial consortium is viable above room temperature for at least about 1 month, about 2 months, about 3 months, about 4 months, about 5 months, about 6 months, about 7 months, about 8 months, about 9 months, about 10 months, about 11 months, about 12 months, or longer. In certain embodiments, the bacterial consortium is viable below room temperature for at least 1 month, about 2 months, about 3 months, about 4 months, about 5 months, about 6 months, about 7 months, about 8 months, about 9 months, about 10 months, about 11 months, about 12 months, or longer. In some embodiments, the bacterial consortium is viable at 0° C., 2° C., 4° C., 6° C., 8° C., 10° C., 15° C., 20° C., 25° C., 30° C., 35° C., 40° C., 45° C., or at higher temperatures for at least about for at least about 1 month, about 2 months, about 3 months, about 4 months, about 5 months, about 6 months, about 7 months, about 8 months, about 9 months, about 10 months, about 11 months, about 12 months, or longer. In some embodiments, the bacterial consortium can survive at −20° C., −15° C., −10° C., −5° C., or 0° C. for at least about for at least about 1 month, about 2 months, about 3 months, about 4 months, about 5 months, about 6 months, about 7 months, about 8 months, about 9 months, about 10 months, about 11 months, about 12 months, or longer.

In certain embodiments, the rate of nitrogen fixation and methane consumption by the consortium of bacteria decreases at low temperatures. In some cases, the rate of nitrogen fixation and methane consumption by the consortium of bacteria can improve when the consortium or bacteria is heated. In some instances, the methane consumption and nitrogen fixing capability of the bacterial consortium decreases as temperature decreases. In some embodiments, the methane consumption and nitrogen fixing capability of the bacterial consortium can increased as temperature increases. In some cases, the methane consumption and nitrogen fixing capability of the consortium of bacteria decreases at temperatures higher than 40° C., 45° C., 50° C., or higher. In some instances, the bacterial consortium may be dormant such that the methane consumption and nitrogen fixation is effectively 0 gmol/gDW. In some cases, the bacterial consortium may reactivate or leave dormancy such that the consortium may resume consumption of methane and nitrogen fixation.

In certain embodiments, the bacterial consortium is capable of consuming methane and fixing nitrogen for about 1 week to about 6 weeks. In certain embodiments, the bacterial consortium is capable of consuming methane and fixing nitrogen for about 1 week to about 2 weeks, about 1 week to about 3 weeks, about 1 week to about 4 weeks, about 1 week to about 6 weeks, about 2 weeks to about 3 weeks, about 2 weeks to about 4 weeks, about 2 weeks to about 6 weeks, about 3 weeks to about 4 weeks, about 3 weeks to about 6 weeks, or about 4 weeks to about 6 weeks. In certain embodiments, the bacterial consortium is capable of consuming methane and fixing nitrogen for about 1 week, about 2 weeks, about 3 weeks, about 4 weeks, or about 6 weeks. In certain embodiments, the bacterial consortium is capable of consuming methane and fixing nitrogen for at least about 1 week, about 2 weeks, about 3 weeks, or about 4 weeks. In certain embodiments, the bacterial consortium is capable of consuming methane and fixing nitrogen for at most about 2 weeks, about 3 weeks, about 4 weeks, or about 6 weeks.

In certain embodiments, the bacterial consortium is capable of consuming methane and fixing nitrogen for about 1 month to about 12 months. In certain embodiments, the bacterial consortium is capable of consuming methane and fixing nitrogen for about 1 month to about 2 months, about 1 month to about 3 months, about 1 month to about 4 months, about 1 month to about 5 months, about 1 month to about 6 months, about 1 month to about 7 months, about 1 month to about 8 months, about 1 month to about 9 months, about 1 month to about 10 months, about 1 month to about 11 months, about 1 month to about 12 months, about 2 months to about 3 months, about 2 months to about 4 months, about 2 months to about 5 months, about 2 months to about 6 months, about 2 months to about 7 months, about 2 months to about 8 months, about 2 months to about 9 months, about 2 months to about 10 months, about 2 months to about 11 months, about 2 months to about 12 months, about 3 months to about 4 months, about 3 months to about 5 months, about 3 months to about 6 months, about 3 months to about 7 months, about 3 months to about 8 months, about 3 months to about 9 months, about 3 months to about 10 months, about 3 months to about 11 months, about 3 months to about 12 months, about 4 months to about 5 months, about 4 months to about 6 months, about 4 months to about 7 months, about 4 months to about 8 months, about 4 months to about 9 months, about 4 months to about 10 months, about 4 months to about 11 months, about 4 months to about 12 months, about 5 months to about 6 months, about 5 months to about 7 months, about 5 months to about 8 months, about 5 months to about 9 months, about 5 months to about 10 months, about 5 months to about 11 months, about 5 months to about 12 months, about 6 months to about 7 months, about 6 months to about 8 months, about 6 months to about 9 months, about 6 months to about 10 months, about 6 months to about 11 months, about 6 months to about 12 months, about 7 months to about 8 months, about 7 months to about 9 months, about 7 months to about 10 months, about 7 months to about 11 months, about 7 months to about 12 months, about 8 months to about 9 months, about 8 months to about 10 months, about 8 months to about 11 months, about 8 months to about 12 months, about 9 months to about 10 months, about 9 months to about 11 months, about 9 months to about 12 months, about 10 months to about 11 months, about 10 months to about 12 months, or about 11 months to about 12 months. In certain embodiments, the bacterial consortium is capable of consuming methane and fixing nitrogen for about 1 month, about 2 months, about 3 months, about 4 months, about 5 months, about 6 months, about 7 months, about 8 months, about 9 months, about 10 months, about 11 months, or about 12 months. In certain embodiments, the bacterial consortium is capable of consuming methane and fixing nitrogen for at least about 1 month, about 2 months, about 3 months, about 4 months, about 5 months, about 6 months, about 7 months, about 8 months, about 9 months, about 10 months, or about 11 months. In certain embodiments, the bacterial consortium is capable of consuming methane and fixing nitrogen for at most about 2 months, about 3 months, about 4 months, about 5 months, about 6 months, about 7 months, about 8 months, about 9 months, about 10 months, about 11 months, or about 12 months.

In some embodiments, the viability of the consortium may be improved with addition of additives. In some embodiments, the additives may improve the overall health and metabolic activity of one or more bacteria. In certain embodiments, the additives may improve the methane consuming capacity of a bacteria. In some embodiments, the additives may improve the nitrogen fixation capacity of a bacteria. In various embodiments, the additives may improve the ability of the consortium to recover from spray drying, freeze drying, or lyophilization. In certain embodiments, the additives may improve the ability of the consortium to recover from low temperatures or low oxygen environments, such as at high altitudes. In some embodiments, the additives may improve the ability of the consortium of bacteria to outcompete other bacteria.

### IV. Additives

In some embodiments, a consortium of bacteria described herein comprises one or more additives. In some embodiments, the additives improve overall health of one or more bacteria in the consortium of bacteria. In certain embodiments, the additives may improve the methane consuming capacity of one or more bacteria in the consortium or of the collective bacterial consortium. In some embodiments, the additives may improve the nitrogen fixation capacity of one or more bacteria in the consortium or of the collective bacterial consortium. In some embodiments, the additives improve the methane consumption and nitrogen fixation capability of one or more bacteria in the consortium or of the collective bacterial consortium.

In some embodiments, the additives may be added to a consortium of bacteria that may comprise 1, 2, 3, 4, 5, 6, 7, 8, 9, 10, or more species from Tables 1, 3, or 4. In certain embodiments, the additives may be added to a consortium of bacteria that may comprise one or more species from Tables 1, 3, or 4 in combination with other bacteria that may consume methane only, fix nitrogen only, or do neither consume methane nor fix nitrogen. In various embodiments, the additives may be added to a consortium of bacteria that may comprise only bacteria listed in Tables 1, 3, or 4.

In various embodiments, the additives may improve the ability of the consortium to recover from spray drying, freeze drying, or lyophilization. In certain embodiments, the additives may improve the ability of the consortium to recover from low temperatures or low oxygen environments. In certain embodiments, the additives may be used to enrich for a particular bacteria or bacterial feature. In various embodiments, one or more additives may be applied to the consortium of bacteria to aid in the viability of the consortium of bacteria during or after stress. Examples of stress may include, but are not limited to, freeze-dry, spray-freeze, lyophilization, UV exposure, hypoxia, hyperoxia, low nitrogen conditions, excessive heat, physical or mechanical disturbance, acidic pH, neutral pH, basic pH, low moisture conditions, excessive moisture conditions, flooding, drought, freezing, thawing, any combination thereof, or any other chemical, biological, or physical disruption. In some embodiments, the additives may improve the ability of the consortium of bacteria to outcompete other bacteria.

In certain embodiments, the additives may improve the survival of the consortium of bacteria as compared to the consortium of bacteria without the additives. In some embodiments, the additives may improve the survival of the consortium of bacteria as compared to the consortium of bacteria without the additives by about 10% to about 500%. In some embodiments, the additives may improve the survival of the consortium of bacteria as compared to the consortium of bacteria without the additives by about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 10% to about 200%, about 10% to about 500%, about 20% to about 30%, about 20% to about 40%, about 20% to about 50%, about 20% to about 60%, about 20% to about 70%, about 20% to about 80%, about 20% to about 90%, about 20% to about 100%, about 20% to about 200%, about 20% to about 500%, about 30% to about 40%, about 30% to about 50%, about 30% to about 60%, about 30% to about 70%, about 30% to about 80%, about 30% to about 90%, about 30% to about 100%, about 30% to about 200%, about 30% to about 500%, about 40% to about 50%, about 40% to about 60%, about 40% to about 70%, about 40% to about 80%, about 40% to about 90%, about 40% to about 100%, about 40% to about 200%, about 40% to about 500%, about 50% to about 60%, about 50% to about 70%, about 50% to about 80%, about 50% to about 90%, about 50% to about 100%, about 50% to about 200%, about 50% to about 500%, about 60% to about 70%, about 60% to about 80%, about 60% to about 90%, about 60% to about 100%, about 60% to about 200%, about 60% to about 500%, about 70% to about 80%, about 70% to about 90%, about 70% to about 100%, about 70% to about 200%, about 70% to about 500%, about 80% to about 90%, about 80% to about 100%, about 80% to about 200%, about 80% to about 500%, about 90% to about 100%, about 90% to about 200%, about 90% to about 500%, about 100% to about 200%, about 100% to about 500%, or about 200% to about 500%. In some embodiments, the additives may improve the survival of the consortium of bacteria as compared to the consortium of bacteria without the additives by about 10%, about 20%, about 30%, about 40%, about 50%, about 60%, about 70%, about 80%, about 90%, about 100%, about 200%, or about 500%. In some embodiments, the additives may improve the survival of the consortium of bacteria as compared to the consortium of bacteria without the additives by at least about 10%, about 20%, about 30%, about 40%, about 50%, about 60%, about 70%, about 80%, about 90%, about 100%, or about 200%. In some embodiments, the additives may improve the survival of the consortium of bacteria as compared to the consortium of bacteria without the additives by at most about 20%, about 30%, about 40%, about 50%, about 60%, about 70%, about 80%, about 90%, about 100%, about 200%, or about 500%.

In certain embodiments, one or more additives are in an amount sufficient to favor consumption of the CH₄ by the consortium over consumption of another organic molecule. In certain embodiments, one or more additives are in a sufficient amount to keep at least one methanotrophic species at a population frequency of at least 0.01% for at least 1, 2, 3, 4, 5, 6, 8, 10, 12, or more weeks. In certain embodiments, one or more additives are in a sufficient amount to keep at least one methanotrophic species at a population frequency of at least 0.1% for at least 1, 2, 3, 4, 5, 6, 8, 10, 12, or more weeks. In certain embodiments, one or more additives are in a sufficient amount to keep at least one methanotrophic species at a population frequency of at least 1% for at least 1, 2, 3, 4, 5, 6, 8, 10, 12, or more weeks. In certain embodiments, one or more additives are in a sufficient amount to keep at least one methanotrophic species at a population frequency of at least 10% for at least 1, 2, 3, 4, 5, 6, 8, 10, 12, or more weeks.

In some embodiments, one or more additives are in an amount sufficient to favor fixation of N₂. In certain embodiments, one or more additives are in a sufficient amount to keep at least one diazotroph species at a population frequency of at least 0.01% for at least 1, 2, 3, 4, 5, 6, 8, 10, 12, or more weeks. In certain embodiments, one or more additives are in a sufficient amount to keep at least one diazotroph species at a population frequency of at least 0.1% for at least 1, 2, 3, 4, 5, 6, 8, 10, 12, or more weeks. In certain embodiments, one or more additives are in a sufficient amount to keep at least one diazotroph species at a population frequency of at least 1% for at least 1, 2, 3, 4, 5, 6, 8, 10, 12, or more weeks. In certain embodiments, one or more additives are in a sufficient amount to keep at least one diazotroph species at a population frequency of at least 10% for at least 1, 2, 3, 4, 5, 6, 8, 10, 12, or more weeks.

In some embodiments, one or more additives may promote consumption of CH₄ and fixation of N₂ by the consortium of bacteria. In various embodiments, one or more additives may promote consumption of CH₄ by the consortium of bacteria, but may not promote fixation of N₂. In certain embodiments, one or more additives may promote fixation of N₂ by the consortium of bacteria but may not promote consumption of CH₄.

In various embodiments, one or more additives may promote consumption of CH₄ by the consortium of bacteria by about 5% to about 100%. In various embodiments, one or more additives may promote consumption of CH₄ by the consortium of bacteria by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In various embodiments, one or more additives may promote consumption of CH₄ by the consortium of bacteria by about 5%, about 10%, about 20%, about 30%, about 40%, about 50%, about 60%, about 70%, about 80%, about 90%, or about 100%. In various embodiments, one or more additives may promote consumption of CH₄ by the consortium of bacteria by at least about 5%, about 10%, about 20%, about 30%, about 40%, about 50%, about 60%, about 70%, about 80%, or about 90%. In various embodiments, one or more additives may promote consumption of CH₄ by the consortium of bacteria by at most about 10%, about 20%, about 30%, about 40%, about 50%, about 60%, about 70%, about 80%, about 90%, or about 100%.

In various embodiments, one or more additives may promote consumption of CH₄ by the consortium of bacteria by about 1 fold to about 10,000 fold. In various embodiments, one or more additives may promote consumption of CH₄ by the consortium of bacteria by about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 200 fold, about 1 fold to about 500 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 200 fold, about 2 fold to about 500 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 200 fold, about 5 fold to about 500 fold, about 5 fold to about 1,000 fold, about 5 fold to about 10,000 fold, about 10 fold to about 20 fold, about 10 fold to about 50 fold, about 10 fold to about 100 fold, about 10 fold to about 200 fold, about 10 fold to about 500 fold, about 10 fold to about 1,000 fold, about 10 fold to about 10,000 fold, about 20 fold to about 50 fold, about 20 fold to about 100 fold, about 20 fold to about 200 fold, about 20 fold to about 500 fold, about 20 fold to about 1,000 fold, about 20 fold to about 10,000 fold, about 50 fold to about 100 fold, about 50 fold to about 200 fold, about 50 fold to about 500 fold, about 50 fold to about 1,000 fold, about 50 fold to about 10,000 fold, about 100 fold to about 200 fold, about 100 fold to about 500 fold, about 100 fold to about 1,000 fold, about 100 fold to about 10,000 fold, about 200 fold to about 500 fold, about 200 fold to about 1,000 fold, about 200 fold to about 10,000 fold, about 500 fold to about 1,000 fold, about 500 fold to about 10,000 fold, or about 1,000 fold to about 10,000 fold. In various embodiments, one or more additives may promote consumption of CH₄ by the consortium of bacteria by about 1 fold, about 2 fold, about 5 fold, about 10 fold, about 20 fold, about 50 fold, about 100 fold, about 200 fold, about 500 fold, about 1,000 fold, or about 10,000 fold. In various embodiments, one or more additives may promote consumption of CH₄ by the consortium of bacteria by at least about 1 fold, about 2 fold, about 5 fold, about 10 fold, about 20 fold, about 50 fold, about 100 fold, about 200 fold, about 500 fold, or about 1,000 fold. In various embodiments, one or more additives may promote consumption of CH₄ by the consortium of bacteria by at most about 2 fold, about 5 fold, about 10 fold, about 20 fold, about 50 fold, about 100 fold, about 200 fold, about 500 fold, about 1,000 fold, or about 10,000 fold.

In various embodiments, one or more additives may promote fixation of N₂ by the consortium of bacteria by about 5% to about 100%. In various embodiments, one or more additives may promote fixation of N₂ by the consortium of bacteria by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In various embodiments, one or more additives may promote fixation of N₂ by the consortium of bacteria by about 5%, about 10%, about 20%, about 30%, about 40%, about 50%, about 60%, about 70%, about 80%, about 90%, or about 100%. In various embodiments, one or more additives may promote fixation of N₂ by the consortium of bacteria by at least about 5%, about 10%, about 20%, about 30%, about 40%, about 50%, about 60%, about 70%, about 80%, or about 90%. In various embodiments, one or more additives may promote fixation of N₂ by the consortium of bacteria by at most about 10%, about 20%, about 30%, about 40%, about 50%, about 60%, about 70%, about 80%, about 90%, or about 100%.

In various embodiments, one or more additives may promote fixation of N₂ by the consortium of bacteria by about 1 fold to about 10,000 fold. In various embodiments, one or more additives may promote fixation of N₂ by the consortium of bacteria by about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 200 fold, about 1 fold to about 500 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 200 fold, about 2 fold to about 500 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 200 fold, about 5 fold to about 500 fold, about 5 fold to about 1,000 fold, about 5 fold to about 10,000 fold, about 10 fold to about 20 fold, about 10 fold to about 50 fold, about 10 fold to about 100 fold, about 10 fold to about 200 fold, about 10 fold to about 500 fold, about 10 fold to about 1,000 fold, about 10 fold to about 10,000 fold, about 20 fold to about 50 fold, about 20 fold to about 100 fold, about 20 fold to about 200 fold, about 20 fold to about 500 fold, about 20 fold to about 1,000 fold, about 20 fold to about 10,000 fold, about 50 fold to about 100 fold, about 50 fold to about 200 fold, about 50 fold to about 500 fold, about 50 fold to about 1,000 fold, about 50 fold to about 10,000 fold, about 100 fold to about 200 fold, about 100 fold to about 500 fold, about 100 fold to about 1,000 fold, about 100 fold to about 10,000 fold, about 200 fold to about 500 fold, about 200 fold to about 1,000 fold, about 200 fold to about 10,000 fold, about 500 fold to about 1,000 fold, about 500 fold to about 10,000 fold, or about 1,000 fold to about 10,000 fold. In various embodiments, one or more additives may promote fixation of N₂ by the consortium of bacteria by about 1 fold, about 2 fold, about 5 fold, about 10 fold, about 20 fold, about 50 fold, about 100 fold, about 200 fold, about 500 fold, about 1,000 fold, or about 10,000 fold. In various embodiments, one or more additives may promote fixation of N₂ by the consortium of bacteria by at least about 1 fold, about 2 fold, about 5 fold, about 10 fold, about 20 fold, about 50 fold, about 100 fold, about 200 fold, about 500 fold, or about 1,000 fold. In various embodiments, one or more additives may promote fixation of N₂ by the consortium of bacteria by at most about 2 fold, about 5 fold, about 10 fold, about 20 fold, about 50 fold, about 100 fold, about 200 fold, about 500 fold, about 1,000 fold, or about 10,000 fold.

In some embodiments, one or more additives may comprise a metal. In some embodiments, one or more additives may comprise a metallic salt. In some embodiments, an additive may comprise copper, tungsten, iron, nickel, molybdenum, cerium, manganese, zinc, cobalt, boron, one or more lanthanides, or a combination thereof. In some embodiments, a lanthanide may comprise lanthanum, cerium, praseodymium, neodymium, promethium, samarium, europium, gadolinium, terbium, dysprosium, holmium, erbium, thulium, ytterbium, lutetium, or any combination thereof. In various embodiments, the one or more additive comprises copper. In some embodiments, the metal additives are components of the enzymes required for methane consumption or nitrogen fixation.

In some embodiments of the present disclosure, the additives may be added to the media used to grow the consortium of bacteria or certain species in the consortium of bacteria. Non-limiting example of some embodiments of additives may comprises MgSO₄·7H₂O, CaCl₂·6H₂O, NaCl, KH₂PO₄, Na₂CO₃, Na₂-EDTA, FeSO₄·7H₂O, ZnSO₄·7H₂O, MnCl₂·4H₂O, H₃BO₃, CoCl₂·6H₂O, CuCl₂·2H₂O, NiCl₂·6H₂O, or Na₂MoO₄·2H₂O.

In certain embodiments, some additives affect the growth rate and the amount of lipids produced by a particular bacterium. In some embodiments, it can be desirable to have a concentration of Cu⁺⁺ in the media or environment of at least 7.5 mol/gDW. In some embodiments, the amount of copper may eventually become toxic to the bacterium. In some embodiments, a concentration of Cu⁺² in the media or environment may be within a range of about 7 M to about 20 M. In certain embodiments, a concentration of Fe⁺² in the media or environment may be within the range of about 5 M to about 15 M. In some embodiments, a concentration of NO₃⁻ in the media or environment may vary from about 5 mmol/gDW to about 8 mmol/gDW. Additionally, in some embodiments, a concentration of PO₄-3 in the media or environment may range from about 1.4 mmol/gDW to about 2 mmol/gDW. In certain embodiments, one or more additive is present in the consortium of bacteria at a concentration of at least about 1 ug/L, about 10 ug/L, about 100 ug/L, or about 1 mg/L. In some embodiments, nitrogen may be provided at a concentration of at least 4 mM.

In certain embodiments, additives may be added separately and at different concentrations or molarity. In certain embodiments, additives may be added separately and at similar concentrations or molarity. In some embodiments, additives may be added at different time points. In some embodiments, additives may be added at the same time point. In various embodiments, the concentration of one or more additives that are added to the consortium may change. In some embodiments, the concentration of one or more additives that are added to the consortium may not change.

In some embodiments, one additive may be added to a liquid media containing the consortium of bacteria at a concentration (ug additive/L) of about 0.001 ug/L to about 100,000 ug/L. In certain embodiments, one additive may be added to a liquid media containing the consortium of bacteria at a concentration (ug additive/L) of about 0.001 ug/L to about 0.01 ug/L, about 0.001 ug/L to about 0.1 ug/L, about 0.001 ug/L to about 1 ug/L, about 0.001 ug/L to about 10 ug/L, about 0.001 ug/L to about 100 ug/L, about 0.001 ug/L to about 1,000 ug/L, about 0.001 ug/L to about 10,000 ug/L, about 0.001 ug/L to about 100,000 ug/L, about 0.01 ug/L to about 0.1 ug/L, about 0.01 ug/L to about 1 ug/L, about 0.01 ug/L to about 10 ug/L, about 0.01 ug/L to about 100 ug/L, about 0.01 ug/L to about 1,000 ug/L, about 0.01 ug/L to about 10,000 ug/L, about 0.01 ug/L to about 100,000 ug/L, about 0.1 ug/L to about 1 ug/L, about 0.1 ug/L to about 10 ug/L, about 0.1 ug/L to about 100 ug/L, about 0.1 ug/L to about 1,000 ug/L, about 0.1 ug/L to about 10,000 ug/L, about 0.1 ug/L to about 100,000 ug/L, about 1 ug/L to about 10 ug/L, about 1 ug/L to about 100 ug/L, about 1 ug/L to about 1,000 ug/L, about 1 ug/L to about 10,000 ug/L, about 1 ug/L to about 100,000 ug/L, about 10 ug/L to about 100 ug/L, about 10 ug/L to about 1,000 ug/L, about 10 ug/L to about 10,000 ug/L, about 10 ug/L to about 100,000 ug/L, about 100 ug/L to about 1,000 ug/L, about 100 ug/L to about 10,000 ug/L, about 100 ug/L to about 100,000 ug/L, about 1,000 ug/L to about 10,000 ug/L, about 1,000 ug/L to about 100,000 ug/L, or about 10,000 ug/L to about 100,000 ug/L. In various embodiments, one additive may be added to a liquid media containing the consortium of bacteria at a concentration (ug additive/L) of about 0.001 ug/L, about 0.01 ug/L, about 0.1 ug/L, about 1 ug/L, about 10 ug/L, about 100 ug/L, about 1,000 ug/L, about 10,000 ug/L, or about 100,000 ug/L. In some embodiments, one additive may be added to a liquid media containing the consortium of bacteria at a concentration (ug additive/L) of at least about 0.001 ug/L, about 0.01 ug/L, about 0.1 ug/L, about 1 ug/L, about 10 ug/L, about 100 ug/L, about 1,000 ug/L, or about 10,000 ug/L. In certain embodiments, one additive may be added to a liquid media containing the consortium of bacteria at a concentration (ug additive/L) of at most about 0.01 ug/L, about 0.1 ug/L, about 1 ug/L, about 10 ug/L, about 100 ug/L, about 1,000 ug/L, about 10,000 ug/L, or about 100,000 ug/L.

In various embodiments, one additive may be added to a liquid or solid composition of about 0.001 umol to about 50,000 umol. In some embodiments, one additive may be added to a liquid or solid composition of about 0.001 umol to about 0.01 umol, about 0.001 umol to about 0.1 umol, about 0.001 umol to about 1 umol, about 0.001 umol to about 10 umol, about 0.001 umol to about 50 umol, about 0.001 umol to about 100 umol, about 0.001 umol to about 500 umol, about 0.001 umol to about 1,000 umol, about 0.001 umol to about 5,000 umol, about 0.001 umol to about 10,000 umol, about 0.001 umol to about 50,000 umol, about 0.01 umol to about 0.1 umol, about 0.01 umol to about 1 umol, about 0.01 umol to about 10 umol, about 0.01 umol to about 50 umol, about 0.01 umol to about 100 umol, about 0.01 umol to about 500 umol, about 0.01 umol to about 1,000 umol, about 0.01 umol to about 5,000 umol, about 0.01 umol to about 10,000 umol, about 0.01 umol to about 50,000 umol, about 0.1 umol to about 1 umol, about 0.1 umol to about 10 umol, about 0.1 umol to about 50 umol, about 0.1 umol to about 100 umol, about 0.1 umol to about 500 umol, about 0.1 umol to about 1,000 umol, about 0.1 umol to about 5,000 umol, about 0.1 umol to about 10,000 umol, about 0.1 umol to about 50,000 umol, about 1 umol to about 10 umol, about 1 umol to about 50 umol, about 1 umol to about 100 umol, about 1 umol to about 500 umol, about 1 umol to about 1,000 umol, about 1 umol to about 5,000 umol, about 1 umol to about 10,000 umol, about 1 umol to about 50,000 umol, about 10 umol to about 50 umol, about 10 umol to about 100 umol, about 10 umol to about 500 umol, about 10 umol to about 1,000 umol, about 10 umol to about 5,000 umol, about 10 umol to about 10,000 umol, about 10 umol to about 50,000 umol, about 50 umol to about 100 umol, about 50 umol to about 500 umol, about 50 umol to about 1,000 umol, about 50 umol to about 5,000 umol, about 50 umol to about 10,000 umol, about 50 umol to about 50,000 umol, about 100 umol to about 500 umol, about 100 umol to about 1,000 umol, about 100 umol to about 5,000 umol, about 100 umol to about 10,000 umol, about 100 umol to about 50,000 umol, about 500 umol to about 1,000 umol, about 500 umol to about 5,000 umol, about 500 umol to about 10,000 umol, about 500 umol to about 50,000 umol, about 1,000 umol to about 5,000 umol, about 1,000 umol to about 10,000 umol, about 1,000 umol to about 50,000 umol, about 5,000 umol to about 10,000 umol, about 5,000 umol to about 50,000 umol, or about 10,000 umol to about 50,000 umol. In some embodiments, one additive may be added to a liquid or solid composition at a molarity of about 0.001 umol, about 0.01 umol, about 0.1 umol, about 1 umol, about 10 umol, about 50 umol, about 100 umol, about 500 umol, about 1,000 umol, about 5,000 umol, about 10,000 umol, or about 50,000 umol. In certain embodiments, one additive may be added to a liquid or solid composition of at least about 0.001 umol, about 0.01 umol, about 0.1 umol, about 1 umol, about 10 umol, about 50 umol, about 100 umol, about 500 umol, about 1,000 umol, about 5,000 umol, or about 10,000 umol. In various embodiments, one additive may be added to a liquid or solid composition of at most about 0.01 umol, about 0.1 umol, about 1 umol, about 10 umol, about 50 umol, about 100 umol, about 500 umol, about 1,000 umol, about 5,000 umol, about 10,000 umol, or about 50,000 umol.

In various embodiments, one additive may be added to a liquid or solid composition at a molarity of about 0.001 uM to about 50,000 uM. In some embodiments, one additive may be added to a liquid or solid composition at a molarity of about 0.001 uM to about 0.01 uM, about 0.001 uM to about 0.1 uM, about 0.001 uM to about 1 uM, about 0.001 uM to about 10 uM, about 0.001 uM to about 50 uM, about 0.001 uM to about 100 uM, about 0.001 uM to about 500 uM, about 0.001 uM to about 1,000 uM, about 0.001 uM to about 5,000 uM, about 0.001 uM to about 10,000 uM, about 0.001 uM to about 50,000 uM, about 0.01 uM to about 0.1 uM, about 0.01 uM to about 1 uM, about 0.01 uM to about 10 uM, about 0.01 uM to about 50 uM, about 0.01 uM to about 100 uM, about 0.01 uM to about 500 uM, about 0.01 uM to about 1,000 uM, about 0.01 uM to about 5,000 uM, about 0.01 uM to about 10,000 uM, about 0.01 uM to about 50,000 uM, about 0.1 uM to about 1 uM, about 0.1 uM to about 10 uM, about 0.1 uM to about 50 uM, about 0.1 uM to about 100 uM, about 0.1 uM to about 500 uM, about 0.1 uM to about 1,000 uM, about 0.1 uM to about 5,000 uM, about 0.1 uM to about 10,000 uM, about 0.1 uM to about 50,000 uM, about 1 uM to about 10 uM, about 1 uM to about 50 uM, about 1 uM to about 100 uM, about 1 uM to about 500 uM, about 1 uM to about 1,000 uM, about 1 uM to about 5,000 uM, about 1 uM to about 10,000 uM, about 1 uM to about 50,000 uM, about 10 uM to about 50 uM, about 10 uM to about 100 uM, about 10 uM to about 500 uM, about 10 uM to about 1,000 uM, about 10 uM to about 5,000 uM, about 10 uM to about 10,000 uM, about 10 uM to about 50,000 uM, about 50 uM to about 100 uM, about 50 uM to about 500 uM, about 50 uM to about 1,000 uM, about 50 uM to about 5,000 uM, about 50 uM to about 10,000 uM, about 50 uM to about 50,000 uM, about 100 uM to about 500 uM, about 100 uM to about 1,000 uM, about 100 uM to about 5,000 uM, about 100 uM to about 10,000 uM, about 100 uM to about 50,000 uM, about 500 uM to about 1,000 uM, about 500 uM to about 5,000 uM, about 500 uM to about 10,000 uM, about 500 uM to about 50,000 uM, about 1,000 uM to about 5,000 uM, about 1,000 uM to about 10,000 uM, about 1,000 uM to about 50,000 uM, about 5,000 uM to about 10,000 uM, about 5,000 uM to about 50,000 uM, or about 10,000 uM to about 50,000 uM. In some embodiments, one additive may be added to a liquid or solid composition at a molarity of about 0.001 uM, about 0.01 uM, about 0.1 uM, about 1 uM, about 10 uM, about 50 uM, about 100 uM, about 500 uM, about 1,000 uM, about 5,000 uM, about 10,000 uM, or about 50,000 uM. In certain embodiments, one additive may be added to a liquid or solid composition at a molarity of at least about 0.001 uM, about 0.01 uM, about 0.1 uM, about 1 uM, about 10 uM, about 50 uM, about 100 uM, about 500 uM, about 1,000 uM, about 5,000 uM, or about 10,000 uM. In various embodiments, one additive may be added to a liquid or solid composition at a molarity of at most about 0.01 uM, about 0.1 uM, about 1 uM, about 10 uM, about 50 uM, about 100 uM, about 500 uM, about 1,000 uM, about 5,000 uM, about 10,000 uM, or about 50,000 uM.

In some embodiments, the additives may be added to a liquid media containing the consortium of bacteria to yield a total additive concentration (ug additive/L) of about 0.1 ug/L to about 100,000 ug/L. In some embodiments, the additives may be added to a liquid media containing the consortium of bacteria to yield a total additive concentration (ug additive/L) of about 0.1 ug/L to about 1 ug/L, about 0.1 ug/L to about 10 ug/L, about 0.1 ug/L to about 100 ug/L, about 0.1 ug/L to about 1,000 ug/L, about 0.1 ug/L to about 10,000 ug/L, about 0.1 ug/L to about 100,000 ug/L, about 1 ug/L to about 10 ug/L, about 1 ug/L to about 100 ug/L, about 1 ug/L to about 1,000 ug/L, about 1 ug/L to about 10,000 ug/L, about 1 ug/L to about 100,000 ug/L, about 10 ug/L to about 100 ug/L, about 10 ug/L to about 1,000 ug/L, about 10 ug/L to about 10,000 ug/L, about 10 ug/L to about 100,000 ug/L, about 100 ug/L to about 1,000 ug/L, about 100 ug/L to about 10,000 ug/L, about 100 ug/L to about 100,000 ug/L, about 1,000 ug/L to about 10,000 ug/L, about 1,000 ug/L to about 100,000 ug/L, or about 10,000 ug/L to about 100,000 ug/L. In some embodiments, the additives may be added to a liquid media containing the consortium of bacteria to yield a total additive concentration (ug additive/L) of about 0.1 ug/L, about 1 ug/L, about 10 ug/L, about 100 ug/L, about 1,000 ug/L, about 10,000 ug/L, or about 100,000 ug/L. In various embodiments, the additives may be added to a liquid media containing the consortium of bacteria to yield a total additive concentration (ug additive/L) of at least about 0.1 ug/L, about 1 ug/L, about 10 ug/L, about 100 ug/L, about 1,000 ug/L, or about 10,000 ug/L. In certain embodiments, the additives may be added to a liquid media containing the consortium of bacteria to yield a total additive concentration (ug additive/L) of at most about 1 ug/L, about 10 ug/L, about 100 ug/L, about 1,000 ug/L, about 10,000 ug/L, or about 100,000 ug/L.

In certain embodiments, the additive may be added to a liquid or solid composition to yield a total additive molarity of about 0.001 uM to about 50,000 uM. In some embodiments, the additive may be added to a liquid or solid composition to yield a total additive molarity of about 0.001 uM to about 0.01 uM, about 0.001 uM to about 0.1 uM, about 0.001 uM to about 1 uM, about 0.001 uM to about 10 uM, about 0.001 uM to about 50 uM, about 0.001 uM to about 100 uM, about 0.001 uM to about 500 uM, about 0.001 uM to about 1,000 uM, about 0.001 uM to about 5,000 uM, about 0.001 uM to about 10,000 uM, about 0.001 uM to about 50,000 uM, about 0.01 uM to about 0.1 uM, about 0.01 uM to about 1 uM, about 0.01 uM to about 10 uM, about 0.01 uM to about 50 uM, about 0.01 uM to about 100 uM, about 0.01 uM to about 500 uM, about 0.01 uM to about 1,000 uM, about 0.01 uM to about 5,000 uM, about 0.01 uM to about 10,000 uM, about 0.01 uM to about 50,000 uM, about 0.1 uM to about 1 uM, about 0.1 uM to about 10 uM, about 0.1 uM to about 50 uM, about 0.1 uM to about 100 uM, about 0.1 uM to about 500 uM, about 0.1 uM to about 1,000 uM, about 0.1 uM to about 5,000 uM, about 0.1 uM to about 10,000 uM, about 0.1 uM to about 50,000 uM, about 1 uM to about 10 uM, about 1 uM to about 50 uM, about 1 uM to about 100 uM, about 1 uM to about 500 uM, about 1 uM to about 1,000 uM, about 1 uM to about 5,000 uM, about 1 uM to about 10,000 uM, about 1 uM to about 50,000 uM, about 10 uM to about 50 uM, about 10 uM to about 100 uM, about 10 uM to about 500 uM, about 10 uM to about 1,000 uM, about 10 uM to about 5,000 uM, about 10 uM to about 10,000 uM, about 10 uM to about 50,000 uM, about 50 uM to about 100 uM, about 50 uM to about 500 uM, about 50 uM to about 1,000 uM, about 50 uM to about 5,000 uM, about 50 uM to about 10,000 uM, about 50 uM to about 50,000 uM, about 100 uM to about 500 uM, about 100 uM to about 1,000 uM, about 100 uM to about 5,000 uM, about 100 uM to about 10,000 uM, about 100 uM to about 50,000 uM, about 500 uM to about 1,000 uM, about 500 uM to about 5,000 uM, about 500 uM to about 10,000 uM, about 500 uM to about 50,000 uM, about 1,000 uM to about 5,000 uM, about 1,000 uM to about 10,000 uM, about 1,000 uM to about 50,000 uM, about 5,000 uM to about 10,000 uM, about 5,000 uM to about 50,000 uM, or about 10,000 uM to about 50,000 uM. In some embodiments, the additive may be added to a liquid or solid composition to yield a total additive molarity of about 0.001 uM, about 0.01 uM, about 0.1 uM, about 1 uM, about 10 uM, about 50 uM, about 100 uM, about 500 uM, about 1,000 uM, about 5,000 uM, about 10,000 uM, or about 50,000 uM. In various embodiments, the additive may be added to a liquid or solid composition to yield a total additive molarity of at least about 0.001 uM, about 0.01 uM, about 0.1 uM, about 1 uM, about 10 uM, about 50 uM, about 100 uM, about 500 uM, about 1,000 uM, about 5,000 uM, or about 10,000 uM. In certain embodiments, the additive may be added to a liquid or solid composition to yield a total additive molarity of at most about 0.01 uM, about 0.1 uM, about 1 uM, about 10 uM, about 50 uM, about 100 uM, about 500 uM, about 1,000 uM, about 5,000 uM, about 10,000 uM, or about 50,000 uM.

In some embodiments of the present disclosure, the media used to grow methane-consuming bacteria may comprise at least one exogenous nitrogen source such as KNO₃, NaNO₃, NH₄Cl, (NH₄)₂SO₄, urea, or mixtures thereof. In certain embodiments, exogenous nitrogen refers to non-atmospheric nitrogen readily available in the soil, field, or growth medium and may comprise ammonia, ammonium, nitrate, nitrite, urea, uric acid, ammonium acids, or a combination thereof. In further embodiments of the present disclosure, media used to grow one or more bacteria of the bacterial consortium may comprise at least one nitrogen source comprising KNO₃, NaNO₃, or mixtures thereof. In some embodiments of the present disclosure, the media used to grow methane-consuming bacteria may lack an exogenous nitrogen source. In some embodiments, the media used to grow methane-consuming bacteria is nitrogen-free. In some embodiments, an exogenous form of nitrogen is not required to support the growth or viability of the consortium.

In some embodiments, one or more additives comprises at least one non-metal additive. In certain embodiments, the at least one non-metal additive comprises phosphorus, ethylenediaminetetraacetic acid, or a combination thereof. In some embodiments, an additive may comprise malic acid.

In some embodiments, the additives may be added to the consortium of bacteria in a range of different conditions. In some embodiments, the additives may be added to the consortium of bacteria in ambient outdoor conditions. In some embodiments, the additives may be added to the consortium of bacteria in field conditions. In certain embodiments, the additives may be added to the soil prior to, after, or at the same time as the consortium of bacteria is added to the soil. In certain embodiments, the additives may be added to the consortium of bacteria in a compost reactor or bioreactor. In some embodiments, the additives may be added to the consortium of bacteria in greenhouse conditions. In some embodiments the additives may be added to the consortium of bacteria in laboratory conditions. In some embodiments, the additives may be added to the consortium of bacteria at sea level. In some embodiments, the additives may be added to the consortium of bacteria at elevation. In certain embodiments, the additives may be added to the consortium of bacteria more frequently or less frequently at elevations above sea level. In certain embodiments, the additives may be added to the consortium of bacteria more frequently or less frequently at elevations below sea level.

In some embodiments, the additives may be added to growth media, soil, fertilizer, compost, biomass, or any other aqueous solution or solid that houses the consortium of bacteria. In some embodiments, the additives may be added to a material before, after, or at the same time as the consortium of bacteria is introduced to said material. In some embodiments, the additives may be added to the bacterial consortium before or after the consortium of bacteria is freeze-dried, spray-frozen, lyophilized, or refrigerated. In some embodiments, the additives improve the methane consumption and nitrogen fixation capability of the bacterial consortium following freeze-dry, spray-freeze, lyophilization, or refrigeration.

In some embodiments, additives may be added to the bacterial consortium one time. In some embodiments, additives may be added to the bacterial consortium more than one time. In some embodiments, additives may be added to the bacterial consortium at least 1 time, at least 2 times, at least 3 times, at least 4 time, 5 times, at least 6 times, at least 7 times, at least 8 times, at least 9 times, at least 10 times, at least 15 times, at least 20 times, at least 25 times, at least 30 times, at least 35 times, at least 40 times, at least 45 times, at least 50 times, at least 60 times, at least 70 times, at least 80 times, at least 90 times, at least 100 times, at least 150 times, or at least 200 times.

In some embodiments, one additive may be added to the bacterial consortium. In some embodiments, more than one additive may be added to the bacterial consortium. In some embodiments, more than one additive may be added to the bacterial consortium and each additive is added at the same frequency. In some embodiments, more than one additive may be added to the bacterial consortium and a subset of additives may be added at a different frequency than other additives. In some embodiments, all additives are added to the bacterial consortium at different frequencies. In some embodiments, the frequency of adding an additive to the bacterial consortium may be at least every hour, at least every 2 hours, at least every 3 hours, at least every 4 hours, at least every 5 hours, at least every 6 hours, at least every 7 hours, at least every 8 hours, at least every 9 hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, at least every 24 hours, at least every day, at least every 2 days, at least every 3 days, at least every 4 days, at least every 5 days, at least every 6 days, at least every 7 days, at least every 8 days, at least every 9 days, at least every 10 days, at least every 15 days, at least every 20 days, at least every 30 days, at least every month, at least every 2 months, at least every 3 months, at least every 4 months, at least every 5 months, at least every 6 months, at least every 7 months, at least every 8 months, at least every 9 months, at least every 10 months, at least every 11 months, or at least every 12 months. In some embodiments, the additives may be added to the bacterial consortium at the same time as the addition of additional bacteria. In some embodiments, the frequency of adding an additive may change over time.

### V. Method of Enrichment

A method of enriching for one or more bacteria is disclosed herein. In some embodiments, the method of enriching one or more bacteria comprises introducing to a composition a consortium of bacteria comprising the one or more bacteria. In some embodiments, the consortium of bacteria is introduced to one or more additives. In various embodiments, the additive comprises a metal. In some embodiments, the consortium of bacteria consumes CH₄ at a rate of at least 5 umol/gDW per day. In some embodiments, the consortium of bacteria consumes CH₄ at a rate of at least 10, 15, 20, 25, 30, 35, or more umol/gDW per day. In some embodiments, the consortium of bacteria consumes N₂ at a rate of at least 2.0 umol/gDW per day. In some embodiments, the consortium of bacteria consumes N₂ at a rate greater than 1.0, 1.5, 2.0, 2.5, 3.0, 3.5, 4.0, or more umol/gDW per day.

In some embodiments, the method disclosed herein may enrich for a consortium of bacteria that may comprise 1, 2, 3, 4, 5, 6, 7, 8, 9, 10, or more species from Tables 1, 3, or 4. In certain embodiments, the method disclosed herein may enrich for a consortium of bacteria that may comprise one or more species from Tables 1, 3, or 4 in combination with other bacteria that may consume methane only, fix nitrogen only, or do neither consume methane nor fix nitrogen. In various embodiments, the method disclosed herein may enrich for a consortium of bacteria that may comprise only bacteria listed in Tables 1, 3, or 4.

In certain embodiments, an additive may comprise a metal. In some embodiments, an additive may comprise copper, tungsten, iron, nickel, molybdenum, cerium, manganese, zinc, cobalt, boron, one or more lanthanides, or a combination thereof. In some embodiments, a lanthanide may comprise lanthanum, cerium, praseodymium, neodymium, promethium, samarium, europium, gadolinium, terbium, dysprosium, holmium, erbium, thulium, ytterbium, lutetium, or any combination thereof. In certain embodiments, the metal additive comprises copper. In some embodiments, the metal is present in the consortium of bacteria at a concentration of at least about 1 ug/L, about 10 ug/L, about 100 ug/L, or about 1 mg/L.

In certain embodiments, the additive may further comprise magnesium sulfate heptahydrate (MgSO₄·7H₂O), potassium nitrate (KNO₃), calcium chloride dihydrate (CaCl₂·2H₂O), monopotassium phosphate (KH₂PO₄), dibasic potassium phosphate (K₂HPO₄), sodium molybdate dihydrate (Na₂MoO₄·2H₂O), copper(II) sulfate pentahydrate (CuSO₄·5H₂O), zinc sulfate heptahydrate (ZnSO₄·7H₂O), manganese(II) chloride tetrahydrate (MnCl₂·4H₂O), cobalt(II) chloride hexahydrate (CoCl₂·H₂O), nickel(II) chloride hexahydrate (NiCl₂·6H₂O), boric acid (H₃BO₃), ethylenediaminetetraacetic acid (EDTA), ethylenediaminetetraacetic acid ferric sodium salt, or a combination thereof.

In certain embodiments, the one or more bacteria are increased by at least about 10%, 20%, 30%, 40% or 50% as compared to a reference composition that comprises the consortium of bacteria and lacking the additive. In certain embodiments, the one or more bacteria are increased by at least about 10%, 20%, 30%, 40% or 50% as compared to a reference composition that comprises the consortium of bacteria and lacking the additive one month, two months, four months, six months, or twelve months after the introducing. In various embodiments, the one or more bacteria are provided with the additive at least once every 12 hours, once per day, once per week, twice per month, once per month, once every 2 months, or once every 6 months.

In some embodiments, the one or more bacteria that is enrichened comprises a methanotroph. In some embodiments, one or more methanotroph is enriched to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the population in a consortium. In some embodiments, the bacteria being enriched can consume methane are enriched 5%, 10%, 20%, 30%, 40%, 50%, 60%, 70%, 80%, 90%, 100%, 125%, 150%, 175%, 200%, 500%, or more over bacteria that cannot consume methane. In certain embodiments, the methane-consuming bacteria are enriched over 1, 2, 3, 4, 5, 6, 7, or more days. In certain embodiments, the methane-consuming bacteria are enriched over 1, 2, 3, 4, 5, 6, 7, 8, 9, 10, 11, 12, or more weeks. In certain embodiments, the methane-consuming bacteria are enriched over 1, 2, 3, 4, 5, 6, 7, 8, 9, 10, 11, 12, or more months.

In certain embodiments, the one or more bacteria being enriched comprises a nitrogen fixing bacteria. In some embodiments, one or more diazotroph is enriched to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the population in a consortium. In some embodiments, the bacteria being enriched can fix nitrogen are enriched 5%, 10%, 20%, 30%, 40%, 50%, 60%, 70%, 80%, 90%, 100%, 125%, 150%, 175%, 200%, 500%, or more over bacteria that cannot fix nitrogen. In certain embodiments, the nitrogen-fixing bacteria are enriched over 1, 2, 3, 4, 5, 6, 7, or more days. In certain embodiments, the nitrogen-fixing bacteria are enriched over 1, 2, 3, 4, 5, 6, 7, 8, 9, 10, 11, 12, or more weeks. In certain embodiments, the nitrogen-fixing bacteria are enriched over 1, 2, 3, 4, 5, 6, 7, 8, 9, 10, 11, 12, or more months. In some embodiments, the one or more bacteria comprises at least one bacterial species listed in Tables 1, 3, or 4.

In certain embodiments, the one or more bacteria being enriched comprises a bacteria that is capable of consuming methane and fixing nitrogen. In some embodiments, one or more bacteria that is capable of consuming methane and fixing nitrogen is enriched to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 96%, 97%, 98%, 99%, or 100% of the population in a consortium. In some embodiments, the one or more bacteria that is capable of consuming methane and fixing nitrogen are enriched 5%, 10%, 20%, 30%, 40%, 50%, 60%, 70%, 80%, 90%, 100%, 125%, 150%, 175%, 200%, 500%, or more over bacteria that cannot consume methane and fix nitrogen. In certain embodiments, the one or more bacteria that is capable of consuming methane and fixing nitrogen are enriched over 1, 2, 3, 4, 5, 6, 7, or more days. In certain embodiments, the one or more bacteria that is capable of consuming methane and fixing nitrogen are enriched over 1, 2, 3, 4, 5, 6, 7, 8, 9, 10, 11, 12, or more weeks. In certain embodiments, the one or more bacteria that is capable of consuming methane and fixing nitrogen are enriched over 1, 2, 3, 4, 5, 6, 7, 8, 9, 10, 11, 12, or more months. In some embodiments, the one or more bacteria comprises a bacteria listed in Tables 1, 3, or 4.

In various embodiments, the bacterial consortium may be grown in nitrogen-free media, compost, or soil. In some embodiments, the bacterial consortium may be grown in media, compost, or soil that is substantially nitrogen-free. In some embodiments, the bacterial consortium may be grown in media, compost, or soil that is essentially nitrogen-free. In various embodiments, the bacterial consortium may be grown in media, compost, or soil that comprises about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 3.5%, or about 4.0% nitrogen. In some embodiments, the bacterial consortium may be grown in media, compost, or soil that comprises less than about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 3.5%, or about 4.0% nitrogen. In certain embodiments, the bacterial consortium may be grown in media, compost, or soil that comprises at least about 0.1%, about 0.2%, about 0.3%, about 0.4%, about 0.5%, about 0.6%, about 0.7%, about 0.8%, about 0.9%, about 1.0%, about 1.5%, about 2.0%, about 2.5%, about 3.0%, about 3.5%, or about 4.0% nitrogen. In certain embodiments, the bacterial consortium grown in nitrogen-free media, compost, or soil, may fix enough nitrogen to support the growth and survival of non-nitrogen fixing bacteria.

In some embodiments, a composition comprises a liquid composition. In various embodiments, the composition comprises soil. In certain embodiments, the composition comprises compost. In certain embodiments, the consortium of bacteria may be added to a composition. In some embodiments, the consortium of bacteria may be added to a composition, nonlimiting examples of which might include soil, compost, media, plants, plant roots, plant leaves, plant stems, clay, gravel, or any combination thereof. The enrichment of the consortium of bacteria disclosed herein may be enriched in any potential growth mediums, nonlimiting examples include liquid culture, solid culture, agar, soil, compost, plant roots, plant stems or leaves, or any other medium suitable for bacteria. In some embodiments, the enrichment selects for beneficial bacteria. In some embodiments, the enrichment kills and/or outcompetes undesired bacteria that may be present in the soil, compost, plant, or other composition. In some embodiments, the enrichment selects for beneficial bacteria and kills and/or outcompetes undesired bacteria that may be present in the soil, compost, plant, or other composition.

In some embodiments, the process of enrichment comprises introducing additives and/or removing components in the environment of the consortium. The environment of the consortium of bacteria may comprise the medium in which the consortium of bacteria is grown, the gas or air interacting with the consortium of bacteria, material added to the consortium, or any other condition that may influence the consortium of bacteria. Nonlimiting examples of the environment that may influence the consortium of bacteria comprises temperature, altitude, exposure to sunlight, local ecosystems (such as existing microbiomes in soil or water, animal excrement or mechanical disruption), or pollution.

In some embodiments, the method of enrichment also removes undesirable bacterium in the consortium. In some embodiments, an undesirable bacterium that is present in the composition prior to the introduction of additives or enrichment method is undetectable about 1, 2, 3, 4, 5, 6, 7, 8, 9, 10, 11, 12, or more months following the enrichment. In certain embodiments, an undesirable bacterium that is present in the composition prior to the introduction of additives or enrichment method is undetectable about 1, 2, 3, 4, 5, 6, 7, 8, 9, 10, 11, 12, or more weeks following the enrichment. In some embodiments, the method of enrichment may reduce growth or proliferation of bacteriophages. In some embodiments, the method of enrichment may reduce growth or proliferation of bacteriophages that infect one or more bacteria in the bacterial consortium. In certain embodiments, the method of enrichment may reduce or minimize infection of one or more of the bacteria in the bacterial consortium.

In various embodiments, the enrichment of the consortium of bacteria provides selection for bacteria that can consume methane. In various embodiments, the enrichment of the consortium of bacteria provides selection for bacteria that can fix nitrogen. In various embodiments, the enrichment of the consortium of bacteria provides selection for bacteria that can consume methane and fix nitrogen. In some embodiments, the enrichment of the consortium of bacteria provides selection for bacteria that have symbiotic relationships with the one or more bacteria that can fix nitrogen or consume methane. In some embodiments, the enrichment of the consortium of bacteria provides selection for bacteria that support the growth and viability of the one or more bacteria that can fix nitrogen or consume methane.

In some embodiments, following the introducing, the composition is exposed to ambient air. In some embodiments, the bacterial consortium may be exposed to hypoxia conditions. In some embodiments, the bacterial consortium may be exposed to methane. In some embodiments, the bacterial consortium may be exposed to additives as disclosed herein as part of the enrichment process.

In certain embodiments, the enrichment process may yield a bacterial consortium that consumes more than 10, 15, 20, 25, 30, 35, or more umol CH₄/gDW per day and more than 1.0, 1.5, 2.0, 2.5, 3.0, 3.5, 4.0, or more umol N₂/gDW per day. In certain embodiments, the enrichment process may yield a bacterial consortium that consumes more than 10, 15, 20, 25, 30, 35, and more umol CH₄/gDW per day or more than 1.0, 1.5, 2.0, 2.5, 3.0, 3.5, 4.0, or more umol N₂/gDW per day.

In certain embodiments, the enriched consortium improves the quality of soil when applied to soil. In certain embodiments, the enriched consortium improves the growth and survival of plants when applied to the plant or the soil in which the plant grows.

### VI. Compost

In various embodiments, a compost composition as disclosed herein may comprise a conversion of organic materials into nutrient-rich soil amendment or mulch. In some embodiments, a compost composition may comprise the consortium of bacteria as described herein. In some embodiments, a compost composition may be inoculated by an embodiment of the consortium of bacteria as described herein. In certain embodiments, a compost composition comprising the consortium of bacteria disclosed herein may provide compost that is more nutritious to soil than commercially available or home-grown compost that does not comprise the bacterial consortium.

In various embodiments, a compost may comprise a biomass, water, and oxygen. In certain embodiments, a biomass may comprise organic matter, such as but not limited to: cow manure, manure or excrement from other animals, food scraps, leaves, plant material, untreated woody material, coffee grounds, grass clippings, vegetable and fruit peels, egg shells, cardboard, manure, bone meal, bird cage waste, corn cobs, feathers, fish scraps, flowers, ground bones, hair, hay, insects, leather, natural fibers such as cotton, linen, or wool, newspaper, paper, peanut shells, pine needles, potting soil, rice, saw dust, tobacco, vacuum bag wasters, and twigs.

In some embodiments, the compost may be generated by introducing a biomass to the bacterial consortium provided herein. In certain embodiments, the biomass may be inoculated with the consortium of bacteria at one timepoint, or at multiple time points. In certain embodiments, the biomass may be inoculated with the consortium of bacteria every week, month, or year. In certain embodiments, the compost may be inoculated with at least 10³, 10⁴, 10¹, 10⁶, 10⁷, 10⁸, 10⁹, 10¹⁰, 10¹¹, 10¹², 10¹³, 10¹⁴, or 10¹⁵ colony forming units (CFUs) of the consortium of bacteria. In some embodiments, the compost may be inoculated by a bacterial consortium that is lyophilized, spray dried, freeze dried, in liquid form, in gel form, in a matrix, on or in a biocurtain, or on or in a cover. In various embodiments, the compost may be inoculated by a bacterial consortium that is suspended in culture. The bacterial consortium may be introduced to most or all of the compost pile, a portion of the compost pile, or to a vent or area that ventilates methane from the pile. The bacterial consortium may be introduced onto the top of the compost, in the middle of the compost, on the bottom of the compost, a combination thereof, or on specific sections of the compost. The bacterial consortium may also be introduced in, in proximity to, or on a ventilation component or ventilation pipe that ventilates and/or aerates the compost pile. In some embodiments, additives may be added to the compost. In some embodiments, additives may be added to the compost at the time of inoculation or at timepoints thereafter. In some embodiments, additives may be added to the compost every day, week, or month. The additives may be introduced to most or all of the compost pile, a portion of the compost pile, or to a vent or area that ventilates methane. The additives may be introduced onto the top of the compost, in the middle of the compost, on the bottom of the compost, a combination thereof, or on specific sections of the compost. The additives may also be introduced in, in proximity to, or on a ventilation component or ventilation pipe that ventilates and/or aerates the compost pile.

In some embodiments, the compost following inoculation is enhanced with nitrogen compared to a reference compost that is substantially identical to the compost except the reference compost does not comprise the consortium of bacteria. In certain embodiments, the inoculated compost consumes methane at a rate that is higher compared to a reference compost that is substantially identical to the compost except the reference compost does not comprise the consortium of bacteria.

In various embodiments, the inoculated compost is maintained outside in ambient air. In certain embodiments, the inoculated compost is exposed to temperatures ranging from about −20° C. to over 50° C.

In certain embodiments, the inoculated compost is applied to soil. When applied to soil, the inoculated compost may improve the ability of the soil to hold nutrients. In some embodiments, the inoculated compost may improve a soil's cation exchange capacity when applied to soil. In some embodiments, the inoculated compost may provide nitrogen, phosphorus, and potassium to soil and any plants grown in such soil. In various embodiments, the inoculated compost improves nitrogen availability of soil when applied to the soil. In some embodiments, the inoculated compost may comprise sulfur, carbon, magnesium, calcium, copper, tungsten, iron, nickel, molybdenum, cerium, manganese, zinc, cobalt, boron, one or more lanthanides, or a combination thereof. In some embodiments, a lanthanide may comprise lanthanum, cerium, praseodymium, neodymium, promethium, samarium, europium, gadolinium, terbium, dysprosium, holmium, erbium, thulium, ytterbium, lutetium, or any combination thereof.

In certain embodiments, the inoculated compost composition disclosed herein may comprise a minimum amount of nitrogen. In some embodiments, the compost may provide at least 0.1%, 0.5%, 1.0%, 1.5%, 2.0%, or 2.5% nitrogen. In some embodiments, the compost may provide at least 10%, 11%, 12%, 13%, 14%, 15%, 16%, 17%, or more nitrogen. In some embodiments, the compost may provide nitrogen in the form of ammonia, ammonium, nitrates, nitrites, or any combination thereof. In some embodiments, the compost may provide nitrogen in the form of amino acids, proteins, putrescine, cadaverine, glucosamine, aminated carbohydrates, other organic nitrogen-containing biomolecules, or any combination thereof.

In some embodiments, the compost may be mixed routinely. In some embodiments, the compost may be mixed multiple times a week, once a week, once every two weeks, once every three weeks, once every four weeks, once every 8 weeks, or once every 12 weeks. In some embodiments, the compost may be maintained at a moisture level suitable to promote decomposition of organic matter. In certain embodiments, the compost pile may be moist enough to prevent the compost from catching fire. In various embodiments, the compost pile can include macroorganisms including insects and other microorganisms.

In certain embodiments the compost pile may be poorly maintained. In some embodiments, a compost pile that is poorly maintained can include too much water or moisture, include too much nitrogen, lack nitrogen, lack carbon, have an imbalance between nitrogen and carbon, have poor aeration, be unmixed or poorly mixed, be burnt, lack microbes or other life forms, be too compressed, be otherwise present in poor conditions, or a combination thereof. In some embodiments, the compost may be maintained in poor conditions before being inoculated with the consortium of bacteria. In some embodiments, the compost may be maintained in poor conditions after being inoculated with the consortium of bacteria. In some embodiments, the compost can be maintained in poor conditions both before and after being inoculated with the consortium of bacteria.

A compost may be inoculated with the consortium of bacteria as disclosed herein as well as with a biochar. In some embodiments, biochar comprises carbon that can be made from organic waste material or biomass. In some cases, the biochar may comprise organic waste material or biomass that is partially combusted in the presence of limited oxygen or in the absence of oxygen. In certain examples, the biochar may be produced through pyrolysis, or the thermos-chemical conversion of dry organic materials. The biochar may comprise wood, wood chips, timbe slash, leaf, leaf litter, dead plants, feedstock, cornstalks, manure, other biomass, or a combination thereof. In some cases, biochar can be used as a soil amendment, by itself, or blended with other soil amendments, such as compost. The biochar may be infused with the bacterial consortium. In some embodiments, the compost is infused with the biochar that is infused with the bacterial consortium. In some embodiments, the compost is infused with the biochar and is also infused with the bacterial consortium. The biochar may improve soil health, bind heavy metals and chemicals, raise soil pH, remediate polluted soils, sequester carbon, lower greenhouse gas emissions, and improve soil moisture. In some embodiments, the use of biochar may improve crops treated with the compost, biochar, and consortium of bacteria.

### Compost Reactor

In some embodiments, the compost is maintained in a compost reactor. In certain embodiments, the methane oxidation and nitrogen fixation can occur in a windrow or vessel. In some embodiments, methane or a mixture of ambient air and methane may be blown through a manifold. In certain embodiments, the methane or a mixture of ambient air and methane may distribute the gas into a compost substrate that has been inoculated with the consortium. In various embodiments, the compost inoculated with the consortium is monitored. In one nonlimiting example, moisture monitoring may control the addition of liquid/additives. In some embodiments, the addition of liquid or additives may occur on the top of or into the compost in the reactor. An example of a similar method may be described as “static aerated pile” compost.

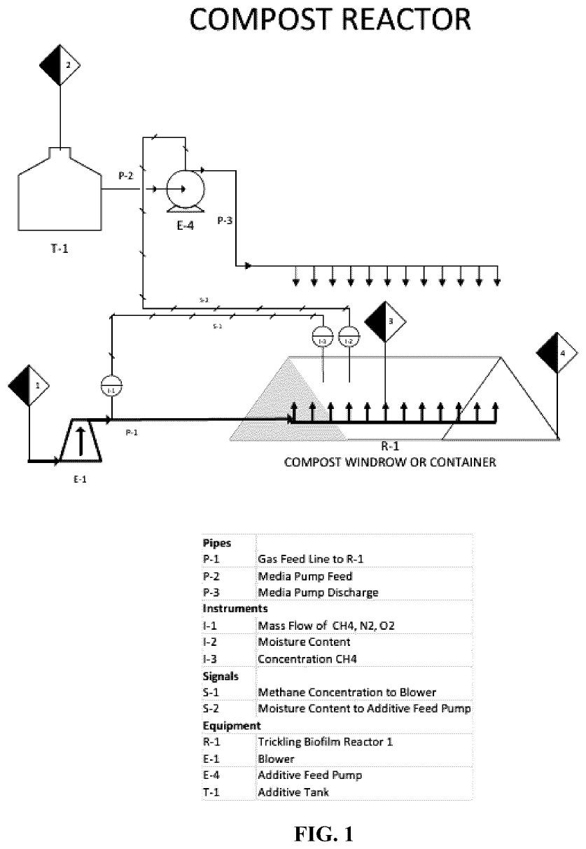

In some embodiments, the compost reactor may provide a windrow or container for which to hold compost. In one nonlimiting example, an embodiment of a compost windrow or container may resemble R-1 in FIG. 1. In some embodiments, the compost reactor may provide components that support the health and viability of the bacterial consortium disclosed herein, similar to that depicted in P-1 (FIG. 1). In some cases, the compost reactor provides a gas line that may provide a carbon source to the compost inoculated with the consortium. In some cases, the compost reactor provides a gas line that may provide an oxygen source to the compost inoculated with the consortium. In some cases, the compost reactor provides a gas line may provide a nitrogen source to the compost inoculated with the consortium. In some cases, the compost reactor provides a gas line may provide both a carbon and oxygen source to the compost inoculated with the consortium. In some cases, the compost reactor may provide a gas line that provides a carbon, oxygen, nitrogen source to the compost inoculated with the consortium through a single delivery mechanism. In certain cases, a gas line may deliver ambient air to the compost inoculated with the consortium. In certain cases, a gas line may deliver air from a natural or industrial source to the compost inoculated with the consortium. In various cases, a gas line may deliver concentrated air to the compost inoculated with the consortium. In some embodiments, the gas line may be coupled to a blower, such as that depicted in E-1 (FIG. 1).

In some embodiments, there may be an instrument that might be configured to measure the mass flow of CH₄, N₂, and 02 being delivered to the compost. In some embodiments, there may be an instrument that might be configured to measure the mass flow of CH₄, N₂, or 02 being delivered to the compost. In certain embodiments, an instrument that measures the mass flow CH₄, N₂, or O₂ may resemble I-1 as depicted in FIG. 1.

In some embodiments, the compost reactor may further comprise instruments that enable monitoring of different features of the compost. In certain embodiments, the compost reactor may include an instrument configured to measure a concentration of CH₄, a nonlimiting example of which is depicted as I-3 in FIG. 1. In some cases, the instrument configured to measure a concentration of CH₄ may measure a concentration of CH₄ at one or more time points. In certain instances, the instrument may measure a concentration of CH₄ and provide feedback to the gas line, similar to that depicted as S-1 in FIG. 1. The feedback provided by the instrument configured to measure CH₄ may cause a change in the flow of gas to the compost. In some embodiments, the flow of gas may be increased or decreased depending on the concentration of CH₄ infiltrating the compost pile.

In some cases, the compost reactor may comprise an instrument configured to monitor moisture of the compost. In certain embodiments, the instrument configured to monitor moisture of the compost may resemble I-2 depicted in FIG. 1. In various embodiments, the instrument configured to measure moisture content may measure moisture at one or more time points. The instrument configured to measure moisture may provide feedback to an additive feed pump. The additive pump, in some embodiments, is configured to connect to an additive tank, similar to that depicted as T-1 in FIG. 1. In certain embodiments, the additives tank may be configured to communicate with the additive feed pump in a configuration similar to the depicted by P-2 in FIG. 1. In certain embodiments, the additives may be distributed to the compost as a liquid, gas, or solid. In some cases, the additives may be distributed to the compost in a fashion that is similar to that depicted by P-3 in FIG. 1.

In some embodiments, the additive tank may comprise reagents. In some embodiments, the additives may support the growth and viability of the consortium. In certain embodiments, the additives from the additive tank may be provided to the compost based on the feedback of the instrument configured to measure moisture. In some embodiments, the additive tank may comprise the additives disclosed herein that improve the viability of methane-consuming bacteria. In certain embodiments, the additive tank may comprise one or more additives disclosed herein that improve the methane consumption of the bacterial consortium. In certain embodiments, the additive tank may comprise one or more additives disclosed herein that improves the nitrogen fixation by the bacterial consortium. In certain embodiments, the additive tank may comprise one or more additives that improved the methane consumption and nitrogen fixation of the bacterial consortium disclosed herein. In some embodiments, the additives tank may comprise additives suspended in an aqueous solution. In certain aspects, the additives tank may comprise any additive that supports or improves the health of one or more bacteria at equilibrium or following any chemical, biological, or physical disruption.

In some embodiments, one or more additives may promote consumption of CH₄ and fixation of N₂ by the consortium of bacteria. In various embodiments, one or more additives may promote consumption of CH₄ by the consortium of bacteria, but may not promote fixation of N₂. In certain embodiments, one or more additives may promote fixation of N₂ by the consortium of bacteria but may not promote consumption of CH₄.

In some embodiments, one or more additives may comprise a metal. In some embodiments, one or more additives may comprise a metallic salt. In some embodiments, an additive may comprise copper, tungsten, iron, nickel, molybdenum, cerium, manganese, zinc, cobalt, boron, one or more lanthanides, or a combination thereof. In some embodiments, a lanthanide may comprise lanthanum, cerium, praseodymium, neodymium, promethium, samarium, europium, gadolinium, terbium, dysprosium, holmium, erbium, thulium, ytterbium, lutetium, or any combination thereof. In various embodiments, the one or more additive comprises copper. In some embodiments, the metal additives are components of the enzymes required for methane consumption or nitrogen fixation.

In some embodiments of the present disclosure, the additives may be added to the media used to grow the consortium of bacteria or certain species in the consortium of bacteria. Non-limiting example of some embodiments of additives may comprises MgSO₄·7H₂O, CaCl₂·6H₂O, NaCl, KH₂PO₄, Na₂CO₃, Na₂-EDTA, FeSO_{4·}7H₂O, ZnSO₄·7H₂O, MnCl₂·4H₂O, H₃BO₃, CoCl₂·6H₂O, CuCl₂·2H₂O, NiCl₂·6H₂O, and Na₂MoO₄·2H₂O.

In certain embodiments, some additives affect the growth rate and the amount of lipids produced by a particular bacterium. In some embodiments, it can be desirable to have a concentration of Cu⁺⁺ in the media or environment of at least 7.5 mol/gDW. Since the amount of copper may eventually become toxic to the bacterium. In some embodiments, a concentration of Cu⁺⁺ in the media or environment may be within a range of about 7 M to about 20 M. In certain embodiments, a concentration of Fe⁺⁺ in the media or environment may be within a range of about 5 M to about 15 M. In some embodiments, a concentration of NO₃⁻ in the media or environment may vary from about 5 mmol/gDW to about 8 mmol/gDW. In some embodiments, a concentration PO₄⁻³ in the media or environment may range from about 1.4 mmol/gDW to about 2 mmol/gDW. In certain embodiments, one or more additive is present in the consortium of bacteria at a concentration of at least about 1 ug/L, about 10 ug/L, about 100 ug/L, or about 1 mg/L. In some embodiments, nitrogen is provided at a concentration of at least 4 mM.

In some embodiments of the present disclosure, the media used to grow methane-consuming bacteria may comprise at least one exogenous nitrogen source such as KNO₃, NaNO₃, NH₄Cl, (NH₄)₂SO₄, urea, or mixtures thereof. In certain embodiments, exogenous nitrogen refers to non-atmospheric nitrogen readily available in the soil, field, or growth medium and may comprise ammonia, ammonium, nitrate, nitrite, urea, uric acid, ammonium acids, or a combination thereof. In further embodiments of the present disclosure, media used to grow one or more bacteria of the bacterial consortium may comprise at least one nitrogen source comprising KNO₃, NaNO₃, or mixtures thereof. In some embodiments of the present disclosure, the media used to grow methane-consuming bacteria may lack an exogenous nitrogen source. In some embodiments, the media used to grow methane-consuming bacteria is nitrogen-free. In some embodiments, an exogenous form of nitrogen is not required to support to the growth and viability of the consortium.

In some embodiments, one or more additives comprises at least one non-metal additive. In certain embodiments, the at least one non-metal additive comprises phosphorus, ethylenediaminetetraacetic acid, or a combination thereof. In some embodiments, an additive may comprise malic acid.

### VII. Treating Crops

The compositions comprising the bacterial consortium described herein can be used to improve agricultural compositions, crops, or other plants. In some embodiments, the bacterial consortium described herein may improve plant traits by improving the soil condition or the soil in which the plants are grown. In some embodiments, the soil may be in a confined structure, such as a pot, bowl, cup, jar, mug, box, bottle, or any other structure. In some embodiments, the soil is in a field, on a plot of land, on a landfill, near a body of water, in a superfund site, or in any other outdoor environment. In some embodiments, the bacterial consortium described herein may improve the soil condition by increasing the nitrogen in the soil. In some embodiments, the bacterial consortium described herein may improve the soil condition by using metals found in the soil.

In some embodiments, the bacterial consortium described herein may improve plant traits by improving nitrogen in the soil. The growth of all plants may be determined directly or indirectly by the availability of mineral nutrients, especially nitrogen. Often, once water needs are met, an important limiting factor is nitrogen. A plant with nitrogen deficiency may suffer chlorosis, manifesting a yellowish coloration of stems and leaves, lack of development and weakness. On the contrary, when the plant has enough nitrogen, its leaves and stems can grow rapidly. In agriculture, nitrogen is the main nutrient for the growth of plants and, thus, in soils lacking in nitrogen, crop yields are low. Nitrogen is available as N₂ in the atmosphere, however, due to the nature of the bond between the two nitrogen atoms, atmospheric nitrogen is often in the form of N₂, its reactivity is nearly zero, which means that it is not assimilable by most living organisms. Microorganisms, such as one of more bacteria in an embodiment of the consortium of bacteria disclosed herein, may fix nitrogen through the conversion of atmospheric nitrogen into metabolizable forms that can be assimilated by living beings, such as, for example, ammonium forms, nitrites and nitrates. In some embodiments, one or more species of the bacterial consortium disclosed herein may fix nitrogen from the atmosphere and convert it into biologically available forms for use by plants. In certain embodiments, the bacterial composition disclosed herein may provide nitrogen to the soil. In various embodiments, an increase in nitrogen in the soil may improve plant and crops growth and health.

In some embodiments, the compositions comprising the bacterial consortium described herein can improve plant traits, such as promoting plant growth, maintaining high chlorophyll content in leaves, increasing fruit or seed numbers, and increasing fruit or seed unit weight. Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, or proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation.

In some embodiments, soil inoculated with the bacterial consortium improves viability and water content of plants, maintaining the chlorophyll content and chlorophyll a/b ratio, and promote root growth. Certain strains may fix and transform aerial nitrogen, synthesize siderophores that promote iron-uptake from the soil, or solubilize insoluble or poorly soluble minerals, providing e.g. available phosphorus supply for plants. These effects may contribute to the efficiency and the speeding up of the process of revitalizing soils.

The compositions comprising the bacterial consortium described herein can be used to reduce methane produced by a crop or plant, during the process of growing the crop or plant, or during the process of harvesting the crop or plant. In some embodiments, the bacterial consortium described herein may consume methane that is produced during the growing process of a plant or crop. In some embodiments, the bacterial consortium described herein may consume the methane produced by methane-producing bacteria that grow around, in, or on a plant or crop. In some embodiments, the bacterial consortium may consume methane in the soil or water in which the plant or crop is grown. In various embodiments, the bacterial consortium may consume methane in or from the stems, leaves, roots, or other components of the crop or plant. In some embodiments, the bacterial consortium disclosed herein can consume methane in the air around, in, or above the area in which the crops or plants are grown.

A soil or the medium in which a crop or plant grows may be inoculated with the consortium of bacteria as disclosed herein as well as with a biochar. In some embodiments, biochar comprises carbon that can be made from organic waste material or biomass. In some cases, the biochar may comprise organic waste material or biomass that is partially combusted in the presence of limited oxygen or in the absence of oxygen. In certain examples, the biochar may be produced through pyrolysis, or the thermo-chemical conversion of dry organic materials. The biochar may comprise wood, wood chips, timber slash, leaf, leaf litter, dead plants, feedstock, cornstalks, manure, other biomass, or a combination thereof. In some cases, biochar can be used as a soil amendment, by itself, or blended with other soil amendments, such as compost. The biochar may improve soil health, bind heavy metals and chemicals, raise soil pH, remediate polluted soils, sequester caron, lower greenhouse gas emissions, and improve soil moisture. In some embodiments, the use of biochar may improve the crops treated with the biochar and the consortium of bacteria.

### VIII. Bioreactor

The disclosure provided herein may comprise a system in some embodiments. In certain embodiments, a system may comprising a reaction chamber, a first fluid input in fluid communication with the reaction chamber, wherein the first fluid input is configured to direct gas enriched in methane (CH₄) into the reaction chamber, a second fluid input in fluid communication with the reaction chamber, wherein the second fluid input is configured to direct the consortium of bacteria as described herein into the reaction chamber; and an outlet in fluid communication with the reaction chamber. In some embodiments, the first fluid input and the second fluid input may be the same input. In various embodiments, the first fluid input and the second fluid input may be different from one another.

In some embodiments, the enriched CH₄ is at a concentration of at least 1.89 parts per million (ppm). In certain embodiments, the enriched CH₄ is from a storage of concentrated CH₄. In some embodiments, enriched CH₄ is from an animal housing or free-range ruminants. In various embodiments, the enriched CH₄ is from any industrial source. Non-limiting examples of industrial sources may include a natural gas pipeline, a syngas reactor, a reactor, or a refinery. In some embodiments, the enriched CH₄ is from any natural source.

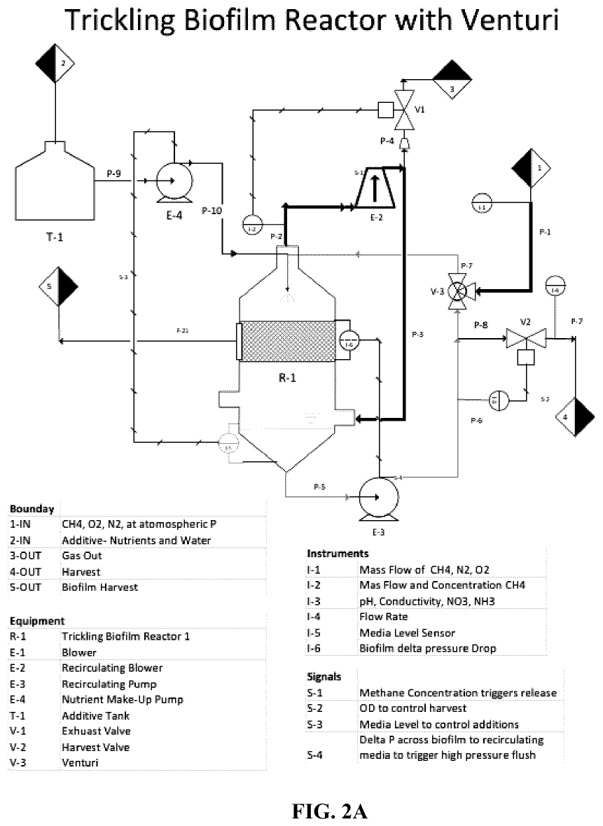

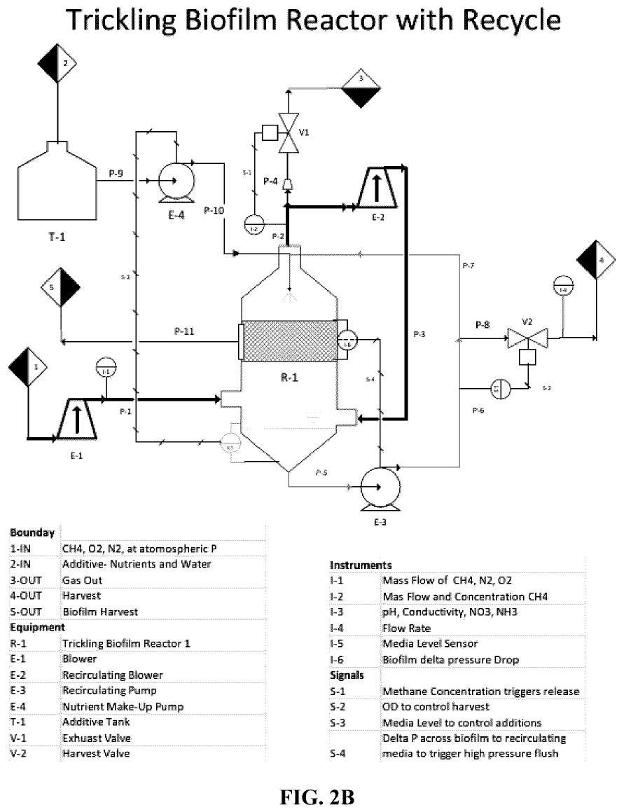

In certain embodiments, the system may comprise a bio trickling filter (BTF). A nonlimiting example of an embodiment of a similar reactor may be found as FIG. 2A and FIG. 2B. In some embodiments, the reactor may comprise a tower with suspended matrix with water trickling over it (R-1 in FIGS. 2A and 2B). In some embodiments, the matrix may comprise a biocurtain, a gel, a scaffolding, or another matrix. In some embodiments, the matrix may support bacterial growth or provide a surface on which the bacteria can grow. The matrix may be inoculated with the consortium of bacteria as described herein. In some examples, the matrix may comprise the additives. In certain examples, the additives are added directly to the matrix or may be supplied to the consortium of bacteria through a water, liquid, powder, spray, or solid supply. In some embodiments, the reactor is purposed for biomass. In certain embodiments, the liquid may circulate over a biofilm or biocurtain. In some embodiments, the circulated liquid may be harvested. In certain embodiments, the biofilm or biocurtain itself may be harvested. In this example, the circulating water may collect in the bottom of the tower. Accumulation of the water in the bottom of the tower may comprise accumulation of elevated concentrations of the bacteria and nitrogen dissolved in the water or captured as the biofilm or biocurtain sluffs off the matrix.

In certain embodiments, the system may dissolve methane, oxygen, or some other nutrient into trickling water. In some embodiments, the Venturi (V-3, FIG. 2A) may dissolve methane into the trickling water. In other embodiments, a blower may circulate gaseous compounds (E-1, FIG. 2B). In some cases, the reactor may comprise one or more additional blowers and/or pumps to aid in the recirculation of air (E-1, E-3, FIG. 2B). The biofilm may be harvested separately from the circulating water by removing it from the suspending matrix. In some embodiments, the matrix may be biodegradable. In some embodiments, the reactor may resemble the compost reactor as disclosed herein.

### IX. Other Uses of the Consortium

The bacterial consortium can be applied to a range of natural, human-made, or industrial sources. In some cases, the bacterial consortium may be applied by an individual, a group of individuals, a company, an institution, an organization, a government, or another entity.

Non-limiting examples of natural applications include bogs, reservoirs or ponds, wildfires or instances of burning biomass, wetlands, marshes, fens, fresh water, geological seepage, wild animals, termites, permafrost, vegetation, decomposition, dams, or the ocean.

Potential human-made applications for the bacterial consortium as disclosed here in can include farms, dairy farms, barns, gardens, composts, indoor or outdoor plants, wells, agriculture fields, agriculture plants, crops (non-limiting examples of which may include Kharif crops, rice, corn or maize, soybeans, barley, oats, wheat, sorghum, grain sorghum, grain, alfalfa, hay, millet, flax, tobacco, cotton, sugar or sugarcane, sweeteners, rye, coffee beans, cocoa, peanuts, walnuts, almonds, tree nuts, legumes, peas, beans, mustard, sunflower, canola, oil crops, vegetables (e.g., peppers, potatoes, sweet potatoes, tomatoes, cabbage, lettuce, beets, kale, spinach, chilies, garlic, onions, horseradish, Japanese horseradish or wasabi, celery, cauliflower and broccoli, cucumbers, leeks, shallots, chives, squash, zucchini, or carrots), fruits (e.g., melons, berries, apples, peaches and nectarines, pomelos, grapefruits, citrus, kiwifruit, bananas, grapes, avocados, pineapple, papaya, mango, cherries, apricots, or guava), or spices (e.g., basil, rosemary, sage, thyme, parsley, oregano, cinnamon, ginger, black pepper, star anise, turmeric, mint, saffron, oregano, or coriander)), transportation, entertainment, or in the home.

Industrial applications for which the bacterial consortium as disclosed herein can be applied may include landfills, oil or gas refineries, waste management, waste water treatment, municipal composting, municipal wells, processing plants, chemical processing facilities, exhausts, mines, food processing, food harvesting, mass transit, construction, or in manufacturing. In some embodiments, an industrial application can include any facility or infrastructure that produces, emits, or collects methane.

The bacterial composition as disclosed here can be applied to consume methane from a range of sources. Nonlimiting examples of natural sources of methane may include biogas, agriculturally-generated methane, dairy farms, ambient air, compost gas, livestock emissions, crop gas, or any combination thereof. In some embodiments, the industrial source of methane may be selected from the group consisting of, but not limited to, natural gas, synthetic natural gas, natural gas hydrates, stranded natural gas, shale gas, flared gas, coal mine methane, coal bed methane, methane produced from catalytic cracking of olefins or organic matter, landfill gas, associated petroleum gas, oil refineries, municipal compost, industrial compost, agricultural harvesting or processing, food processing, shipping, or methane produced as an unwanted by product from CO hydrogenation and hydrogenolysis reactions.

### X. Packaging, Storage, and Application

Compositions comprising bacteria or bacterial populations produced according to methods described herein and/or having characteristics as described herein can be in the form of a liquid, a foam, a gel, a matrix, or a dry product. Compositions comprising bacteria or bacterial populations produced according to methods described herein and/or having characteristics as described herein may also be used to improve compost quality, soil condition, or plant traits. In some examples, a composition comprising bacterial populations may be in the form of a dry powder, a slurry of powder and water, or a flowable treatment.

The composition can be fabricated in bioreactors such as continuous stirred tank reactors, batch reactors, and on the farm or other location where methane is available. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case. The composition can be spray dried, freeze dried, or vacuum sealed. The composition can be stored in a stab culture or in a culture comprising glycerol. In certain embodiments, packaging may be sterilized prior to introduction of the consortium.

The bacterial species may be present in compositions at a concentration of between 10⁵ to 10¹⁰ CFU/mL. In some examples, compositions may be supplemented with trace metal ions, such as molybdenum ions, iron ions, manganese ions, copper ions, zinc ions, or combinations of these ions. The concentration of ions in examples of compositions as described herein may between about 0.1 mM and about 50 mM.

In some embodiments, the composition comprising the consortium of bacteria may comprise metals. In some embodiments, an additive is selected from the group consisting of: copper, tungsten, iron, nickel, molybdenum, cerium, manganese, zinc, cobalt, boron, one or more lanthanides, or a combination thereof. In some embodiments, a lanthanide may comprise lanthanum, cerium, praseodymium, neodymium, promethium, samarium, europium, gadolinium, terbium, dysprosium, holmium, erbium, thulium, ytterbium, lutetium, or any combination thereof. The concentration of metals in examples of compositions as described herein may be between about 0.1 mM and about 50 mM.

Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat, compost, or planting materials can be used as a carrier, or biopolymers in which a composition is entrapped in the biopolymer can be used as a carrier.

In certain cases, the packaging may include a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%.

In some embodiments, storage allows for a commercially viable product, which has sufficient shelf life to allow for efficient storage, sale and distribution to customers. In some embodiments, compositions comprising bacteria or bacterial populations produced according to methods described herein and/or having characteristics as described herein can be stored at room temperature.

The bacterial consortium as disclosed herein may be applied in a variety of ways. In some cases, the bacterial consortium may be applied as a powder, a solid, a liquid, a gel, a spray, a foam, in a matrix, as a biocurtain, sheet, wrapping, infusion, film, frozen solid, or a culture. In some cases, the bacterial consortium may be infused into a matrix or material, applied onto the surface of a matrix or material, or coated or soaked into a matrix or material. In some embodiments, matrix or biocurtain may be permeable or porous. The matrix or biocurtain may be impermeable. The matrix or biocurtain may provide a structural scaffold on which the bacterial consortium can grow and/or survive. In some embodiments, the bacterial consortium may be applied in combination with a compost, a biochar, a soil amendment, a biocurtain, or a fertilizer.

In some embodiments, the bacterial consortium may be supplemented with additives before, during, or after application. The additives may be applied a powder, a solid, a liquid, a gel, a spray, a foam, in a matrix, as a biocurtain, sheet, wrapping, infusion, film, frozen solid, or a culture. The additives may be infused into a matrix or material, applied onto the surface of a matrix or material, or coated or soaked into a matrix or material.

### XI. Kit

A kit-of-parts comprising a bacterial composition together with instructions for use is further provided. For convenience, the kit-of-parts may comprise additives or reagents in predetermined amounts with instructions for use.

In some embodiments, disclosed herein are kits comprising a bacterial consortium disclosed herein. In some embodiments, a kit can be a starter culture kit. In some embodiments, a kit comprises a bacterial composition disclosed herein and instructions for use. In some embodiments, a kit comprises means for consuming CH₄ and fixing N₂ using a bacterial consortium and instructions for use. A kit may provide a unit or device for obtaining a sample (e.g., forceps).

A kit may include a plurality of syringes, ampules, foil packets, or blister packs, each containing a single unit dose of a kit component described herein. Containers of a kit may be airtight, waterproof (e.g., impermeable to changes in moistures or evaporation), and/or light-tight. A kit may include a device suitable for administration of the components, e.g., a syringe, inhalant, pipette, forceps, measured spoon, dropper (e.g., eye dropper), swab (e.g., a cotton swab or wooden swab), or any such delivery device. A kit disclosed herein may comprise one or more reagents or instruments which enable the method to be carried out. In some embodiments, reagents or instruments include one or more of the following: suitable media or buffers (aqueous solutions), additives to support viability of the bacterial consortium, or additives to improve CH₄ consumption or N₂ fixation of the bacterial consortium. A kit may be a specific kit for a specific consortium. Further, a kit disclosed herein may comprise a control. In some embodiments, the kit may comprise any of the compositions (e.g., bacterial consortium) described herein which includes any of bacteria described herein. In some embodiments, a kit can include a solid composition (e.g., a lyophilized, freeze-dried, or spray dried composition including any of the bacteria or additives described herein) and a liquid for solubilizing the lyophilized composition. In some embodiments, the kit includes a vial including any of the bacterial consortium compositions described herein (e.g., formulated as a culture, fertilizer, or compost composition).

In addition to the above components, instructions for use may be provided in a kit. These instructions may be presented in the kit in a variety of forms, such as printed information on a suitable medium or substate (e.g., a piece or pieces of paper on which the information is printed), in the packaging of the kit, in a package insert, etc. In some embodiments, instructions for use can be provided on a computer readable medium (e.g., jump/thumb drive, CD, etc.), or which the information has been recorded, or at a website address which may be used via the internet to access the information at a website.

### XII. Examples

The following examples are included for illustrative purposes only and are not intended to limit the scope of the disclosure.

### Example 1: Distribution of Bacterial Consortium in Compost Over Time

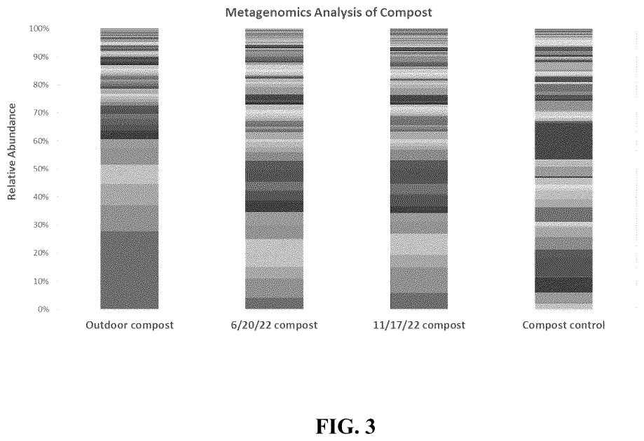

To determine the species distribution of the bacterial consortium in a compost over time, metagenomic analysis of an outdoor compost was performed at various time points (FIG. 3). A compost was inoculated with a Methylosinus trichosporium OB3b culture (FIG. 3) and maintained in an enclosed environment with 1000-8000 ppm of methane at ambient outdoor temperature and pressure conditions. The compost was maintained in ambient outdoor conditions, and sampled at three timepoints: time 0 (3/2022), time 1 (6/20/2022), and time 2 (11/27/2022). A similar compost control sourced from the same supplier was not inoculated with the consortium of bacteria. At each time point, 1 g of the treated compost samples were sent to Zymo Research on dry ice using overnight shipping. The samples were processed and analyzed with the ZymoBIOMICS® Shotgun Metagenomic Sequencing Service for Microbiome Analysis (Zymo Research, Irvine, CA). The compost control was sequenced to determine which bacteria were present in the control (FIG. 3, “outdoor compost”).

The ZymoBIOMICS® Shotgun Metagenomic Sequencing method comprises a DNA isolation step. One of three DNA extraction kits was used depending on the sample type and sample volume. In this case, the ZymoBIOMICS®-96 MagBead DNA Kit (Zymo Research, Irvine, CA) was used to extract DNA using an automated platform.

Sequencing libraries were prepared with the Illumina® DNA Library Prep Kit (Illumina, San Diego, CA) with up to 500 ng DNA input following the manufacturer's protocol using unique dual-index 10 bp barcodes with Nextera® adapters (Illumina, San Diego, CA). All libraries were quantified with TapeStation® (Agilent Technologies, Santa Clara, CA) and then pooled in equal abundance. The final pool was quantified using qPCR. The final library was sequenced using the Illumina NovaSeq® (Illumina, San Diego, CA).

Raw sequence reads were trimmed to remove low quality fractions and adapters with Trimmomatic-0.33 (Bolger et al., 2014). Quality trimming was completed by sliding window with 6 bp window size and a quality cutoff of 20, and reads with size lower than 70 bp were removed. Antimicrobial resistance and virulence factor gene identification was performed with the DIAMOND sequence aligner (Buchfink et al., 2015). Microbial composition was profiled with MetaPhlAn4 (Blanco-Miguez et al. 2022).

The species distribution of the bacterial consortium in outdoor compost changed over time (Table 5, FIG. 3).

TABLE 5
Species distribution of the bacterial consortium in outdoor compost over time

| | Outdoor | 6/20/22 | 11/17/22 | Compost |
|---|---|---|---|---|
| Species | compost | compost | compost | control |
| Actinophytocola xinjiangensis | 0.277102 | 0.03968 | 0.057249 | 0 |
| Nocardioides luteus | 0.092169 | 0.067603 | 0.087628 | 0.000138 |
| Mycolicibacterium smegmatis | 0.075506 | 0.040553 | 0.044469 | 0.000605 |
| Mycolicibacterium hassiacum | 0.069377 | 0.097261 | 0.072988 | 0.011701 |
| Mycolicibacterium phlei | 0.058562 | 0.047861 | 0.042256 | 0.003951 |
| GGB15920 SGB24193 | 0.032637 | 0.046562 | 0.030038 | 0.021345 |
| GGB15919 SGB24192 | 0.029523 | 0.03933 | 0.022594 | 0.035054 |
| Streptomyces cellulosae | 0.022251 | 0.034049 | 0.040659 | 0.000899 |
| Promicromonospora thailandica | 0.020163 | 0.003257 | 0.003779 | 0 |
| Promicromonospora citrea | 0.019469 | 0.03051 | 0.036205 | 0.000416 |
| Rhodococcus pyridinivorans | 0.015815 | 0.041046 | 0.051823 | 0.043046 |
| Sphaerobacter thermophilus | 0.013196 | 0.032099 | 0.030831 | 0.01897 |
| GGB24563 SGB36416 | 0.011278 | 0.031211 | 0.036665 | 0.028701 |
| Methanosarcina thermophila | 0.010952 | 0.016324 | 0.011144 | 0.021388 |
| GGB65186 SGB88148 | 0.01021 | 0.018409 | 0.011773 | 0.003997 |
| Geobacillus thermodenitrificans | 0.009378 | 0.011369 | 0.013633 | 0.009151 |
| Promicromonospora kroppenstedtii | 0.009238 | 0.022205 | 0.023462 | 0 |
| Mycolicibacterium agri | 0.009188 | 0.001893 | 0.002589 | 0.000099 |
| Mesorhizobium sp J18 | 0.008905 | 0.002957 | 0.00259 | 0.001704 |
| Streptomyces sp CNQ329 | 0.008148 | 0.005947 | 0.006965 | 0 |
| Streptomyces mimosae | 0.007934 | 0.003287 | 0.007566 | 0 |
| Devosia naphthalenivorans | 0.007244 | 0.006805 | 0.005632 | 0 |
| Thermaerobacter sp FW80 | 0.006999 | 0.009034 | 0.020534 | 0.027799 |
| Thermobifida fusca | 0.006582 | 0.012051 | 0.010415 | 0.003619 |
| GGB38953 SGB55198 | 0.00641 | 0.007264 | 0.005822 | 0.017312 |
| Thermobispora bispora | 0.006338 | 0.008333 | 0.007305 | 0.020842 |
| Ureibacillus terrenus | 0.00628 | 0.009449 | 0.008621 | 0.004893 |
| Thermomonospora curvata | 0.006183 | 0.012636 | 0.010749 | 0.008797 |
| GGB45347 SGB62879 | 0.00589 | 0.019573 | 0.008378 | 0.015778 |
| Pseudonocardia sp CNS 004 | 0.005882 | 0.000108 | 0.000182 | 0 |
| Thermocrispum municipale | 0.005662 | 0.006093 | 0.005402 | 0.00121 |
| Glycomyces artemisiae | 0.005588 | 0.003718 | 0.002712 | 0 |
| Olivibacter domesticus | 0.004911 | 0.000232 | 0.000093 | 0 |
| Bordetella petrii | 0.004444 | 0.00407 | 0.00382 | 0.001106 |
| Olivibacter jilunii | 0.00441 | 0.013217 | 0.013954 | 0.000119 |
| Streptomyces sp NBRC 109706 | 0.004404 | 0.006722 | 0.007928 | 0 |
| Gordonia paraffinivorans | 0.004348 | 0.026078 | 0.022959 | 0.023633 |
| Actinotalea caeni | 0.004305 | 0.010407 | 0.011199 | 0.001616 |
| Starkeya novella | 0.003756 | 0.0048 | 0.004995 | 0 |
| Pseudonocardia thermophila | 0.003693 | 0.003917 | 0.001961 | 0.000459 |
| Planifilum fulgidum | 0.003683 | 0.005074 | 0.004873 | 0.005033 |
| Aminobacter sp J41 | 0.003294 | 0.005366 | 0.004399 | 0.009836 |
| GGB38593 SGB50207 | 0.003103 | 0.0044 | 0.001396 | 0.082511 |
| Methylocaldum szegediense | 0.003007 | 0.002651 | 0.001546 | 0.002894 |
| Bacillus coagulans | 0.00298 | 0.003818 | 0.003597 | 0.002085 |
| Jiangella alba | 0.00297 | 0 | 0 | 0 |
| Acinetobacter sp ANC 3781 | 0.002929 | 0 | 0 | 0 |
| Jiangella rhizosphaerae | 0.002817 | 0.000246 | 0.000467 | 0.000023 |
| Thermasporomyces composti | 0.002805 | 0.004326 | 0.004866 | 0.005222 |
| Cellulosimicrobium cellulans | 0.002443 | 0.007406 | 0.0065 | 0.000267 |
| Thermopolyspora flexuosa | 0.002294 | 0.002637 | 0.00279 | 0.002826 |
| Pseudorhizobium banfieldiae | 0.002076 | 0.00698 | 0.005818 | 0.00298 |
| Bacillus thermoamylovorans | 0.002069 | 0.003218 | 0.004329 | 0.000145 |
| Luteimonas sp J29 | 0.001855 | 0.013215 | 0.00872 | 0.009002 |
| Peribacillus asahii | 0.0018 | 0.001459 | 0.000956 | 0.000751 |
| Bordetella genomosp 4 | 0.001522 | 0.001231 | 0.001223 | 0.000148 |
| Bordetella genomosp 7 | 0.001497 | 0.003001 | 0.003686 | 0.002586 |
| Advenella kashmirensis | 0.001372 | 0.000689 | 0.000419 | 0 |
| Methanothermobacter marburgensis | 0.00133 | 0.001906 | 0.002087 | 0.021289 |
| Streptomyces sp SBST2 5 | 0.001278 | 0.001305 | 0.000901 | 0.000688 |
| Nitratireductor aestuarii | 0.001229 | 0.002246 | 0.001837 | 0.004059 |
| Thermobacillus composti | 0.00122 | 0.001425 | 0.001993 | 0.001365 |
| Saccharopolyspora rectivirgula | 0.001166 | 0.008189 | 0.008328 | 0.001205 |
| Saccharomonospora glauca | 0.001138 | 0.004104 | 0.003473 | 0 |
| Chelatococcus composti | 0.001136 | 0.001315 | 0.000546 | 0.006859 |
| Inquilinus limosus | 0.001081 | 0.000296 | 0.000336 | 0 |
| Ornithinimicrobium humiphilum | 0.001048 | 0.013934 | 0.018393 | 0.007767 |
| Pedobacter quisquiliarum | 0.001011 | 0.001076 | 0.0034 | 0 |
| Starkeya sp OAE902 | 0.000992 | 0.000965 | 0.001498 | 0 |
| Microlunatus parietis | 0.000988 | 0 | 0 | 0 |
| Nocardioides panzhihuensis | 0.000942 | 0.000967 | 0.000942 | 0 |
| Flavobacterium pectinovorum | 0.000927 | 0 | 0 | 0 |
| Mycolicibacterium goodii | 0.000887 | 0.000063 | 0.000116 | 0 |
| Methanothermobacter defluvii | 0.000824 | 0.001145 | 0.001314 | 0.015851 |
| Tuberibacillus calidus | 0.000822 | 0.00129 | 0.001083 | 0.001376 |
| Clostridium thermopalmarium | 0.000794 | 0.001285 | 0.001137 | 0.00038 |
| Mycetocola manganoxydans | 0.00079 | 0.004048 | 0.004131 | 0 |
| Sphingobacterium shayense | 0.000739 | 0.000885 | 0.000812 | 0.000035 |
| Pseudomonas stutzeri | 0.000737 | 0.001365 | 0.000834 | 0 |
| Acinetobacter sp ANC 4249 | 0.000727 | 0 | 0 | 0 |
| Thermocrispum agreste | 0.000654 | 0.001635 | 0.00149 | 0.001872 |
| Planifilum fimeticola | 0.000612 | 0.000775 | 0.001108 | 0.00134 |
| Parageobacillus caldoxylosilyticus | 0.000598 | 0.000528 | 0.000416 | 0.000607 |
| Rhizobium sp Root482 | 0.000594 | 0.000135 | 0.000285 | 0 |
| GGB13928 SGB21351 | 0.000571 | 0.003509 | 0.002205 | 0.001683 |
| Pusillimonas sp JR1 69 3 13 | 0.000559 | 0.000983 | 0.00051 | 0 |
| Nocardioides sp J54 | 0.000546 | 0.004608 | 0.00536 | 0.010186 |
| Paenibacillus barengoltzii | 0.000536 | 0.000984 | 0.001086 | 0.00079 |
| Microbacterium sp 1 5R | 0.000535 | 0.001192 | 0.002159 | 0 |
| Microbacterium immunditiarum | 0.000533 | 0.001098 | 0.00105 | 0.000179 |
| Methanobacterium sp MB1 | 0.000531 | 0.000515 | 0.000272 | 0.00097 |
| Streptomyces thermoviolaceus | 0.000519 | 0.000779 | 0.000907 | 0.000126 |
| Streptomyces sp SBT349 | 0.00051 | 0.00099 | 0.001489 | 0 |
| Streptomyces sp CNQ766 | 0.000493 | 0.000687 | 0.003313 | 0 |
| Microbacterium paludicola | 0.000473 | 0.005102 | 0.008895 | 0.001425 |
| Rhizobium oryzae | 0.000471 | 0.000093 | 0.000108 | 0 |
| Jiangella mangrovi | 0.000466 | 0.000013 | 0.000007 | 0 |
| Quasibacillus thermotolerans | 0.000442 | 0.000528 | 0.00048 | 0.000053 |
| Dyadobacter psychrophilus | 0.000441 | 0.000226 | 0.000116 | 0 |
| Caldibacillus debilis | 0.000429 | 0.000774 | 0.000441 | 0.000827 |
| Devosia ginsengisoli | 0.000392 | 0 | 0 | 0 |
| Algoriphagus resistens | 0.000387 | 0.000743 | 0.000296 | 0 |
| Cereibacter changlensis | 0.000385 | 0.003353 | 0.003094 | 0 |
| Mycolicibacterium thermoresistibile | 0.000361 | 0.000402 | 0.000174 | 0 |
| GGB65551 SGB88681 | 0.000357 | 0.000334 | 0.000135 | 0.007804 |
| Novosphingobium sp AP12 | 0.000332 | 0.000026 | 0 | 0 |
| Clostridium cellulosi | 0.000298 | 0.000564 | 0.000417 | 0.001512 |
| Calidifontibacillus erzurumensis | 0.000288 | 0.000428 | 0.000347 | 0.000118 |
| Bacillus sp ZZV12 4809 | 0.000274 | 0.000191 | 0.000225 | 0.000451 |
| Advenella incenata | 0.000253 | 0.000171 | 0 | 0 |
| Sphingobium baderi | 0.000219 | 0.001009 | 0.000812 | 0.000025 |
| Microbacterium lacus | 0.000213 | 0.001119 | 0.000944 | 0 |
| Curtobacterium sp PhB130 | 0.000202 | 0 | 0 | 0 |
| Microbacterium murale | 0.000198 | 0.000058 | 0.00037 | 0 |
| Mesorhizobium sp YM1C 6 2 | 0.000195 | 0.000009 | 0 | 0 |
| Devosia elaeis | 0.000191 | 0.000314 | 0.000372 | 0.000422 |
| Hansschlegelia beijingensis | 0.000191 | 0.000306 | 0.000166 | 0 |
| Albibacterium bauzanense | 0.000176 | 0 | 0 | 0 |
| Jiangella muralis | 0.000174 | 0 | 0 | 0 |
| Pseudomonas sp Bc h | 0.000173 | 0 | 0 | 0 |
| Curtobacterium sp PhB115 | 0.000164 | 0 | 0.000812 | 0 |
| Microbacterium aerolatum | 0.000159 | 0.001627 | 0.00285 | 0.000235 |
| Algoriphagus terrigena | 0.000144 | 0.000909 | 0.000354 | 0 |
| Clostridium luticellarii | 0.000134 | 0 | 0 | 0 |
| Pseudoxanthomonas suwonensis | 0.000133 | 0.001885 | 0.000837 | 0.018788 |
| Sporichthya polymorpha | 0.000121 | 0.00021 | 0.000136 | 0 |
| GGB59381 SGB81162 | 0.000117 | 0.000608 | 0.000357 | 0.005566 |
| Micrococcus luteus ATCC 49442 | 0.000108 | 0.000314 | 0.000129 | 0 |
| Pseudonocardia hierapolitana | 0.000102 | 0 | 0 | 0 |
| Fermentimonas caenicola | 0.000102 | 0.000401 | 0.000189 | 0.003588 |
| Pseudonocardia oroxyli | 0.000101 | 0 | 0 | 0 |
| Pigmentiphaga sp NML080357 | 0.000096 | 0.000144 | 0.000103 | 0 |
| Luteimonas lumbrici | 0.000091 | 0.000973 | 0.00079 | 0.000632 |
| Erwinia billingiae | 0.000089 | 0 | 0 | 0 |
| Sphingopyxis sp 113P3 | 0.000088 | 0.000509 | 0.000256 | 0.001128 |
| Thermoclostridium stercorarium | 0.000085 | 0.00015 | 0.000197 | 0.000815 |
| Mesorhizobium sp RMAD H1 | 0.000084 | 0 | 0 | 0 |
| Pseudorhizobium pelagicum | 0.000083 | 0.001126 | 0.000582 | 0 |
| Actinophytocola algeriensis | 0.000077 | 0.000024 | 0.000149 | 0 |
| Flavobacterium subsaxonicum | 0.000076 | 0 | 0 | 0 |
| Hungateiclostridium thermocellum | 0.000075 | 0.000275 | 0.000214 | 0.003432 |
| Nocardioides sp SAI 095 | 0.000067 | 0.000029 | 0.000036 | 0 |
| Hydrogenispora sp UUS1 1 | 0.000066 | 0.000055 | 0.000191 | 0.000496 |
| Geobacillus stearothermophilus | 0.000062 | 0.000517 | 0.000288 | 0.002172 |
| Thermobifida cellulosilytica | 0.000055 | 0.000554 | 0.000293 | 0.001133 |
| Microbacterium saccharophilum | 0.000054 | 0.004776 | 0.002734 | 0 |
| Bacillus aciditolerans | 0.000051 | 0.000096 | 0.000029 | 0 |
| Glycomyces paridis | 0.00005 | 0.000153 | 0.000188 | 0 |
| Lysinibacillus endophyticus | 0.000045 | 0.00019 | 0.000099 | 0 |
| Caldicoprobacter faecalis | 0.000045 | 0.000111 | 0.000185 | 0.000359 |
| Paracoccus kondratievae | 0.000045 | 0.000495 | 0.000242 | 0.001147 |
| Symbiobacterium thermophilum | 0.000043 | 0.000086 | 0.00012 | 0.000324 |
| Thermomonospora catenispora | 0.000041 | 0.000318 | 0.000132 | 0.000229 |
| Aeribacillus pallidus | 0.000041 | 0.000161 | 0.000295 | 0.000225 |
| GGB36697 SGB52070 | 0.00004 | 0.000172 | 0.000039 | 0.000656 |
| Chelativorans sp J32 | 0.00004 | 0 | 0 | 0.000062 |
| Microbacterium sediminis | 0.000039 | 0.001981 | 0.002542 | 0.000237 |
| Actinocorallia populi | 0.000036 | 0.000021 | 0.000002 | 0.000023 |
| Thermogutta terrifontis | 0.000035 | 0.000141 | 0.000354 | 0.001385 |
| Pseudomonas sp LS 2 | 0.000035 | 0 | 0 | 0 |
| Pseudorhodoplanes sinuspersici | 0.000034 | 0 | 0 | 0 |
| Pseudomonas fluorescens | 0.000033 | 0 | 0 | 0 |
| Neorhizobium sp SOG26 | 0.000033 | 0.000661 | 0.000578 | 0.000496 |
| Pseudonocardia dioxanivorans | 0.000032 | 0 | 0 | 0 |
| Parapedobacter indicus | 0.000031 | 0.000039 | 0.000024 | 0.000017 |
| Oxalicibacterium solurbis | 0.00003 | 0.000039 | 0.000018 | 0.000047 |
| GGB38234 SGB82353 | 0.000029 | 0.00007 | 0.000077 | 0.000566 |
| Herbinix hemicellulosilytica | 0.000024 | 0.000106 | 0.00014 | 0.001027 |
| Kribbella shirazensis | 0.000023 | 0.000121 | 0.000015 | 0 |
| Jiangella endophytica | 0.000021 | 0 | 0 | 0 |
| Nocardioides sp YR527 | 0.000021 | 0.000014 | 0.000043 | 0 |
| Glycomyces harbinensis | 0.000018 | 0.000335 | 0.000082 | 0 |
| Planomicrobium glaciei | 0.000018 | 0.000278 | 0.001333 | 0 |
| Petrimonas mucosa | 0.000015 | 0.000493 | 0.000237 | 0.00943 |
| Rummeliibacillus sp POC4 | 0.000014 | 0.000133 | 0.000046 | 0.000096 |
| Pigmentiphaga litoralis | 0.000014 | 0 | 0 | 0 |
| Krasilnikoviella flava | 0.000013 | 0.000089 | 0.000078 | 0 |
| Massilia timonae | 0.000013 | 0 | 0 | 0 |
| Flavobacterium viscosus | 0.000012 | 0 | 0 | 0.000198 |
| Methanoculleus thermophilus | 0.000007 | 0.000214 | 0.000046 | 0.002426 |
| Actinocatenispora sera | 0.000004 | 0 | 0 | 0 |
| Flavobacterium macacae | 0.000004 | 0 | 0 | 0 |
| Paracoccus sp 1011MAR3C25 | 0.000002 | 0 | 0 | 0 |
| Mycolicibacterium gilvum | 0 | 0.000605 | 0.000378 | 0 |
| Xenophilus azovorans | 0 | 0 | 0 | 0 |
| Microbacterium sp Marseille Q2854 | 0 | 0 | 0 | 0.000124 |
| Pusillimonas noertemannii | 0 | 0 | 0 | 0.000051 |
| Microbacterium ginsengiterrae | 0 | 0.000706 | 0.001221 | 0.000427 |
| Parapedobacter sp SGR 10 | 0 | 0.000009 | 0 | 0.000407 |
| Methanothrix thermoacetophila | 0 | 0 | 0 | 0.01316 |
| Corynebacterium xerosis | 0 | 0 | 0 | 0.000135 |
| Dietzia kunjamensis | 0 | 0.000155 | 0.000513 | 0.004306 |
| GGB38234 SGB82354 | 0 | 0 | 0 | 0.000008 |
| Nocardia paucivorans | 0 | 0 | 0 | 0.000032 |
| Brachybacterium aquaticum | 0 | 0 | 0 | 0.000003 |
| Brachybacterium ginsengisoli | 0 | 0 | 0.000061 | 0 |
| Brachybacterium phenoliresistens | 0 | 0 | 0.000006 | 0 |
| Brachybacterium sp SGAir0954 | 0 | 0 | 0.000159 | 0 |
| GGB44037 SGB61413 | 0 | 0.002838 | 0.000478 | 0.004036 |
| Janibacter anophelis | 0 | 0 | 0 | 0.000144 |
| Janibacter indicus | 0 | 0 | 0 | 0.000121 |
| Janibacter limosus | 0 | 0 | 0.000053 | 0 |
| Flavimobilis marinus | 0 | 0.000208 | 0.000034 | 0 |
| Flavimobilis soli | 0 | 0.000137 | 0 | 0.001522 |
| Jonesia denitrificans | 0 | 0 | 0 | 0.000942 |
| Agrococcus baldri | 0 | 0.000003 | 0.000126 | 0 |
| Agrococcus jenensis | 0 | 0 | 0.000013 | 0 |
| Agrococcus lahaulensis | 0 | 0.002501 | 0.002522 | 0 |
| Curtobacterium flaccumfaciens | 0 | 0 | 0.002699 | 0 |
| Leucobacter denitrificans | 0 | 0 | 0 | 0.00011 |
| Leucobacter sp 4J7B1 | 0 | 0 | 0.000207 | 0 |
| Microbacterium algeriense | 0 | 0 | 0.000019 | 0 |
| Microbacterium hydrocarbonoxydans | 0 | 0 | 0.000179 | 0 |
| Microbacterium nanhaiense | 0 | 0 | 0 | 0.000154 |
| Microbacterium oryzae | 0 | 0 | 0.000004 | 0 |
| Microbacterium sp MEC084 | 0 | 0 | 0 | 0.000067 |
| Protaetiibacter sp SSC 01 | 0 | 0 | 0.000011 | 0.000119 |
| Salinibacterium sp dk5596 | 0 | 0.000477 | 0.000085 | 0.000113 |
| Arthrobacter sp JUb115 | 0 | 0 | 0.000189 | 0 |
| Arthrobacter sp UCD GKA | 0 | 0.000028 | 0.000322 | 0 |
| Citricoccus muralis | 0 | 0 | 0.000349 | 0 |
| Glutamicibacter arilaitensis | 0 | 0.002175 | 0.006032 | 0.004891 |
| Glutamicibacter mishrai | 0 | 0.000129 | 0.000201 | 0 |
| Glutamicibacter nicotianae | 0 | 0 | 0 | 0.00209 |
| Kocuria rosea | 0 | 0.00191 | 0.002808 | 0.000331 |
| Micrococcus endophyticus | 0 | 0 | 0 | 0.00001 |
| Micrococcus terreus | 0 | 0 | 0 | 0.000173 |
| Ornithinimicrobium flavum | 0 | 0 | 0 | 0.000012 |
| Ornithinimicrobium sp CNJ 824 | 0 | 0 | 0 | 0.000216 |
| Antribacter gilvus | 0 | 0.000062 | 0.000105 | 0 |
| Isoptericola variabilis | 0 | 0.002035 | 0.002524 | 0.000907 |
| Promicromonospora sukumoe | 0 | 0.000094 | 0.000008 | 0 |
| Micromonospora echinofusca | 0 | 0.000265 | 0 | 0 |
| Micromonospora sp HM5 17 | 0 | 0 | 0 | 0.000017 |
| GGB36012 SGB49924 | 0 | 0 | 0 | 0.00025 |
| GGB42884 SGB60103 | 0 | 0 | 0 | 0.00099 |
| GGB45191 SGB62707 | 0 | 0 | 0 | 0.000212 |
| Nocardioides cavernae | 0 | 0.00004 | 0 | 0 |
| Nocardioides daedukensis | 0 | 0.001451 | 0.001938 | 0.000016 |
| Nocardioides deserti | 0 | 0 | 0.000027 | 0 |
| Nocardioides glacieisoli | 0 | 0.00003 | 0.000012 | 0 |
| Nocardioides massiliensis | 0 | 0 | 0 | 0.000098 |
| Nocardioides seonyuensis | 0 | 0.00013 | 0.000029 | 0 |
| Nocardioides sp Root122 | 0 | 0.000734 | 0.000394 | 0 |
| Nocardioides sp SLBN 16 | 0 | 0.000025 | 0.000081 | 0 |
| Nocardioides sp Y6 | 0 | 0 | 0 | 0.000058 |
| Nocardioides szechwanensis | 0 | 0.000013 | 0 | 0 |
| Propioniciclava sp MC1595 | 0 | 0 | 0 | 0.000031 |
| Tessaracoccus flavus | 0 | 0 | 0 | 0.000018 |
| Tessaracoccus oleiagri | 0 | 0 | 0 | 0.000097 |
| Tessaracoccus rhinocerotis | 0 | 0.000572 | 0.000539 | 0.000441 |
| Saccharomonospora viridis | 0 | 0.000018 | 0 | 0 |
| GGB8530 SGB13265 | 0 | 0.000201 | 0 | 0.002469 |
| Streptomyces albidoflavus | 0 | 0 | 0.000009 | 0 |
| Streptomyces albus | 0 | 0.000109 | 0.000065 | 0 |
| Streptomyces anulatus | 0 | 0.000151 | 0.000261 | 0 |
| Streptomyces sp LamerLS 316 | 0 | 0.000084 | 0.000618 | 0 |
| Actinomadura rubrobrunea | 0 | 0 | 0 | 0.000059 |
| Thermostaphylospora chromogena | 0 | 0.000019 | 0 | 0 |
| GGB41261 SGB64010 | 0 | 0 | 0 | 0.000902 |
| Thermobaculum terrenum | 0 | 0 | 0 | 0.000372 |
| GGB23756 SGB35463 | 0 | 0 | 0 | 0.00017 |
| Proteiniphilum saccharofermentans | 0 | 0 | 0 | 0.000297 |
| Macellibacteroides fermentans | 0 | 0 | 0 | 0.000102 |
| Rhodothermus marinus | 0 | 0.000341 | 0.000159 | 0.013748 |
| Flavisolibacter sp BT320 | 0 | 0.000156 | 0.000025 | 0 |
| Dyadobacter bucti | 0 | 0.000016 | 0 | 0 |
| Leadbetterella byssophila | 0 | 0 | 0 | 0.000805 |
| Adhaeribacter aerolatus | 0 | 0.000047 | 0.000011 | 0 |
| Adhaeribacter sp DK36 | 0 | 0.000011 | 0 | 0 |
| Pontibacter russatus | 0 | 0.000161 | 0.000163 | 0 |
| Avrilella dinanensis | 0 | 0 | 0 | 0.0001 |
| Flavobacteriaceae bacterium 3519 10 | 0 | 0.000021 | 0.000135 | 0 |
| Flavobacterium cucumis | 0 | 0 | 0 | 0.000064 |
| Flavobacterium flevense | 0 | 0.00009 | 0.000031 | 0 |
| Flavobacterium haoranii | 0 | 0 | 0 | 0.000007 |
| Flavobacterium marinum | 0 | 0 | 0 | 0.000126 |
| Flavobacterium suncheonense | 0 | 0 | 0 | 0.003157 |
| Flavobacterium tibetense | 0 | 0 | 0.000031 | 0.003635 |
| Chryseobacterium sp | 0 | 0.000256 | 0.000453 | 0.000599 |
| Chryseobacterium sp cx 624 | 0 | 0.000818 | 0.002927 | 0 |
| GGB56699 SGB78132 | 0 | 0 | 0 | 0.003929 |
| Planobacterium sp GCR5 | 0 | 0 | 0 | 0.003483 |
| Geofilum rhodophaeum | 0 | 0 | 0 | 0.000759 |
| Thermophagus xiamenensis | 0 | 0 | 0 | 0.000012 |
| GGB35116 SGB79359 | 0 | 0 | 0 | 0.000116 |
| GGB23723 SGB35427 | 0 | 0 | 0 | 0.004612 |
| GGB23906 SGB35647 | 0 | 0 | 0 | 0.000126 |
| Pedobacter indicus | 0 | 0 | 0 | 0.000004 |
| Sphingobacterium alimentarium | 0 | 0.000488 | 0.000184 | 0.00283 |
| Sphingobacterium alkalisoli | 0 | 0.000065 | 0 | 0 |
| Sphingobacterium composti Yoo et al | 0 | 0.000028 | 0.000105 | 0.000706 |
| 2007 non Ten et al 2007 | | | | |
| Sphingobacterium daejeonense | 0 | 0 | 0.000044 | 0.001774 |
| Sphingobacterium endophyticum | 0 | 0 | 0 | 0.000326 |
| Sphingobacterium hotanense | 0 | 0 | 0 | 0.003154 |
| Sphingobacterium humi | 0 | 0 | 0 | 0.000136 |
| Sphingobacterium sp DN04309 | 0 | 0.000163 | 0.000238 | 0.00657 |
| Sphingobacterium sp SGG 5 | 0 | 0 | 0 | 0.000046 |
| Sphingobacterium sp T2 | 0 | 0 | 0 | 0.017846 |
| Candidatus Syntrophosphaera | 0 | 0 | 0 | 0.000615 |
| thermopropionivorans | | | | |
| Sphaerobacter SGB90588 | 0 | 0.000124 | 0.000112 | 0.000445 |
| GGB10794 SGB17457 | 0 | 0.000069 | 0.001767 | 0.003713 |
| Coprothermobacter proteolyticus | 0 | 0.000038 | 0.000445 | 0.002837 |
| GGB37021 SGB49017 | 0 | 0 | 0 | 0.001187 |
| Prochlorococcus SGB47114 | 0 | 0 | 0 | 0.000123 |
| Prochlorococcus SGB48767 | 0 | 0 | 0 | 0.000337 |
| Thermus composti | 0 | 0 | 0 | 0.000129 |
| Thermus thermophilus | 0 | 0 | 0.004971 | 0.008851 |
| Dictyoglomus thermophilum | 0 | 0 | 0.000291 | 0.005775 |
| Alicyclobacillus herbarius | 0 | 0 | 0 | 0.00009 |
| Bacillus dakarensis | 0 | 0 | 0 | 0.000102 |
| Bacillus ginsengihumi | 0 | 0.000113 | 0.000055 | 0.000189 |
| Bacillus smithii | 0 | 0 | 0.000197 | 0.000129 |
| Calditerricola satsumensis | 0 | 0.000054 | 0.000057 | 0.000214 |
| Cytobacillus oceanisediminis | 0 | 0 | 0 | 0.000201 |
| Geobacillus thermoleovorans | 0 | 0.000053 | 0 | 0.000396 |
| Lysinibacillus composti | 0 | 0.000011 | 0 | 0 |
| Metabacillus halosaccharovorans | 0 | 0 | 0 | 0.000004 |
| Metabacillus niabensis | 0 | 0.000031 | 0.000012 | 0 |
| Neobacillus thermocopriae | 0 | 0.000036 | 0.00002 | 0.000874 |
| Parageobacillus thermantarcticus | 0 | 0.000102 | 0.000099 | 0.000202 |
| Exiguobacterium mexicanum | 0 | 0 | 0 | 0.001103 |
| Brevibacillus sp SCSIO 07484 | 0 | 0 | 0 | 0.000047 |
| Cohnella laeviribosi | 0 | 0 | 0 | 0.000012 |
| Cohnella thermotolerans | 0 | 0 | 0 | 0.000028 |
| Paenibacillus antri | 0 | 0 | 0 | 0.000007 |
| Paenibacillus sp 32O W | 0 | 0 | 0 | 0.000009 |
| Planococcus massiliensis | 0 | 0.000031 | 0.000428 | 0 |
| Planococcus sp Sa1BUA13 | 0 | 0.000423 | 0.000821 | 0 |
| Planomicrobium koreense | 0 | 0.000046 | 0.000035 | 0 |
| Planomicrobium okeanokoites | 0 | 0.000077 | 0.000182 | 0 |
| Tetzosporium hominis | 0 | 0 | 0 | 0.001143 |
| Ureibacillus thermophilus | 0 | 0.000017 | 0 | 0.000092 |
| Ureibacillus thermosphaericus | 0 | 0 | 0 | 0.000209 |
| Caenibacillus caldisaponilyticus | 0 | 0.000073 | 0.000027 | 0.000276 |
| Novibacillus thermophilus | 0 | 0 | 0.000031 | 0 |
| Thermoactinomyces vulgaris | 0 | 0.000076 | 0.000149 | 0.000093 |
| Fonticella tunisiensis | 0 | 0 | 0 | 0.000103 |
| Thermaerobacter marianensis | 0 | 0 | 0 | 0.000076 |
| GGB47098 SGB65024 | 0 | 0 | 0 | 0.000281 |
| GGB58098 SGB79735 | 0 | 0 | 0 | 0.000745 |
| Hungateiclostridium clariflavum | 0 | 0 | 0 | 0.000316 |
| Pseudoclostridium thermosuccinogenes | 0 | 0 | 0 | 0.000112 |
| Thermoclostridium caenicola | 0 | 0 | 0 | 0.002831 |
| Desulfotomaculum ferrireducens | 0 | 0 | 0.000023 | 0.000003 |
| Pelotomaculum thermopropionicum | 0 | 0 | 0 | 0.000035 |
| Holdemanella SGB47126 | 0 | 0 | 0 | 0.000036 |
| Globicatella sanguinis | 0 | 0 | 0 | 0.000211 |
| GGB38793 SGB48050 | 0 | 0.000097 | 0 | 0.00471 |
| GGB42406 SGB59577 | 0 | 0 | 0 | 0.000651 |
| GGB42420 SGB59592 | 0 | 0 | 0 | 0.010486 |
| GGB42226 SGB59383 | 0 | 0 | 0 | 0.000636 |
| GGB42235 SGB59393 | 0 | 0 | 0 | 0.00009 |
| GGB42410 SGB59581 | 0 | 0 | 0 | 0.000343 |
| GGB43745 SGB61090 | 0 | 0 | 0 | 0.000148 |
| GGB44214 SGB61614 | 0 | 0 | 0 | 0.000008 |
| GGB16822 SGB82363 | 0 | 0 | 0 | 0.00005 |
| GGB42170 SGB59318 | 0 | 0 | 0 | 0.009777 |
| GGB24947 SGB36860 | 0 | 0 | 0 | 0.000658 |
| GGB25648 SGB37619 | 0 | 0 | 0.000031 | 0.000204 |
| GGB25648 SGB83014 | 0 | 0 | 0 | 0.000344 |
| GGB26746 SGB38931 | 0 | 0 | 0 | 0.001089 |
| GGB26780 SGB38965 | 0 | 0 | 0 | 0.000112 |
| GGB27142 SGB39359 | 0 | 0 | 0 | 0.000567 |
| GGB27161 SGB39379 | 0 | 0 | 0 | 0.000317 |
| GGB27163 SGB39381 | 0 | 0 | 0 | 0.000004 |
| GGB58122 SGB79760 | 0 | 0.000018 | 0.000086 | 0.001123 |
| Caldanaerobius polysaccharolyticus | 0 | 0 | 0 | 0.000052 |
| Thermacetogenium phaeum | 0 | 0.000026 | 0.000024 | 0.001051 |
| Thermanaeromonas sp C210 | 0 | 0 | 0 | 0.000005 |
| Thermoanaerobacterium | 0 | 0 | 0 | 0.000111 |
| thermosaccharolyticum | | | | |
| GGB27082 SGB39291 | 0 | 0 | 0 | 0.00017 |
| Alishewanella agri | 0 | 0 | 0 | 0.000042 |
| Bradymonadales bacterium V1718 | 0 | 0 | 0 | 0.000125 |
| Bordetella sp 02P26C 1 | 0 | 0.000015 | 0.000031 | 0.000809 |
| Paracandidimonas soli | 0 | 0 | 0 | 0.000038 |
| Pusillimonas sp 7 48 | 0 | 0 | 0 | 0.000089 |
| Pusillimonas sp NJUB218 | 0 | 0 | 0 | 0.000084 |
| Burkholderiaceae bacterium PBA | 0 | 0 | 0 | 0.000711 |
| Comamonas jiangduensis | 0 | 0 | 0 | 0.001039 |
| Comamonas kerstersii | 0 | 0 | 0 | 0.002631 |
| Pseudorhodoferax soli | 0 | 0.000009 | 0 | 0 |
| Ramlibacter sp H242 | 0 | 0.000024 | 0 | 0 |
| Schlegelella thermodepolymerans | 0 | 0.000113 | 0 | 0.007758 |
| Noviherbaspirillum soli | 0 | 0.000019 | 0.000003 | 0 |
| Brevundimonas bullata | 0 | 0.000113 | 0 | 0 |
| GGB26258 SGB38289 | 0 | 0 | 0 | 0.001119 |
| Phenylobacterium zucineum | 0 | 0 | 0 | 0.000338 |
| Cellvibrio sp PSBB006 | 0 | 0.000358 | 0.000018 | 0.000033 |
| Saccharophagus sp K07 | 0 | 0 | 0 | 0.000086 |
| Pantoea agglomerans | 0 | 0 | 0.000055 | 0 |
| Pantoea brenneri | 0 | 0 | 0.000102 | 0 |
| Chimaeribacter coloradensis | 0 | 0 | 0.000027 | 0 |
| Hydrogenophilus thermoluteolus | 0 | 0 | 0 | 0.001131 |
| Methylophilus sp Leaf414 | 0 | 0.000036 | 0 | 0 |
| GGB58125 SGB79763 | 0 | 0 | 0 | 0.001195 |
| GGB25803 SGB37788 | 0 | 0.000007 | 0 | 0.000015 |
| GGB2413 SGB3303 | 0 | 0 | 0 | 0.000167 |
| Alcanivorax pacificus | 0 | 0 | 0 | 0.002433 |
| Acinetobacter gandensis | 0 | 0 | 0 | 0.000346 |
| Acinetobacter indicus | 0 | 0 | 0 | 0.014022 |
| Acinetobacter lwoffii | 0 | 0 | 0.000031 | 0.000989 |
| Acinetobacter schindleri | 0 | 0 | 0 | 0.000414 |
| Acinetobacter sp DSM 11652 | 0 | 0 | 0 | 0.000047 |
| Acinetobacter sp HR7 | 0 | 0 | 0 | 0.005082 |
| Acinetobacter variabilis | 0 | 0.000114 | 0.000005 | 0.024821 |
| Psychrobacter alimentarius | 0 | 0 | 0.000321 | 0 |
| Pseudomonas flexibilis | 0 | 0 | 0 | 0.001571 |
| Pseudomonas formosensis | 0 | 0.001217 | 0.000474 | 0.038949 |
| Pseudomonas kunmingensis | 0 | 0.001099 | 0.000267 | 0 |
| Pseudomonas sp PDM04 | 0 | 0 | 0 | 0 |
| Pseudomonas thermotolerans | 0 | 0.000452 | 0.000129 | 0.018085 |
| Brucella intermedia | 0 | 0 | 0 | 0.000134 |
| Chelatococcus reniformis | 0 | 0 | 0 | 0 |
| Devosia submarina | 0 | 0.000143 | 0.000104 | 0 |
| Agrobacterium salinitolerans | 0 | 0.002122 | 0 | 0 |
| Rhizobium sp YS 1r | 0 | 0 | 0.000015 | 0 |
| Rhodoligotrophos appendicifer | 0 | 0 | 0 | 0 |
| Starkeya sp 3C | 0 | 0.000189 | 0.000125 | 0 |
| Gemmobacter intermedius | 0 | 0 | 0 | 0.002572 |
| Gemmobacter nectariphilus | 0 | 0 | 0 | 0.000006 |
| Paracoccus haeundaensis | 0 | 0.000477 | 0.0003 | 0 |
| Paracoccus pantotrophus | 0 | 0 | 0.000055 | 0.000692 |
| Paracoccus sp J39 | 0 | 0.000423 | 0.000233 | 0.001773 |
| Paracoccus yeei | 0 | 0.000524 | 0.000427 | 0 |
| Rhodobacter sp SGA 6 6 | 0 | 0 | 0 | 0.000004 |
| Caldovatus sediminis | 0 | 0.00001 | 0 | 0 |
| Skermanella aerolata | 0 | 0.000066 | 0.000006 | 0 |
| Rickettsia rickettsii | 0 | 0 | 0.000049 | 0 |
| Altererythrobacter sp TH136 | 0 | 0.001596 | 0.000835 | 0 |
| Croceicoccus pelagius | 0 | 0 | 0 | 0.000217 |
| Novosphingobium sp PhB55 | 0 | 0.000068 | 0 | 0 |
| Sphingobium jiangsuense | 0 | 0 | 0 | 0.000124 |
| Sphingobium sp KCTC 72723 | 0 | 0.000003 | 0 | 0 |
| Sphingobium sp MI1205 | 0 | 0.000105 | 0.00001 | 0 |
| Sphingobium sp RAC03 | 0 | 0.000151 | 0.000121 | 0 |
| Sphingomonas kaistensis | 0 | 0.000015 | 0 | 0 |
| Arenimonas sp 3729k | 0 | 0.000044 | 0 | 0.00128 |
| Lysobacter arseniciresistens | 0 | 0.000038 | 0 | 0 |
| Pseudoxanthomonas taiwanensis | 0 | 0.001117 | 0.000747 | 0.058889 |
| GGB41779 SGB58871 | 0 | 0 | 0 | 0.000399 |
| Spirochaeta thermophila | 0 | 0 | 0.000073 | 0.014753 |
| Treponema sp J25 | 0 | 0 | 0 | 0.000684 |
| GGB35627 SGB48802 | 0 | 0 | 0 | 0.002601 |
| Acetomicrobium flavidum | 0 | 0 | 0 | 0.00112 |
| Acetomicrobium hydrogeniformans | 0 | 0 | 0 | 0.000019 |
| Acholeplasma brassicae | 0 | 0 | 0 | 0.000044 |
| Acholeplasma equifetale | 0 | 0 | 0 | 0.001371 |
| Acholeplasma laidlawii | 0 | 0 | 0 | 0.000366 |
| Acholeplasma oculi | 0 | 0 | 0 | 0.002588 |
| GGB16312 SGB24698 | 0 | 0 | 0 | 0.000926 |
| Pseudothermotoga elfii | 0 | 0 | 0 | 0.002728 |
| Thermotoga caldifontis | 0 | 0 | 0 | 0.000261 |
| Thermotoga maritima | 0 | 0 | 0 | 0.000431 |
| Thermotoga profunda | 0 | 0 | 0 | 0.002157 |

### Example 2: Distribution of Bacterial Consortium in Liquid Culture and Biofilm Over Time

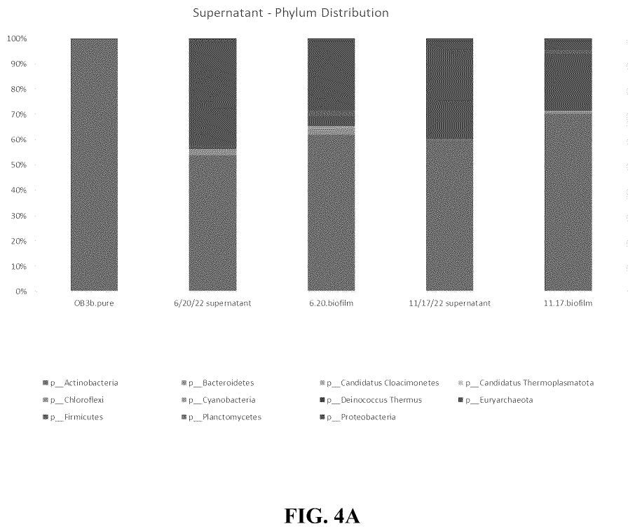

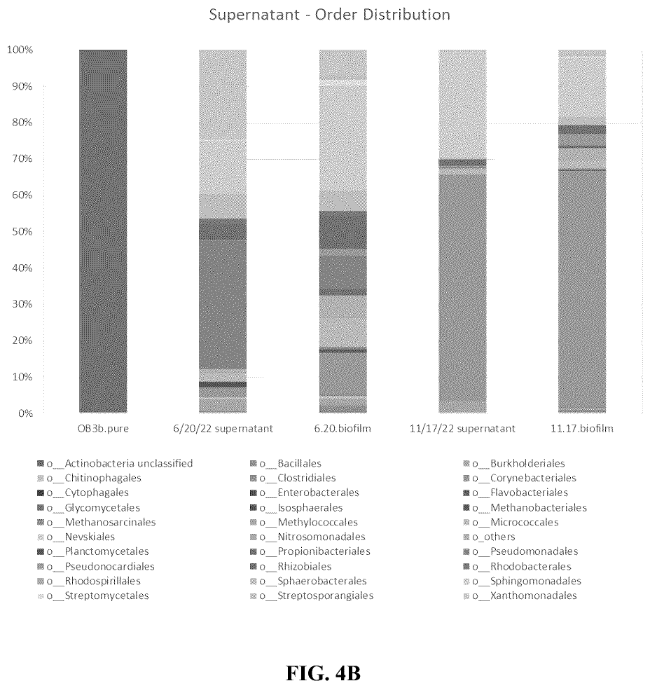

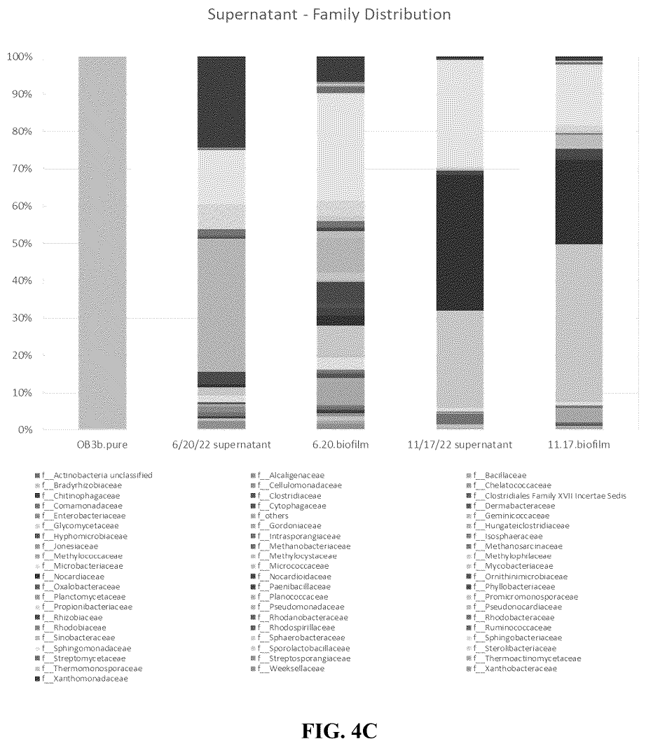

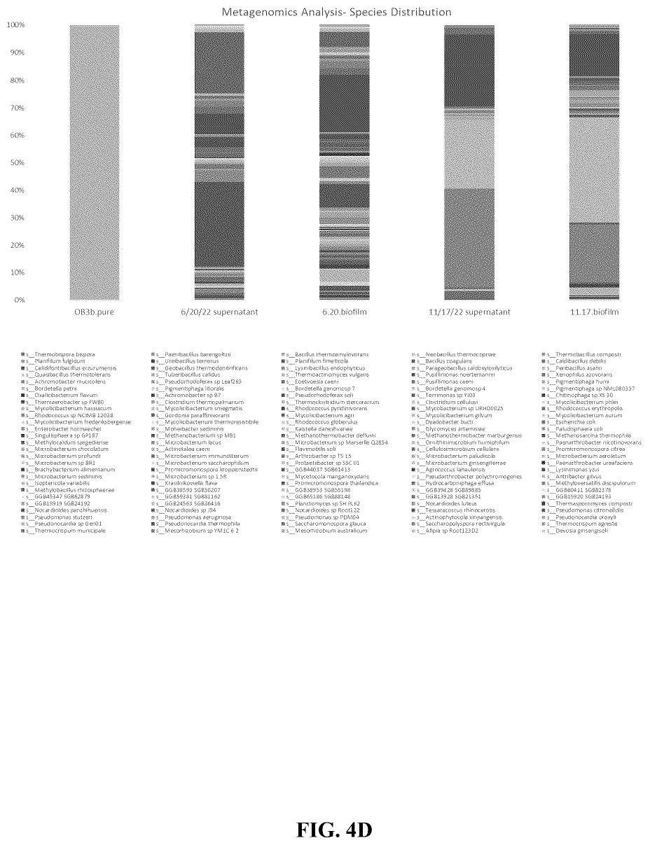

To determine the species distribution of the bacterial consortium in liquid culture over time, metagenomic analysis of a liquid culture was performed at various time points (FIGS. 4A-4D). To begin the culture (timepoint 0, “OB3b.pure”), 200 ml of bacterial culture consisted of approximately 10 g compost, 20 g Methylosinus trichosporium OB3b culture, and 180 g modified Nitrate Mineral Salts (NMS) media prepared with nitrate omitted. The modified NMS medium consisted of 1 g/L magnesium sulfate heptahydrate (MgSO₄·7H₂O), 0.2 g/L calcium chloride dihydrate (CaCl₂·2H₂O), 0.11 g/L monopotassium phosphate (KH₂PO₄), 0.18 g/L dibasic potassium phosphate (K₂HPO₄), 1 ml of ethylenediaminetetraacetic acid ferric sodium salt (FeNaEDTA) stock solution (1000×), and 1 ml of trace metal stock solution (1000×). The trace metal stock solution (1000×) consisted of 0.04 g/L sodium molybdate dihydrate (Na₂MoO₄·2H₂O), 0.2 g/L copper(II) sulfate pentahydrate (CuSO₄·5H₂O), 0.4 g/L zinc sulfate heptahydrate (ZnSO₄·7H₂O), 0.02 g/L manganese(II) chloride tetrahydrate (MnCl₂·4H₂O), 0.05 g/L cobalt(II) chloride hexahydrate (CoCl₂·6H₂O), 0.01 g/L nickel(II) chloride hexahydrate (NiCl₂·6H₂O), 0.015 g/L boric acid (H₃BO₃), and 0.25 g/L ethylenediaminetetraacetic acid (EDTA) in deionized water. The FeNaEDTA stock solution (1000×) was made of 0.0065 g/L FeNaEDTA in deionized water. The pH of modified NMS medium is around 7.

The OB3b culture, compost, and media was placed into a 500 mL glass jar covered with loose lid. The culture was grown under ambient outdoor conditions in an enclosed environment connected to natural gas line maintaining approximately 1000-8000 ppm methane in air. The culture was aerated by continuous bubbling and maintained in the dark.

Samples consisting of 2 mL liquid culture were collected at three time points: time 0, time 1 (6/20/22; 4 months after inoculation), and time 2 (11/27/22; 8 months after inoculation) (FIGS. 4A-4D). For comparison, the bacterial composition of the compost control that was not inoculated with the bacterial consortium was sequenced in Example 1 (FIG. 3).

The culture was measured at three timepoints: time 0 (03/2022), time 1 (6/20/2022), and time 2 (11/27/2022). At each time point, the sample was frozen and sent to Zymo Research on dry ice using overnight shipping. At time point 1 and 2, biofilm was also harvested from the culture and 0.5 mL of the biofilm was sent to Zymo Research on dry ice using overnight shipping. The culture and biofilm samples were processed and analyzed with the ZymoBIOMICS® Shotgun Metagenomic Sequencing Service for Microbiome Analysis as described above.

The Phylum distribution of the bacterial consortium in liquid culture and biofilm changed over time (Table 6, FIG. 4A).

TABLE 6
Phylum distribution of liquid culture and biofilm over time

| | | Jun. 20, 2022 | Jun. 20, 2022 | Nov. 17, 2022 | Nov. 17, 2022 |
|---|---|---|---|---|---|
| Phylum | OB3b.pure | supernatant | biofilm | supernatant | biofilm |
| Actinobacteria | 1 | 0.537019 | 0.619585 | 0.601505 | 0.702312 |
| Bacteroidetes | 0 | 0.02326 | 0.027917 | 0.001371 | 0.008682 |
| Candidatus Cloacimonetes | 0 | 0 | 0.000488 | 0 | 0.000544 |
| Candidatus | 0 | 0.001383 | 0.001451 | 0 | 0 |
| Thermoplasmatota | | | | | |
| Chloroflexi | 0 | 0.002 | 0.004597 | 0 | 0.002157 |
| Cyanobacteria | 0 | 0 | 0.000689 | 0 | 0.000289 |
| Deinococcus Thermus | 0 | 0 | 0.000676 | 0 | 0.000192 |
| Euryarchaeota | 0 | 0.008007 | 0.03725 | 0.362556 | 0.224813 |
| Firmicutes | 0 | 0.00406 | 0.023253 | 0.000123 | 0.011907 |
| Planctomycetes | 0 | 0 | 0.000529 | 0 | 0.000371 |
| Proteobacteria | 0 | 0.424274 | 0.28356 | 0.034444 | 0.048745 |

The order distribution of the bacterial consortium in liquid culture and biofilm changed over time (Table 7, FIG. 4B3).

TABLE 7
Order distribution of liquid culture and biofilm over time

| | | Jun. 20, 2022 | Jun. 20, 2022 | Nov. 17, 2022 | Nov. 17, 2022 |
|---|---|---|---|---|---|
| Order | OB3b.pure | supernatant | biofilm | supernatant | biofilm |
| Actinobacteria unclassified | 0 | 0.002326 | 0.004222 | 0.000123 | 0.001626 |
| Bacillales | 0 | 0.004006 | 0.017139 | 0.000118 | 0.008204 |
| Burkholderiales | 0 | 0.032497 | 0.018042 | 0.032991 | 0.003982 |
| Chitinophagales | 0 | 0.004404 | 0.008504 | 0 | 0.001288 |
| Clostridiales | 0 | 0.000168 | 0.003623 | 0.000192 | 0.002991 |
| Corynebacteriales | 0 | 0.02799 | 0.11469 | 0.623712 | 0.647877 |
| Cytophagales | 0 | 0.000013 | 0.000132 | 0 | 0 |
| Enterobacterales | 0 | 0.012937 | 0.002677 | 0 | 0 |
| Flavobacteriales | 0 | 0.002561 | 0.002977 | 0 | 0 |
| Glycomycetales | 0 | 0 | 0.000371 | 0 | 0.000067 |
| Isosphaerales | 0 | 0 | 0.000045 | 0 | 0 |
| Methanobacteriales | 0 | 0 | 0.001941 | 0 | 0.001976 |
| Methanosarcinales | 0 | 0 | 0.007017 | 0 | 0.004262 |
| Methylococcales | 0 | 0.000011 | 0.001186 | 0 | 0.000486 |
| Micrococcales | 0 | 0.025057 | 0.079566 | 0.01166 | 0.021326 |
| Nevskiales | 0 | 0.000049 | 0.000051 | 0 | 0 |
| Nitrosomonadales | 0 | 0.001455 | 0.000857 | 0 | 0 |
| -Others | 0 | 0.007235 | 0.061382 | 0.005097 | 0.034909 |
| Planctomycetales | 0 | 0 | 0.000114 | 0 | 0.000076 |
| Propionibacteriales | 0 | 0.00047 | 0.015989 | 0.00217 | 0.007059 |
| Pseudomonadales | 0 | 0.353841 | 0.092455 | 0 | 0 |
| Pseudonocardiales | 0 | 0.001147 | 0.018835 | 0.004554 | 0.0328 |
| Rhizobiales | 1 | 0.043325 | 0.090017 | 0.017777 | 0.02167 |
| Rhodobacterales | 0 | 0.015855 | 0.014419 | 0 | 0.002879 |
| Rhodospirillales | 0 | 0.000449 | 0.000164 | 0 | 0.000015 |
| Sphaerobacterales | 0 | 0.066738 | 0.055152 | 0.005023 | 0.02141 |
| Sphingomonadales | 0 | 0.146896 | 0.288904 | 0.287087 | 0.161832 |
| Streptomycetales | 0 | 0.003175 | 0.017266 | 0.001408 | 0.005555 |
| Streptosporangiales | 0 | 0 | 0.010377 | 0 | 0.006061 |
| Xanthomonadales | 0 | 0.247398 | 0.071881 | 0.008087 | 0.011661 |

The family distribution of the bacterial consortium in liquid culture and biofilm changed over time (Table 8, FIG. 4C).

TABLE 8
Family distribution of liquid culture and biofilm over time

| | | Jun. 20, 2022 | Jun. 20, 2022 | Nov. 17, 2022 | Nov. 17, 2022 |
|---|---|---|---|---|---|
| Family | OB3b.pure | supernatant | biofilm | supernatant | biofilm |
| Actinobacteria unclassified | 0 | 0.002326 | 0.004222 | 0.000123 | 0.001626 |
| Alcaligenaceae | 0 | 0.020702 | 0.011207 | 0.0047 | 0.001659 |
| Bacillaceae | 0 | 0.000731 | 0.008155 | 0 | 0.004176 |
| Bradyrhizobiaceae | 0 | 0.005743 | 0.01281 | 0.008938 | 0.002041 |
| Cellulomonadaceae | 0 | 0.001093 | 0.007687 | 0 | 0.001333 |
| Chelatococcaceae | 0 | 0 | 0.000346 | 0 | 0 |
| Chitinophagaceae | 0 | 0.004404 | 0.008504 | 0 | 0.001288 |
| Clostridiaceae | 0 | 0 | 0.000366 | 0 | 0.000269 |
| Clostridiales Family XVII | 0 | 0.000168 | 0.003201 | 0.000192 | 0.002533 |
| Incertae Sedis | | | | | |
| Comamonadaceae | 0 | 0.011795 | 0.006835 | 0.028131 | 0.002323 |
| Cytophagaceae | 0 | 0.000013 | 0.000132 | 0 | 0 |
| Dermabacteraceae | 0 | 0 | 0 | 0.000234 | 0.001358 |
| Enterobacteriaceae | 0 | 0.014671 | 0.004314 | 0 | 0.000064 |
| f_others | 0 | 0.007235 | 0.061382 | 0.005097 | 0.034909 |
| Geminicoccaceae | 0 | 0 | 0.00003 | 0 | 0 |
| Glycomycetaceae | 0 | 0 | 0.000371 | 0 | 0.000067 |
| Gordoniaceae | 0 | 0.000413 | 0.00881 | 0.000594 | 0.005088 |
| Hungateiclostridiaceae | 0 | 0 | 0 | 0 | 0.000013 |
| Hyphomicrobiaceae | 0 | 0.00444 | 0.012116 | 0.000545 | 0.000365 |
| Intrasporangiaceae | 0 | 0 | 0.000745 | 0 | 0.000543 |
| Isosphaeraceae | 0 | 0 | 0.000045 | 0 | 0 |
| Jonesiaceae | 0 | 0.000957 | 0.000089 | 0 | 0 |
| Methanobacteriaceae | 0 | 0 | 0.001941 | 0 | 0.001976 |
| Methanosarcinaceae | 0 | 0 | 0.007017 | 0 | 0.004262 |
| Methylococcaceae | 0 | 0.000011 | 0.001186 | 0 | 0.000486 |
| Methylocystaceae | 1 | 0.000006 | 0.000169 | 0 | 0 |
| Methylophilaceae | 0 | 0.000614 | 0.000461 | 0 | 0 |
| Microbacteriaceae | 0 | 0.016654 | 0.031964 | 0.01033 | 0.009847 |
| Micrococcaceae | 0 | 0.002447 | 0.005711 | 0.000432 | 0.003155 |
| Mycobacteriaceae | 0 | 0.020291 | 0.079514 | 0.258918 | 0.417548 |
| Nocardiaceae | 0 | 0.007286 | 0.026366 | 0.3642 | 0.225241 |
| Nocardioidaceae | 0 | 0.00047 | 0.020522 | 0.00217 | 0.009751 |
| Ornithinimicrobiaceae | 0 | 0.002522 | 0.012443 | 0.000151 | 0.000104 |
| Oxalobacteraceae | 0 | 0 | 0 | 0.00016 | 0 |
| Paenibacillaceae | 0 | 0.002905 | 0.00161 | 0 | 0.000276 |
| Phyllobacteriaceae | 0 | 0.027182 | 0.055662 | 0.00822 | 0.019136 |
| Planctomycetaceae | 0 | 0 | 0.000114 | 0 | 0.000076 |
| Planococcaceae | 0 | 0.000055 | 0.004107 | 0.000118 | 0.001875 |
| Promicromonosporaceae | 0 | 0.001384 | 0.020927 | 0.000513 | 0.004986 |
| Propionibacteriaceae | 0 | 0 | 0.000194 | 0 | 0 |
| Pseudomonadaceae | 0 | 0.353841 | 0.092455 | 0 | 0 |
| Pseudonocardiaceae | 0 | 0.001147 | 0.018835 | 0.004554 | 0.0328 |
| Rhizobiaceae | 0 | 0.004858 | 0.007229 | 0 | 0.000065 |
| Rhodanobacteraceae | 0 | 0.003407 | 0.003456 | 0.000304 | 0.000182 |
| Rhodobacteraceae | 0 | 0.015855 | 0.014419 | 0 | 0.002879 |
| Rhodobiaceae | 0 | 0.000372 | 0.000286 | 0 | 0.000063 |
| Rhodospirillaceae | 0 | 0.000449 | 0.000134 | 0 | 0.000015 |
| Ruminococcaceae | 0 | 0 | 0.000056 | 0 | 0.000176 |
| Sinobacteraceae | 0 | 0.000049 | 0.000051 | 0 | 0 |
| Sphaerobacteraceae | 0 | 0 | 0.013995 | 0.000121 | 0.005885 |
| Sphingobacteriaceae | 0 | 0.066738 | 0.041157 | 0.004902 | 0.015525 |
| Sphingomonadaceae | 0 | 0.145162 | 0.287267 | 0.287087 | 0.161768 |
| Sporolactobacilluseae | 0 | 0 | 0.000717 | 0 | 0.000304 |
| Sterolibacteriaceae | 0 | 0.000841 | 0.000396 | 0 | 0 |
| Streptomycetaceae | 0 | 0.003175 | 0.017266 | 0.001408 | 0.005555 |
| Streptosporangiaceae | 0 | 0 | 0.000886 | 0 | 0.000538 |
| Thermoactinomycetaceae | 0 | 0.000315 | 0.00255 | 0 | 0.001573 |
| Thermomonosporaceae | 0 | 0 | 0.004764 | 0 | 0.002831 |
| Weeksellaceae | 0 | 0.002561 | 0.002977 | 0 | 0 |
| Xanthobacteraceae | 0 | 0.000724 | 0.001399 | 0.000074 | 0 |
| Xanthomonadaceae | 0 | 0.243991 | 0.068425 | 0.007783 | 0.011479 |

The genus distribution of the bacterial consortium in liquid culture and biofilm changed over time (Table 9, FIG. 4D).

TABLE 9
Genus distribution of liquid culture and biofilm over time

| | | Jun. 20, 2022 | Jun. 20, 2022 | Nov. 17, 2022 | Nov. 17, 2022 |
|---|---|---|---|---|---|
| Genus | OB3b.pure | supernatant | biofilm | supernatant | biofilm |
| Achromobacter | 0 | 0.005676 | 0.00278 | 0 | 0.000106 |
| Actinophytocola | 0 | 0.000766 | 0.008215 | 0.001795 | 0.00397 |
| Actinotalea | 0 | 0.001093 | 0.007687 | 0 | 0.001333 |
| Afipia | 0 | 0.005294 | 0.009484 | 0 | 0.000584 |
| Agrococcus | 0 | 0 | 0.000312 | 0 | 0 |
| Albibacterium | 0 | 0.020622 | 0.011625 | 0 | 0.000012 |
| Aminobacter | 0 | 0 | 0.000495 | 0 | 0.000018 |
| Antribacter | 0 | 0 | 0.000013 | 0 | 0 |
| Aquamicrobium | 0 | 0 | 0 | 0 | 0.000012 |
| Arthrobacter | 0 | 0.000271 | 0.000562 | 0 | 0 |
| Azospirillum | 0 | 0.000449 | 0.000134 | 0 | 0.000015 |
| Bacillus | 0 | 0.000378 | 0.002623 | 0 | 0.001573 |
| Bordetella | 0 | 0.000345 | 0.000114 | 0 | 0 |
| Bosea | 0 | 0.000449 | 0.003326 | 0 | 0.000171 |
| Brachybacterium | 0 | 0 | 0 | 0.000234 | 0.001358 |
| Caldibacillus | 0 | 0 | 0.000317 | 0 | 0.000149 |
| Calidifontibacillus | 0 | 0 | 0.000032 | 0 | 0.000003 |
| Cellulosimicrobium | 0 | 0.000791 | 0.00981 | 0 | 0.001675 |
| Cereibacter | 0 | 0 | 0.000051 | 0 | 0 |
| Chelatococcus | 0 | 0 | 0.000346 | 0 | 0 |
| Chitinophaga | 0 | 0.000131 | 0.000101 | 0 | 0 |
| Clostridium | 0 | 0 | 0.000366 | 0 | 0.000269 |
| Devosia | 0 | 0.004166 | 0.011616 | 0 | 0.000237 |
| Dokdonella | 0 | 0.000853 | 0.000656 | 0.000304 | 0.000182 |
| Dyadobacter | 0 | 0.000013 | 0.000132 | 0 | 0 |
| Enterobacter | 0 | 0.000026 | 0 | 0 | 0 |
| Eoetvoesia | 0 | 0.00143 | 0.002349 | 0 | 0.000073 |
| Erythrobacter | 0 | 0.001734 | 0.001637 | 0 | 0.000064 |
| Escherichia | 0 | 0.012911 | 0.002677 | 0 | 0 |
| Flavimobilis | 0 | 0.000957 | 0.000089 | 0 | 0 |
| Geminicoccus | 0 | 0 | 0.00003 | 0 | 0 |
| Geobacillus | 0 | 0 | 0.004467 | 0 | 0.002076 |
| g_OTHERS | 0 | 0.007235 | 0.062127 | 0.005097 | 0.035452 |
| Glycomyces | 0 | 0 | 0.000371 | 0 | 0.000067 |
| Gordonia | 0 | 0.000413 | 0.00881 | 0.000594 | 0.005088 |
| Hydrocarboniphaga | 0 | 0.000049 | 0.000051 | 0 | 0 |
| Hyphomicrobium | 0 | 0.000274 | 0.0005 | 0.000545 | 0.000128 |
| Isoptericola | 0 | 0 | 0.001114 | 0 | 0 |
| Kaistella | 0 | 0.00035 | 0.001481 | 0 | 0 |
| Kaistia | 0 | 0 | 0.000007 | 0 | 0 |
| Krasilnikoviella | 0 | 0 | 0.000072 | 0 | 0.000009 |
| Luteimonas | 0 | 0.000564 | 0.00092 | 0.005615 | 0.01005 |
| Lysinibacillus | 0 | 0 | 0 | 0 | 0.000004 |
| Lysinimonas | 0 | 0 | 0.000034 | 0 | 0 |
| Mesorhizobium | 0 | 0.027138 | 0.054629 | 0.007211 | 0.015 |
| Methanobacterium | 0 | 0 | 0.000079 | 0 | 0.000243 |
| Methanosarcina | 0 | 0 | 0.007017 | 0 | 0.004262 |
| Methanothermobacter | 0 | 0 | 0.001862 | 0 | 0.001733 |
| Methylobacillus | 0 | 0.000614 | 0.000461 | 0 | 0 |
| Methylocaldum | 0 | 0.000011 | 0.001186 | 0 | 0.000486 |
| Methylosinus | 1 | 0.000006 | 0.000169 | 0 | 0 |
| Methyloversatilis | 0 | 0.000841 | 0.000396 | 0 | 0 |
| Microbacterium | 0 | 0.01664 | 0.031259 | 0.01033 | 0.009815 |
| Moheibacter | 0 | 0.002211 | 0.001496 | 0 | 0 |
| Mycetocola | 0 | 0 | 0.000332 | 0 | 0.000032 |
| Mycobacterium | 0 | 0.003023 | 0.013733 | 0.000457 | 0.005651 |
| Mycolicibacterium | 0 | 0.017268 | 0.065781 | 0.258461 | 0.411897 |
| Neobacillus | 0 | 0.000353 | 0.000067 | 0 | 0 |
| Neorhizobium | 0 | 0.00002 | 0.000194 | 0 | 0 |
| Nitratireductor | 0 | 0.000044 | 0.000538 | 0 | 0.000768 |
| Nocardioides | 0 | 0.000276 | 0.013967 | 0.002119 | 0.005778 |
| Novosphingobium | 0 | 0.000075 | 0.000007 | 0 | 0 |
| Oligotropha | 0 | 0 | 0 | 0.008938 | 0.001286 |
| Olivibacter | 0 | 0.001373 | 0.00127 | 0.000299 | 0.000027 |
| Ornithinimicrobium | 0 | 0.002522 | 0.012443 | 0.000151 | 0.000104 |
| Oxalicibacterium | 0 | 0 | 0 | 0.00016 | 0 |
| Paenarthrobacter | 0 | 0.002176 | 0.00495 | 0.000432 | 0.003155 |
| Paenibacillus | 0 | 0.002555 | 0.000823 | 0 | 0.000038 |
| Paludisphaera | 0 | 0 | 0.000006 | 0 | 0 |
| Paracoccus | 0 | 0.015855 | 0.014368 | 0 | 0.002879 |
| Parageobacillus | 0 | 0 | 0.00012 | 0 | 0 |
| Parapedobacter | 0 | 0.043264 | 0.02765 | 0.004603 | 0.015486 |
| Peribacillus | 0 | 0 | 0.000367 | 0 | 0.000211 |
| Pigmentiphaga | 0 | 0.000531 | 0.000792 | 0.000832 | 0.001191 |
| Planctomyces | 0 | 0 | 0.000114 | 0 | 0.000076 |
| Planifilum | 0 | 0.000315 | 0.002532 | 0 | 0.001573 |
| Promicromonospora | 0 | 0.000593 | 0.009918 | 0.000513 | 0.003302 |
| Protaetiibacter | 0 | 0.000014 | 0.000027 | 0 | 0 |
| Pseudaminobacter | 0 | 0 | 0.000199 | 0.001009 | 0.003338 |
| Pseudolabrys | 0 | 0 | 0 | 0.000063 | 0 |
| Pseudomonas | 0 | 0.353841 | 0.092455 | 0 | 0 |
| Pseudonocardia | 0 | 0.000381 | 0.004326 | 0.002759 | 0.023505 |
| Pseudorhizobium | 0 | 0 | 0.001344 | 0 | 0.000065 |
| Pseudorhodoferax | 0 | 0.003935 | 0.003826 | 0 | 0.000243 |
| Pseudoxanthomonas | 0 | 0.241938 | 0.065462 | 0 | 0.001376 |
| Pusillimonas | 0 | 0.01272 | 0.005172 | 0.003868 | 0.000289 |
| Quasibacillus | 0 | 0 | 0.000162 | 0 | 0.00016 |
| Rhodanobacter | 0 | 0.002554 | 0.0028 | 0 | 0 |
| Rhodococcus | 0 | 0.007286 | 0.026366 | 0.3642 | 0.225241 |
| Rhodoligotrophos | 0 | 0.000372 | 0.000286 | 0 | 0.000063 |
| Ruminococcaceae unclassified | 0 | 0 | 0.000056 | 0 | 0.000176 |
| Saccharomonospora | 0 | 0 | 0.001302 | 0 | 0.000896 |
| Saccharopolyspora | 0 | 0 | 0.002301 | 0 | 0.001728 |
| Shinella | 0 | 0.004838 | 0.005684 | 0 | 0 |
| Singulisphaera | 0 | 0 | 0.000039 | 0 | 0 |
| Sphaerobacter | 0 | 0 | 0.013995 | 0.000121 | 0.005885 |
| Sphingobacterium | 0 | 0.001479 | 0.000612 | 0 | 0 |
| Sphingobium | 0 | 0.040904 | 0.043233 | 0.024894 | 0.008679 |
| Sphingopyxis | 0 | 0.104183 | 0.244027 | 0.262193 | 0.153089 |
| Starkeya | 0 | 0.000724 | 0.001399 | 0.000011 | 0 |
| Stenotrophomonas | 0 | 0.000028 | 0.000465 | 0.002168 | 0.000053 |
| Streptomyces | 0 | 0.003175 | 0.017266 | 0.001408 | 0.005555 |
| Terrimonas | 0 | 0.004273 | 0.008403 | 0 | 0.001288 |
| Tessaracoccus | 0 | 0 | 0.000194 | 0 | 0 |
| Thermaerobacter | 0 | 0.000168 | 0.003201 | 0.000192 | 0.002533 |
| Thermasporomyces | 0 | 0.000194 | 0.001846 | 0.000051 | 0.001281 |
| Thermobacillus | 0 | 0.00035 | 0.000787 | 0 | 0.000238 |
| Thermobifida | 0 | 0 | 0.004727 | 0 | 0.002692 |
| Thermobispora | 0 | 0.002326 | 0.004222 | 0.000123 | 0.001626 |
| Thermoclostridium | 0 | 0 | 0 | 0 | 0.000013 |
| Thermocrispum | 0 | 0 | 0.002691 | 0 | 0.002701 |
| Thermomonas | 0 | 0.001461 | 0.001578 | 0 | 0 |
| Thermomonospora | 0 | 0 | 0 | 0 | 0.004764 |
| Thermopolyspora | 0 | 0 | 0.000886 | 0 | 0.000538 |
| Tuberibacillus | 0 | 0 | 0.000717 | 0 | 0.000304 |
| Ureibacillus | 0 | 0.000055 | 0.004107 | 0.000118 | 0.001875 |
| Xenophilus | 0 | 0.00786 | 0.003009 | 0.028131 | 0.00208 |

The species distribution of the bacterial consortium in liquid culture and biofilm changed over time (Table 10, FIG. 4D).

TABLE 10
Species distribution of liquid culture and biofilm over time

| | | Jun. 20, 2022 | Jun. 20, 2022 | Nov. 17, 2022 | Nov. 17, 2022 |
|---|---|---|---|---|---|
| Species | OB3b.pure | supernatant | biofilm | supernatant | biofilm |
| Thermobispora bispora | 0 | 0.002326 | 0.004222 | 0.000123 | 0.001626 |
| Paenibacillus barengoltzii | 0 | 0.002555 | 0.000823 | 0 | 0.000038 |
| Bacillus thermoamylovorans | 0 | 0.000378 | 0.001352 | 0 | 0.000488 |
| Neobacillus thermocopriae | 0 | 0.000353 | 0.000067 | 0 | 0 |
| Thermobacillus composti | 0 | 0.00035 | 0.000787 | 0 | 0.000238 |
| Planifilum fulgidum | 0 | 0.000269 | 0.001955 | 0 | 0.001374 |
| Ureibacillus terrenus | 0 | 0.000055 | 0.004107 | 0.000118 | 0.001875 |
| Planifilum fimeticola | 0 | 0.000046 | 0.000577 | 0 | 0.000199 |
| Bacillus coagulans | 0 | 0 | 0.001271 | 0 | 0.001085 |
| Caldibacillus debilis | 0 | 0 | 0.000317 | 0 | 0.000149 |
| Calidifontibacillus erzurumensis | 0 | 0 | 0.000032 | 0 | 0.000003 |
| Geobacillus thermodenitrificans | 0 | 0 | 0.004467 | 0 | 0.002076 |
| Lysinibacillus endophyticus | 0 | 0 | 0 | 0 | 0.000004 |
| Parageobacillus | 0 | 0 | 0.00012 | 0 | 0 |
| caldoxylosilyticus | | | | | |
| Peribacillus asahii | 0 | 0 | 0.000367 | 0 | 0.000211 |
| Quasibacillus thermotolerans | 0 | 0 | 0.000162 | 0 | 0.00016 |
| Tuberibacillus calidus | 0 | 0 | 0.000717 | 0 | 0.000304 |
| Thermoactinomyces vulgaris | 0 | 0 | 0.000018 | 0 | 0 |
| Pusillimonas noertemannii | 0 | 0.011314 | 0.004837 | 0.003868 | 0.000289 |
| Xenophilus azovorans | 0 | 0.00786 | 0.003009 | 0.028131 | 0.00208 |
| Achromobacter mucicolens | 0 | 0.005676 | 0.001353 | 0 | 0 |
| Pseudorhodoferax sp Leaf265 | 0 | 0.003935 | 0.002759 | 0 | 0 |
| Eoetvoesia caeni | 0 | 0.00143 | 0.002349 | 0 | 0.000073 |
| Pusillimonas caeni | 0 | 0.001406 | 0.000335 | 0 | 0 |
| Pigmentiphaga humi | 0 | 0.000503 | 0.000134 | 0 | 0 |
| Bordetella petrii | 0 | 0.000331 | 0.000114 | 0 | 0 |
| Pigmentiphaga litoralis | 0 | 0.000028 | 0.000658 | 0 | 0.00002 |
| Bordetella genomosp 7 | 0 | 0.000011 | 0 | 0 | 0 |
| Bordetella genomosp 4 | 0 | 0.000003 | 0 | 0 | 0 |
| Pigmentiphaga sp NML080357 | 0 | 0 | 0 | 0.000832 | 0.001171 |
| Oxalicibacterium flavum | 0 | 0 | 0 | 0.00016 | 0 |
| Achromobacter sp B7 | 0 | 0 | 0.001427 | 0 | 0.000106 |
| Pseudorhodoferax soli | 0 | 0 | 0.001067 | 0 | 0.000243 |
| Terrimonas sp YJ03 | 0 | 0.004273 | 0.008403 | 0 | 0.001288 |
| Chitinophaga sp XS 30 | 0 | 0.000131 | 0.000101 | 0 | 0 |
| Thermaerobacter sp FW80 | 0 | 0.000168 | 0.003201 | 0.000192 | 0.002533 |
| Clostridium thermopalmarium | 0 | 0 | 0.000366 | 0 | 0.000269 |
| Thermoclostridium stercorarium | 0 | 0 | 0 | 0 | 0.000013 |
| Clostridium cellulosi | 0 | 0 | 0.000056 | 0 | 0.000176 |
| Mycolicibacterium phlei | 0 | 0.006739 | 0.014308 | 0.001488 | 0.002402 |
| Mycolicibacterium hassiacum | 0 | 0.005141 | 0.041368 | 0.004532 | 0.023466 |
| Mycolicibacterium smegmatis | 0 | 0.005088 | 0.008699 | 0.001338 | 0.001234 |
| Rhodococcus pyridinivorans | 0 | 0.004446 | 0.013987 | 0.002113 | 0.010804 |
| Mycobacterium sp URHD0025 | 0 | 0.003023 | 0.013733 | 0.000457 | 0.005651 |
| Rhodococcus erythropolis | 0 | 0.001495 | 0.005647 | 0.001617 | 0.00384 |
| Rhodococcus sp NCIMB 12038 | 0 | 0.001345 | 0.00639 | 0.36047 | 0.210492 |
| Gordonia paraffinivorans | 0 | 0.000413 | 0.00881 | 0.000594 | 0.005088 |
| Mycolicibacterium agri | 0 | 0.000179 | 0.000755 | 0 | 0.000134 |
| Mycolicibacterium gilvum | 0 | 0.000121 | 0.000605 | 0.251103 | 0.384568 |
| Mycolicibacterium aurum | 0 | 0 | 0 | 0 | 0.000002 |
| Mycolicibacterium | 0 | 0 | 0 | 0 | 0.00008 |
| frederiksbergense | | | | | |
| Mycolicibacterium | 0 | 0 | 0.000046 | 0 | 0.000011 |
| thermoresistibile | | | | | |
| Rhodococcus globerulus | 0 | 0 | 0.000342 | 0 | 0.000105 |
| Dyadobacter bucti | 0 | 0.000013 | 0.000132 | 0 | 0 |
| Escherichia coli | 0 | 0.012911 | 0.002677 | 0 | 0 |
| Enterobacter hormaechei | 0 | 0.000026 | 0 | 0 | 0 |
| Moheibacter sediminis | 0 | 0.002211 | 0.001496 | 0 | 0 |
| Kaistella daneshvariae | 0 | 0.00035 | 0.001481 | 0 | 0 |
| Glycomyces artemisiae | 0 | 0 | 0.000371 | 0 | 0.000067 |
| Paludisphaera soli | 0 | 0 | 0.000006 | 0 | 0 |
| Singulisphaera sp GP 187 | 0 | 0 | 0.000039 | 0 | 0 |
| Methanobacterium sp MB1 | 0 | 0 | 0.000079 | 0 | 0.000243 |
| Methanothermobacter defluvii | 0 | 0 | 0.000765 | 0 | 0.000673 |
| Methanothermobacter | 0 | 0 | 0.001097 | 0 | 0.00106 |
| marburgensis | | | | | |
| Methanosarcina thermophila | 0 | 0 | 0.007017 | 0 | 0.004262 |
| Methylocaldum szegediense | 0 | 0.000011 | 0.001186 | 0 | 0.000486 |
| Microbacterium lacus | 0 | 0.009127 | 0.012756 | 0 | 0.001292 |
| Microbacterium sp Marseille | 0 | 0.004639 | 0.0083 | 0.007518 | 0.003571 |
| Q2854 | | | | | |
| Ornithinimicrobium humiphilum | 0 | 0.002522 | 0.012443 | 0.000151 | 0.000104 |
| Paenarthrobacter | 0 | 0.002176 | 0.00495 | 0 | 0.000794 |
| nicotinovorans | | | | | |
| Microbacterium chocolatum | 0 | 0.001725 | 0.002826 | 0.000214 | 0.001781 |
| Actinotalea caeni | 0 | 0.001093 | 0.007687 | 0 | 0.001333 |
| Flavimobilis soli | 0 | 0.000957 | 0.000089 | 0 | 0 |
| Cellulosimicrobium cellulans | 0 | 0.000791 | 0.00981 | 0 | 0.001675 |
| Promicromonospora citrea | 0 | 0.000593 | 0.006537 | 0.000447 | 0.001985 |
| Microbacterium profundi | 0 | 0.000463 | 0.001431 | 0 | 0.000022 |
| Microbacterium immunditiarum | 0 | 0.000427 | 0.001577 | 0.000048 | 0.001438 |
| Arthrobacter sp TS 15 | 0 | 0.000271 | 0.000562 | 0 | 0 |
| Microbacterium paludicola | 0 | 0.000101 | 0.001137 | 0 | 0.000135 |
| Microbacterium aerolatum | 0 | 0.00008 | 0.001127 | 0 | 0.000154 |
| Microbacterium sp BR1 | 0 | 0.000047 | 0.0001 | 0 | 0 |
| Microbacterium saccharophilum | 0 | 0.000031 | 0.000893 | 0 | 0.000194 |
| Protaetiibacter sp SSC 01 | 0 | 0.000014 | 0.000027 | 0 | 0 |
| Microbacterium ginsengiterrae | 0 | 0 | 0.000092 | 0.00255 | 0.001228 |
| Paenarthrobacter ureafaciens | 0 | 0 | 0 | 0.000432 | 0.002361 |
| Brachybacterium alimentarium | 0 | 0 | 0 | 0.000234 | 0.001358 |
| Promicromonospora | 0 | 0 | 0.003118 | 0.000066 | 0.001317 |
| kroppenstedtii | | | | | |
| GGB44037 SGB61413 | 0 | 0 | 0.000745 | 0 | 0.000543 |
| Agrococcus lahaulensis | 0 | 0 | 0.000312 | 0 | 0 |
| Lysinimonas yzui | 0 | 0 | 0.000034 | 0 | 0 |
| Microbacterium sediminis | 0 | 0 | 0.000513 | 0 | 0 |
| Microbacterium sp 1 5R | 0 | 0 | 0.000507 | 0 | 0 |
| Mycetocola manganoxydans | 0 | 0 | 0.000332 | 0 | 0.000032 |
| Pseudarthrobacter | 0 | 0 | 0.000199 | 0 | 0 |
| polychromogenes | | | | | |
| Antribacter gilvus | 0 | 0 | 0.000013 | 0 | 0 |
| Isoptericola variabilis | 0 | 0 | 0.001114 | 0 | 0 |
| Krasilnikoviella flava | 0 | 0 | 0.000072 | 0 | 0.000009 |
| Promicromonospora thailandica | 0 | 0 | 0.000263 | 0 | 0 |
| Hydrocarboniphaga effusa | 0 | 0.000049 | 0.000051 | 0 | 0 |
| Methyloversatilis discipulorum | 0 | 0.000841 | 0.000396 | 0 | 0 |
| Methylobacillus rhizosphaerae | 0 | 0.000614 | 0.000461 | 0 | 0 |
| GGB38593 SGB50207 | 0 | 0 | 0.002449 | 0 | 0.002768 |
| GGB38953 SGB55198 | 0 | 0.00031 | 0.004009 | 0.000055 | 0.001687 |
| GGB39428 SGB89885 | 0 | 0.002844 | 0.001387 | 0 | 0 |
| GGB60411 SGB82378 | 0 | 0.000033 | 0 | 0 | 0 |
| GGB45347 SGB62879 | 0 | 0.000011 | 0.001588 | 0 | 0 |
| GGB59381 SGB81162 | 0 | 0.000295 | 0.000144 | 0 | 0.001446 |
| GGB65186 SGB88148 | 0 | 0.000054 | 0.003712 | 0.000086 | 0.00114 |
| GGB13928 SGB21351 | 0 | 0 | 0.000224 | 0 | 0 |
| GGB15920 SGB24193 | 0 | 0.002026 | 0.024608 | 0.002345 | 0.014651 |
| GGB15919 SGB24192 | 0 | 0.001033 | 0.018871 | 0.001787 | 0.012961 |
| GGB24563 SGB36416 | 0 | 0.000629 | 0.00439 | 0.000824 | 0.000256 |
| Planctomyces sp SH PL62 | 0 | 0 | 0.000114 | 0 | 0.000076 |
| Nocardioides luteus | 0 | 0.000276 | 0.013177 | 0.002119 | 0.005664 |
| Thermasporomyces composti | 0 | 0.000194 | 0.001828 | 0.000051 | 0.001281 |
| Nocardioides panzhihuensis | 0 | 0 | 0.000157 | 0 | 0.000114 |
| Nocardioides sp J54 | 0 | 0 | 0.000558 | 0 | 0 |
| Nocardioides sp Root122 | 0 | 0 | 0.000075 | 0 | 0 |
| Tessaracoccus rhinocerotis | 0 | 0 | 0.000194 | 0 | 0 |
| Pseudomonas citronellolis | 0 | 0.30769 | 0.080091 | 0 | 0 |
| Pseudomonas stutzeri | 0 | 0.042434 | 0.011636 | 0 | 0 |
| Pseudomonas aeruginosa | 0 | 0.003713 | 0.000728 | 0 | 0 |
| Pseudomonas sp PDM04 | 0 | 0.000004 | 0 | 0 | 0 |
| Actinophytocola xinjiangensis | 0 | 0.000766 | 0.008215 | 0.001795 | 0.00397 |
| Pseudonocardia oroxyli | 0 | 0.000381 | 0.003377 | 0.002759 | 0.02273 |
| Pseudonocardia sp Gen01 | 0 | 0 | 0.000037 | 0 | 0.000153 |
| Pseudonocardia thermophila | 0 | 0 | 0.000912 | 0 | 0.000622 |
| Saccharomonospora glauca | 0 | 0 | 0.001302 | 0 | 0.000896 |
| Saccharopolyspora rectivirgula | 0 | 0 | 0.002301 | 0 | 0.001728 |
| Thermocrispum agreste | 0 | 0 | 0.000488 | 0 | 0.000544 |
| Thermocrispum municipale | 0 | 0 | 0.002203 | 0 | 0.002157 |
| Mesorhizobium sp YM1C 6 2 | 0 | 0.018619 | 0.033914 | 0.005127 | 0.012826 |
| Mesorhizobium australicum | 0 | 0.007803 | 0.013004 | 0.002084 | 0.002082 |
| Afipia sp Root123D2 | 0 | 0.005294 | 0.009484 | 0 | 0.000584 |
| Devosia ginsengisoli | 0 | 0.004087 | 0.010377 | 0 | 0.000046 |
| Shinella granuli | 0 | 0.002704 | 0.003961 | 0 | 0 |
| Shinella fusca | 0 | 0.001158 | 0.000499 | 0 | 0 |
| Shinella zoogloeoides | 0 | 0.000976 | 0.001224 | 0 | 0 |
| Mesorhizobium sp DCY119 | 0 | 0.000716 | 0.006694 | 0 | 0.000092 |
| Starkeya novella | 0 | 0.00061 | 0.001246 | 0.000011 | 0 |
| Bosea robiniae | 0 | 0.000449 | 0.003326 | 0 | 0.000171 |
| Rhodoligotrophos appendicifer | 0 | 0.000372 | 0.000286 | 0 | 0.000063 |
| Hyphomicrobium sp | 0 | 0.000274 | 0.0005 | 0.000545 | 0.000128 |
| NDB2Meth4 | | | | | |
| Starkeya sp OAE902 | 0 | 0.000114 | 0.000153 | 0 | 0 |
| Devosia limi | 0 | 0.000048 | 0.000648 | 0 | 0.00017 |
| Nitratireductor aestuarii | 0 | 0.000044 | 0.000538 | 0 | 0.000768 |
| Devosia elaeis | 0 | 0.000031 | 0.000063 | 0 | 0 |
| Neorhizobium sp SOG26 | 0 | 0.00002 | 0.000194 | 0 | 0 |
| Methylosinus trichosporium | 1 | 0.000006 | 0.000169 | 0 | 0 |
| Oligotropha carboxidovorans | 0 | 0 | 0 | 0.008938 | 0.001286 |
| Pseudaminobacter | 0 | 0 | 0 | 0.001009 | 0.003338 |
| salicylatoxidans | | | | | |
| Pseudolabrys taiwanensis | 0 | 0 | 0 | 0.000063 | 0 |
| Chelatococcus composti | 0 | 0 | 0.000273 | 0 | 0 |
| Chelatococcus reniformis | 0 | 0 | 0.000073 | 0 | 0 |
| Devosia naphthalenivorans | 0 | 0 | 0.000528 | 0 | 0.000021 |
| Aminobacter sp J41 | 0 | 0 | 0.000495 | 0 | 0.000018 |
| Aquamicrobium defluvii | 0 | 0 | 0 | 0 | 0.000012 |
| Mesorhizobium sp J18 | 0 | 0 | 0.000003 | 0 | 0 |
| Mesorhizobium sp Root552 | 0 | 0 | 0.000048 | 0 | 0 |
| Mesorhizobium zhangyense | 0 | 0 | 0.000966 | 0 | 0 |
| Kaistia adipata | 0 | 0 | 0.000007 | 0 | 0 |
| Pseudorhizobium banfieldiae | 0 | 0 | 0.001344 | 0 | 0.000065 |
| Paracoccus sp J39 | 0 | 0.013654 | 0.012437 | 0 | 0.001932 |
| Paracoccus versutus | 0 | 0.00141 | 0.001241 | 0 | 0.000947 |
| Paracoccus pantotrophus | 0 | 0.000791 | 0.000548 | 0 | 0 |
| Cereibacter changlensis | 0 | 0 | 0.000051 | 0 | 0 |
| Paracoccus yeei | 0 | 0 | 0.000142 | 0 | 0 |
| Azospirillum oryzae | 0 | 0.000419 | 0.000134 | 0 | 0.000015 |
| Azospirillum thiophilum | 0 | 0.00003 | 0 | 0 | 0 |
| Geminicoccus sp CPCC 101082 | 0 | 0 | 0.00003 | 0 | 0 |
| Sphaerobacter thermophilus | 0 | 0 | 0.013995 | 0.000121 | 0.005885 |
| Parapedobacter pyrenivorans | 0 | 0.020778 | 0.013818 | 0 | 0 |
| Albibacterium bauzanense | 0 | 0.020622 | 0.011625 | 0 | 0.000012 |
| Parapedobacter indicus | 0 | 0.015627 | 0.008362 | 0.00042 | 0.006656 |
| Parapedobacter defluvii | 0 | 0.003533 | 0.002534 | 0.003336 | 0.007282 |
| Parapedobacter sp SGR 10 | 0 | 0.003326 | 0.002936 | 0.000847 | 0.001269 |
| Olivibacter jilunii | 0 | 0.001373 | 0.00127 | 0.000299 | 0.000027 |
| Sphingobacterium sp SGG 5 | 0 | 0.000854 | 0.000364 | 0 | 0 |
| Sphingobacterium olei | 0 | 0.000625 | 0.000248 | 0 | 0 |
| Parapedobacter luteus | 0 | 0 | 0 | 0 | 0.000279 |
| Sphingopyxis terrae | 0 | 0.075647 | 0.207868 | 0.261756 | 0.152051 |
| Sphingobium cupriresistens | 0 | 0.024937 | 0.023511 | 0.024894 | 0.008568 |
| Sphingopyxis sp OPL5 | 0 | 0.020319 | 0.025149 | 0 | 0.000029 |
| Sphingobium sp RAC03 | 0 | 0.010307 | 0.009848 | 0 | 0 |
| Sphingopyxis sp GW 247 27LB | 0 | 0.004645 | 0.006224 | 0 | 0 |
| Sphingobium baderi | 0 | 0.004153 | 0.004121 | 0 | 0 |
| Erythrobacter sp SG61 1L | 0 | 0.001734 | 0.001637 | 0 | 0.000064 |
| Sphingopyxis fribergensis | 0 | 0.001725 | 0.002652 | 0 | 0.000032 |
| Sphingobium limneticum | 0 | 0.001507 | 0.005753 | 0 | 0 |
| Sphingopyxis sp 113P3 | 0 | 0.000857 | 0.001063 | 0.000201 | 0.000844 |
| Sphingopyxis lindanitolerans | 0 | 0.000555 | 0.000468 | 0 | 0 |
| Sphingopyxis sp JAI108 | 0 | 0.000431 | 0.000592 | 0 | 0 |
| Novosphingobium sp AP12 | 0 | 0.000075 | 0 | 0 | 0 |
| Sphingopyxis macrogoltabida | 0 | 0.000004 | 0 | 0 | 0 |
| Sphingopyxis granuli | 0 | 0 | 0 | 0.000236 | 0.000133 |
| Novosphingobium resinovorum | 0 | 0 | 0.000007 | 0 | 0 |
| Sphingobium jiangsuense | 0 | 0 | 0 | 0 | 0.000111 |
| Sphingopyxis italica | 0 | 0 | 0.000011 | 0 | 0 |
| Streptomyces cellulosae | 0 | 0.00305 | 0.01334 | 0.001371 | 0.003822 |
| Streptomyces sp NBRC 109706 | 0 | 0.000125 | 0.001751 | 0.000037 | 0.000692 |
| Streptomyces mimosae | 0 | 0 | 0.000689 | 0 | 0.000289 |
| Streptomyces sp CNQ329 | 0 | 0 | 0.000676 | 0 | 0.000192 |
| Streptomyces sp SBST2 5 | 0 | 0 | 0.000683 | 0 | 0.000363 |
| Streptomyces sp SBT349 | 0 | 0 | 0.000083 | 0 | 0 |
| Streptomyces thermoviolaceus | 0 | 0 | 0.000044 | 0 | 0.000197 |
| Thermobifida cellulosilytica | 0 | 0 | 0.000007 | 0 | 0 |
| Thermobifida fusca | 0 | 0 | 0.00472 | 0 | 0.002692 |
| Thermopolyspora flexuosa | 0 | 0 | 0.000886 | 0 | 0.000538 |
| Thermomonospora curvata | 0 | 0 | 0.004764 | 0 | 0.002831 |
| Pseudoxanthomonas suwonensis | 0 | 0.220003 | 0.045706 | 0 | 0.00007 |
| Pseudoxanthomonas | 0 | 0.011197 | 0.012538 | 0 | 0 |
| yeongjuensis | | | | | |
| Pseudoxanthomonas taiwanensis | 0 | 0.004334 | 0.000621 | 0 | 0.001306 |
| Pseudoxanthomonas sp 3HH 4 | 0 | 0.002965 | 0.00273 | 0 | 0 |
| Pseudoxanthomonas mexicana | 0 | 0.002 | 0.002394 | 0 | 0 |
| Rhodanobacter sp Root480 | 0 | 0.001525 | 0.001491 | 0 | 0 |
| Pseudoxanthomonas | 0 | 0.001383 | 0.001451 | 0 | 0 |
| jiangsuensis | | | | | |
| Rhodanobacter fulvus | 0 | 0.001029 | 0.001309 | 0 | 0 |
| Thermomonas fusca | 0 | 0.000954 | 0.001159 | 0 | 0 |
| Dokdonella koreensis | 0 | 0.000853 | 0.000656 | 0.000304 | 0.000182 |
| Thermomonas sp XSG | 0 | 0.000507 | 0.000419 | 0 | 0 |
| Luteimonas lumbrici | 0 | 0.000252 | 0.000283 | 0 | 0 |
| Luteimonas sp J29 | 0 | 0.000183 | 0.000388 | 0.005615 | 0.01005 |
| Luteimonas mephitis | 0 | 0.000129 | 0.000249 | 0 | 0 |
| Pseudoxanthomonas sp PXM03 | 0 | 0.000056 | 0.000022 | 0 | 0 |
| Stenotrophomonas maltophilia | 0 | 0.000028 | 0 | 0 | 0 |
| Stenotrophomonas rhizophila | 0 | 0 | 0.000465 | 0.002168 | 0.000053 |

### Example 3: Compost Comprising the Bacterial Consortium Improves Soil Microbiome and Nitrogen Content Compared to Compost that Lacks the Bacterial Consortium

A compost pile comprising 0.5 kg of biomass was inoculated with 0.5 L of Methylosinus trichosporium OB3b culture at an optical density at 600 nm of 40. A control compost pile comprising 0.5 kg of biomass was setup without bacteria added. Both compost piles were maintained in an enclosed environment with 1000-8000 ppm of methane and ambient outdoor temperature and pressure conditions. Aliquots (l Og) of compost were extracted from each pile at different time points: 0 weeks, 2 weeks, 4 weeks, 6 weeks and 10 weeks. Each sample was freeze dried, ground to a fine powder and analyzed for carbon and nitrogen content using a Dumas combustion process. Total moisture content was analyzed using an oven drying method. Total nitrogen %, total carbon %, moisture % and total N/C ratios were calculated for each compost pile.

The results of the Dumas combustion analysis and an oven drying method are shown in Table 11.

TABLE 11
Analysis of compost samples over time with and without bacteria added.

| | | Total | Total | Total | |
|---|---|---|---|---|---|
| | Time point | Nitrogen | Carbon | Nitrogen/Carbon | |
| Samples | (week) | (%) | (%) | Ratio | Moisture (%) |
| Compost Control | 0 | 0.97 | 34.95 | 0.028 | 4.75 |
| | 2 | 1.09 | 31.91 | 0.034 | 3.59 |
| | 4 | 1.01 | 31.97 | 0.032 | 3.52 |
| | 6 | 1.07 | 31.26 | 0.034 | 2.79 |
| | 10 | 1.07 | 28.61 | 0.037 | 2.77 |
| Compost + OB3b | 0 | 1.1 | 32.74 | 0.034 | 5.81 |
| | 2 | 1.12 | 32.8 | 0.034 | 6.28 |
| | 4 | 1.15 | 28.85 | 0.040 | 4.43 |
| | 6 | 1.3 | 28.88 | 0.045 | 2.61 |
| | 10 | 1.2 | 31.07 | 0.039 | 3.2 |

As shown in Table 11, the compost sample with the addition of the bacteria had overall higher levels of total nitrogen % and total nitrogen/carbon ratio at each time point analyzed.

### Example 4: Compost Comprising the Bacterial Consortium Improves Plant Growth Compared to Compost that Lacks the Bacterial Consortium

A compost pile comprising a biomass will be inoculated with the consortium of bacteria described herein. The compost pile will be maintained in a closed outdoor environment under ambient temperature and pressure conditions. The compost will be enriched with a methane gas input. The methane will be provided to the consortium of bacteria at a concentration of about 90 ppm, about 100 ppm, about 110 ppm, about 120 ppm, about 130 ppm, about 140 ppm, about 150 ppm, about 200 ppm, about 300 ppm, about 400 ppm, about 500 ppm, about 600 ppm, about 700 ppm, about 800 ppm, about 900 ppm, about 1000 ppm, about 2000 ppm, about 3000 ppm, about 4000 ppm, about 5000 ppm, about 6000 ppm, about 7000 ppm, about 8000 ppm, about 9000 ppm, or higher for about 1 week, about 2 weeks, about 3 weeks, about 4 weeks, about 5 weeks, about 6 weeks, about 7 weeks, about 8 weeks, or longer. A similar compost pile will be treated under the same conditions and parameters, but will not be inoculated with the consortium of bacteria.

Soil from multiple geographical sites will be transferred to a greenhouse containing atmospheric methane and nitrogen concentrations with ambient temperature and pressure. Each soil sample will be divided equally into 18 pots in order to grow 3 different plants in triplicate for an untreated and a treated condition. The plants will include corn, soybeans, and wheat. Each plant will be potted in a pot comprising treated soil (soil mixed with compost inoculated with the consortium of bacteria) or untreated soil (soil mixed with compost not inoculated with the consortium of bacteria). The same amount and mass of seeds will be planted in each plot. The plant growth will be monitored for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. Each week, each pot will be examined for soil microbiome, nitrogen content of the soil, stalk length, sprout number and length, fruit or seedling amount and size, leaf and stalk color, and leaf length. Additionally, at the terminal time point, each pot will be further examined for root biomass weight, total plant weight, and microbiome associated with the roots.

### Example 5: Bacterial Consortium Reduces Methane Output of Industrial Site

A reactor comprising the bacterial consortium disclosed herein will be connected to an existing ventilation system at an industrial site. The consortium of the bacteria will be introduced into the reactor in a liquid or solid culture along with the additives as disclosed herein. The ventilation system will streamline the gas from the industrial site and deliver it to the reactor that will contain the bacterial consortium. The gas from the industrial site will contain methane to provide the carbon source for the consortium of bacteria at a concentration of about 90 ppm, about 100 ppm, about 110 ppm, about 120 ppm, about 130 ppm, about 140 ppm, about 150 ppm, about 200 ppm, about 300 ppm, about 400 ppm, about 500 ppm, about 600 ppm, about 700 ppm, about 800 ppm, about 900 ppm, about 1000 ppm, about 2000 ppm, about 3000 ppm, about 4000 ppm, about 5000 ppm, about 6000 ppm, about 7000 ppm, about 8000 ppm, about 9000 ppm, or higher. The input concentration of methane may change over the course of a day, a week, or a month.

The reactor will be maintained at ambient temperature and pressure. The input and output concentrations of methane will be measured using a gas chromatography or a portable methane detector. The concentrations of methane will be measured at various time points over the course of about 1 day, about 2 days, about 3 days, about 4 days, about 5 days, about 6 days, or about 7 days. The concentrations of methane will be measured at various time points over the course of about 1 week, about 2 weeks, about 3 weeks, about 4 weeks, about 5 weeks, about 6 weeks, about 7 weeks, about 8 weeks, or longer. The concentration of methane will show a reduction in methane of about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, or about 100% between the input concentration and output concentration of methane.

### Example 6: Bacterial Consortium Reduces Methane Output of a Farm

A reactor comprising a compost and the bacterial consortium disclosed herein will be connected to an existing ventilation system at a farm. The compost may comprise cow manure from the cows in the farm or other sources of biomass. The consortium of the bacteria will be introduced into the compost pile along with the additives as disclosed herein. The ventilation system will streamline the gas from the farm and deliver it to the reactor that will contain the compost inoculated with the bacterial consortium. The gas from the farm will contain methane to provide the carbon source for the consortium of bacteria at a concentration of about 90 ppm, about 100 ppm, about 110 ppm, about 120 ppm, about 130 ppm, about 140 ppm, about 150 ppm, about 200 ppm, about 300 ppm, about 400 ppm, about 500 ppm, about 600 ppm, about 700 ppm, about 800 ppm, about 900 ppm, about 1000 ppm, about 2000 ppm, about 3000 ppm, about 4000 ppm, about 5000 ppm, about 6000 ppm, about 7000 ppm, about 8000 ppm, about 9000 ppm, 10,000 ppm, 11,000 ppm, 12,000 ppm, 13,000 ppm, 14,000 ppm, 15,000 ppm, 16,000 ppm, 17,000 ppm, 18,000 ppm, 19,000 ppm, 20,000 ppm, 25,000 ppm, 30,000 ppm, 35,000 ppm, 40,000 ppm, 45,000 ppm, or 50,000 ppm. The input concentration of methane may change over the course of a day, a week, or a month.

The reactor will begin at ambient temperature and pressure and will experience elevated temperatures and elevated pressure. The input and output concentrations of methane will be measured using gas chromatography or a portable methane detector. The concentrations of methane will be measured at various time points over the course of about 1 day, about 2 days, about 3 days, about 4 days, about 5 days, about 6 days, about 7 days, about 1 week, about 2 weeks, about 3 weeks, about 4 weeks, about 5 weeks, about 6 weeks, about 7 weeks, about 8 weeks, or longer. The concentration of methane will show a reduction in methane of about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, or about 100% between the input concentration and output concentration of methane.

The nitrogen fixation rate will also be measured in the reactor by measuring nitrogen fixation of the compost pile over time using gas chromatography or acetylene reduction assay. The nitrogen content of the compost pile will include all nitrogen in the form of ammonia, ammonium, nitrates, nitrites, or any combination thereof. The input compost will contain about 0.1%, about 0.2%, about 0.3%, about 0.4%, about 0.5%, about 0.6%, about 0.7%, about 0.8%, about 0.9%, or about 1.0% nitrogen. The nitrogen fixation will show an increase in nitrogen content of the compost of about 100%, about 200%, about 300%, about 400%, about 500%, about 600%, about 700%, about 800%, about 900%, about 1000% between the input concentration and output concentration. The total amount of nitrogen in the output compost will be at least about 1%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, or more total nitrogen in the compost pile.

### Example 7: Bacterial Consortium Reduces Methane Output of a Dairy Barn

The bacterial consortium as described herein will be introduced into a dairy barn housing animals, for example, dairy cows. The bacterial consortium will be introduced into the housing on the soil, hay, or other housing material surrounding the animals along with the additives as disclosed herein. The bacterial consortium will fix nitrogen and consume methane from the ambient air in the barn or from reactors that concentrate the air from the barn. The air from the barn will contain methane to provide the carbon source for the consortium of bacteria at a concentration of CH₄ at atmospheric concentrations or at concentrations of at least about 90 ppm, at least about 100 ppm, at least about 110 ppm, at least about 120 ppm, at least about 130 ppm, at least about 140 ppm, at least about 150 ppm, at least about 200 ppm, at least about 250 ppm, at least about 300 ppm, at least about 400 ppm, at least about 500 ppm, at least about 600 ppm, at least about 700 ppm, at least about 800 ppm, or at least about 900 ppm. The input concentration of methane may change over the course of a day, a week, or a month. The bacterial consortium will be replenished in the dairy barn at a frequency of about once per week, once every other week, once a month, once every other month, once every three months, once every four months, once every five months, once every six months, once every eight months, once every ten months, once a year, or once every other year. The additives will be replenished in the dairy barn at a frequency of about once per week, once every other week, once a month, once every other month, once every three months, once every four months, once every five months, once every six months, once every eight months, once every ten months, once a year, or once every other year.

The dairy barn will be maintained at ambient temperature and pressure and will experience elevated temperatures and decreased temperatures. The input and output concentrations of methane will be measured using gas chromatography or a portable methane detector. The concentrations of methane will be measured at various time points over the course of about 1 day, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, or longer. The concentration of methane will show a reduction in methane of at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% relative to the concentration of methane at a similar barn that does not comprise the bacterial consortium.

### Example 8: Bacterial Consortium Reduces Methane Output of a Landfill

The bacterial consortium as described herein will be introduced onto a landfill or in or around a ventilation pipe or pipes that collect or release gas from the landfill pile into the air. The bacterial consortium will be introduced onto the landfill in cases where the landfill is open to ambient air and in cases where the landfill is covered.

### Open Landfill

In cases where the landfill is open to the ambient air, the bacterial consortium and the additives will be introduced to a landfill pile or to a ventilation pipe or pipes that collect or release gas from the landfill pile to the air in a liquid, spray, powder, gel, or matrix format. The bacterial consortium will be replenished on or in the landfill or ventilation pipe at a frequency of about once per week, once every other week, once a month, once every other month, once every three months, once every four months, once every five months, once every six months, once every eight months, once every ten months, once a year, or once every other year. The additives will be replenished on or in the landfill or ventilation pipe at a frequency of about once per week, once every other week, once a month, once every other month, once every three months, once every four months, once every five months, once every six months, once every eight months, once every ten months, once a year, or once every other year.

The bacterial consortium will fix nitrogen and consume methane from the ambient air in the landfill or from the air in the ventilation pipe. The air from the landfill or ventilation pipe will contain methane to provide the carbon source for the consortium of bacteria at a concentration of CH₄ at atmospheric concentrations or at concentrations of at least about 20 ppm, at least about 25 ppm, at least about 30 ppm, at least about 40 ppm, at least about 50 ppm, at least about 60 ppm, at least about 70 ppm, at least about 80 ppm, at least about 90 ppm, at least about 100 ppm, at least about 110 ppm, at least about 120 ppm, at least about 130 ppm, at least about 140 ppm, at least about 150 ppm, at least about 200 ppm, at least about 250 ppm, at least about 300 ppm, at least about 400 ppm, at least about 500 ppm, at least about 600 ppm, at least about 700 ppm, at least about 800 ppm, or at least about 900 ppm. The input concentration of methane in the air or ventilation pipe may change over the course of a day, a week, a month, or a year.

The landfill or ventilation pipe will be maintained at ambient temperature and pressure and will experience elevated temperatures, decreased temperatures, elevated pressure, and decreased pressure. The input and output concentrations of methane will be measured using gas chromatography or a portable methane detector. The concentrations of methane will be measured at various time points over the course of about 1 day, about 2 days, about 3 days, about 4 days, about 5 days, about 6 days, or about 7 days, about 2 weeks, about 3 weeks, about 4 weeks, about 5 weeks, about 6 weeks, about 7 weeks, about 8 weeks, or longer. The concentration of methane will show a reduction in methane of at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% between the input concentration and output concentration of methane. The concentration of methane will show a reduction in methane of at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to the concentration of methane from a landfill that does not comprise the bacterial consortium.

### Covered Landfill

The bacterial consortium will be introduced to a separate landfill pile as a cover over the landfill pile or the ventilation pipe that collects or releases gas from inside the landfill pile to the air. The bacterial consortium will be seeded in a cover or temporary cover that covers the landfill pile or the ventilation pipe, and/or introduced as a biogenic amendment to a cover or temporary cover. Additives will also be introduced into the cover or biogenic amendment. The bacterial consortium will be replenished in the cover or biogenic amendment at a frequency of about once per week, once every other week, once a month, once every other month, once every three months, once every four months, once every five months, once every six months, once every eight months, once every ten months, once a year, or once every other year. The additives will be replenished in the cover or biogenic amendment at a frequency of about once per week, once every other week, once a month, once every other month, once every three months, once every four months, once every five months, once every six months, once every eight months, once every ten months, once a year, or once every other year.

The bacterial consortium will fix nitrogen and consume methane from the ambient air in the landfill or ventilation pipe. The air from the landfill or ventilation pipe will contain methane to provide the carbon source for the consortium of bacteria at a concentration of CH₄ at atmospheric concentrations or at concentrations of at least about 20 ppm, at least about 25 ppm, at least about 30 ppm, at least about 40 ppm, at least about 50 ppm, at least about 60 ppm, at least about 70 ppm, at least about 80 ppm, at least about 90 ppm, at least about 100 ppm, at least about 110 ppm, at least about 120 ppm, at least about 130 ppm, at least about 140 ppm, at least about 150 ppm, at least about 200 ppm, at least about 250 ppm, at least about 300 ppm, at least about 400 ppm, at least about 500 ppm, at least about 600 ppm, at least about 700 ppm, at least about 800 ppm, at least about 900 ppm, or higher. The input concentration of methane in the air or ventilation pipe may change over the course of a day, a week, a month, or a year.

The landfill or ventilation pipe will be maintained at ambient temperature and pressure and will experience elevated temperatures, decreased temperatures, elevated pressure, and decreased pressure. The input and output concentrations of methane will be measured using gas chromatography or a portable methane detector. The concentrations of methane will be measured at various time points over the course of about 1 day, about 2 days, about 3 days, about 4 days, about 5 days, about 6 days, or about 7 days, about 2 weeks, about 3 weeks, about 4 weeks, about 5 weeks, about 6 weeks, about 7 weeks, about 8 weeks, or longer. The concentration of methane will show a reduction in methane of at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% between the input concentration and output concentration of methane.

### Example 9: Bacterial Consortium Reduces Methane Output and/or Nitrogen Fixation of a Rice Field

Rice fields will be inoculated with the consortium of bacteria as disclosed herein. The consortium of bacteria will be introduced to the soil, water, other medium in which the rice plants are grown or growing, on the leaves, stems, or on the roots of the plant. The bacterial consortium will be applied as a spray, a powder, or in a liquid. The consortium of bacteria will be replenished on or in the rice fields at a frequency of about once daily, once per week, once every other week, once a month, once every other month, once every three months, once every four months, once every five months, once every six months, once every eight months, once every ten months, once a year, once every other year, or at a rate that is necessary, and may change in frequency. The additives will be replenished on or in the rice field at a frequency of about once per week, once every other week, once a month, once every other month, once every three months, once every four months, once every five months, once every six months, once every eight months, once every ten months, once a year, once every other year, or at a rate that is necessary, and may change in frequency.

The rice fields will incubate with the consortium of bacteria for about 1 week, about 2 weeks, about 3 weeks, about 4 weeks, about 5 weeks, about 6 weeks, about 7 weeks, about 8 weeks, or longer. The rice fields will be maintained in an outdoor environment under ambient temperature and pressure conditions. The consortium of bacteria will use nitrogen or methane from the water, ambient air, from emissions of the plant such as from the roots, leaves, or stems, or from the methane-producing bacteria that grow in rice fields or flooded rice fields.

The nitrogen fixation rate will be measured in the rice fields by measuring nitrogen fixation of the rice fields (including in the soil, plants, air, or water) over time using acetylene reduction assay and/or gas chromatography. The nitrogen content of the soil, plants, air, or water will include all nitrogen in the form of ammonia, ammonium, nitrates, nitrites, or any combination thereof.

The bacterial consortium will consume methane from the ambient air, from the air around the rice field, from the stem, roots, and leaves of the rice plants, or from the bacteria that grow in the water and soil around the rice plants. The methane will provide the carbon source for the consortium of bacteria at a concentration of CH₄ at atmospheric concentrations or at concentrations of at least about 2 ppm, at least about 2.5 ppm, at least about 3 ppm, at least about 3.50 ppm, at least about 4 ppm, at least about 4.5 ppm, at least about 5 ppm, at least about 6 ppm, at least about 6.5 ppm, at least about 7 ppm, at least about 7.5 ppm, at least about 8 ppm, at least about 8.50 ppm, at least about 9 ppm, at least about 9.5 ppm, at least about 10 ppm, at least about 15 ppm, at least about 20 ppm, at least about 30 ppm, at least about 40 ppm, at least about 50 ppm, at least about 75 ppm, at least about 100 ppm, at least about 200 ppm, at least about 300 ppm, or higher in and around the rice fields. The concentration of methane in the air in and around rice fields may change over the course of a day, a week, a month, a year, or more.

The rice fields will be maintained at ambient temperature and pressure and will experience elevated temperatures, decreased temperatures, elevated pressure, decreased pressure, and flooding. The concentrations of methane will be measured using gas chromatography or a portable methane detector. The concentrations of methane will be measured at various time points over the course of about 1 day, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, or longer. The concentration of methane will show a reduction in methane of at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to the concentration of methane from a similar rice field that does not comprise the bacterial consortium.

### Example 10: Poorly Managed Compost Comprising the Bacterial Consortium Improves Methane Consumption and Nitrogen Content

A compost pile comprising a biomass will be inoculated with the consortium of bacteria described herein. The compost will be maintained at conditions that include too much water or moisture, include too much nitrogen, lack nitrogen, lack carbon, have an imbalance between nitrogen and carbon, have poor aeration, are unmixed or poorly mixed, are burnt, lack microbes or other life forms, are too compressed, or are otherwise present in poor conditions before, during, and/or after being inoculated with the consortium of bacteria. The consortium of bacteria will be sprayed onto the compost pile, introduced as a powder, or introduced as a liquid. The compost piles will be maintained at individual homes or sites, or at municipal or communal sites. The compost pile will incubate with the consortium of bacteria for about 1 week, about 2 weeks, about 3 weeks, about 4 weeks, about 5 weeks, about 6 weeks, about 7 weeks, about 8 weeks, or longer. While on the compost, the consortium of bacteria will use nitrogen and methane from the ambient air and from the biomass.

The nitrogen fixation rate will be measured in the compost pile by measuring nitrogen fixation of the compost pile over time using acetylene reduction assay and/or gas chromatography. The nitrogen content of the compost will include all nitrogen in the form of ammonia, ammonium, nitrates, nitrites, or any combination thereof. The nitrogen fixation will show an increase in nitrogen content of the compost after incubation with the consortium of bacteria by at least about 1%, at least about 1.5%, at least about 2%, at least about 2.5%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 50%, at least about 100%, at least about 200%, at least about 300%, at least about 400%, at least about 500%, at least about 600%, at least about 700%, at least about 800%, at least about 900%, at least about 1000%, or more as compared to the nitrogen content of a similar compost that was not inoculated with the consortium of bacteria.

The bacterial consortium will consume methane from the ambient air and from the air around and in the compost. The air from the compost will contain methane to provide the carbon source for the consortium of bacteria at a concentration of CH₄ at atmospheric concentrations or at concentrations of at least at least about 2 ppm, at least about 2.5 ppm, at least about 3 ppm, at least about 3.50 ppm, at least about 4 ppm, at least about 4.5 ppm, at least about 5 ppm, at least about 6 ppm, at least about 6.5 ppm, at least about 7 ppm, at least about 7.5 ppm, at least about 8 ppm, at least about 8.50 ppm, at least about 9 ppm, at least about 9.5 ppm, at least about 10 ppm, at least about 15 ppm, at least about 20 ppm, at least about 30 ppm, at least about 40 ppm, at least about 50 ppm, at least about 75 ppm, at least about 100 ppm, at least about 200 ppm, or higher. The input concentration of methane in the air may change over the course of a day, a week, a month, a year, or more.

The compost will be maintained at ambient temperature and pressure and will experience elevated temperatures, decreased temperatures, elevated pressure, and decreased pressure. The input and output concentrations of methane will be measured using gas chromatography or a portable methane detector. The concentrations of methane will be measured at various time points over the course of about 1 day, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, or longer. The concentration of methane will show a reduction in methane of at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% between the input concentration and output concentration of methane as compared to the concentration of methane output from a poorly maintained compost pile that does not comprise the bacterial consortium.

### Example 11: Monetizing Use of the Bacterial Consortium for Credits

The use of the bacterial consortium as disclosed herein will be used to support green initiatives that incentivize companies, governments, organizations, educational institutions, or individuals to increase methane consumption or reduce methane emissions. The bacterial consortium will be applied to any natural or industrial source, such as a farm, a dairy farm, a garden, a compost, indoor or outdoor plants, landfills, oil or gas refineries, waste management facilities, processing plants, chemical processing facilities, exhaust, or wells. The concentration of methane will show a reduction in methane of at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to comparable instances where the bacterial consortium is not used. The concentrations of methane will be measured using gas chromatography or a portable methane detector. The concentrations of methane will be measured at various time points over the course of about 1 day, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, or longer. The concentrations of methane will be measured at various time points over the course of about 1 month, about 2 months, about 3 months, about 4 months, about 5 months, about 6 months, about 7 months, about 8 months, about 9 months, about 10 months, about 11 months, about 12 months, or longer. The reduction in carbon emissions can be exchanged for one or more carbon offset credits, mangrove restoration credits, or other nature offset credits.

### Example 12: Measurement of Nitrogen Fixation by Bacterial Consortium and Species Composition Thereof in Liquid Culture

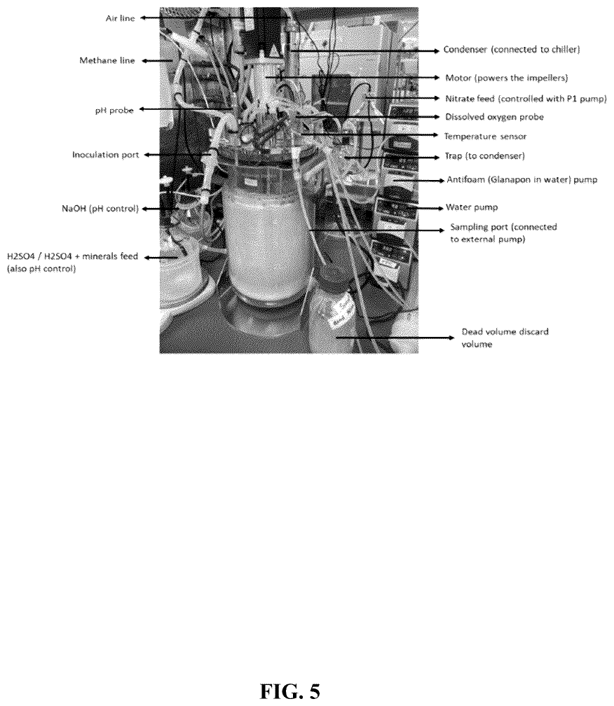

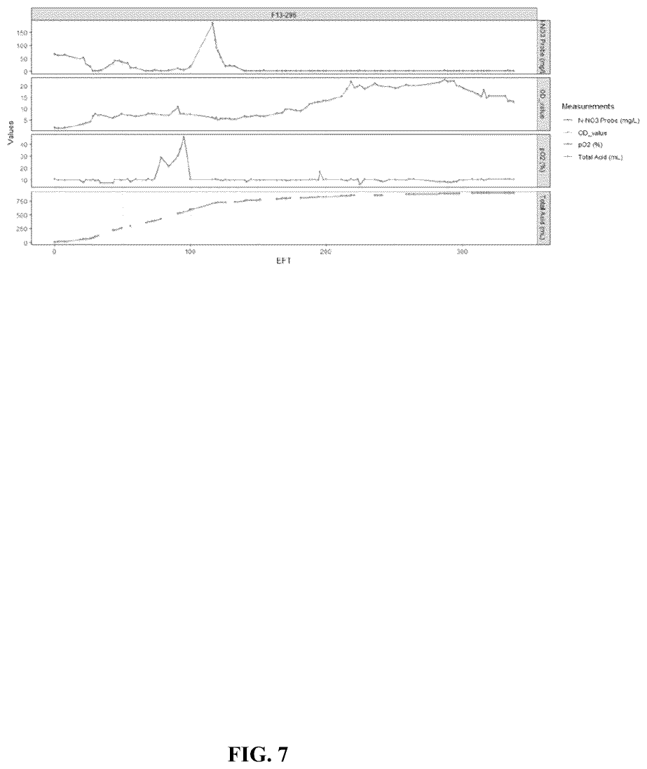

Nitrogen fixation rate and bacterial composition were analyzed over time in different nitrogen environments. The bacterial consortium used was analyzed using the ZymoBIOMICS® Shotgun Metagenomic Sequencing Service for Microbiome as described above. The genus distribution, species distribution and strain distribution results are shown in Tables 12, 13 and 14, respectively. To measure the nitrogen fixation rate of the bacterial consortium, a 4 L bacterial culture was grown in a fermentation tank shown in FIG. 5. A 155 ml bacterial culture was used to seed the growth of approximately 4 L of growth media containing 3962 ml of water, 3 ml of 5M NaN₃, 3 ml of 2M KNO₃, 10.8 ml of Ca feed, 10.8 ml of a macro solution, 4.8 ml of 0.01× TreS/Cu feed, and 6 drops of glanapon. Both temperature (30° C.) and pH were maintained within the fermentation tank throughout the duration of the experiment. Levels of dissolved oxygen (pO₂) and optical density (GD) were measured during the course of the experiment and are shown in FIG. 7. Additionally, the amount of acid added to maintain the pH is shown in FIG. 7.

TABLE 12
Bacterial genus distribution within consortium
over time with different nitrogen environments

| Genus | Inoculum | Nitrate Feeding | Nitrogen Fixation |
|---|---|---|---|
| Sphingopyxis | 0.377886 | 0.573521 | 0.067462 |
| Methylomonas | 0.279896 | 0.064483 | 0.00118 |
| Pseudoxanthomonas | 0.270294 | 0.026799 | 0.00172 |
| Shinella | 0.022024 | 0.118271 | 0.027919 |
| Devosia | 0.011272 | 0.018296 | 0.00115 |
| Planobacterium | 0.009952 | 0.003331 | 0 |
| Ochrobactrum | 0.008332 | 0.025699 | 0.004461 |
| Methylosinus | 0.006121 | 0.015866 | 0.001701 |
| Hyphomicrobium | 0.003961 | 0.007203 | 0.00143 |
| Agrobacterium | 0.00222 | 0.02732 | 0.010713 |
| Reyranella | 0.00165 | 0.003771 | 0.00103 |
| Mesorhizobium | 0.00154 | 0.0013 | 0 |
| Hydrogenophaga | 0.00151 | 0 | 0 |
| Acidovorax | 0.00102 | 0.014055 | 0.008273 |
| Bosea | 0.00096 | 0.009103 | 0 |
| Kaistia | 0.00077 | 0.002501 | 0.00048 |
| Ancylobacter | 0.00059 | 0.00102 | 0 |
| Achromobacter | 0 | 0 | 0.00123 |
| Allorhizobium | 0 | 0 | 0.001681 |
| Enterococcus | 0 | 0.00037 | 0.00039 |
| Flavobacterium | 0 | 0 | 0.013604 |
| Kaistella | 0 | 0.065903 | 0.014525 |
| Methylococcus | 0 | 0.017986 | 0.839239 |
| Stenotrophomonas | 0 | 0 | 0.00012 |
| Variovorax | 0 | 0.003201 | 0.001691 |

TABLE 13
Bacterial species distribution within consortium
over time with different nitrogen environments

| Species | Inoculum | Nitrate Feeding | Nitrogen Fixation |
|---|---|---|---|
| Achromobacter spanius | 0 | 0 | 0.00123 |
| Acidovorax sp | 0.00102 | 0.014055 | 0.008273 |
| Agrobacterium pusense | 0.00222 | 0.02732 | 0.010713 |
| Allorhizobium sp | 0 | 0 | 0.001681 |
| Ancylobacter rudongensis | 0.00059 | 0.00102 | 0 |
| Bosea robiniae | 0.00096 | 0.009103 | 0 |
| Chryseobacterium faecale | 0 | 0.051008 | 0.011314 |
| Chryseobacterium sp | 0.003611 | 0.00141 | 0 |
| Devosia sp | 0.011272 | 0.018296 | 0.00115 |
| Enterococcus gallinarum | 0 | 0.00037 | 0.00039 |
| Flavobacteriales bacterium UBA6726 | 0 | 0.006342 | 0.00145 |
| Flavobacteriia bacterium | 0 | 0.008553 | 0.001761 |
| Flavobacterium lindanitolerans | 0 | 0 | 0.013604 |
| Hydrogenophaga sp | 0.00151 | 0 | 0 |
| Hyphomicrobium zavarzinii | 0.003961 | 0.002861 | 0.00027 |
| Kaistia sp | 0.00077 | 0.002501 | 0.00048 |
| Marnyiella aurantia | 0.006341 | 0.001921 | 0 |
| Marnyiella aurantia | 0 | 0.004342 | 0.00116 |
| Mesorhizobium australicum | 0.00154 | 0.0013 | 0 |
| Methylococcus capsulatus | 0 | 0.017986 | 0.839239 |
| Methylomonas methanica | 0.251 | 0.0579 | 0.00118 |
| Methylomonas sp | 0.028896 | 0.006582 | 0 |
| Methylosinus trichosporium | 0.006121 | 0.015866 | 0.001701 |
| Ochrobactrum teleogrylli | 0.008332 | 0.025699 | 0.004461 |
| Pseudoxanthomonas jiangsuensis | 0.004021 | 0.00118 | 0.00019 |
| Pseudoxanthomonas mexicana | 0.266273 | 0.025619 | 0.00153 |
| Reyranella sp | 0.00165 | 0.003771 | 0.00103 |
| Shinella granuli | 0.022024 | 0.118271 | 0.027919 |
| Sphingopyxis sp | 0.039828 | 0.025109 | 0.00085 |
| Sphingopyxis terrae | 0.338058 | 0.548412 | 0.066611 |
| Stenotrophomonas maltophilia | 0 | 0 | 0.00012 |
| Variovorax sp | 0 | 0.003201 | 0.001691 |

TABLE 14
Bacterial strain and/or species distribution within consortium
over time with different nitrogen environments

| Accession number | Strain/Species | Inoculum | Nitrate Feeding | Nitrogen Fixation |
|---|---|---|---|---|
| GCF_019078745.1 | Sphingopyxis terrae subsp. | 0.186967 | 0.310079 | 0.038332 |
| | terrae YC-JH3 | | | |
| GCF_001644115.1 | Methylomonas methanica | 0.182787 | 0.042195 | 0.00086 |
| | R-45371 | | | |
| GCF_014211895.1 | Pseudoxanthomonas | 0.179256 | 0.017336 | 0.00029 |
| | mexicana GTZY | | | |
| GCF_001644035.1 | Methylomonas methanica | 0.068214 | 0.015705 | 0.00032 |
| | R-45363 | | | |
| GCF_010093385.1 | Pseudoxanthomonas | 0.059872 | 0.005652 | 0.00012 |
| | mexicana DSM 17121 | | | |
| GCF_002277025.1 | Sphingopyxis sp. GW247- | 0.039828 | 0.025109 | 0.00085 |
| | 27LB | | | |
| GCF_000371385.1 | Sphingopyxis sp. MC1 | 0.032146 | 0 | 0 |
| GCF_014397415.1 | Pseudoxanthomonas | 0.027145 | 0.002631 | 0.00112 |
| | mexicana GTZY2 | | | |
| GCF_001610975.1 | Sphingopyxis terrae subsp. | 0.021394 | 0.035522 | 0.006462 |
| | terrae NBRC 15098 203-1 | | | |
| GCF_000365425.1 | Methylomonas sp. MK1 | 0.019264 | 0.004372 | 0 |
| GCF_001652565.1 | Shinella sp. HZN7 | 0.017994 | 0.096524 | 0.022767 |
| GCF_000515215.1 | Methylomonas sp. 11b | 0.009632 | 0.002211 | 0 |
| GCF_001468495.1 | Sphingopyxis sp. A083 | 0.008642 | 0.054339 | 0.004111 |
| GCF_017815095.1 | Marnyiella aurantia cx-311 | 0.006341 | 0.001921 | 0 |
| GCF_002752655.1 | Methylosinus trichosporium | 0.006121 | 0.015866 | 0.001701 |
| | OB3b | | | |
| GCF_013087535.1 | Ochrobactrum soli MTP- | 0.005871 | 0.018286 | 0.003321 |
| | C0764 | | | |
| GCF_004341885.1 | Shinella granuli DSM | 0.004031 | 0.021748 | 0.005152 |
| | 18401 | | | |
| GCF_010093185.1 | Pseudoxanthomonas | 0.004021 | 0.00118 | 0.00019 |
| | jiangsuensis DSM 22398 | | | |
| GCF_000383415.1 | Hyphomicrobium zavarzinii | 0.003721 | 0.002721 | 0.00027 |
| | ATCC 27496 ZV-622 | | | |
| GCF_008831505.1 | Chryseobacterium sp. SNU | 0.003611 | 0.00141 | 0 |
| | WT7 | | | |
| GCF_900243435.1 | Ochrobactrum | 0.00157 | 0.004812 | 0.00079 |
| | haematophilum FI11154 | | | |
| GCF_900177325.1 | Mesorhizobium australicum | 0.00154 | 0.0013 | 0 |
| | B5P | | | |
| GCF_003667905.1 | Agrobacterium tumefaciens | 0.0015 | 0.018506 | 0.007622 |
| | 12D1 | | | |
| GCF_006714945.1 | Bosea sp. AK1 | 0.00096 | 0.007743 | 0 |
| GCF_003634645.1 | Acidovorax sp. 94 | 0.00078 | 0.002521 | 0.0015 |
| GCF_006376685.1 | [Ochrobactrum] teleogrylli | 0.00066 | 0.002021 | 0.00035 |
| | LCB8 | | | |
| GCA_{——}003963445.1 | Ancylobacter aquaticus | 0.00059 | 0.00102 | 0 |
| | UV5 | | | |
| GCF_012272675.1 | Agrobacterium pusense | 0.0005 | 0.002071 | 0.00018 |
| | FDAARGOS_633 | | | |
| GCF_019218805.1 | Acidovorax sp. sif0632 | 0.00024 | 0.007973 | 0.004942 |
| GCF_003664555.1 | Ochrobactrum soli BO-7 | 0.00023 | 0.00058 | 0 |
| GCA_{——}014190925.1 | Rhizobium sp. RAS22 | 0.00022 | 0.003841 | 0.00039 |
| GCF_000008325.1 | Methylococcus capsulatus | 0 | 0.017986 | 0.839239 |
| | Bath | | | |
| GCF_019195395.1 | Chryseobacterium faecale | 0 | 0.051008 | 0.011314 |
| | F4 | | | |
| GCF_001426505.1 | Variovorax sp. Root473 | 0 | 0.003201 | 0.001691 |
| GCF_003053405.1 | Agrobacterium pusense | 0 | 0 | 0.00151 |
| | KCJK7997 | | | |
| GCF_001189595.1 | Achromobacter spanius | 0 | 0 | 0.00112 |
| | CGMCC9173 | | | |
| GCF_001411535.1 | Acidovorax sp. SD340 | 0 | 0.00129 | 0.00069 |
| GCF_014138475.1 | Agrobacterium sp. RC10-4-1 | 0 | 0.001 | 0.00068 |
| GCF_002846575.1 | Flavobacterium | 0 | 0 | 0.00059 |
| | lindanitolerans IP-10 | | | |
| GCF_003633835.1 | Acidovorax sp. 93 | 0 | 0.00032 | 0.00047 |
| GCF_003031575.1 | Agrobacterium pusense S2 | 0 | 0 | 0.00033 |
| GCA_002799235.1 | Stenotrophomonas | 0 | 0 | 0.00012 |
| | maltophilia E729 | | | |
| GCF_003994415.1 | Achromobacter spanius | 0 | 0 | 0.00011 |
| | strain UQ283 | | | |
| GCA_017357485.1 | Rhizobium sp. ZX09 | 0 | 0.00088 | 0 |
| GCF_001429415.1 | Bosea sp. Root670 | 0 | 0.00079 | 0 |
| GCF_013267155.1 | Agrobacterium pusense | 0 | 0.00064 | 0 |
| | FDAARGOS_618 | | | |
| GCF_000304595.1 | Bradyrhizobium lupini | 0 | 0.00038 | 0 |
| | HPC(L) | | | |
| GCF_900102525.1 | Bosea robiniae DSM 26672 | 0 | 0.00034 | 0 |
| GCF_013376875.1 | Acidovorax sp. JMULE5 | 0 | 0.00025 | 0 |
| GCA_017307955.1 | Bosea sp. (in: a- | 0.00077 | 0.002501 | 0.00048 |
| | proteobacteria) | | | |
| GCA_018993155.1 | Hydrogenophaga sp. | 0.00151 | 0 | 0 |
| GCA_002281325.1 | Rhodospirillales bacterium | 0.00165 | 0.003771 | 0.00103 |
| | 35-66-84 | | | |
| GCA_016793385.1 | Hyphomicrobium zavarzinii | 0.00024 | 0.00014 | 0 |
| GCA_014763605.1 | Sphingopyxis terrae | 0.013813 | 0.019137 | 0.002341 |
| GCA_018821845.1 | Alphaproteobacteria | 0.011272 | 0.018296 | 0 |
| | bacterium | | | |
| GCA_018993105.1 | Sphingopyxis terrae | 0.009792 | 0.021337 | 0.002551 |
| GCA_002862825.1 | Sphingomonadaceae | 0.065303 | 0.107998 | 0.012814 |
| | bacterium | | | |
| GCA_001899155.1 | Flavobacterium sp. 38-13 | 0 | 0 | 0.009083 |
| GCA_004799385.1 | Flavobacterium johnsoniae | 0 | 0 | 0.002661 |
| GCA_015999765.1 | Flavobacteriia bacterium | 0 | 0.008553 | 0.001761 |
| GCA_003527225.1 | Rhizobium sp. | 0 | 0 | 0.001681 |
| GCA_002454095.1 | Flavobacteriales bacterium | 0 | 0.006342 | 0.00145 |
| | UBA6726 | | | |
| GCA_017305835.1 | Hyphomicrobiales | 0 | 0.004342 | 0.00116 |
| | bacterium | | | |
| GCA_018828145.1 | Alphaproteobacteria | 0 | 0 | 0.00115 |
| | bacterium | | | |
| GCA_016787825.1 | Flavobacterium | 0 | 0 | 0.00098 |
| | lindanitolerans | | | |
| GCA_018382795.1 | Enterococcus gallinarum | 0 | 0.00037 | 0.00039 |
| GCA_001770675.1 | Burkholderiales bacterium | 0 | 0.00053 | 0.00029 |
| | GWA2_64_37 | | | |
| GCA_003241955.1 | Flavobacteriaceae | 0 | 0 | 0.00029 |
| | bacterium | | | |
| GCA_001772145.1 | Burkholderiales bacterium | 0 | 0.00036 | 0.00021 |
| | RIFCSPLOWO2_12_FULL_64_33 | | | |
| GCA_001897585.1 | Burkholderiales bacterium | 0 | 0 | 0.00017 |
| | 64-34 | | | |
| GCA_001770745.1 | Burkholderiales bacterium | 0 | 0.00081 | 0 |
| | RIFCSPHIGHO2_01_FULL_64_960 | | | |
| GCA_900468845.1 | Rhizobiales bacterium | 0 | 0.00023 | 0 |

The total experiment duration comprised over 350 hours of bacterial growth. During the first 100 hours, NaNO₃ was continuously added to the bacterial consortium through the nitrate feed line shown in FIG. 5. A sample of the bacterial consortium was extracted within the first 100 hours to assess the bacterial composition. The extracted sample was processed and analyzed with the ZymoBIOMICS® Shotgun Metagenomic Sequencing Service for Microbiome as described above. The results are shown in Tables 12-14 for the genus, species and strain distribution, respectively. As shown, the genus, species and strain distribution changed during the fermentation process relative to the inoculation composition while relatively high levels of nitrogen were added to the culture.

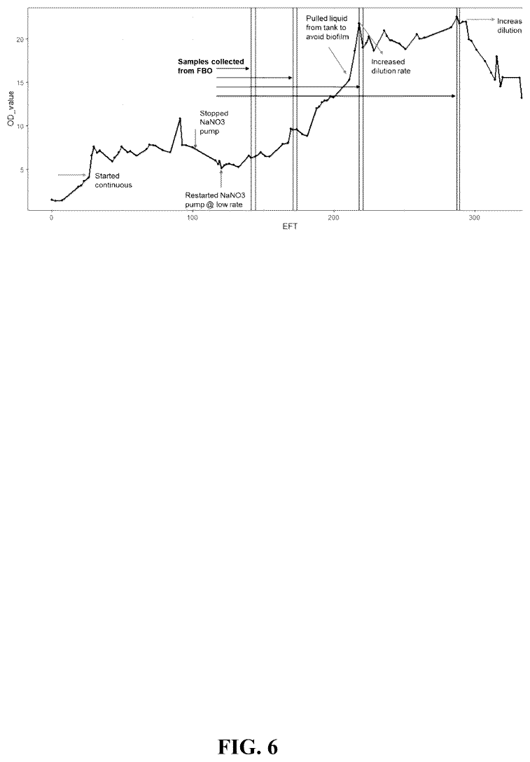

At approximately 100 hours, the NaNO₃ source was turned off for approximately 20 hours. Subsequently, the NaNO₃ was again continuously added, but at a lower concentration. This NaNO₃ concentration was maintained for the remainder of the bacterial growth. A sample of the bacterial consortium was extracted at the end of the experiment to analyze the species composition using the ZymoBIOMICS® Shotgun Metagenomic Sequencing Service for Microbiome as described above. The results of this assessment are shown in Table 12-14, for the genus, species and strain distribution, respectively. As shown, the genus, species and strain distribution changed again relative to both the time points taken. The amount of NaNO₃ added during the course of the experiment is shown in the trace in FIG. 7. Access to lower quantities of nitrate caused the bacterial consortium to use nitrogen fixation as a source of nitrogen. To measure the extent of nitrogen fixation, four samples of the bacteria were collected at the time points indicated on the chart in FIG. 6 and for the duration as noted in Table 15. Each bacteria sample was weighed and lyophilized to remove water and then analyzed for % N content using a Dumas combustion method. The results are listed in Table 15. Nitrogen fixation was calculated based on the % N content from the bacteria samples and the amount of NaNO₃ added to the culture. The cell dried weight (DW) of each sample was divided by the time of sample collection and multiplied by the % N as determined by the Dumas combustion method to generate an N-harvest value (g-N/h). The amount of nitrogen supplied during the course of each time collection step from NaNO₃ was measured by weighing the bottle containing NaNO₃ leading into the fermentation tank and normalizing this quantity to g/hour of nitrogen to generate an N-feed value (g-N/h). The quantity of nitrogen fixation was calculated by subtracting the N-feed value from the N-harvest value at each time point. Nitrogen fixation was also determined based on a % total. Data for each of these calculations is shown in Table 15. As shown, the amount of nitrogen fixation ranged from 0.041-0.267 g/hour and the % nitrogen fixation ranged from 65.9%-89.5% during the course of this experiment.

TABLE 15
Nitrogen fixation data and calculations

| | Time | | % N | | | | |
|---|---|---|---|---|---|---|---|
| | collection | DW | from | N-Harvest | N-Feed | N-Fixation | Nitrogen |
| Sample | (hr) | (g) | DW | (g-N/hr) | (g-N/hr) | (g-N/hr) | fixation % |
| 1 | 3.16 | 3.53 | 5.54 | 0.062 | 0.021 | 0.041 | 65.9 |
| 2 | 2.78 | 3.32 | 6.47 | 0.077 | 0.025 | 0.052 | 67.6 |
| 3 | 2.22 | 4.89 | 8.1 | 0.178 | 0.028 | 0.150 | 84.0 |
| 4 | 1.78 | 5.86 | 9.07 | 0.298 | 0.031 | 0.267 | 89.5 |

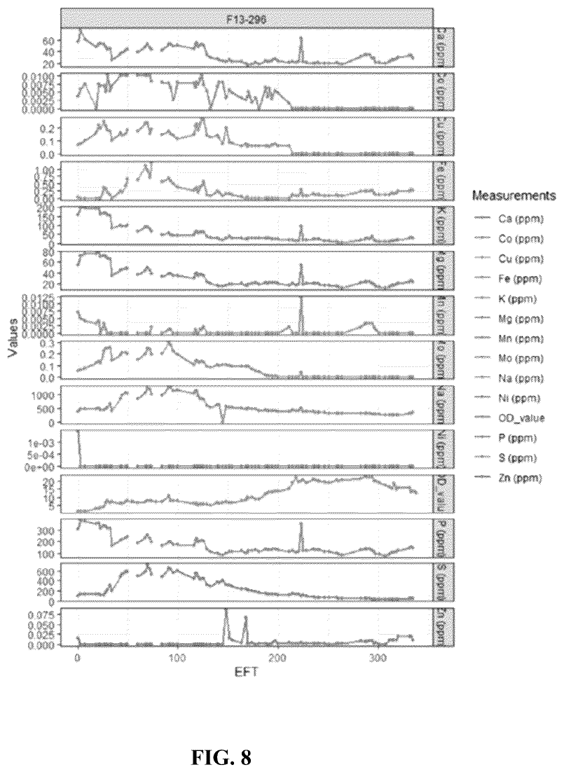

The levels of certain elements present in the media were also measured during the course of this experiment. The concentration of 14 elements at different time points throughout the experiment are shown in FIG. 8. These measurements were obtained by inductively coupled plasma—optical emission spectrometry (ICP-OES). As shown, the levels of certain minerals, including Co, Cu, Fe, Zn, Mn, Ni and Mo, were depleted during the time period of high growth and high nitrogen fixation. This indicates that the bacteria consortium consumes these materials while it is fixing nitrogen.

Culture samples collected from three different time points from the fermentation run analyzed by shotgun metagenomic sequencing (Zymo Research Group) to characterize bacterial compositions. The three different time points include the beginning of the fermentation (“inoculum”), under nitrate feeding condition, and under nitrogen fixation condition. The relative abundances of bacteria within the bacterial consortium changed during the fermentation run. For the inoculum, the top three strains were Sphingopyxis terrae subsp. terrae YC-JH3, Methylomonas methanica R-4537, and Pseudoxanthomonas mexicana GTZY and their relative abundances were 18%, 18%, and 17% respectively. Under nitrate feeding condition, the top three strains/species were Sphingopyxis terrae subsp. terrae YC-JH3, Sphingomonadaceae bacterium, Shinella sp. HZN7 and their relative abundances were 31%, 17%, and 9.6% respectively. Under nitrogen fixation condition, 83% of the bacterial consortium was Methylococcus capsulatus Bath, which was present around 1.7% under nitrate feeding condition.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Example 13: Enrichment of Bacterial Consortium Samples

The composition of bacterial consortium samples were analyzed over time after altering the incubation conditions. Two different bacterial consortium samples were assembled in the following ways. The first sample, termed WBC001, was initiated by combining 2 ml of a frozen culture with 200 ml of a culture medium and placed in a 500 ml glass jar covered with a loose lid. The second sample, termed WBC002, was initiated by combining 10 g of compost, 20 g of Methylosinus trichosporium OB3b culture and 180 g of culture medium into 200 ml. The culture medium in both samples consisted of 0.2 g/L magnesium sulfate heptahydrate (MgSO4·7H₂O), 0.02 g/L calcium chloride dihydrate (CaCl₂·2H₂O), 0.32 g/L monopotassium phosphate (KH₂PO₄), 0.59 g/L dibasic potassium phosphate (K₂HPO₄), 0.1 g/L sodium bicarbonate (NaHCO₃), 1 ml of ethylenediaminetetraacetic acid ferric sodium salt (FeNaEDTA) stock solution (1000×), and 1 ml of trace metal stock solution (1000×). The trace metal stock solution (1000×) consisted of 0.04 g/L sodium molybdate dihydrate (Na₂MoO₄·2H₂O), 0.2 g/L copper(II) sulfate pentahydrate (CuSO₄·5H₂O), 0.4 g/L zinc sulfate heptahydrate (ZnSO₄·7H₂O), 0.02 g/L manganese(II) chloride tetrahydrate (MnCl₂·4H₂O), 0.05 g/L cobalt(II) chloride hexahydrate (CoCl₂·6H₂O), 0.01 g/L nickel(II) chloride hexahydrate (NiCl₂·6H₂O), 0.015 g/L boric acid (H₃BO₃), and 0.25 g/L ethylenediaminetetraacetic acid (EDTA) in deionized water. The FeNaEDTA stock solution (1000×) was made of 0.0065 g/L FeNaEDTA in deionized water. The pH of the medium was around 7. Both culture samples were grown under ambient outdoor conditions in an enclosed environment connected to a natural gas line maintaining approximately 1000-8000 ppm methane in the air. The culture was aerated by continuous bubbling and maintained in the dark.

Both samples were grown under ambient outdoor conditions for 4 weeks. Aliquots of each sample were taken initially, and after both two and four weeks of growth under ambient outdoor conditions. Subsequently, 25 mL of each culture were placed into separate 125 mL serum bottles and fed 25 mL of 99.9% methane every 48 hours for a total of 18 days. The starting OD of the WBC002 sample in the serum bottle was 0.08 and starting OD of the WBC001 sample in the serum bottle was 0.45. After 18 days, WBC002 and WBC001 cultures grew up to 1.8 and 1.4, respectively. Each of the serum bottle grown cultures were used to inoculate a 100 mL culture in a shake flask with a starting OD 0.2. The flasks were fed with 50 mL methane every 48 hours and grown at 30° C. with 250 rpm shaking. Once they reached an OD=1, they were next grown in 1 L shake flask with 125 mL culture (starting OD 0.4) with 25 mL per min continuous methane/air (50%/50%) feeding. After both sample flasks grown up to OD 6-8, aliquots of each sample, termed ‘batch 1’ were taken and stored at −80° C. After 6 months of storage, the frozen batch 1 aliquots of WBC001 and WBC002 were grown in 1 L shake flask with continuous methane/air feeding as described above. Once the OD reached between 6 and 8, aliquots of each consortium sample were stored at −80° C. These aliquots are termed ‘batch 2’. Aliquots of each sample were also taken from serum bottles after 11 days and 18 days of incubation at 30° C. with agitation as described above.

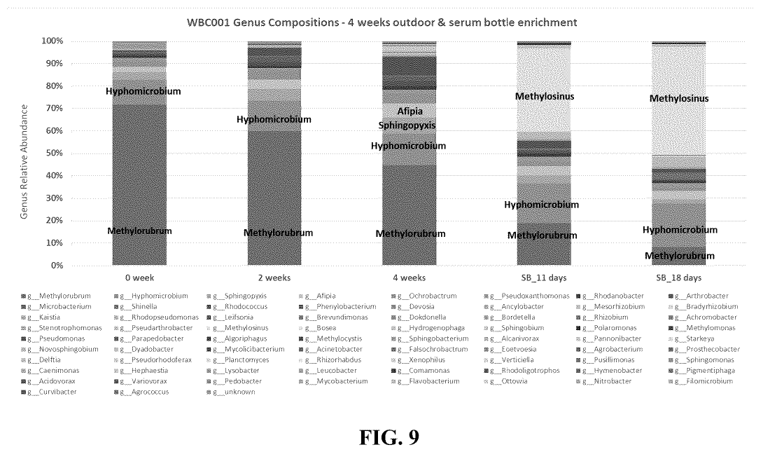

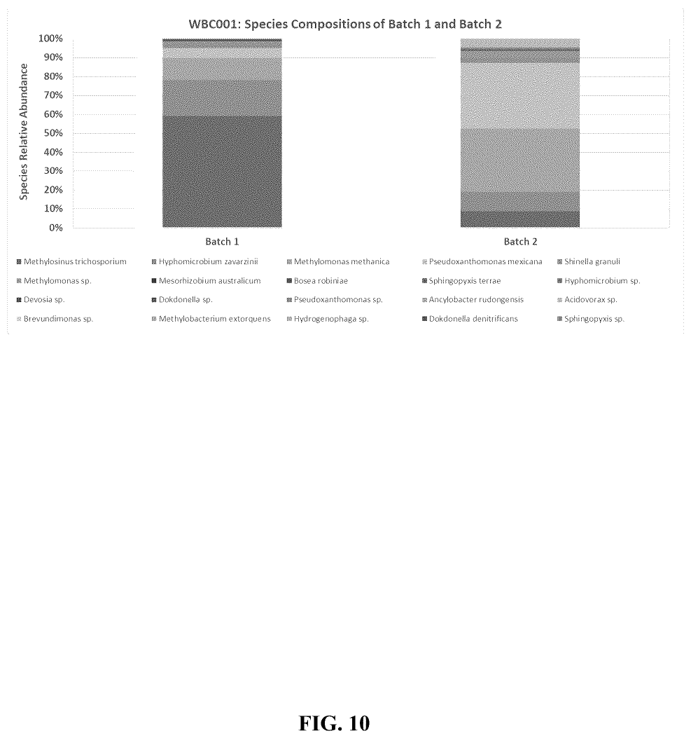

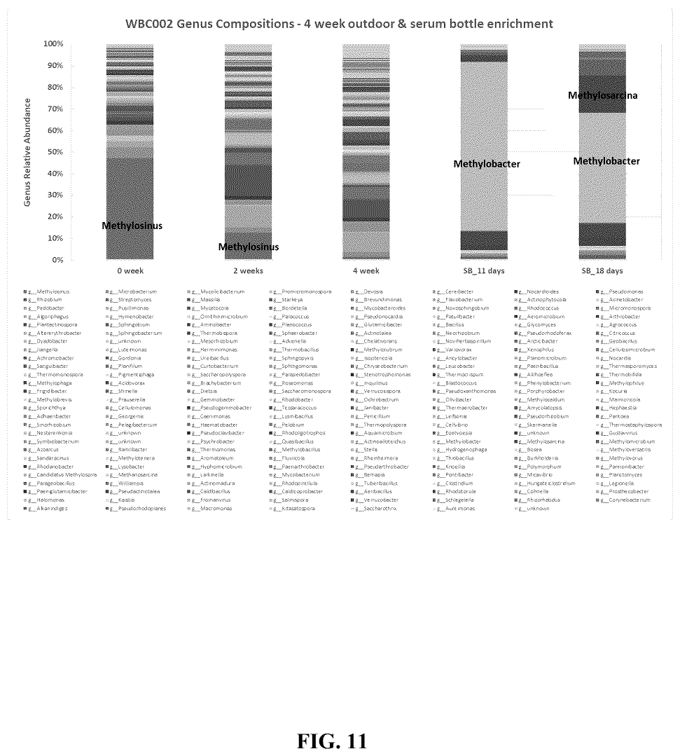

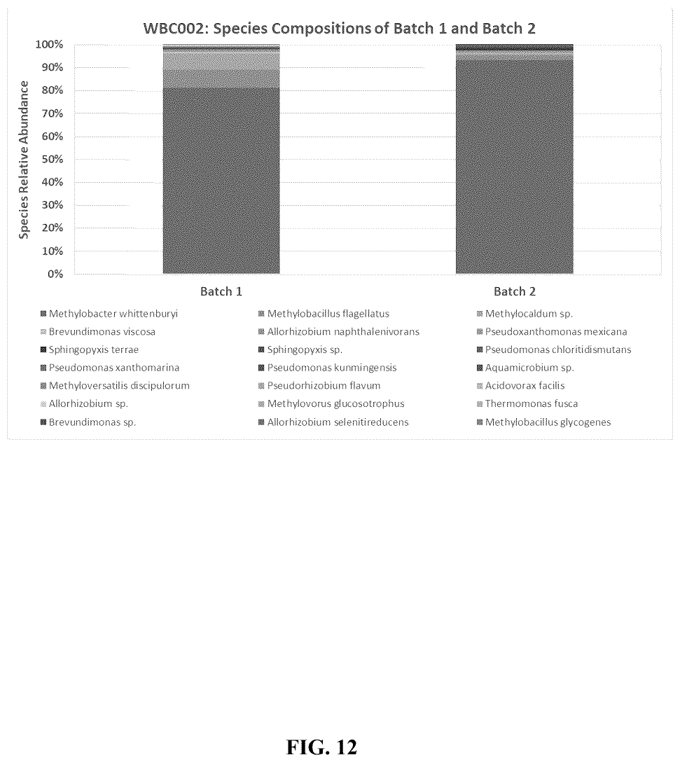

Each of the extracted aliquots was analyzed using metagenomic sequencing as previously described. The genus distribution of the WBC001 bacterial consortium is shown in FIG. 9 for all aliquots taken. Two batches of WBC001 were grown in 1 L shake flasks as descried above. Each of these batches was analyzed using metagenomic sequencing and the relative species abundance is shown in FIG. 10 and Table 16 below. Similarly, the genus distribution of WBC002 bacterial consortium is shown in FIG. 11 for all aliquots taken. Two batches of WBC002 were grown in 1 L shake flasks as described above. Each of these batches was analyzed using metagenomic sequencing and the relative species abundance is shown in FIG. 12 and Table 17 below.

TABLE 16
Bacterial species/strain relative abundance of two different batches of WBC001

| | | WBC001 | WBC001 |
|---|---|---|---|
| Accession number | Strains/Species | batch 1 | batch 2 |
| GCF_002752655.1 | Methylosinus trichosporium OB3b | 0.58853 | 0.08567 |
| GCF_000383415.1 | Hyphomicrobium zavarzinii ATCC 27496 ZV-622 | 0.18023 | 0.09454 |
| GCF_001644115.1 | Methylomonas methanica R-45371 | 0.08460 | 0.24548 |
| GCF_014211895.1 | Pseudoxanthomonas mexicana GTZY | 0.03622 | 0.07671 |
| GCF_001644035.1 | Methylomonas methanica R-45363 | 0.03113 | 0.09239 |
| GCF_001652565.1 | Shinella sp. HZN7 | 0.01779 | 0.02044 |
| GCF_010093385.1 | Pseudoxanthomonas mexicana DSM 17121 | 0.01201 | 0.03428 |
| GCA_016793385.1 | Hyphomicrobium zavarzinii | 0.01076 | 0.00612 |
| GCF_000365425.1 | Methylomonas sp. MK1 | 0.00857 | 0.02419 |
| GCF_014397415.1 | Pseudoxanthomonas mexicana GTZY2 | 0.00570 | 0.23485 |
| GCF_000515215.1 | Methylomonas sp. 11b | 0.00446 | 0.01368 |
| GCF_004341885.1 | Shinella granuli DSM 18401 | 0.00411 | 0.00477 |
| GCF_900177325.1 | Mesorhizobium australicum B5P | 0.00301 | 0 |
| GCF_006714945.1 | Bosea sp. AK1 | 0.00178 | 0 |
| GCF_900117445.1 | Hyphomicrobium sp. NDB2Meth4 | 0.00148 | 0 |
| GCA_018821845.1 | Alphaproteobacteria bacterium | 0.00120 | 0 |
| GCF_019078745.1 | Sphingopyxis terrae subsp. terrae YC-JH3 | 0.00119 | 0.00595 |
| GCA_002483055.1 | Rhodanobacteraceae bacterium UBA7676 | 0.00116 | 0 |
| GCF_001427585.1 | Pseudoxanthomonas sp. Root630 | 0.00108 | 0.00882 |
| GCF_003963445.1 | Ancylobacter aquaticus UV5 | 0.00070 | 0 |
| GCA_019218805.1 | Acidovorax sp. Sif0632 | 0.00054 | 0.02771 |
| GCA_002386585.1 | Brevundimonas sp. UBA4609 | 0.00050 | 0.00198 |
| GCA_{——}018993155.1 | Hydrogenophaga sp. | 0.00050 | 0 |
| GCA_{——}003241385.1 | Rhodanobacter denitrificans | 0.00049 | 0 |
| GCA_002862825.1 | Sphingomonadaceae bacterium | 0.00038 | 0.00194 |
| GCF_000018845.1 | Methylorubrum extorquens PA1 | 0.00035 | 0 |
| GCA_{——}017305595.1 | Hyphomicrobiales bacterium | 0.00020 | 0 |
| GCF_001610975.1 | Sphingopyxis terrae subsp. terrae NBRC 15098 203-1 | 0.00018 | 0.00122 |
| GCF_001429415.1 | Bosea sp. Root670 | 0.00017 | 0 |
| GCA_003446915.1 | Brevundimonas sp. | 0.00017 | 0 |
| GCF_003634645.1 | Acidovorax sp. 94 | 0.00012 | 0.00827 |
| GCF_002277025.1 | Sphingopyxis sp. GW 247-27LB | 0.00012 | 0 |
| GCF_000371385.1 | Sphingopyxis sp. MC1 | 0.00012 | 0 |
| GCA_001423025.1 | Methylobacterium sp. Leaf122 | 0.00010 | 0 |
| GCF_013376875.1 | Acidovorax sp. JMULE5 | 0.00006 | 0 |
| GCF_900100155.1 | Ancylobacter rudongensis CGMCC 1.1761 | 0.00006 | 0 |
| GCF_000083545.1 | Methylorubrum extorquens DM4 | 0.00006 | 0 |
| GCA_014763605.1 | Sphingopyxis terrae | 0.00006 | 0 |
| GCF_001411535.1 | Acidovorax sp. SD340 | 0 | 0.00415 |
| GCF_003633835.1 | Acidovorax sp. 93 | 0 | 0.00115 |

TABLE 17
Bacterial species/strain relative abundance of two different batches of WBC002

| | | WBC002 | WBC002 |
|---|---|---|---|
| Accession Number | Strains/Species | batch 1 | batch 2 |
| GCF_000383855.1 | Methylobacter marinus A45 | 0.649323 | 0.731872 |
| GCF_000745375.1 | Methylobacter whittenburyi UCM-B-3033 | 0.110754 | 0.135649 |
| GCF_017832375.1 | Methylocaldum sp. RMAD-M | 0.060903 | 0.006661 |
| GCF_000746145.1 | Methylobacter sp. BBA5.1 | 0.050531 | 0.063729 |
| GCF_000013705.1 | Methylobacillus flagellatus KT | 0.017894 | 0 |
| GCF_900116065.1 | Brevundimonas viscosa CGMCC 1.10683 | 0.008432 | 0.00129 |
| GCF_002005105.1 | Methylocaldum sp. 14B | 0.006611 | 0.00037 |
| GCF_900492205.1 | Rhizobium naphthalenivorans, T2.26MG-112.21 | 0.004071 | 0.0018 |
| GCF_000385375.1 | Methyloversatilis discipulorum RZ18-153 | 0.003321 | 0 |
| GCF_014397415.1 | Pseudoxanthomonas mexicana GTZY2 | 0.003321 | 0.00093 |
| GCA_{——}009749305.1 | Methylocaldum sp. BRCS4 | 0.003131 | 0.00097 |
| GCF_019078745.1 | Sphingopyxis terrae subsp. terrae YC-JH3 | 0.00223 | 0.005381 |
| GCF_000238595.1 | Acidovorax sp. NO-1 | 0.00134 | 0 |
| GCF_902502825.2 | Rhizobium flavum YW14 | 0.00119 | 0 |
| GCF_000023745.1 | Methylovorus glucosetrophus SIP 3-4 | 0.00115 | 0 |
| GCF_014211895.1 | Pseudoxanthomonas mexicana GTZY | 0.00099 | 0.006671 |
| GCF_005862345.1 | Thermomonas fusca SHC 3-19 | 0.0008 | 0 |
| GCF_010093385.1 | Pseudoxanthomonas mexicana DSM 17121 | 0.00049 | 0.00211 |
| GCF_000518785.1 | Rhizobium selenitireducens ATCC BAA-1503 | 0.00042 | 0 |
| GCF_902153235.2 | Pseudorhizobium halotolerans AB21 | 0.0004 | 0 |
| GCF_000371385.1 | Sphingopyxis sp. MC1 | 0.00039 | 0 |
| GCF_000617925.1 | Methylobacillus glycogenes JCM 2850 | 0.00029 | 0 |
| GCF_001610975.1 | Sphingopyxis terrae subsp. terrae NBRC 15098 203-1 | 0.00025 | 0.00083 |
| GCF_902502895.2 | Rhizobium sp. Khangiran2 | 0.00022 | 0 |
| GCF_002843895.1 | Stutzerimonas stutzeri DSM 50227 | 0 | 0.006481 |
| GCF_013410105.1 | Sphingopyxis sp. JAI108 | 0 | 0.00044 |
| GCF_900114065.1 | Pseudomonas kunmingensis strain DSM 25974 | 0 | 0.00041 |
| GCA_009360475.1 | Methylobacillus sp. | 0.060323 | 0.021933 |
| GCA_014860725.1 | Aquamicrobium sp. | 0.004491 | 0 |
| GCA_003527225.1 | Rhizobium sp. | 0.0011 | 0 |
| GCF_900492185.1 | Ciceribacter selenitireducens ATCC BAA-1503, | 0.00082 | 0 |
| | T2.30D-1.1 | | |
| GCA_002862825.1 | Sphingomonadaceae bacterium | 0.00076 | 0.00107 |
| GCA_001794825.1 | Caulobacterales bacterium | 0.00046 | 0 |
| | RIFCSPHIGHO2_01_FULL_70_19 | | |
| GCA_{——}003248915.1 | Thauera sp. (unverified source organism) | 0.00037 | 0 |
| GCA_902168315.1 | uncultured Novosphingobium sp. | 0.00033 | 0.006271 |
| GCF_903797705.1 | Acidovorax facilis | 0.00027 | 0 |
| GCA_015234495.1 | Brevundimonas sp. | 0.00019 | 0 |
| GCA_018780585.1 | Methyloversatilis discipulorum | 0.00014 | 0 |

As shown in FIGS. 9 and 11, the bacterial consortium in both samples changed both during the outdoor ambient 4 week incubation and then again during the 18 days at 30° C. with agitation. The WBC001 sample started with over 70% composition of methylorubrum, which decreased to under 50% after 4 weeks in outdoor conditions and then to under 10% after an additional 18 days at 30° C. In contrast, the genus having the highest composition at the end of the 18 days at 30° C. was methylosinus, accounting for over 40% of the consortium at the last time point and less than 1% at the beginning. There were similar trends observed with the WBC002 sample, but with different specific genera. Overall, this example demonstrates that certain genera can be enriched over time and the composition can depend on the environmental conditions.

### Example 14: Measurement of Methane Consumption Rate of Bacterial Consortium

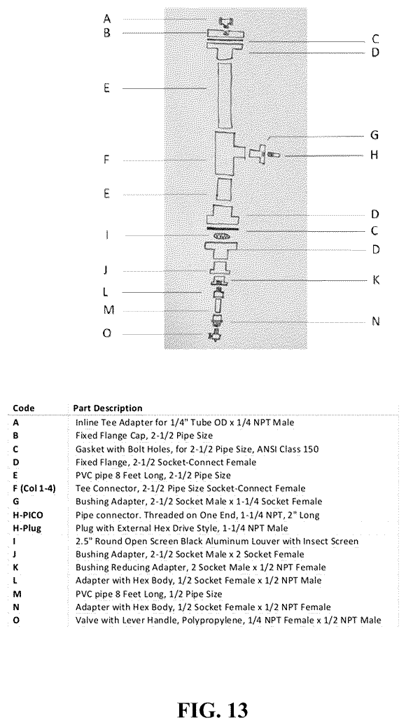

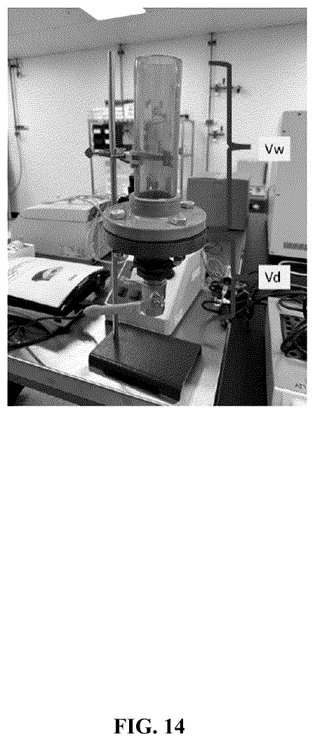

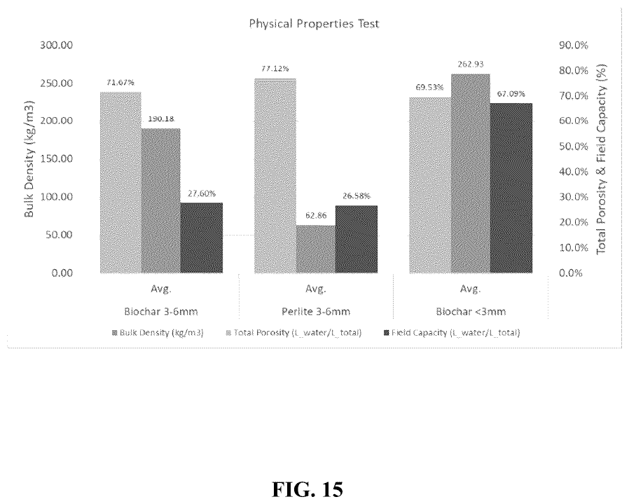

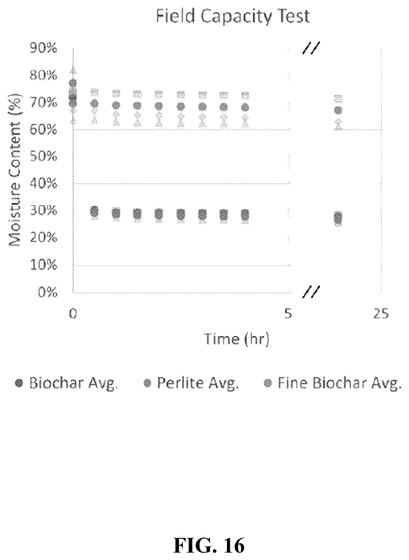

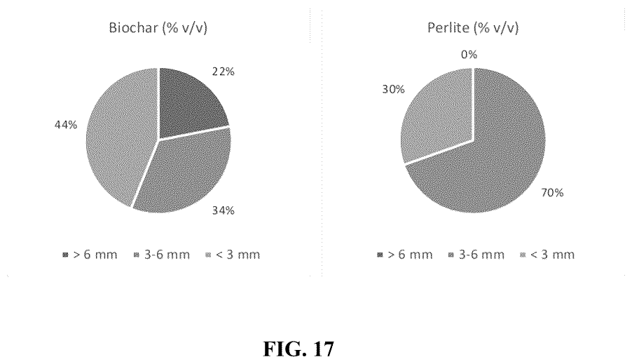

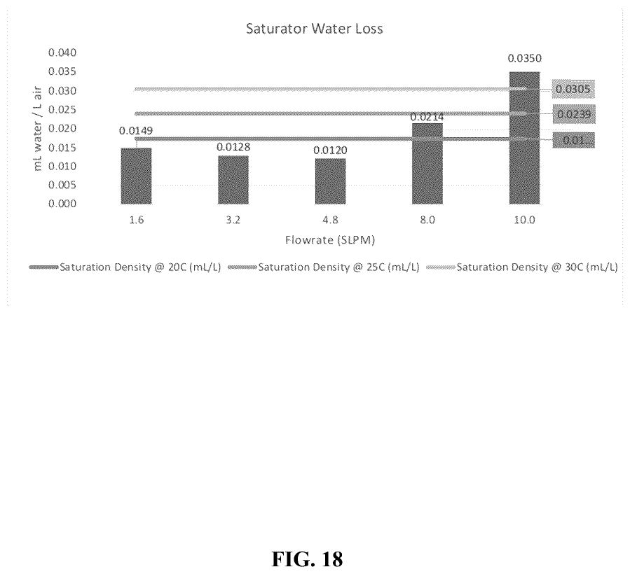

Bacterial consortium samples were loaded into columns containing different matrices within a solid-state bioreactor to measure the methane consumption rate of the bacterial consortium. A solid state bioreactor was constructed as depicted in FIG. 13. Aspects of the bioreactor were analyzed to confirm the physical properties of components and delivery of gases and liquids within it prior to analyzing the methane consumption of the bacterial consortium samples. A physical property testing apparatus was constructed to evaluate these properties, as shown in FIG. 14. Three different columns were constructed with different matrices: Biochar, Perlite and Fine Biochar. Bulk density, total porosity and field capacity (%) were measured for each column and the results are shown in FIG. 15. Moisture content was analyzed at multiple time points over the course of 24 hours for each of the three column materials and the results are shown in FIG. 16. As shown, the fine biochar had the highest moisture content, while all three materials had stable moisture content over the course of the 24-hour period. The pore size was measured for both biochar and perlite and the distribution of sizes is shown in the pie charts of FIG. 17. Finally, optimization of the moisture saturator settings of the bioreactor was performed by varying the flow rate of driven air through the water-holding vessel prior to the feed gas part. The results of these tests are shown in FIG. 18, where the amount water per volume air is plotted against different flow rates. As shown, with a lower flow rate, the feed air reaches at least 70% of the moisture saturation at 20° C.

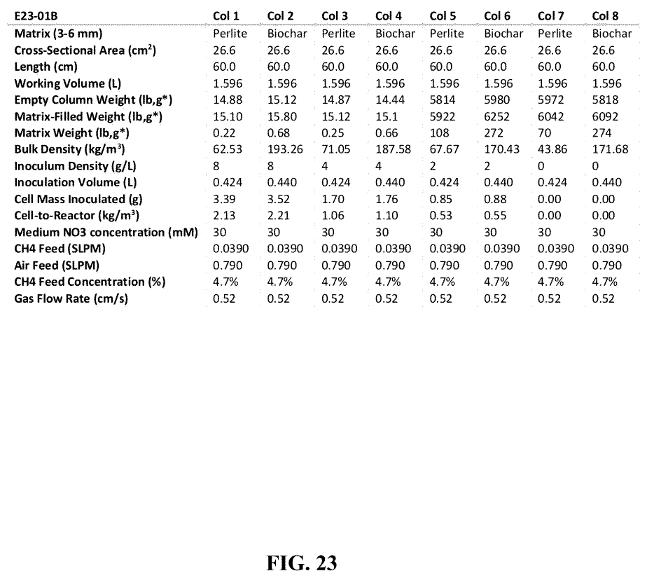

A first experiment was performed according to the details shown in FIG. 23. As shown, each column contained a matrix material, a bacterial consortium and culture broth. The methane consumption rate is calculated according to Equations 1 and 2 below by taking the volumetric portion of the fed methane flow removed, converting this into the mass of methane consumed and normalizing this unit by the working volume of the bioreactor.

$$\begin{matrix}Equation 1 : methane removal efficiency determination & \\ {Methane Removal Efficiencty ( MRE ) = ( {[ C {H}_{4} ]}_{f e e d} - [ CH4 ]}_{o u t} ) {/ [ {CH}_{4} ]}_{f e e d} & \end{matrix}$$

$$\begin{matrix}Equation 2 : methane consumption rate calculation & \\ Methane Consumption Rate = \frac{( \frac{Q CH 4 in XMRE}{V gas Molar Volumne} ) \times Molar Mass CH 4}{V r e a c t o r} & \end{matrix}$$

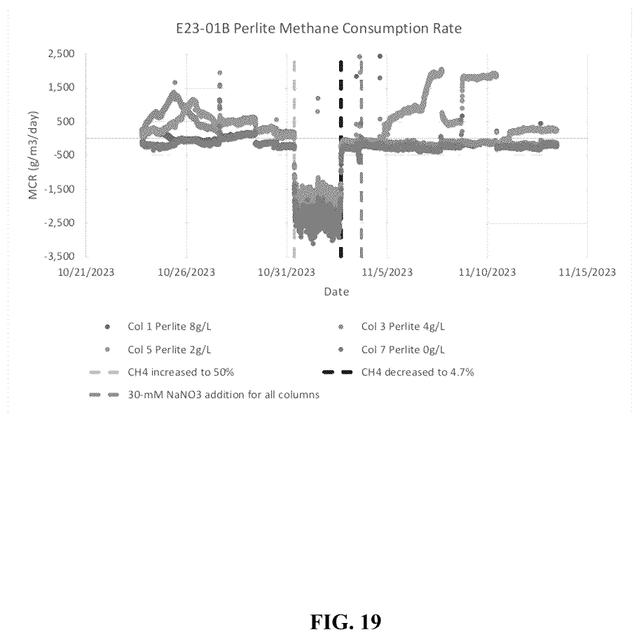

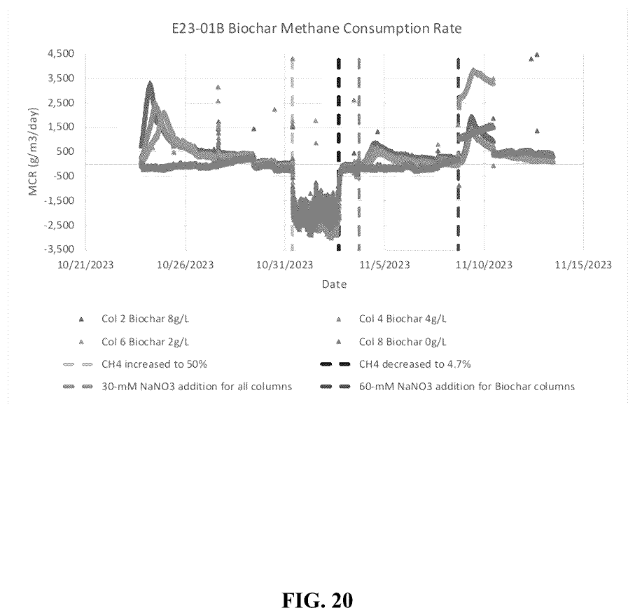

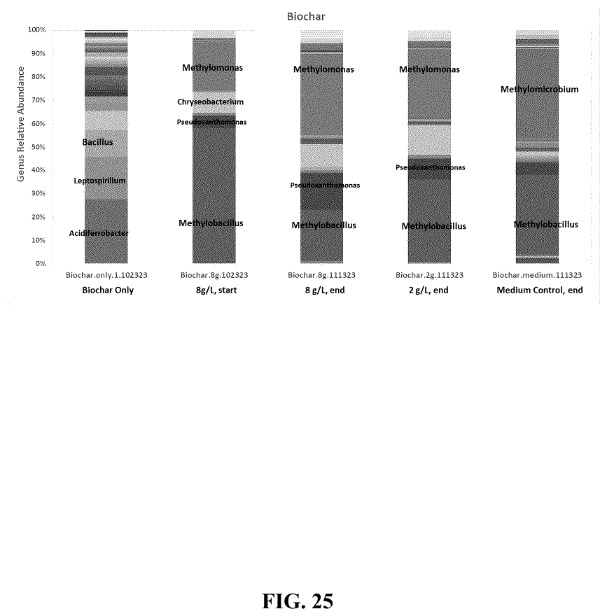

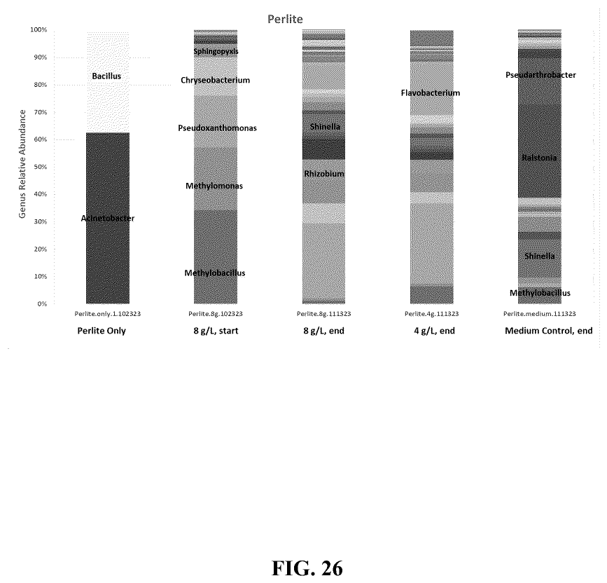

The methane consumption rate was measured over time for each of the bacterial consortiums in each of the eight columns. The results of the perlite containing columns are shown in FIG. 19. This experiment involved altering the methane concentration and adding additional NaNO₃ to each of the columns. As shown, increasing the methane concentration to 50% caused the methane consumption rate to decrease for all samples. For two of the perlite containing columns, the methane consumption rate increased initially and then decreased. This trend was repeated after adding an additional NaNO₃. The results of the biochar containing columns are shown in FIG. 20. Similar to the perlite trends, increasing the methane concentration caused the methane consumption rate to decrease for all samples. In this case, all of the biochar containing samples showed an increase in methane consumption followed by a decrease and this trend was again repeated after the addition of more NaN₃. Full-length 16S sequencing analysis was performed with samples from the beginning and end of the experiment for both the Perlite and Biochar samples. The genus distribution of each of the bacterial consortium samples is shown in FIG. 25 and Table 18 below for the Biochar containing sample and FIG. 26 and Table 19 below for the Perlite containing samples. For each of the matrix types, the genus distribution was different at the beginning compared to the end. Also, the distribution at the end of each experiment across matrix types was different. For both matrix materials, the bacterial consortium sample contained over 50% of the methylomonas and methylobacillus genera by relative abundance at the beginning of each experiment. At the end of the experiment, methylomonas and methylobacillus still comprised over 50% for the Biochar columns but was detected at only very low levels for the Perlite columns. In this experiment, the Biochar matrix was an effective material in supporting the bacterial consortium because the associated consortium had a higher methane consumption rate and retained a high abundance of key bacteria genera responsible for methane consumption

TABLE 18
Bacterial genus distribution of consortium samples
grown with Biochar matrix in bioreactor

| | Biochar.only | Biochar | Biochar | Biochar | Biochar |
|---|---|---|---|---|---|
| Genus | 102323 | 8 g/L. Start | 8 g/L. End | 2 g/L. End | 4 g/L. End |
| Methylobacillus | 0.003079292 | 0.567427007 | 0.216296541 | 0.352299978 | 0.338838174 |
| Methylomonas | 0 | 0.212408759 | 0.349376703 | 0.292783955 | 0 |
| Chryseobacterium | 0 | 0.089279197 | 0.100800793 | 0.128706126 | 0.02 |
| Pseudoxanthomonas | 0.001539646 | 0.052645985 | 0.158589945 | 0.088647264 | 0.053195021 |
| Sphingopyxis | 0 | 0.030018248 | 0.018822752 | 0.016350556 | 0.01966805 |
| Ramlibacter | 0.016936105 | 0.010720803 | 0.003549905 | 0.002370831 | 0.000912863 |
| Rhizobium | 0 | 0.009260949 | 0.026830678 | 0.021637236 | 0.002489627 |
| Achromobacter | 0 | 0.004607664 | 0.004540576 | 0.003460868 | 0.004481328 |
| Brevundimonas | 0 | 0.003421533 | 0.000990671 | 0.000408764 | 0.001825726 |
| Terrimonas | 0 | 0.00314781 | 0.032774705 | 0.029158491 | 0 |
| Acidovorax | 0 | 0.002509124 | 0.008503261 | 0.009783083 | 0.012697095 |
| Sphingomonas | 0.005388761 | 0.002235401 | 0.002724346 | 0.001635056 | 0.00780083 |
| Flavobacterium | 0 | 0.002007299 | 0.008916041 | 0.004550905 | 0.020746888 |
| Flavisolibacter | 0 | 0.001459854 | 0.01502518 | 0.006867233 | 0.003817427 |
| Hydrogenophaga | 0 | 0.001414234 | 0.004705688 | 0.006267713 | 0.004647303 |
| Variovorax | 0 | 0.001368613 | 0.002806902 | 0.001117288 | 0.001327801 |
| Pedobacter | 0 | 0.001140511 | 0.000908115 | 0.000490517 | 0 |
| Parapedobacter | 0 | 0.000912409 | 0.007017254 | 0.002888598 | 0 |
| Ochrobactrum | 0 | 0.000821168 | 0.000577892 | 0.001008284 | 0 |
| Shinella | 0 | 0.000547445 | 0.004210353 | 0.002534336 | 0.018589212 |
| Devosia | 0 | 0.000319343 | 0.00181623 | 0.001962067 | 0.001078838 |
| Parapedobacter- | 0 | 0.000273723 | 0 | 0.000681273 | 0 |
| Sphingobacterium | | | | | |
| Chitinophaga | 0 | 0.000182482 | 0.009824156 | 0.003733377 | 0.006970954 |
| Phenylobacterium | 0 | 0.000182482 | 0 | 0 | 0 |
| Dyadobacter | 0 | 0.000136861 | 0.006109139 | 0.004087639 | 0.009045643 |
| Herbaspirillum | 0.009237875 | 0.00009124 | 0 | 0 | 0 |
| Agrobacterium | 0 | 0.00009124 | 0 | 0 | 0 |
| Acidiferrobacter | 0.274056967 | 0 | 0 | 0 | 0 |
| Leptospirillum | 0.181678214 | 0 | 0 | 0 | 0 |
| Bacillus | 0.114703618 | 0 | 0 | 0 | 0 |
| Pseudolabrys | 0.033102386 | 0 | 0 | 0 | 0 |
| Other | 0.023864511 | 0 | 0.001403451 | 0.001417048 | 0.024481328 |
| Ralstonia | 0.020785219 | 0 | 0 | 0 | 0.00033195 |
| Nitriliruptor | 0.018475751 | 0 | 0 | 0 | 0 |
| Legionella | 0.009237875 | 0 | 0 | 0 | 0 |
| Variibacter | 0.009237875 | 0 | 0 | 0 | 0 |
| Hyphomicrobium | 0.008468052 | 0 | 0.004375464 | 0.001417048 | 0.006721992 |
| Caldalkalibacillus | 0.007698229 | 0 | 0 | 0 | 0 |
| Caldinitratiruptor | 0.007698229 | 0 | 0 | 0 | 0 |
| Bauldia | 0.006158584 | 0 | 0 | 0 | 0 |
| Catelliglobosispora | 0.004618938 | 0 | 0 | 0 | 0 |
| Phyllobacterium | 0.004618938 | 0 | 0 | 0 | 0 |
| Herminiimonas | 0.003849115 | 0 | 0 | 0 | 0 |
| Listeria | 0.003849115 | 0 | 0 | 0 | 0 |
| Patulibacter | 0.003849115 | 0 | 0 | 0 | 0 |
| Aneurinibacillus | 0.003079292 | 0 | 0 | 0 | 0 |
| Geobacillus | 0.003079292 | 0 | 0 | 0 | 0 |
| Acidothermus | 0.002309469 | 0 | 0 | 0 | 0 |
| Gaiella | 0.002309469 | 0 | 0 | 0 | 0 |
| Propionibacterium | 0.001539646 | 0 | 0 | 0 | 0 |
| Amoebophilus | 0 | 0 | 0 | 0.000136255 | 0 |
| Afipia | 0 | 0 | 0 | 0 | 0 |
| Allocatelliglobosispora | 0 | 0 | 0 | 0 | 0 |
| Aquamicrobium | 0 | 0 | 0 | 0 | 0.000165975 |
| Aquicella | 0 | 0 | 0 | 0 | 0 |
| Beggiatoa | 0 | 0 | 0 | 0 | 0.002655602 |
| Bordetella | 0 | 0 | 0.000247668 | 0 | 0 |
| Bradyrhizobium | 0 | 0 | 0 | 0 | 0 |
| Caenimonas | 0 | 0 | 0 | 0.000109004 | 0 |
| Cohnella | 0 | 0 | 0 | 0 | 0 |
| Curvibacter | 0 | 0 | 0 | 0 | 0 |
| Dactylosporangium | 0 | 0 | 0 | 0 | 0 |
| Ferrovibrio | 0 | 0 | 0 | 0 | 0.000248963 |
| Flavihumibacter | 0 | 0 | 0 | 0 | 0.001659751 |
| Gemmata | 0 | 0 | 0 | 0 | 0 |
| Haliangium | 0 | 0 | 0 | 0 | 0 |
| Herbiconiux | 0 | 0 | 0 | 0 | 0 |
| Jishengella | 0 | 0 | 0 | 0 | 0 |
| Kaistia | 0 | 0 | 0 | 0 | 0 |
| Lysobacter | 0 | 0 | 0 | 0.000245258 | 0.01153527 |
| Mesorhizobium | 0 | 0 | 0.001073227 | 0.000545019 | 0.001742739 |
| Methylobacterium | 0 | 0 | 0 | 0 | 0.000414938 |
| Methylomicrobium | 0 | 0 | 0 | 0.009183562 | 0.383983402 |
| Methylopila | 0 | 0 | 0 | 0 | 0.000414938 |
| Myxococcus | 0 | 0 | 0 | 0 | 0 |
| Nocardioides | 0 | 0 | 0 | 0 | 0.000165975 |
| Paenibacillus | 0 | 0 | 0 | 0 | 0.00473029 |
| Planctomyces | 0 | 0 | 0 | 0 | 0.000746888 |
| Pseudarthrobacter | 0 | 0 | 0 | 0 | 0.001908714 |
| Pseudomonas | 0 | 0 | 0 | 0 | 0.000248963 |
| Pseudonocardia | 0 | 0 | 0 | 0 | 0.000414938 |
| Reticulomyxa filosa | 0 | 0 | 0 | 0.000109004 | 0 |
| Reyranella | 0 | 0 | 0.000908115 | 0.000517768 | 0.0006639 |
| Rickettsia | 0 | 0 | 0 | 0 | 0.005975104 |
| Roseimicrobium | 0 | 0 | 0 | 0.000654022 | 0.020829876 |
| Sediminibacterium | 0 | 0 | 0 | 0 | 0 |
| Sphingobacterium | 0 | 0 | 0 | 0 | 0.00033195 |

TABLE 19
Bacterial genus distribution of consortium samples grown with Perlite matrix in bioreactor

| | Perlite.only | Perlite | Perlite | Perlite | Perlite |
|---|---|---|---|---|---|
| Genus | 102323 | 8 g/L. Start | 8 g/L. End | 4 g/L. End | 2 g/L. End |
| Pseudoxanthomonas | 0 | 0.188049309 | 0.272838782 | 0.292717484 | 0.01343525 |
| Flavobacterium | 0 | 0.00231138 | 0.085168791 | 0.185928835 | 0.010956903 |
| Sphingopyxis | 0 | 0.037353042 | 0.084315503 | 0.069829575 | 0.014713555 |
| Rhizobium | 0 | 0.011100331 | 0.077222548 | 0.050358256 | 0.004747991 |
| Terrimonas | 0 | 0.00950234 | 0.073969388 | 0.027527021 | 0.000417406 |
| Chryseobacterium | 0 | 0.140366397 | 0.073702736 | 0.039954661 | 0.001382657 |
| Shinella | 0 | 0.003681087 | 0.066289798 | 0.029470105 | 0.13923093 |
| Achromobacter | 0 | 0.003167447 | 0.028745134 | 0.021211999 | 0.054367108 |
| Brevundimonas | 0 | 0.002710878 | 0.01855901 | 0.011820427 | 0.000443494 |
| Hyphomicrobium | 0 | 0.000884602 | 0.017919044 | 0.016597174 | 0.000469582 |
| Parapedobacter | 0 | 0.002596736 | 0.016639113 | 0.030036838 | 0.000156527 |
| Cohnella | 0 | 0 | 0.014345902 | 0.0496296 | 0 |
| Flavisolibacter | 0 | 0.004508618 | 0.01413258 | 0.011779946 | 0 |
| Ochrobactrum | 0 | 0.002596736 | 0.01306597 | 0.003805206 | 0.006808932 |
| Devosia | 0 | 0.001940418 | 0.012639326 | 0.008258106 | 0.001565272 |
| Other | 0 | 0 | 0.01194603 | 0.00178116 | 0.001121778 |
| Methylobacillus | 0 | 0.340543317 | 0.011839369 | 0.063190706 | 0.060993426 |
| Mesorhizobium | 0 | 0.000428033 | 0.011146072 | 0.008379549 | 0.004461025 |
| Planctomyces | 0 | 0 | 0.010719428 | 0 | 0 |
| Ramlibacter | 0 | 0.005764182 | 0.010612767 | 0.011375137 | 0.000182615 |
| Sphingomonas | 0 | 0.003281589 | 0.009439497 | 0.008339068 | 0.001487008 |
| Methylomonas | 0 | 0.230709965 | 0.007839582 | 0.010039266 | 0 |
| Parapedobacter- | 0 | 0.000627782 | 0.004639753 | 0.003400397 | 0.001408745 |
| Sphingobacterium | | | | | |
| Paenibacillus | 0 | 0 | 0.004479761 | 0.005505404 | 0.001487008 |
| Acidovorax | 0 | 0.003281589 | 0.0043731 | 0.007448488 | 0.026113952 |
| Roseimicrobium | 0 | 0 | 0.0043731 | 0 | 0.000156527 |
| Dyadobacter | 0 | 0 | 0.004266439 | 0.000242885 | 0.0008609 |
| Afipia | 0 | 0 | 0.004053117 | 0.000485771 | 0.002165293 |
| Hydrogenophaga | 0 | 0.001227029 | 0.003573143 | 0.008905801 | 0.009261192 |
| Reyranella | 0 | 0.000142678 | 0.003146499 | 0.002307412 | 0.002008766 |
| Aquamicrobium | 0 | 0 | 0.002666524 | 0.000769137 | 0.001017427 |
| Microbacterium | 0 | 0 | 0.002613194 | 0.001497794 | 0.002947929 |
| Olivibacter | 0 | 0 | 0.002239881 | 0.00311703 | 0 |
| Chitinophaga | 0 | 0.000370962 | 0.001919898 | 0.001052504 | 0.003808828 |
| Sphingobacterium | 0 | 0 | 0.001653245 | 0 | 0.002452259 |
| Devosia-Pelagibacterium | 0 | 0 | 0.001439923 | 0.003764725 | 0 |
| Herbiconiux | 0 | 0 | 0.001173271 | 0.002266931 | 0 |
| Caenimonas | 0 | 0 | 0.000693296 | 0.000566733 | 0.00005217 |
| Variovorax | 0 | 0.000370962 | 0.000639966 | 0.000647695 | 0.008217677 |
| Kaistia | 0 | 0.000114142 | 0.000639966 | 0.000647695 | 0 |
| Phenylobacterium | 0 | 0.00008561 | 0.000639966 | 0.000323847 | 0.018783262 |
| Chthoniobacter | 0 | 0 | 0.000639966 | 0 | 0 |
| Agrobacterium | 0 | 0.00005707 | 0.000586635 | 0.000283366 | 0 |
| Pseudarthrobacter | 0 | 0 | 0.000426644 | 0.000242885 | 0.168084107 |
| Arthrobacter | 0 | 0 | 0.000426644 | 0 | 0.004121883 |
| Eoetvoesia | 0 | 0 | 0.000266652 | 0 | 0.000495669 |
| Pusillimonas | 0 | 0 | 0.000266652 | 0 | 0 |
| Cupriavidus | 0 | 0 | 0.000213322 | 0 | 0.002608786 |
| Bordetella | 0 | 0 | 0.000213322 | 0 | 0 |
| Vasilyevaea | 0 | 0 | 0.000159991 | 0 | 0 |
| Ancylobacter | 0 | 0 | 0.000106661 | 0 | 0.000130439 |
| Pedobacter | 0 | 0.001683598 | 0 | 0 | 0.000782636 |
| Acinetobacter | 0.625 | 0 | 0 | 0 | 0 |
| Bacillus | 0.375 | 0 | 0 | 0 | 0.0000521 |
| Ralstonia | 0 | 0 | 0 | 0 | 0.342168423 |
| Methylomicrobium | 0 | 0 | 0 | 0 | 0.023531253 |
| Stenotrophomonas | 0 | 0 | 0 | 0 | 0.009913388 |
| Pseudomonas | 0 | 0 | 0 | 0.000485771 | 0.009626422 |
| Rhodococcus | 0 | 0 | 0 | 0.000161924 | 0.008713347 |
| Delftia | 0 | 0 | 0 | 0 | 0.007017635 |
| Mycobacterium | 0 | 0 | 0 | 0 | 0.006417615 |
| Bradyrhizobium | 0 | 0 | 0 | 0 | 0.00610456 |
| Pseudonocardia | 0 | 0 | 0 | 0 | 0.004539288 |
| Vibrionimonas | 0 | 0 | 0 | 0 | 0.000991339 |
| Phreatobacter | 0 | 0 | 0 | 0 | 0.000939163 |
| Methylobacterium | 0 | 0 | 0 | 0 | 0.000547845 |
| Patulibacter | 0 | 0 | 0 | 0 | 0.000417406 |
| Dokdonella | 0 | 0 | 0 | 0 | 0.000391318 |
| Nocardioides | 0 | 0 | 0 | 0 | 0.00036523 |
| Bradyrhizobium-Oligotropha | 0 | 0 | 0 | 0 | 0.000313054 |
| Citrobacter | 0 | 0 | 0 | 0 | 0.000286967 |
| Williamsia | 0 | 0 | 0 | 0 | 0.000260879 |
| Nitrobacter | 0 | 0 | 0 | 0 | 0.000182615 |
| Phyllobacterium | 0 | 0 | 0 | 0 | 0.000182615 |
| Gordonia | 0 | 0 | 0 | 0 | 0.000130439 |
| Psychrobacter | 0 | 0 | 0 | 0 | 0.000104351 |
| Methylocystis-Methylosinus | 0 | 0 | 0 | 0 | 0.00007826 |
| Massilia | 0 | 0 | 0 | 0 | 0.00005217 |
| Algoriphagus | 0 | 0 | 0 | 0.001052504 | 0 |
| Rufibacter | 0 | 0 | 0 | 0.00008096 | 0 |

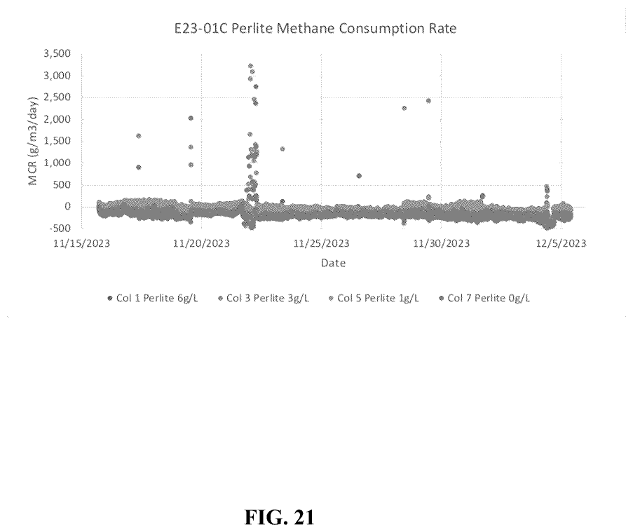

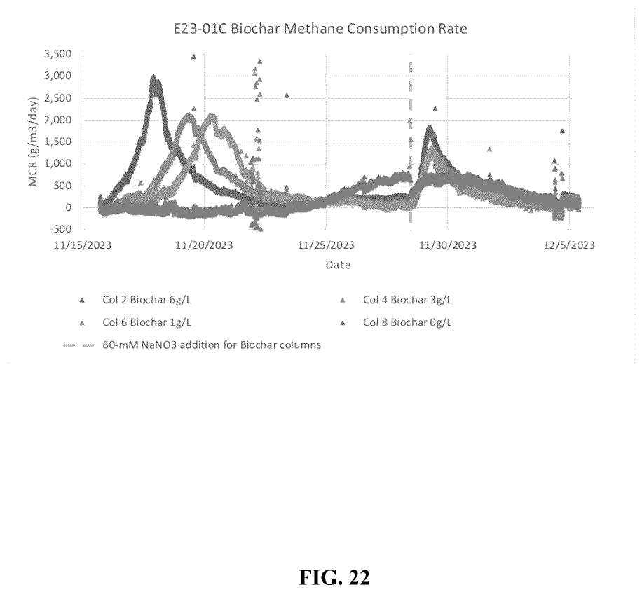

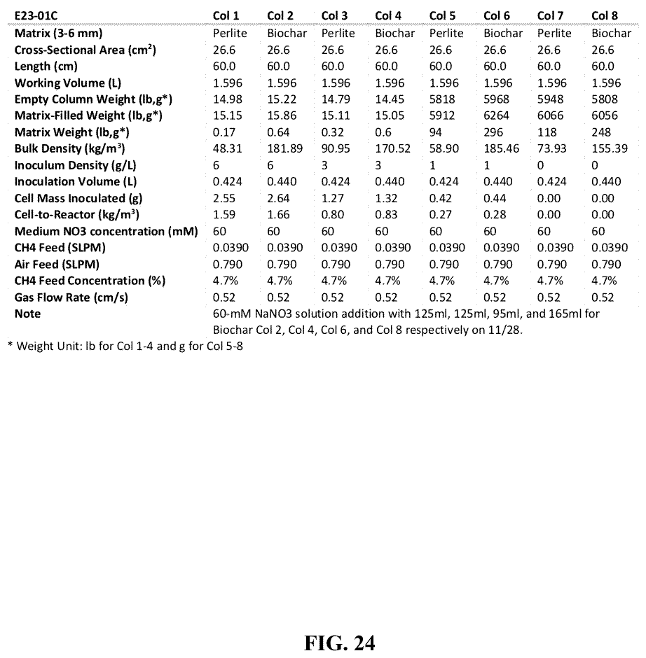

A second experiment was performed according to the details shown in FIG. 24. The inoculum concentrations in this experiment were lower (0 g/L, 1 g/L, 3 g/L, and 6 g/L) than those in the experiment described above. Similar to the experiment described above, the methane consumption rate was measured over time for each of the bacterial consortiums in each of the eight columns. The results of the perlite containing columns are shown in FIG. 21. In this case, the methane consumption rate was low for all samples. The results of the biochar containing columns are shown in FIG. 22. Similar to the experiment described above, the methane consumption rate increased initially for each of the columns followed by a decrease. After adding 60 mM NaNO₃, the rate again increased.

1-136. (canceled)

137. A composition comprising:
a) a consortium of bacteria comprising a methanotroph capable of fixing nitrogen (N₂) and consuming methane (CH₄); and
b) an additive, wherein the additive comprises tungsten, iron, nickel, molybdenum, cerium, manganese, lanthanum, cobalt, or boron.

138. The composition of claim 137, wherein the consortium of bacteria has a viability for at least 3 months.

139. The composition of claim 137, wherein the consortium of bacteria consumes methane (CH₄) at a rate greater than 5 micromoles (μmol)/gram of dry weight (gDW) per day.

140. The composition of claim 137, wherein the consortium of bacteria fixes nitrogen (N₂) at a rate greater than 2.0 μmol/gDW per day.

141. The composition of claim 137, wherein the consortium of bacteria has a neutral pH.

142. The composition 137, wherein the consortium of bacteria is freeze dried.

143. The composition of claim 137, wherein the one or more additives further comprises phosphorus, ethylenediaminetetraacetic acid, or a combination thereof.

144. The composition of claim 137, wherein the additive is in a sufficient amount to keep a bacterium of the consortium of bacteria consuming CH₄ at a population frequency of at least 0.01% for at least about 4 weeks.

145. The composition of claim 137, wherein the additive is present in the composition at a concentration of at least about 1 microgram (μg)/liter (L).

146. The composition of claim 137, wherein the additive increases CH₄ consumption by the consortium of bacteria compared to a reference composition comprising the consortium of bacteria and not having the additive.

147. The composition of claim 137, wherein the additive increases the viability of the consortium of bacteria compared to a reference composition comprising the consortium of bacteria and not having the additive.

148. A method of reducing emission of methane comprising applying the consortium of bacteria of claim 137 to a methane emission site, thereby reducing a concentration of methane emitted from the methane emission site.

149. The method of claim 148, wherein the methane emission site is a landfill.

150. The method of claim 148, wherein the methane emission site is a crop field.

151. The method of claim 148, wherein the methane emission field is an industrial plant.

152. The method of claim 148, wherein the applying comprises applying the consortium of bacteria as a liquid.

153. The method of claim 148, wherein the applying comprises applying the consortium of bacteria as a solid.

154. The method of claim 148, wherein the applying comprises applying the consortium of bacteria in a matrix.

155. The method of claim 154, wherein the matrix comprises biochar.

156. The method of claim 154, wherein the matrix comprises Perlite.

157. The method of claim 148, wherein the concentration of methane emitted from the methane emission site is reduced by at least about 20%.
